(19) United States
(12) Patent Application Publication
(10) Pub. No.: US 2026/0109746 A1
(43) Pub. Date: Apr. 23, 2026

(54) CD4-TARGETED IL-15 MOLECULES AND METHODS OF USE

(71) Applicant: Gilead Sciences, Inc., Foster City, CA (US)

(72) Inventors: Manuel Baca, Lexington, MA (US); Magdeleine S. Hung, Mountain View, CA (US); Alivelu M. Irrinki, San Jose, CA (US); Bo Jiang, Vista, CA (US); Jasmine Kaur, Fremont, CA (US); Daniel Keri, San Mateo, CA (US); Chingwei V. Lee, Foster City, CA (US); Prasenjit K. Mukherjee, South San Francisco, CA (US); Jeffrey P. Murry, Castro Valley, CA (US); Baljinder S. Randhawa, Castro Valley, CA (US); Majlinda K. Thomas, Menlo Park, CA (US); Hector Viadiu-Ilarraza, Palo Alto, CA (US)

(21) Appl. No.: 19/364,662

(22) Filed: Oct. 21, 2025

Related U.S. Application Data
(60) Provisional application No. 63/710,400, filed on Oct. 22, 2024.
(60) Provisional application No. 63/802,281, filed on May 8, 2025.

(51) Int. Cl. C07K 14/54; A61K 38/00; A61K 47/68; A61P 31/18; A61P 35/00; C07K 14/715; C07K 16/28

(52) U.S. Cl. CPC C07K 14/5443; A61K 47/6813; A61K 47/6849; A61P 31/18; A61P 35/00; C07K 14/7155; C07K 16/2812; A61K 38/00; C07K 2317/52; C07K 2317/565; C07K 2319/30

(57) ABSTRACT

Provided are CD4-targeted IL-15 variant molecules and methods for use.

### CROSS-REFERENCE TO RELATED APPLICATIONS

This application claims the benefit under 35 U.S.C. § 119(e) of U.S. Provisional Application No. 63/710,400, filed on Oct. 22, 2024, and U.S. Provisional Application No. 63/802,281, filed on May 8, 2025, which are hereby incorporated herein by reference in their entireties for all purposes.

### SEQUENCE LISTING

The instant application contains a Sequence Listing which has been submitted electronically in .XML format and is hereby incorporated by reference in its entirety. Said XML copy, created on Oct. 18, 2024, is named 1553-US-PSP_SL.xml and is 1,312,829 bytes in size.

### BACKGROUND

Human immunodeficiency virus (HIV) is the causative agent of the life-threatening illness, autoimmune deficiency syndrome (AIDS). Antiretroviral therapy (ART) can suppress viral replication and extend life, but it is unable to eliminate the HIV reservoir that persists within latently infected cells. This reservoir can exist in a quiescent state that makes it refractory to clearance by natural immunity. Latent HIV can persist for decades despite treatment, rapidly reseeding infection if treatment is interrupted. Lifelong therapy is associated with multiple comorbidities such as cardiovascular disease, diabetes and cancer. It is also associated with challenges related to tolerability, drug-drug interactions, resistance and adherence.

Many drugs that have been developed to treat other diseases have been tested as strategies for activating HIV expression. While many of these are effective in vitro, the doses that can be achieved in vivo are significantly limited by poor tolerability of these molecules (Siliciano et al., J Infect Dis (2021) 223(12 Suppl 2):13-21). Several common gamma chain (IL-2Rγ; IL2RG; CD132; NCBI Gene ID: 3561) cytokines, including IL-2 and IL-15, have been shown to activate HIV expression, but dosing of these has also been limited by poor tolerability (Chun et al., Nat Med (1999) 5:651-655; Miller et al., Nat Med (2022) 28:392-400). Mouse models indicate that the production of IFN-γ by NK cells mediates the immunotoxicity induced by IL-15-based therapeutics (Guo et al., J Immunol (2015) 195:2353-2364). This tolerability could potentially be improved by targeting IL-15 specifically to cells infected with HIV. However, early attempts at targeting cytokines to specific cellular markers showed limited effectiveness because the cytokines themselves have high affinity for their receptors (Tzeng et al., Proc Natl Acad Sci (2015) 112:3320-3325). The potential of IL-15 as a strategy for activating HIV expression is limited by the preferential activity seen with native IL-15 on CD4+ T cells. Although the HIV reservoir exists in both naïve and memory CD4+ T cells, IL-15 primarily activates memory CD4+ T cells (Kanegane et al., Blood (1996) 88:230-235).

### SUMMARY

Provided is an interleukin-15 variant (IL-15v) comprising the amino acid sequence of any one of SEQ ID NOs: 743, 750-752, 754-756, 758, 768-771, 774, 777, 779, 783-788, 790-798, 801-808, 810-813 and 1125. In some embodiments, the IL-15v binds to IL-2Rβ with a binding equilibrium dissociation constant (KD) of at least 1 μM, e.g., a KD of at least 1.5 μM, at least 2 μM, at least 2.5 μM, at least 3 μM, at least 3.5 μM, at least 4 μM, at least 4.5 μM or at least 5 μM. In some embodiments, the IL-15v binds to IL-2Rβγ with a binding equilibrium dissociation constant (KD) of at least 1 nM, e.g., a KD of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM.

Also provided is a fusion protein comprising: (i) an IL-15 receptor alpha subunit (IL15RA) SUSHI domain and (ii) an IL-15 variant (IL-15v), the fusion protein comprising the amino acid sequence of any one of SEQ ID NOs: 817, 824-826, 828-830, 832, 842-845, 848, 851, 853, 857-862, 864-872, 875-882, 884-887 and 1127. In some embodiments, the fusion protein comprises the amino acid sequence of any one of SEQ ID NOs: 1136, 1143-1145, 1147-1149, 1151, 1162-1165, 1168, 1171, 1173-1174, 1178-1183, 1185-1193 and 1196-1203 and 1205-1208. In some embodiments, the IL-15v binds to IL-2Rβ with a binding equilibrium dissociation constant (KD) of at least 1 μM, e.g., a KD of at least 1.5 μM, at least 2 μM, at least 2.5 μM, at least 3 μM, at least 3.5 μM, at least 4 μM, at least 4.5 μM or at least 5 μM. In some embodiments, the IL-15v binds to IL-2Rβγ with a binding equilibrium dissociation constant (KD) of at least 1 nM, e.g., a KD of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM. In some embodiments, the IL-15v induces CD4+ T cell proliferation with an EC50 of less than 5 nM, e.g., less than 4 nM, less than 3 nM, less than 2 nM, less than 1 nM, e.g., wherein CD4+ T cell proliferation potency is measured by marker of proliferation Ki-67 activation (MKI67; NCBI Gene ID: 4288). In some embodiments, the IL-15v induces CD8+ T cells and/or natural killer (NK) cell proliferation with an EC50 of greater than 100 nM, e.g., wherein CD8+ T cell and/or NK cell proliferation potency is measured by Ki-67 activation.

With respect to further embodiments of the IL-15v or the IL-15RA SUSHI-IL-15v fusion protein, some embodiments, the IL-15v is PEGylated. In some embodiments, the IL-15v or the fusion protein is fused to an immunoglobulin crystallizable fragment (Fc). In some embodiments, the IL-15v or the fusion protein comprises a transmembrane domain.

Further provided is a chimeric antigen receptor (CAR) comprising the IL-15v or the IL-15RA SUSHI-IL-15v fusion protein, as described above and herein. Also provided is a CAR-T cell transfected with a polynucleotide that encodes the IL-15v or the IL-15RA SUSHI-IL-15v fusion protein, as described above and herein. Further provided is a host cell transfected with a polynucleotide that encodes the IL-15v or the IL-15RA SUSHI-IL-15v fusion protein, as described above and herein.

Further provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of: SEQ ID NOs: 1, 2, 3, 4, 5 and 6 (1); SEQ ID NOs: 1, 7, 3, 4, 8 and 9 (2); SEQ ID NOs: 1, 10, 3, 11, 5 and 6 (3); SEQ ID NOs: 1, 10, 3, 11, 12 and 6 (4); SEQ ID NOs: 1, 10, 3, 11, 8 and 9 (5); SEQ ID NOs: 1, 13, 3, 11, 8 and 9 (6); SEQ ID NOs: 1, 14, 3, 11, 8 and 9 (7); SEQ ID NOs: 1, 15, 3, 11, 8 and 9 (8); SEQ ID NOs: 1, 16, 3, 11, 8 and 9 (9); SEQ ID NOs: 1, 15, 3, 11, 12 and 17 (10); SEQ ID NOs: 1, 16, 3, 11, 12 and 18 (11); SEQ ID NOs: 1, 1078, 3, 11, 8 and 9 (12); SEQ ID NOs: 1, 1079, 3, 11, 8 and 9 (13, 14, 17, 18 and 19); SEQ ID NOs: 1, 1080, 3, 11, 8 and 9 (15); SEQ ID NOs: 1, 1081, 3, 11, 8 and 9 (16); SEQ ID NOs: 19, 20, 21, 22, 23 and 24 (1.17); SEQ ID NOs: 25, 26, 27, 28, 29 and 30 (1.25); SEQ ID NOs: 31, 32, 33, 34, 29 and 35 (1.26); SEQ ID NOs: 25, 36, 37, 28, 29 and 38 (1.27); SEQ ID NOs: 25, 1082, 39, 28, 29 and 38 (1.28); SEQ ID NOs: 40, 41, 42, 28, 29 and 35 (1.29); SEQ ID NOs: 25, 43, 39, 28, 29 and 38 (1.30); SEQ ID NOs: 25, 44, 45, 28, 29 and 35 (1.31); SEQ ID NOs: 25, 46, 47, 28, 29 and 35 (1.32); SEQ ID NOs: 25, 48, 49, 28, 29 and 35 (1.33); SEQ ID NOs: 25, 48, 50, 28, 29 and 35 (1.34); SEQ ID NOs: 51, 52, 53, 54, 55 and 56 (1.45) or SEQ ID NOs: 51, 57, 53, 58, 55 and 56 (1.46, 1.47, 1.48). In some embodiments, the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 625 and 626 (1); SEQ ID NOs: 627 and 628 (2); SEQ ID NOs: 629 and 630 (3); SEQ ID NOs: 629 and 631 (4); SEQ ID NOs: 629 and 632 (5); SEQ ID NOs: 633 and 632 (6); SEQ ID NOs: 634 and 632 (7); SEQ ID NOs: 635 and 632 (8); SEQ ID NOs: 636 and 632 (9); SEQ ID NOs: 635 and 637 (10); SEQ ID NOs: 636 and 638 (11); SEQ ID NOs: 639 and 632 (12); SEQ ID NOs: 640 and 632 (13); SEQ ID NOs: 641 and 632 (14); SEQ ID NOs: 642 and 632 (15); SEQ ID NOs: 643 and 632 (16); SEQ ID NOS: 640 and 644 (17); SEQ ID NOs: 640 and 645 (18); SEQ ID NOs: 640 and 646 (19); SEQ ID NOs: 647 and 648 (1.17); SEQ ID NOs: 649 and 650 (1.25); SEQ ID NOs: 651 and 652 (1.26); SEQ ID NOs: 653 and 654 (1.27); SEQ ID NOs: 655 and 656 (1.28); SEQ ID NOs: 657 and 658 (1.29); SEQ ID NOs: 659 and 660 (1.30); SEQ ID NOs: 661 and 662 (1.31); SEQ ID NOs: 663 and 664 (1.32); SEQ ID NOs: 665 and 666 (1.33); SEQ ID NOs: 667 and 666 (1.34); SEQ ID NOs: 668 and 669 (1.45); SEQ ID NOs: 670 and 671 (1.46); SEQ ID NOs: 672 and 673 (1.47) or SEQ ID NOs: 672 and 671 (1.48).

Further provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of: SEQ ID NOs: 25, 83, 84, 85, 86 and 87 (1.1); SEQ ID NOs: 88, 89, 90, 91, 92 and 93 (1.4); SEQ ID NOs: 94, 95, 96, 97, 98 and 99 (1.5); SEQ ID NOs: 88, 100, 101, 102, 103 and 104 (2.9); SEQ ID NOs: 88, 105, 106, 107, 108 and 93 (2.10); SEQ ID NOs: 88, 109, 110, 111, 108 and 112 (2.11); SEQ ID NOs: 88, 109, 110, 111, 113 and 112 (2.11 VL N50G); SEQ ID NOs: 88, 114, 106, 107, 108 and 115 (2.12); SEQ ID NOs: 88, 114, 106, 107, 113 and 115 (2.12 VL N50G); SEQ ID NOs: 88, 116, 117, 118, 108 and 119 (2.13); SEQ ID NOs: 120, 121, 122, 102, 113 and 123 (2.14) or SEQ ID NOs: 88, 124, 122, 125, 113 and 104 (2.15). In some embodiments, the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 682 and 683 (1.1); SEQ ID NOs: 684 and 685 (1.4); SEQ ID NOs: 686 and 687 (1.5); SEQ ID NOs: 688 and 689 (2.9); SEQ ID NOs: 690 and 691 (2.10); SEQ ID NOs: 692 and 693 (2.11); SEQ ID NOs: 692 and 694 (2.11 VL N50G); SEQ ID NOs: 695 and 696 (2.12); SEQ ID NOs: 695 and 697 (2.12 VL N50G); SEQ ID NOs: 698 and 699 (2.13); SEQ ID NOs: 700 and 701 (2.14) or SEQ ID NOs: 702 and 703 (2.15). In some embodiments, the antibody or antigen-binding fragment thereof is insensitive to (i.e., binds to CD4 in the presence and absence of) the amino acid substitution resulting from CD4 polymorphism variant ID rs11064416 (F123L). Further provided is a means for binding to CD4 D2 that is insensitive to amino acid substitution resulting from polymorphism variant ID rs11064416 (F123L).

Further provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of: SEQ ID NOs: 126, 127, 128, 129, 130 and 99 (1.6); SEQ ID NOs: 126, 131, 132, 129, 133 and 99 (1.7); SEQ ID NOs: 126, 134, 135, 136, 133 and 99 (1.8); SEQ ID NOs: 126, 137, 128, 129, 138 and 99 (1.9); SEQ ID NOs: 126, 139, 128, 140, 141 and 99 (1.10); SEQ ID NOs: 142, 143, 128, 145, 141 and 99 (1.19); SEQ ID NOs: 126, 146, 147, 140, 141 and 99 (2.2) or SEQ ID NOs: 148, 149, 150, 129, 151 and 99 (2.3). In some embodiments, the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 704 and 705 (1.6); SEQ ID NOs: 706 and 707 (1.7); SEQ ID NOs: 708 and 709 (1.8); SEQ ID NOs: 710 and 711 (1.9); SEQ ID NOs: 712 and 713 (1.10); SEQ ID NOs: 714 and 715 (1.19); SEQ ID NOs: 716 and 717 (2.2) or SEQ ID NOs: 718 and 719 (2.3). In some embodiments, the antibody or antigen-binding fragment thereof is insensitive to (i.e., binds to CD4 in the presence or absence of) the amino acid substitutions resulting from one or more of CD4 polymorphism variant IDs rs28919570 (R265W), rs11064419 (F227S or F227C) and rs11064416 (F123L). Further provided is a means for binding to one or both of CD4 D2 and D3 that is insensitive to amino acid substitution resulting from polymorphism variant IDs rs28919570 (R265W), rs11064419 (F227S or F227C) and rs11064416 (F123L).

Further provided is antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of: SEQ ID NOs: 158, 159, 160, 145, 161 and 162 (1.20); SEQ ID NOs: 163, 164, 160, 165, 166 and 162 (1.21); SEQ ID NOs: 163, 164, 160, 165, 161 and 162 (1.22); SEQ ID NOs: 158, 167, 168, 145, 98 and 169 (1.23); SEQ ID NOs: 170, 171, 172, 173, 141 and 99 (2.1) or SEQ ID NOs: 25, 174, 175, 176, 29 and 177 (2.8). In some embodiments, the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 722 and 723 (1.20); SEQ ID NOs: 724 and 725 (1.21); SEQ ID NOs: 724 and 726 (1.22); SEQ ID NOs: 727 and 728 (1.23); SEQ ID NOs: 727 and 729 (1.23 VL C36Y); SEQ ID NOs: 730 and 731 (2.1) or SEQ ID NOs: 732 and 733 (2.8). In some embodiments, the antibody or antigen-binding fragment thereof is insensitive to (i.e., binds to CD4 in the presence or absence of) the amino acid substitutions resulting from one or more of CD4 polymorphism variant IDs rs28919570 (R265W) and rs11064419 (F227S or F227C). In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120. Further provided is a means for binding to CD4 D3 that is insensitive to amino acid substitution resulting from polymorphism variant IDs rs28919570 (R265W) and rs11064419 (F227S or F227C). In some embodiments, the means binds to CD4 with an equilibrium dissociation constant (KD) of lower than 1 nM, e.g., lower than 100 pM, lower than 90 pM, lower than 80 pM, lower than 70 pM, lower than 60 pM, lower than 50 pM, lower than 40 pM, or lower than 30 pM.

With respect to further embodiments of the CD4-binding antibody or antigen-binding fragment thereof (D1, D2, D2/D3 or D3), in some embodiments, the antibody or antigen-binding fragment thereof binds to CD4 with an equilibrium dissociation constant (K_{D}) of lower than 1 nM, e.g., lower than 100 pM, lower than 90 pM, lower than 80 pM, lower than 70 pM, lower than 60 pM, lower than 50 pM, lower than 40 pM, or lower than 30 pM.

Further provided is a CD4-targeted interleukin-15 (IL-15) molecule comprising: (a) a first polypeptide comprising an immunoglobulin heavy chain comprising a first immunoglobulin fragment crystallizable domain (Fc domain); (b) a second polypeptide comprising an immunoglobulin light chain (VL-CL), wherein the first polypeptide and the second polypeptide form an antigen binding domain that specifically binds to CD4; and (c) a third polypeptide comprising a fusion protein comprising: (i) an IL-15 receptor alpha subunit (IL15RA) SUSHI domain, (ii) an IL-15 or variant thereof (IL-15v), and (iii) a second Fc domain, wherein the first Fc domain and the second Fc domain heterodimerize to form a bispecific molecule that binds to CD4 and an IL-2βγ complex (CD122 and CD132). In some embodiments, the antigen binding domain that specifically binds to CD4 competes with or comprises a heavy chain variable region (VH) and a light chain variable region (VL) from an antibody selected from the group consisting of ibalizumab, tregalizumab, keliximab, zanolimumab, clenoliximab, priliximab, UB-421, RPA-T4, SK3, MEM241 and OKT-4.

In some embodiments of the CD4-targeted IL-15 molecule, the antigen binding domain that specifically binds to CD4 specifically binds to the CD4 D1 domain. In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of: SEQ ID NOs: 1, 2, 3, 4, 5 and 6 (1); SEQ ID NOs: 1, 7, 3, 4, 8 and 9 (2); SEQ ID NOs: 1, 10, 3, 11, 5 and 6 (3); SEQ ID NOs: 1, 10, 3, 11, 12 and 6 (4); SEQ ID NOs: 1, 10, 3, 11, 8 and 9 (5); SEQ ID NOs: 1, 13, 3, 11, 8 and 9 (6); SEQ ID NOs: 1, 14, 3, 11, 8 and 9 (7); SEQ ID NOs: 1, 15, 3, 11, 8 and 9 (8); SEQ ID NOs: 1, 16, 3, 11, 8 and 9 (9); SEQ ID NOs: 1, 15, 3, 11, 12 and 17 (10); SEQ ID NOs: 1, 16, 3, 11, 12 and 18 (11); SEQ ID NOs: 1, 1078, 3, 11, 8 and 9 (12); SEQ ID NOs: 1, 1079, 3, 11, 8 and 9 (13, 14, 17, 18 and 19); SEQ ID NOs: 1, 1080, 3, 11, 8 and 9 (15); SEQ ID NOs: 1, 1081, 3, 11, 8 and 9 (16); SEQ ID NOs: 19, 20, 21, 22, 23 and 24 (1.17); SEQ ID NOs: 25, 26, 27, 28, 29 and 30 (1.25); SEQ ID NOs: 31, 32, 33, 34, 29 and 35 (1.26); SEQ ID NOs: 25, 36, 37, 28, 29 and 38 (1.27); SEQ ID NOs: 25, 1082, 39, 28, 29 and 38 (1.28); SEQ ID NOs: 40, 41, 42, 28, 29 and 35 (1.29); SEQ ID NOs: 25, 43, 39, 28, 29 and 38 (1.30); SEQ ID NOs: 25, 44, 45, 28, 29 and 35 (1.31); SEQ ID NOs: 25, 46, 47, 28, 29 and 35 (1.32); SEQ ID NOs: 25, 48, 49, 28, 29 and 35 (1.33); SEQ ID NOs: 25, 48, 50, 28, 29 and 35 (1.34); SEQ ID NOs: 51, 52, 53, 54, 55 and 56 (1.45); SEQ ID NOs: 51, 57, 53, 58, 55 and 56 (1.46, 1.47, 1.48); SEQ ID NOs: 59, 60, 61, 62, 63 and 64 (keliximab) or SEQ ID NOs: 65, 66, 67, 68, 69 and 70 (UB-421). In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of: SEQ ID NOs: 178, 179, 180, 181, 182 and 6 (1); SEQ ID NOs: 178, 183, 180, 181, 182 and 9 (2); SEQ ID NOs: 178, 184, 180, 181, 182 and 6 (4); SEQ ID NOs: 178, 184, 180, 181, 182 and 9 (3, 5); SEQ ID NOs: 178, 185, 180, 181, 182 and 9 (6, 7, 12); SEQ ID NOs: 178, 186, 180, 181, 182 and 9 (8); SEQ ID NOs: 178, 187, 180, 181, 182 and 9 (9); SEQ ID NOs: 178, 186, 180, 181, 182 and 17 (10); SEQ ID NOs: 178, 187, 180, 181, 182 and 18 (11); SEQ ID NOs: 178, 188, 180, 181, 182 and 9 (13, 14, 17, 18, 19); SEQ ID NOs: 178, 189, 180, 181, 182 and 9 (15); SEQ ID NOs: 178, 190, 180, 181, 182 and 9 (16); SEQ ID NOs: 191, 192, 193, 194, 195 and 24 (1.17); SEQ ID NOs: 196, 197, 198, 199, 200 and 30 (1.25); SEQ ID NOs: 201, 202, 203, 199, 200 and 35 (1.26); SEQ ID NOs: 204, 205, 206, 199, 200 and 38 (1.27); SEQ ID NOs: 196, 207, 208, 199, 200 and 38 (1.28); SEQ ID NOs: 209, 210, 211, 199, 200 and 35 (1.29); SEQ ID NOs: 196, 212, 208, 199, 200 and 38 (1.30); SEQ ID NOs: 196, 213, 214, 199, 200 and 35 (1.31); SEQ ID NOs: 196, 213, 215, 199, 200 and 35 (1.32); SEQ ID NOs: 196, 216, 217, 199, 200 and 35 (1.33); SEQ ID NOs: 196, 216, 218, 199, 200 and 35 (1.34); SEQ ID NOs: 219, 220, 221, 222, 223 and 56 (1.45); SEQ ID NOs: 219, 224, 221, 225, 223 and 56 (1.46, 1.47, 1.48); SEQ ID NOs: 226, 227, 228, 229, 230 and 64 (keliximab) or SEQ ID NOs: 231, 232, 233, 234, 235 and 70 (UB-421). In some embodiments, the antigen binding domain that specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of: SEQ ID NOs: 323, 324, 325, 326, 182 and 327 (1); SEQ ID NOs: 323, 328, 325, 326, 182 and 329 (2); SEQ ID NOs: 323, 330, 325, 326, 182 and 327 (4); SEQ ID NOs: 323, 330, 325, 326, 182 and 329 (5); SEQ ID NOs: 323, 331, 325, 326, 182 and 329 (6, 7, 12); SEQ ID NOs: 323, 332, 325, 326, 182 and 329 (8); SEQ ID NOs: 323, 333, 325, 326, 182 and 329 (9); SEQ ID NOs: 323, 332, 325, 326, 182 and 334 (10); SEQ ID NOs: 323, 333, 325, 326, 182 and 335 (11); SEQ ID NOs: 323, 336, 325, 326, 182 and 329 (13, 14, 17, 18 and 19); SEQ ID NOs: 323, 337, 325, 326, 182 and 329 (15); SEQ ID NOs: 323, 338, 325, 326, 182 and 329 (16); SEQ ID NOs: 339, 340, 341, 342, 195 and 1131 (1.17); SEQ ID NOs: 344, 345, 346, 347, 200 and 348 (1.25); SEQ ID NOs: 349, 350, 351, 347, 200 and 352 (1.26); SEQ ID NOs: 353, 354, 355, 347, 200 and 356 (1.27); SEQ ID NOs: 344, 350, 357, 347, 200 and 356 (1.28); SEQ ID NOs: 358, 350, 359, 347, 200 and 352 (1.29); SEQ ID NOs: 343, 354, 357, 347, 200 and 356 (1.30); SEQ ID NOs: 343, 360, 361, 347, 200 and 352 (1.31); SEQ ID NOs: 343, 360, 362, 347, 200 and 352 (1.32); SEQ ID NOs: 343, 363, 364, 347, 200 and 352 (1.33); SEQ ID NOs: 343, 363, 365, 347, 200 and 352 (1.34); SEQ ID NOs: 366, 367, 368, 1084, 223 and 369 (1.45); SEQ ID NOs: 366, 367, 368, 370, 223 and 369 (1.46, 1.47, 1.48); SEQ ID NOs: 371, 372, 373, 374, 230 and 375 (keliximab) or SEQ ID NOs: 376, 377, 378, 379, 235 and 380 (UB-421). In some embodiments, the antigen binding domain that specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of: SEQ ID NOs: 463, 464, 465, 466, 467 and 327 (1); SEQ ID NOs: 463, 468, 465, 466, 469 and 329 (2); SEQ ID NOs: 463, 470, 465, 466, 467 and 327 (3); SEQ ID NOs: 463, 470, 465, 466, 469 and 327 (4); SEQ ID NOs: 463, 470, 465, 466, 469 and 329 (5); SEQ ID NOs: 463, 1132, 465, 466, 469 and 329 (6); SEQ ID NOs: 463, 471, 465, 466, 469 and 329 (7); SEQ ID NOs: 463, 472, 465, 466, 469 and 329 (8); SEQ ID NOs: 463, 473, 465, 466, 469 and 329 (9); SEQ ID NOs: 463, 472, 465, 466, 474 and 334 (10); SEQ ID NOs: 463, 473, 465, 466, 474 and 335 (11); SEQ ID NOs: 463, 475, 465, 466, 469 and 329 (12); SEQ ID NOs: 463, 478, 465, 466, 469 and 329 (13, 14, 17, 18 and 19); SEQ ID NOs: 463, 476, 465, 466, 469 and 329 (15); SEQ ID NOs: 463, 477, 465, 466, 469 and 329 (16); SEQ ID NOs: 479, 480, 481, 482, 483 and 1131 (1.17); SEQ ID NOs: 484, 485, 486, 487, 488 and 348 (1.25); SEQ ID NOs: 489, 490, 491, 487, 492 and 352 (1.26); SEQ ID NOs: 493, 494, 495, 487, 492 and 356 (1.27); SEQ ID NOs: 484, 496, 497, 487, 492 and 356 (1.28); SEQ ID NOs: 498, 499, 500, 487, 492 and 352 (1.29); SEQ ID NOs: 484, 501, 497, 487, 492 and 356 (1.30); SEQ ID NOs: 484, 502, 503, 487, 492 and 352 (1.31); SEQ ID NOs: 484, 504, 505, 487, 506 and 352 (1.32); SEQ ID NOs: 484, 507, 508, 487, 492 and 352 (1.33); SEQ ID NOs: 484, 507, 509, 487, 492 and 352 (1.34); SEQ ID NOs: 510, 511, 512, 513, 514 and 369 (1.45); SEQ ID NOs: 510, 515, 512, 516, 514 and 369 (1.46, 1.47, 1.48); SEQ ID NOs: 517, 518, 519, 374, 520 and 375 (keliximab) or SEQ ID NOs: 521, 522, 523, 524, 525 and 380 (UB-421). In some embodiments, the antigen binding domain that specifically binds to CD4 D1 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 625 and 626 (1); SEQ ID NOs: 627 and 628 (2); SEQ ID NOs: 629 and 630 (3); SEQ ID NOs: 629 and 631 (4); SEQ ID NOs: 629 and 632 (5); SEQ ID NOs: 633 and 632 (6); SEQ ID NOs: 634 and 632 (7); SEQ ID NOs: 635 and 632 (8); SEQ ID NOs: 636 and 632 (9); SEQ ID NOs: 635 and 637 (10); SEQ ID NOs: 636 and 638 (11); SEQ ID NOs: 639 and 632 (12); SEQ ID NOs: 640 and 632 (13); SEQ ID NOs: 641 and 632 (14); SEQ ID NOs: 642 and 632 (15); SEQ ID NOs: 643 and 632 (16); SEQ ID NOs: 640 and 644 (17); SEQ ID NOs: 640 and 645 (18); SEQ ID NOs: 640 and 646 (19); SEQ ID NOs: 647 and 648 (1.17); SEQ ID NOs: 649 and 650 (1.25); SEQ ID NOs: 651 and 652 (1.26); SEQ ID NOs: 653 and 654 (1.27); SEQ ID NOs: 655 and 656 (1.28); SEQ ID NOs: 657 and 658 (1.29); SEQ ID NOs: 659 and 660 (1.30); SEQ ID NOs: 661 and 662 (1.31); SEQ ID NOs: 663 and 664 (1.32); SEQ ID NOs: 665 and 666 (1.33); SEQ ID NOs: 667 and 666 (1.34); SEQ ID NOs: 668 and 669 (1.45); SEQ ID NOs: 670 and 671 (1.46); SEQ ID NOs: 672 and 673 (1.47); SEQ ID NOs: 672 and 671 (1.48); SEQ ID NOs: 674 and 675 (keliximab) or SEQ ID NOs: 676 and 677 (UB-421).

In some embodiments of the CD4-targeted IL-15 molecule, the antigen binding domain that specifically binds to CD4 specifically binds to the CD4 D2 domain. In some embodiments, the antigen binding domain specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of: SEQ ID NOs: 71, 72, 73, 74, 75 and 76 (ibalizumab); SEQ ID NOs: 77, 78, 79, 80, 81 and 82 (tregalizumab); SEQ ID NOs: 25, 83, 84, 85, 86 and 87 (1.1); SEQ ID NOs: 88, 89, 90, 91, 92 and 93 (1.4); SEQ ID NOs: 94, 95, 96, 97, 98 and 99 (1.5); SEQ ID NOs: 88, 100, 101, 102, 103 and 104 (2.9); SEQ ID NOs: 88, 105, 106, 107, 108 and 93 (2.10); SEQ ID NOs: 88, 109, 110, 111, 108 and 112 (2.11); SEQ ID NOs: 88, 109, 110, 111, 113 and 112 (2.11 VL N50G); SEQ ID NOs: 88, 114, 106, 107, 108 and 115 (2.12); SEQ ID NOs: 88, 114, 106, 107, 113 and 115 (2.12 VL N50G); SEQ ID NOs: 88, 116, 117, 118, 108 and 119 (2.13); SEQ ID NOs: 120, 121, 122, 102, 113 and 123 (2.14) or SEQ ID NOs: 88, 124, 122, 125, 113 and 104 (2.15). In some embodiments, the antigen binding domain specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of: SEQ ID NOs: 236, 237, 238, 239, 240 and 76 (ibalizumab); SEQ ID NOs: 241, 242, 243, 244, 245 and 82 (tregalizumab); SEQ ID NOs: 246, 247, 248, 249, 250 and 87 (1.1); SEQ ID NOs: 251, 252, 253, 254, 255 and 93 (1.4); SEQ ID NOs: 256, 257, 258, 259, 255 and 99 (1.5); SEQ ID NOs: 251, 260, 261, 262, 263 and 104 (2.9); SEQ ID NOs: 251, 264, 265, 266, 267 and 93 (2.10); SEQ ID NOs: 251, 268, 269, 270, 267 and 112 (2.11); SEQ ID NOs: 251, 268, 269, 270, 263 and 112 (2.11 VL N50G); SEQ ID NOs: 251, 271, 265, 266, 267 and 115 (2.12); SEQ ID NOs: 251, 271, 265, 266, 263 and 115 (2.12 VL N50G); SEQ ID NOS: 251, 272, 273, 274, 267 and 119 (2.13); SEQ ID NOs: 275, 276, 277, 262, 263 and 123 (2.14) or SEQ ID NOs: 251, 278, 277, 279, 263 and 104 (2.15). In some embodiments, the antigen binding domain that specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of: SEQ ID NOs: 366, 381, 382, 383, 240 and 384 (ibalizumab); SEQ ID NOs: 385, 386, 387, 388, 245 and 389 (tregalizumab); SEQ ID NOs: 390, 391, 392, 393, 250 and 394 (1.1); SEQ ID NOs: 395, 396, 397, 398, 255 and 399 (1.4); SEQ ID NOs: 400, 401, 402, 403, 255 and 404 (1.5); SEQ ID NOs: 395, 405, 406, 407, 263 and 408 (2.9); SEQ ID NOs: 395, 409, 410, 411, 267 and 399 (2.10); SEQ ID NOs: 395, 412, 413, 414, 267 and 399 (2.11); SEQ ID NOs: 395, 412, 413, 414, 263 and 399 (2.11 VL N50G); SEQ ID NOs: 395, 415, 410, 411, 267 and 408 (2.12); SEQ ID NOs: 395, 415, 410, 411, 263 and 408 (2.12 VL N50G); SEQ ID NOs: 395, 416, 417, 418, 267 and 419 (2.13); SEQ ID NOs: 420, 421, 422, 407, 263 and 408 (2.14) or SEQ ID NOs: 395, 423, 422, 424, 263 and 408 (2.15). In some embodiments, the antigen binding domain that specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of: SEQ ID NOs: 526, 527, 528, 529, 530 and 384 (ibalizumab); SEQ ID NOs: 531, 532, 533, 534, 535 and 389 (tregalizumab); SEQ ID NOs: 536, 537, 538, 539, 540 and 394 (1.1); SEQ ID NOs: 541, 542, 543, 544, 545 and 399 (1.4); SEQ ID NOs: 546, 547, 548, 549, 550 and 404 (1.5); SEQ ID NOs: 541, 551, 552, 553, 554 and 408 (2.9); SEQ ID NOs: 541, 555, 556, 557, 558 and 399 (2.10); SEQ ID NOs: 541, 559, 560, 561, 562 and 399 (2.11); SEQ ID NOs: 541, 559, 560, 561, 563 and 399 (2.11 VL N50G); SEQ ID NOs: 541, 564, 556, 557, 558 and 408 (2.12); SEQ ID NOs: 541, 564, 556, 557, 565 and 408 (2.12 VL N50G); SEQ ID NOs: 541, 566, 567, 568, 569 and 419 (2.13); SEQ ID NOs: 570, 571, 572, 553, 565 and 408 (2.14) or SEQ ID NOs: 541, 573, 572, 574, 575 and 408 (2.15). In some embodiments, the antigen binding domain that specifically binds to CD4 D2 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 678 and 679 (ibalizumab); SEQ ID NOs: 680 and 681 (tregalizumab); SEQ ID NOs: 682 and 683 (1.1); SEQ ID NOs: 684 and 685 (1.4); SEQ ID NOs: 686 and 687 (1.5); SEQ ID NOs: 688 and 689 (2.9); SEQ ID NOs: 690 and 691 (2.10); SEQ ID NOs: 692 and 693 (2.11); SEQ ID NOs: 692 and 694 (2.11 VL N50G); SEQ ID NOs: 695 and 696 (2.12); SEQ ID NOs: 695 and 697 (2.12 VL N50G); SEQ ID NOs: 698 and 699 (2.13); SEQ ID NOs: 700 and 701 (2.14) or SEQ ID NOs: 702 and 703 (2.15). In some embodiments, the antibody or antigen-binding fragment thereof is insensitive to (i.e., binds to CD4 in the presence and absence of) the amino acid substitution resulting from CD4 polymorphism variant ID rs11064416 (F123L).

In some embodiments of the CD4-targeted IL-15 molecule, the antigen binding domain that specifically binds to CD4 specifically binds to one or both of the CD4 D2 domain and the CD4 D3 domain. In some embodiments, the antigen binding domain specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of: SEQ ID NOs: 126, 127, 128, 129, 130 and 99 (1.6); SEQ ID NOs: 126, 131, 132, 129, 133 and 99 (1.7); SEQ ID NOs: 126, 134, 135, 136, 133 and 99 (1.8); SEQ ID NOs: 126, 137, 128, 129, 138 and 99 (1.9); SEQ ID NOs: 126, 139, 128, 140, 141 and 99 (1.10); SEQ ID NOs: 142, 143, 128, 145, 141 and 99 (1.19); SEQ ID NOs: 126, 146, 147, 140, 141 and 99 (2.2) or SEQ ID NOs: 148, 149, 150, 129, 151 and 99 (2.3). In some embodiments, the antigen binding domain specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of: SEQ ID NOs: 280, 281, 282, 283, 284 and 99 (1.6); SEQ ID NOs: 280, 285, 286, 283, 284 and 99 (1.7); SEQ ID NOs: 280, 287, 288, 283, 284 and 99 (1.8); SEQ ID NOs: 289, 290, 282, 283, 284 and 99 (1.9); SEQ ID NOs: 289, 291, 292, 293, 255 and 99 (1.10); SEQ ID NOs: 294, 295, 296, 297, 255 and 99 (1.19); SEQ ID NOs: 289, 298, 299, 293, 255 and 99 (2.2) or SEQ ID NOs: 289, 300, 301, 283, 284 and 99 (2.3). In some embodiments, the antigen binding domain that specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of: SEQ ID NOs: 425, 426, 427, 428, 284 and 404 (1.6); SEQ ID NOs: 425, 426, 429, 428, 284 and 404 (1.7); SEQ ID NOs: 425, 426, 430, 428, 284 and 404 (1.8); SEQ ID NOs: 431, 432, 427, 428, 284 and 404 (1.9); SEQ ID NOs: 431, 433, 427, 434, 255 and 404 (1.10); SEQ ID NOs: 431, 435, 436, 437, 255 and 404 (1.19); SEQ ID NOs: 431, 438, 439, 434, 255 and 404 (2.2) or SEQ ID NOs: 431, 438, 440, 428, 284 and 404 (2.3). In some embodiments, the antigen binding domain that specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of: SEQ ID NOs: 576, 577, 578, 579, 580 and 404 (1.6); SEQ ID NOs: 576, 581, 582, 579, 583 and 404 (1.7); SEQ ID NOs: 576, 584, 585, 586, 583 and 404 (1.8); SEQ ID NOs: 587, 588, 578, 579, 589 and 404 (1.9); SEQ ID NOs: 587, 590, 578, 591, 592 and 404 (1.10); SEQ ID NOs: 593, 594, 595, 596, 597 and 404 (1.19); SEQ ID NOs: 587, 598, 599, 591, 597 and 404 (2.2) or SEQ ID NOs: 587, 600, 601, 579, 602 and 404 (2.3). In some embodiments, the antigen binding domain that specifically binds to one or both of CD4 D2 and CD4 D3 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 704 and 705 (1.6); SEQ ID NOs: 706 and 707 (1.7); SEQ ID NOs: 708 and 709 (1.8); SEQ ID NOs: 710 and 711 (1.9); SEQ ID NOs: 712 and 713 (1.10); SEQ ID NOs: 714 and 715 (1.19); SEQ ID NOs: 716 and 717 (2.2) or SEQ ID NOs: 718 and 719 (2.3). In some embodiments, the antibody or antigen-binding fragment thereof is insensitive to (i.e., binds to CD4 in the presence or absence of) the amino acid substitutions resulting from one or more of CD4 polymorphism variant IDs rs28919570 (R265W), rs11064419 (F227S or F227C) and rs11064416 (F123L).

In some embodiments of the CD4-targeted IL-15 molecule, the antigen binding domain that specifically binds to CD4 specifically binds to the CD4 D3 domain. In some embodiments, the antigen binding domain specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of: SEQ ID NOs: 152, 153, 154, 155, 156 and 157 (OKT4); SEQ ID NOs: 158, 159, 160, 145, 161 and 162 (1.20); SEQ ID NOs: 163, 164, 160, 165, 166 and 162 (1.21); SEQ ID NOs: 163, 164, 160, 165, 161 and 162 (1.22); SEQ ID NOs: 158, 167, 168, 145, 98 and 169 (1.23); SEQ ID NOs: 170, 171, 172, 173, 141 and 99 (2.1) or SEQ ID NOs: 25, 174, 175, 176, 29 and 177 (2.8). In some embodiments, the antigen binding domain specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of: SEQ ID NOs: 302, 303, 304, 305, 245 and 157 (OKT4); SEQ ID NOs: 306, 307, 1083, 297, 308 and 162 (1.20); SEQ ID NOs: 309, 310, 311, 312, 313 and 162 (1.21); SEQ ID NOs: 309, 310, 311, 312, 308 and 162 (1.22); SEQ ID NOs: 314, 315, 316, 297, 255 and 169 (1.23); SEQ ID NOs: 317, 318, 319, 320, 255 and 99 (2.1) or SEQ ID NOs: 196, 321, 322, 199, 200 and 177 (2.8). In some embodiments, the antigen binding domain that specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of: SEQ ID NOs: 441, 442, 443, 444, 245 and 445 (OKT4); SEQ ID NOs: 446, 447, 448, 437, 308 and 449 (1.20); SEQ ID NOs: 450, 451, 448, 452, 313 and 449 (1.21); SEQ ID NOs: 450, 451, 448, 452, 308 and 449 (1.22); SEQ ID NOs: 453, 454, 455, 437, 255 and 456 (1.23); SEQ ID NOs: 457, 458, 459, 460, 255 and 404 (2.1) or SEQ ID NOs: 343, 350, 461, 347, 200 and 462 (2.8). In some embodiments, the antigen binding domain that specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of: SEQ ID NOs: 603, 604, 605, 606, 607 and 445 (OKT4); SEQ ID NOs: 608, 609, 610, 596, 611 and 449 (1.20); SEQ ID NOs: 612, 613, 610, 614, 615 and 449 (1.21); SEQ ID NOs: 612, 613, 610, 614, 611 and 449 (1.22); SEQ ID NOs: 616, 617, 618, 596, 550 and 456 (1.23); SEQ ID NOs: 619, 620, 621, 622, 597 and 404 (2.1) or SEQ ID NOs: 484, 623, 624, 487, 492 and 462 (2.8). In some embodiments, the antigen binding domain that specifically binds to CD4 D3 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 720 and 721 (OKT4); SEQ ID NOs: 722 and 723 (1.20); SEQ ID NOs: 724 and 725 (1.21); SEQ ID NOs: 724 and 726 (1.22); SEQ ID NOs: 727 and 728 (1.23); SEQ ID NOs: 727 and 729 (1.23 VL C36Y); SEQ ID NOs: 730 and 731 (2.1) or SEQ ID NOs: 732 and 733 (2.8). In some embodiments, the antibody or antigen-binding fragment thereof is insensitive to (i.e., binds to CD4 in the presence or absence of) the amino acid substitutions resulting from one or more of CD4 polymorphism variant IDs rs28919570 (R265W) and rs11064419 (F227S or F227C). In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120.

With respect to further embodiments of the CD4-targeted IL-15 molecule, in some embodiments, the antigen binding domain that specifically binds to CD4 binds to CD4 with an equilibrium dissociation constant (K_{D}) of lower than 1 nM, e.g., lower than 100 pM, lower than 90 pM, lower than 80 pM, lower than 70 pM, lower than 60 pM, lower than 50 pM, lower than 40 pM, or lower than 30 pM.

Further provided is a means for targeting IL-15 to CD4 D2 that is insensitive to (i.e., binds to CD4 in the presence and absence of) the amino acid substitution resulting from CD4 polymorphism variant ID rs11064416 (F123L). Further provided is a means for targeting IL-15 to one or both of CD4 D2 and D3 that is insensitive to (i.e., binds to CD4 in the presence or absence of) the amino acid substitutions resulting from one or more of CD4 polymorphism variant IDs rs28919570 (R265W), rs11064419 (F227S or F227C) and rs11064416 (F123L). Further provided is a means for targeting IL-15 to CD4 D3 that is insensitive to (i.e., binds to CD4 in the presence and absence of) the amino acid substitution resulting from CD4 polymorphism variant IDs rs28919570 (R265W) and rs11064419 (F227S or F227C). In some embodiments, the means specifically binds to CD4 with an equilibrium dissociation constant (KD) of lower than 1 nM, e.g., lower than 100 pM, lower than 90 pM, lower than 80 pM, lower than 70 pM, lower than 60 pM, lower than 50 pM, lower than 40 pM, or lower than 30 pM.

In some embodiments of the CD4-targeted IL-15 molecule, the fusion protein comprises an IL-15 receptor alpha subunit (IL15RA) SUSHI domain that binds to IL-15, is no longer than 65 amino acids, and comprises the amino acid sequence of SEQ ID NO: 734, or an amino acid sequence at least 90%, 91%, 92%, 93%, 94%, 95%, 96%, 97%, 98%, 99% identical to SEQ ID NO: 734. In some embodiments, the IL15RA SUSHI domain comprises the amino acid sequence of SEQ ID NO: 735, or an amino acid sequence at least 90%, 91%, 92%, 93%, 94%, 95%, 96%, 97%, 98%, 99% identical to SEQ ID NO: 735. In some embodiments, the IL15RA SUSHI domain comprises the amino acid sequence of any one of SEQ ID NOs: 736-739.

In some embodiments of the CD4-targeted IL-15v molecule, the fusion protein comprises an IL-15 variant comprising one or more of the following amino acid substitutions: N1D, N4D, S7G, D8N, D30N, D61N, E64Q, N65A, N65D, I68A, N71L, N71E, N72D, N77L, N79G, N79P, Q108E, N112A, N112G, N112D, N112S or N112K; or one of the following combinations of amino acid substitutions: N1D, N4D and D8N; N1D and D61N; N1D and E64Q; N1D and N65D; N4D and D61N; N4D and E64Q; N4D and N65D; S7G and N65D; D8G and D61N; D8G and E64Q; D8G and N65D; D8N and D61N; D8N and E64Q D8N and N65D; D30N and N65D; D61N and E64Q; D61N and N65D; E64Q and N65D; E64Q and Q108E; N65A and I68A; N65D and 168A; N71L and N79P; N71L and N112D; N79P and N112D; S7G, N65A and 168A; S7G, N65D and 168A; D30N, E64Q and N65D; D61N, E64Q and N65D; D61N, N65D and 168A; N71Q, N79Q and N112Q; N71A, N79A and N112A; N71G, N79G and N112G; N71S, N79L and N112E; N71E, N79P and N112K; N71L, N79P and N112K; N71L, N79P and N112D; N71K, N79P and N112D; N71E, N79P and N112S; N71E, N79P and N112D; N1D, D61N, E64Q and Q108E; N4D, D6IN, E64Q and Q108E; N71Q, N77L, N79Q and N112Q; N71A, N77L, N79A and N112A; N71G, N77L, N79G and N112G; N71S, N77L, N79L and N112E; N71E, N77L, N79P and N112K; N7IL, N77L, N79P and N112K; N71E, N77L, N79P and N112S; N71E, N77L, N79P and N112D; S7G, N65D, N71L, N79P and N112D; S7G, N65D, N71E, N77L, N79P and N112D; S7G, N65D, N71E, N77L, N79P and N112S; S7G, N65D, N71L, N77L, N79P and N112K; or S7G, N65D, N71E, N77L, N79P and N112K; wherein the position numbers are with respect to SEQ ID NO: 740 (wild-type IL-15). In some embodiments, the fusion protein comprises an IL-15 variant comprising one or more of the following amino acid substitutions: S7G and N65D; N65D and I68A; S7G, N65D and 168A; S7G, N65D, N71L, N79P and N112D; S7G, N65D, N71E, N77L, N79P and N112D; S7G, N65D, N71E, N77L, N79P and N112S; S7G, N65D, N71L, N77L, N79P and N112K; or S7G, N65D, N71E, N77L, N79P, N112K; wherein the position numbers are with respect to SEQ ID NO: 740. In some embodiments, the CD4-targeted IL-15v molecule comprises an IL-15 or IL-15v of any one of SEQ ID NOs: 740-813, 1124 and 1125. In some embodiments, the CD4-targeted IL-15v molecule comprises an IL-15 or IL-15v of any one of SEQ ID NOs: 743, 750-752, 754-756, 758, 768-771, 774, 777, 779, 783-788, 790-798, 801-808, 810-813 and 1125.

In some embodiments of the CD4-targeted IL-15v molecule, the fusion protein comprises in sequential order from N-terminus to C-terminus, (i) the IL15RA SUSHI domain, (ii) the IL-15 or variant thereof, and (iii) Fc domain. In some embodiments, the fusion protein comprises a flexible linker between the IL15RA SUSHI domain and the IL-15 or variant thereof. In some embodiments the linker has a length of from about 4 to about 50 amino acids, e.g., from about 5 amino acids to about 25 amino acids, e.g., from about 15 amino acids to about 25 amino acids. In some embodiments, the linker comprises from 1 to 10 units, e.g., 1 to 5 units, e.g., 3 to 5 units, of a poly-glycine serine linker selected from GGGS, GGGGS and combinations thereof. In some embodiments, the linker comprises 5 units of GGGGS (GGGGSGGGGSGGGGSGGGGSGGGGS; SEQ ID NO: 1085).

Structurally, in some embodiments of the CD4-targeted IL-15 molecule, the fusion protein comprises: (i) an IL-15 receptor alpha subunit (IL15RA) SUSHI domain, (ii) an IL-15 or variant thereof (IL-15v) of any one of SEQ ID NOs: 814-887, 1126 and 1127. In some embodiments, the fusion protein comprises: (i) an IL-15 receptor alpha subunit (IL15RA) SUSHI domain, (ii) an IL-15 or variant thereof (IL-15v) of any one of SEQ ID NOs: 817, 824-826, 828-830, 832, 842-845, 848, 851, 853, 857-862, 864-872, 875-882, 884-887 and 1127. Functionally, in some embodiments, the IL-15v or the IL-15RA SUSHI domain-IL-15v fusion protein binds to IL-2Rβ with a binding equilibrium dissociation constant (K_{D}) of at least 1 μM, e.g., a K_{D} of at least 1.5 μM, at least 2 μM, at least 2.5 μM, at least 3 μM, at least 3.5 μM, at least 4 μM, at least 4.5 μM or at least 5 μM. In some embodiments, the IL-15v or the IL-15RA SUSHI domain-IL-15v fusion protein binds to IL-2Rβγ with a K_{D} of at least 1 μM, e.g., a K_{D} of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM. In some embodiments, the IL-15v or the IL-15RA SUSHI domain-IL-15v fusion protein binds to IL-2Rβγ with a K_{D} that is at least 1000-fold to the K_{D} of the antigen binding domain specifically binding to CD4. In some embodiments, the IL-15v or the IL-15RA SUSHI domain-IL-15v fusion protein binds to IL-2Rβγ with a binding equilibrium dissociation constant (K_{D}) of at least 1 μM, e.g., a K_{D} of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM, and the antigen binding domain that specifically binds to CD4 binds to CD4 with a K_{D} of lower than 1 nM, e.g., lower than 100 pM, lower than 90 pM, lower than 80 pM, lower than 70 pM, lower than 60 pM, lower than 50 pM, lower than 40 pM, or lower than 30 pM. In some embodiments, the IL-15v or the IL-15RA SUSHI domain-IL-15v fusion protein induces CD4+ T cell proliferation with an EC50 of less than 5 nM, e.g., less than 4 nM, less than 3 nM, less than 2 nM, less than 1 nM, e.g., wherein CD4+ T cell proliferation potency is measured by marker of proliferation Ki-67 activation (MK167; NCBI Gene ID: 4288). In some embodiments, the IL-15v or the IL-15RA SUSHI domain-IL-15v fusion protein activates both naïve CD4+ T cells and memory CD4+ T cells. In some embodiments, the IL-15v or the IL-15RA SUSHI domain-IL-15v fusion protein induces CD8+ T cells and/or natural killer (NK) cell proliferation with an EC50 of greater than 100 nM, e.g., wherein CD8+ T cell and/or NK cell proliferation potency is measured by Ki-67 activation.

In some embodiments of the CD4-targeted IL-15 molecule, the first Fc domain and the second Fc domain are human IgG1. In some embodiments, one or both of the first Fc domain and the second Fc domain comprises one or more amino acid substitutions in the Fc region at a residue position selected from the group consisting of: L234A, L234V, L234F, L235A, L235E, G237A, P331S, and any combination thereof, wherein the numbering of the residues is according to Eu numbering. In some embodiments, the first Fc domain and the second Fc domain are human IgG4. In some embodiments, one or both of the first Fc domain and the second Fc domain comprises one or more amino acid substitutions in the Fc region at a residue position selected from the group consisting of: F234V, F234A, L235A, L235E, G237A, S228P, and any combination thereof, wherein the numbering of the residues is according to Eu numbering. In some embodiments, the first and second Fc domain comprise amino acid substitutions to facilitate heterodimerization. In some embodiments, the first Fc domain and the second Fc domain comprise the following amino acid substitutions (Eu numbering), respectively: T366W and T366S/L368A/Y407V; T366S/L368A/Y407V and T366W; T366W/S354C and T366S/L368A/Y407V/Y349C; T366S/L368A/Y407V/Y349C and T366W/S354C; S364H/F405A and Y349T/T394; Y349T/T394 and S364H/F405A; T350V/L351Y/F405A/Y407V and T350V/T366L/K392L/T394W; T350V/T366L/K392L/T394W and T350V/L351Y/F405A/Y407V; K360D/D399M/Y407A and E345R/Q347R/T366V/K409V; E345R/Q347R/T366V/K409V and K360D/D399M/Y407A; K409D/K392D and D399K/E356K; D399K/E356K and K409D/K392D; K360E/K409W and Q347R/D399V/F405T; Q347R/D399V/F405T and K360E/K409W; K360E/K409W/Y349C and Q347R/D399V/F405T/S354C; Q347R/D399V/F405T/S354C and K360E/K409W/Y349C; K370E/K409W and E357N/D399V/F405T; or E357N/D399V/F405T and K370E/K409W. In some embodiments, neither of the first nor second Fc domain comprise amino acid substitutions to extend serum half-life. In some embodiments, one or both of the first and second Fc domain comprise amino acid substitutions to extend serum half-life. In some embodiments, one or both of the first Fc domain and the second Fc domain comprise the following amino acids at the indicated positions (EU index numbering): tyrosine at position 252, threonine at position 254 and glutamic acid at position 256 (252Y, 254T and 256E); leucine at position 428 (428L); glutamine at position 250 and leucine at position 428 (250Q and 428L); leucine at position 428 and serine at position 434 (428L and 434S); leucine at position 428 and alanine at position 434 (428L and 434A); arginine at position 311 and leucine at position 428 (311R and 428L); glycine at position 309 and leucine at position 428 (309G and 428L); glutamine at position 307, valine at position 311 and valine at position 378 (307Q, 311V and 378V); or aspartic acid at position 256, aspartic acid at position 286, arginine at position 307, valine at position 311 and valine at position 378 (256D, 286D, 307R, 311V and 378V). In some embodiments, one of the first or the second Fc domain comprise amino acid substitutions to facilitate purification. In some embodiments, one of the first Fc domain or the second Fc domain comprise the following amino acids at the indicated positions (EU index numbering): one or both of an arginine at position 435 (H435R) and a phenylalanine or an alanine at position 436 (Y436F or Y436A).

In some embodiments, the CD4-targeted IL-15v molecule comprises a heavy chain (HC1) and a light chain (LC1) that bind to CD4 D1 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 921, 922 and 923 (100); SEQ ID NOs: 924, 922 and 925 (101); SEQ ID NOs: 926, 922 and 925 (102); SEQ ID NOs: 926, 922 and 1074 (103); SEQ ID NOs: 926, 922 and 1075 (104); SEQ ID NOs: 926, 922 and 1076 (105); SEQ ID NOs: 926, 922 and 974 (106); SEQ ID NOs: 926, 929 and 1076 (107); SEQ ID NOs: 927, 928 and 925 (108); SEQ ID NOs: 926, 929 and 925 (109); SEQ ID NOs: 926, 922 and 985 (110); SEQ ID NOs: 930, 922 and 923 (111); SEQ ID NOs: 931, 922 and 925 (112); SEQ ID NOs: 932, 922 and 933 (113); SEQ ID NOs: 934, 922 and 935 (114); SEQ ID NOs: 936, 937 and 923 (115); SEQ ID NOs: 938, 939 and 923 (116); SEQ ID NOs: 940, 941 and 923 (117); SEQ ID NOs: 942, 943 and 923 (118); SEQ ID NOs: 944, 945 and 923 (119); SEQ ID NOs: 946, 947 and 923 (120); SEQ ID NOs: 948, 949 and 923 (121); SEQ ID NOs: 950, 951 and 923 (122); SEQ ID NOs: 952, 953 and 923 (123); SEQ ID NOs: 954, 955 and 923 (124); SEQ ID NOs: 956, 955 and 923 (125); SEQ ID NOs: 957, 958 and 923 (126); SEQ ID NOs: 959, 960 and 923 (127); SEQ ID NOs: 961, 962 and 923 (128); SEQ ID NOs: 961, 960 and 923 (129); SEQ ID NOs: 926, 922 and 1091 (249); SEQ ID NOs: 926, 922 and 1092 (251); SEQ ID NOs: 926, 922 and 1093 (252); SEQ ID NOs: 926, 922 and 1094 (253); SEQ ID NOs: 926, 922 and 1095 (254); SEQ ID NOs: 926, 922 and 1096 (255); SEQ ID NOs: 926, 922 and 1097 (259); SEQ ID NOs: 926, 922 and 1098 (260); SEQ ID NOs: 926, 922 and 1099 (261) or SEQ ID NOs: 926, 922 and 1100 (262).

In some embodiments, the CD4-targeted IL-15v molecule comprises a heavy chain (HC1) and a light chain (LC1) that bind to CD4 D2 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 1086, 1087 and 1088 (245); SEQ ID NOs: 963, 964 and 923 (130); SEQ ID NOs: 965, 966 and 923 (131); SEQ ID NOs: 967, 968 and 923 (132); SEQ ID NOs: 969, 970 and 923 (133); SEQ ID NOs: 969, 970 and 971 (134); SEQ ID NOs: 969, 970 and 972 (135); SEQ ID NOs: 973, 970 and 974 (136); SEQ ID NOs: 975, 976 and 923 (137); SEQ ID NOs: 977, 978 and 923 (138); SEQ ID NOs: 977, 978 and 971 (139); SEQ ID NOs: 977, 978 and 972 (140); SEQ ID NOs: 977, 979 and 972 (141); SEQ ID NOs: 980, 979 and 974 (142); SEQ ID NOs: 981, 982 and 923 (143); SEQ ID NOs: 981, 982 and 971 (144); SEQ ID NOs: 981, 982 and 972 (145); SEQ ID NOs: 981, 983 and 972 (146); SEQ ID NOs: 984, 983 and 974 (147); SEQ ID NOs: 984, 983 and 985 (148); SEQ ID NOs: 986, 987 and 923 (149); SEQ ID NOs: 988, 989 and 923 (150); SEQ ID NOs: 988, 989 and 971 (151); SEQ ID NOs: 988, 989 and 972 (152); SEQ ID NOs: 990, 991 and 923 (153); SEQ ID NOs: 990, 991 and 971 (154); SEQ ID NOs: 990, 991 and 972 (155) or SEQ ID NOs: 992, 991 and 974 (156).

In some embodiments, the CD4-targeted IL-15v molecule comprises a heavy chain (HC1) and a light chain (LC1) that bind to one or both of CD4 D2 and CD4 D3 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 993, 994 and 923 (157); SEQ ID NOs: 995, 996 and 923 (158); SEQ ID NOs: 997, 998 and 923 (159); SEQ ID NOs: 999, 1000 and 923 (160); SEQ ID NOs: 1001, 1002 and 923 (161); SEQ ID NOs: 1003, 1004 and 923 (162); SEQ ID NOs: 1005, 1006 and 923 (163) or SEQ ID NOs: 1007, 1008 and 923 (164).

In some embodiments, the CD4-targeted IL-15v molecule comprises a heavy chain (HC1) and a light chain (LC1) that bind to CD4 D3 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 1089, 1090 and 1088 (246); SEQ ID NOs: 1009, 1010 and 923 (165); SEQ ID NOs: 1009, 1010 and 971 (166); SEQ ID NOs: 1009, 1010 and 972 (167); SEQ ID NOs: 1011, 1012 and 923 (168); SEQ ID NOs: 1011, 1012 and 971 (169); SEQ ID NOs: 1011, 1012 and 972 (170); SEQ ID NOs: 1013, 1012 and 974 (171); SEQ ID NOs: 1011, 1014 and 923 (172); SEQ ID NOs: 1011, 1014 and 971 (173); SEQ ID NOs: 1011, 1014 and 972 (174); SEQ ID NOs: 1013, 1014 and 974 (175); SEQ ID NOs: 1015, 1016 and 923 (176); SEQ ID NOs: 1015, 1016 and 971 (177); SEQ ID NOs: 1015, 1017 and 971 (178); SEQ ID NOs: 1015, 1016 and 972 (179); SEQ ID NOs: 1015, 1017 and 972 (180); SEQ ID NOs: 1018, 1017 and 974 (181); SEQ ID NOs: 1018, 1017 and 985 (182); SEQ ID NOs: 1019, 1020 and 923 (183); SEQ ID NOs: 1019, 1020 and 971 (184); SEQ ID NOs: 1019, 1020 and 972 (185); SEQ ID NOs: 1021, 1020 and 974 (186); SEQ ID NOs: 1022, 1023 and 923 (187); SEQ ID NOs: 1024, 1025 and 923 (188); SEQ ID NOs: 1011, 1014 and 1026 (189); SEQ ID NOs: 1027, 1014 and 1028 (190); SEQ ID NOs: 1027, 1014 and 1029 (191); SEQ ID NOs: 1011, 1014 and 1030 (192); SEQ ID NOs: 1011, 1014 and 1031 (193); SEQ ID NOs: 1011, 1014 and 1032 (194); SEQ ID NOs: 1011, 1014 and 1077 (220); SEQ ID NOs: 1011, 1014 and 1033 (195); SEQ ID NOs: 1011, 1014 and 1034 (196); SEQ ID NOs: 1011, 1014 and 1035 (197); SEQ ID NOs: 1011, 1014 and 1036 (198); SEQ ID NOs: 1011, 1014 and 1037 (199); SEQ ID NOs: 1011, 1014 and 1038 (200); SEQ ID NOs: 1011, 1014 and 1039 (201); SEQ ID NOs: 1011, 1014 and 1040 (202); SEQ ID NOs: 1011, 1014 and 1041 (203); SEQ ID NOs: 1011, 1014 and 1042 (204); SEQ ID NOs: 1011, 1014 and 1043 (205); SEQ ID NOs: 1011, 1014 and 1044 (206); SEQ ID NOs: 1011, 1014 and 1045 (207); SEQ ID NOs: 1011, 1014 and 1046 (208); SEQ ID NOs: 1011, 1014 and 1047 (209); SEQ ID NOs: 1011, 1014 and 1048 (210); SEQ ID NOs: 1011, 1014 and 1049 (211); SEQ ID NOs: 1011, 1014 and 1050 (212); SEQ ID NOs: 1011, 1014 and 1051 (213); SEQ ID NOS: 1011, 1014 and 1052 (214); SEQ ID NOs: 1011, 1014 and 1053 (215); SEQ ID NOs: 1011, 1014 and 1054 (216); SEQ ID NOs: 1011, 1014 and 1055 (217) or SEQ ID NOs: 1011, 1014 and 1056 (218).

In some embodiments, the CD4-targeted IL-15v molecule comprises a heavy chain (HC1) and a light chain (LC1) that bind to CD4 D3 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1057 (219). In some embodiments, the CD4-targeted IL-15v molecule comprises a heavy chain (HC1) and a light chain (LC1) that bind to CD4 D3 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 1011, 1014 and 923 (172); SEQ ID NOs: 1011, 1014 and 971 (173); SEQ ID NOs: 1011, 1014 and 972 (174); SEQ ID NOs: 1011, 1014 and 1026 (189); SEQ ID NOs: 1011, 1014 and 1030 (192); SEQ ID NOs: 1011, 1014 and 1031 (193); SEQ ID NOs: 1011, 1014 and 1032 (194); SEQ ID NOs: 1011, 1014 and 1077 (220); SEQ ID NOs: 1011, 1014 and 1033 (195); SEQ ID NOs: 1011, 1014 and 1034 (196); SEQ ID NOs: 1011, 1014 and 1035 (197); SEQ ID NOs: 1011, 1014 and 1036 (198); SEQ ID NOs: 1011, 1014 and 1037 (199); SEQ ID NOs: 1011, 1014 and 1038 (200); SEQ ID NOs: 1011, 1014 and 1039 (201); SEQ ID NOs: 1011, 1014 and 1040 (202); SEQ ID NOs: 1011, 1014 and 1041 (203); SEQ ID NOs: 1011, 1014 and 1042 (204); SEQ ID NOs: 1011, 1014 and 1043 (205); SEQ ID NOs: 1011, 1014 and 1044 (206); SEQ ID NOs: 1011, 1014 and 1045 (207); SEQ ID NOs: 1011, 1014 and 1046 (208); SEQ ID NOs: 1011, 1014 and 1047 (209); SEQ ID NOs: 1011, 1014 and 1048 (210); SEQ ID NOs: 1011, 1014 and 1049 (211); SEQ ID NOs: 1011, 1014 and 1050 (212); SEQ ID NOs: 1011, 1014 and 1051 (213); SEQ ID NOs: 1011, 1014 and 1052 (214); SEQ ID NOs: 1011, 1014 and 1053 (215); SEQ ID NOs: 1011, 1014 and 1054 (216); SEQ ID NOs: 1011, 1014 and 1055 (217) or SEQ ID NOs: 1011, 1014 and 1056 (218) (antibody SCT 1.22).

In some embodiments, the CD4-targeted IL-15v molecule comprises a heavy chain (HC1) and a light chain (LC1) that bind to Rhesus CD4 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in: SEQ ID NOs: 1101, 1102 and 1103 (247), SEQ ID NOs: 1058, 1059 and 1060 (221) or SEQ ID NOs: 1221, 1222 and 1223 (277).

With respect to further embodiments of the CD4-targeted IL-15v molecules, in some embodiments, the CD4-targeted IL-15v has a serum half-life in a human or a non-human primate of less than 24 hours, e.g., less than 20 hours, less than 18 hours, less than 16 hours.

Provided is a polynucleotide or multiple polynucleotides encoding the IL-15v, the fusion protein, the CAR, the antibody or antigen-binding fragment, or the CD4-targeted IL-15v molecule as described above and herein. In some embodiments, the polynucleotide or polynucleotides encode the heavy chain comprising the antigen binding domain that specifically binds to CD4 and comprise a nucleic acid sequence selected from the group consisting of SEQ ID NOs: 1104, 1107, 1109, 1112, 1115, 1118 and 1228, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1104, 1107, 1109, 1112, 1115, 1118 and 1228. In some embodiments, the polynucleotide or polynucleotides encode the light chain comprising the antigen binding domain that specifically binds to CD4 and comprise a nucleic acid sequence selected from the group consisting of SEQ ID NOs: 1105, 1110, 1113, 1116 and 1229, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1105, 1110, 1113, 1116 and 1229. In some embodiments, the polynucleotide or polynucleotides encode the IL15RASushi-IL15v-Fc fusion protein and comprise a nucleic acid sequence selected from the group consisting of SEQ ID NOs: 1106, 1108, 1111, 1114, 1117, 1119 and 1230, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1106, 1108, 1111, 1114, 1117, 1119 and 1230. In some embodiments, the polynucleotide or polynucleotides encode the heavy chain comprising the antigen binding domain that specifically binds to CD4, the light chain comprising the antigen binding domain that specifically binds to CD4 and the IL15RASushi-IL15v-Fc fusion protein of a CD4-targeted IL-15v, as described herein, and comprise the following polynucleotide sequences, or polynucleotide sequences that are at least 80%, at least 85%, at least 90%, at least 91%, at least 92%, at least 93%, at least 94%, at least 95%, at least 96%, at least 97%, at least 98%, or at least 99% identical to the polynucleotide sequences set forth, respectively, in: SEQ ID NOs: 1104, 1105 and 1106; SEQ ID NOs: 1107, 1105 and 1108; SEQ ID NOs: 1109, 1110 and 1111; SEQ ID NOs: 1112, 1113 and 1114; SEQ ID NOs: 1115, 1116 and 1117; SEQ ID NOs: 1118, 1113 and 1119; or SEQ ID NOs: 1228, 1229 and 1230. In some embodiments, the polynucleotide or polynucleotides are DNA, cDNA, RNA or mRNA. Further provided is an expression cassette or multiple expression cassettes comprising one or more regulatory sequences operably linked to the polynucleotide or polynucleotides as described above and herein. Further provided is a vector comprising the polynucleotide or polynucleotides or the expression cassette as described herein. In some embodiments, the vector is a plasmid vector or a viral vector. In some embodiments, the viral vector comprises a DNA virus or an RNA virus. In some embodiments, the viral vector is from a viral family selected from the group consisting of: Adenoviridae (e.g., Adenovirus), Arenaviridae (e.g., lymphocytic choriomeningitis mammarenavirus, Cali mammarenavirus (a.k.a., Pichinde mammarenavirus), Poxviridae (e.g., Vaccinia virus), Herpesviridae (e.g., Herpesvirus, e.g., HSV-1), Parvoviridae (e.g., Parvovirus H1), Reoviridae (e.g., Reovirus), Retroviridae (e.g., Lentivirus), Picornaviridae (e.g., Coxsackievirus, Seneca Valley Virus, Poliovirus), Paramyxoviridae (e.g., Measles virus, Newcastle disease virus (NDV)), Rhabdoviridae (e.g., Vesicular stomatitis virus (VSV)), Togaviridae (e.g., Alphavirus, Sindbis virus) and Enteroviridae (e.g., Echovirus). Further provided is a lipoplex, e.g., lipid nanoparticle (LNP), comprising the polynucleotide or polynucleotides, the expression cassette, or the vector as described herein. In some embodiments of the lipoplex, e.g., lipid nanoparticle (LNP), the polynucleotide or polynucleotides are mRNA.

Further provided is a cell or population of cells comprising the polynucleotide or polynucleotides, the expression cassette, or the vector, as described herein, wherein the cell or population of cells expresses the IL-15v, the IL-15RA SUSHI domain-IL-15v fusion protein or the CD4-targeted IL-15 molecule, as described herein. In some embodiments, the cell or population of cells is a eukaryotic cell. In some embodiments, the cell or population of cells comprises a mammalian cell, an insect cell, a plant cell or a yeast cell. In some embodiments, the mammalian cell is a Chinese Hamster Ovary (CHO) cell. In some embodiments, the mammalian cell is a human cell. In some embodiments, the cell is a human embryonic kidney cell. In some embodiments, the population of cells produces at least 4 g/L, e.g., at least 5 g/L, at least 6 g/L, at least 7 g/L, at least 8 g/L, at least 9 g/L, or more, CD4-targeted IL-15 molecule.

Further provided is a method of producing a CD4-targeted IL-15 molecule. In some embodiments, the method comprises: (a) culturing a cell or population of cells, described above and herein, transformed with the polynucleotide or polynucleotides, or the expression cassette or multiple expression cassettes, or the vector, described above and herein, in a cell culture under conditions sufficient to express the CD4-targeted IL-15 molecules; and (b) isolating or purifying the CD4-targeted IL-15 molecules from the cell culture. In some embodiments, the polypeptide comprising the antigen binding domain that specifically binds to CD4 and the polypeptide comprising the IL15RASushi-IL15v-Fc fusion protein are expressed and assembled in the same cell. In some embodiments, the isolating or purifying step comprises Protein A chromatography. In some embodiments, at least about 90%, 91%, 92%, 93%, 94%, 95%, 96%, 97%, 98%, 99%, or more, of the CD4-targeted IL-15 molecules are isolated or purified. In some embodiments, the cell or population of cells are cultured in a culture volume of at least 2 L, e.g., at least 5 L, 10 L, 50 L, 100 L, 150 L, 200 L, 250 L, 500 L, 1000 L, 2000 L, or more. In some embodiments, the population of cells produces at least 4 g/L, e.g., at least 5 g/L, at least 6 g/L, at least 7 g/L, at least 8 g/L, at least 9 g/L, or more, of the CD4-targeted IL-15 molecule. In some embodiments, the method further comprises formulating the CD4-targeted IL-15 molecules into a sterile pharmaceutical composition suitable for administration to a human subject.

Further provided is a pharmaceutical composition comprising the CD4-targeted IL-15 molecule, the polynucleotide or polynucleotides, the expression cassette or multiple expression cassettes, the vector, or the lipoplex (e.g., LNP), described above and herein, and a pharmaceutically acceptable carrier. In some embodiments, the composition comprises an aqueous formulation. In some embodiments, the composition is lyophilized. In some embodiments, the pharmaceutical composition is formulated for intravenous, subcutaneous, intramuscular, intradermal, or mucosal (e.g. buccal, intranasal, intrarectal, intravaginal) administration. In some embodiments, the pharmaceutical composition further comprises one or more additional therapeutic agents. In some embodiments, the pharmaceutical composition further comprises a second additional therapeutic agent. In some embodiments, the pharmaceutical composition further comprises second and third therapeutic agents. In some embodiments, the pharmaceutical composition further comprises a latency reversal agent (LRA). In some embodiments, the pharmaceutical composition further comprises a toll-like receptor (TLR) agonist. In some embodiments, the pharmaceutical composition further comprises a TLR agonist selected from a TLR2 agonist, a TLR3 agonist, a TLR7 agonist, a TLR8 agonist or a TLR9 agonist. In some embodiments, the TLR3 agonist is selected from the group consisting of rintatolimod and Polyinosinic-Polycytidylic Acid Stabilized with Polylysine and Carboxymethylcellulose (poly ICLC (HILTONOL®)). In some embodiments, the TLR7 agonist is selected from the group consisting of vesatolimod, imiquimod, resiquimod and NKTR-262. In some embodiments, the TLR8 agonist is selected from the group consisting of selgantolimod, motolimod, resiquimod and NKTR-262. In some embodiments, the TLR9 agonist is selected from the group consisting of cavrotolimod, cobitolimod, agatolimod, leftolimod, litenimod and tilsotolimod. In some embodiments, the pharmaceutical composition further comprises a multispecific T-cell engager, e.g., a bispecific T-cell engager. In some embodiments, the pharmaceutical composition further comprises a CD4-Fc fusion protein. In some embodiments, the pharmaceutical composition further comprises GS 8588. In some embodiments, the pharmaceutical composition further comprises a lipid nanoparticle (LNP) comprising a human immunodeficiency virus (HIV) tat mRNA. In some embodiments, the pharmaceutical composition further comprises a non-nucleoside reverse transcriptase inhibitor (NNRTI) as targeted activator of cell kill (TACK). In some embodiments, the TACK is selected from efavirenz (EFV), rilpivirine (RPV), pyrimidone pyr01, and pyrimidone pyr02. In some embodiments, the pharmaceutical composition further comprises a Second Mitochondria-derived Activator of Caspase (SMAC) mimetic or an inhibitor of cellular inhibitor of apoptosis proteins (cIAP). In some embodiments, the SMAC mimetic or cIAP inhibitor is selected from xevinapant, dasminapant, birinapant, tolinapant lactate, ciapavir and AZD5582. In some embodiments, the pharmaceutical composition further comprises an inhibitor of phosphatase and tensin homolog (PTEN inhibitor. In some embodiments, the pharmaceutical composition further comprises an inhibitor of one or both of protein tyrosine phosphatase non-receptor type 1 (PTPN1) and protein tyrosine phosphatase non-receptor type 2 (PTPN2). In some embodiments, the inhibitor of one or both of PTPN1 and PTPN2 is selected from 3-Hydroxy-1,2,3-benzotriazin-4(3H)-one (HODHBt), avarol, osunprotafib, trodusquemine, S nitrosoglutathione, S-nitroso-N-acetylpenicillamine, talaramide Q, aurothiomalate disodium, daiokanzoto, berberine chloride, and alpha-tocopherolquinone. In some embodiments, the pharmaceutical composition further comprises one or more anti-HIV vaccines. In some embodiments, the anti-HIV vaccine is a viral vector vaccine. In some embodiments, the viral vector is selected from an arenavirus vector, a modified vaccinia virus Ankara (MVA)) vector, and a simian (e.g., chimpanzee, gorilla, rhesus) adenovirus vector. In some embodiments, the pharmaceutical composition further comprises one or more anti-HIV broadly neutralizing antibodies, e.g., that bind to different epitopes or regions of gp120 selected from the group consisting of: (i) third variable loop (V3) (e.g., high mannose patch) comprising a N332 oligomannose glycan; (ii) second variable loop (V2) (e.g., Env trimer apex); (iii) CD4 binding site (CD4bs); (iv) gp120/gp41 interface; or (v) silent face of gp120. In some embodiments, the pharmaceutical composition further comprises an anti-HIV broadly neutralizing antibody that binds to the third variable loop (V3) (e.g., high mannose patch) comprising a N332 oligomannose glycan and the second antigen binding molecule binds to the CD4 binding site (CD4bs). In some embodiments, the pharmaceutical composition further comprises an anti-HIV broadly neutralizing antibody that competes with or comprises VH and VL regions from an antibody selected from the group consisting of zinlirvimab (GS-2872), elipovimab (GS-9722), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the pharmaceutical composition further comprises an anti-HIV broadly neutralizing antibody that competes with or comprises VH and VL regions from an antibody selected from the group consisting of zinlirvimab (GS-2872), 10-1074, 10-1074-J, elipovimab (GS-9722), PGT-121, PGT-121.66, PGT-121.414, PGT-128 and PGT-134. In some embodiments, the pharmaceutical composition further comprises an anti-HIV broadly neutralizing antibody that competes with or comprises VH and VL regions from an antibody selected from the group consisting of teropavimab (GS-5423), GS 9723, 3BNC117, 3BNC60, b12, F105, VRC01, VRC07, VRC07-523, VRC03, VRC06, VRC06b01 VRC08, VRC0801, NIH45-46, PGV04 (VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25. In some embodiments, the pharmaceutical composition further comprises an anti-HIV broadly neutralizing antibody that competes with or comprises VH and VL regions from an antibody selected from the group consisting of teropavimab (GS-5423), GS 9723, 3BNC117, VRC07 and VRC07-523. In some embodiments, the pharmaceutical composition further comprises an anti-HIV broadly neutralizing antibody that competes with or comprises VH and VL regions from an antibody that binds to the gp120 second variable loop (V2) (e.g., Env trimer apex). In some embodiments, the pharmaceutical composition further comprises an anti-HIV broadly neutralizing antibody that competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the pharmaceutical composition further comprises an anti-HIV broadly neutralizing antibody that competes with or comprises VH and VL regions from an antibody that binds to the gp120/gp41 interface. In some embodiments, the pharmaceutical composition further comprises an anti-HIV broadly neutralizing antibody that competes with or comprises VH and VL regions from an antibody selected from the group consisting of PGT-151, CAP248-2B, 35022, 8ANC195, ACS202, VRC34 and VRC34.01. In some embodiments, the pharmaceutical composition further comprises an anti-HIV broadly neutralizing antibody that competes with or comprises VH and VL regions from an antibody that binds to an epitope or region of gp41 in the membrane proximal region (MPER). In some embodiments, the pharmaceutical composition further comprises an anti-HIV broadly neutralizing antibody that competes with or comprises VH and VL regions from an antibody selected from the group consisting of 10E8, 10E8v4, 10E8-5R-100cF, 4E10, DH511.11P, 2F5, A32, 7b2, and LN01.

Further provided is a method of inducing, stimulating or promoting the proliferation of CD4+ T cells. In some embodiments, the method comprises contacting the CD4+ T cells with an effective amount of the CD4-targeted IL-15 molecule, the means for targeting IL-15v to CD4, or the pharmaceutical composition, as described above and herein. In some embodiments, the CD4+ T cells are in vivo. In some embodiments, the CD4+ T cells are in vitro. In some embodiments, the CD4+ T cell proliferation is induced, stimulated or promoted with a potency that is at least 100-fold, e.g., at least 200-fold, at least 300-fold, at least 400-fold, at least 500-fold, at least 600-fold, or more, in comparison to the potency for inducing, stimulating or promoting CD8+ T cell or NK cell proliferation. In some embodiments, the cell proliferation potency is measured by Ki-67 activation.

Further provided is a method or use of activating a latent viral reservoir in a subject infected with human immunodeficiency virus (HIV). Further provided is a method or use of treating or preventing human immunodeficiency virus (HIV) in a subject in need thereof. In some embodiments, the method or use comprises administering to a subject a therapeutically effective amount of the CD4-targeted IL-15 molecule, the means for targeting IL-15v to CD4, the polynucleotide or polynucleotides, the expression cassette, the vector, the lipoplex (e.g, LNP), or the pharmaceutical composition, as described above and herein. In some embodiments, the method or use further comprises administering to the subject one or more additional therapeutic agents. In some embodiments, the method or use further comprises administering an HIV latency reversing agent (LRA). In some embodiments, the method or use further comprises administering a toll-like receptor (TLR) agonist. In some embodiments, the method or use further comprises administering a TLR agonist selected from a TLR2 agonist, a TLR3 agonist, a TLR7 agonist, a TLR8 agonist or a TLR9 agonist. In some embodiments, the TLR3 agonist is selected from the group consisting of rintatolimod and Polyinosinic-Polycytidylic Acid Stabilized with Polylysine and Carboxymethylcellulose (poly ICLC (HILTONOL®)). In some embodiments, the TLR7 agonist is selected from the group consisting of vesatolimod, imiquimod, resiquimod and NKTR-262. In some embodiments, the TLR8 agonist is selected from the group consisting of selgantolimod, motolimod resiquimod and NKTR-262. In some embodiments, the TLR9 agonist is selected from the group consisting of cavrotolimod, cobitolimod, agatolimod, leftolimod, litenimod and tilsotolimod. In some embodiments, the method further comprises administering a multispecific T-cell engager, e.g., a bispecific T-cell engager. In some embodiments, the method further comprises administering a CD4-Fc fusion protein. In some embodiments, the method further comprises administering GS 8588. In some embodiments, the method further comprises administering a lipid nanoparticle (LNP) comprising an HIV tat mRNA. In some embodiments, the method further comprises administering a non-nucleoside reverse transcriptase inhibitor (NNRTI) as targeted activator of cell kill (TACK). In some embodiments, the TACK is selected from efavirenz (EFV), rilpivirine (RPV), pyrimidone pyr01, and pyrimidone pyr02. In some embodiments, the method further comprises administering a Second Mitochondria-derived Activator of Caspase (SMAC) mimetic or an inhibitor of cellular inhibitor of apoptosis proteins (cIAP). In some embodiments, the SMAC mimetic or cIAP inhibitor is selected from xevinapant, dasminapant, birinapant, tolinapant lactate, ciapavir and AZD5582. In some embodiments, the method further comprises administering an inhibitor of one or both of protein tyrosine phosphatase non-receptor type 1 (PTPN1) and protein tyrosine phosphatase non-receptor type 2 (PTPN2). In some embodiments, the inhibitor of one or both of PTPN1 and PTPN2 is selected from 3-Hydroxy-1,2,3-benzotriazin-4(3H)-one (HODHBt), avarol, osunprotafib, trodusquemine, S nitrosoglutathione, S-nitroso-N-acetylpenicillamine, talaramide Q, aurothiomalate disodium, daiokanzoto, berberine chloride, and alpha-tocopherolquinone. In some embodiments, the method further comprises administering one or more anti-HIV vaccines. In some embodiments, the anti-HIV vaccine is a viral vector vaccine. In some embodiments, the viral vector is selected from an arenavirus vector, modified vaccinia virus Ankara (MVA)) vector, and a simian (e.g., chimpanzee, gorilla, rhesus) adenovirus vector. In some embodiments, the method further comprises administering an HIV vaccine in a prime-boost regimen, e.g., comprising: (a) Priming at a first time point by co-administering a first viral vector with the CD4-targeted IL-15 molecule; and (b) Boosting at a second time point by co-administering a second viral vector with the CD4-targeted IL-15 molecule. In some embodiments, the first viral vector and the second viral vector are the same or are different. In some embodiments, the method further comprises administering to the subject one or more anti-HIV broadly neutralizing antibodies. In some embodiments, the one or more anti-HIV broadly neutralizing antibodies bind to an epitope or region of gp120 selected from the group consisting of: (i) third variable loop (V3) and/or high mannose patch comprising a N332 oligomannose glycan; (ii) second variable loop (V2) and/or Env trimer apex; (iii) CD4 binding site (CD4bs); (iv) gp120/gp41 interface; or (v) silent face of gp120. In some embodiments, the method further comprises administering to the subject the one or more anti-HIV broadly neutralizing antibodies that bind to an epitope or region of gp120 in the third variable loop (V3) and/or high mannose patch comprising a N332 oligomannose glycan and competes with or comprises VH and VL regions from an antibody selected from the group consisting of zinlirvimab, GS-9722, PGT-121, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the method further comprises administering to the subject one or more anti-HIV broadly neutralizing antibodies that bind to an epitope or region of gp120 in the second variable loop (V2) and/or Env trimer apex and competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the method further comprises administering to the subject one or more anti-HIV broadly neutralizing antibodies that bind to an epitope or region of gp120 in the CD4 binding site (CD4bs) and competes with or comprises VH and VL regions from an antibody selected from the group consisting of teropavimab (GS-5423), 3BNC117, GS-9723, 3BNC60, b12, F105, VRC01, VRC07, VRC07-523, VRC03, VRC06, VRC06b01 VRC08, VRC0801, NIH45-46, VRC-PG04, PGV04; CH103, 44-VRC13.01, 1NC9, 12A12, N6, N49-P7, NC-Cow1, IOMA, CH235 and CH235.12, N49P6, N49P7, N49P11, N49P9 and N60P25. In some embodiments, the method further comprises administering to the subject one or more anti-HIV broadly neutralizing antibodies that bind to an epitope or region of gp120 in the gp120/gp41 interface and competes with or comprises VH and VL regions from an antibody selected from the group consisting of PGT-151, CAP248-2B, 35022, 8ANC195, ACS202, VRC34 and VRC34.01. In some embodiments, the method further comprises administering to the subject one or more anti-HIV broadly neutralizing antibodies that bind to an epitope or region of gp120 silent face and competes with or comprises VH and VL regions from an antibody selected from VRC-PG05 and SF12. In some embodiments, the method further comprises administering to the subject one or more anti-HIV broadly neutralizing antibodies that bind to an epitope or region of gp41 in the membrane proximal region (MPER). In some embodiments, the one or more anti-HIV broadly neutralizing antibodies bind to an epitope or region of gp41 in the membrane proximal region (MPER) and competes with or comprises VH and VL regions from an antibody selected from the group consisting of 10E8, 10E8v4, 10E8-5R-100cF, 4E10, DH511.11P, 2F5, A32, 7b2, and LN01. In some embodiments, the one or more anti-HIV broadly neutralizing antibodies bind to an epitope or region of the gp41 fusion peptide and competes with or comprises VH and VL regions from an antibody selected from the group consisting of VRC34 and ACS202. In some embodiments, the subject is not receiving antiretroviral therapy (ART) or ART is discontinued prior to administration of the CD4-targeted IL-15, the polynucleotide or polynucleotides, the expression cassette, the vector, the LNP or the pharmaceutical composition. In some embodiments, ART is discontinued after one or more administrations of the CD4-targeted IL-15, the polynucleotide or polynucleotides, the expression cassette, the vector, the LNP or the pharmaceutical composition. In some embodiments, the method further comprises administering one or more antiretroviral therapy (ART) agents to the subject. In some embodiments, the method further comprises administering one or more innate immune activators. In some embodiments, the one or more innate immune activators comprises an agonist of a receptor selected from the group consisting of fms related tyrosine kinase 3 (FLT3), stimulator of interferon genes (STING) receptor, DExD/H-box helicase 58 (DDX58; a.k.a., RIG-I), NLR family pyrin domain containing 3 (NLRP3) and nucleotide binding oligomerization domain containing 2 (NOD2). In some embodiments, the method further comprises administering one or both of GS-3583 and inarigivir soproxil (GS-9992). In some embodiments, the method further comprises co-administering one or more antagonists or inhibitors of an inhibitory immune checkpoint protein or receptor and/or one or more activators or agonists of a stimulatory immune checkpoint protein or receptor. In some embodiments, the method further comprises the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160 (NK1, NK28, BY55), MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); sialic acid binding Ig like lectin 7 (SIGLEC7); sialic acid binding Ig like lectin 9 (SIGLEC9); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAET1L; ULBP6); killer cell immunoglobulin like receptor, three Ig domains and long cytoplasmic tail 1 (KIR, CD158E1); CD160; killer cell lectin like receptor B1 (KLRB1, CD161); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments, the method further comprises co-administering one or more blockers or inhibitors of one or more T-cell inhibitory immune checkpoint proteins or receptors. In some embodiments, the T-cell inhibitory immune checkpoint proteins or receptors are selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the method further comprises co-administering one or more agonists or activators of one or more T-cell stimulatory immune checkpoint proteins or receptors. In some embodiments, the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the method further comprises co-administering one or more blockers or inhibitors of one or more NK-cell inhibitory immune checkpoint proteins or receptors. In some embodiments, the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); CD160; killer cell lectin like receptor B1 (KLRB1, CD161); killer cell lectin like receptor C1 (KLRC1, NKG2A, CD159A); killer cell lectin like receptor D1 (KLRD1, CD94), killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments, the method further comprises co-administering one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the one or more immune checkpoint inhibitors comprises a proteinaceous (e.g., antibody) inhibitor of PD-L1 (CD274), PD-1 (PDCD1) or CTLA4. In some embodiments, the proteinaceous (e.g., antibody) inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous (e.g., antibody) inhibitor of PD-L1 (CD274) or PD-1 (PDCD1) is selected from the group consisting of zimberelimab (AB122), pembrolizumab, nivolumab, cemiplimab, pidilizumab, AMP-224, MEDI0680 (AMP-514), spartalizumab, atezolizumab, avelumab, ASC22, durvalumab, BMS-936559, CK-301, envafolimab (ASC-22, KN-035), PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, budigalimab (ABBV-181), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments, the one or more immune checkpoint inhibitors comprises a small molecule inhibitor of CD274 (PDL1, PD-L1), programmed cell death 1 (PDCD1, PD1, PD-1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002. In some embodiments, the subject is chronically infected with HIV. In some embodiments, the subject is heavily treatment experienced (HTE). In some embodiments the CD4-targeted IL-15, the polynucleotide, the vector, the LNP and/or the pharmaceutical composition are administered systemically or locally. In some embodiments, the CD4-targeted IL-15, the polynucleotide, the vector, the LNP and/or the pharmaceutical composition is administered via a route selected from intravenous, subcutaneous, intramuscular, intradermal, and mucosal (e.g. buccal, intranasal, intrarectal, intravaginal). In some embodiments, the CD4-targeted IL-15, the polynucleotide, the vector, the LNP and/or the pharmaceutical composition and the one or more additional therapeutic agents are administered by the same routes of administration. In some embodiments, the CD4-targeted IL-15, the polynucleotide, the vector, the LNP and/or the pharmaceutical composition and the one or more additional therapeutic agents are administered by different routes of administration. In some embodiments, the CD4-targeted IL-15, the polynucleotide, the vector, the LNP and/or the pharmaceutical composition and the one or more additional therapeutic agents are co-administered according to the same schedule (e.g., co-administered at the same time intervals). In some embodiments, the CD4-targeted IL-15, the polynucleotide, the vector, the LNP and/or the pharmaceutical composition and the one or more additional therapeutic agents are co-administered according to different schedules (e.g., co-administered at different time intervals). In some embodiments, the method comprises multiple administrations of the CD4-targeted IL-15, the polynucleotide, the vector, the LNP and/or the pharmaceutical composition, optionally with one or more additional therapeutic agents, at predetermined intervals. In some embodiments, the IL-15v, IL-15Rα Sushi domain-IL-15v fusion protein or CD4-targeted IL-15v have a serum half-life in a human of less than 3 days, less than 2 days or less than 1 day. In some embodiments, the subject or the mammal is a human. In some embodiments, the IL-15v, IL-15Rα Sushi domain-IL-15v fusion protein or CD4-targeted IL-15v have a serum half-life in a human of less than 24 hours, e.g., less than 20 hours, less than 18 hours, less than 16 hours. In some embodiments, the subject or the mammal is a human.

Further provided is a kit comprising one or more unitary doses of an IL-15v, IL-15Rα Sushi domain-IL-15v fusion protein or CD4-targeted IL-15v, polynucleotide or polynucleotides encoding the IL-15v, IL-15Rα Sushi domain-IL-15v fusion protein or CD4-targeted IL-15v, an expression cassette of claim, a vector, a lipoplex (e.g, LNP) or a pharmaceutical composition comprising an IL-15v, IL-15Rα Sushi domain-IL-15v fusion protein or CD4-targeted IL-15v, as described above and herein. In some embodiments, the one or more unitary doses are in a single container. In some embodiments, the one or more unitary doses are in two or more separate containers. In some embodiments, the kit comprises one or more containers selected from the group consisting of vials, ampoules and pre-loaded syringes. In some embodiments, the one or more containers comprising the IL-15v, IL-15Rα Sushi domain-IL-15v fusion protein or CD4-targeted IL-15v, in an aqueous solution. In some embodiments, the one or more unitary doses are the same. In some embodiments, the kit comprises two or more unitary doses, wherein the unitary doses are the same. In some embodiments, the one or more unitary doses are different. In some embodiments, the kit comprises two or more unitary doses, wherein the unitary doses are different. In some embodiments, the kit further comprises one or more unitary doses of one or more additional therapeutic agents. In some embodiments, the kit further comprises at least one latency reversal agent (LRA). In some embodiments, the kit further comprises at least one toll-like receptor (TLR) agonist. In some embodiments, the TLR agonist is a TLR2 agonist, a TLR3 agonist, a TLR7 agonist, a TLR8 agonist or a TLR9 agonist. In some embodiments, the TLR3 agonist is selected from the group consisting of rintatolimod and Polyinosinic-Polycytidylic Acid Stabilized with Polylysine and Carboxymethylcellulose (poly ICLC (HILTONOL®)). In some embodiments, the TLR7 agonist is selected from the group consisting of vesatolimod, imiquimod, resiquimod and NKTR-262. In some embodiments, the TLR8 agonist is selected from the group consisting of selgantolimod, motolimod resiquimod and NKTR-262. In some embodiments, the TLR9 agonist is selected from the group consisting of cavrotolimod, cobitolimod, agatolimod, leftolimod, litenimod and tilsotolimod. In some embodiments, the kit further comprises a multispecific T-cell engager, e.g., a bispecific T-cell engager. In some embodiments, the kit further comprises a CD4-Fc fusion protein. In some embodiments, the kit further comprises one or more unitary doses of GS 8588. In some embodiments, the kit further comprises a lipid nanoparticle (LNP) comprising an HIV tat mRNA. In some embodiments, the kit further comprises a non-nucleoside reverse transcriptase inhibitor (NNRTI) as targeted activator of cell kill (TACK). In some embodiments, the TACK is selected from efavirenz (EFV), rilpivirine (RPV), pyrimidone pyr01, and pyrimidone pyr02. In some embodiments, the kit further comprises a Second Mitochondria-derived Activator of Caspase (SMAC) mimetic or an inhibitor of cellular inhibitor of apoptosis proteins (cIAP). In some embodiments, the SMAC mimetic or cIAP inhibitor is selected from xevinapant, dasminapant, birinapant, tolinapant lactate, ciapavir and AZD5582. In some embodiments, the kit further comprises an inhibitor of one or both of protein tyrosine phosphatase non-receptor type 1 (PTPN1) and protein tyrosine phosphatase non-receptor type 2 (PTPN2). In some embodiments, the inhibitor of one or both of PTPN1 and PTPN2 is selected from 3-Hydroxy-1,2,3-benzotriazin-4(3H)-one (HODHBt), avarol, osunprotafib, trodusquemine, S nitrosoglutathione, S-nitroso-N-acetylpenicillamine, talaramide Q, aurothiomalate disodium, daiokanzoto, berberine chloride, and alpha-tocopherolquinone. In some embodiments, the kit further comprises one or more anti-HIV vaccines. In some embodiments, the anti-HIV vaccine is a viral vector vaccine. In some embodiments, the viral vector is selected from an arenavirus vector, modified vaccinia virus Ankara (MVA)) vector, and a simian (e.g., chimpanzee, gorilla, rhesus) adenovirus vector. In some embodiments, the kit further comprises one or more anti-HIV broadly neutralizing antibodies that bind to different epitopes or regions of gp120 selected from the group consisting of: (i) third variable loop (V3) (e.g., high mannose patch) comprising a N332 oligomannose glycan; (ii) second variable loop (V2) (e.g., Env trimer apex); (iii) CD4 binding site (CD4bs); (iv) gp120/gp41 interface; or (v) silent face of gp120. In some embodiments, the kit further comprises an anti-HIV broadly neutralizing antibody that binds to the third variable loop (V3) (e.g., high mannose patch) comprising a N332 oligomannose glycan and the second antigen binding molecule binds to the CD4 binding site (CD4bs). In some embodiments, the kit further comprises an anti-HIV broadly neutralizing antibody that competes with or comprises VH and VL regions from an antibody selected from the group consisting of zinlirvimab (GS-2872), elipovimab (GS-9722), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the kit further comprises an anti-HIV broadly neutralizing antibody that competes with or comprises VH and VL regions from an antibody selected from the group consisting of zinlirvimab (GS-2872), 10-1074, 10-1074-J, elipovimab (GS-9722), PGT-121, PGT-121.66, PGT-121.414, PGT-128 and PGT-134. In some embodiments, the kit further comprises an anti-HIV broadly neutralizing antibody that competes with or comprises VH and VL regions from an antibody selected from the group consisting of teropavimab (GS-5423), GS-9723, 3BNC117, 3BNC60, b12, F105, VRC01, VRC07, VRC07-523, VRC03, VRC06, VRC06b01 VRC08, VRC0801, NIH45-46, PGV04 (VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25. In some embodiments, the kit further comprises an anti-HIV broadly neutralizing antibody that competes with or comprises VH and VL regions from an antibody selected from the group consisting of teropavimab (GS-5423), GS-9723, 3BNC117, VRC07 and VRC07-523. In some embodiments, the kit further comprises an anti-HIV broadly neutralizing antibody that competes with or comprises VH and VL regions from an antibody that binds to the gp120 second variable loop (V2) (e.g., Env trimer apex). In some embodiments, the kit further comprises an anti-HIV broadly neutralizing antibody that competes with or comprises VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the kit further comprises an anti-HIV broadly neutralizing antibody that competes with or comprises VH and VL regions from an antibody that binds to the gp120/gp41 interface. In some embodiments, the kit further comprises an anti-HIV broadly neutralizing antibody that competes with or comprises VH and VL regions from an antibody selected from the group consisting of PGT-151, CAP248-2B, 35022, 8ANC195, ACS202, VRC34 and VRC34.01. In some embodiments, the kit further comprises an anti-HIV broadly neutralizing antibody that competes with or comprises VH and VL regions from an antibody that binds to an epitope or region of gp41 in the membrane proximal region (MPER). In some embodiments, the kit further comprises an anti-HIV broadly neutralizing antibody that competes with or comprises VH and VL regions from an antibody selected from the group consisting of 10E8, 10E8v4, 10E8-5R-100cF, 4E10, DH511.11P, 2F5, A32, 7b2, and LN01. In some embodiments, the kit further comprises one or more innate immune activators. In some embodiments, the kit further comprises an agonist of a receptor selected from the group consisting of fms related tyrosine kinase 3 (FLT3), stimulator of interferon genes (STING) receptor, DExD/H-box helicase 58 (DDX58; a.k.a., RIG-I), NLR family pyrin domain containing 3 (NLRP3) and nucleotide binding oligomerization domain containing 2 (NOD2). In some embodiments, the kit further comprises one or both of GS-3583 and inarigivir soproxil (GS-9992). In some embodiments, the kit further comprises one or more antagonists or inhibitors of an inhibitory immune checkpoint protein or receptor and/or one or more activators or agonists of a stimulatory immune checkpoint protein or receptor. In some embodiments, the one or more immune checkpoint proteins or receptors are selected from the group consisting of: CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160 (NK1, NK28, BY55), MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CD112R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); sialic acid binding Ig like lectin 7 (SIGLEC7); sialic acid binding Ig like lectin 9 (SIGLEC9); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAET1L; ULBP6); killer cell immunoglobulin like receptor, three Ig domains and long cytoplasmic tail 1 (KIR, CD158E1); CD160; killer cell lectin like receptor B1 (KLRB1, CD161); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments, the kit further comprises one or more blockers or inhibitors of one or more T-cell inhibitory immune checkpoint proteins or receptors. In some embodiments, the T-cell inhibitory immune checkpoint proteins or receptors are selected from the group consisting of CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In some embodiments, the kit further comprises one or more agonists or activators of one or more T-cell stimulatory immune checkpoint proteins or receptors. In some embodiments the T-cell stimulatory immune checkpoint proteins or receptors are selected from the group consisting of CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); Poliovirus receptor (PVR) cell adhesion molecule (PVR, CD155). In some embodiments, the kit further comprises one or more blockers or inhibitors of one or more NK-cell inhibitory immune checkpoint proteins or receptors. In some embodiments, the NK-cell inhibitory immune checkpoint proteins or receptors are selected from the group consisting of killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); CD160; killer cell lectin like receptor B1 (KLRB1, CD161); killer cell lectin like receptor C1 (KLRC1, NKG2A, CD159A); killer cell lectin like receptor D1 (KLRD1, CD94); killer cell lectin like receptor G1 (KLRG1; CLEC15A, MAFA, 2F1); sialic acid binding Ig like lectin 7 (SIGLEC7); and sialic acid binding Ig like lectin 9 (SIGLEC9). In some embodiments, the kit further comprises one or more agonists or activators of one or more NK-cell stimulatory immune checkpoint proteins or receptors. In some embodiments, the NK-cell stimulatory immune checkpoint proteins or receptors are selected from CD16, CD226 (DNAM-1); killer cell lectin like receptor K1 (KLRK1, NKG2D, CD314); and SLAM family member 7 (SLAMF7). In some embodiments, the kit further comprises a proteinaceous (e.g., antibody) inhibitor of PD-L1 (CD274), PD-1 (PDCD1) or CTLA4. In some embodiments, the proteinaceous (e.g., antibody) inhibitor of CTLA4 is selected from the group consisting of ipilimumab, tremelimumab, BMS-986218, AGEN1181, AGEN1884 (zalifrelimab), BMS-986249, MK-1308, REGN-4659, ADU-1604, CS-1002, BCD-145, APL-509, JS-007, BA-3071, ONC-392, AGEN2041, JHL-1155, KN-044, CG-0161, ATOR-1144, PBI-5D3H5, FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4) and AK-104 (CTLA4/PD-1). In some embodiments, the proteinaceous (e.g., antibody) inhibitor of PD-L1 (CD274) or PD-1 (PDCD1) is selected from the group consisting of zimberelimab (AB122), pembrolizumab, nivolumab, cemiplimab, pidilizumab, AMP-224, MEDI0680 (AMP-514), spartalizumab, atezolizumab, avelumab, ASC22, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN2034 (balstilimab), JS-001 (toripalimab), JNJ-63723283, genolimzumab (CBT-501), LZM-009, BCD-100, LY-3300054, SHR-1201, SHR-1210 (camrelizumab), Sym-021, ABBV-181, PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1). In some embodiments, the kit further comprises a small molecule inhibitor of CD274 (PDL1, PD-L1), programmed cell death 1 (PDCD1, PD1, PD-1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

### BRIEF DESCRIPTION OF THE DRAWINGS

FIGS. 1A-1F illustrate the design of (A) IL-15-Fc/IL-15Rα-Fc fusion molecules, (B) N-terminal IL-15-Fc fusion CD4-targeted molecules, (C and D) N-terminal IL-15Rα-IL-15-Fc fusion CD4-targeted molecules, (E) C-terminal Fc-IL-15-IL-15Rα CD4-targeted molecules and (F) C-terminal Fc-IL-15Rα-IL-15 CD4-targeted IL-15 molecules.

FIGS. 2A-2D illustrate in vitro potency of Fc-IL-15 variant fusion proteins on CD4+ T cell, CD8+ T cell and NK cell proliferation.

FIG. 3 illustrates cryoelectron microscopy (Cryo-EM) structure of human anti-CD4 Fab 1.22 bound to human CD4 at 2.7 Å. The determined atomic structure confirms that the CD4 targeting arm binds to domain 3 of human CD4 and that the SNP positions Phe227 and Arg265 do not participate in the binding.

FIG. 4 illustrates epitope of human CD4 recognized by the CD4 targeting antigen binding domain 1.22. The atomic structure of the complex between human CD4 and the anti-CD4 Fab 1.22 shows that the recognized residues are exclusively in domain 3 (D3) of CD4. The CD4 epitope is formed by residues 218, 220, 260, 271, 274-277, 279, 283 and 285. A CD4 residue was considered to be in contact with the Fab if the van-der-Waals radius of any of its atoms was within 0.4 Å of an atom in the Fab as implemented in the analysis and visualization software program Chimera (rbvi.ucsf.edu/chimera/).

FIG. 5 illustrates binding to human CD4 (Arg265, dark grey bars) or human CD4 with a common polymorphism (hCD4.Trp265, light grey bars), as determined by ELISA. X-axis refers to anti-CD4 antibody clones; summarized in Tables A1-A4 and B. Y axis refers to optical density (OD) at 450 nm.

FIGS. 6A-6H illustrate graphics showing the IL-15 receptor quaternary complex (FIG. 6A), IL-15 interface with contact amino acid residues selected for substitution, e.g., S7, D61, E65, N65, 168 and Q108 (FIG. 6B) and interaction of these contact residues with surrounding residues from IL-2Rβγ (complex of CD122 (NCBI Gene ID: 3560) and CD132 (NCBI Gene ID: 3561)) (FIGS. 6C-6H). MLY refers to methylated lysine 71.

FIG. 7 illustrates predicted stability of possible variants designed to mitigate glycan and deamidation liabilities.

FIG. 8 illustrates the IL-15 receptor quaternary complex in stick representation with IL-15 N-linked glycosylation and deamidation sites labeled.

FIGS. 9A-9B illustrate HIV virion RNA production after treatment with DMSO (vehicle control), 1 nM recombinant human IL-15, 1 nM CD4-targeted IL-15 (Molecule 101), or PMA and ionomycin in peripheral blood mononuclear cells (PBMCs) from anti-retroviral therapy (ART)-suppressed people with HIV (PWH) (Panel 9A; N=17). Each symbol represents the geometric mean of six replicates from a single donor. Panel 9B shows paired comparison of IL-15 and CD4-targeted IL-15 (Molecule 101). Lines connect symbols representing results from the same donor. Significance was assessed by Wilcoxon matched pairs signed rank test.

FIG. 10 illustrates HIV virion RNA production after treatment with CD4-targeted IL-15 in PBMCs from ART-suppressed PWH and activation of Ki67 expression. HIV viral reactivation ex vivo is plotted using the left axis and shown with dashed line and closed symbols. Ki67 expression is plotted on the right axis and shown with solid line and open symbols.

FIG. 11 illustrates HIV virion RNA production after treatment with multiple CD4-targeted IL-15 molecules in PBMCs from ART-suppressed PWH. Each symbol represents the geometric mean of six replicates from a single donor.

FIG. 12 illustrates HIV virion RNA production after treatment with pattern recognition receptor agonists in PBMCs from ART-suppressed PWH. Each symbol represents the geometric mean of twelve replicates from a single donor. Conditions significantly different from the vehicle control were determined by Wilcoxon matched pairs signed rank test.

FIGS. 13A-13B. FIG. 13A illustrates HIV virion RNA production after treatment with TLR7, TLR8, NOD2, TLR3, TLR2 agonists with or without recombinant human IL-15 in PBMCs from ART-suppressed PWH (N=7). “P/I” indicates the positive control condition, treatment with PMA and ionomycin. Each symbol represents the geometric mean of 12 replicates from a single donor. Significance was assessed by Wilcoxon matched pairs signed rank test. FIG. 13B illustrates combined effects (e.g., synergy) calculated using Bliss independence model for TLR7, TLR8, NOD2, TLR3, TLR2 agonists with combination with recombinant human IL-15.

FIGS. 14A-14F. FIGS. 14A-14C illustrate HIV virion RNA production after treatment with TLR8 (14A), TLR2 (14B) or NOD2 (14C) agonists with or without recombinant human IL-15 in PBMCs from ART-suppressed PWH (N=16). “PMA+Iono” indicates the positive control condition, treatment with PMA and ionomycin. Each symbol represents the geometric mean of 12 replicates from a single donor. Significance was assessed by Wilcoxon matched pairs signed rank test. FIGS. 14D-14F demonstrate synergy for HIV virion RNA production after treatment of recombinant human IL-15 combined with TLR8 (14D), TLR2 (14E) or NOD2 (14F) agonists. Combined effects (e.g., synergy) were calculated using Bliss independence model and significance was assessed by paired two tailed t-test.

FIGS. 15A-15C. FIG. 15A illustrates HIV virion RNA production after treatment with TLR8 or NOD2 agonists with or without CD4-targeted IL-15v (Molecule 277; Table 20) in PBMCs from ART-suppressed PWH (N=16). Each symbol represents the geometric mean of 12 replicates from a single donor. Significance was assessed by Wilcoxon matched pairs signed rank test. FIGS. 15B and 15C demonstrate synergy observed after treatment of TLR8 (15B) and NOD2 (15C) with CD4-targeted IL-15v. Combined effects (e.g., synergy) were calculated using Bliss independence model and significance was assessed by paired two tailed t-test.

FIGS. 16A-16D. FIGS. 16A-B provide structures of illustrative HIV protease activators (a.k.a., non-nucleoside reverse transcriptase inhibitors (NNRTIs) as targeted activators of cell kill (TACK)) used in combination assays. FIG. 16A provides structure of HIV protease activator used in combination with Molecule 101. FIG. 16B provides structure of HIV protease activator used in combination with Molecule 173. FIGS. 16C-D illustrate virion production after treatment of PBMCs from ART-suppressed PWH with CD4-targeted IL-15 with (FIG. 16C) bNAbs (N=8 for Molecule 101; N=7 for Molecule 173) or (FIG. 16D) HIV protease activator (N=17 for Molecule 101 and Molecule 173). Each symbol represents the geometric mean of eight replicates from a single donor. Lines connect samples from the same donor. Geometric means for each condition are shown at the top of the graph. Significance was assessed by Wilcoxon matched pairs signed rank test.

FIGS. 17A-17B. FIG. 17A illustrates killing of HIV-infected CEM-NKr-CCR5-LucR+ cells by a dose titration of amtabafusp alfa (GS-8588) with a high concentration of each indicated CD4-targeted IL-15v molecule. FIG. 17B illustrates the percent inhibition of amtabafusp alfa (GS-8588)-mediated killing of HIV-infected CEM-NKr-CCR5-LucR+ cells when pre-incubated with a dose titration of each indicated CD4-targeted IL-15v.

FIGS. 18A-181 illustrate single dose pharmacokinetic curves for (FIG. 18A) Molecule 263 in rhesus macaques, (FIG. 18B) Molecule 264 in rhesus macaques, (FIG. 18C) Molecule 247 in rhesus macaques, (FIG. 18D) Molecule 247 in cynomolgus macaques, (FIG. 18E) Molecule 100 in cynomolgus macaques, (FIG. 18F) Molecule 101 in cynomolgus macaques, (FIG. 18G) Molecule 109 in cynomolgus macaques, (FIG. 18H) Molecule 173 in cynomolgus macaques, and (FIG. 18I) Molecule 175 in cynomolgus macaques.

FIGS. 19A-19I illustrate the effect of single dose administration on CD4+T cells, CD8+ T cells and NK cells after dosing with (FIG. 19A) Molecule 263 in rhesus macaques, (FIG. 19B) Molecule 264 in rhesus macaques, (FIG. 19C) Molecule 247 in rhesus macaques, (FIG. 19D) Molecule 247 in cynomolgus macaques, (FIG. 19E) Molecule 100 in cynomolgus macaques, (FIG. 19F) Molecule 101 in cynomolgus macaques, (FIG. 19G) Molecule 109 in cynomolgus macaques, (FIG. 19H) Molecule 173 in cynomolgus macaques, and (FIG. 19I) Molecule 175 in cynomolgus macaques.

FIG. 20 illustrates a study design with a rhesus CD4-targeted IL-15 tool molecule, Molecule 247, in SIVmac251-infected, ART-suppressed rhesus macaques. Rhesus macaques were infected for over a year and then treated with daily with subcutaneous antiretroviral therapy (ART: 2.5 mg/ml dolutegravir (DTG), 40 mg/ml emtricitabine (FTC), 5.1 mg/ml tenofovir disoproxil fumarate (TDF)) for a year prior to therapeutic intervention. Animals were administered either Molecule 247 or vehicle control (placebo) intravenously (IV), biweekly for a total of six doses in the presence of ART as shown.

FIG. 21 illustrates SIV viral reactivation activity by Molecule 247 in chronically infected, ART suppressed rhesus macaques. Plasma SIV viral RNA was assessed by SIV Gag RNA-specific quantitative reverse transcriptase polymerase chain reaction (qRT-PCR) during dosing and up to 6 months after the last dose. PCR assays with 8 and 15 copy/ml lower limits of quantification (LLOQs) were utilized: 15 copy/ml assay was used from day 47 to 51 and day 85 to 238 and the 8 copy/ml assay was used for all other timepoints. Viral loads for individual animals in the vehicle (intact lines) and CD4-targeted IL15v (broken lines) groups are shown.

FIG. 22 illustrates CD4+ T cell counts measured after the first, fourth and sixth dose of Molecule 247 or the vehicle control from the study shown in FIG. 20.

FIGS. 23A-23B illustrate the activation of Ki67 in naïve and memory CD4+T cells after the first, fourth and sixth doses of Molecule 247 (FIG. 23A) or the vehicle control (FIG. 23B) from the study shown in FIG. 20.

FIG. 24 illustrates the study design for Molecule 247 dosed in combination with bNAb, PGT-121, in chronically infected, ART suppressed rhesus macaques. SHIV-SF162P3 infected and ART (DTG+FTC+TDF) suppressed rhesus macaques were treated as shown. Upon completion of dosing and PGT-121 washout, animals were released from ART and followed for up to 9 months to study viral rebound kinetics in plasma.

FIG. 25 illustrates viral rebound kinetics following ART treatment interruption in the 3 groups shown in FIG. 24.

FIG. 26 illustrates a study design for a combination study of CD4-targeted IL-15 (Molecule 247) with AZD5582 in ART-suppressed SIV-infected rhesus macaques.

FIGS. 27A-27C illustrate SIV viral reactivation in the study shown in FIG. 26. Plasma viral RNA was assessed by SIV Gag RNA-specific quantitative reverse transcriptase polymerase chain reaction (qRT-PCR) during dosing. PCR assay with 3 copy/ml lower limits of quantification (LLOQs) were utilized. Viral loads for individual animals from all three experimental groups were shown.

FIGS. 28A-28C illustrate IL-15v-mediated enhancement of tumor cell killing in vitro. NK cell proliferation (FIG. 28A), CD107a-mediated NK cell degranulation (FIG. 28B) and cytotoxicity (FIG. 28C) of Raji cells upon treatment with recombinant human IL-15 (rhIL-15) or IL-15 mutein with high affinity (N65D; IL-15m (Hi)) or medium affinity (S7G_N65D; IL-15m (Med)) or low affinity (N65D_168A; IL-15m (Low)) or isotype control. Cells were incubated 150 nM of indicated reagent and 1 nM of CD20-targeted NKG2D NK cell engager. Cytotoxicity data in (FIG. 28C) is normalized to Raji cell in control conditions.

### DETAILED DESCRIPTION

### 1. Introduction

Provided are multi-specific or bispecific molecules, targeting CD4 and IL-15 receptor (CD122/CD132), designed for high selectivity, improved tolerability, drug-like properties, and manufacturing efficiency. The herein described CD4-targeted IL-15v molecules include three components: (1) a CD4 binding domain, (2) a fusion protein comprising an IL-15 receptor alpha (IL-15Rα) Sushi domain and an IL-15 variant, and (3) a Fc domain.

The CD4 binding arm (i.e., antigen binding domain) is derived from a human sequence antibody sequence and selected for high affinity to CD4, low polyspecificity, high thermodynamic stability, low sequence liabilities for manufacturing and low immunogenicity. In some embodiments, the CD4 binding domain is specific for CD4 domain 3 (D3), avoiding competition with the natural CD4 ligand, MHC II, or with HIV Env, both of which bind CD4 domain 1 (D1). In some embodiments, a CD4 binding domain binds to CD4 in the presence or absence of commonly known polymorphisms, e.g., CD4 polymorphism variant IDs rs28919570 (R265W), rs11064419 (F227S or F227C) and rs11064416 (F123L).

The fusion protein has an IL-15Rα Sushi domain, a flexible linker and a variant of human IL-15. This IL-15Rα Sushi domain-IL-15v fusion design mimics the natural interaction between IL-15 and IL-15Rα, which typically binds IL-15 as the cytokine is synthesized and facilitates trans presentation to IL-15 receptor beta/gamma (IL-15Rβγ; CD122/CD132). Because IL-15Rα facilitates production and secretion of IL-15 under physiological conditions, acting as a chaperone that improves IL-15 production, the IL-15Rα Sushi domain-IL-15v fusion protein improves production of the IL-15v-based molecule. See, e.g., Bergamaschi, et al., J Immunol (2009) 183 (5):3064-72. In some embodiments, amino acid substitutions have been introduced into the human IL-15 that decrease its affinity for the IL-15Rβγ (CD122/CD132). By reducing the affinity of the IL-15Rα Sushi domain-IL-15v fusion protein for its receptor, these amino acid substitutions significantly increase the pharmacokinetic properties of the molecule, mitigating the target mediated drug disposition that typically limits the stability of IL-15 molecules (Lu et al., Eur J Pharm Sci (2023) 186:106450).

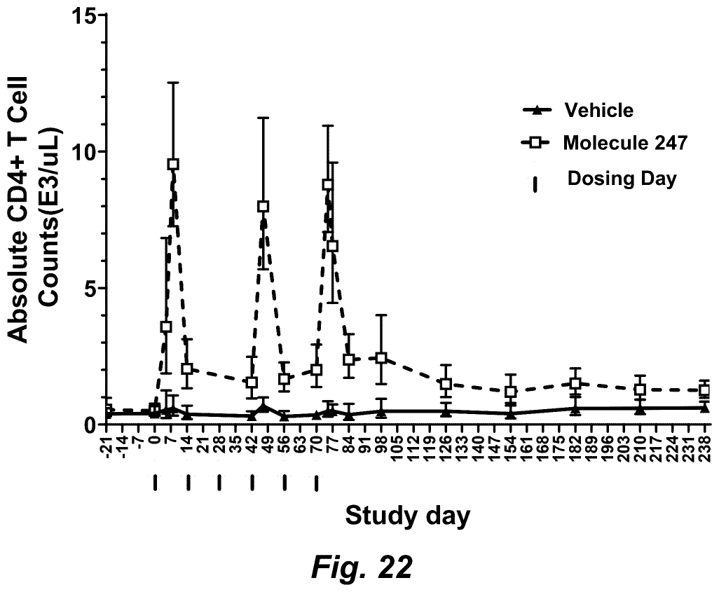

In certain embodiments, the CD4-targeted IL-15v molecules are designed to have a binding affinity for CD4 that is at least about 1000-fold stronger than a binding affinity for IL-15Rβγ. The attenuation of IL-15Rβγ binding reduces the activation of cells that do not express the CD4 receptor, including NK and CD8+ T cells, which are typically strongly activated by wild-type IL-15. The herein described CD4-targeted IL-15v molecules are designed to achieve attenuation with a minimal combination of amino acids substitutions to the wild-type IL-15 sufficient to reduce binding affinity to the IL-15Rβγ or IL-2Rβγ complex (CD122/CD132) and to reduce or minimize the potential for immunogenicity (e.g., anti-drug antibodies). The present disclosure demonstrates that the herein described CD4-targeted IL-15 molecules achieve higher levels of in vivo CD4+ T cell stimulation than have previously been reported with untargeted IL-15 molecules and that this activation surprisingly includes both naïve and memory CD4+ T cells. See, e.g., Example 14 and FIG. 22, which shows 10-fold expansion of CD4+ T cells. Additionally, the herein described CD4-targeted IL-15 molecules unexpectedly induce higher levels of HIV activation in comparison to wild-type IL-15, even when both molecules are used at saturating concentrations. Further, the monovalent IL-15Rα Sushi domain-IL-15v fusion protein in the format design decreases the likelihood of avidity-driven binding to IL-15 receptors.

The Fc domain of the herein described CD4-targeted IL-15 molecules is designed to facilitate desired heterodimerization of the two heavy chains. As used herein, a “heavy chain” refers to a full-length polypeptide and may or may not contain immunoglobulin domains. Manufacturability is enhanced by substitutions in the Fc domain that reduce Protein A binding on one Fc domain polypeptide, allowing the use of Protein A-based affinity chromatography to efficiently enrich for heterodimers over the corresponding homodimer contaminants. Additional substitutions were introduced to the Fc region to abrogate binding to Fc gamma receptor (FcγR) and reduce or eliminate the potential for antibody-dependent cell-mediated cytotoxicity (ADCC), antibody-dependent cellular phagocytosis (ADCP) or complement-dependent cytotoxicity (CDC). Furthermore, in the CD4-targeted IL-15 molecules described herein, it is possible that the C-terminal lysine of the heavy and light chains might be clipped. Accordingly, in various embodiments of the CD4-targeted IL-15 molecules described herein, the C-terminal lysine of the heavy and light chains is present or absent.

The herein described CD4-targeted IL-15v provide a therapeutic modality that reduces the latent reservoir and enhances HIV control in the absence of daily or regular antiretroviral therapy (Dybul et al., Lancet HIV (2021) 8:e51-58). Activation of HIV expression in latently infected cells increases the expression of HIV proteins and improves the efficacy of reservoir targeting modalities. The herein described CD4-targeted IL-15v improve cytokine targeting and tolerability to facilitate robust HIV activation and enhanced clearance of viral reservoirs, an important part of a strategy to induce HIV control in the absence of therapy.

### 2. CD4 Binding Domains

Provided are antibodies and antigen-binding fragments thereof that specifically bind to CD4, e.g., human CD4 (NCBI Gene ID: 920; Uniprot P01730). The extracellular (EC) portion of CD4 comprises four immunoglobulin-like domains (D1-D4), comprised of residues 1-369 (Wu, et al., Nature (1997) 387 (6632):527-30). In various embodiments, the CD4 binding domains specifically bind to one or more of domain 1 (D1), domain 2, (D2) or domain 3 (D3) of CD4, e.g., human CD4. The structure of human CD4 is described, e.g., in Wang, et al., Nature (1990) 348:411-418; Ryu, et al., Nature (1990) 348(6300):419-26; Leahy, FASEB J (1995) 9(1):17-25; Barclay, et al., Philos Trans R Soc Lond B Biol Sci (1993) 342(1299):7-12; Garrett, et al., J Mol Biol (1993) 234(3):763-78; Sakihama, et al., Immunology Today (1995) 16(12):581-87; and Wu, et al., Nature (1997) 387(6632):527-30. According to Ryu, et al., the first domain (D1) of CD4 comprises residues 1-98 and the second domain (D2) of CD4 contains residues 99-173. According to Wu, et al., residues 179-181 connect D1D2 to D3D4 and D3D4 is within residues 182-361, residues 362-363 being a carboxy-terminal extension, and residues 364-369 may provide a flexible linkage to the transmembrane segment. Freeman, et al., Structure (2010) 18(12):1632-41 describes the crystal structure of ibalizumab Fab fragment in complex with the first two domains (D1-D2) of CD4 at 2.2A° resolution. Matthias, et al., Nat Immunol (2002) 3(8):727-32 reports that the CD4 D1, D2 and D4 domains each contain a disulfide bod and that the D2 disulfide bond is redox-active. As used herein, the signal peptide of human CD4 corresponds to amino acid residues 1-25, CD4 EC D1 corresponds to amino acid residues 26-125, CD4 EC D2 corresponds to amino acid residues 126-203, CD4 EC D3 corresponds to amino acid residues 204-317, and CD4 EC D1 corresponds to amino acid residues 318-374, wherein the amino acid residue positions are with respect to SEQ ID NO: 1120 (see, e.g., uniprot.org/uniprotkb/P01730/entry).

MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI (human CD4; Uniprot P01730; SEQ ID NO:
1120).

As used herein, numbering of a given amino acid polymer or nucleic acid polymer “corresponds to”, is “corresponding to” or is “relative to” the numbering of a selected or reference amino acid polymer or nucleic acid polymer when the position of any given polymer component (e.g., amino acid, nucleotide, also referred to generically as a “residue”) is designated by reference to the same or to an equivalent position (e.g., based on an optimal alignment or a consensus sequence) in the selected amino acid or nucleic acid polymer, rather than by the actual numerical position of the component in the given polymer.

With regard to the binding of an antibody or antigen-binding fragment thereof to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. An antibody or antigen-binding fragment thereof that “specifically binds to” or is “specific for” a particular polypeptide or an epitope on a particular polypeptide is one that binds to that particular polypeptide or epitope on a particular polypeptide without substantially binding to any other polypeptide or polypeptide epitope. Affinities of antibodies can be readily determined using conventional techniques, for example, those described by Scatchard et al. (Ann. N. Y. Acad. Sci. USA 51:660 (1949), ELISA assays, biolayer interferometry (BLI) assays, and surface plasmon resonance (SPR) assays). Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

“Antigen-binding antibody fragments” comprise a portion of an intact antibody, for example, the antigen-binding or variable region of the intact antibody. Examples of antibody fragments include without limitation scFv, sc(Fv)2, Fab, F(ab)2, Fab′, F(ab′)2, Facb, and Fv fragments; diabodies; linear F (ab′)2 fragments (e.g., Zapata et al., Protein Eng. (1995) 8(10): 1057-1062); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F (ab′)2 fragment that has two antigen combining sites and is still capable of cross-linking antigen. Antigen-binding antibody fragments of use are reviewed in, e.g., Kitten, et al., Med Sci (Paris). (2019) 35(12):1092-1097 and Chiu, et al., Antibodies (Basel). (2019) 8(4):55.

Antigen Binding Domains that Specifically Bind to CD4 D1

Provided are antibodies or antigen-binding fragments thereof that specifically bind to CD4 D1.

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of:
1) SEQ ID NOs: 1, 2, 3, 4, 5 and 6; (1)
2) SEQ ID NOs: 1, 7, 3, 4, 8 and 9; (2)
3) SEQ ID NOs: 1, 10, 3, 11, 5 and 6; (3)
4) SEQ ID NOs: 1, 10, 3, 11, 12 and 6; (4)
5) SEQ ID NOs: 1, 10, 3, 11, 8 and 9; (5)
6) SEQ ID NOs: 1, 13, 3, 11, 8 and 9; (6)
7) SEQ ID NOs: 1, 14, 3, 11, 8 and 9; (7)
8) SEQ ID NOs: 1, 15, 3, 11, 8 and 9; (8)
9) SEQ ID NOs: 1, 16, 3, 11, 8 and 9; (9)
10) SEQ ID NOs: 1, 15, 3, 11, 12 and 17; (10)
11) SEQ ID NOs: 1, 16, 3, 11, 12 and 18; (11)
12) SEQ ID NOs: 1, 1078, 3, 11, 8 and 9; (12)
13) SEQ ID NOs: 1, 1079, 3, 11, 8 and 9; (13, 14, 17, 18 and 19)
14) SEQ ID NOs: 1, 1080, 3, 11, 8 and 9; (15)
15) SEQ ID NOs: 1, 1081, 3, 11, 8 and 9; (16)
16) SEQ ID NOs: 19, 20, 21, 22, 23 and 24; (1.17)
17) SEQ ID NOs: 25, 26, 27, 28, 29 and 30; (1.25)
18) SEQ ID NOs: 31, 32, 33, 34, 29 and 35; (1.26)
19) SEQ ID NOs: 25, 36, 37, 28, 29 and 38; (1.27)
20) SEQ ID NOs: 25, 1082, 39, 28, 29 and 38; (1.28)
21) SEQ ID NOs: 40, 41, 42, 28, 29 and 35; (1.29)
22) SEQ ID NOs: 25, 43, 39, 28, 29 and 38; (1.30)
23) SEQ ID NOs: 25, 44, 45, 28, 29 and 35; (1.31)
24) SEQ ID NOs: 25, 46, 47, 28, 29 and 35; (1.32)
25) SEQ ID NOs: 25, 48, 49, 28, 29 and 35; (1.33)
26) SEQ ID NOs: 25, 48, 50, 28, 29 and 35; (1.34)
27) SEQ ID NOs: 51, 52, 53, 54, 55 and 56; (1.45) or
28) SEQ ID NOs: 51, 57, 53, 58, 55 and 56; (1.46, 1.47, 1.48).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to IMGT) of:
1) SEQ ID NOs: 178, 179, 180, 181, 182 and 6; (1)
2) SEQ ID NOs: 178, 183, 180, 181, 182 and 9; (2)
3) SEQ ID NOs: 178, 184, 180, 181, 182 and 6; (4)
4) SEQ ID NOs: 178, 184, 180, 181, 182 and 9; (3, 5)
5) SEQ ID NOs: 178, 185, 180, 181, 182 and 9; (6, 7, 12)
6) SEQ ID NOs: 178, 186, 180, 181, 182 and 9; (8)
7) SEQ ID NOs: 178, 187, 180, 181, 182 and 9; (9)
8) SEQ ID NOs: 178, 186, 180, 181, 182 and 17; (10)
9) SEQ ID NOs: 178, 187, 180, 181, 182 and 18; (11)
10) SEQ ID NOs: 178, 188, 180, 181, 182 and 9; (13, 14, 17, 18, 19)
11) SEQ ID NOs: 178, 189, 180, 181, 182 and 9; (15)
12) SEQ ID NOs: 178, 190, 180, 181, 182 and 9; (16)
13) SEQ ID NOs: 191, 192, 193, 194, 195 and 24; (1.17)
14) SEQ ID NOs: 196, 197, 198, 199, 200 and 30; (1.25)
15) SEQ ID NOs: 201, 202, 203, 199, 200 and 35; (1.26)
16) SEQ ID NOs: 204, 205, 206, 199, 200 and 38; (1.27)
17) SEQ ID NOs: 196, 207, 208, 199, 200 and 38; (1.28)
18) SEQ ID NOs: 209, 210, 211, 199, 200 and 35; (1.29)
19) SEQ ID NOs: 196, 212, 208, 199, 200 and 38; (1.30)
20) SEQ ID NOs: 196, 213, 214, 199, 200 and 35; (1.31)
21) SEQ ID NOs: 196, 213, 215, 199, 200 and 35; (1.32)
22) SEQ ID NOs: 196, 216, 217, 199, 200 and 35; (1.33)
23) SEQ ID NOs: 196, 216, 218, 199, 200 and 35; (1.34)
24) SEQ ID NOs: 219, 220, 221, 222, 223 and 56; (1.45) or
25) SEQ ID NOs: 219, 224, 221, 225, 223 and 56; (1.46, 1.47, 1.48).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Chothia) of:
1) SEQ ID NOs: 323, 324, 325, 326, 182 and 327; (1)
2) SEQ ID NOs: 323, 328, 325, 326, 182 and 329; (2)
3) SEQ ID NOs: 323, 330, 325, 326, 182 and 327; (4)
4) SEQ ID NOs: 323, 330, 325, 326, 182 and 329; (5)
5) SEQ ID NOs: 323, 331, 325, 326, 182 and 329; (6, 7, 12)
6) SEQ ID NOs: 323, 332, 325, 326, 182 and 329; (8)
7) SEQ ID NOs: 323, 333, 325, 326, 182 and 329; (9)
8) SEQ ID NOs: 323, 332, 325, 326, 182 and 334; (10)
9) SEQ ID NOs: 323, 333, 325, 326, 182 and 335; (11)
10) SEQ ID NOs: 323, 336, 325, 326, 182 and 329; (13, 14, 17, 18 and 19)
11) SEQ ID NOs: 323, 337, 325, 326, 182 and 329; (15)
12) SEQ ID NOs: 323, 338, 325, 326, 182 and 329; (16)
13) SEQ ID NOs: 339, 340, 341, 342, 195 and 1131; (1.17)
14) SEQ ID NOs: 344, 345, 346, 347, 200 and 348; (1.25)
15) SEQ ID NOs: 349, 350, 351, 347, 200 and 352; (1.26)
16) SEQ ID NOs: 353, 354, 355, 347, 200 and 356; (1.27)
17) SEQ ID NOs: 344, 350, 357, 347, 200 and 356; (1.28)
18) SEQ ID NOs: 358, 350, 359, 347, 200 and 352; (1.29)
19) SEQ ID NOs: 343, 354, 357, 347, 200 and 356; (1.30)
20) SEQ ID NOs: 343, 360, 361, 347, 200 and 352; (1.31)
21) SEQ ID NOs: 343, 360, 362, 347, 200 and 352; (1.32)
22) SEQ ID NOs: 343, 363, 364, 347, 200 and 352; (1.33)
23) SEQ ID NOs: 343, 363, 365, 347, 200 and 352; (1.34)
24) SEQ ID NOs: 366, 367, 368, 1084, 223 and 369; (1.45) or
25) SEQ ID NOs: 366, 367, 368, 370, 223 and 369; (1.46, 1.47, 1.48).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Honegger) of:
1) SEQ ID NOs: 463, 464, 465, 466, 467 and 327; (1)
2) SEQ ID NOs: 463, 468, 465, 466, 469 and 329; (2)
3) SEQ ID NOs: 463, 470, 465, 466, 467 and 327; (3)
4) SEQ ID NOs: 463, 470, 465, 466, 469 and 327; (4)
5) SEQ ID NOs: 463, 470, 465, 466, 469 and 329; (5)
6) SEQ ID NOs: 463, 1132, 465, 466, 469 and 329; (6)
7) SEQ ID NOs: 463, 471, 465, 466, 469 and 329; (7)
8) SEQ ID NOs: 463, 472, 465, 466, 469 and 329; (8)
9) SEQ ID NOs: 463, 473, 465, 466, 469 and 329; (9)
10) SEQ ID NOs: 463, 472, 465, 466, 474 and 334; (10)
11) SEQ ID NOs: 463, 473, 465, 466, 474 and 335; (11)
12) SEQ ID NOs: 463, 475, 465, 466, 469 and 329; (12)
13) SEQ ID NOs: 463, 478, 465, 466, 469 and 329; (13, 14, 17, 18 and 19)
14) SEQ ID NOs: 463, 476, 465, 466, 469 and 329; (15)
15) SEQ ID NOs: 463, 477, 465, 466, 469 and 329; (16)
16) SEQ ID NOs: 479, 480, 481, 482, 483 and 1131; (1.17)
17) SEQ ID NOs: 484, 485, 486, 487, 488 and 348; (1.25)
18) SEQ ID NOs: 489, 490, 491, 487, 492 and 352; (1.26)
19) SEQ ID NOs: 493, 494, 495, 487, 492 and 356; (1.27)
20) SEQ ID NOs: 484, 496, 497, 487, 492 and 356; (1.28)
21) SEQ ID NOs: 498, 499, 500, 487, 492 and 352; (1.29)
22) SEQ ID NOs: 484, 501, 497, 487, 492 and 356; (1.30)
23) SEQ ID NOs: 484, 502, 503, 487, 492 and 352; (1.31)
24) SEQ ID NOs: 484, 504, 505, 487, 506 and 352; (1.32)
25) SEQ ID NOs: 484, 507, 508, 487, 492 and 352; (1.33)
26) SEQ ID NOs: 484, 507, 509, 487, 492 and 352; (1.34)
27) SEQ ID NOs: 510, 511, 512, 513, 514 and 369; (1.45) or
28) SEQ ID NOs: 510, 515, 512, 516, 514 and 369; (1.46, 1.47, 1.48).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 625 and 626; (1)
2) SEQ ID NOs: 627 and 628; (2)
3) SEQ ID NOs: 629 and 630; (3)
4) SEQ ID NOs: 629 and 631; (4)
5) SEQ ID NOs: 629 and 632; (5)
6) SEQ ID NOs: 633 and 632; (6)
7) SEQ ID NOs: 634 and 632; (7)
8) SEQ ID NOs: 635 and 632; (8)
9) SEQ ID NOs: 636 and 632; (9)
10) SEQ ID NOs: 635 and 637; (10)
11) SEQ ID NOs: 636 and 638; (11)
12) SEQ ID NOs: 639 and 632; (12)
13) SEQ ID NOs: 640 and 632; (13)
14) SEQ ID NOs: 641 and 632; (14)
15) SEQ ID NOs: 642 and 632; (15)
16) SEQ ID NOs: 643 and 632; (16)
17) SEQ ID NOs: 640 and 644; (17)
18) SEQ ID NOs: 640 and 645; (18)
19) SEQ ID NOs: 640 and 646; (19)
20) SEQ ID NOs: 647 and 648; (1.17)
21) SEQ ID NOs: 649 and 650; (1.25)
22) SEQ ID NOs: 651 and 652; (1.26)
23) SEQ ID NOs: 653 and 654; (1.27)
24) SEQ ID NOs: 655 and 656; (1.28)
25) SEQ ID NOs: 657 and 658; (1.29)
26) SEQ ID NOs: 659 and 660; (1.30)
27) SEQ ID NOs: 661 and 662; (1.31)
28) SEQ ID NOs: 663 and 664; (1.32)
29) SEQ ID NOs: 665 and 666; (1.33)
30) SEQ ID NOs: 667 and 666; (1.34)
31) SEQ ID NOs: 668 and 669; (1.45)
32) SEQ ID NOs: 670 and 671; (1.46)
33) SEQ ID NOs: 672 and 673; (1.47) or
34) SEQ ID NOs: 672 and 671; (1.48).

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least 20, 25, 30, 35, 40, 45, 50, 55, 60, 65 or more amino acids or nucleotides in length, and can be up to the full length of the reference polypeptide or polynucleotide sequence. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. Otherwise, standard parameters can be used. The sequence comparison algorithm then calculates the percent sequence identity for the test sequence(s) relative to the reference sequence, based on the designated program parameters.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least 20 contiguous positions, usually 30 to 75, 40 to 50, or the full length of a sequence, in which a sequence may be compared to a reference sequence of the same number of contiguous positions after the two sequences are optimally aligned.

Optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (e.g., GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity are the Basic Local Alignment Search Tool (BLAST), BLAST 2.0 and PSI-BLAST algorithms, which are described in Altschul, et al., J. Mol. Biol. (1990) 215: 403-410, Altschul, et al., Nucleic Acids Res. (1977) 25: 3389-3402, and Altschul, et al., Nucleic Acids Res. (1997) 25(17):3389-402, respectively. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTP program (for polypeptide sequences) or the BLASTN program (for polynucleotide sequences) uses as defaults a word length (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLASTP algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Residue positions which are not identical can differ by conservative amino acid substitutions. Conservative amino acid substitutions refer to the interchangeability of residues having similar side chains. For example, a group of amino acids having aliphatic side chains is glycine (Gly, G), alanine (Ala, A), valine (Val, V), leucine (Leu, L), and isoleucine (Ile, I); a group of amino acids having aliphatic-hydroxyl side chains is serine (Ser, S) and threonine (Thr, T); a group of amino acids having amide-containing side chains is asparagine (Asn, N) and glutamine (Gln, Q); a group of amino acids having aromatic side chains is phenylalanine (Phe, F), tyrosine (Tyr, Y), and tryptophan (Trp, W); a group of amino acids having basic side chains is lysine (Lys, K), arginine (Arg, R), and histidine (His, H); and a group of amino acids having sulfur-containing side chains is cysteine (Cys, C) and methionine (Met, M). Further, glutamic acid (Glu, E) and aspartic acid (Asp, D) are conservative amino acid substitutions.

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 2, 3, 4, 5 and 6 (according to Kabat);
2) SEQ ID NOs: 178, 179, 180, 181, 182 and 6 (according to IMGT);
3) SEQ ID NOs: 323, 324, 325, 326, 182 and 327 (according to Chothia); or
4) SEQ ID NOs: 463, 464, 465, 466, 467 and 327 (according to Honegger).
   In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 625 and 626 (antibody 1).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 7, 3, 4, 8 and 9 (according to Kabat);
2) SEQ ID NOs: 178, 183, 180, 181, 182 and 9 (according to IMGT);
3) SEQ ID NOs: 323, 328, 325, 326, 182 and 329 (according to Chothia); or
4) SEQ ID NOs: 463, 468, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 627 and 628 (antibody 2).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 5 and 6 (according to Kabat);
2) SEQ ID NOs: 178, 184, 180, 181, 182 and 9 (according to IMGT);
3) SEQ ID NOs: 323, 330, 325, 326, 182 and 327 (according to Chothia); or
4) SEQ ID NOs: 463, 470, 465, 466, 467 and 327 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 630 (antibody 3).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 12 and 6 (according to Kabat);
2) SEQ ID NOs: 178, 184, 180, 181, 182 and 6 (according to IMGT);
3) SEQ ID NOs: 323, 330, 325, 326, 182 and 327 (according to Chothia); or
4) SEQ ID NOs: 463, 470, 465, 466, 469 and 327 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 631 (antibody 4).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 8 and 9 (according to Kabat);
2) SEQ ID NOs: 178, 184, 180, 181, 182 and 9 (according to IMGT);
3) SEQ ID NOs: 323, 330, 325, 326, 182 and 329 (according to Chothia); or
4) SEQ ID NOs: 463, 470, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 632 (antibody 5).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 13, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 1132, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 633 and 632 (antibody 6).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 14, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 471, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 634 and 632 (antibody 7).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 15, 3, 11, 8 and 9 (according to Kabat);
2) SEQ ID NOs: 178, 186, 180, 181, 182 and 9 (according to IMGT);
3) SEQ ID NOs: 323, 332, 325, 326, 182 and 329 (according to Chothia); or
4) SEQ ID NOs: 463, 472, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 635 and 632 (antibody 8).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 16, 3, 11, 8 and 9 (according to Kabat);
2) SEQ ID NOs: 178, 187, 180, 181, 182 and 9 (according to IMGT);
3) SEQ ID NOs: 323, 333, 325, 326, 182 and 329 (according to Chothia); or
4) SEQ ID NOs: 463, 473, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 636 and 632 (antibody 9).

In some embodiments, provided is an antibody or antigen-binding fragment complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 15, 3, 11, 12 and 17 (according to Kabat);
2) SEQ ID NOs: 178, 186, 180, 181, 182 and 17 (according to IMGT);
3) SEQ ID NOs: 323, 332, 325, 326, 182 and 334 (according to Chothia); or
4) SEQ ID NOs: 463, 472, 465, 466, 474 and 334 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 635 and 637 (antibody 10).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 16, 3, 11, 12 and 18 (according to Kabat);
2) SEQ ID NOs: 178, 187, 180, 181, 182 and 18 (according to IMGT);
3) SEQ ID NOs: 323, 333, 325, 326, 182 and 335 (according to Chothia); or
4) SEQ ID NOs: 463, 473, 465, 466, 474 and 335 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 636 and 638 (antibody 11).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1078, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 475, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 639 and 632 (antibody 12).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1079, 3, 11, 8 and 9 (according to Kabat);
2) SEQ ID NOs: 178, 188, 180, 181, 182 and 9 (according to IMGT);
3) SEQ ID NOs: 323, 336, 325, 326, 182 and 329 (according to Chothia); or
4) SEQ ID NOs: 463, 475, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 632 (antibody 13). In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 641 and 632 (antibody 14). In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 644 (antibody 17). In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 645 (antibody 18). In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 646 (antibody 19).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1080, 3, 11, 8 and 9 (according to Kabat);
2) SEQ ID NOs: 178, 189, 180, 181, 182 and 9 (according to IMGT);
3) SEQ ID NOs: 323, 337, 325, 326, 182 and 329 (according to Chothia); or
4) SEQ ID NOs: 463, 476, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ

ID NOs: 642 and 632 (antibody 15).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1080, 3, 11, 8 and 9 (according to Kabat);
2) SEQ ID NOs: 178, 190, 180, 181, 182 and 9 (according to IMGT);
3) SEQ ID NOs: 323, 338, 325, 326, 182 and 329 (according to Chothia); or
4) SEQ ID NOs: 463, 477, 465, 466, 469 and 329 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 643 and 632 (antibody 16).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 20, 21, 22, 23 and 24 (according to Kabat);
2) SEQ ID NOs: 191, 192, 193, 194, 195 and 24 (according to IMGT);
3) SEQ ID NOs: 339, 340, 341, 342, 195 and 1131 (according to Chothia); or
4) SEQ ID NOs: 479, 480, 481, 482, 483 and 1131 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 647 and 648 (antibody 1.17).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 26, 27, 28, 29 and 30 (according to Kabat);
2) SEQ ID NOs: 196, 197, 198, 199, 200 and 30 (according to IMGT);
3) SEQ ID NOs: 344, 345, 346, 347, 200 and 348 (according to Chothia); or
4) SEQ ID NOs: 484, 485, 486, 487, 488 and 348 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 649 and 650 (antibody 1.25).

In some embodiments, provided is an antibody or antigen-binding fragment complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 31, 32, 33, 34, 29 and 35 (according to Kabat);
2) SEQ ID NOs: 201, 202, 203, 199, 200 and 35 (according to IMGT);
3) SEQ ID NOs: 349, 350, 351, 347, 200 and 352 (according to Chothia); or
4) SEQ ID NOs: 489, 490, 491, 487, 492 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 651 and 652 (antibody 1.26).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 36, 37, 28, 29 and 38 (according to Kabat);
2) SEQ ID NOs: 204, 205, 206, 199, 200 and 38 (according to IMGT);
3) SEQ ID NOs: 353, 354, 355, 347, 200 and 356 (according to Chothia); or
4) SEQ ID NOs: 493, 494, 495, 487, 492 and 356 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 653 and 654 (antibody 1.27).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 1082, 39, 28, 29 and 38 (according to Kabat);
2) SEQ ID NOs: 196, 207, 208, 199, 200 and 38 (according to IMGT);
3) SEQ ID NOs: 344, 350, 357, 347, 200 and 356 (according to Chothia); or
4) SEQ ID NOs: 484, 496, 497, 487, 492 and 356 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 655 and 656 (antibody 1.28).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 40, 41, 42, 28, 29 and 35 (according to Kabat);
2) SEQ ID NOs: 209, 210, 211, 199, 200 and 35 (according to IMGT);
3) SEQ ID NOs: 358, 350, 359, 347, 200 and 352 (according to Chothia); or
4) SEQ ID NOs: 498, 499, 500, 487, 492 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 657 and 658 (antibody 1.29).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 43, 39, 28, 29 and 38 (according to Kabat);
2) SEQ ID NOs: 196, 212, 208, 199, 200 and 38 (according to IMGT);
3) SEQ ID NOs: 343, 354, 357, 347, 200 and 356 (according to Chothia); or
4) SEQ ID NOs: 484, 501, 497, 487, 492 and 356 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 659 and 660 (antibody 1.30).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 44, 45, 28, 29 and 35 (according to Kabat);
2) SEQ ID NOs: 196, 213, 214, 199, 200 and 35 (according to IMGT);
3) SEQ ID NOs: 343, 360, 361, 347, 200 and 352 (according to Chothia); or
4) SEQ ID NOs: 484, 502, 503, 487, 492 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 661 and 662 (antibody 1.31).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 46, 47, 28, 29 and 35 (according to Kabat);
2) SEQ ID NOs: 196, 213, 215, 199, 200 and 35 (according to IMGT);
3) SEQ ID NOs: 343, 360, 362, 347, 200 and 352 (according to Chothia); or
4) SEQ ID NOs: 484, 504, 505, 487, 506 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 663 and 664 (antibody 1.32).

In some embodiments, provided is an antibody or antigen-binding fragment complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 48, 49, 28, 29 and 35 (according to Kabat);
2) SEQ ID NOs: 196, 216, 217, 199, 200 and 35 (according to IMGT);
3) SEQ ID NOs: 343, 363, 364, 347, 200 and 352 (according to Chothia); or
4) SEQ ID NOs: 484, 507, 508, 487, 492 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 665 and 666 (antibody 1.33).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 48, 50, 28, 29 and 35 (according to Kabat);
2) SEQ ID NOs: 196, 216, 218, 199, 200 and 35 (according to IMGT);
3) SEQ ID NOs: 343, 363, 365, 347, 200 and 352 (according to Chothia); or
4) SEQ ID NOs: 484, 507, 509, 487, 492 and 352 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 667 and 666 (antibody 1.34).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 51, 52, 53, 54, 55 and 56 (according to Kabat);
2) SEQ ID NOs: 219, 220, 221, 222, 223 and 56 (according to IMGT);
3) SEQ ID NOs: 366, 367, 368, 1084, 223 and 369 (according to Chothia); or
4) SEQ ID NOs: 510, 511, 512, 513, 514 and 369 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 668 and 669 (antibody 1.45).

In some embodiments, provided is an antibody or antigen-binding fragment thereof that specifically binds to CD4 D1 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 51, 57, 53, 58, 55 and 56 (according to Kabat);
2) SEQ ID NOs: 219, 224, 221, 225, 223 and 56 (according to IMGT);
3) SEQ ID NOs: 366, 367, 368, 370, 223 and 369 (according to Chothia); or
4) SEQ ID NOs: 510, 515, 512, 516, 514 and 369 (according to Honegger).

In some embodiments, provided is an antibody or antigen-binding fragment that specifically binds to CD4 D1, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 670 and 671, 672 and 673, or 672 and 671 (antibody 1.46, 1.47 or 1.48).

Antigen Binding Domains that Specifically Bind to CD4 D2

Provided are antibodies or antigen-binding fragments thereof that specifically bind to CD4 D2. Antibodies or antigen-binding fragments thereof that specifically bind to CD4 D2 are useful in combination therapies with molecules that comprise a CD4 D1 extracellular domain, e.g., a CD4 D1-Fc fusion protein, e.g., a molecule comprising CD4 D1.22, described in Chen, et al., J. Virol. (2014) 88(2):1125-1139. For example, antibodies or antigen-binding fragments thereof that specifically bind to CD4 D2 are useful in combination therapies with molecules comprising CD4 D1, e.g., molecules comprising a CD4-Fc fusion protein described, e.g., in certain embodiments, the IL-15v, IL-15Rα Sushi domain-IL-15v fusion protein or CD4-targeted IL-15v, as described herein, is combined or co-administered with. In some embodiments, the antibodies or antigen-binding fragments thereof that specifically bind to CD4 D2, described herein, are insensitive to (i.e., bind to CD4 in the presence and absence of the polymorphism) the amino acid substitutions resulting from CD4 polymorphism variant ID rs11064416 (F123L), wherein the amino acid positions are with reference to SEQ ID NO: 1120.

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of:
1) SEQ ID NOs: 25, 83, 84, 85, 86 and 87; (1.1)
2) SEQ ID NOs: 88, 89, 90, 91, 92 and 93; (1.4)
3) SEQ ID NOs: 94, 95, 96, 97, 98 and 99; (1.5)
4) SEQ ID NOs: 88, 100, 101, 102, 103 and 104; (2.9)
5) SEQ ID NOs: 88, 105, 106, 107, 108 and 93; (2.10)
6) SEQ ID NOs: 88, 109, 110, 111, 108 and 112; (2.11)
7) SEQ ID NOs: 88, 109, 110, 111, 113 and 112; (2.11 VL N50G)
8) SEQ ID NOs: 88, 114, 106, 107, 108 and 115; (2.12)
9) SEQ ID NOs: 88, 114, 106, 107, 113 and 115; (2.12 VL N50G)
10) SEQ ID NOs: 88, 116, 117, 118, 108 and 119; (2.13)
11) SEQ ID NOs: 120, 121, 122, 102, 113 and 123; (2.14) or
12) SEQ ID NOs: 88, 124, 122, 125, 113 and 104; (2.15).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to IMGT) of:
1) SEQ ID NOs: 246, 247, 248, 249, 250 and 87; (1.1)
2) SEQ ID NOs: 251, 252, 253, 254, 255 and 93; (1.4)
3) SEQ ID NOs: 256, 257, 258, 259, 255 and 99; (1.5)
4) SEQ ID NOs: 251, 260, 261, 262, 263 and 104; (2.9)
5) SEQ ID NOs: 251, 264, 265, 266, 267 and 93; (2.10)
6) SEQ ID NOs: 251, 268, 269, 270, 267 and 112; (2.11)
7) SEQ ID NOs: 251, 268, 269, 270, 263 and 112; (2.11 VL N50G)
8) SEQ ID NOs: 251, 271, 265, 266, 267 and 115; (2.12)
9) SEQ ID NOs: 251, 271, 265, 266, 263 and 115; (2.12 VL N50G)
10) SEQ ID NOs: 251, 272, 273, 274, 267 and 119; (2.13)
11) SEQ ID NOs: 275, 276, 277, 262, 263 and 123; (2.14) or
12) SEQ ID NOs: 251, 278, 277, 279, 263 and 104; (2.15).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Chothia) of:
1) SEQ ID NOs: 390, 391, 392, 393, 250 and 394; (1.1)
2) SEQ ID NOs: 395, 396, 397, 398, 255 and 399; (1.4)
3) SEQ ID NOs: 400, 401, 402, 403, 255 and 404; (1.5)
4) SEQ ID NOs: 395, 405, 406, 407, 263 and 408; (2.9)
5) SEQ ID NOs: 395, 409, 410, 411, 267 and 399; (2.10)
6) SEQ ID NOs: 395, 412, 413, 414, 267 and 399; (2.11)
7) SEQ ID NOs: 395, 412, 413, 414, 263 and 399; (2.11 VL N50G)
8) SEQ ID NOs: 395, 415, 410, 411, 267 and 408; (2.12)
9) SEQ ID NOs: 395, 415, 410, 411, 263 and 408; (2.12 VL N50G)
10) SEQ ID NOs: 395, 416, 417, 418, 267 and 419; (2.13)
11) SEQ ID NOs: 420, 421, 422, 407, 263 and 408; (2.14) or
12) SEQ ID NOs: 395, 423, 422, 424, 263 and 408; (2.15).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Honegger) of:
1) SEQ ID NOs: 536, 537, 538, 539, 540 and 394; (1.1)
2) SEQ ID NOs: 541, 542, 543, 544, 545 and 399; (1.4)
3) SEQ ID NOs: 546, 547, 548, 549, 550 and 404; (1.5)
4) SEQ ID NOs: 541, 551, 552, 553, 554 and 408; (2.9)
5) SEQ ID NOs: 541, 555, 556, 557, 558 and 399; (2.10)
6) SEQ ID NOs: 541, 559, 560, 561, 562 and 399; (2.11)
7) SEQ ID NOs: 541, 559, 560, 561, 563 and 399; (2.11 VL N50G)
8) SEQ ID NOs: 541, 564, 556, 557, 558 and 408; (2.12)
9) SEQ ID NOs: 541, 564, 556, 557, 565 and 408; (2.12 VL N50G)
10) SEQ ID NOs: 541, 566, 567, 568, 569 and 419; (2.13)
11) SEQ ID NOs: 570, 571, 572, 553, 565 and 408; (2.14) or
12) SEQ ID NOs: 541, 573, 572, 574, 575 and 408; (2.15).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 682 and 683; (1.1)
2) SEQ ID NOs: 684 and 685; (1.4)
3) SEQ ID NOs: 686 and 687; (1.5)
4) SEQ ID NOs: 688 and 689; (2.9)
5) SEQ ID NOs: 690 and 691; (2.10)
6) SEQ ID NOs: 692 and 693; (2.11)
7) SEQ ID NOs: 692 and 694; (2.11 VL N50G)
8) SEQ ID NOs: 695 and 696; (2.12)
9) SEQ ID NOs: 695 and 697; (2.12 VL N50G)
10) SEQ ID NOs: 698 and 699; (2.13)
11) SEQ ID NOs: 700 and 701; (2.14) or
12) SEQ ID NOs: 702 and 703; (2.15).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 83, 84, 85, 86 and 87 (according to Kabat);
2) SEQ ID NOs: 246, 247, 248, 249, 250 and 87 (according to IMGT);
3) SEQ ID NOs: 390, 391, 392, 393, 250 and 394 (according to Chothia); or
4) SEQ ID NOs: 536, 537, 538, 539, 540 and 394 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 682 and 683 (antibody SCT1.1).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 89, 90, 91, 92 and 93 (according to Kabat);
2) SEQ ID NOs: 251, 252, 253, 254, 255 and 93 (according to IMGT);
3) SEQ ID NOs: 395, 396, 397, 398, 255 and 399 (according to Chothia); or
4) SEQ ID NOs: 541, 542, 543, 544, 545 and 399 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 684 and 685 (antibody SCT1.4).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 94, 95, 96, 97, 98 and 99 (according to Kabat);
2) SEQ ID NOs: 256, 257, 258, 259, 255 and 99 (according to IMGT);
3) SEQ ID NOs: 400, 401, 402, 403, 255 and 404 (according to Chothia); or
4) SEQ ID NOs: 546, 547, 548, 549, 550 and 404 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 686 and 687 (antibody SCT1.5).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 100, 101, 102, 103 and 104 (according to Kabat);
2) SEQ ID NOs: 251, 260, 261, 262, 263 and 104 (according to IMGT);
3) SEQ ID NOs: 395, 405, 406, 407, 263 and 408 (according to Chothia); or
4) SEQ ID NOs: 541, 551, 552, 553, 554 and 408 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 688 and 689 (antibody SCT2.9).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 105, 106, 107, 108 and 93 (according to Kabat);
2) SEQ ID NOs: 251, 264, 265, 266, 267 and 93 (according to IMGT);
3) SEQ ID NOs: 395, 409, 410, 411, 267 and 399 (according to Chothia); or
4) SEQ ID NOs: 541, 555, 556, 557, 558 and 399 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 690 and 691 (antibody SCT2.10).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 109, 110, 111, 108 and 112 (according to Kabat);
2) SEQ ID NOs: 251, 268, 269, 270, 267 and 112 (according to IMGT);
3) SEQ ID NOs: 395, 412, 413, 414, 267 and 399 (according to Chothia); or
4) SEQ ID NOs: 541, 559, 560, 561, 562 and 399 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 692 and 693 (antibody SCT2.11).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 109, 110, 111, 113 and 112 (according to Kabat);
2) SEQ ID NOs: 251, 268, 269, 270, 263 and 112 (according to IMGT);
3) SEQ ID NOs: 395, 412, 413, 414, 263 and 399 (according to Chothia); or
4) SEQ ID NOs: 541, 559, 560, 561, 563 and 399 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 692 and 694 (antibody SCT2.11 VL N50G).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 114, 106, 107, 108 and 115 (according to Kabat);
2) SEQ ID NOs: 251, 271, 265, 266, 267 and 115 (according to IMGT);
3) SEQ ID NOs: 395, 415, 410, 411, 267 and 408 (according to Chothia); or
4) SEQ ID NOs: 541, 564, 556, 557, 558 and 408 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 695 and 696 (antibody SCT2.12).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 114, 106, 107, 113 and 115 (according to Kabat);
2) SEQ ID NOs: 251, 271, 265, 266, 263 and 115 (according to IMGT);
3) SEQ ID NOs: 395, 415, 410, 411, 263 and 408 (according to Chothia); or
4) SEQ ID NOs: 541, 564, 556, 557, 565 and 408 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 695 and 697 (antibody SCT2.12 VL N50G).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 116, 117, 118, 108 and 119 (according to Kabat);
2) SEQ ID NOs: 251, 272, 273, 274, 267 and 119 (according to IMGT);
3) SEQ ID NOs: 395, 416, 417, 418, 267 and 419 (according to Chothia); or
4) SEQ ID NOs: 541, 566, 567, 568, 569 and 419 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 698 and 699 (antibody SCT2.13).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 120, 121, 122, 102, 113 and 123 (according to Kabat);
2) SEQ ID NOs: 275, 276, 277, 262, 263 and 123 (according to IMGT);
3) SEQ ID NOs: 420, 421, 422, 407, 263 and 408 (according to Chothia); or
4) SEQ ID NOs: 570, 571, 572, 553, 565 and 408 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 700 and 701 (antibody SCT2.14).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
5) SEQ ID NOs: 88, 124, 122, 125, 113 and 104 (according to Kabat);
6) SEQ ID NOs: 251, 278, 277, 279, 263 and 104 (according to IMGT);
7) SEQ ID NOs: 395, 423, 422, 424, 263 and 408 (according to Chothia); or
8) SEQ ID NOs: 541, 573, 572, 574, 575 and 408 (according to Honegger).

In some embodiments, provided is an antibody or antigen-binding fragment that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 702 and 703 (antibody SCT2.15).

Antigen Binding Domains that Specifically Bind to One or Both of CD4 D2 and CD4 D3

Provided are antibodies or antigen-binding fragments thereof that specifically bind to one or both of CD4 D2 and CD4 D3. Antibodies or antigen-binding fragments thereof that specifically bind to one or both of CD4 D2 and CD4 D3 are useful in combination therapies with molecules that comprise a CD4 D1 extracellular domain, e.g., a CD4 D1-Fc fusion protein, e.g., a molecule comprising CD4 D1.22, described in Chen, et al., J. Virol. (2014) 88(2):1125-1139. For example, antibodies or antigen-binding fragments thereof that specifically bind to one or both of CD4 D2 and CD4 D3 are useful in combination therapies with molecules comprising CD4 D1, e.g., molecules comprising a CD4-Fc fusion protein described, e.g., in WO 2011/146891, WO 2019/183387, WO 2022/046644 (e.g., amtabafusp alfa (GS-8588)) and WO 2024/094690. In some embodiments, the antibodies or antigen-binding fragments thereof that specifically bind to one or both of CD4 D2 and CD4 D3, described herein, are insensitive to the amino acid substitutions resulting from one or more of CD4 polymorphism variant IDs rs28919570 (R265W), rs11064419 (F227S or F227C) and rs11064416 (F123L), wherein the amino acid positions are with reference to SEQ ID NO: 1120.

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of:
1) SEQ ID NOs: 126, 127, 128, 129, 130 and 99; (1.6)
2) SEQ ID NOs: 126, 131, 132, 129, 133 and 99; (1.7)
3) SEQ ID NOs: 126, 134, 135, 136, 133 and 99; (1.8)
4) SEQ ID NOs: 126, 137, 128, 129, 138 and 99; (1.9)
5) SEQ ID NOs: 126, 139, 128, 140, 141 and 99; (1.10)
6) SEQ ID NOs: 142, 143, 128, 145, 141 and 99; (1.19)
7) SEQ ID NOs: 126, 146, 147, 140, 141 and 99; (2.2) or
8) SEQ ID NOs: 148, 149, 150, 129, 151 and 99; (2.3).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to IMGT) of:
1) SEQ ID NOs: 280, 281, 282, 283, 284 and 99; (1.6)
2) SEQ ID NOs: 280, 285, 286, 283, 284 and 99; (1.7)
3) SEQ ID NOs: 280, 287, 288, 283, 284 and 99; (1.8)
4) SEQ ID NOs: 289, 290, 282, 283, 284 and 99; (1.9)
5) SEQ ID NOs: 289, 291, 292, 293, 255 and 99; (1.10)
6) SEQ ID NOs: 294, 295, 296, 297, 255 and 99; (1.19)
7) SEQ ID NOs: 289, 298, 299, 293, 255 and 99; (2.2) or
8) SEQ ID NOs: 289, 300, 301, 283, 284 and 99; (2.3).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Chothia) of:
1) SEQ ID NOs: 425, 426, 427, 428, 284 and 404; (1.6)
2) SEQ ID NOs: 425, 426, 429, 428, 284 and 404; (1.7)
3) SEQ ID NOs: 425, 426, 430, 428, 284 and 404; (1.8)
4) SEQ ID NOs: 431, 432, 427, 428, 284 and 404; (1.9)
5) SEQ ID NOs: 431, 433, 427, 434, 255 and 404; (1.10)
6) SEQ ID NOs: 431, 435, 436, 437, 255 and 404; (1.19)
7) SEQ ID NOs: 431, 438, 439, 434, 255 and 404; (2.2) or
8) SEQ ID NOs: 431, 438, 440, 428, 284 and 404; (2.3).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Honegger) of:
1) SEQ ID NOs: 576, 577, 578, 579, 580 and 404; (1.6)
2) SEQ ID NOs: 576, 581, 582, 579, 583 and 404; (1.7)
3) SEQ ID NOs: 576, 584, 585, 586, 583 and 404; (1.8)
4) SEQ ID NOs: 587, 588, 578, 579, 589 and 404; (1.9)
5) SEQ ID NOs: 587, 590, 578, 591, 592 and 404; (1.10)
6) SEQ ID NOs: 593, 594, 595, 596, 597 and 404; (1.19)
7) SEQ ID NOs: 587, 598, 599, 591, 597 and 404; (2.2) or
8) SEQ ID NOs: 587, 600, 601, 579, 602 and 404; (2.3).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 704 and 705; (1.6)
2) SEQ ID NOs: 706 and 707; (1.7)
3) SEQ ID NOs: 708 and 709; (1.8)
4) SEQ ID NOs: 710 and 711; (1.9)
5) SEQ ID NOs: 712 and 713; (1.10)
6) SEQ ID NOs: 714 and 715; (1.19)
7) SEQ ID NOs: 716 and 717; (2.2) or
8) SEQ ID NOs: 718 and 719; (2.3).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 127, 128, 129, 130 and 99 (according to Kabat);
2) SEQ ID NOs: 280, 281, 282, 283, 284 and 99 (according to IMGT);
3) SEQ ID NOs: 425, 426, 427, 428, 284 and 404 (according to Chothia); or
4) SEQ ID NOs: 576, 577, 578, 579, 580 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 704 and 705 (antibody SCT1.6).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 131, 132, 129, 133 and 99 (according to Kabat);
2) SEQ ID NOs: 280, 285, 286, 283, 284 and 99 (according to IMGT);
3) SEQ ID NOs: 425, 426, 429, 428, 284 and 404 (according to Chothia); or
4) SEQ ID NOs: 576, 581, 582, 579, 583 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 706 and 707 (antibody SCT1.7).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 134, 135, 136, 133 and 99 (according to Kabat);
2) SEQ ID NOs: 280, 287, 288, 283, 284 and 99 (according to IMGT);
3) SEQ ID NOs: 425, 426, 429, 428, 284 and 404 (according to Chothia); or
4) SEQ ID NOs: 576, 584, 585, 586, 583 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 708 and 709 (antibody SCT1.8).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 137, 128, 129, 138 and 99 (according to Kabat);
2) SEQ ID NOs: 289, 290, 282, 283, 284 and 99 (according to IMGT);
3) SEQ ID NOs: 431, 432, 427, 428, 284 and 404 (according to Chothia); or
4) SEQ ID NOs: 587, 588, 578, 579, 589 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 710 and 711 (antibody SCT1.9).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 139, 128, 140, 141 and 99 (according to Kabat);
2) SEQ ID NOs: 289, 291, 292, 293, 255 and 99 (according to IMGT);
3) SEQ ID NOs: 431, 433, 427, 434, 255 and 404 (according to Chothia); or
4) SEQ ID NOs: 587, 590, 578, 591, 592 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 712 and 713 (antibody SCT1.10).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 142, 143, 128, 145, 141 and 99 (according to Kabat);
2) SEQ ID NOs: 294, 295, 296, 297, 255 and 99 (according to IMGT);
3) SEQ ID NOs: 431, 435, 436, 437, 255 and 404 (according to Chothia); or
4) SEQ ID NOs: 593, 594, 595, 596, 597 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 714 and 715 (antibody SCT1.19).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 146, 147, 140, 141 and 99 (according to Kabat);
2) SEQ ID NOs: 289, 298, 299, 293, 255 and 99 (according to IMGT);
3) SEQ ID NOs: 431, 438, 439, 434, 255 and 404 (according to Chothia); or
4) SEQ ID NOs: 587, 598, 599, 591, 597 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 716 and 717 (antibody SCT2.2).

In some embodiments, provided is an antibody or antigen-binding fragment thereof that specifically binds to one or both of CD4 D2 and CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 148, 149, 150, 129, 151 and 99 (according to Kabat);
2) SEQ ID NOs: 289, 300, 301, 283, 284 and 99 (according to IMGT);
3) SEQ ID NOs: 431, 438, 440, 428, 284 and 404 (according to Chothia); or
4) SEQ ID NOs: 587, 600, 601, 579, 602 and 404 (according to Honegger).

In some embodiments, provided is an antibody or antigen-binding fragment that specifically binds to one or both of CD4 D2 and CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 718 and 719 (antibody SCT2.3).

### Antigen Binding Domains That Specifically Bind to CD4 D3

Provided are antibodies or antigen-binding fragments thereof that specifically bind to CD4 D3. Antibodies or antigen-binding fragments thereof that specifically bind to CD4 D3 are useful in combination therapies with molecules that comprise a CD4 D1 or a CD4 D1D2 extracellular domain, e.g., a CD4 D1-Fc fusion protein, e.g., a molecule comprising CD4 D1.22 (with or without a D2), described in Chen, et al., J. Virol. (2014) 88(2):1125-1139. For example, antibodies or antigen-binding fragments thereof that specifically bind to CD4 D3 are useful in combination therapies with molecules comprising CD4 D1 or CD4 D1D2 described, e.g., in U.S. Pat. No. 7,368,114; and Intl. Publ. Nos. WO 2011/146891, WO 2019/183387, WO 2022/046644 (e.g., amtabafusp alfa (GS-8588)) and WO 2024/094690. In some embodiments, the antibodies or antigen-binding fragments thereof that specifically bind to CD4 D3, described herein, are insensitive to the amino acid substitutions resulting from one or more of CD4 polymorphism variant IDs rs28919570 (R265W) and rs11064419 (F227S or F227C), wherein the amino acid positions are with reference to SEQ ID NO: 1120. In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120. In some embodiments, the antibodies or antigen-binding fragments thereof that specifically bind to CD4 D3 bind to CD4 D3 with a binding equilibrium dissociation constant (K_{D}) of lower than 1 nM, e.g., lower than 100 pM, lower than 90 pM, lower than 80 pM, lower than 70 pM, lower than 60 pM, lower than 50 pM, lower than 40 pM, or lower than 30 pM.

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of:
1) SEQ ID NOs: 158, 159, 160, 145, 161 and 162; (1.20)
2) SEQ ID NOs: 163, 164, 160, 165, 166 and 162; (1.21)
3) SEQ ID NOs: 163, 164, 160, 165, 161 and 162; (1.22)
4) SEQ ID NOs: 158, 167, 168, 145, 98 and 169; (1.23)
5) SEQ ID NOs: 170, 171, 172, 173, 141 and 99; (2.1) or
6) SEQ ID NOs: 25, 174, 175, 176, 29 and 177; (2.8).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to IMGT) of:
1) SEQ ID NOs: 306, 307, 1083, 297, 308 and 162; (1.20)
2) SEQ ID NOs: 309, 310, 311, 312, 313 and 162; (1.21)
3) SEQ ID NOs: 309, 310, 311, 312, 308 and 162; (1.22)
4) SEQ ID NOs: 314, 315, 316, 297, 255 and 169; (1.23)
5) SEQ ID NOs: 317, 318, 319, 320, 255 and 99; (2.1) or
6) SEQ ID NOs: 196, 321, 322, 199, 200 and 177; (2.8).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Chothia) of:
1) SEQ ID NOs: 446, 447, 448, 437, 308 and 449; (1.20)
2) SEQ ID NOs: 450, 451, 448, 452, 313 and 449; (1.21)
3) SEQ ID NOs: 450, 451, 448, 452, 308 and 449; (1.22)
4) SEQ ID NOs: 453, 454, 455, 437, 255 and 456; (1.23)
5) SEQ ID NOs: 457, 458, 459, 460, 255 and 404; (2.1) or
6) SEQ ID NOs: 343, 350, 461, 347, 200 and 462; (2.8).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Honegger) of:
1) SEQ ID NOs: 608, 609, 610, 596, 611 and 449; (1.20)
2) SEQ ID NOs: 612, 613, 610, 614, 615 and 449; (1.21)
3) SEQ ID NOs: 612, 613, 610, 614, 611 and 449; (1.22)
4) SEQ ID NOs: 616, 617, 618, 596, 550 and 456; (1.23)
5) SEQ ID NOs: 619, 620, 621, 622, 597 and 404; (2.1) or
6) SEQ ID NOs: 484, 623, 624, 487, 492 and 462; (2.8).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 722 and 723; (1.20)
2) SEQ ID NOs: 724 and 725; (1.21)
3) SEQ ID NOs: 724 and 726; (1.22)
4) SEQ ID NOs: 727 and 728; (1.23)
5) SEQ ID NOs: 727 and 729; (1.23 VL C36Y)
6) SEQ ID NOs: 730 and 731; (2.1) or
7) SEQ ID NOs: 732 and 733; (2.8).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 158, 159, 160, 145, 161 and 162 (according to Kabat);
2) SEQ ID NOs: 306, 307, 1083, 297, 308 and 162 (according to IMGT);
3) SEQ ID NOs: 446, 447, 448, 437, 308 and 449 (according to Chothia); or
4) SEQ ID NOs: 608, 609, 610, 596, 611 and 449 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 722 and 723 (antibody SCT1.20).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 163, 164, 160, 165, 166 and 162 (according to Kabat);
2) SEQ ID NOs: 309, 310, 311, 312, 313 and 162 (according to IMGT);
3) SEQ ID NOs: 450, 451, 448, 452, 313 and 449 (according to Chothia); or
4) SEQ ID NOs: 612, 613, 610, 614, 615 and 449 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 724 and 725 (antibody SCT1.21).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 163, 164, 160, 165, 161 and 162 (according to Kabat);
2) SEQ ID NOs: 309, 310, 311, 312, 308 and 162 (according to IMGT);
3) SEQ ID NOs: 450, 451, 448, 452, 308 and 449 (according to Chothia); or
4) SEQ ID NOs: 612, 613, 610, 614, 611 and 449 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 724 and 726 In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120 (antibody SCT1.22).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 158, 167, 168, 145, 98 and 169 (according to Kabat);
2) SEQ ID NOs: 314, 315, 316, 297, 255 and 169 (according to IMGT);
3) SEQ ID NOs: 453, 454, 455, 437, 255 and 456 (according to Chothia); or
4) SEQ ID NOs: 616, 617, 618, 596, 550 and 456 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 727 and 728 or 727 and 729 (antibody SCT1.23 and SCT1.23 VL C36Y).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 170, 171, 172, 173, 141 and 99 (according to Kabat);
2) SEQ ID NOs: 317, 318, 319, 320, 255 and 99 (according to IMGT);
3) SEQ ID NOs: 457, 458, 459, 460, 255 and 404 (according to Chothia); or
4) SEQ ID NOs: 619, 620, 621, 622, 597 and 404 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 730 and 731 (antibody SCT2.1).

In some embodiments, provided is an antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 174, 175, 176, 29 and 177 (according to Kabat);
2) SEQ ID NOs: 196, 321, 322, 199, 200 and 177 (according to IMGT);
3) SEQ ID NOs: 343, 350, 461, 347, 200 and 462 (according to Chothia); or
4) SEQ ID NOs: 484, 623, 624, 487, 492 and 462 (according to Honegger).

In some embodiments, provided is an antibody or antigen-binding fragment that specifically binds to CD4 D3, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 732 and 733 (antibody SCT2.8).

As appropriate or desired, alternative framework regions can be grafted onto the CDRs (e.g., according to Kabat) that maintain the desired function of the anti-CD4 antigen binding domains. Applicable approaches for humanization and re-humanization (e.g., grafting alternative framework regions onto selected CDRs) that can be used for humanization or re-humanization of the herein described anti-CD4 antigen binding domains are described e.g., in Gupta, et al., J Biol Chem (2024) 300(1):105555; Tennenhouse, et al., Nat Biomed Eng. (2024) 8(1):30-44; Jiacomini, et al., Int J Biol Macromol. (2022) 216:465-474; Marks, et al., Bioinformatics (2021) 37(22):4041-4047; Aubrey, et al., Methods Mol Biol (2019) 1904:231-252; Kuramochi, et al., Methods Mol Biol (2019) 1904:213-230; Apgar, et al., MAbs (2016) 8(7):1302-1318; Choi, et al., MAbs (2015) 7(6):1045-57; Hanf, et al., Methods (2014) 65(1):68-76; Ahmadzadeh, et al., Monoclon Antib Immunodiagn Immunother (2014) 33(2):67-73; Safdari, et al., Biotechnol Genet Eng Rev (2013) 29:175-86; Kim, et al., Methods Mol Biol. (2012) 907:237-45; Almagro, et al., Front Biosci. (2008) 13:1619-33; Kashmiri, et al., Methods (2005) 36(1):25-34, Foote and Winter, J Mol Biol (1992) 224(2):487-99, as well as in U.S. Pat. Nos. 8,323,651; 8,324,350; 9,550,986; and 10,174,121, which are herein incorporated by reference in their entireties for all purposes. A software platform useful for antibody design, humanization, and humanness evaluation that can be used for humanization or re-humanization of the herein described anti-CD4 antigen binding domains is BioPhi (reviewed in Prihoda, et al. MAbs (2022) 14(1):2020203 and accessible at biophi.dichlab.org). In some embodiments, the alternative framework regions useful for grafting have at least 90%, at least 91%, at least 92%, at least 93%, at least 94%, at least 95%, at least 96%, at least 97%, at least 98%, or at least 99% amino acid sequence identity to a reference framework region amino acid sequence. In some embodiments, the humanized or re-humanized VH and VL amino acid sequences have at least 90%, at least 91%, at least 92%, at least 93%, at least 94%, at least 95%, at least 96%, at least 97%, at least 98%, or at least 99% identity to reference VH and VL amino acid sequences.

Illustrative sequences of the CDRs of the anti-CD4 antigen binding domains are provided in Tables A1-A4. Illustrative sequences of the VH and VL of the anti-CD4 antigen binding domains described herein are provided in Table B.

TABLE A1
CDRs for illustrative anti-CD4 binding domains (Kabat)

| | | | | | | |
|---|---|---|---|---|---|---|
| Ab | VH - | | | | | |
| Name | CDR1 | VH - CDR2 | VH - CDR3 | VL - CDR1 | VL - CDR2 | VL - CDR3 |

Binds to CD4 D1

| | | | | | | |
|---|---|---|---|---|---|---|
| 1 | NYAMS | SINX₁X₂GSTFYX₃DSVKX₄ | HYGGSYDPMDY | X₅ASQDINKYIA | YTSX₆LHP | LQYDNPLX₇T |
| | SEQ ID | X₁ is A, D or E | SEQ ID NO: 3 | X₅ is K or R | X₆ is I or T | X₇ is F, Q |
| | NO: 1 | X₂ is A, G, W or Y | | SEQ ID NO: 4 | SEQ ID NO: 5 | or Y |
| | | X₃ is A or P | | | | SEQ ID NO: 6 |
| | | X₄ is G or S | | | | |
| | | SEQ ID NO: 2 | | | | |
| 2 | NYAMS | SINX₁X₂GSTFYX₃DSVKX₄ | HYGGSYDPMDY | X₅ASQDINKYIA | YTSTLHP | LQYDNPLYT |
| | SEQ ID | X₁ is A, D or E | SEQ ID NO: 3 | X₅ is K or R | SEQ ID NO: 8 | SEQ ID NO: 9 |
| | NO: 1 | X₂ is A, or G | | SEQ ID NO: 4 | | |
| | | X₃ is A or P | | | | |
| | | X₄ is G or S | | | | |
| | | SEQ ID NO: 7 | | | | |
| 3 | NYAMS | SINEX₂GSTFYADSVKS | HYGGSYDPMDY | RASQDINKYIA | YTSX₆LHP | LQYDNPLX₇T |
| | SEQ ID | X₂ is W or Y | SEQ ID NO: 3 | SEQ ID NO: 11 | X₆ is I or T | X₇ is F, Q, Y |
| | NO: 1 | SEQ ID NO: 10 | | | SEQ ID NO: 5 | SEQ ID NO: 6 |
| 4 | NYAMS | SINEX₂GSTFYADSVKS | HYGGSYDPMDY | RASQDINKYIA | YTSILHP | LQYDNPLX₇T |
| | SEQ ID | X₂ is W or Y | SEQ ID NO: 3 | SEQ ID NO: 11 | SEQ ID NO: 12 | X₇ is F, Q, Y |
| | NO: 1 | SEQ ID NO: 10 | | | | SEQ ID NO: 6 |
| 5 | NYAMS | SINEX₂GSTFYADSVKS | HYGGSYDPMDY | RASQDINKYIA | YTSTLHP | LQYDNPLYT |
| | SEQ ID | X₂ is W or Y | SEQ ID NO: 3 | SEQ ID NO: 11 | SEQ ID NO: 8 | SEQ ID NO: 9 |
| | NO: 1 | SEQ ID NO: 10 | | | | |
| 6 | NYAMS | SINEGGSTFYADSVKX₄ | HYGGSYDPMDY | RASQDINKYIA | YTSTLHP | LQYDNPLYT |
| | SEQ ID | X₄ is G or S | SEQ ID NO: 3 | SEQ ID NO: 11 | SEQ ID NO: 8 | SEQ ID NO: 9 |
| | NO: 1 | SEQ ID NO: 13 | | | | |
| 7 | NYAMS | SINEGGSTFYADSVKS | HYGGSYDPMDY | RASQDINKYIA | YTSTLHP | LQYDNPLYT |
| | SEQ ID | SEQ ID NO: 14 | SEQ ID NO: 3 | SEQ ID NO: 11 | SEQ ID NO: 8 | SEQ ID NO: 9 |
| | NO: 1 | | | | | |
| 8 | NYAMS | SINEWGSTFYADSVKS | HYGGSYDPMDY | RASQDINKYIA | YTSTLHP | LQYDNPLYT |
| | SEQ ID | SEQ ID NO: 15 | SEQ ID NO: 3 | SEQ ID NO: 11 | SEQ ID NO: 8 | SEQ ID NO: 9 |
| | NO: 1 | | | | | |
| 9 | NYAMS | SINEYGSTFYADSVKS | HYGGSYDPMDY | RASQDINKYIA | YTSTLHP | LQYDNPLYT |
| | SEQ ID | SEQ ID NO: 16 | SEQ ID NO: 3 | SEQ ID NO: 11 | SEQ ID NO: 8 | SEQ ID NO: 9 |
| | NO: 1 | | | | | |
| 10 | NYAMS | SINEWGSTFYADSVKS | HYGGSYDPMDY | RASQDINKYIA | YTSILHP | LQYDNPLQT |
| | SEQ ID | SEQ ID NO: 15 | SEQ ID NO: 3 | SEQ ID NO: 11 | SEQ ID NO: 12 | SEQ ID NO: 17 |
| | NO: 1 | | | | | |
| 11 | NYAMS | SINEYGSTFYADSVKS | HYGGSYDPMDY | RASQDINKYIA | YTSILHP | LQYDNPLFT |
| | SEQ ID | SEQ ID NO: 16 | SEQ ID NO: 3 | SEQ ID NO: 11 | SEQ ID NO: 12 | SEQ ID NO: 18 |
| | NO: 1 | | | | | |
| 12 | NYAMS | SINEGGSTFYADSVKG | HYGGSYDPMDY | RASQDINKYIA | YTSTLHP | LQYDNPLYT |
| | SEQ ID | SEQ ID NO: 1078 | SEQ ID NO: 3 | SEQ ID NO: 11 | SEQ ID NO: 8 | SEQ ID NO: 9 |
| | NO: 1 | | | | | |
| 13 | NYAMS | SINDGGSTFYADSVKG | HYGGSYDPMDY | RASQDINKYIA | YTSTLHP | LQYDNPLYT |
| | SEQ ID | SEQ ID NO: 1079 | SEQ ID NO: 3 | SEQ ID NO: 11 | SEQ ID NO: 8 | SEQ ID NO: 9 |
| | NO: 1 | | | | | |
| 14 | NYAMS | SINDGGSTFYADSVKG | HYGGSYDPMDY | RASQDINKYIA | YTSTLHP | LQYDNPLYT |
| | SEQ ID | SEQ ID NO: 1079 | SEQ ID NO: 3 | SEQ ID NO: 11 | SEQ ID NO: 8 | SEQ ID NO: 9 |
| | NO: 1 | | | | | |
| 15 | NYAMS | SINAGGSTFYADSVKG | HYGGSYDPMDY | RASQDINKYIA | YTSTLHP | LQYDNPLYT |
| | SEQ ID | SEQ ID NO: 1080 | SEQ ID NO: 3 | SEQ ID NO: 11 | SEQ ID NO: 8 | SEQ ID NO: 9 |
| | NO: 1 | | | | | |
| 16 | NYAMS | SINDAGSTFYADSVKG | HYGGSYDPMDY | RASQDINKYIA | YTSTLHP | LQYDNPLYT |
| | SEQ ID | SEQ ID NO: 1081 | SEQ ID NO: 3 | SEQ ID NO: 11 | SEQ ID NO: 8 | SEQ ID NO: 9 |
| | NO: 1 | | | | | |
| 17 | NYAMS | SINDGGSTFYADSVKG | HYGGSYDPMDY | RASQDINKYIA | YTSTLHP | LQYDNPLYT |
| | SEQ ID | SEQ ID NO: 1079 | SEQ ID NO: 3 | SEQ ID NO: 11 | SEQ ID NO: 8 | SEQ ID NO: 9 |
| | NO: 1 | | | | | |
| 18 | NYAMS | SINDGGSTFYADSVKG | HYGGSYDPMDY | RASQDINKYIA | YTSTLHP | LQYDNPLYT |
| | SEQ ID | SEQ ID NO: 1079 | SEQ ID NO: 3 | SEQ ID NO: 11 | SEQ ID NO: 8 | SEQ ID NO: 9 |
| | NO: 1 | | | | | |
| 19 | NYAMS | SINDGGSTFYADSVKG | HYGGSYDPMDY | RASQDINKYIA | YTSTLHP | LQYDNPLYT |
| | SEQ ID | SEQ ID NO: 1079 | SEQ ID NO: 3 | SEQ ID NO: 11 | SEQ ID NO: 8 | SEQ ID NO: 9 |
| | NO: 1 | | | | | |
| SCT1. | DYGMS | GINWNGDSTGYADSVRG | DGAIGGMDV | RASQDIRDDLG | SASTLQS | LQDYNYPWT |
| 17 | SEQ ID | SEQ ID NO: 20 | SEQ ID NO: 21 | SEQ ID NO: 22 | SEQ ID NO: 23 | SEQ ID NO: 24 |
| | NO: 19 | | | | | |
| SCT1. | ELSMH | YFDPRGGETIYAQKFQG | GGDRDYYYYYMDV | KASQDIDDDMN | EATTLVP | LQHDSFPYT |
| 25 | SEQ ID | SEQ ID NO: 26 | SEQ ID NO: 27 | SEQ ID NO: 28 | SEQ ID NO: 29 | SEQ ID NO: 30 |
| | NO: 25 | | | | | |
| SCT1. | KLSMH | FFDPRDGERIYAQKFQG | GGDYDYYYYYMGV | KASQDIDDDLN | EATTLVP | LQHDNFPYT |
| 26 | SEQ ID | SEQ ID NO: 32 | SEQ ID NO: 33 | SEQ ID NO: 34 | SEQ ID NO: 29 | SEQ ID NO: 35 |
| | NO: 31 | | | | | |
| SCT1. | ELSMH | SFHPRDDEIIYAQKFOG | GGNPDYYFYYMDV | KASQDIDDDMN | EATTLVP | LQHDNFPLT |
| 27 | SEQ ID | SEQ ID NO: 36 | SEQ ID NO: 37 | SEQ ID NO: 28 | SEQ ID NO: 29 | SEQ ID NO: 38 |
| | NO: 25 | | | | | |
| SCT1. | ELSMH | SFHPRDGETIYAQKFQG | GGNKDYYFYYMDV | KASQDIDDDMN | EATTLVP | LQHDNFPLT |
| 28 | SEQ ID | SEQ ID NO: 1082 | SEQ ID NO: 39 | SEQ ID NO: 28 | SEQ ID NO: 29 | SEQ ID NO: 38 |
| | NO: 25 | | | | | |
| SCT1. | ALSMH | YFNPRDGETIYAQKFQG | GGDSDYYYYYMDV | KASQDIDDDMN | EATTLVP | LQHDNFPYT |
| 29 | SEQ ID | SEQ ID NO: 41 | SEQ ID NO: 42 | SEQ ID NO: 28 | SEQ ID NO: 29 | SEQ ID NO: 35 |
| | NO: 40 | | | | | |
| SCT1. | ELSMH | YFNPRDDETIYTQKFOG | GGNKDYYFYYMDV | KASQDIDDDMN | EATTLVP | LQHDNFPLT |
| 30 | SEQ ID | SEQ ID NO: 43 | SEQ ID NO: 39 | SEQ ID NO: 28 | SEQ ID NO: 29 | SEQ ID NO: 38 |
| | NO: 25 | | | | | |
| SCT1. | ELSMH | VFDPKYGETTYAQKFQD | GGNYEYFYYYMDV | KASQDIDDDMN | EATTLVP | LQHDNFPYT |
| 31 | SEQ ID | SEQ ID NO: 44 | SEQ ID NO: 45 | SEQ ID NO: 28 | SEQ ID NO: 29 | SEQ ID NO: 35 |
| | NO: 25 | | | | | |
| SCT1. | ELSMH | IFDPKYGETMYAQKFOG | GGSPDYFYYYMDV | KASQDIDDDMN | EATTLVP | LQHDNFPYT |
| 32 | SEQ ID | SEQ ID NO: 46 | SEQ ID NO: 47 | SEQ ID NO: 28 | SEQ ID NO: 29 | SEQ ID NO: 35 |
| | NO: 25 | | | | | |
| SCT1. | ELSMH | IFDPRNGETIYAQKFQG | GGSPDYYYWYMDV | KASQDIDDDMN | EATTLVP | LQHDNFPYT |
| 33 | SEQ ID | SEQ ID NO: 48 | SEQ ID NO: 49 | SEQ ID NO: 28 | SEQ ID NO: 29 | SEQ ID NO: 35 |
| | NO: 25 | | | | | |
| SCT1. | ELSMH | IFDPRNGETIYAQKFQG | GGSPDYYFWYMDV | KASQDIDDDMN | EATTLVP | LQHDNFPYT |
| 34 | SEQ ID | SEQ ID NO: 48 | SEQ ID NO: 50 | SEQ ID NO: 28 | SEQ ID NO: 29 | SEQ ID NO: 35 |
| | NO: 25 | | | | | |
| SCT1. | SYDIN | WMNPNSGNTGSTQKFQG | GVLKGDYYYMDV | RASQSISSSYLS | GSSTRAT | QQDDHLPLT |
| 45 | SEQ ID | SEQ ID NO: 52 | SEQ ID NO: 53 | SEQ ID NO: 54 | SEQ ID NO: 55 | SEQ ID NO: 56 |
| | NO: 51 | | | | | |
| SCT1. | SYDIN | WVNPNSGNTGSTOKFQG | GVLKGDYYYMDV | RASQSISSTYLS | GSSTRAT | QQDDHLPLT |
| 46 | SEQ ID | SEQ ID NO: 57 | SEQ ID NO: 53 | SEQ ID NO: 58 | SEQ ID NO: 55 | SEQ ID NO: 56 |
| | NO: 51 | | | | | |
| SCT1. | SYDIN | WVNPNSGNTGSTQKFQG | GVLKGDYYYMDV | RASQSISSTYLS | GSSTRAT | QQDDHLPLT |
| 47 | SEQ ID | SEQ ID NO: 57 | SEQ ID NO: 53 | SEQ ID NO: 58 | SEQ ID NO: 55 | SEQ ID NO: 56 |
| | NO: 51 | | | | | |
| SCT1. | SYDIN | WVNPNSGNTGSTQKFQG | GVLKGDYYYMDV | RASQSISSTYLS | GSSTRAT | QQDDHLPLT |
| 48 | SEQ ID | SEQ ID NO: 57 | SEQ ID NO: 53 | SEQ ID NO: 58 | SEQ ID NO: 55 | SEQ ID NO: 56 |
| | NO: 51 | | | | | |
| kelixi- | GDYYWF | YIYGSGGGTNYNPSLNN | NILKYLHWLLY | GGDNVGRKSVQ | ADSERPS | QVWDSTADHWV |
| mab | SEQ ID | SEQ ID NO: 60 | SEQ ID NO: 61 | SEQ ID NO: 62 | SEQ ID NO: 63 | SEQ ID NO: 64 |
| | NO: 59 | | | | | |
| UB- | DYVIH | EIYPGSGSAYSNAKFKD | RGNGTGFAY | KAGQSVDYDGDSY | VASNLES | QQSYKDPLT |
| 421 | SEQ ID | SEQ ID NO: 66 | SEQ ID NO: 67 | MN | SEQ ID NO: 69 | SEQ ID NO: 70 |
| | NO: 65 | | | SEQ ID NO: 68 | | |

Binds to CD4 D2

| | | | | | | |
|---|---|---|---|---|---|---|
| ibalizumab | SYVIH | YINPYNDGTDYDEKFKG | EKDNYATGAWFAY | KSSQSLLYSTNQK | WASTRES | QQYYSYRT |
| | SEQ ID | SEQ ID NO: 72 | SEQ ID NO: 73 | NYLA | SEQ ID NO: 75 | SEQ ID NO: 76 |
| | NO: 71 | | | SEQ ID NO: 74 | | |
| tregalizumab | DCRMY | VISVKSENYGANYAESVRG | SYYRYDVGAWFAY | RASKSVSTSGYSY | LASILES | QHSRELPWT |
| | SEQ ID | SEQ ID NO: 78 | SEQ ID NO: 79 | IY | SEQ ID NO: 81 | SEQ ID NO: 82 |
| | NO: 77 | | | SEQ ID NO: 80 | | |
| SCT1. | ELSMH | GFDPEDGKTIYAPKFQG | GHNWNDGYYFYYYMDL | KSSQSLLHTDGKT | EVSNRFS | MQSLQLPLT |
| 1 | SEQ ID | | | YLY | | |
| | NO: 25 | SEQ ID NO: 83 | SEQ ID NO: 84 | SEQ ID NO: 85 | SEQ ID NO: 86 | SEQ ID NO: 87 |
| SCT1. | DYYMN | YISSSGNTIFYVDSVEG | EGYNNYNYSYYYFMDV | RASQSVSRKLA | AASTRAT | QQYNNWPYT |
| 4 | SEQ ID | SEQ ID NO: 89 | SEQ ID NO: 90 | SEQ ID NO: 91 | SEQ ID NO: 92 | SEQ ID NO: 93 |
| | NO: 88 | | | | | |
| SCT1. | SFGMH | IIWYDGTNKYYADSVKG | EIAVDGTDYYMDV | RASQGIRSGLG | AASTLQS | LQDYNYPYT |
| 5 | SEQ ID | SEQ ID NO: 95 | SEQ ID NO: 96 | SEQ ID NO: 97 | SEQ ID NO: 98 | SEQ ID NO: 99 |
| | NO: 94 | | | | | |
| SCT2. | DYYMN | YISSSGLTIFYVDSVKG | EGYSGFDDHYYYYTDV | RASQSVSSNLA | GASIRAT | QHYKNWPYT |
| 9 | SEQ ID | SEQ ID NO: 100 | SEQ ID NO: 101 | SEQ ID NO: 102 | SEQ ID | SEQ ID |
| | NO: 88 | | | | NO: 103 | NO: 104 |
| SCT2. | DYYMN | YISSSGSTIFYTDSVKG | EDYSNYEDYYYYYTGV | RASQSVSSKFA | NASTRAT | QQYNNWPYT |
| 10 | SEQ ID | SEQ ID NO: 105 | SEQ ID NO: 106 | SEQ ID NO: 107 | SEQ ID | SEQ ID NO: 93 |
| | NO: 88 | | | | NO: 108 | |
| SCT2. | DYYMN | YISSSGITIFYTDSVKG | EDYSNYEDHYYYYTGV | RASQSVSNKFA | NASTRAT | QHYNNWPYT |
| 11 | SEQ ID | SEQ ID NO: 109 | SEQ ID NO: 110 | SEQ ID NO: 111 | SEQ ID | SEQ ID |
| | NO: 88 | | | | NO: 108 | NO: 112 |
| SCT2. | DYYMN | YISSSGITIFYTDSVKG | EDYSNYEDHYYYYTGV | RASQSVSNKFA | GASTRAT | QHYNNWPYT |
| 11 | SEQ ID | SEQ ID NO: 109 | SEQ ID NO: 110 | SEQ ID NO: 111 | SEQ ID | SEQ ID |
| VL | NO: 88 | | | | NO: 113 | NO: 112 |
| N50G | | | | | | |
| SCT2. | DYYMN | YISSSGKTIFYTDSVKG | EDYSNYEDYYYYYTGV | RASQSVSSKFA | NASTRAT | HQYKNWPYT |
| 12 | SEQ ID | SEQ ID NO: 114 | SEQ ID NO: 106 | SEQ ID NO: 107 | SEQ ID | SEQ ID |
| | NO: 88 | | | | NO: 108 | NO: 115 |
| SCT2. | DYYMN | YISSSGKTIFYTDSVKG | EDYSNYEDYYYYYTGV | RASQSVSSKFA | GASTRAT | HOYKNWPYT |
| 12 | SEQ ID | SEQ ID NO: 114 | SEQ ID NO: 106 | SEQ ID NO: 107 | SEQ ID | SEQ ID |
| VL | NO: 88 | | | | NO: 113 | NO: 115 |
| N50G | | | | | | |
| SCT2. | DYYMN | YISISGQTIYYGDSVKG | EGYSNYGVKYYYYMDV | RASQSISSNLA | NASTRAT | QQYNAWTYT |
| 13 | SEQ ID | SEQ ID NO: 116 | SEQ ID NO: 117 | SEQ ID NO: 118 | SEQ ID | SEQ ID |
| | NO: 88 | | | | NO: 108 | NO: 119 |
| SCT2. | DHYMN | YISISGNTIYYTDSVKG | EGYSSSSRGDYSYYTDV | RASQSVSSNLA | GASTRAT | QQYKNWPYT |
| 14 | SEQ ID | SEQ ID NO: 121 | SEQ ID NO: 122 | SEQ ID NO: 102 | SEQ ID | SEQ ID |
| | NO: 120 | | | | NO: 113 | NO: 123 |
| SCT2. | DYYMN | YISISGRTIYYIDSVKG | EGYSSSSRGDYSYYTDV | RASQGVSSNLA | GASTRAT | QHYKNWPYT |
| 15 | SEQ ID | SEQ ID NO: 124 | SEQ ID NO: 122 | SEQ ID NO: 125 | SEQ ID | SEQ ID |
| | NO: 88 | | | | NO: 113 | NO: 104 |

Binds to CD4 D2 and/or D3

| | | | | | | |
|---|---|---|---|---|---|---|
| SCT1. | SYGMH | IIWFDGSNKYYADSLTG | EVALEGYYYYMDV | RASQDIRNGLG | TTSSLQT | LQDYNYPYT |
| 6 | SEQ ID | SEQ ID NO: 127 | SEQ ID NO: 128 | SEQ ID NO: 129 | SEQ ID | SEQ ID NO: 99 |
| | NO: 126 | | | | NO: 130 | |
| SCT1. | SYGMH | ILWFDGSNKFYADSVKG | ELALEGYYYYMDV | RASQDIRNGLG | TTSSLQS | LQDYNYPYT |
| 7 | SEQ ID | SEQ ID NO: 131 | SEQ ID NO: 132 | SEQ ID NO: 129 | SEQ ID | |
| | NO: 126 | | | | NO: 133 | SEQ ID NO: 99 |
| SCT1. | SYGMH | ILWFDGSNQFYADSVKG | EIALEGYYYYMDV | RSSQDIRNGLG | TTSSLQS | LQDYNYPYT |
| 8 | SEQ ID | SEQ ID NO: 134 | SEQ ID NO: 135 | SEQ ID NO: 136 | SEQ ID | SEQ ID NO: 99 |
| | NO: 126 | | | | NO: 133 | |
| SCT1. | SYGMH | IIWYDGSNKYYADSVKG | EVALEGYYYYMDV | RASQDIRNGLG | TTSNLQS | LQDYNYPYT |
| 9 | SEQ ID | SEQ ID NO: 137 | SEQ ID NO: 128 | SEQ ID NO: 129 | SEQ ID | SEQ ID NO: 99 |
| | NO: 126 | | | | NO: 138 | |
| SCT1. | SYGMH | IIWYDGNNKYYADSVKG | EVALEGYYYYMDV | RASQGIRNGLG | AASSLQS | LQDYNYPYT |
| 10 | SEQ ID | SEQ ID NO: 139 | SEQ ID NO: 128 | SEQ ID NO: 140 | SEQ ID | SEQ ID NO: 99 |
| | NO: 126 | | | | NO: 141 | |
| SCT1. | SYAMS | GISGSVGSTYYADSVKG | EGYDWSGMDV | RASQGIRNDLG | AASSLQS | LQDYNYPYT |
| 19 | SEQ ID | SEQ ID NO: 143 | SEQ ID NO: 144 | SEQ ID NO: 145 | SEQ ID | SEQ ID NO: 99 |
| | NO: 142 | | | | NO: 141 | |
| SCT2. | SYGMH | LIWFDGTTRFYADSVKG | EVAIQGRDYYIDV | RASQGIRNGLG | AASSLQS | LQDYNYPYT |
| 2 | SEQ ID | SEQ ID NO: 146 | SEQ ID NO: 147 | SEQ ID NO: 140 | SEQ ID | SEQ ID NO: 99 |
| | NO: 126 | | | | NO: 141 | |
| SCT2. | SYGIH | IIWFDGTNKFYADSVKG | EVAIQGYYYYMDV | RASQDIRNGLG | TTSNLQN | LQDYNYPYT |
| 3 | SEQ ID | SEQ ID NO: 149 | SEQ ID NO: 150 | SEQ ID NO: 129 | SEQ ID | SEQ ID NO: 99 |
| | NO: 148 | | | | NO: 151 | |

Binds to CD4 D3

| | | | | | | |
|---|---|---|---|---|---|---|
| OKT4 | NYGMN | WINTNTGEPTYAEEFKG | LGIYYDYGYYAMDY | RASESVDSYGNSF | LASNLES | QQNNEDPYT |
| | SEQ ID | SEQ ID NO: 153 | SEQ ID NO: 154 | MH | SEQ ID | SEQ ID |
| | NO: 152 | | | SEQ ID NO: 155 | NO: 156 | NO: 157 |
| SCT1. | TYAMS | GISGSGDNTYYADSVKG | EGYNWNYMDV | RASQGIRNDLG | EASSLQS | LQDYTYPYT |
| 20 | SEQ ID | SEQ ID NO: 159 | SEQ ID NO: 160 | SEQ ID NO: 145 | SEQ ID | SEQ ID |
| | NO: 158 | | | | NO: 161 | NO: 162 |
| SCT1. | TFAMS | GISGSGENTYYADSVKG | EGYNWNYMDV | RASHGIRNDLG | ETSSLQS | LQDYTYPYT |
| 21 | SEQ ID | SEQ ID NO: 164 | SEQ ID NO: 160 | SEQ ID NO: 165 | SEQ ID | SEQ ID |
| | NO: 163 | | | | NO: 166 | NO: 162 |
| SCT1. | TFAMS | GISGSGENTYYADSVKG | EGYNWNYMDV | RASHGIRNDLG | EASSLQS | LQDYTYPYT |
| 22 | SEQ ID | SEQ ID NO: 164 | SEQ ID NO: 160 | SEQ ID NO: 165 | SEQ ID | SEQ ID |
| | NO: 163 | | | | NO: 161 | NO: 162 |
| SCT1. | TYAMS | TISDNIGNTYYADSVKG | DNEDYYMDV | RASQGIRNDLG | AASTLQS | LQDYNYPRT |
| 23 | SEQ ID | SEQ ID NO: 167 | SEQ ID NO: 168 | SEQ ID NO: 145 | SEQ ID NO: 98 | SEQ ID |
| C36Y | NO: 158 | | | | | NO: 169 |
| SCT2. | TNAMS | GISGSNGNTYYADSVKG | ERDNWNGFDY | RASHVIRNDLG | AASSLQS | LQDYNYPYT |
| 1 | SEQ ID | SEQ ID NO: 171 | SEQ ID NO: 172 | SEQ ID NO: 173 | SEQ ID | SEQ ID NO: 99 |
| | NO: 170 | | | | NO: 141 | |
| SCT2. | ELSMH | IFDPRDGQIIYAEKFQG | GGNEDYYFYYMDV | KASQDIDDDVN | EATTLVP | LQHDHFPYT |
| 8 | SEQ ID | SEQ ID NO: 174 | SEQ ID NO: 175 | SEQ ID NO: 176 | SEQ ID NO: 29 | SEQ ID |
| | NO: 25 | | | | | NO: 177 |

TABLE A2
Illustrative CDRs for anti-CD4 binding domains (IMGT)

| | | | | | | |
|---|---|---|---|---|---|---|
| Ab | | | | | | |
| Name | VH - CDR1 | VH - CDR2 | VH - CDR3 | VL - CDR1 | VL - CDR2 | VL - CDR3 |

Binds to CD4 D1

| | | | | | | |
|---|---|---|---|---|---|---|
| 1 | GFSFSNYA | INX₁X₂GST | SRHYGGSYDPMDY | QDINKY | YTS | LQYDNPLX₇T |
| | SEQ ID | X₁ is A, D, E | SEQ ID NO: 180 | SEQ ID NO: 181 | SEQ ID | X₇ is F, Q, Y |
| | NO: 178 | X₂ is A, G, W, Y | | | NO: 182 | SEQ ID NO: 6 |
| | | SEQ ID NO: 179 | | | | |
| 2 | GFSFSNYA | INX₁X₂GST | SRHYGGSYDPMDY | QDINKY | YTS | LQYDNPLYT |
| | SEQ ID | X₁ is A, D, E | SEQ ID NO: 180 | SEQ ID NO: 181 | SEQ ID | SEQ ID NO: 9 |
| | NO: 178 | X₂ is A or G | | | NO: 182 | |
| | | SEQ ID NO: 183 | | | | |
| 3 | GFSFSNYA | INEX₂GST | SRHYGGSYDPMDY | QDINKY | YTS | LQYDNPLYT |
| | SEQ ID | X₂ is W or Y | SEQ ID NO: 180 | SEQ ID NO: 181 | SEQ ID | SEQ ID NO: 9 |
| | NO: 178 | SEQ ID NO: 184 | | | NO: 182 | |
| 4 | GFSFSNYA | INEX₂GST | SRHYGGSYDPMDY | QDINKY | YTS | LQYDNPLX₇T |
| | SEQ ID | X₂ is W or Y | SEQ ID NO: 180 | SEQ ID NO: 181 | SEQ ID | X₇ is F, Q, Y |
| | NO: 178 | SEQ ID NO: 184 | | | NO: 182 | SEQ ID NO: 6 |
| 5 | GFSFSNYA | INEX₂GST | SRHYGGSYDPMDY | QDINKY | YTS | LQYDNPLYT |
| | SEQ ID | X₂ is W or Y | SEQ ID NO: 180 | SEQ ID NO: 181 | SEQ ID | SEQ ID NO: 9 |
| | NO: 178 | SEQ ID NO: 184 | | | NO: 182 | |
| 6 | GFSFSNYA | INEGGST | SRHYGGSYDPMDY | QDINKY | YTS | LQYDNPLYT |
| | SEQ ID | SEQ ID NO: 185 | SEQ ID NO: 180 | SEQ ID NO: 181 | SEQ ID | SEQ ID NO: 9 |
| | NO: 178 | | | | NO: 182 | |
| 7 | GFSFSNYA | INEGGST | SRHYGGSYDPMDY | QDINKY | YTS | LQYDNPLYT |
| | SEQ ID | SEQ ID NO: 185 | SEQ ID NO: 180 | SEQ ID NO: 181 | SEQ ID | SEQ ID NO: 9 |
| | NO: 178 | | | | NO: 182 | |
| 8 | GFSFSNYA | INEWGST | SRHYGGSYDPMDY | QDINKY | YTS | LQYDNPLYT |
| | SEQ ID | SEQ ID NO: 186 | SEQ ID NO: 180 | SEQ ID NO: 181 | SEQ ID | SEQ ID NO: 9 |
| | NO: 178 | | | | NO: 182 | |
| 9 | GFSFSNYA | INEYGST | SRHYGGSYDPMDY | QDINKY | YTS | LQYDNPLYT |
| | SEQ ID | SEQ ID NO: 187 | SEQ ID NO: 180 | SEQ ID NO: 181 | SEQ ID | SEQ ID NO: 9 |
| | NO: 178 | | | | NO: 182 | |
| 10 | GFSFSNYA | INEWGST | SRHYGGSYDPMDY | QDINKY | YTS | LQYDNPLQT |
| | SEQ ID | SEQ ID NO: 186 | SEQ ID NO: 180 | SEQ ID NO: 181 | SEQ ID | SEQ ID NO: 17 |
| | NO: 178 | | | | NO: 182 | |
| 11 | GFSFSNYA | INEYGST | SRHYGGSYDPMDY | QDINKY | YTS | LQYDNPLFT |
| | SEQ ID | SEQ ID NO: 187 | SEQ ID NO: 180 | SEQ ID NO: 181 | SEQ ID | SEQ ID NO: 18 |
| | NO: 178 | | | | NO: 182 | |
| 12 | GFSFSNYA | INEGGST | SRHYGGSYDPMDY | QDINKY | YTS | LQYDNPLYT |
| | SEQ ID | SEQ ID NO: 185 | SEQ ID NO: 180 | SEQ ID NO: 181 | SEQ ID | SEQ ID NO: 9 |
| | NO: 178 | | | | NO: 182 | |
| 13 | GFSFSNYA | INDGGST | SRHYGGSYDPMDY | QDINKY | YTS | LQYDNPLYT |
| | SEQ ID | SEQ ID NO: 188 | SEQ ID NO: 180 | SEQ ID NO: 181 | SEQ ID | SEQ ID NO: 9 |
| | NO: 178 | | | | NO: 182 | |
| 14 | GFSFSNYA | INDGGST | SRHYGGSYDPMDY | QDINKY | YTS | LQYDNPLYT |
| | SEQ ID | SEQ ID NO: 188 | SEQ ID NO: 180 | SEQ ID NO: 181 | SEQ ID | SEQ ID NO: 9 |
| | NO: 178 | | | | NO: 182 | |
| 15 | GFSFSNYA | INAGGST | SRHYGGSYDPMDY | QDINKY | YTS | LQYDNPLYT |
| | SEQ ID | SEQ ID NO: 189 | SEQ ID NO: 180 | SEQ ID NO: 181 | SEQ ID | SEQ ID NO: 9 |
| | NO: 178 | | | | NO: 182 | |
| 16 | GFSFSNYA | INDAGST | SRHYGGSYDPMDY | QDINKY | YTS | LQYDNPLYT |
| | SEQ ID | SEQ ID NO: 190 | SEQ ID NO: 180 | SEQ ID NO: 181 | SEQ ID | SEQ ID NO: 9 |
| | NO: 178 | | | | NO: 182 | |
| 17 | GFSFSNYA | INDGGST | SRHYGGSYDPMDY | QDINKY | YTS | LQYDNPLYT |
| | SEQ ID | SEQ ID NO: 188 | SEQ ID NO: 180 | SEQ ID NO: 181 | SEQ ID | SEQ ID NO: 9 |
| | NO: 178 | | | | NO: 182 | |
| 18 | GFSFSNYA | INDGGST | SRHYGGSYDPMDY | QDINKY | YTS | LQYDNPLYT |
| | SEQ ID | SEQ ID NO: 188 | SEQ ID NO: 180 | SEQ ID NO: 181 | SEQ ID | SEQ ID NO: 9 |
| | NO: 178 | | | | NO: 182 | |
| 19 | GFSFSNYA | INDGGST | SRHYGGSYDPMDY | QDINKY | YTS | LQYDNPLYT |
| | SEQ ID | SEQ ID NO: 188 | SEQ ID NO: 180 | SEQ ID NO: 181 | SEQ ID | SEQ ID NO: 9 |
| | NO: 178 | | | | NO: 182 | |
| SCT1.17 | GFTEDDYG | INWNGDST | ARDGAIGGMDV | QDIRDD | SAS | LQDYNYPWT |
| | SEQ ID | SEQ ID NO: 192 | SEQ ID NO: 193 | SEQ ID NO: 194 | SEQ ID | SEQ ID NO: 24 |
| | NO: 191 | | | | NO: 195 | |
| SCT1.25 | GHTLTELS | FDPRGGET | ATGGDRDYYYYYMDV | QDIDDD | EAT | LQHDSFPYT |
| | SEQ ID | SEQ ID NO: 197 | SEQ ID NO: 198 | SEQ ID NO: 199 | SEQ ID | SEQ ID NO: 30 |
| | NO: 196 | | | | NO: 200 | |
| SCT1.26 | GFTLTKLS | FDPRDGER | ATGGDYDYYYYYMGV | QDIDDD | EAT | LQHDNFPYT |
| | SEQ ID | SEQ ID NO: 202 | SEQ ID NO: 203 | SEQ ID NO: 199 | SEQ ID | SEQ ID NO: 35 |
| | NO: 201 | | | | NO: 200 | |
| SCT1.27 | GHTVTELS | FHPRDDEI | ATGGNPDYYFYYMDV | QDIDDD | EAT | LQHDNFPLT |
| | SEQ ID | SEQ ID NO: 205 | SEQ ID NO: 206 | SEQ ID NO: 199 | SEQ ID | SEQ ID NO: 38 |
| | NO: 204 | | | | NO: 200 | |
| SCT1.28 | GHTLTELS | FHPRDGET | ATGGNKDYYFYYMDV | QDIDDD | EAT | LQHDNFPLT |
| | SEQ ID | SEQ ID NO: 207 | SEQ ID NO: 208 | SEQ ID NO: 199 | SEQ ID | SEQ ID NO: 38 |
| | NO: 196 | | | | NO: 200 | |
| SCT1.29 | GYTLTALS | FNPRDGET | ATGGDSDYYYYYMDV | QDIDDD | EAT | LQHDNFPYT |
| | SEQ ID | SEQ ID NO: 210 | SEQ ID NO: 211 | SEQ ID NO: 199 | SEQ ID | SEQ ID NO: 35 |
| | NO: 209 | | | | NO: 200 | |
| SCT1.30 | GHTLTELS | FNPRDDET | ATGGNKDYYFYYMDV | QDIDDD | EAT | LQHDNFPLT |
| | SEQ ID | SEQ ID NO: 212 | SEQ ID NO: 208 | SEQ ID NO: 199 | SEQ ID | SEQ ID NO: 38 |
| | NO: 196 | | | | NO: 200 | |
| SCT1.31 | GHTLTELS | FDPKYGET | ATGGNYEYFYYYMDV | QDIDDD | EAT | LQHDNFPYT |
| | SEQ ID | SEQ ID NO: 213 | SEQ ID NO: 214 | SEQ ID NO: 199 | SEQ ID | SEQ ID NO: 35 |
| | NO: 196 | | | | NO: 200 | |
| SCT1.32 | GHTLTELS | FDPKYGET | ATGGSPDYFYYYMDV | QDIDDD | EAT | LQHDNFPYT |
| | SEQ ID | SEQ ID NO: 213 | SEQ ID NO: 215 | SEQ ID NO: 199 | SEQ ID | SEQ ID NO: 35 |
| | NO: 196 | | | | NO: 200 | |
| SCT1.33 | GHTLTELS | FDPRNGET | ATGGSPDYYYWYMDV | QDIDDD | EAT | LQHDNFPYT |
| | SEQ ID | SEQ ID NO: 216 | SEQ ID NO: 217 | SEQ ID NO: 199 | SEQ ID | SEQ ID NO: 35 |
| | NO: 196 | | | | NO: 200 | |
| SCT1.34 | GHTLTELS | FDPRNGET | ATGGSPDYYFWYMDV | QDIDDD | EAT | LQHDNFPYT |
| | SEQ ID | SEQ ID NO: 216 | SEQ ID NO: 218 | SEQ ID NO: 199 | SEQ ID | SEQ ID NO: 35 |
| | NO: 196 | | | | NO: 200 | |
| SCT1.45 | GYTFTSYD | MNPNSGNT | ARGVLKGDYYYMDV | QSISSSY | GSS | QQDDHLPLT |
| | SEQ ID | SEQ ID NO: 220 | SEQ ID NO: 221 | SEQ ID NO: 222 | SEQ ID | SEQ ID NO: 56 |
| | NO: 219 | | | | NO: 223 | |
| SCT1.46 | GYTFTSYD | VNPNSGNT | ARGVLKGDYYYMDV | QSISSTY | GSS | QQDDHLPLT |
| | SEQ ID | SEQ ID NO: 224 | SEQ ID NO: 221 | SEQ ID NO: 225 | SEQ ID | SEQ ID NO: 56 |
| | NO: 219 | | | | NO: 223 | |
| SCT1.47 | GYTFTSYD | VNPNSGNT | ARGVLKGDYYYMDV | QSISSTY | GSS | QQDDHLPLT |
| | SEQ ID | SEQ ID NO: 224 | SEQ ID NO: 221 | SEQ ID NO: 225 | SEQ ID | SEQ ID NO: 56 |
| | NO: 219 | | | | NO: 223 | |
| SCT1.48 | GYTFTSYD | VNPNSGNT | ARGVLKGDYYYMDV | QSISSTY | GSS | QQDDHLPLT |
| | SEQ ID | SEQ ID NO: 224 | SEQ ID NO: 221 | SEQ ID NO: 225 | SEQ ID | SEQ ID NO: 56 |
| | NO: 219 | | | | NO: 223 | |
| keliximab | GGSISGDYY | IYGSGGGT | ASNILKYLHWLLY | NVGRKS | ADS | QVWDSTADHWV |
| | SEQ ID | SEQ ID NO: 227 | SEQ ID NO: 228 | SEQ ID NO: 229 | SEQ ID | SEQ ID NO: 64 |
| | NO: 226 | | | | NO: 230 | |
| UB-421 | GYTFTDYV | IYPGSGSA | ARRGNGTGFAY | QSVDYDGDSY | VAS | QQSYKDPLT |
| | SEQ ID | SEQ ID NO: 232 | SEQ ID NO: 233 | SEQ ID NO: 234 | SEQ ID | SEQ ID NO: 70 |
| | NO: 231 | | | | NO: 235 | |

Binds to CD4 D2

| | | | | | | |
|---|---|---|---|---|---|---|
| ibalizumab | GYTFTSYV | INPYNDGT | AREKDNYATGAWFAY | QSLLYSTNQKNY | WAS | QQYYSYRT |
| | SEQ ID | SEQ ID NO: 237 | SEQ ID NO: 238 | SEQ ID NO: 239 | SEQ ID | SEQ ID NO: 76 |
| | NO: 236 | | | | NO: 240 | |
| tregalizumab | GFSFSDCR | ISVKSENYGA | SASYYRYDVGAWFAY | KSVSTSGYSY | LAS | QHSRELPWT |
| | SEQ ID | SEQ ID NO: 242 | SEQ ID NO: 243 | SEQ ID NO: 244 | SEQ ID | SEQ ID NO: 82 |
| | NO: 241 | | | | NO: 245 | |
| SCT1.1 | GYTLTELS | FDPEDGKT | ATGHNWNDGYYFYYYM | QSLLHTDGKTY | EVS | MQSLQLPLT |
| | SEQ ID | SEQ ID NO: 247 | DL | SEQ ID NO: 249 | SEQ ID | SEQ ID NO: 87 |
| | NO: 246 | | SEQ ID NO: 248 | | NO: 250 | |
| SCT1.4 | GFTFSDYY | ISSSGNTI | AREGYNNYNYSYYYFM | QSVSRK | AAS | QQYNNWPYT |
| | SEQ ID | SEQ ID NO: 252 | DV | SEQ ID NO: 254 | SEQ ID | SEQ ID NO: 93 |
| | NO: 251 | | SEQ ID NO: 253 | | NO: 255 | |
| SCT1.5 | GFTVSSFG | IWYDGTNK | AREIAVDGTDYYMDV | QGIRSG | AAS | LQDYNYPYT |
| | SEQ ID | SEQ ID NO: 257 | SEQ ID NO: 258 | SEQ ID NO: 259 | SEQ ID | SEQ ID NO: 99 |
| | NO: 256 | | | | NO: 255 | |
| SCT2.9 | GFTFSDYY | ISSSGLTI | AREGYSGFDDHYYYYT | QSVSSN | GAS | QHYKNWPYT |
| | SEQ ID | SEQ ID NO: 260 | DV | SEQ ID NO: 262 | SEQ ID | SEQ ID |
| | NO: 251 | | SEQ ID NO: 261 | | NO: 263 | NO: 104 |
| SCT2.10 | GFTFSDYY | ISSSGSTI | AREDYSNYEDYYYYYT | QSVSSK | NAS | QQYNNWPYT |
| | SEQ ID | SEQ ID NO: 264 | GV | SEQ ID NO: 266 | SEQ ID | SEQ ID NO: 93 |
| | NO: 251 | | SEQ ID NO: 265 | | NO: 267 | |
| SCT2.11 | GFTFSDYY | ISSSGITI | AREDYSNYEDHYYYYT | QSVSNK | NAS | QHYNNWPYT |
| | SEQ ID | SEQ ID NO: 268 | GV | SEQ ID NO: 270 | SEQ ID | SEQ ID |
| | NO: 251 | | SEQ ID NO: 269 | | NO: 267 | NO: 112 |
| SCT2.11 | GFTFSDYY | ISSSGITI | AREDYSNYEDHYYYYT | QSVSNK | GAS | QHYNNWPYT |
| VL N50G | SEQ ID | SEQ ID NO: 268 | GV | SEQ ID NO: 270 | SEQ ID | SEQ ID |
| | NO: 251 | | SEQ ID NO: 269 | | NO: 263 | NO: 112 |
| SCT2.12 | GFTFSDYY | ISSSGKTI | AREDYSNYEDYYYYYT | QSVSSK | NAS | HQYKNWPYT |
| | SEQ ID | SEQ ID NO: 271 | GV | SEQ ID NO: 266 | SEQ ID | SEQ ID |
| | NO: 251 | | SEQ ID NO: 265 | | NO: 267 | NO: 115 |
| SCT2.12 | GFTFSDYY | ISSSGKTI | AREDYSNYEDYYYYYT | QSVSSK | GAS | HQYKNWPYT |
| VL N50G | SEQ ID | SEQ ID NO: 271 | GV | SEQ ID NO: 266 | SEQ ID | SEQ ID |
| | NO: 251 | | SEQ ID NO: 265 | | NO: 263 | NO: 115 |
| SCT2.13 | GFTFSDYY | ISISGQTI | AREGYSNYGVKYYYYM | QSISSN | NAS | QQYNAWTYT |
| | SEQ ID | SEQ ID NO: 272 | DV | SEQ ID NO: 274 | SEQ ID | SEQ ID |
| | NO: 251 | | SEQ ID NO: 273 | | NO: 267 | NO: 119 |
| SCT2.14 | GFTFSDHY | ISISGNTI | VREGYSSSSRGDYSYY | QSVSSN | GAS | QQYKNWPYT |
| | SEQ ID | SEQ ID NO: 276 | TDV | SEQ ID NO: 262 | SEQ ID | SEQ ID |
| | NO: 275 | | SEQ ID NO: 277 | | NO: 263 | NO: 123 |
| SCT2.15 | GFTFSDYY | ISISGRTI | VREGYSSSSRGDYSYY | QGVSSN | GAS | QHYKNWPYT |
| | SEQ ID | SEQ ID NO: 278 | TDV | SEQ ID NO: 279 | SEQ ID | SEQ ID |
| | NO: 251 | | SEQ ID NO: 277 | | NO: 263 | NO: 104 |

Binds to CD4 D2 and/or D3

| | | | | | | |
|---|---|---|---|---|---|---|
| SCT1.6 | GFTVSSYG | IWFDGSNK | AREVALEGYYYYMDV | QDIRNG | TTS | LQDYNYPYT |
| | SEQ ID | SEQ ID NO: 281 | SEQ ID NO: 282 | SEQ ID NO: 283 | SEQ ID | SEQ ID NO: 99 |
| | NO: 280 | | | | NO: 284 | |
| SCT1.7 | GFTVSSYG | LWFDGSNK | ARELALEGYYYYMDV | QDIRNG | TTS | LQDYNYPYT |
| | SEQ ID | SEQ ID NO: 285 | SEQ ID NO: 286 | SEQ ID NO: 283 | SEQ ID | SEQ ID NO: 99 |
| | NO: 280 | | | | NO: 284 | |
| SCT1.8 | GFTVSSYG | LWFDGSNQ | AREIALEGYYYYMDV | QDIRNG | TTS | LQDYNYPYT |
| | SEQ ID | SEQ ID NO: 287 | SEQ ID NO: 288 | SEQ ID NO: 283 | SEQ ID | SEQ ID NO: 99 |
| | NO: 280 | | | | NO: 284 | |
| SCT1.9 | GFTFSSYG | IWYDGSNK | AREVALEGYYYYMDV | QDIRNG | TTS | LQDYNYPYT |
| | SEQ ID | | | | SEQ ID | |
| | NO: 289 | SEQ ID NO: 290 | SEQ ID NO: 282 | SEQ ID NO: 283 | NO: 284 | SEQ ID NO: 99 |
| SCT1.10 | GFTFSSYG | IWYDGNNK | TREVALEGYYYYMDV | QGIRNG | AAS | LQDYNYPYT |
| | SEQ ID | SEQ ID NO: 291 | SEQ ID NO: 292 | SEQ ID NO: 293 | SEQ ID | SEQ ID NO: 99 |
| | NO: 289 | | | | NO: 255 | |
| SCT1.19 | GFTFSSYA | ISGSVGST | AKEGYDWSGMDV | QGIRND | AAS | LQDYNYPYT |
| | SEQ ID | SEQ ID NO: 295 | SEQ ID NO: 296 | SEQ ID NO: 297 | SEQ ID | SEQ ID NO: 99 |
| | NO: 294 | | | | NO: 255 | |
| SCT2.2 | GFTFSSYG | IWFDGTTR | AREVAIQGRDYYIDV | QGIRNG | AAS | LQDYNYPYT |
| | SEQ ID | SEQ ID NO: 298 | SEQ ID NO: 299 | SEQ ID NO: 293 | SEQ ID | SEQ ID NO: 99 |
| | NO: 289 | | | | NO: 255 | |
| SCT2.3 | GFTFSSYG | IWFDGTNK | AREVAIQGYYYYMDV | QDIRNG | TTS | LQDYNYPYT |
| | SEQ ID | SEQ ID NO: 300 | SEQ ID NO: 301 | SEQ ID NO: 283 | SEQ ID | SEQ ID NO: 99 |
| | NO: 289 | | | | NO: 284 | |

Binds to CD4 D3

| | | | | | | |
|---|---|---|---|---|---|---|
| OKT4 | GYTFTNYG | INTNTGEP | ARLGIYYDYGYYAMDY | ESVDSYGNSF | LAS | QQNNEDPYT |
| | SEQ ID | SEQ ID NO: 303 | SEQ ID NO: 304 | SEQ ID NO: 305 | SEQ ID | SEQ ID |
| | NO: 302 | | | | NO: 245 | NO: 157 |
| SCT1.20 | EIIFSTYA | ISGSGDNT | VKEGYNWNYMDV | QGIRND | EAS | LQDYTYPYT |
| | SEQ ID | SEQ ID NO: 307 | SEQ ID NO: 1083 | SEQ ID NO: 297 | SEQ ID | SEQ ID |
| | NO: 306 | | | | NO: 308 | NO: 162 |
| SCT1.21 | GLTFSTFA | ISGSGENT | AKEGYNWNYMDV | HGIRND | ETS | LQDYTYPYT |
| | SEQ ID | SEQ ID NO: 310 | SEQ ID NO: 311 | SEQ ID NO: 312 | SEQ ID | SEQ ID |
| | NO: 309 | | | | NO: 313 | NO: 162 |
| SCT1.22 | GLTESTFA | ISGSGENT | AKEGYNWNYMDV | HGIRND | EAS | LQDYTYPYT |
| | SEQ ID | SEQ ID NO: 310 | SEQ ID NO: 311 | SEQ ID NO: 312 | SEQ ID | SEQ ID |
| | NO: 309 | | | | NO: 308 | NO: 162 |
| SCT1.23 | GFTFSTYA | ISDNIGNT | TKDNEDYYMDV | QGIRND | AAS | LQDYNYPRT |
| C36Y | SEQ ID | SEQ ID NO: 315 | SEQ ID NO: 316 | SEQ ID NO: 297 | SEQ ID | SEQ ID |
| | NO: 314 | | | | NO: 255 | NO: 169 |
| SCT2.1 | GFTESTNA | ISGSNGNT | AKERDNWNGFDY | HVIRND | AAS | LQDYNYPYT |
| | SEQ ID | SEQ ID NO: 318 | SEQ ID NO: 319 | SEQ ID NO: 320 | SEQ ID | SEQ ID NO: 99 |
| | NO: 317 | | | | NO: 255 | |
| SCT2.8 | GHTLTELS | FDPRDGQI | ATGGNEDYYFYYMDV | QDIDDD | EAT | LQHDHFPYT |
| | SEQ ID | SEQ ID NO: 321 | SEQ ID NO: 322 | SEQ ID NO: 199 | SEQ ID | SEQ ID |
| | NO: 196 | | | | NO: 200 | NO: 177 |

TABLE A3
Illustrative CDRs for anti-CD4 binding domains (Chothia)

| Ab | VH - | | | | | |
|---|---|---|---|---|---|---|
| Name | CDR1 | VH - CDR2 | VH - CDR3 | VL - CDR1 | VL - CDR2 | VL - CDR3 |
| 1 | GFSFSNY | X₁X₂G | YGGSYDPMD | SQDINKY | YTS | YDNPLX₇ |
| | SEQ ID | X₁ is A, D, E | SEQ ID NO: 325 | SEQ ID NO: 326 | SEQ ID | X₇ is F, Q, Y |
| | NO: 323 | X₂ is A, G, W, Y | | | NO: 182 | SEQ ID NO: 327 |
| | | SEQ ID NO: 324 | | | | |
| 2 | GFSFSNY | X₁X₂G | YGGSYDPMD | SQDINKY | YTS | YDNPLY |
| | SEQ ID | X₁ is A, D, E | SEQ ID NO: 325 | SEQ ID NO: 326 | SEQ ID | SEQ ID NO: 329 |
| | NO: 323 | X₂ is A or G | | | NO: 182 | |
| | | SEQ ID NO: 328 | | | | |
| 4 | GFSFSNY | EX₂G | YGGSYDPMD | SQDINKY | YTS | YDNPLX₇ |
| | SEQ ID | X₂ is W or Y | SEQ ID NO: 325 | SEQ ID NO: 326 | SEQ ID | X₇ is F, Q, Y |
| | NO: 323 | SEQ ID NO: 330 | | | NO: 182 | SEQ ID NO: 327 |
| 5 | GFSFSNY | EX₂G | YGGSYDPMD | SQDINKY | YTS | YDNPLY |
| | SEQ ID | X₂ is W or Y | SEQ ID NO: 325 | SEQ ID NO: 326 | SEQ ID | SEQ ID NO: 329 |
| | NO: 323 | SEQ ID NO: 330 | | | NO: 182 | |
| 6 | GFSFSNY | EGG | YGGSYDPMD | SQDINKY | YTS | YDNPLY |
| | SEQ ID | SEQ ID NO: 331 | SEQ ID NO: 325 | SEQ ID NO: 326 | SEQ ID | SEQ ID NO: 329 |
| | NO: 323 | | | | NO: 182 | |
| 7 | GFSFSNY | EGG | YGGSYDPMD | SQDINKY | YTS | YDNPLY |
| | SEQ ID | SEQ ID NO: 331 | SEQ ID NO: 325 | SEQ ID NO: 326 | SEQ ID | SEQ ID NO: 329 |
| | NO: 323 | | | | NO: 182 | |
| 8 | GFSFSNY | EWG | YGGSYDPMD | SQDINKY | YTS | YDNPLY |
| | SEQ ID | SEQ ID NO: 332 | SEQ ID NO: 325 | SEQ ID NO: 326 | SEQ ID | SEQ ID NO: 329 |
| | NO: 323 | | | | NO: 182 | |
| 9 | GFSESNY | EYG | YGGSYDPMD | SQDINKY | YTS | YDNPLY |
| | SEQ ID | SEQ ID NO: 333 | SEQ ID NO: 325 | SEQ ID NO: 326 | SEQ ID | SEQ ID NO: 329 |
| | NO: 323 | | | | NO: 182 | |
| 10 | GFSFSNY | EWG | YGGSYDPMD | SQDINKY | YTS | YDNPLQ |
| | SEQ ID | SEQ ID NO: 332 | SEQ ID NO: 325 | SEQ ID NO: 326 | SEQ ID | SEQ ID NO: 334 |
| | NO: 323 | | | | NO: 182 | |
| 11 | GFSFSNY | EYG | YGGSYDPMD | SQDINKY | YTS | YDNPLE |
| | SEQ ID | SEQ ID NO: 333 | SEQ ID NO: 325 | SEQ ID NO: 326 | SEQ ID | SEQ ID NO: 335 |
| | NO: 323 | | | | NO: 182 | |
| 12 | GFSFSNY | EGG | YGGSYDPMD | SQDINKY | YTS | YDNPLY |
| | SEQ ID | SEQ ID NO: 331 | SEQ ID NO: 325 | SEQ ID NO: 326 | SEQ ID | SEQ ID NO: 329 |
| | NO: 323 | | | | NO: 182 | |
| 13 | GFSFSNY | DGG | YGGSYDPMD | SQDINKY | YTS | YDNPLY |
| | SEQ ID | SEQ ID NO: 336 | SEQ ID NO: 325 | SEQ ID NO: 326 | SEQ ID | SEQ ID NO: 329 |
| | NO: 323 | | | | NO: 182 | |
| 14 | GFSFSNY | DGG | YGGSYDPMD | SQDINKY | YTS | YDNPLY |
| | SEQ ID | SEQ ID NO: 336 | SEQ ID NO: 325 | SEQ ID NO: 326 | SEQ ID | SEQ ID NO: 329 |
| | NO: 323 | | | | NO: 182 | |
| 15 | GFSFSNY | AGG | YGGSYDPMD | SQDINKY | YTS | YDNPLY |
| | SEQ ID | SEQ ID NO: 337 | SEQ ID NO: 325 | SEQ ID NO: 326 | SEQ ID | SEQ ID NO: 329 |
| | NO: 323 | | | | NO: 182 | |
| 16 | GFSFSNY | DAG | YGGSYDPMD | SQDINKY | YTS | YDNPLY |
| | SEQ ID | SEQ ID NO: 338 | SEQ ID NO: 325 | SEQ ID NO: 326 | SEQ ID | SEQ ID NO: 329 |
| | NO: 323 | | | | NO: 182 | |
| 17 | GFSFSNY | DGG | YGGSYDPMD | SQDINKY | YTS | YDNPLY |
| | SEQ ID | SEQ ID NO: 336 | SEQ ID NO: 325 | SEQ ID NO: 326 | SEQ ID | SEQ ID NO: 329 |
| | NO: 323 | | | | NO: 182 | |
| 18 | GFSFSNY | DGG | YGGSYDPMD | SQDINKY | YTS | YDNPLY |
| | SEQ ID | SEQ ID NO: 336 | SEQ ID NO: 325 | SEQ ID NO: 326 | SEQ ID | SEQ ID NO: 329 |
| | NO: 323 | | | | NO: 182 | |
| 19 | GFSFSNY | DGG | YGGSYDPMD | SQDINKY | YTS | YDNPLY |
| | SEQ ID | SEQ ID NO: 336 | SEQ ID NO: 325 | SEQ ID NO: 326 | SEQ ID | SEQ ID NO: 329 |
| | NO: 323 | | | | NO: 182 | |
| SCT1.17 | GFTEDDY | WNGD | GAIGGMD | SQDIRDD | SAS | DYNYPW |
| | SEQ ID | SEQ ID NO: 340 | SEQ ID NO: 341 | SEQ ID NO: 342 | SEQ ID | SEQ ID |
| | NO: 339 | | | | NO: 195 | NO: 1131 |
| SCT1.25 | GHTLTEL | PRGG | GDRDYYYYYMD | SQDIDDD | EAT | HDSFPY |
| | SEQ ID | SEQ ID NO: 345 | SEQ ID NO: 346 | SEQ ID NO: 347 | SEQ ID | SEQ ID NO: 348 |
| | NO: 344 | | | | NO: 200 | |
| SCT1.26 | GFTLTKI | PRDG | GDYDYYYYYMG | SQDIDDD | EAT | HDNFPY |
| | SEQ ID | SEQ ID NO: 350 | SEQ ID NO: 351 | SEQ ID NO: 347 | SEQ ID | SEQ ID NO: 352 |
| | NO: 349 | | | | NO: 200 | |
| SCT1.27 | GHTVTEL | PRDD | GNPDYYFYYMD | SQDIDDD | EAT | HDNFPL |
| | SEQ ID | SEQ ID NO: 354 | SEQ ID NO: 355 | SEQ ID NO: 347 | SEQ ID | SEQ ID NO: 356 |
| | NO: 353 | | | | NO: 200 | |
| SCT1.28 | GHTLTEL | PRDG | GNKDYYFYYMD | SQDIDDD | EAT | HDNFPL |
| | SEQ ID | SEQ ID NO: 350 | SEQ ID NO: 357 | SEQ ID NO: 347 | SEQ ID | SEQ ID NO: 356 |
| | NO: 344 | | | | NO: 200 | |
| SCT1.29 | GYTLTAL | PRDG | GDSDYYYYYMD | SQDIDDD | EAT | HDNFPY |
| | SEQ ID | SEQ ID NO: 350 | SEQ ID NO: 359 | SEQ ID NO: 347 | SEQ ID | SEQ ID NO: 352 |
| | NO: 358 | | | | NO: 200 | |
| SCT1.30 | GHTLTEL | PRDD | GNKDYYFYYMD | SQDIDDD | EAT | HDNFPL |
| | SEQ ID | SEQ ID NO: 354 | SEQ ID NO: 357 | SEQ ID NO: 347 | SEQ ID | SEQ ID NO: 356 |
| | NO: 343 | | | | NO: 200 | |
| SCT1.31 | GHTLTEL | PKYG | GNYEYFYYYMD | SQDIDDD | EAT | HDNFPY |
| | SEQ ID | SEQ ID NO: 360 | SEQ ID NO: 361 | SEQ ID NO: 347 | SEQ ID | SEQ ID NO: 352 |
| | NO: 343 | | | | NO: 200 | |
| SCT1.32 | GHTLTEL | PKYG | GSPDYFYYYMD | SQDIDDD | EAT | HDNFPY |
| | SEQ ID | SEQ ID NO: 360 | SEQ ID NO: 362 | SEQ ID NO: 347 | SEQ ID | SEQ ID NO: 352 |
| | NO: 343 | | | | NO: 200 | |
| SCT1.33 | GHTLTEL | PRNG | GSPDYYYWYMD | SQDIDDD | EAT | HDNFPY |
| | SEQ ID | SEQ ID NO: 363 | SEQ ID NO: 364 | SEQ ID NO: 347 | SEQ ID | SEQ ID NO: 352 |
| | NO: 343 | | | | NO: 200 | |
| SCT1.34 | GHTLTEL | PRNG | GSPDYYFWYMD | SQDIDDD | EAT | HDNFPY |
| | SEQ ID | SEQ ID NO: 363 | SEQ ID NO: 365 | SEQ ID NO: 347 | SEQ ID | SEQ ID NO: 352 |
| | NO: 343 | | | | NO: 200 | |
| SCT1.45 | GYTFTSY | PNSG | VLKGDYYYMD | SQSISSSY | GSS | DDHLPL |
| | SEQ ID | SEQ ID NO: 367 | SEQ ID NO: 368 | SEQ ID | SEQ ID | SEQ ID NO: 369 |
| | NO: 366 | | | NO: 1084 | NO: 223 | |
| SCT1.46 | GYTFTSY | PNSG | VLKGDYYYMD | SQSISSTY | GSS | DDHLPL |
| | SEQ ID | SEQ ID NO: 367 | SEQ ID NO: 368 | SEQ ID NO: 370 | SEQ ID | SEQ ID NO: 369 |
| | NO: 366 | | | | NO: 223 | |
| SCT1.47 | GYTFTSY | PNSG | VLKGDYYYMD | SQSISSTY | GSS | DDHLPL |
| | SEQ ID | SEQ ID NO: 367 | SEQ ID NO: 368 | SEQ ID NO: 370 | SEQ ID | SEQ ID NO: 369 |
| | NO: 366 | | | | NO: 223 | |
| SCT1.48 | GYTFTSY | PNSG | VLKGDYYYMD | SQSISSTY | GSS | DDHLPL |
| | SEQ ID | SEQ ID NO: 367 | SEQ ID NO: 368 | SEQ ID NO: 370 | SEQ ID | SEQ ID NO: 369 |
| | NO: 366 | | | | NO: 223 | |
| keliximab | GGSISGDY | GSGG | ILKYLHWLL | GDNVGRKS | ADS | WDSTADHW |
| | SEQ ID | SEQ ID NO: 372 | SEQ ID NO: 373 | SEQ ID NO: 374 | SEQ ID | SEQ ID NO: 375 |
| | NO: 371 | | | | NO: 230 | |
| UB-421 | GYTFTDY | PGSG | GNGTGFA | GQSVDYDGDSY | VAS | SYKDPL |
| | SEQ ID | SEQ ID NO: 377 | SEQ ID NO: 378 | SEQ ID NO: 379 | SEQ ID | SEQ ID NO: 380 |
| | NO: 376 | | | | NO: 235 | |

Binds to CD4 D2

| | | | | | | |
|---|---|---|---|---|---|---|
| ibalizumab | GYTFTSY | PYND | KDNYATGAWFA | SQSLLYSTNQKNY | WAS | YYSYR |
| | SEQ ID | SEQ ID NO: 381 | SEQ ID NO: 382 | SEQ ID NO: 383 | SEQ ID | SEQ ID NO: 384 |
| | NO: 366 | | | | NO: 240 | |
| tregalizumab | GFSFSDC | VKSENY | YYRYDVGAWFA | SKSVSTSGYSY | LAS | SRELPW |
| | SEQ ID | SEQ ID NO: 386 | SEQ ID NO: 387 | SEQ ID NO: 388 | SEQ ID | SEQ ID NO: 389 |
| | NO: 385 | | | | NO: 245 | |
| SCT1.1 | GYTLTEL | PEDG | HNWNDGYYFYYYMD | SQSLLHTDGKTY | EVS | SLQLPL |
| | SEQ ID | SEQ ID NO: 391 | SEQ ID NO: 392 | SEQ ID NO: 393 | SEQ ID | SEQ ID NO: 394 |
| | NO: 390 | | | | NO: 250 | |
| SCT1.4 | GFTFSDY | SSGN | GYNNYNYSYYYFMD | SQSVSRK | AAS | YNNWPY |
| | SEQ ID | SEQ ID NO: 396 | SEQ ID NO: 397 | SEQ ID NO: 398 | SEQ ID | SEQ ID NO: 399 |
| | NO: 395 | | | | NO: 255 | |
| SCT1.5 | GFTVSSF | YDGT | IAVDGTDYYMD | SQGIRSG | AAS | DYNYPY |
| | SEQ ID | SEQ ID NO: 401 | SEQ ID NO: 402 | SEQ ID NO: 403 | SEQ ID | SEQ ID NO: 404 |
| | NO: 400 | | | | NO: 255 | |
| SCT2.9 | GFTFSDY | SSGL | GYSGFDDHYYYYTD | SQSVSSN | GAS | YKNWPY |
| | SEQ ID | SEQ ID NO: 405 | SEQ ID NO: 406 | SEQ ID NO: 407 | SEQ ID | SEQ ID NO: 408 |
| | NO: 395 | | | | NO: 263 | |
| SCT2.10 | GFTFSDY | SSGS | DYSNYEDYYYYYTG | SQSVSSK | NAS | YNNWPY |
| | SEQ ID | SEQ ID NO: 409 | SEQ ID NO: 410 | SEQ ID NO: 411 | SEQ ID | SEQ ID NO: 399 |
| | NO: 395 | | | | NO: 267 | |
| SCT2.11 | GFTFSDY | SSGI | DYSNYEDHYYYYTG | SQSVSNK | NAS | YNNWPY |
| | SEQ ID | SEQ ID NO: 412 | SEQ ID NO: 413 | SEQ ID NO: 414 | SEQ ID | SEQ ID NO: 399 |
| | NO: 395 | | | | NO: 267 | |
| SCT2.11 | GFTFSDY | SSGI | DYSNYEDHYYYYTG | SQSVSNK | GAS | YNNWPY |
| VL N50G | SEQ ID | SEQ ID NO: 412 | SEQ ID NO: 413 | SEQ ID NO: 414 | SEQ ID | SEQ ID NO: 399 |
| | NO: 395 | | | | NO: 263 | |
| SCT2.12 | GFTFSDY | SSGK | DYSNYEDYYYYYTG | SQSVSSK | NAS | YKNWPY |
| | SEQ ID | SEQ ID NO: 415 | SEQ ID NO: 410 | SEQ ID NO: 411 | SEQ ID | SEQ ID NO: 408 |
| | NO: 395 | | | | NO: 267 | |
| SCT2.12 | GFTFSDY | SSGK | DYSNYEDYYYYYTG | SQSVSSK | GAS | YKNWPY |
| VL N50G | SEQ ID | SEQ ID NO: 415 | SEQ ID NO: 410 | SEQ ID NO: 411 | SEQ ID | SEQ ID NO: 408 |
| | NO: 395 | | | | NO: 263 | |
| SCT2.13 | GFTFSDY | ISGQ | GYSNYGVKYYYYMD | SQSISSN | NAS | YNAWTY |
| | SEQ ID | SEQ ID NO: 416 | SEQ ID NO: 417 | SEQ ID NO: 418 | SEQ ID | SEQ ID NO: 419 |
| | NO: 395 | | | | NO: 267 | |
| SCT2.14 | GFTFSDH | ISGN | GYSSSSRGDYSYYTD | SQSVSSN | GAS | YKNWPY |
| | SEQ ID | SEQ ID NO: 421 | SEQ ID NO: 422 | SEQ ID NO: 407 | SEQ ID | SEQ ID NO: 408 |
| | NO: 420 | | | | NO: 263 | |
| SCT2.15 | GFTFSDY | ISGR | GYSSSSRGDYSYYTD | SQGVSSN | GAS | YKNWPY |
| | SEQ ID | SEQ ID NO: 423 | SEQ ID NO: 422 | SEQ ID NO: 424 | SEQ ID | SEQ ID NO: 408 |
| | NO: 395 | | | | NO: 263 | |

Binds to CD4 D2 and/or D3

| | | | | | | |
|---|---|---|---|---|---|---|
| SCT1.6 | GFTVSSY | FDGS | VALEGYYYYMD | SQDIRNG | TTS | DYNYPY |
| | SEQ ID | SEQ ID NO: 426 | SEQ ID NO: 427 | SEQ ID NO: 428 | SEQ ID | SEQ ID NO: 404 |
| | NO: 425 | | | | NO: 284 | |
| SCT1.7 | GFTVSSY | FDGS | LALEGYYYYMD | SQDIRNG | TTS | DYNYPY |
| | SEQ ID | SEQ ID NO: 426 | SEQ ID NO: 429 | SEQ ID NO: 428 | SEQ ID | SEQ ID NO: 404 |
| | NO: 425 | | | | NO: 284 | |
| SCT1.8 | GFTVSSY | FDGS | IALEGYYYYMD | SQDIRNG | TTS | DYNYPY |
| | SEQ ID | SEQ ID NO: 426 | SEQ ID NO: 430 | SEQ ID NO: 428 | SEQ ID | SEQ ID NO: 404 |
| | NO: 425 | | | | NO: 284 | |
| SCT1.9 | GFTFSSY | YDGS | VALEGYYYYMD | SQDIRNG | TTS | DYNYPY |
| | SEQ ID | SEQ ID NO: 432 | SEQ ID NO: 427 | SEQ ID NO: 428 | SEQ ID | SEQ ID NO: 404 |
| | NO: 431 | | | | NO: 284 | |
| SCT1.10 | GFTFSSY | YDGN | VALEGYYYYMD | SQGIRNG | AAS | DYNYPY |
| | SEQ ID | SEQ ID NO: 433 | SEQ ID NO: 427 | SEQ ID NO: 434 | SEQ ID | SEQ ID NO: 404 |
| | NO: 431 | | | | NO: 255 | |
| SCT1.19 | GFTFSSY | GSVG | GYDWSGMD | SQGIRND | AAS | DYNYPY |
| | SEQ ID | SEQ ID NO: 435 | SEQ ID NO: 436 | SEQ ID NO: 437 | SEQ ID | SEQ ID NO: 404 |
| | NO: 431 | | | | NO: 255 | |
| SCT2.2 | GFTFSSY | FDGT | VAIQGRDYYID | SQGIRNG | AAS | DYNYPY |
| | SEQ ID | SEQ ID NO: 438 | SEQ ID NO: 439 | SEQ ID NO: 434 | SEQ ID | SEQ ID NO: 404 |
| | NO: 431 | | | | NO: 255 | |
| SCT2.3 | GFTFSSY | FDGT | VAIQGYYYYMD | SQDIRNG | TTS | DYNYPY |
| | SEQ ID | SEQ ID NO: 438 | SEQ ID NO: 440 | SEQ ID NO: 428 | SEQ ID | SEQ ID NO: 404 |
| | NO: 431 | | | | NO: 284 | |

Binds to CD4 D3

| | | | | | | |
|---|---|---|---|---|---|---|
| OKT4 | GYTFTNY | TNTG | GIYYDYGYYAMD | SESVDSYGNSF | LAS | NNEDPY |
| | SEQ ID | SEQ ID NO: 442 | SEQ ID NO: 443 | SEQ ID NO: 444 | SEQ ID | SEQ ID NO: 445 |
| | NO: 441 | | | | NO: 245 | |
| SCT1.20 | EIIFSTY | GSGD | GYNWNYMD | SQGIRND | EAS | DYTYPY |
| | SEQ ID | SEQ ID NO: 447 | SEQ ID NO: 448 | SEQ ID NO: 437 | SEQ ID | SEQ ID NO: 449 |
| | NO: 446 | | | | NO: 308 | |
| SCT1.21 | GLTESTE | GSGE | GYNWNYMD | SHGIRND | ETS | DYTYPY |
| | SEQ ID | SEQ ID NO: 451 | SEQ ID NO: 448 | SEQ ID NO: 452 | SEQ ID | SEQ ID NO: 449 |
| | NO: 450 | | | | NO: 313 | |
| SCT1.22 | GLTFSTF | GSGE | GYNWNYMD | SHGIRND | EAS | DYTYPY |
| | SEQ ID | SEQ ID NO: 451 | SEQ ID NO: 448 | SEQ ID NO: 452 | SEQ ID | SEQ ID NO: 449 |
| | NO: 450 | | | | NO: 308 | |
| SCT1.23 | GFTESTY | DNIG | NEDYYMD | SQGIRND | AAS | DYNYPR |
| C36Y | SEQ ID | SEQ ID NO: 454 | SEQ ID NO: 455 | SEQ ID NO: 437 | SEQ ID | SEQ ID NO: 456 |
| | NO: 453 | | | | NO: 255 | |
| SCT2.1 | GFTESTN | GSNG | RDNWNGFD | SHVIRND | AAS | DYNYPY |
| | SEQ ID | SEQ ID NO: 458 | SEQ ID NO: 459 | SEQ ID NO: 460 | SEQ ID | SEQ ID NO: 404 |
| | NO: 457 | | | | NO: 255 | |
| SCT2.8 | GHTLTEL | PRDG | GNEDYYFYYMD | SQDIDDD | EAT | HDHFPY |
| | SEQ ID | SEQ ID NO: 350 | SEQ ID NO: 461 | SEQ ID NO: 347 | SEQ ID | SEQ ID NO: 462 |
| | NO: 343 | | | | NO: 200 | |

TABLE A4
Illustrative CDRs for anti-CD4 binding domains (Honegger)

| Ab | | | | | | VL - |
|---|---|---|---|---|---|---|
| Name | VH - CDR1 | VH - CDR2 | VH - CDR3 | VL - CDR1 | VL - CDR2 | CDR3 |
| 1 | ASGFSFSNYA | INX₁X₂GSTFYX₃DSVKX₄ | HYGGSYDPMD | ASQDINKY | YTSX₆LHPGVPSR | YDNPLX₇ |
| | SEQ ID | X₁ is A, D or E | SEQ ID NO: 465 | SEQ ID NO: 466 | X₆ is I or T | X₇ is |
| | NO: 463 | X₂ is A, G, W or Y | | | SEQ ID | F, Q, Y |
| | | X₃ is G or S | | | NO: 467 | SEQ ID |
| | | X₄ is A or P | | | | NO: 327 |
| | | SEQ ID NO: 464 | | | | |
| 2 | ASGFSFSNYA | INX₁X₂GSTFYX₃DSVKX₄ | HYGGSYDPMD | ASQDINKY | YTSTLHPGVPSR | YDNPLY |
| | SEQ ID | X₁ is A, D or E | SEQ ID NO: 465 | SEQ ID NO: 466 | SEQ ID | SEQ ID |
| | NO: 463 | X₂ is A or G | | | NO: 469 | NO: 329 |
| | | X₃ is G or S | | | | |
| | | X₄ is A or P | | | | |
| | | SEQ ID NO: 468 | | | | |
| 3 | ASGFSFSNYA | INEX₂GSTFYADSVK | HYGGSYDPMD | ASQDINKY | YTSX₆LHPGVPSR | YDNPLX₇ |
| | SEQ ID | X₂ is W or Y | SEQ ID NO: 465 | SEQ ID NO: 466 | X₆ is I or T | X₇ is |
| | NO: 463 | SEQ ID NO: 470 | | | SEQ ID | F, Q, Y |
| | | | | | NO: 467 | SEQ ID |
| | | | | | | NO: 327 |
| 4 | ASGFSFSNYA | INEX₂GSTFYADSVK | HYGGSYDPMD | ASQDINKY | YTSTLHPGVPSR | YDNPLX₇ |
| | SEQ ID | X₂ is W or Y | SEQ ID NO: 465 | SEQ ID NO: 466 | SEQ ID | X₇ is |
| | NO: 463 | SEQ ID NO: 470 | | | NO: 469 | F, Q, Y |
| | | | | | | SEQ ID |
| | | | | | | NO: 327 |
| 5 | ASGFSFSNYA | INEX₂GSTFYADSVK | HYGGSYDPMD | ASQDINKY | YTSTLHPGVPSR | YDNPLY |
| | SEQ ID | X₂ is W or Y | SEQ ID NO: 465 | SEQ ID NO: 466 | SEQ ID | SEQ ID |
| | NO: 463 | SEQ ID NO: 470 | | | NO: 469 | NO: 329 |
| 6 | ASGFSFSNYA | INEGGSTFYADSVKX₄R | HYGGSYDPMD | ASQDINKY | YTSTLHPGVPSR | YDNPLY |
| | SEQ ID | X₄ is G or S | SEQ ID NO: 465 | SEQ ID NO: 466 | SEQ ID | SEQ ID |
| | NO: 463 | SEQ ID NO: 1132 | | | NO: 469 | NO: 329 |
| 7 | ASGFSFSNYA | INEGGSTFYADSVKSR | HYGGSYDPMD | ASQDINKY | YTSTLHPGVPSR | YDNPLY |
| | SEQ ID | SEQ ID NO: 471 | SEQ ID NO: 465 | SEQ ID NO: 466 | SEQ ID | SEQ ID |
| | NO: 463 | | | | NO: 469 | NO: 329 |
| 8 | ASGFSFSNYA | INEWGSTFYADSVKSR | HYGGSYDPMD | ASQDINKY | YTSTLHPGVPSR | YDNPLY |
| | SEQ ID | SEQ ID NO: 472 | SEQ ID NO: 465 | SEQ ID NO: 466 | SEQ ID | SEQ ID |
| | NO: 463 | | | | NO: 469 | NO: 329 |
| 9 | ASGFSFSNYA | INEYGSTFYADSVKSR | HYGGSYDPMD | ASQDINKY | YTSTLHPGVPSR | YDNPLY |
| | SEQ ID | SEQ ID NO: 473 | SEQ ID NO: 465 | SEQ ID NO: 466 | SEQ ID | SEQ ID |
| | NO: 463 | | | | NO: 469 | NO: 329 |
| 10 | ASGFSFSNYA | INEWGSTFYADSVKSR | HYGGSYDPMD | ASQDINKY | YTSILHPGVPSR | YDNPLQ |
| | SEQ ID | SEQ ID NO: 472 | SEQ ID NO: 465 | SEQ ID NO: 466 | SEQ ID | SEQ ID |
| | NO: 463 | | | | NO: 474 | NO: 334 |
| 11 | ASGFSFSNYA | INEYGSTFYADSVKSR | HYGGSYDPMD | ASQDINKY | YTSILHPGVPSR | YDNPLF |
| | SEQ ID | SEQ ID NO: 473 | SEQ ID NO: 465 | SEQ ID NO: 466 | SEQ ID | SEQ ID |
| | NO: 463 | | | | NO: 474 | NO: 335 |
| 12 | ASGFSFSNYA | INEGGSTFYADSVKGR | HYGGSYDPMD | ASQDINKY | YTSTLHPGVPSR | YDNPLY |
| | SEQ ID | SEQ ID NO: 475 | SEQ ID NO: 465 | SEQ ID NO: 466 | SEQ ID | SEQ ID |
| | NO: 463 | | | | NO: 469 | NO: 329 |
| 13 | ASGFSFSNYA | INDGGSTFYADSVKGR | HYGGSYDPMD | ASQDINKY | YTSTLHPGVPSR | YDNPLY |
| | SEQ ID | SEQ ID NO: 478 | SEQ ID NO: 465 | SEQ ID NO: 466 | SEQ ID | SEQ ID |
| | NO: 463 | | | | NO: 469 | NO: 329 |
| 14 | ASGFSFSNYA | INDGGSTFYADSVKGR | HYGGSYDPMD | ASQDINKY | YTSTLHPGVPSR | YDNPLY |
| | SEQ ID | SEQ ID NO: 478 | SEQ ID NO: 465 | SEQ ID NO: 466 | SEQ ID | SEQ ID |
| | NO: 463 | | | | NO: 469 | NO: 329 |
| 15 | ASGFSFSNYA | INAGGSTFYADSVKGR | HYGGSYDPMD | ASQDINKY | YTSTLHPGVPSR | YDNPLY |
| | SEQ ID | SEQ ID NO: 476 | SEQ ID NO: 465 | SEQ ID NO: 466 | SEQ ID | SEQ ID |
| | NO: 463 | | | | NO: 469 | NO: 329 |
| 16 | ASGFSFSNYA | INDAGSTFYADSVKGR | HYGGSYDPMD | ASQDINKY | YTSTLHPGVPSR | YDNPLY |
| | SEQ ID | SEQ ID NO: 477 | SEQ ID NO: 465 | SEQ ID NO: 466 | SEQ ID | SEQ ID |
| | NO: 463 | | | | NO: 469 | NO: 329 |
| 17 | ASGFSFSNYA | INDGGSTFYADSVKGR | HYGGSYDPMD | ASQDINKY | YTSTLHPGVPSR | YDNPLY |
| | SEQ ID | SEQ ID NO: 478 | SEQ ID NO: 465 | SEQ ID NO: 466 | SEQ ID | SEQ ID |
| | NO: 463 | | | | NO: 469 | NO: 329 |
| 18 | ASGFSFSNYA | INDGGSTFYADSVKGR | HYGGSYDPMD | ASQDINKY | YTSTLHPGVPSR | YDNPLY |
| | SEQ ID | SEQ ID NO: 478 | SEQ ID NO: 465 | SEQ ID NO: 466 | SEQ ID | SEQ ID |
| | NO: 463 | | | | NO: 469 | NO: 329 |
| 19 | ASGFSFSNYA | INDGGSTFYADSVKGR | HYGGSYDPMD | ASQDINKY | YTSTLHPGVPSR | YDNPLY |
| | SEQ ID | SEQ ID NO: 478 | SEQ ID NO: 465 | SEQ ID NO: 466 | SEQ ID | SEQ ID |
| | NO: 463 | | | | NO: 469 | NO: 329 |
| SCT1.17 | PSGFTFDDYG | INWNGDSTGYADSVRGR | DGAIGGMD | ASQDIRDD | SASTLQSGVPSR | DYNYPW |
| | SEQ ID | SEQ ID NO: 480 | SEQ ID NO: 481 | SEQ ID NO: 482 | SEQ ID | SEQ ID |
| | NO: 479 | | | | NO: 483 | NO: 343 |
| SCT1.25 | VSGHTLTELS | FDPRGGETIYAQKFQGR | GGDRDYYYYYMD | ASQDIDDD | EATTLVPGVPPR | HDSFPY |
| | SEQ ID | SEQ ID NO: 485 | SEQ ID NO: 486 | SEQ ID NO: 487 | SEQ ID | SEQ ID |
| | NO: 484 | | | | NO: 488 | NO: 348 |
| SCT1.26 | VSGFTLTKLS | FDPRDGERIYAQKFQGR | GGDYDYYYYYMG | ASQDIDDD | EATTLVPGIPPR | HDNFPY |
| | SEQ ID | SEQ ID NO: 490 | SEQ ID NO: 491 | SEQ ID NO: 487 | SEQ ID | SEQ ID |
| | NO: 489 | | | | NO: 492 | NO: 352 |
| SCT1.27 | VSGHTVTELS | FHPRDDEIIYAQKFQGR | GGNPDYYFYYMD | ASQDIDDD | EATTLVPGIPPR | HDNFPL |
| | SEQ ID | SEQ ID NO: 494 | SEQ ID NO: 495 | SEQ ID NO: 487 | SEQ ID | SEQ ID |
| | NO: 493 | | | | NO: 492 | NO: 356 |
| SCT1.28 | VSGHTLTELS | FHPRDGETIYAQKFQGR | GGNKDYYFYYMD | ASQDIDDD | EATTLVPGIPPR | HDNFPL |
| | SEQ ID | SEQ ID NO: 496 | SEQ ID NO: 497 | SEQ ID NO: 487 | SEQ ID | SEQ ID |
| | NO: 484 | | | | NO: 492 | NO: 356 |
| SCT1.29 | VSGYTLTALS | FNPRDGETIYAQKFQGR | GGDSDYYYYYMD | ASQDIDDD | EATTLVPGIPPR | HDNFPY |
| | SEQ ID | SEQ ID NO: 499 | SEQ ID NO: 500 | SEQ ID NO: 487 | SEQ ID | SEQ ID |
| | NO: 498 | | | | NO: 492 | NO: 352 |
| SCT1.30 | VSGHTLTELS | FNPRDDETIYTQKFQGR | GGNKDYYFYYMD | ASQDIDDD | EATTLVPGIPPR | HDNFPL |
| | SEQ ID | SEQ ID NO: 501 | SEQ ID NO: 497 | SEQ ID NO: 487 | SEQ ID | SEQ ID |
| | NO: 484 | | | | NO: 492 | NO: 356 |
| SCT1.31 | VSGHTLTELS | FDPKYGETTYAQKFQDR | GGNYEYFYYYMD | ASQDIDDD | EATTLVPGIPPR | HDNFPY |
| | SEQ ID | SEQ ID NO: 502 | SEQ ID NO: 503 | SEQ ID NO: 487 | SEQ ID | SEQ ID |
| | NO: 484 | | | | NO: 492 | NO: 352 |
| SCT1.32 | VSGHTLTELS | FDPKYGETMYAQKFQGR | GGSPDYFYYYMD | ASQDIDDD | EATTLVPGIPRR | HDNFPY |
| | SEQ ID | SEQ ID NO: 504 | SEQ ID NO: 505 | SEQ ID NO: 487 | SEQ ID | SEQ ID |
| | NO: 484 | | | | NO: 506 | NO: 352 |
| SCT1.33 | VSGHTLTELS | FDPRNGETIYAQKFQGR | GGSPDYYYWYMD | ASQDIDDD | EATTLVPGIPPR | HDNFPY |
| | SEQ ID | SEQ ID NO: 507 | SEQ ID NO: 508 | SEQ ID NO: 487 | SEQ ID | SEQ ID |
| | NO: 484 | | | | NO: 492 | NO: 352 |
| SCT1.34 | VSGHTLTELS | FDPRNGETIYAQKFQGR | GGSPDYYFWYMD | ASQDIDDD | EATTLVPGIPPR | HDNFPY |
| | SEQ ID | SEQ ID NO: 507 | SEQ ID NO: 509 | SEQ ID NO: 487 | SEQ ID | SEQ ID |
| | NO: 484 | | | | NO: 492 | NO: 352 |
| SCT1.45 | ASGYTFTSYD | MNPNSGNTGSTQKFQGR | GVLKGDYYYMD | ASQSISSSY | GSSTRATGIPAR | DDHLPL |
| | SEQ ID | SEQ ID NO: 511 | SEQ ID NO: 512 | SEQ ID NO: 513 | SEQ ID | SEQ ID |
| | NO: 510 | | | | NO: 514 | NO: 369 |
| SCT1.46 | ASGYTFTSYD | VNPNSGNTGSTQKFQGR | GVLKGDYYYMD | ASQSISSTY | GSSTRATGIPAR | DDHLPL |
| | SEQ ID | SEQ ID NO: 515 | SEQ ID NO: 512 | SEQ ID NO: 516 | SEQ ID | SEQ ID |
| | NO: 510 | | | | NO: 514 | NO: 369 |
| SCT1.47 | ASGYTFTSYD | VNPNSGNTGSTQKFQGR | GVLKGDYYYMD | ASQSISSTY | GSSTRATGIPAR | DDHLPL |
| | SEQ ID | SEQ ID NO: 515 | SEQ ID NO: 512 | SEQ ID NO: 516 | SEQ ID | SEQ ID |
| | NO: 510 | | | | NO: 514 | NO: 369 |
| SCT1.48 | ASGYTFTSYD | VNPNSGNTGSTQKFQGR | GVLKGDYYYMD | ASQSISSTY | GSSTRATGIPAR | DDHLPL |
| | SEQ ID | SEQ ID NO: 515 | SEQ ID NO: 512 | SEQ ID NO: 516 | SEQ ID | SEQ ID |
| | NO: 510 | | | | NO: 514 | NO: 369 |
| Keliximab | VSGGSISGDYY | IYGSGGGTNYNPSLNNR | NILKYLHWLL | GDNVGRKS | ADSERPSGIPAR | WDSTADH |
| | SEQ ID | SEQ ID NO: 518 | SEQ ID NO: 519 | SEQ ID NO: 374 | SEQ ID | W |
| | NO: 517 | | | | NO: 520 | SEQ ID |
| | | | | | | NO: 375 |
| UB-421 | ASGYTFTDYV | IYPGSGSAYSNAKFKDR | RGNGTGFA | AGQSVDYDGDSY | VASNLESGIPAR | SYKDPL |
| | SEQ ID | SEQ ID NO: 522 | SEQ ID NO: 523 | SEQ ID NO: 524 | SEQ ID | SEQ ID |
| | NO: 521 | | | | NO: 525 | NO: 380 |

Binds to CD4 D2

| | | | | | | |
|---|---|---|---|---|---|---|
| ibalizumab | ASGYTFTSYV | INPYNDGTDYDEKFKGK | EKDNYATGAWFA | SSQSLLYSTNQKNY | WASTRESGVPDR | YYSYR |
| | SEQ ID | SEQ ID NO: 527 | SEQ ID NO: 528 | SEQ ID NO: 529 | SEQ ID | SEQ ID |
| | NO: 526 | | | | NO: 530 | NO: 384 |
| tregalizumab | ASGFSFSDCR | ISVKSENYGANYAESVRGR | SYYRYDVGAWFA | ASKSVSTSGYSY | LASILESGVPDR | SRELPW |
| | SEQ ID | SEQ ID NO: 532 | SEQ ID NO: 533 | SEQ ID NO: 534 | SEQ ID | SEQ ID |
| | NO: 531 | | | | NO: 535 | NO: 389 |
| SCT1.1 | VSGYTLTELS | FDPEDGKTIYAPKFQGR | GHNWNDGYYFYYYMD | SSQSLLHTDGKTY | EVSNRFSGVPDR | SLQLPL |
| | SEQ ID | SEQ ID NO: 537 | SEQ ID NO: 538 | SEQ ID NO: 539 | SEQ ID | SEQ ID |
| | NO: 536 | | | | NO: 540 | NO: 394 |
| SCT1.4 | ASGFTFSDYY | ISSSGNTIFYVDSVEGR | EGYNNYNYSYYYFMD | ASQSVSRK | AASTRATGIPAR | YNNWPY |
| | SEQ ID | SEQ ID NO: 542 | SEQ ID NO: 543 | SEQ ID NO: 544 | SEQ ID | SEQ ID |
| | NO: 541 | | | | NO: 545 | NO: 399 |
| SCT1.5 | ASGFTVSSFG | IWYDGTNKYYADSVKGR | EIAVDGTDYYMD | ASQGIRSG | AASTLQSGVPSR | DYNYPY |
| | SEQ ID | SEQ ID NO: 547 | SEQ ID NO: 548 | SEQ ID NO: 549 | SEQ ID | SEQ ID |
| | NO: 546 | | | | NO: 550 | NO: 404 |
| SCT2.9 | ASGFTFSDYY | ISSSGLTIFYVDSVKGR | EGYSGFDDHYYYYTD | ASQSVSSN | GASIRATGIPAR | YKNWPY |
| | SEQ ID | SEQ ID NO: 551 | SEQ ID NO: 552 | SEQ ID NO: 553 | SEQ ID | SEQ ID |
| | NO: 541 | | | | NO: 554 | NO: 408 |
| SCT2.10 | ASGFTFSDYY | ISSSGSTIFYTDSVKGR | EDYSNYEDYYYYYTG | ASQSVSSK | NASTRATGIPAR | YNNWPY |
| | SEQ ID | SEQ ID NO: 555 | SEQ ID NO: 556 | SEQ ID NO: 557 | SEQ ID | SEQ ID |
| | NO: 541 | | | | NO: 558 | NO: 399 |
| SCT2.11 | ASGFTFSDYY | ISSSGITIFYTDSVKGR | EDYSNYEDHYYYYTG | ASQSVSNK | NASTRATSIPAR | YNNWPY |
| | SEQ ID | SEQ ID NO: 559 | SEQ ID NO: 560 | SEQ ID NO: 561 | SEQ ID | SEQ ID |
| | NO: 541 | | | | NO: 562 | NO: 399 |
| SCT2.11 | ASGFTFSDYY | ISSSGITIFYTDSVKGR | EDYSNYEDHYYYYTG | ASQSVSNK | GASTRATSIPAR | YNNWPY |
| VL | SEQ ID | SEQ ID NO: 559 | SEQ ID NO: 560 | SEQ ID NO: 561 | SEQ ID | SEQ ID |
| N50G | NO: 541 | | | | NO: 563 | NO: 399 |
| SCT2.12 | ASGFTFSDYY | ISSSGKTIFYTDSVKGR | EDYSNYEDYYYYYTG | ASQSVSSK | NASTRATGIPAR | YKNWPY |
| | SEQ ID | SEQ ID NO: 564 | SEQ ID NO: 556 | SEQ ID NO: 557 | SEQ ID | SEQ ID |
| | NO: 541 | | | | NO: 558 | NO: 408 |
| SCT2.12 | ASGFTESDYY | ISSSGKTIFYTDSVKGR | EDYSNYEDYYYYYTG | ASQSVSSK | GASTRATGIPAR | YKNWPY |
| VL | SEQ ID | SEQ ID NO: 564 | SEQ ID NO: 556 | SEQ ID NO: 557 | SEQ ID | SEQ ID |
| N50G | NO: 541 | | | | NO: 565 | NO: 408 |
| SCT2.13 | ASGFTFSDYY | ISISGQTIYYGDSVKGR | EGYSNYGVKYYYYMD | ASQSISSN | NASTRATDIPAR | YNAWTY |
| | SEQ ID | SEQ ID NO: 566 | SEQ ID NO: 567 | SEQ ID NO: 568 | SEQ ID | SEQ ID |
| | NO: 541 | | | | NO: 569 | NO: 419 |
| SCT2.14 | ASGFTFSDHY | ISISGNTIYYTDSVKGR | EGYSSSSRGDYSYYTD | ASQSVSSN | GASTRATGIPAR | YKNWPY |
| | SEQ ID | SEQ ID NO: 571 | SEQ ID NO: 572 | SEQ ID NO: 553 | SEQ ID | SEQ ID |
| | NO: 570 | | | | NO: 565 | NO: 408 |
| SCT2.15 | ASGFTFSDYY | ISISGRTIYYIDSVKGR | EGYSSSSRGDYSYYTD | ASQGVSSN | GASTRATDIPAR | YKNWPY |
| | SEQ ID | SEQ ID NO: 573 | SEQ ID NO: 572 | SEQ ID NO: 574 | SEQ ID | SEQ ID |
| | NO: 541 | | | | NO: 575 | NO: 408 |

Binds to CD4 D2 and/or D3

| | | | | | | |
|---|---|---|---|---|---|---|
| SCT1.6 | ASGFTVSSYG | IWFDGSNKYYADSLTGL | EVALEGYYYYMD | ASQDIRNG | TTSSLQTGVPSR | DYNYPY |
| | SEQ ID | SEQ ID NO: 577 | SEQ ID NO: 578 | SEQ ID NO: 579 | SEQ ID | SEQ ID |
| | NO: 576 | | | | NO: 580 | NO: 404 |
| SCT1.7 | ASGFTVSSYG | LWFDGSNKFYADSVKGR | ELALEGYYYYMD | ASQDIRNG | TTSSLQSGVPSR | DYNYPY |
| | SEQ ID | SEQ ID NO: 581 | SEQ ID NO: 582 | SEQ ID NO: 579 | SEQ ID | SEQ ID |
| | NO: 576 | | | | NO: 583 | NO: 404 |
| SCT1.8 | ASGFTVSSYG | LWFDGSNQFYADSVKGR | EIALEGYYYYMD | SSQDIRNG | TTSSLQSGVPSR | DYNYPY |
| | SEQ ID | SEQ ID NO: 584 | SEQ ID NO: 585 | SEQ ID NO: 586 | SEQ ID | SEQ ID |
| | NO: 576 | | | | NO: 583 | NO: 404 |
| SCT1.9 | ASGFTFSSYG | IWYDGSNKYYADSVKGR | EVALEGYYYYMD | ASQDIRNG | TTSNLQSGVPSR | DYNYPY |
| | SEQ ID | SEQ ID NO: 588 | SEQ ID NO: 578 | SEQ ID NO: 579 | SEQ ID | SEQ ID |
| | NO: 587 | | | | NO: 589 | NO: 404 |
| SCT1.10 | ASGFTFSSYG | IWYDGNNKYYADSVKGR | EVALEGYYYYMD | ASQGIRNG | AASSLQSGVPSS | DYNYPY |
| | SEQ ID | SEQ ID NO: 590 | SEQ ID NO: 578 | SEQ ID NO: 591 | SEQ ID | SEQ ID |
| | NO: 587 | | | | NO: 592 | NO: 404 |
| SCT1.19 | ASGFTFSSYA | ISGSVGSTYYADSVKGR | EGYDWSGMD | ASQGIRND | AASSLQSGVPSR | DYNYPY |
| | SEQ ID | SEQ ID NO: 594 | SEQ ID NO: 595 | SEQ ID NO: 596 | SEQ ID | SEQ ID |
| | NO: 593 | | | | NO: 597 | NO: 404 |
| SCT2.2 | ASGFTFSSYG | IWFDGTTRFYADSVKGR | EVAIQGRDYYID | ASQGIRNG | AASSLQSGVPSR | DYNYPY |
| | SEQ ID | SEQ ID NO: 598 | SEQ ID NO: 599 | SEQ ID NO: 591 | SEQ ID | SEQ ID |
| | NO: 587 | | | | NO: 597 | NO: 404 |
| SCT2.3 | ASGFTFSSYG | IWFDGTNKFYADSVKGR | EVAIQGYYYYMD | ASQDIRNG | TTSNLQNGVPSR | DYNYPY |
| | SEQ ID | SEQ ID NO: 600 | SEQ ID NO: 601 | SEQ ID NO: 579 | SEQ ID | SEQ ID |
| | NO: 587 | | | | NO: 602 | NO: 404 |

Binds to CD4 D3

| | | | | | | |
|---|---|---|---|---|---|---|
| OKT4 | ASGYTFTNYG | INTNTGEPTYAEEFKGR | LGIYYDYGYYAMD | ASESVDSYGNSF | LASNLESGVPAR | NNEDPY |
| | SEQ ID | SEQ ID NO: 604 | SEQ ID NO: 605 | SEQ ID NO: 606 | SEQ ID | SEQ ID |
| | NO: 603 | | | | NO: 607 | NO: 445 |
| SCT1.20 | ASEIIFSTYA | ISGSGDNTYYADSVKGR | EGYNWNYMD | ASQGIRND | EASSLQSGVPSR | DYTYPY |
| | SEQ ID | SEQ ID NO: 609 | SEQ ID NO: 610 | SEQ ID NO: 596 | SEQ ID | SEQ ID |
| | NO: 608 | | | | NO: 611 | NO: 449 |
| SCT1.21 | ASGLTESTFA | ISGSGENTYYADSVKGR | EGYNWNYMD | ASHGIRND | ETSSLQSGVPSR | DYTYPY |
| | SEQ ID | SEQ ID NO: 613 | SEQ ID NO: 610 | SEQ ID NO: 614 | SEQ ID | SEQ ID |
| | NO: 612 | | | | NO: 615 | NO: 449 |
| SCT1.22 | ASGLTFSTFA | ISGSGENTYYADSVKGR | EGYNWNYMD | ASHGIRND | EASSLQSGVPSR | DYTYPY |
| | SEQ ID | SEQ ID NO: 613 | SEQ ID NO: 610 | SEQ ID NO: 614 | SEQ ID | SEQ ID |
| | NO: 612 | | | | NO: 611 | NO: 449 |
| SCT1.23 | ASGFTFSTYA | ISDNIGNTYYADSVKGR | DNEDYYMD | ASQGIRND | AASTLQSGVPSR | DYNYPR |
| | SEQ ID | SEQ ID NO: 617 | SEQ ID NO: 618 | SEQ ID NO: 596 | SEQ ID | SEQ ID |
| | NO: 616 | | | | NO: 550 | NO: 456 |
| SCT2.1 | ASGFTESTNA | ISGSNGNTYYADSVKGR | ERDNWNGFD | ASHVIRND | AASSLQSGVPSR | DYNYPY |
| | SEQ ID | SEQ ID NO: 620 | SEQ ID NO: 621 | SEQ ID NO: 622 | SEQ ID | SEQ ID |
| | NO: 619 | | | | NO: 597 | NO: 404 |
| SCT2.8 | VSGHTLTELS | FDPRDGQIIYAEKFQGR | GGNEDYYFYYMD | ASQDIDDD | EATTLVPGIPPR | HDHFPY |
| | SEQ ID | SEQ ID NO: 623 | SEQ ID NO: 624 | SEQ ID NO: 487 | SEQ ID | SEQ ID |
| | NO: 484 | | | | NO: 492 | NO: 462 |

TABLE B
Illustrative VH/VL for anti-CD4 binding domains

| | | |
|---|---|---|
| Ab | | |
| Name | VH | VL |

Binds to CD4 D1

| | | |
|---|---|---|
| 1 | SEQ ID NO: 625 | SEQ ID NO: 626 |
| | EVQLLESGGGLVQPGGSLRLSCAASGFSFSNYAMSWVRQAPG | DIQMTQSPSSLSASVGDRVTITCX₅ASQDINKYIAWYQ |
| | KGLEWVASINX₁X₂GSTFYX₃DSVKX₄RFTISRDNSKNTLYLE | QKPGKGPKLLIHYTSX₆LHPGVPSRFSGSGSGTDYTLT |
| | MNSLRAEDTAVFYCSRHYGGSYDPMDYWGQGTTVTVSS | ISSLQPEDFATYYCLQYDNPLX₇TFGQGTKLEIK |
| | X₁ is A, D or E | X₅ is K or R |
| | X₂ is A, G, W or Y | X₆ is I or T |
| | X₃ is A or P | X₇ is F, Q or Y |
| | X₄ is G or S | SEQ ID NO: 628 |
| 2 | SEQ ID NO: 627 | |
| | EVQLLESGGGLVQPGGSLRLSCAASGFSFSNYAMSWVRQAPG | DIQMTQSPSSLSASVGDRVTITCX₅ASQDINKYIAWYQ |
| | KGLEWVASINX₁X₂GSTFYX₃DSVKX₄RFTISRDNSKNTLYLE | QKPGKGPKLLIHYTSTLHPGVPSRFSGSGSGTDYTLTI |
| | MNSLRAEDTAVFYCSRHYGGSYDPMDYWGQGTTVTVSS | SSLQPEDFATYYCLQYDNPLYTFGQGTKLEIK |
| | X₁ is A, D or E | X₅ is K or R |
| | X₂ is A or G | |
| | X₃ is A or P | |
| | X₄ is G or S | |
| 3 | SEQ ID NO: 629 | SEQ ID NO: 630 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEX₂GSTFYADSVKSRFTISRDNSKNTLYLEMN | KPGKGPKLLIHYTSX₆LHPGVPSRFSGSGSGTDYTLTI |
| | SLRAEDTAVFYCSRHYGGSYDPMDYWGQGTTVTVSS | SSLQPEDFATYYCLQYDNPLX-TFGQGTKLEIK |
| | X₂ is W or Y | X₆ is I or T |
| | | X₇ is F, Q or Y |
| 4 | SEQ ID NO: 629 | SEQ ID NO: 631 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEX₂GSTFYADSVKSRFTISRDNSKNTLYLEMN | KPGKGPKLLIHYTSILHPGVPSRFSGSGSGTDYTLTIS |
| | SLRAEDTAVFYCSRHYGGSYDPMDYWGQGTTVTVSS | SLQPEDFATYYCLQYDNPLX₆TFGQGTKLEIK |
| | X₂ is W or Y | X₇ is F, Q or Y |
| 5 | SEQ ID NO: 629 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEX₂GSTFYADSVKSRFTISRDNSKNTLYLEMN | KPGKGPKLLIHYTSTLHPGVPSRFSGSGSGTDYTLTIS |
| | SLRAEDTAVFYCSRHYGGSYDPMDYWGQGTTVTVSS | SLQPEDFATYYCLQYDNPLYTFGQGTKLEIK |
| | X₂ is W or Y | |
| 6 | SEQ ID NO: 633 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEGGSTFYADSVKX₄RFTISRDNSKNTLYLEMN | KPGKGPKLLIHYTSTLHPGVPSRFSGSGSGTDYTLTIS |
| | SLRAEDTAVFYCSRHYGGSYDPMDYWGQGTTVTVSS | SLQPEDFATYYCLQYDNPLYTFGQGTKLEIK |
| | X₄ is G or S | |
| 7 | SEQ ID NO: 634 | SEQ ID NO: 632 |
| | EVQLLESGGGLVQPGGSLRLSCAASGESFSNYAMSWVRQAPG | DIQMTQSPSSLSASVGDRVTITCRASQDINKYIAWYQQ |
| | KGLEWVASINEGGSTFYADSVKSRFTISRDNSKNTLYLEMNS | KPGKGPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 8 | SEQ ID NO: 635 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEWGSTFYADSVKSRFTISRDNSKNTLYLEMNS | KPGKGPKLLIHYTSTLHPGVPSRESGSGSGTDYTLTIS |
| | LRAEDTAVFYCSRHYGGSYDPMDYWGQGTTVTVSS | SLQPEDFATYYCLQYDNPLYTFGQGTKLEIK |
| 9 | SEQ ID NO: 636 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEYGSTFYADSVKSRFTISRDNSKNTLYLEMNS | KPGKGPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 10 | SEQ ID NO: 635 | SEQ ID NO: 637 |
| | EVQLLESGGGLVQPGGSLRLSCAASGESFSNYAMSWVRQAPG | DIQMTQSPSSLSASVGDRVTITCRASQDINKYIAWYQQ |
| | KGLEWVASINEWGST FYADSVKSRFTISRDNSKNTLYLEMNS | KPGKGPKLLIHYTSILHPGVPSRFSGSGSGTDYTLTIS |
| | LRAEDTAVFYCSRHYGGSYDPMDYWGQGTTVTVSS | SLQPEDFATYYCLQYDNPLQTFGQGTKLEIK |
| 11 | SEQ ID NO: 636 | SEQ ID NO: 638 |
| | EVQLLESGGGLVQPGGSLRLSCAASGESFSNYAMSWVRQAPG | DIQMTQSPSSLSASVGDRVTITCRASQDINKYIAWYQQ |
| | KGLEWVASINEYGSTFYADSVKSRFTISRDNSKNTLYLEMNS | KPGKGPKLLIHYTSILHPGVPSRFSGSGSGTDYTLTIS |
| | LRAEDTAVFYCSRHYGGSYDPMDYWGQGTTVTVSS | SLQPEDFATYYCLQYDNPLFTFGQGTKLEIK |
| 12 | SEQ ID NO: 639 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINEGGSTFYADSVKGRFTISRDNSKNTLYLQMNS | KPGKGPKLLIHYTSTLHPGVPSRESGSGSGTDYTLTIS |
| | LRAEDTAVFYCSRHYGGSYDPMDYWGQGTTVTVSS | SLQPEDFATYYCLQYDNPLYTFGQGTKLEIK |
| 13 | SEQ ID NO: 640 | SEQ ID NO: 632 |
| | EVQLLESGGGLVQPGGSLRLSCAASGESFSNYAMSWVRQAPG | DIQMTQSPSSLSASVGDRVTITCRASQDINKYIAWYQQ |
| | KGLEWVASINDGGSTFYADSVKGRFTISRDNSKNTLYLQMNS | KPGKGPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 14 | SEQ ID NO: 641 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINDGGSTFYADSVKGRFTISRDNSKNTLYLQMNS | KPGKGPKLLIHYTSTLHPGVPSRFSGSGSGTDYTLTIS |
| | LRAEDTAVYYCSRHYGGSYDPMDYWGQGTTVTVSS | SLQPEDFATYYCLQYDNPLYTFGQGTKLEIK |
| 15 | SEQ ID NO: 642 | SEQ ID NO: 632 |
| | EVQLLESGGGLVQPGGSLRLSCAASGESFSNYAMSWVRQAPG | DIQMTQSPSSLSASVGDRVTITCRASQDINKYIAWYQQ |
| | KGLEWVASINAGGSTFYADSVKGRFTISRDNSKNTLYLQMNS | KPGKGPKLLIHYTSTLHPGVPSRESGSGSGTDYTLTIS |
| | LRAEDTAVFYCSRHYGGSYDPMDYWGQGTTVTVSS | SLQPEDFATYYCLQYDNPLYTFGQGTKLEIK |
| 16 | SEQ ID NO: 643 | SEQ ID NO: 632 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINDAGST FYADSVKGRFTISRDNSKNTLYLQMNS | KPGKGPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 17 | SEQ ID NO: 640 | SEQ ID NO: 644 |
| | EVQLLESGGGLVQPGGSLRLSCAASGFSFSNYAMSWVRQAPG | DIQMTQSPSSLSASVGDRVTITCRASQDINKYIAWYQQ |
| | KGLEWVASINDGGSTFYADSVKGRFTISRDNSKNTLYLQMNS | KPGKAPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 18 | SEQ ID NO: 640 | SEQ ID NO: 645 |
| | EVQLLESGGGLVQPGGSLRLSCAASGFSFSNYAMSWVRQAPG | DIQMTQSPSSLSASVGDRVTITCRASQDINKYIAWYQH |
| | KGLEWVASINDGGSTFYADSVKGRFTISRDNSKNTLYLQMNS | KPGKAPKLLIHYTSTLHPGVPSRFSGSGSGTDYTLTIS |
| | LRAEDTAVFYCSRHYGGSYDPMDYWGQGTTVTVSS | SLQPEDFATYYCLQYDNPLYTFGQGTKLEIK |
| 19 | SEQ ID NO: 640 | SEQ ID NO: 646 |
| | EVQLLESGGGLVQPGGSLRLSCAASGFSFSNYAMSWVROAPG | DIQMTQSPSSLSASVGDRVTITCRASQDINKYIAWYQH |
| | KGLEWVASINDGGSTFYADSVKGRFTISRDNSKNTLYLQMNS | KPGKGPKLLIHYTSTLHPGVPSRESGSGSGTDYTLTIS |
| | LRAEDTAVFYCSRHYGGSYDPMDYWGQGTTVTVSS | SLQPEDFATYYCLQYDNPLYTFGQGTKLEIK |
| SCT1.17 | SEQ ID NO: 647 | SEQ ID NO: 648 |
| | EVQLVESGGGVVRPGGSLRLSCAPSGFTFDDYGMSWVRQAPG | AIQMTQSPSSLSASVGDRVTITCRASQDIRDDLGWYQQ |
| | KGLEWVSGINWNGDSTGYADSVRGRFTISRDNAKNSLYLQMN | KPGKAPKLLIYSASTLQSGVPSRFSGSESGTDFTLTIS |
| | SLRAEDTALYHCARDGAIGGMDVWGKGTTVTVSS | SLQPEDFAAYYCLQDYNYPWTFGQGTRVEIK |
| SCT1.25 | SEQ ID NO: 649 | SEQ ID NO: 650 |
| | QVQLVQSGAEVRKPGASVKVSCKVSGHTLTELSMHWIRQAPG | ETTLTQSPAFMSATPGDKVNISCKASQDIDDDMNWFOR |
| | EGLEWMGYFDPRGGETIYAQKFQGRVTLTEDTSTDTAYMELT | KPGEAAIFIIQEATTLVPGVPPRFSGSGYGTDFTLTIS |
| | SLTSEDTAVYYCATGGDRDYYYYYMDVWGKGTTVTVSS | NIESEDAAYYFCLQHDSFPYTFGQGTKLEIK |
| SCT1.26 | SEQ ID NO: 651 | SEQ ID NO: 652 |
| | QVQLLQSGAEVKRPGASVRVSCKVSGFTLTKLSMHWVRQAPG | ETTLTQSPAFMSATPGDKVNISCKASQDIDDDLNWYQQ |
| | KGLEWMGFFDPRDGERIYAQKFQGRVTMTEDTSTDTAYMELS | KPGEAAIFIVQEATTLVPGIPPRFSGSGYGTDFTLTIN |
| | SLRSEDTAVYYCATGGDYDYYYYYMGVWGKGTTVTVSS | NIESEDAAYYFCLQHDNFPYTFGQGTKLEIK |
| SCT1.27 | SEQ ID NO: 653 | SEQ ID NO: 654 |
| | QVQLVQSGAEVKKPGASVKVSCKVSGHTVTELSMHWVRQAPG | ETTLTQSPAFMSATPGDKVNISCKASQDIDDDMNWYQQ |
| | KGLEWMGSFHPRDDEIIYAQKFQGRVTMTEDSFTDTAYMELS | KPGEAAIFIIQEATTLVPGIPPRESGSGYGTDFTLTIN |
| | SLTSEDTAVYYCATGGNPDYYFYYMDVWGKGTPVTVSS | NIESEDAAYYFCLQHDNFPLTFGQGTKVEIK |
| SCT1.28 | SEQ ID NO: 655 | SEQ ID NO: 656 |
| | QVQLLQSGAEVKKPGASVKVSCKVSGHTLTELSMHWVRQAPG | ETTLTQSPAFMSATPGDKVNISCKASQDIDDDMNWYQQ |
| | KGLEWMGS FHPRDGETIYAQKFQGRVTLTEDTSKDTAFMELS | KPGEAAIFIIQEATTLVPGIPPRESGSGYGTDFTLTIN |
| | RLRSEDTAVYYCATGGNKDYYFYYMDVWGKGTTVTVSS | NIESEDAAYYFCLQHDNFPLTFGGGTKVEIK |
| SCT1.29 | SEQ ID NO: 657 | SEQ ID NO: 658 |
| | QVQLVQSGAEVKKPGASVKVSCKVSGYTLTALSMHWVRQAPG | ETTLTQSPAFMSATPGDKVNISCKASQDIDDDMNWYQQ |
| | KGLEWMGYFNPRDGETIYAQKFQGRVTMTEDTFTDTAYMELS | KPGEAAIFIIQEATTLVPGIPPRESGSGYGTDETLIIN |
| | SLRSEDTAIYYCATGGDSDYYYYYMDVWGKGTTVTVSS | NIESEDAAYYFCLQHDNFPYTFGQGTKLEIK |
| SCT1.30 | SEQ ID NO: 659 | SEQ ID NO: 660 |
| | QVQLVQSGAEVKKPGASVKVSCKVSGHTLTELSMHWVRQAPG | ETTLTQSPAFMSATPGDKVTISCKASQDIDDDMNWYQQ |
| | TGLEWMGYFNPRDDETIYTQKFQGRVTMTEDTSTDTAYMELR | KPGEAAIFIIQEATTLVPGIPPRESGSGYGTDFTLTIN |
| | SLRSEDTAVYYCATGGNKDYYFYYMDVWGKGTTVTVSS | NIESEDAAYYFCLQHDNFPLTFGQGTKVEIK |
| SCT1.31 | SEQ ID NO: 661 | SEQ ID NO: 662 |
| | QVQLVQSGAEVKKPGASVKVSCKVSGHTLTELSMHWVRQAPG | ETTLTQSPAFMSATPGDKVNISCKASQDIDDDMNWYQQ |
| | KGLEWMGVFDPKYGETTYAQKFODRVTMTEDTSTDTAYMELS | KPGEAAIFIIQEATTLVPGIPPRESGSGYGTDFTLTIN |
| | SLRSEDTAVYYCATGGNYEYFYYYMDVWGKGTTVTVSS | NIESEDAAYYFCLQHDNFPYTFGQGTKLEIK |
| SCT1.32 | SEQ ID NO: 663 | SEQ ID NO: 664 |
| | QVQLIQSGAEVKKPGASVKVSCKVSGHTLTELSMHWVRQAPG | ETTLTQSPAFMSATPGDKVNISCKASQDIDDDMNWYQQ |
| | KGLEWMGIFDPKYGETMYAQKFQGRVTLTEDTSTDTAYMELS | KPGEAAIFVIQEATTLVPGIPRRESGSGYGTDFTLTIN |
| | SLRSEDTAVYYCATGGSPDYFYYYMDVWGKGTTVTVSS | NIESEDAAYYFCLQHDNFPYTFGQGTKLEIK |
| SCT1.33 | SEQ ID NO: 665 | SEQ ID NO: 666 |
| | QVQLLQSGAEVKKPGASVKVSCKVSGHTLTELSMHWVRQAPG | ETTLTQSPAFMSATPGDKVNISCKASQDIDDDMNWYQQ |
| | KGLEWMGIFDPRNGETIYAQKFQGRVTMTEDTSTDTAYMELS | KPGEPAIFIIQEATTLVPGIPPRESGSGYGTDFTLTIN |
| | SLRSEDTAVYYCATGGSPDYYYWYMDVWGKGTTVTVSS | NIESEDAAYYFCLQHDNFPYTFGQGTKLEIK |
| SCT1.34 | SEQ ID NO: 667 | SEQ ID NO: 666 |
| | QVQLLQSGAEVKKPGASVKVSCKVSGHTLTELSMHWVRQAPG | ETTLTQSPAFMSATPGDKVNISCKASQDIDDDMNWYQQ |
| | KGLEWMGIFDPRNGETIYAQKFQGRVTMTEDTSTDTAYMELS | KPGEPAIFIIQEATTLVPGIPPRESGSGYGTDFTLTIN |
| | SLSSEDTAVYYCATGGSPDYYFWYMDVWGKGTTVTVSS | NIESEDAAYYFCLQHDNFPYTFGQGTKLEIK |
| SCT1.45 | SEQ ID NO: 668 | SEQ ID NO: 669 |
| | QVQLVQSGAEVKKPGASVKVSCKASGYTFTSYDINWVRQATG | EIVMTQSPATLSLSPGERATLSCRASQSISSSYLSWYQ |
| | QGLEWMGWMNPNSGNTGSTQKFQGRVTMTRNTSITTAYMELS | QKPGQAPRLLIYGSSTRATGIPARFSGSGSGTDFTLTI |
| | SLRSEDTAVYYCARGVLKGDYYYMDVWGKGTTVTVSS | SSLQPEDFAVYYCQQDDHLPLTFGGGTKVEIK |
| SCT1.46 | SEQ ID NO: 670 | SEQ ID NO: 671 |
| | QVQLVHSGAEVKKPGASVKVSCKASGYTFTSYDINWVRQATG | EIVMTQSPATLSLSPGERATLSCRASQSISSTYLSWYQ |
| | QGLEWMGWVNPNSGNTGSTQKFQGRVTMTRNTSISTAYMELS | QKPGQAPRLLIYGSSTRATGIPARFSGSGSGTDFTLTI |
| | SLRSEDTAVYYCARGVLKGDYYYMDVWGKGTTVTVSS | SSLQPEDFAVYYCQQDDHLPLTFGGGTKVEIK |
| SCT1.47 | SEQ ID NO: 672 | SEQ ID NO: 673 |
| | QVQLVQSGAEVKKPGASVKVSCKASGYTFTSYDINWVRQATG | EIVMTQSPATLSLSPGERATLSCRASQSISSTYLSWYQ |
| | QGLEWMGWVNPNSGNTGSTQKFQGRVTMTRNTSISTAYMELS | QKPGQAPRLLIYGSSTRATGIPARFRGSGSGTDFTLTI |
| | SLRSEDTAVYYCARGVLKGDYYYMDVWGKGTTVTVSS | SSLQPEDFAVYYCQQDDHLPLTFGGGTKVEIK |
| SCT1.48 | SEQ ID NO: 672 | SEQ ID NO: 671 |
| | QVQLVQSGAEVKKPGASVKVSCKASGYTFTSYDINWVRQATG | EIVMTQSPATLSLSPGERATLSCRASQSISSTYLSWYQ |
| | QGLEWMGWVNPNSGNTGSTQKFQGRVTMTRNTSISTAYMELS | QKPGQAPRLLIYGSSTRATGIPARFSGSGSGTDETLTI |
| | SLRSEDTAVYYCARGVLKGDYYYMDVWGKGTTVTVSS | SSLQPEDFAVYYCQQDDHLPLTFGGGTKVEIK |
| keliximab | SEQ ID NO: 674 | SEQ ID NO: 675 |
| | MKHLWFFLLLVAAPRWVLSQVQLQEAGPGLVKPSETLSLTCS | MAWALLLLGLLAHFTDSAASYELSQPRSVSVSPGQTAG |
| | VSGGSISGDYYWFWIRQSPGKGLEWIGYIYGSGGGTNYNPSL | FTCGGDNVGRKSVQWYQQKPPQAPVLVIYADSERPSGI |
| | NNRVSISIDTSKNLFSLKLRSVTAADTAVYYCASNILKYLHW | PARFSGSNSGNTATLTISGVEAGDEADYYCQVWDSTAD |
| | LLYWGQGVLVTVS | HWVFGGGTRLTVLG |
| UB-421 | SEQ ID NO: 676 | SEQ ID NO: 677 |
| | QVQLVQSGPELKKPGASVKVSCKASGYTFTDYVIHWVKQATG | DIVLTQSPASLAVSLGQRATITCKAGQSVDYDGDSYMN |
| | QGLEWIGEIYPGSGSAYSNAKFKDRVTMTADKSSNTAYMELS | WYQQKPGQPPKLLIYVASNLESGIPARFSGSGSGTDFT |
| | SLTSDDTAVYFCARRGNGTGFAYWGQGTLVTVSS | LNIHPVEENDAATYYCQQSYKDPLTFGQGTKLEIK |

Binds to CD4 D2

| | | |
|---|---|---|
| ibalizumab | SEQ ID NO: 678 | SEQ ID NO: 679 |
| | QVQLQQSGPEVVKPGASVKMSCKASGYTFTSYVIHWVRQKPG | DIVMTQSPDSLAVSLGERVTMNCKSSQSLLYSTNQKNY |
| | QGLDWIGYINPYNDGTDYDEKFKGKATLTSDTSTSTAYMELS | LAWYQQKPGOSPKLLIYWASTRESGVPDRFSGSGSGTD |
| | SLRSEDTAVYYCAREKDNYATGAWFAYWGQGTLVTVSS | FTLTISSVQAEDVAVYYCQQYYSYRTFGGGTKLEIK |
| tregalizumab | SEQ ID NO: 680 | SEQ ID NO: 681 |
| | EEQLVESGGGLVKPGGSLRLSCAASGFSFSDCRMYWLRQAPG | DIVMTQSPDSLAVSLGERATINCRASKSVSTSGYSYIY |
| | KGLEWIGVISVKSENYGANYAESVRGRFTISRDDSKNTVYLQ | WYQQKPGOPPKLLIYLASILESGVPDRESGSGSGTDET |
| | MNSLKTEDTAVYYCSASYYRYDVGAWFAYWGQGTLVTVSS | LTISSLQAEDVAVYYCQHSRELPWTFGQGTKVEIK |
| SCT1.1 | SEQ ID NO: 682 | SEQ ID NO: 683 |
| | QVQLVQSGAEVKKPGASVKVSCKVSGYTLTELSMHWVRQAPE | DIVMTQTPLSLSVTPGQPASISCKSSQSLLHTDGKTYL |
| | KGLEWMGGFDPEDGKTIYAPKFQGRVTMTEDTSTDTAYMDLS | YWYLQKPGQPPHLLIYEVSNRFSGVPDRLSGSGSGTDF |
| | SLRSEDTAVYYCATGHNWNDGYYFYYYMDLWGKGTTVTVSS | TLKISRVEAEDVGVYYCMQSLQLPLTFGGGTMVEIK |
| SCT1.4 | SEQ ID NO: 684 | SEQ ID NO: 685 |
| | QVQLVESGGDSVKPGGSLRLSCAASGFTFSDYYMNWIRQAPG | EIVMTQSPATLSVSPGERATLSCRASQSVSRKLAWYQQ |
| | KGLEWISYISSSGNTIFYVDSVEGRFTVSRDNAKNSLYLQMN | KPGQAPRLLIYAASTRATGIPARFSGSGSGTEFTLTIS |
| | SLRAEDTAVYYCAREGYNNYNYSYYYFMDVWGKGTTVTVSS | SLQSEDFAVYFCQQYNNWPYTFGRGTKLEIK |
| SCT1.5 | SEQ ID NO: 686 | SEQ ID NO: 687 |
| | QVQLVESGGGVVQPGRSLRLSCAASGFTVSSFGMHWVRQAPG | AIQMTQSPSSLSASVGDRVTFTCRASQGIRSGLGWYQQ |
| | KGLEWVAIIWYDGTNKYYADSVKGRFTISRDNSKNTLYLQLN | KPGKAPNLLIYAASTLQSGVPSRESGSGSGTDFTLTIT |
| | SLRGEDTAVYYCAREIAVDGTDYYMDVWGRGTTVTVSS | SLQPEDFATYYCLQDYNYPYTFGQGTKLEIK |
| SCT2.9 | SEQ ID NO: 688 | SEQ ID NO: 689 |
| | QVQLVESGGGLVTPGGSLRLSCAASGFTFSDYYMNWIRQAPG | EIVMTQSPATLSVSPGERATLSCRASQSVSSNLAWYQQ |
| | KGLEWLSYISSSGLTIFYVDSVKGRFTVSRDNAKNSLYLQMN | KPGQAPRLLIYGASIRATGIPARFSGSGSGTEFTLTIS |
| | SLRAEDTAVYYCAREGYSGFDDHYYYYTDVWGKGTTVTVSS | SLQSEDFAVYYCQHYKNWPYTFGQGTRLEIK |
| SCT2.10 | SEQ ID NO: 690 | SEQ ID NO: 691 |
| | QVQLVESGGGLVKPGGSLRLSCAASGFTESDYYMNWIRQAPG | EIVMTQSPAALSVSPGERATLSCRASQSVSSKFAWYQQ |
| | KGLEWVSYISSSGSTIFYTDSVKGRFTISRDNAKNSLYLQMN | KPGQAPRLLIYNASTRATGIPARFSGSGSGTEFTLTIS |
| | SLRAEDTAVYYCAREDYSNYEDYYYYYTGVWGKGTTVTVSS | SLQSEDFAVYYCQQYNNWPYTFGQGTKLEIK |
| SCT2.11 | SEQ ID NO: 692 | SEQ ID NO: 693 |
| | QVQLVESGGGLVKPGGSLRLSCSASGFTFSDYYMNWIRQAPG | EIVMTQSPAALSVSPGERATLSCRASQSVSNKFAWYQQ |
| | KGLEWVSYISSSGITIFYTDSVKGRFTISRDNAKNSLYLQMN | KPGOAPRLLIYNASTRATSIPARFSGSGSGTEFTLTIS |
| | SLRAEDTAVYYCAREDYSNYEDHYYYYTGVWGKGTTVTVSS | SLQSEDFAVYYCQHYNNWPYTFGQGTKLEIK |
| SCT2.11 | SEQ ID NO: 692 | SEQ ID NO: 694 |
| VL N50G | QVQLVESGGGLVKPGGSLRLSCSASGFTFSDYYMNWIRQAPG | EIVMTQSPAALSVSPGERATLSCRASQSVSNKFAWYQQ |
| | KGLEWVSYISSSGITIFYTDSVKGRFTISRDNAKNSLYLQMN | KPGQAPRLLIYGASTRATSIPARFSGSGSGTEFTLTIS |
| | SLRAEDTAVYYCAREDYSNYEDHYYYYTGVWGKGTTVTVSS | SLQSEDFAVYYCQHYNNWPYTFGQGTKLEIK |
| SCT2.12 | SEQ ID NO: 695 | SEQ ID NO: 696 |
| | QVQLVESGGGLVKPGGSLRLSCAASGFTESDYYMNWIRQAPG | EIVMTQSPAALSVSPGERATLSCRASQSVSSKFAWYQQ |
| | KGLEWVSYISSSGKTIFYTDSVKGRFTISRDNAKNSLFLQMN | KPGQAPRLLIYNASTRATGIPARFSGSGSGTEFTLTIS |
| | SLRAEDTAVYYCAREDYSNYEDYYYYYTGVWGKGTTVTVSS | SLQSEDFAVYYCHQYKNWPYTFGQGTKLEIK |
| SCT2.12 | SEQ ID NO: 695 | SEQ ID NO: 697 |
| VL N50G | QVQLVESGGGLVKPGGSLRLSCAASGFTFSDYYMNWIRQAPG | EIVMTQSPAALSVSPGERATLSCRASQSVSSKFAWYQQ |
| | KGLEWVSYISSSGKTIFYTDSVKGRFTISRDNAKNSLFLQMN | KPGQAPRLLIYGASTRATGIPARFSGSGSGTEFTLTIS |
| | SLRAEDTAVYYCAREDYSNYEDYYYYYTGVWGKGTTVTVSS | SLQSEDFAVYYCHQYKNWPYTFGQGTKLEIK |
| SCT2.13 | SEQ ID NO: 698 | SEQ ID NO: 699 |
| | QVQLVESGGGLVKPGGSLRLSCAASGFTFSDYYMNWIRQAPG | EILMTQSPATLSVSPGERTTLSCRASQSISSNLAWYQQ |
| | KGLEWISYISISGQTIYYGDSVKGRFTVSRDNAKNSLFLEMN | NPGQAPRLLIYNASTRATDIPARFSGGGSGTEFTLTIS |
| | SLRAEDSAVYFCAREGYSNYGVKYYYYMDVWGKGTTVTVSS | SLQSEDFAVYYCQQYNAWTYTFGQGTKLEIK |
| SCT2.14 | SEQ ID NO:700 | SEQ ID NO: 701 |
| | QVQLVESGGGLVKPGGSLRLSCAASGFTFSDHYMNWVRQVPG | EIMMTQSPATLSVSPGERATLSCRASQSVSSNLAWYQQ |
| | KGLEWISYISISGNTIYYTDSVKGRFTVSRDNAKNSLYLQMT | KPGQAPRLLIYGASTRATGIPARFSGSGSGTEFTLTIS |
| | SLRAEDTAVYYCVREGYSSSSRGDYSYYTDVWGKGTTVTVSS | SLQSEDFAVYYCQQYKNWPYTFGQGTKLEIK |
| SCT2.15 | SEQ ID NO: 702 | SEQ ID NO:703 |
| | QVQLVESGGGLVKPGGSLRLSCAASGFTFSDYYMNWVRQVPG | EIVMTQSPATLSASPGERVNLSCRASQGVSSNLAWYQQ |
| | KGLEWISYISISGRTIYYIDSVKGRFTVSRDNAKNSLYLQMT | KVGQAPRLLIYGASTRATDIPARESGSGSGSEFTLTIS |
| | NLRAEDTAVYYCVREGYSSSSRGDYSYYTDVWGKGTTVTVSS | SLQSEDFAVYYCQHYKNWPYTFGQGTKLEIK |

Binds to CD4 D2 and/or D3

| | | |
|---|---|---|
| SCT1.6 | SEQ ID NO: 704 | SEQ ID NO: 705 |
| | QVQLVESGGGVVQPGRSLRLSCAASGFTVSSYGMHWVRQAPG | AIQMTQSPSSLSASVGDRVTITCRASQDIRNGLGWYQQ |
| | KGLEWVAIIWFDGSNKYYADSLTGLFTISRDSSKNTLFLQMN | KPGKAPKLLIYTTSSLQTGVPSRFSGSGSGTDFTLTIS |
| | SLSLEDTAVYYCAREVALEGYYYYMDVWGKGTTVTVSS | SLQPEDFATYYCLQDYNYPYTFGQGTKLEIK |
| SCT1.7 | SEQ ID NO: 706 | SEQ ID NO: 707 |
| | QVQLVESGGGVVQPGRSLSLSCAASGFTVSSYGMHWVRQAPG | AIQMTQSPSSLSASVGDRVTITCRASQDIRNGLGWYQQ |
| | KGLEWVAILWEDGSNKFYADSVKGRFTISRDNSKNTLYLQMN | NPGKAPKLLIYTTSSLQSGVPSRFSGSGSGTDFTLTIS |
| | SLRVEDTAVYYCARELALEGYYYYMDVWGKGTTVTVSS | SLQPEDFATYYCLQDYNYPYTFGQGTKLEIK |
| SCT1.8 | SEQ ID NO: 708 | SEQ ID NO:709 |
| | QVQLVESGGGVVQPGRSLRLSCAASGFTVSSYGMHWVRQAPG | AIQMTQSPSSLSASVGDRVTITCRSSQDIRNGLGWYQQ |
| | KGLEWVAILWEDGSNQFYADSVKGRFTISRDNSKNTLYLQMN | KPGKAPKLLIYTTSSLQSGVPSRESGSGSGTDFTLTIS |
| | SLRVEDTAVYYCAREIALEGYYYYMDVWGKGTTVTVSS | SLQPEDFATYYCLQDYNYPYTFGQGTKLEIK |
| SCT1.9 | SEQ ID NO: 710 | SEQ ID NO: 711 |
| | QVQLVESGGGVVQPGRALRLSCAASGFTFSSYGMHWVRQAPG | DIQMTQSPSSLSASVGDRVTITCRASQDIRNGLGWYQQ |
| | KGLEWVAIIWYDGSNKYYADSVKGRFTISRDNSKNTLYLQMN | KPGKAPKLLIYTTSNLQSGVPSRFSGSGSGTDETLTIS |
| | SLRAEDTAVYYCAREVALEGYYYYMDVWGKGTTVTVSS | SLQPEDFATYYCLQDYNYPYTFGQGTKLEIK |
| SCT1.10 | SEQ ID NO: 712 | SEQ ID NO: 713 |
| | QVQLVESGGGVVQPGRSLRLSCAASGFTFSSYGMHWVRQAPG | AIQMTQSPSSLSASVGDRVTITCRASQGIRNGLGWYQL |
| | KGLEWVAIIWYDGNNKYYADSVKGRFTISRDNSKNTLYLQMN | KPGKAPKLLIYAASSLQSGVPSSFSGSGSGTDFTLTIS |
| | SLRAEDTAVYYCTREVALEGYYYYMDVWGKGTTVTVSS | SLQPEDFATYYCLQDYNYPYTFGQGTKLEIK |
| SCT1.19 | SEQ ID NO: 714 | SEQ ID NO: 715 |
| | EVQLLESGGGLVQPGGSLRLSCAASGFTFSSYAMSWVRQAPG | AIQMTQSPSSLSAFVGDRVTITCRASQGIRNDLGWYQQ |
| | KGLEWVSGISGSVGSTYYADSVKGRFTISRDKSKNTLYLQMN | KPGKAPKLLIYAASSLQSGVPSRFSGSGSGTDFTLTIS |
| | SLRAEDTAVYYCAKEGYDWSGMDVWGKGTTVTVSS | SLQPEDFATYYCLQDYNYPYTFGQGTKLEIK |
| SCT2.2 | SEQ ID NO: 716 | SEQ ID NO: 717 |
| | QVLLVESGGGVVQPGRSLRLSCAASGFTFSSYGMHWVRQAPG | AIQMTQSPSSLSASVGDRVTITCRASQGIRNGLGWYQQ |
| | KGLEWVTLIWFDGTTRFYADSVKGRFTVSRDNSKKTLYLQMN | KPGKAPKLLIYAASSLQSGVPSRFSGSGSGTDFTLTIS |
| | SLRAEDTAVYYCAREVAIQGRDYYIDVWGKGTTVTVSS | SLQPADFATYYCLQDYNYPYTFGQGTKLEIR |
| SCT2.3 | SEQ ID NO: 718 | SEQ ID NO: 719 |
| | QVQLVESGGGVVQPGRSLRLSCAASGFTFSSYGIHWFRQAPG | AIQMTQSPSSLSASVGDRVTITCRASQDIRNGLGWYQQ |
| | KGLEWVAIIWFDGTNKFYADSVKGRFTISRDNSKNTVNLQMN | KPGKAPMLLIYTTSNLQNGVPSRESGSRSGTDFTLTIS |
| | SLRVEDTAIYYCAREVAIQGYYYYMDVWGNGTTVTVSS | GLQPEDFAAYFCLQDYNYPYTFGQGTKLDVK |

Binds to CD4 D3

| | | |
|---|---|---|
| OKT4 | SEQ ID NO: 720 | SEQ ID NO: 721 |
| | QIQLVQSGPELKKPGETVKISCKASGYTFTNYGMNWVKQAPG | NIVLTQSPASLAVSLGQRATISCRASESVDSYGNSFMH |
| | KGLKCMGWINTNTGEPTYAEEFKGRFAFSLETSATTAFLQIN | WYQQKPGQPPKLFIYLASNLESGVPARFSGSGSRTDFT |
| | NLKDEDTATYFCARLGIYYDYGYYAMDYWGQGASVTVSS | LTIDPVEADDAATYYCQQNNEDPYTFGGGTKLEIK |
| SCT1.20 | SEQ ID NO: 722 | SEQ ID NO: 723 |
| | EVQLLESGGGLVQPGGSLRLSCAASEIIFSTYAMSWVRQAPG | AIQMTQSPYSLSASVGDRVTITCRASQGIRNDLGWYQQ |
| | KGLEWVSGISGSGDNTYYADSVKGRFTISSDNSKNTLYLQMN | KPGKAPKVLIYEASSLQSGVPSRFSGSRSGTDFTLTIS |
| | SLRAEDTAVYYCVKEGYNWNYMDVWGKGTTVTVSS | SLQPEDFATYYCLQDYTYPYTFGQGTKLEIK |
| SCT1.21 | SEQ ID NO: 724 | SEQ ID NO: 725 |
| | EVELLESGGGLVQPGGSLRLSCAASGLTESTFAMSWVRQAPG | AIQMTQSPSSLSASVGDRVTITCRASHGIRNDLGWYQQ |
| | KGLEWVSGISGSGENTYYADSVKGRFTITSDNSKNTLYLQMN | KPGKAPKVLISETSSLQSGVPSRFSGSRSGTDFTLTIS |
| | SLRAEDTAVYYCAKEGYNWNYMDVWGKGTTVTVSS | SLQPEDFATYYCLQDYTYPYTFGQGTKLEIK |
| SCT1.22 | SEQ ID NO: 724 | SEQ ID NO: 726 |
| | EVELLESGGGLVQPGGSLRLSCAASGLTESTFAMSWVROAPG | AIQMTQSPSSLSASVGDRVTITCRASHGIRNDLGWYQQ |
| | KGLEWVSGISGSGENTYYADSVKGRFTITSDNSKNTLYLQMN | KPGKAPKVLIYEASSLQSGVPSRFSGSRSGTDFTLTIS |
| | SLRAEDTAVYYCAKEGYNWNYMDVWGKGTTVTVSS | SLQPEDFATYYCLQDYTYPYTFGQGTKLEIK |
| SCT1.23 | SEQ ID NO: 727 | SEQ ID NO: 728 |
| | EVQLLESGGGLVQPGGSLRLSCAASGFTESTYAMSWVRQAPG | AIQMTQSPSSLSASVGDRVTITCRASQGIRNDLGWCQQ |
| | KGLEWVSTISDNIGNTYYADSVKGRFTISRDNSKNTLYLQMN | KPGKAPKFLIYAASTLQSGVPSRFSGSGYGTDFTLTIS |
| | SLRAEDTAVYYCTKDNEDYYMDVWGKGTTVTVSS | SLQPEDFATYYCLQDYNYPRTFGQGTKVEIK |
| SCT1.23 | SEQ ID NO: 727 | SEQ ID NO: 729 |
| C36Y | EVQLLESGGGLVQPGGSLRLSCAASGFTFSTYAMSWVRQAPG | AIQMTQSPSSLSASVGDRVTITCRASQGIRNDLGWYQQ |
| | KGLEWVSTISDNIGNTYYADSVKGRFTISRDNSKNTLYLQMN | KPGKAPKFLIYAASTLQSGVPSRESGSGYGTDFTLTIS |
| | SLRAEDTAVYYCTKDNEDYYMDVWGKGTTVTVSS | SLQPEDFATYYCLQDYNYPRTFGQGTKVEIK |
| SCT2.1 | SEQ ID NO:730 | SEQ ID NO: 731 |
| | EVQLLESGGGLVQPGGSLRLSCAASGFTESTNAMSWVRQAPG | AIQMTQSPSSLSASVGDRVTITCRASHVIRNDLGWYQQ |
| | KGLEWVSGISGSNGNTYYADSVKGRFIISRDISKNTLYLEMN | KPGKAPKLLIYAASSLQSGVPSRFSGSGSGTDETLTIS |
| | SLRAEDTAVYYCAKERDNWNGFDYWGQGTLVTVSS | SLQPEDFATYYCLQDYNYPYTFGQGTKLEIK |
| SCT2.8 | SEQ ID NO: 732 | SEQ ID NO:733 |
| | QVQLVQSGAEVKKPGASVKVSCKVSGHTLTELSMHWVRQAPG | ETTLTQSPAFMSATPGDKVNISCKASQDIDDDVNWYQQ |
| | KGLEWMGIFDPRDGQIIYAEKFQGRVTVTEDTSTDTAYMELS | KPGEAAIFIIQEATTLVPGIPPRESGSGYGTDETLTIN |
| | SLRFDDTAVYYCATGGNEDYYFYYMDVWGKGTTVTVSS | NIESEDAAYYFCLQHDHFPYTFGQGTKLEIK |

### 3. Interleukin-15 (IL-15) Variants and Fusion Polypeptides Thereof

Provided are interleukin-15 (IL-15) variants (IL-15v) with attenuated binding to IL-2Rβγ (CD122, NCBI Gene ID: 3560; CD132, NCBI Gene ID: 3561), e.g., in comparison to wild-type IL-15 (SEQ ID NO: 740). In some embodiments, the IL-15v binds to IL-2Rβ with a binding equilibrium dissociation constant (K_{D}) of at least 1 μM, e.g., a K_{D} of at least 1.5 μM, at least 2 μM, at least 2.5 μM, at least 3 μM, at least 3.5 μM, at least 4 μM, at least 4.5 μM or at least 5 μM. In some embodiments, the IL-15v binds to IL-2Rβγ with a binding equilibrium dissociation constant (K_{D}) of at least 1 μM, e.g., a K_{D} of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM. In some embodiments, the IL-15v described herein induce CD4+ T cell proliferation with an EC50 of less than 5 nM, e.g., less than 4 nM, less than 3 nM, less than 2 nM, less than 1 nM, e.g., wherein CD4+ T cell proliferation potency is measured by marker of proliferation Ki-67 activation (MK167; NCBI Gene ID: 4288). In some embodiments, the IL-15v induce CD8+ T cells and/or natural killer (NK) cell proliferation with an EC50 of greater than 100 nM, e.g., wherein CD8+ T cell and/or NK cell proliferation potency is measured by Ki-67 activation. In some embodiments, the IL-15v described herein induce CD4+ T cell proliferation with a potency that is at least 100-fold, e.g., at least 200-fold, at least 300-fold, at least 400-fold, at least 500-fold, at least 600-fold, or more, in comparison to the potency for inducing CD8+ T cell or NK cell proliferation, e.g., wherein cell proliferation potency is measured by Ki-67 activation.

A polypeptide “variant,” as the term is used herein, is a polypeptide that typically differs from a polypeptide specifically disclosed herein in one or more substitutions, deletions, additions and insertions. Such variants may be naturally occurring or may be synthetically generated, for example, by modifying one or more of the above polypeptide sequences described herein and evaluating one or more biological activities of the polypeptide as described herein, e.g., using any of a number of techniques well known in the art.

The term “variant” may also refer to any naturally occurring or engineered molecule comprising one or more nucleotide mutations/substitutions or amino acid substitutions. For example, somatic variants may encompass all related naturally occurring antibodies that are part of or derived from the same B-cell lineage. Engineered variants may encompass all single substitutions or combinatorial substitutions made to an antibody.

a. IL-15v

In some embodiments, provided is a human interleukin-15 variant (IL-15v) comprising the following amino acid substitutions with respect to human wild-type IL-15 (NCBI Gene ID: 3600; Uniprot P40933): a glycine (Gly; G) at position 7 (S7G); an alanine (Ala; A) at position 68 (168A); a leucine (Leu; L) or a glutamic acid (Glu; E) at position 71 (N71L or N71E); a leucine (Leu; L) at position 77 (N77L); a glycine (Gly; G) or a proline (Pro; P) at position 79 (N79G or N79P); a lysine (Lys; K) at position 112 (N112K); or one of the following combinations of amino acid substitutions: N1D, N4D and D8N; N1D and D61N; N1D and E64Q; N1D and N65D; N4D and D61N; N4D and E64Q; N4D and N65D; S7G and N65D; D8G and D61N; D8G and E64Q; D8G and N65D; D8N and D61N; D8N and E64Q D8N and N65D; D30N and N65D; D61N and E64Q; D61N and N65D; E64Q and N65D; E64Q and Q108E; N65A and 168A; N65D and 168A; N71L and N79P; N71L and N112D; N79P and N112D; S7G, N65A and 168A; S7G, N65D and 168A; D30N, E64Q and N65D; D61N, E64Q and N65D; D61N, N65D and 168A; N71Q, N79Q and N112Q; N71A, N79A and N112A; N71G, N79G and N112G; N71S, N79L and N112E; N71E, N79P and N112K; N71L, N79P and N112K; N71L, N79P and N112D; N71K, N79P and N112D; N71E, N79P and N112S; N71E, N79P and N112D; N1D, D61N, E64Q and Q108E; N4D, D61N, E64Q and Q108E; N71Q, N77L, N79Q and N112Q; N71A, N77L, N79A and N112A; N71G, N77L, N79G and N112G; N71S, N77L, N79L and N112E; N71E, N77L, N79P and N112K; N71L, N77L, N79P and N112K; N71E, N77L, N79P and N112S; N71E, N77L, N79P and N112D; S7G, N65D, N71L, N79P and N112D; S7G, N65D, N71E, N77L, N79P and N112D; S7G, N65D, N71E, N77L, N79P and N112S; S7G, N65D, N71L, N77L, N79P and N112K; or S7G, N65D, N71E, N77L, N79P and N112K; wherein the position numbers are with respect to SEQ ID NO: 740 (wild-type IL-15).

In some embodiments, provided is an interleukin-15 variant (IL-15v) comprising an amino acid sequence of any one of SEQ ID NOs: 743, 750-752, 754-756, 758, 768-771, 774, 777, 779, 783-788, 790-798, 801-808, 810-813 and 1125. In some embodiments, provided is an interleukin-15 variant (IL-15v) comprising an amino acid sequence of SEQ ID NO: 743. In some embodiments, provided is an interleukin-15 variant (IL-15v) comprising an amino acid sequence of SEQ ID NO: 750. In some embodiments, provided is an interleukin-15 variant (IL-15v) comprising an amino acid sequence of SEQ ID NO: 751. In some embodiments, provided is an interleukin-15 variant (IL-15v) comprising an amino acid sequence of SEQ ID NO: 752. In some embodiments, provided is an interleukin-15 variant (IL-15v) comprising an amino acid sequence of SEQ ID NO: 754. In some embodiments, provided is an interleukin-15 variant (IL-15v) comprising an amino acid sequence of SEQ ID NO: 755. In some embodiments, provided is an interleukin-15 variant (IL-15v) comprising an amino acid sequence of SEQ ID NO: 756. In some embodiments, provided is an interleukin-15 variant (IL-15v) comprising an amino acid sequence of SEQ ID NO: 758. In some embodiments, provided is an interleukin-15 variant (IL-15v) comprising an amino acid sequence of SEQ ID NO: 768. In some embodiments, provided is an interleukin-15 variant (IL-15v) comprising an amino acid sequence of SEQ ID NO: 769. In some embodiments, provided is an interleukin-15 variant (IL-15v) comprising an amino acid sequence of SEQ ID NO: 770. In some embodiments, provided is an interleukin-15 variant (IL-15v) comprising an amino acid sequence of SEQ ID NO: 771. In some embodiments, provided is an interleukin-15 variant (IL-15v) comprising an amino acid sequence of SEQ ID NO: 774. In some embodiments, provided is an interleukin-15 variant (IL-15v) comprising an amino acid sequence of SEQ ID NO: 777. In some embodiments, provided is an interleukin-15 variant (IL-15v) comprising an amino acid sequence of SEQ ID NO: 779. In some embodiments, provided is an interleukin-15 variant (IL-15v) comprising an amino acid sequence of SEQ ID NO: 783. In some embodiments, provided is an interleukin-15 variant (IL-15v) comprising an amino acid sequence of SEQ ID NO: 784. In some embodiments, provided is an interleukin-15 variant (IL-15v) comprising an amino acid sequence of SEQ ID NO: 785. In some embodiments, provided is an interleukin-15 variant (IL-15v) comprising an amino acid sequence of SEQ ID NO: 786. In some embodiments, provided is an interleukin-15 variant (IL-15v) comprising an amino acid sequence of SEQ ID NO: 787. In some embodiments, provided is an interleukin-15 variant (IL-15v) comprising an amino acid sequence of SEQ ID NO: 788. In some embodiments, provided is an interleukin-15 variant (IL-15v) comprising an amino acid sequence of SEQ ID NO: 790. In some embodiments, provided is an interleukin-15 variant (IL-15v) comprising an amino acid sequence of SEQ ID NO: 791. In some embodiments, provided is an interleukin-15 variant (IL-15v) comprising an amino acid sequence of SEQ ID NO: 792. In some embodiments, provided is an interleukin-15 variant (IL-15v) comprising an amino acid sequence of SEQ ID NO: 793. In some embodiments, provided is an interleukin-15 variant (IL-15v) comprising an amino acid sequence of SEQ ID NO: 794. In some embodiments, provided is an interleukin-15 variant (IL-15v) comprising an amino acid sequence of SEQ ID NO: 795. In some embodiments, provided is an interleukin-15 variant (IL-15v) comprising an amino acid sequence of SEQ ID NO: 796. In some embodiments, provided is an interleukin-15 variant (IL-15v) comprising an amino acid sequence of SEQ ID NO: 797. In some embodiments, provided is an interleukin-15 variant (IL-15v) comprising an amino acid sequence of SEQ ID NO: 798. In some embodiments, provided is an interleukin-15 variant (IL-15v) comprising an amino acid sequence of SEQ ID NO: 799. In some embodiments, provided is an interleukin-15 variant (IL-15v) comprising an amino acid sequence of SEQ ID NO: 801. In some embodiments, provided is an interleukin-15 variant (IL-15v) comprising an amino acid sequence of SEQ ID NO: 802. In some embodiments, provided is an interleukin-15 variant (IL-15v) comprising an amino acid sequence of SEQ ID NO: 803. In some embodiments, provided is an interleukin-15 variant (IL-15v) comprising an amino acid sequence of SEQ ID NO: 804. In some embodiments, provided is an interleukin-15 variant (IL-15v) comprising an amino acid sequence of SEQ ID NO: 805. In some embodiments, provided is an interleukin-15 variant (IL-15v) comprising an amino acid sequence of SEQ ID NO: 806. In some embodiments, provided is an interleukin-15 variant (IL-15v) comprising an amino acid sequence of SEQ ID NO: 807. In some embodiments, provided is an interleukin-15 variant (IL-15v) comprising an amino acid sequence of SEQ ID NO: 808. In some embodiments, provided is an interleukin-15 variant (IL-15v) comprising an amino acid sequence of SEQ ID NO: 810. In some embodiments, provided is an interleukin-15 variant (IL-15v) comprising an amino acid sequence of SEQ ID NO: 811. In some embodiments, provided is an interleukin-15 variant (IL-15v) comprising an amino acid sequence of SEQ ID NO: 812. In some embodiments, provided is an interleukin-15 variant (IL-15v) comprising an amino acid sequence of SEQ ID NO: 813. In some embodiments, provided is an interleukin-15 variant (IL-15v) comprising an amino acid sequence of SEQ ID NO: 1125.

Illustrative IL-15v are provided in Table D, below. The IL-15v provided in Table D do not have any non-naturally occurring cysteine residues.

b. IL-15RA Sushi Domain-IL-15v Fusion Polypeptides

In some embodiments, provided is a fusion protein comprising: (i) an IL-15 receptor alpha subunit (IL15RA) SUSHI domain and (ii) an IL-15 variant (IL-15v), the fusion protein comprising an amino acid sequence of any one of SEQ ID NOs: 817, 824-826, 828-830, 832, 842-845, 848, 851, 853, 857-862, 864-872, 875-882, 884-887 and 1127, as provided in Table E. In some embodiments, provided is a fusion protein comprising: (i) an IL-15 receptor alpha subunit (IL15RA) SUSHI domain and (ii) an IL-15 variant (IL-15v), the fusion protein comprising an amino acid sequence of any one of SEQ ID NOs: 1136, 1143-1145, 1147-1149, 1151, 1162-1165, 1168, 1171, 1173-1174, 1178-1183, 1185-1193 and 1196-1203, 1205-1208, as provided in Table E. In some embodiments, the IL15RA SUSHI domain is N-terminal to the IL-15v. In some embodiments, the IL15RA SUSHI domain binds to IL-15, is no longer than 65 amino acids, and comprises the amino acid sequence of SEQ ID NO: 734, or an amino acid sequence at least 90%, 91%, 92%, 93%, 94%, 95%, 96%, 97%, 98%, 99% identical to SEQ ID NO: 734. In some embodiments, the IL15RA SUSHI domain comprises the amino acid sequence of SEQ ID NO: 735, or an amino acid sequence at least 90%, 91%, 92%, 93%, 94%, 95%, 96%, 97%, 98%, 99% identical to SEQ ID NO: 735. Illustrative IL15RA SUSHI domain sequences are provided in Table C. below. In some embodiments, the IL15RA SUSHI domain comprises an amino acid sequence selected from SEQ ID NOs: 734-739. In some embodiments, the IL15RA SUSHI domain comprises an amino acid sequence of SEQ ID NO: 734. In some embodiments, the IL15RA SUSHI domain comprises an amino acid sequence of SEQ ID NO: 735. In some embodiments, the IL15RA SUSHI domain comprises an amino acid sequence of SEQ ID NO: 736. In some embodiments, the IL15RA SUSHI domain comprises an amino acid sequence of SEQ ID NO: 737. In some embodiments, the IL15RA SUSHI domain comprises an amino acid sequence of SEQ ID NO: 738. In some embodiments, the IL15RA SUSHI domain comprises an amino acid sequence of SEQ ID NO: 739.

In some embodiments, the IL15RA SUSHI domain and IL-15v are connected via a flexible linker, e.g., a Gly-Ser linker. In some embodiments, the linker has a length of from about 4 to about 50 amino acids, e.g., from about 5 amino acids to about 25 amino acids, e.g., from about 15 amino acids to about 25 amino acids. In some embodiments, the linker comprises from 1 to 10 units, e.g., 1 to 5 units, e.g., 3 to 5 units, of a poly-glycine serine linker selected from GGGS (SEQ ID NO: 1128), GGGGS (SEQ ID NO: 1129) and combinations thereof. In some embodiments, the linker comprises 5 units of GGGGS (SEQ ID NO: 1129) (GGGGSGGGGSGGGGSGGGGSGGGGS; SEQ ID NO: 1085).

In some embodiments, the fusion protein comprising an IL15RA SUSHI domain and an IL-15v comprises an amino acid sequence of any one of SEQ ID NOs: 814-887, 1126-1127. In some embodiments, the fusion protein comprising an IL15RA SUSHI domain and an IL-15v comprises an amino acid sequence of any one of SEQ ID NOs: 817, 824-826, 828-830, 832, 842-845, 848, 851, 853, 857-862, 864-872, 875-882, 884-887 and 1127. In some embodiments, the fusion protein comprising an IL15RA SUSHI domain and an IL-15v comprises an amino acid sequence of SEQ ID NO: 817. In some embodiments, the fusion protein comprising an IL15RA SUSHI domain and an IL-15v comprises an amino acid sequence of SEQ ID NO: 824. In some embodiments, the fusion protein comprising an IL15RA SUSHI domain and an IL-15v comprises an amino acid sequence of SEQ ID NO: 825. In some embodiments, the fusion protein comprising an IL15RA SUSHI domain and an IL-15v comprises an amino acid sequence of SEQ ID NO: 826. In some embodiments, the fusion protein comprising an IL15RA SUSHI domain and an IL-15v comprises an amino acid sequence of SEQ ID NO: 828. In some embodiments, the fusion protein comprising an IL15RA SUSHI domain and an IL-15v comprises an amino acid sequence of SEQ ID NO: 829. In some embodiments, the fusion protein comprising an IL15RA SUSHI domain and an IL-15v comprises an amino acid sequence of SEQ ID NO: 830. In some embodiments, the fusion protein comprising an IL15RA SUSHI domain and an IL-15v comprises an amino acid sequence of SEQ ID NO: 832. In some embodiments, the fusion protein comprising an IL15RA SUSHI domain and an IL-15v comprises an amino acid sequence of SEQ ID NO: 842. In some embodiments, the fusion protein comprising an IL15RA SUSHI domain and an IL-15v comprises an amino acid sequence of SEQ ID NO: 843. In some embodiments, the fusion protein comprising an IL15RA SUSHI domain and an IL-15v comprises an amino acid sequence of SEQ ID NO: 844. In some embodiments, the fusion protein comprising an IL15RA SUSHI domain and an IL-15v comprises an amino acid sequence of SEQ ID NO: 845. In some embodiments, the fusion protein comprising an IL15RA SUSHI domain and an IL-15v comprises an amino acid sequence of SEQ ID NO: 848. In some embodiments, the fusion protein comprising an IL15RA SUSHI domain and an IL-15v comprises an amino acid sequence of SEQ ID NO: 851. In some embodiments, the fusion protein comprising an IL15RA SUSHI domain and an IL-15v comprises an amino acid sequence of SEQ ID NO: 853. In some embodiments, the fusion protein comprising an IL15RA SUSHI domain and an IL-15v comprises an amino acid sequence of SEQ ID NO: 857. In some embodiments, the fusion protein comprising an IL15RA SUSHI domain and an IL-15v comprises an amino acid sequence of SEQ ID NO: 858. In some embodiments, the fusion protein comprising an IL15RA SUSHI domain and an IL-15v comprises an amino acid sequence of SEQ ID NO: 859. In some embodiments, the fusion protein comprising an IL15RA SUSHI domain and an IL-15v comprises an amino acid sequence of SEQ ID NO: 860. In some embodiments, the fusion protein comprising an IL15RA SUSHI domain and an IL-15v comprises an amino acid sequence of SEQ ID NO: 861. In some embodiments, the fusion protein comprising an IL15RA SUSHI domain and an IL-15v comprises an amino acid sequence of SEQ ID NO: 862. In some embodiments, the fusion protein comprising an IL15RA SUSHI domain and an IL-15v comprises an amino acid sequence of SEQ ID NO: 864. In some embodiments, the fusion protein comprising an IL15RA SUSHI domain and an IL-15v comprises an amino acid sequence of SEQ ID NO: 865. In some embodiments, the fusion protein comprising an IL15RA SUSHI domain and an IL-15v comprises an amino acid sequence of SEQ ID NO: 866. In some embodiments, the fusion protein comprising an IL15RA SUSHI domain and an IL-15v comprises an amino acid sequence of SEQ ID NO: 867. In some embodiments, the fusion protein comprising an IL15RA SUSHI domain and an IL-15v comprises an amino acid sequence of SEQ ID NO: 868. In some embodiments, the fusion protein comprising an IL15RA SUSHI domain and an IL-15v comprises an amino acid sequence of SEQ ID NO: 869. In some embodiments, the fusion protein comprising an IL15RA SUSHI domain and an IL-15v comprises an amino acid sequence of SEQ ID NO: 870. In some embodiments, the fusion protein comprising an IL15RA SUSHI domain and an IL-15v comprises an amino acid sequence of SEQ ID NO: 871. In some embodiments, the fusion protein comprising an IL15RA SUSHI domain and an IL-15v comprises an amino acid sequence of SEQ ID NO: 872. In some embodiments, the fusion protein comprising an IL15RA SUSHI domain and an IL-15v comprises an amino acid sequence of SEQ ID NO: 875. In some embodiments, the fusion protein comprising an IL15RA SUSHI domain and an IL-15v comprises an amino acid sequence of SEQ ID NO: 876. In some embodiments, the fusion protein comprising an IL15RA SUSHI domain and an IL-15v comprises an amino acid sequence of SEQ ID NO: 877. In some embodiments, the fusion protein comprising an IL15RA SUSHI domain and an IL-15v comprises an amino acid sequence of SEQ ID NO: 878. In some embodiments, the fusion protein comprising an IL15RA SUSHI domain and an IL-15v comprises an amino acid sequence of SEQ ID NO: 879. In some embodiments, the fusion protein comprising an IL15RA SUSHI domain and an IL-15v comprises an amino acid sequence of SEQ ID NO: 880. In some embodiments, the fusion protein comprising an IL15RA SUSHI domain and an IL-15v comprises an amino acid sequence of SEQ ID NO: 881. In some embodiments, the fusion protein comprising an IL15RA SUSHI domain and an IL-15v comprises an amino acid sequence of SEQ ID NO: 882. In some embodiments, the fusion protein comprising an IL15RA SUSHI domain and an IL-15v comprises an amino acid sequence of SEQ ID NO: 884. In some embodiments, the fusion protein comprising an IL15RA SUSHI domain and an IL-15v comprises an amino acid sequence of SEQ ID NO: 885. In some embodiments, the fusion protein comprising an IL15RA SUSHI domain and an IL-15v comprises an amino acid sequence of SEQ ID NO: 886. In some embodiments, the fusion protein comprising an IL15RA SUSHI domain and an IL-15v comprises an amino acid sequence of SEQ ID NO: 887. In some embodiments, the fusion protein comprising an IL15RA SUSHI domain and an IL-15v comprises an amino acid sequence of SEQ ID NO: 1126. In some embodiments, the fusion protein comprising an IL15RA SUSHI domain and an IL-15v comprises an amino acid sequence of SEQ ID NO: 1127.

In some embodiments, the fusion protein comprising an IL15RA SUSHI domain and an IL-15v comprises an amino acid sequence of any one of SEQ ID NOs: 1133-1208. In some embodiments, the fusion protein comprising an IL15RA SUSHI domain and an IL-15v comprises an amino acid sequence of any one of SEQ ID NOs: 1136, 1143-1145, 1147-1149, 1151, 1162-1165, 1168, 1171, 1173-1174, 1178-1183, 1185-1193 and 1196-1203 and 1205-1208. In some embodiments, the fusion protein comprising an IL15RA SUSHI domain and an IL-15v comprises an amino acid sequence of SEQ ID NO: 1133. In some embodiments, the fusion protein comprising an IL15RA SUSHI domain and an IL-15v comprises an amino acid sequence of SEQ ID NO: 1134. In some embodiments, the fusion protein comprising an IL15RA SUSHI domain and an IL-15v comprises an amino acid sequence of SEQ ID NO: 1135. In some embodiments, the fusion protein comprising an IL15RA SUSHI domain and an IL-15v comprises an amino acid sequence of SEQ ID NO: 1136. In some embodiments, the fusion protein comprising an IL15RA SUSHI domain and an IL-15v comprises an amino acid sequence of SEQ ID NO: 1137. In some embodiments, the fusion protein comprising an IL15RA SUSHI domain and an IL-15v comprises an amino acid sequence of SEQ ID NO: 1138. In some embodiments, the fusion protein comprising an IL15RA SUSHI domain and an IL-15v comprises an amino acid sequence of SEQ ID NO: 1139. In some embodiments, the fusion protein comprising an IL15RA SUSHI domain and an IL-15v comprises an amino acid sequence of SEQ ID NO: 1140. In some embodiments, the fusion protein comprising an IL15RA SUSHI domain and an IL-15v comprises an amino acid sequence of SEQ ID NO: 1141. In some embodiments, the fusion protein comprising an IL15RA SUSHI domain and an IL-15v comprises an amino acid sequence of SEQ ID NO: 1142. In some embodiments, the fusion protein comprising an IL15RA SUSHI domain and an IL-15v comprises an amino acid sequence of SEQ ID NO: 1143. In some embodiments, the fusion protein comprising an IL15RA SUSHI domain and an IL-15v comprises an amino acid sequence of SEQ ID NO: 1144. In some embodiments, the fusion protein comprising an IL15RA SUSHI domain and an IL-15v comprises an amino acid sequence of SEQ ID NO: 1145. In some embodiments, the fusion protein comprising an IL15RA SUSHI domain and an IL-15v comprises an amino acid sequence of SEQ ID NO: 1146. In some embodiments, the fusion protein comprising an IL15RA SUSHI domain and an IL-15v comprises an amino acid sequence of SEQ ID NO: 1147. In some embodiments, the fusion protein comprising an IL15RA SUSHI domain and an IL-15v comprises an amino acid sequence of SEQ ID NO: 1148. In some embodiments, the fusion protein comprising an IL15RA SUSHI domain and an IL-15v comprises an amino acid sequence of SEQ ID NO: 1149. In some embodiments, the fusion protein comprising an IL15RA SUSHI domain and an IL-15v comprises an amino acid sequence of SEQ ID NO: 1150. In some embodiments, the fusion protein comprising an IL15RA SUSHI domain and an IL-15v comprises an amino acid sequence of SEQ ID NO: 1151. In some embodiments, the fusion protein comprising an IL15RA SUSHI domain and an IL-15v comprises an amino acid sequence of SEQ ID NO: 1152. In some embodiments, the fusion protein comprising an IL15RA SUSHI domain and an IL-15v comprises an amino acid sequence of SEQ ID NO: 1153. In some embodiments, the fusion protein comprising an IL15RA SUSHI domain and an IL-15v comprises an amino acid sequence of SEQ ID NO: 1154. In some embodiments, the fusion protein comprising an IL15RA SUSHI domain and an IL-15v comprises an amino acid sequence of SEQ ID NO: 1155. In some embodiments, the fusion protein comprising an IL15RA SUSHI domain and an IL-15v comprises an amino acid sequence of SEQ ID NO: 1156. In some embodiments, the fusion protein comprising an IL15RA SUSHI domain and an IL-15v comprises an amino acid sequence of SEQ ID NO: 1157. In some embodiments, the fusion protein comprising an IL15RA SUSHI domain and an IL-15v comprises an amino acid sequence of SEQ ID NO: 1158. In some embodiments, the fusion protein comprising an IL15RA SUSHI domain and an IL-15v comprises an amino acid sequence of SEQ ID NO: 1159. In some embodiments, the fusion protein comprising an IL15RA SUSHI domain and an IL-15v comprises an amino acid sequence of SEQ ID NO: 1160. In some embodiments, the fusion protein comprising an IL15RA SUSHI domain and an IL-15v comprises an amino acid sequence of SEQ ID NO: 1161. In some embodiments, the fusion protein comprising an IL15RA SUSHI domain and an IL-15v comprises an amino acid sequence of SEQ ID NO: 1162. In some embodiments, the fusion protein comprising an IL15RA SUSHI domain and an IL-15v comprises an amino acid sequence of SEQ ID NO: 1163. In some embodiments, the fusion protein comprising an IL15RA SUSHI domain and an IL-15v comprises an amino acid sequence of SEQ ID NO: 1164. In some embodiments, the fusion protein comprising an IL15RA SUSHI domain and an IL-15v comprises an amino acid sequence of SEQ ID NO: 1165. In some embodiments, the fusion protein comprising an IL15RA SUSHI domain and an IL-15v comprises an amino acid sequence of SEQ ID NO: 1166. In some embodiments, the fusion protein comprising an IL15RA SUSHI domain and an IL-15v comprises an amino acid sequence of SEQ ID NO: 1167. In some embodiments, the fusion protein comprising an IL15RA SUSHI domain and an IL-15v comprises an amino acid sequence of SEQ ID NO: 1168. In some embodiments, the fusion protein comprising an IL15RA SUSHI domain and an IL-15v comprises an amino acid sequence of SEQ ID NO: 1169. In some embodiments, the fusion protein comprising an IL15RA SUSHI domain and an IL-15v comprises an amino acid sequence of SEQ ID NO: 1170. In some embodiments, the fusion protein comprising an IL15RA SUSHI domain and an IL-15v comprises an amino acid sequence of SEQ ID NO: 1171. In some embodiments, the fusion protein comprising an IL15RA SUSHI domain and an IL-15v comprises an amino acid sequence of SEQ ID NO: 1172. In some embodiments, the fusion protein comprising an IL15RA SUSHI domain and an IL-15v comprises an amino acid sequence of SEQ ID NO: 1173. In some embodiments, the fusion protein comprising an IL15RA SUSHI domain and an IL-15v comprises an amino acid sequence of SEQ ID NO: 1174. In some embodiments, the fusion protein comprising an IL15RA SUSHI domain and an IL-15v comprises an amino acid sequence of SEQ ID NO: 1175. In some embodiments, the fusion protein comprising an IL15RA SUSHI domain and an IL-15v comprises an amino acid sequence of SEQ ID NO: 1176. In some embodiments, the fusion protein comprising an IL15RA SUSHI domain and an IL-15v comprises an amino acid sequence of SEQ ID NO: 1177. In some embodiments, the fusion protein comprising an IL15RA SUSHI domain and an IL-15v comprises an amino acid sequence of SEQ ID NO: 1178. In some embodiments, the fusion protein comprising an IL15RA SUSHI domain and an IL-15v comprises an amino acid sequence of SEQ ID NO: 1179. In some embodiments, the fusion protein comprising an IL15RA SUSHI domain and an IL-15v comprises an amino acid sequence of SEQ ID NO: 1180. In some embodiments, the fusion protein comprising an IL15RA SUSHI domain and an IL-15v comprises an amino acid sequence of SEQ ID NO: 1181. In some embodiments, the fusion protein comprising an IL15RA SUSHI domain and an IL-15v comprises an amino acid sequence of SEQ ID NO: 1182. In some embodiments, the fusion protein comprising an IL15RA SUSHI domain and an IL-15v comprises an amino acid sequence of SEQ ID NO: 1183. In some embodiments, the fusion protein comprising an IL15RA SUSHI domain and an IL-15v comprises an amino acid sequence of SEQ ID NO: 1184. In some embodiments, the fusion protein comprising an IL15RA SUSHI domain and an IL-15v comprises an amino acid sequence of SEQ ID NO: 1185. In some embodiments, the fusion protein comprising an IL15RA SUSHI domain and an IL-15v comprises an amino acid sequence of SEQ ID NO: 1186. In some embodiments, the fusion protein comprising an IL15RA SUSHI domain and an IL-15v comprises an amino acid sequence of SEQ ID NO: 1187. In some embodiments, the fusion protein comprising an IL15RA SUSHI domain and an IL-15v comprises an amino acid sequence of SEQ ID NO: 1188. In some embodiments, the fusion protein comprising an IL15RA SUSHI domain and an IL-15v comprises an amino acid sequence of SEQ ID NO: 1189. In some embodiments, the fusion protein comprising an IL15RA SUSHI domain and an IL-15v comprises an amino acid sequence of SEQ ID NO: 1190. In some embodiments, the fusion protein comprising an IL15RA SUSHI domain and an IL-15v comprises an amino acid sequence of SEQ ID NO: 1191. In some embodiments, the fusion protein comprising an IL15RA SUSHI domain and an IL-15v comprises an amino acid sequence of SEQ ID NO: 1192. In some embodiments, the fusion protein comprising an IL15RA SUSHI domain and an IL-15v comprises an amino acid sequence of SEQ ID NO: 1193. In some embodiments, the fusion protein comprising an IL15RA SUSHI domain and an IL-15v comprises an amino acid sequence of SEQ ID NO: 1194. In some embodiments, the fusion protein comprising an IL15RA SUSHI domain and an IL-15v comprises an amino acid sequence of SEQ ID NO: 1195. In some embodiments, the fusion protein comprising an IL15RA SUSHI domain and an IL-15v comprises an amino acid sequence of SEQ ID NO: 1196. In some embodiments, the fusion protein comprising an IL15RA SUSHI domain and an IL-15v comprises an amino acid sequence of SEQ ID NO: 1197. In some embodiments, the fusion protein comprising an IL15RA SUSHI domain and an IL-15v comprises an amino acid sequence of SEQ ID NO: 1198. In some embodiments, the fusion protein comprising an IL15RA SUSHI domain and an IL-15v comprises an amino acid sequence of SEQ ID NO: 1199. In some embodiments, the fusion protein comprising an IL15RA SUSHI domain and an IL-15v comprises an amino acid sequence of SEQ ID NO: 1200. In some embodiments, the fusion protein comprising an IL15RA SUSHI domain and an IL-15v comprises an amino acid sequence of SEQ ID NO: 1201. In some embodiments, the fusion protein comprising an IL15RA SUSHI domain and an IL-15v comprises an amino acid sequence of SEQ ID NO: 1202. In some embodiments, the fusion protein comprising an IL15RA SUSHI domain and an IL-15v comprises an amino acid sequence of SEQ ID NO: 1203. In some embodiments, the fusion protein comprising an IL15RA SUSHI domain and an IL-15v comprises an amino acid sequence of SEQ ID NO: 1204. In some embodiments, the fusion protein comprising an IL15RA SUSHI domain and an IL-15v comprises an amino acid sequence of SEQ ID NO: 1205. In some embodiments, the fusion protein comprising an IL15RA SUSHI domain and an IL-15v comprises an amino acid sequence of SEQ ID NO: 1206. In some embodiments, the fusion protein comprising an IL15RA SUSHI domain and an IL-15v comprises an amino acid sequence of SEQ ID NO: 1207. In some embodiments, the fusion protein comprising an IL15RA SUSHI domain and an IL-15v comprises an amino acid sequence of SEQ ID NO: 1208.

In some embodiments, the IL15RA SUSHI domain-IL-15v fusion protein binds to IL-2Rβ with a binding equilibrium dissociation constant (K_{D}) of at least 1 μM, e.g., a K_{D} of at least 1.5 μM, at least 2 μM, at least 2.5 μM, at least 3 μM, at least 3.5 μM, at least 4 μM, at least 4.5 μM or at least 5 μM. In some embodiments, the IL15RA SUSHI domain-IL-15v fusion protein binds to IL-2Rβγ with a binding equilibrium dissociation constant (K_{D}) of at least 1 μM, e.g., a K_{D} of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM. In some embodiments, the IL15RA SUSHI domain-IL-15v fusion proteins described herein induce CD4+ T cell proliferation with an EC50 of less than 5 nM, e.g., less than 4 nM, less than 3 nM, less than 2 nM, less than 1 nM, e.g., wherein CD4+ T cell proliferation potency is measured by marker of proliferation Ki-67 activation (MK167; NCBI Gene ID: 4288). In some embodiments, the IL15RA SUSHI domain-IL-15v fusion protein induces CD8+ T cells and/or natural killer (NK) cell proliferation with an EC50 of greater than 100 nM, e.g., wherein CD8+ T cell and/or NK cell proliferation potency is measured by Ki-67 activation. In some embodiments, the IL15RA SUSHI domain-IL-15v fusion protein described herein induce CD4+ T cell proliferation with a potency that is at least 100-fold, e.g., at least 200-fold, at least 300-fold, at least 400-fold, at least 500-fold, at least 600-fold, or more, in comparison to the potency for inducing CD8+ T cell or NK cell proliferation, e.g., wherein cell proliferation potency is measured by Ki-67 activation.

Illustrative fusion protein comprising an IL15RA SUSHI domain and an IL-15v are provided in Table E, below. The IL-15RA SUSHI-IL-15v fusion proteins provided in Table E do not have any non-naturally occurring cysteine residues.

c. Serum Half-Life Extended IL-15v

Various strategies for extending or prolonging the serum half-life of a biologic molecule include PEGylation, peptide moieties (e.g., albumin, XTEN), and amino acid substitutions to the Fc domain. Provided are IL-15v having extended serum half-life, e.g., in comparison to wild-type IL-15. In various embodiments, the IL-15v and fusion proteins comprising an IL15RA SUSHI domain and an IL-15v, described herein, are PEGylated.

In some embodiments, the herein described IL-15v and IL-15RA SUSHI-IL-15v fusion proteins are further linked or fused to a serum half-life extending polypeptide. Polypeptides that can be used to extend the serum half-life of another polypeptide, e.g., via linking or fusion, are known in the art and can be used in the present fusion proteins. Illustrative serum half-life extending polypeptides that can be linked or fused with the herein described IL-15v and IL-15RA SUSHI-IL-15v fusion proteins include without limitation an immunoglobulin fragment crystallizable region (Fc region), one or more serum albumin moieties, an albumin binding protein or peptide, an IgG, an XTEN polypeptide, a proline/alanine/serine-rich (PAS) polypeptide, an elastin-like polypeptide. IL-15v and IL-15RA SUSHI-IL-15v fusion proteins linked or fused to one or more serum albumin moieties, an albumin binding protein or peptide, an IgG, an XTEN polypeptide, a proline/alanine/serine-rich (PAS) polypeptide, an elastin-like polypeptide need not dimerize (e.g., need not form homodimers or heterodimers). The one or more serum albumin moieties, an albumin binding protein or peptide, an IgG, an XTEN polypeptide, a proline/alanine/serine-rich (PAS) polypeptide, an elastin-like polypeptide can be linked or fused to either or both of the N-terminus or the C-terminus of the IL-15v or IL-15RA SUSHI-IL-15v fusion proteins. Illustrative XTEN protein polymers that can be used in the present IL-15v and IL-15RA SUSHI-IL-15v fusion proteins are described, e.g., in Schellenberger, et al., Nat Biotechnol. 2009 December; 27(12):1186-90; Podust, et al., Journal of Controlled Release 240 (2016) 52-66; WO2010091122, WO2011123813, WO2013130683, WO2016077505 and WO2017197048. Illustrative proline/alanine/serine-rich (PAS) polypeptides that can be used in the present IL-2v fusion proteins are described, e.g., in Schlapschy, et al., Protein Eng Des Sel. (2013) 26(8):489-501 and Breibeck, et al., Biopolymers. (2018) January; 109(1). doi: 10.1002/bip.23069, WO2016122806 and WO2016130451. The foregoing references are which are hereby incorporated herein by reference in their entirety for all purposes.

In some embodiments, the serum half-life extending polypeptide is an immunoglobulin fragment crystallizable region (Fc region). Generally, the Fc domain is comprised of or derived from the same species as the IL-15v or IL-15RA SUSHI-IL-15v fusion protein domain (e.g., human, dog, cat, mouse or monkey). In some embodiments, the Fc region is from a human IgG1, IgG2, IgG3 or IgG4. In some embodiments, the Fc region is from a human IgG1 or IgG4.

In some embodiments, the Fc modifications can promote one or more of increased serum half-life or decreased antibody effector function of the molecule. In other embodiments, certain of these modifications, decrease antibody effector function and increase half-life of the antibody. In some embodiments, the Fc fusion proteins with IL-15v or IL-15RA SUSHI-IL-15v fusion proteins described herein comprise two or more, three or more, four or more, five or more, six or more, six or fewer, five or fewer, four or fewer, three or fewer, two or fewer, or one modified Fc amino acid residue(s). Exemplary amino acid substitutions are described below.

To promote extended serum half-life of the IL-15v or IL-15RA SUSHI-IL-15v Fc fusion proteins, in some embodiments, the Fc domain comprises amino acid modifications that promote an increased serum half-life of the anti-binding molecule. Amino acid substitutions that increase the half-life of an antibody have been described. In one embodiment, the Fc region or Fc domain comprises a methionine to tyrosine substitution at position 252 (Eu numbering), a serine to threonine substitution at position 254 (Eu numbering), and a threonine to glutamic acid substitution at position 256 (Eu numbering). See, e.g., U.S. Pat. No. 7,658,921. This type of mutant, designated as a “YTE” exhibits a four-fold increased half-life relative to wild-type versions of the same antibody (Dall'Acqua, et al., J Biol Chem, 281:23514-24 (2006); Robbie, et al., Antimicrob Agents Chemotherap., 57 (12):6147-6153 (2013)). In certain embodiments, the Fc region or Fc domain comprises an IgG constant domain comprising one, two, three or more amino acid substitutions of amino acid residues at positions 251-257, 285-290, 308-314, 385-389, and 428-436 (Eu numbering). Alternatively, M428L and N434S (“LS”) amino acid substitutions can increase the pharmacokinetic half-life of the CD-4 binding IL-15v molecule. In other embodiments, the Fc region or Fc domain of comprises a M428L and N434S substitution (Eu numbering). In other embodiments, the Fc region or Fc domain comprises T250Q and M428L (Eu numbering) amino acid substitutions, e.g., as described in U.S. Pat. Nos. 7,217,797 and 7,217,798. In other embodiments, the Fc region or Fc domain comprises H433K and N434F (Eu numbering) amino acid substitutions, e.g., as described in U.S. Pat. No. 8,163,881. In other embodiments, the Fc region or Fc domain comprises T307Q/Q311V/A378V (DF215) or T256D/N286D/T307R/Q311V/A378V (DF228) (Eu numbering) amino acid substitutions, e.g., as described in U.S. Patent Publ. No. 2020-0277358. In some embodiments, the Fc region or Fc domain comprises aspartic acid at position 309, histidine at position 311 and serine at position 434 (DHS), e.g., as described in U.S. Pat. No. 11,059,892. In some embodiments, the Fc domain comprises serum half-life extending amino acid substitutions at Fc positions (Eu numbering) selected from: tyrosine at position 252, threonine at position 254 and glutamic acid at position 256 (252Y, 254T and 256E); leucine at position 428 (428L); glutamine at position 250 and leucine at position 428 (250Q and 428L); leucine at position 428 and serine at position 434 (428L and 434S); leucine at position 428 and alanine at position 434 (428L and 434A); arginine at position 311 and leucine at position 428 (311R and 428L); glycine at position 309 and leucine at position 428 (309G and 428L); glutamine at position 307, valine at position 311 and valine at position 378 (307Q, 311V and 378V); aspartic acid at position 256, aspartic acid at position 286, arginine at position 307, valine at position 311 and valine at position 378 (256D, 286D, 307R, 311V and 378V). See, e.g., U.S. Pat. Nos. 6,821,505; 6,277,375; 7,083,784; 8,475,792; 7,217,797; 7,217,798; 7,670,600; 8,088,376; 8,394,925; 8,546,543; 9,803,023; 10,336,818; 10,683,368; 11,440,971; 11,059,892; 8,163,881; 8,834,871; 11,492,415; and 11,319,383; and U.S. Patent Publication Nos. 2019/0292269; 2020/0277358; and 2020/0277358, which are hereby incorporated herein by reference in their entireties for all purposes.

In some embodiments, the Fc domain of the fusion protein does not comprise a hinge region; it is truncated or deleted, in whole or in part. The structural hinge region of human IgG1, IgG2 and IgG4 antibodies is a peptide linker of 19 to 23 amino acids containing two to four cysteine residues, is genetically encoded on the hinge exon together with the 5′-end of the CH2 exon, and allows for disulfide bridges between first and second Fc domains (Roux, et al., J. Immunol. (1998) 161:4083). The structural hinge region is comprised of amino acid residue positions 216-238 (Eu numbering) or 226-251 (Kabat numbering) (identified on imgt.org). In some embodiments, the Fc region comprises or is derived from a human lgG4 isotype and does not comprise the amino acid sequence ESKYGPPCPPCP (SEQ ID NO: 1121). In some embodiments, the Fc region comprises or is derived from a human IgG1 isotype and does not comprise the amino acid sequence EPKSCDKTHTCPPCP (SEQ ID NO: 1122) or EPKSCDKTHTCPPCPAPELL (SEQ ID NO: 1123).

d. IL-15v Transmembrane Domain Fusion Protein

Provided is a fusion protein comprising a IL-15v or IL-15RA SUSHI-IL-15v fusion protein, as described herein, fused to a transmembrane domain, e.g., to allow for cell surface expression or presentation of the IL-15v or IL-15RA SUSHI-IL-15v fusion protein. Further provided is a host cell comprising a polynucleotide encoding a IL-15v or IL-15RA SUSHI-IL-15v fusion protein, as described herein, fused to a transmembrane domain.

e. IL-15v Chimeric Antigen Receptors (CARs)

Provided is a chimeric antigen receptors (CAR) comprising a IL-15v or IL-15RA SUSHI-IL-15v fusion protein, as described herein. Illustrative IL-15 CAR fusion proteins are described, e.g., in Zannikou, et al., J Immunother Cancer (2023) 11:e006239. Generally, an IL-15v CAR comprises from the N-terminus to the C-terminus, an extracellular domain comprising an IL-15v or IL-15RA SUSHI-IL-15v fusion protein, as described herein, fused directly or indirectly to a transmembrane domain, fused directly or indirectly to an intracellular signaling domain. In one embodiment, the IL-15 CAR fusion protein comprises an IL-15v or IL-15RA SUSHI-IL-15v fusion protein, as described herein, fused directly or indirectly to a CD28 transmembrane domain, fused to a CD28 intracellular signalling domain, fused to a CD3 zeta intracellular signalling domain.

TABLE C
Illustrative IL15RA Sushi domain amino acid sequences

| SEQ ID | IL15RA source | | residues | |
|---|---|---|---|---|
| NO: | NCBIRef Seq: | isoform | | Sushi domain amino acid sequence |
| 734 | | | 59 | MSVEHADIWVKSYSLYSRERYICNSGFKRKAGT |
| | | | | SSLTECVLNKATNVAHWTTPSLKCIX |
| | | | | X = K or R |
| 735 | | | 65 | ITCPPPMSVEHADIWVKSYSLYSRERYICNSGF |
| | | | | KRKAGTSSLTECVLNKATNVAHWTTPSLKCIX |
| | | | | X = K or R |
| 736 | NP_001230468.1 | 3 | 1-59 | MSVEHADIWVKSYSLYSRERYICNSGFKRKAGT |
| | NP_001338025.1 | 6 | 1-59 | SSLTECVLNKATNVAHWTTPSLKCIR |
| 737 | NP_001338026.1 | 7 | 1-59 | MSVEHADIWVKSYSLYSRERYICNSGFKRKAGT |
| | | | | SSLTECVLNKATNVAHWTTPSLKCIK |
| 738 | NP_002180.1 | 1 precursor | 31-95 | ITCPPPMSVEHADIWVKSYSLYSRERYICNSGF |
| | NP_001243694 | 4 | 117-181 | KRKAGTSSLTECVLNKATNVAHWTTPSLKCIR |
| 739 | NP 751950.2 | 2 precursor | 31-95 | ITCPPPMSVEHADIWVKSYSLYSRERYICNSGF |
| | NP_001338024.1 | 5 | 89-153 | KRKAGTSSLTECVLNKATNVAHWTTPSLKCIK |

TABLE D
Illustrative IL-15 variants (IL-15v)

| SEQ ID | | |
|---|---|---|
| NO: | Features | sequence |
| 740 | Wild- | NWVNVISDLKKIEDLIQSMHIDATLYTESDVHPSCKVTAMKCFLLELQVISLESGDASIHDTVE |
| | type | NLIILANNSLSSNGNVTESGCKECEELEEKNIKEFLQSFVHIVQMFINTS |
| 741 | N1D | DWVNVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 742 | N4D | NWVDVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 743 | S7G | NWVNVIGDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 744 | D8G | NWVNVISGLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 745 | D8N | NWVNVISNLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 746 | D30N | NWVNVISDLKKIEDLIQSMHIDATLYTESNVHPSCKVTAMKCFLLELQVISLESGDASIHDTVE |
| | | NLIILANNSLSSNGNVTESGCKECEELEEKNIKEFLQSFVHIVQMFINTS |
| 747 | D61N | NWVNVISDLKKIEDLIQSMHIDATLYTESDVHPSCKVTAMKCFLLELQVISLESGDASIHNTVE |
| | | NLIILANNSLSSNGNVTESGCKECEELEEKNIKEFLQSFVHIVQMFINTS |
| 748 | E64Q | NWVNVISDLKKIEDLIQSMHIDATLYTESDVHPSCKVTAMKCFLLELQVISLESGDASIHDTVQ |
| | | NLIILANNSLSSNGNVTESGCKECEELEEKNIKEFLQSFVHIVQMFINTS |
| 749 | N65D | NWVNVISDLKKIEDLIQSMHIDATLYTESDVHPSCKVTAMKCELLELQVISLESGDASIHDTVE |
| | | DLIILANNSLSSNGNVTESGCKECEELEEKNIKEFLQSFVHIVQMFINTS |
| 750 | I68A | NWVNVISDLKKIEDLIQSMHIDATLYTESDVHPSCKVTAMKCFLLELQVISLESGDASIHDTVE |
| | | NLIALANNSLSSNGNVTESGCKECEELEEKNIKEFLQSFVHIVQMFINTS |
| 751 | N71L | NWVNVISDLKKIEDLIQSMHIDATLYTESDVHPSCKVTAMKCELLELQVISLESGDASIHDTVE |
| | | NLIILALNSLSSNGNVTESGCKECEELEEKNIKEFLQSFVHIVQMFINTS |
| 752 | N71E | NWVNVISDLKKIEDLIQSMHIDATLYTESDVHPSCKVTAMKCFLLELQVISLESGDASIHDTVE |
| | | NLIILAENSLSSNGNVTESGCKECEELEEKNIKEFLQSFVHIVQMFINTS |
| 753 | N72D | NWVNVISDLKKIEDLIQSMHIDATLYTESDVHPSCKVTAMKCELLELQVISLESGDASIHDTVE |
| | | NLIILANDSLSSNGNVTESGCKECEELEEKNIKEFLQSFVHIVQMFINTS |
| 754 | N77L | NWVNVISDLKKIEDLIQSMHIDATLYTESDVHPSCKVTAMKCFLLELQVISLESGDASIHDTVE |
| | | NLIILANNSLSSLGNVTESGCKECEELEEKNIKEFLQSFVHIVQMFINTS |
| 755 | N79G | NWVNVISDLKKIEDLIQSMHIDATLYTESDVHPSCKVTAMKCFLLELQVISLESGDASIHDTVE |
| | | NLIILANNSLSSNGGVTESGCKECEELEEKNIKEFLQSFVHIVQMFINTS |
| 756 | N79P | NWVNVISDLKKIEDLIQSMHIDATLYTESDVHPSCKVTAMKCFLLELQVISLESGDASIHDTVE |
| | | NLIILANNSLSSNGPVTESGCKECEELEEKNIKEFLQSFVHIVQMFINTS |
| 757 | Q108E | NWVNVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVEMFINTS |
| 758 | N112K | NWVNVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFIKTS |
| 759 | N112D | NWVNVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFIDTS |
| 760 | N112S | NWVNVISDLKKIEDLIQSMHIDATLYTESDVHPSCKVTAMKCFLLELQVISLESGDASIHDTVE |
| | | NLIILANNSLSSNGNVTESGCKECEELEEKNIKEFLQSFVHIVQMFISTS |
| 761 | N1D/N4D/ | DWVDVISNLKKIEDLIQSMHIDATLYTESDVHPSCKVTAMKCFLLELQVISLESGDASIHDTVE |
| | D8N | NLIILANNSLSSNGNVTESGCKECEELEEKNIKEFLQSFVHIVQMFINTS |
| 762 | N1D/D61N | DWVNVISDLKKIEDLIQSMHIDATLYTESDVHPSCKVTAMKCFLLELQVISLESGDASIHNTVE |
| | | NLIILANNSLSSNGNVTESGCKECEELEEKNIKEFLQSFVHIVQMFINTS |
| 763 | N1D/E64Q | DWVNVISDLKKIEDLIQSMHIDATLYTESDVHPSCKVTAMKCELLELQVISLESGDASIHDTVQ |
| | | NLIILANNSLSSNGNVTESGCKECEELEEKNIKEFLQSFVHIVQMFINTS |
| 764 | N1D/N65D | DWVNVISDLKKIEDLIQSMHIDATLYTESDVHPSCKVTAMKCFLLELQVISLESGDASIHDTVE |
| | | DLIILANNSLSSNGNVTESGCKECEELEEKNIKEFLQSFVHIVQMFINTS |
| 1124 | N-term | DNWVNVISDLKKIEDLIQSMHIDATLYTESDVHPSCKVTAMKCFLLELQVISLESGDASIHDTV |
| | D/N72D | ENLIILANDSLSSNGNVTESGCKECEELEEKNIKEFLQSFVHIVQMFINTS |
| 765 | N4D/D61N | NWVDVISDLKKIEDLIQSMHIDATLYTESDVHPSCKVTAMKCFLLELQVISLESGDASIHNTVE |
| | | NLIILANNSLSSNGNVTESGCKECEELEEKNIKEFLQSFVHIVQMFINTS |
| 766 | N4D/E64Q | NWVDVISDLKKIEDLIQSMHIDATLYTESDVHPSCKVTAMKCFLLELQVISLESGDASIHDTVQ |
| | | NLIILANNSLSSNGNVTESGCKECEELEEKNIKEFLQSFVHIVQMFINTS |
| 767 | N4D/N65D | NWVDVISDLKKIEDLIQSMHIDATLYTESDVHPSCKVTAMKCFLLELQVISLESGDASIHDTVE |
| | | DLIILANNSLSSNGNVTESGCKECEELEEKNIKEFLQSFVHIVQMFINTS |
| 768 | S7G/N65D | NWVNVIGDLKKIEDLIQSMHIDATLYTESDVHPSCKVTAMKCFLLELQVISLESGDASIHDTVE |
| | | DLIILANNSLSSNGNVTESGCKECEELEEKNIKEFLQSFVHIVQMFINTS |
| 769 | D8G/D61N | NWVNVISGLKKIEDLIQSMHIDATLYTESDVHPSCKVTAMKCFLLELQVISLESGDASIHNTVE |
| | | NLIILANNSLSSNGNVTESGCKECEELEEKNIKEFLQSFVHIVQMFINTS |
| 770 | D8G/E64Q | NWVNVISGLKKIEDLIQSMHIDATLYTESDVHPSCKVTAMKCFLLELQVISLESGDASIHDTVQ |
| | | NLIILANNSLSSNGNVTESGCKECEELEEKNIKEFLQSFVHIVQMFINTS |
| 771 | D8G/N65D | NWVNVISGLKKIEDLIQSMHIDATLYTESDVHPSCKVTAMKCFLLELQVISLESGDASIHDTVE |
| | | DLIILANNSLSSNGNVTESGCKECEELEEKNIKEFLQSFVHIVQMFINTS |
| 772 | D8N/D61N | NWVNVISNLKKIEDLIQSMHIDATLYTESDVHPSCKVTAMKCFLLELQVISLESGDASIHNTVE |
| | | NLIILANNSLSSNGNVTESGCKECEELEEKNIKEFLQSFVHIVQMFINTS |
| 773 | D8N/E64Q | NWVNVISNLKKIEDLIQSMHIDATLYTESDVHPSCKVTAMKCFLLELQVISLESGDASIHDTVQ |
| | | NLIILANNSLSSNGNVTESGCKECEELEEKNIKEFLQSFVHIVQMFINTS |
| 774 | D8N/N65D | NWVNVISNLKKIEDLIQSMHIDATLYTESDVHPSCKVTAMKCFLLELQVISLESGDASIHDTVE |
| | | DLIILANNSLSSNGNVTESGCKECEELEEKNIKEFLQSFVHIVQMFINTS |
| 775 | D30N/N65D | NWVNVISDLKKIEDLIQSMHIDATLYTESNVHPSCKVTAMKCFLLELQVISLESGDASIHDTVE |
| | | DLIILANNSLSSNGNVTESGCKECEELEEKNIKEFLQSFVHIVQMFINTS |
| 776 | D61N/E64Q | NWVNVISDLKKIEDLIQSMHIDATLYTESDVHPSCKVTAMKCELLELQVISLESGDASIHNTVQ |
| | | NLIILANNSLSSNGNVTESGCKECEELEEKNIKEFLQSFVHIVQMFINTS |
| 777 | D61N/N65D | NWVNVISDLKKIEDLIQSMHIDATLYTESDVHPSCKVTAMKCFLLELQVISLESGDASIHNTVE |
| | | DLIILANNSLSSNGNVTESGCKECEELEEKNIKEFLQSFVHIVQMFINTS |
| 778 | E64Q/N65D | NWVNVISDLKKIEDLIQSMHIDATLYTESDVHPSCKVTAMKCELLELQVISLESGDASIHDTVQ |
| | | DLIILANNSLSSNGNVTESGCKECEELEEKNIKEFLQSFVHIVQMFINTS |
| 1125 | N65A/I68A | NWVNVISDLKKIEDLIQSMHIDATLYTESDVHPSCKVTAMKCELLELQVISLESGDASIHDTVE |
| | | ALIALANNSLSSNGNVTESGCKECEELEEKNIKEFLQSFVHIVQMFINTS |
| 779 | N65D/I68A | NWVNVISDLKKIEDLIQSMHIDATLYTESDVHPSCKVTAMKCFLLELQVISLESGDASIHDTVE |
| | | DLIALANNSLSSNGNVTESGCKECEELEEKNIKEFLQSFVHIVQMFINTS |
| 780 | E64Q/Q108E | NWVNVISDLKKIEDLIQSMHIDATLYTESDVHPSCKVTAMKCFLLELQVISLESGDASIHDTVQ |
| | | NLIILANNSLSSNGNVTESGCKECEELEEKNIKEFLQSFVHIVEMFINTS |
| 781 | D30N/E64Q/ | NWVNVISDLKKIEDLIQSMHIDATLYTESNVHPSCKVTAMKCFLLELQVISLESGDASIHDTVQ |
| | N65D | DLIILANNSLSSNGNVTESGCKECEELEEKNIKEFLQSFVHIVQMFINTS |
| 782 | D61N/E64Q/ | NWVNVISDLKKIEDLIQSMHIDATLYTESDVHPSCKVTAMKCFLLELQVISLESGDASIHNTVQ |
| | N65D | DLIILANNSLSSNGNVTESGCKECEELEEKNIKEFLQSFVHIVQMFINTS |
| 783 | D61N/N65D/ | NWVNVISDLKKIEDLIQSMHIDATLYTESDVHPSCKVTAMKCFLLELQVISLESGDASIHNTVE |
| | I68A | DLIALANNSLSSNGNVTESGCKECEELEEKNIKEFLQSFVHIVQMFINTS |
| 784 | N71L/N79P | NWVNVISDLKKIEDLIQSMHIDATLYTESDVHPSCKVTAMKCFLLELQVISLESGDASIHDTVE |
| | | NLIILALNSLSSNGPVTESGCKECEELEEKNIKEFLQSFVHIVQMFINTS |
| 785 | N71L/N112D | NWVNVISDLKKIEDLIQSMHIDATLYTESDVHPSCKVTAMKCFLLELQVISLESGDASIHDTVE |
| | | NLIILALNSLSSNGNVTESGCKECEELEEKNIKEFLQSFVHIVQMFIDTS |
| 786 | N79P/N112D | NWVNVISDLKKIEDLIQSMHIDATLYTESDVHPSCKVTAMKCFLLELQVISLESGDASIHDTVE |
| | | NLIILANNSLSSNGPVTESGCKECEELEEKNIKEFLQSFVHIVQMFIDTS |
| 787 | S7G/N65A/ | NWVNVIGDLKKIEDLIQSMHIDATLYTESDVHPSCKVTAMKCFLLELQVISLESGDASIHDTVE |
| | I68A | ALIALANNSLSSNGNVTESGCKECEELEEKNIKEFLQSFVHIVQMFINTS |
| 788 | S7G/N65D/ | NWVNVIGDLKKIEDLIQSMHIDATLYTESDVHPSCKVTAMKCFLLELQVISLESGDASIHDTVE |
| | I68A | DLIALANNSLSSNGNVTESGCKECEELEEKNIKEFLQSFVHIVQMFINTS |
| 789 | N71Q/N79Q/ | NWVNVISDLKKIEDLIQSMHIDATLYTESDVHPSCKVTAMKCFLLELQVISLESGDASIHDTVE |
| | N112Q | NLIILAQNSLSSNGQVTESGCKECEELEEKNIKEFLQSFVHIVQMFIQTS |
| 790 | N71A/N79A/ | NWVNVISDLKKIEDLIQSMHIDATLYTESDVHPSCKVTAMKCFLLELQVISLESGDASIHDTVE |
| | N112A | NLIILAANSLSSNGAVTESGCKECEELEEKNIKEFLQSFVHIVQMFIATS |
| 791 | N71G/N79G/ | NWVNVISDLKKIEDLIQSMHIDATLYTESDVHPSCKVTAMKCFLLELQVISLESGDASIHDTVE |
| | N112G | NLIILAGNSLSSNGGVTESGCKECEELEEKNIKEFLQSFVHIVQMFIGTS |
| 792 | N71S/N79L/ | NWVNVISDLKKIEDLIQSMHIDATLYTESDVHPSCKVTAMKCFLLELQVISLESGDASIHDTVE |
| | N112E | NLIILASNSLSSNGLVTESGCKECEELEEKNIKEFLQSFVHIVQMFIETS |
| 793 | N71E/N79P/ | NWVNVISDLKKIEDLIQSMHIDATLYTESDVHPSCKVTAMKCELLELQVISLESGDASIHDTVE |
| | N112K | NLIILAENSLSSNGPVTESGCKECEELEEKNIKEFLQSFVHIVQMFIKTS |
| 794 | N71L/N79P/ | NWVNVISDLKKIEDLIQSMHIDATLYTESDVHPSCKVTAMKCFLLELQVISLESGDASIHDTVE |
| | N112K | NLIILALNSLSSNGPVTESGCKECEELEEKNIKEFLQSFVHIVQMFIKTS |
| 795 | N71L/N79P/ | NWVNVISDLKKIEDLIQSMHIDATLYTESDVHPSCKVTAMKCELLELQVISLESGDASIHDTVE |
| | N112D | NLIILALNSLSSNGPVTESGCKECEELEEKNIKEFLQSFVHIVQMFIDTS |
| 796 | N71K/N79P/ | NWVNVISDLKKIEDLIQSMHIDATLYTESDVHPSCKVTAMKCFLLELQVISLESGDASIHDTVE |
| | N112D | NLIILAKNSLSSNGPVTESGCKECEELEEKNIKEFLQSFVHIVQMFIDTS |
| 797 | N71E/N79P/ | NWVNVISDLKKIEDLIQSMHIDATLYTESDVHPSCKVTAMKCFLLELQVISLESGDASIHDTVE |
| | N112S | NLIILAENSLSSNGPVTESGCKECEELEEKNIKEFLQSFVHIVQMFISTS |
| 798 | N71E/N79P/ | NWVNVISDLKKIEDLIQSMHIDATLYTESDVHPSCKVTAMKCFLLELQVISLESGDASIHDTVE |
| | N112D | NLIILAENSLSSNGPVTESGCKECEELEEKNIKEFLQSFVHIVQMFIDTS |
| 799 | N1D/D61N/ | DWVNVISDLKKIEDLIQSMHIDATLYTESDVHPSCKVTAMKCFLLELQVISLESGDASIHNTVQ |
| | E64Q/Q108E | NLIILANNSLSSNGNVTESGCKECEELEEKNIKEFLQSFVHIVEMFINTS |
| 800 | N4D/D61N/ | NWVDVISDLKKIEDLIQSMHIDATLYTESDVHPSCKVTAMKCFLLELQVISLESGDASIHNTVQ |
| | E64Q/Q108E | NLIILANNSLSSNGNVTESGCKECEELEEKNIKEFLQSFVHIVEMFINTS |
| 801 | N71Q/N77L/ | NWVNVISDLKKIEDLIQSMHIDATLYTESDVHPSCKVTAMKCFLLELQVISLESGDASIHDTVE |
| | N79Q/N112Q | NLIILAQNSLSSLGQVTESGCKECEELEEKNIKEFLQSFVHIVQMFIQTS |
| 802 | N71A/N77L/ | NWVNVISDLKKIEDLIQSMHIDATLYTESDVHPSCKVTAMKCFLLELQVISLESGDASIHDTVE |
| | N79A/N112A | NLIILAANSLSSLGAVTESGCKECEELEEKNIKEFLQSFVHIVQMFIATS |
| 803 | N71G/N77L/ | NWVNVISDLKKIEDLIQSMHIDATLYTESDVHPSCKVTAMKCFLLELQVISLESGDASIHDTVE |
| | N79G/N112G | NLIILAGNSLSSLGGVTESGCKECEELEEKNIKEFLQSFVHIVQMFIGTS |
| 804 | N71S/N77L/ | NWVNVISDLKKIEDLIQSMHIDATLYTESDVHPSCKVTAMKCFLLELQVISLESGDASIHDTVE |
| | N79L/N112E | NLIILASNSLSSLGLVTESGCKECEELEEKNIKEFLQSFVHIVQMFIETS |
| 805 | N71E/N77L/ | NWVNVISDLKKIEDLIQSMHIDATLYTESDVHPSCKVTAMKCFLLELQVISLESGDASIHDTVE |
| | N79P/N112K | NLIILAENSLSSLGPVTESGCKECEELEEKNIKEFLQSFVHIVQMFIKTS |
| 806 | N71L/N77L/ | NWVNVISDLKKIEDLIQSMHIDATLYTESDVHPSCKVTAMKCELLELQVISLESGDASIHDTVE |
| | N79P/N112K | NLIILALNSLSSLGPVTESGCKECEELEEKNIKEFLQSFVHIVQMFIKTS |
| 807 | N71E/N77L/ | NWVNVISDLKKIEDLIQSMHIDATLYTESDVHPSCKVTAMKCFLLELQVISLESGDASIHDTVE |
| | N79P/N112S | NLIILAENSLSSLGPVTESGCKECEELEEKNIKEFLQSFVHIVQMFISTS |
| 808 | N71E/N77L/ | NWVNVISDLKKIEDLIQSMHIDATLYTESDVHPSCKVTAMKCFLLELQVISLESGDASIHDTVE |
| | N79P/N112D | NLIILAENSLSSLGPVTESGCKECEELEEKNIKEFLQSFVHIVQMFIDTS |
| 809 | S7G/N65D/ | NWVNVIGDLKKIEDLIQSMHIDATLYTESDVHPSCKVTAMKCFLLELQVISLESGDASIHDTVE |
| | N71L/N79P/ | DLIILALNSLSSNGPVTESGCKECEELEEKNIKEFLQSFVHIVQMFIDTS |
| | N112D | |
| 810 | S7G/N65D/ | NWVNVIGDLKKIEDLIQSMHIDATLYTESDVHPSCKVTAMKCFLLELQVISLESGDASIHDTVE |
| | N71E/N77L/ | DLIILAENSLSSLGPVTESGCKECEELEEKNIKEFLQSFVHIVQMFIDTS |
| | N79P/N112D | |
| 811 | S7G/N65D/ | NWVNVIGDLKKIEDLIQSMHIDATLYTESDVHPSCKVTAMKCFLLELQVISLESGDASIHDTVE |
| | N71E/N77L/ | DLIILAENSLSSLGPVTESGCKECEELEEKNIKEFLQSFVHIVQMFISTS |
| | N79P/N112S | |
| 812 | S7G/N65D/ | NWVNVIGDLKKIEDLIQSMHIDATLYTESDVHPSCKVTAMKCFLLELQVISLESGDASIHDTVE |
| | N71L/N77L/ | DLIILALNSLSSLGPVTESGCKECEELEEKNIKEFLQSFVHIVQMFIKTS |
| | N79P/N112K | |
| 813 | S7G/N65D/ | NWVNVIGDLKKIEDLIQSMHIDATLYTESDVHPSCKVTAMKCFLLELQVISLESGDASIHDTVE |
| | N71E/N77L/ | DLIILAENSLSSLGPVTESGCKECEELEEKNIKEFLQSFVHIVQMFIKTS |
| | N79P/N112K | |

TABLE E
Illustrative IL-15RA Sushi domain-IL-15v fusion proteins

| Features | sequence |
|---|---|
| Wild-type | SEQ ID NO: 814 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1133 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| N1D | SEQ ID NO: 815 |
| | MSVEHADIWVKSYSLYSRERYICNSGFKRKAGTSSLTECVLNKATNVAHWTTPSLKCIX₁(GGX₂S)₁₋₅DWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1134 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| N4D | SEQ ID NO: 816 |
| | MSVEHADIWVKSYSLYSRERYICNSGFKRKAGTSSLTECVLNKATNVAHWTTPSLKCIX₁(GGX₂S)₁₋₅NWVDVIS |
| | DLKKIEDLIQSMHIDATLYTESDVHPSCKVTAMKCELLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1135 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVDVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| S7G | SEQ ID NO: 817 |
| | MSVEHADIWVKSYSLYSRERYICNSGFKRKAGTSSLTECVLNKATNVAHWTTPSLKCIX₁(GGX₂S)₁₋₅NWVNVIG |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1136 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| D8G | SEQ ID NO: 818 |
| | MSVEHADIWVKSYSLYSRERYICNSGFKRKAGTSSLTECVLNKATNVAHWTTPSLKCIX₁(GGX₂S)₁₋₅NWVNVIS |
| | GLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1137 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISGLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| D8N | SEQ ID NO: 819 |
| | MSVEHADIWVKSYSLYSRERYICNSGFKRKAGTSSLTECVLNKATNVAHWTTPSLKCIX₁(GGX₂S)₁₋₅NWVNVIS |
| | NLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1138 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISNLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| D30N | SEQ ID NO: 820 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESNVHPSCKVTAMKCFLLELQVISLESGDASIHDTVENLIILANNSLSSNGNVTE |
| | SGCKECEELEEKNIKEFLQSFVHIVQMFINTS |
| | X₁ = K or R; X₂ = G or GG |
| | SEQ ID NO: 1139 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESNVHPSCKVTAMKCFLLELQVISLESGDASIHDTVENL |
| | IILANNSLSSNGNVTESGCKECEELEEKNIKEFLQSFVHIVQMFINTS |
| D61N | SEQ ID NO: 821 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHNTVENLIILANNSLSSNGNVTE |
| | SGCKECEELEEKNIKEFLQSFVHIVQMFINTS |
| | X₁ = K or R; X₂ = G or GG |
| | SEQ ID NO: 1140 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHNTVENL |
| | IILANNSLSSNGNVTESGCKECEELEEKNIKEFLQSFVHIVQMFINTS |
| E64Q | SEQ ID NO: 822 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVQMFINTS |
| | X₁ = K or R; X₂ = G or GG |
| | SEQ ID NO: 1141 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVQMFINTS |
| N65D | SEQ ID NO: 823 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1142 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVEDL |
| | IILANNSLSSNGNVTESGCKECEELEEKNIKEFLQSFVHIVQMFINTS |
| I68A | SEQ ID NO: 824 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIALANNSLSSNGNVTE |
| | SGCKECEELEEKNIKEFLQSFVHIVQMFINTS |
| | X₁ = K or R; X₂ = G or GG |
| | SEQ ID NO: 1143 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IALANNSLSSNGNVTESGCKECEELEEKNIKEFLQSFVHIVQMFINTS |
| N71L | SEQ ID NO: 825 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSNGNVTE |
| | SGCKECEELEEKNIKEFLQSFVHIVQMFINTS |
| | X₁ = K or R; X₂ = G or GG |
| | SEQ ID NO: 1144 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILALNSLSSNGNVTESGCKECEELEEKNIKEFLQSFVHIVQMFINTS |
| N71E | SEQ ID NO: 826 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSNGNVTE |
| | SGCKECEELEEKNIKEFLQSFVHIVQMFINTS |
| | X₁ = K or R; X₂ = G or GG |
| | SEQ ID NO: 1145 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSNGNVTESGCKECEELEEKNIKEFLQSFVHIVQMFINTS |
| N72D | SEQ ID NO: 827 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANDSLSSNGNVTE |
| | SGCKECEELEEKNIKEFLQSFVHIVQMFINTS |
| | X₁ = K or R; X₂ = G or GG |
| | SEQ ID NO: 1146 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANDSLSSNGNVTESGCKECEELEEKNIKEFLQSFVHIVQMFINTS |
| N77L | SEQ ID NO: 828 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSLGNVTE |
| | SGCKECEELEEKNIKEFLQSFVHIVQMFINTS |
| | X₁ = K or R; X₂ = G or GG |
| | SEQ ID NO: 1147 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSLGNVTESGCKECEELEEKNIKEFLQSFVHIVQMFINTS |
| N79G | SEQ ID NO: 829 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGGVTE |
| | SGCKECEELEEKNIKEFLQSFVHIVQMFINTS |
| | X₁ = K or R; X₂ = G or GG |
| | SEQ ID NO: 1148 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGGVTESGCKECEELEEKNIKEFLQSFVHIVQMFINTS |
| N79P | SEQ ID NO: 830 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGPVTE |
| | SGCKECEELEEKNIKEFLQSFVHIVQMFINTS |
| | X₁ = K or R; X₂ = G or GG |
| | SEQ ID NO: 1149 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGPVTESGCKECEELEEKNIKEFLQSFVHIVQMFINTS |
| Q108E | SEQ ID NO: 831 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVEMFINTS |
| | X₁ = K or R; X₂ = G or GG |
| | SEQ ID NO: 1150 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVEMFINTS |
| N112K | SEQ ID NO: 832 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFIKTS |
| | X₁ = K or R; X₂ = G or GG |
| | SEQ ID NO: 1151 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFIKTS |
| N112D | SEQ ID NO: 833 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFIDTS |
| | X₁ = K or R; X₂ = G or GG |
| | SEQ ID NO: 1152 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFIDTS |
| N112S | SEQ ID NO: 834 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFISTS |
| | X₁ = K or R; X₂ = G or GG |
| | SEQ ID NO: 1153 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFISTS |
| N1D/N4D/ | SEQ ID NO: 835 |
| D8N | MSVEHADIWVKSYSLYSRERYICNSGFKRKAGTSSLTECVLNKATNVAHWTTPSLKCIX₁(GGX₂S)₁₋₅DWVDVIS |
| | NLKKIEDLIQSMHIDATLYTESDVHPSCKVTAMKCFLLELQVISLESGDASIHDTVENLIILANNSLSSNGNVTE |
| | SGCKECEELEEKNIKEFLQSFVHIVQMFINTS |
| | X1 = K or R; X2 = G or GG |
| | SEQ ID NO: 1154 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVDVISNLKKIEDLIQSMHIDATLYTESDVHPSCKVTAMKCFLLELQVISLESGDASIHDTVENL |
| | IILANNSLSSNGNVTESGCKECEELEEKNIKEFLQSFVHIVQMFINTS |
| N1D/D61N | SEQ ID NO: 836 |
| | MSVEHADIWVKSYSLYSRERYICNSGFKRKAGTSSLTECVLNKATNVAHWTTPSLKCIX₁(GGX₂S)₁₋₅DWVNVIS |
| | DLKKIEDLIQSMHIDATLYTESDVHPSCKVTAMKCFLLELQVISLESGDASIHNTVENLIILANNSLSSNGNVTE |
| | SGCKECEELEEKNIKEFLQSFVHIVQMFINTS |
| | X₁ = K or R; X₂ = G or GG |
| | SEQ ID NO: 1155 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVNVISDLKKIEDLIQSMHIDATLYTESDVHPSCKVTAMKCFLLELQVISLESGDASIHNTVENL |
| | IILANNSLSSNGNVTESGCKECEELEEKNIKEFLQSFVHIVQMFINTS |
| N1D/E64Q | SEQ ID NO: 837 |
| | MSVEHADIWVKSYSLYSRERYICNSGFKRKAGTSSLTECVLNKATNVAHWTTPSLKCIX₁(GGX₂S)₁₋₅DWVNVIS |
| | DLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVQMFINTS |
| | X₁ = K or R; X₂ = G or GG |
| | SEQ ID NO: 1156 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVNVISDLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVQMFINTS |
| N1D/N65D | SEQ ID NO: 838 |
| | MSVEHADIWVKSYSLYSRERYICNSGFKRKAGTSSLTECVLNKATNVAHWTTPSLKCIX₁(GGX₂S)₁₋₅DWVNVIS |
| | DLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1157 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVNVISDLKKIEDLIQSMHIDATLYTESDVHPSCKVTAMKCFLLELQVISLESGDASIHDTVEDL |
| | IILANNSLSSNGNVTESGCKECEELEEKNIKEFLQSFVHIVQMFINTS |
| N-term | SEQ ID NO: 1126 |
| D/N72D | MSVEHADIWVKSYSLYSRERYICNSGFKRKAGTSSLTECVLNKATNVAHWTTPSLKCIX₁(GGX₂S)₁₋₅DNWVNVI |
| | SDLKKIEDLIQSMHIDATLYTESDVHPSCKVTAMKCFLLELQVISLESGDASIHDTVENLIILANDSLSSNGNVT |
| | ESGCKECEELEEKNIKEFLQSFVHIVQMFINTS |
| | X₁ = K or R; X₂ = G or GG |
| | SEQ ID NO: 1158 |
| | MSVEHADIWVKSYSLYSRERYICNSGFKRKAGTSSLTECVLNKATNVAHWTTPSLKCIRGGGGSGGGGSGGGGSG |
| | GGGSGGGGSDNWVNVISDLKKIEDLIQSMHIDATLYTESDVHPSCKVTAMKCFLLELQVISLESGDASIHDTVEN |
| | LIILANDSLSSNGNVTESGCKECEELEEKNIKEFLQSFVHIVQMFINTS |
| N4D/D61N | SEQ ID NO: 839 |
| | MSVEHADIWVKSYSLYSRERYICNSGFKRKAGTSSLTECVLNKATNVAHWTTPSLKCIX₁(GGX₂S)₁₋₅NWVDVIS |
| | DLKKIEDLIQSMHIDATLYTESDVHPSCKVTAMKCFLLELQVISLESGDASIHNTVENLIILANNSLSSNGNVTE |
| | SGCKECEELEEKNIKEFLQSFVHIVQMFINTS |
| | X₁ = K or R; X₂ = G or GG |
| | SEQ ID NO: 1159 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVDVISDLKKIEDLIQSMHIDATLYTESDVHPSCKVTAMKCFLLELQVISLESGDASIHNTVENL |
| | IILANNSLSSNGNVTESGCKECEELEEKNIKEFLQSFVHIVQMFINTS |
| N4D/E64Q | SEQ ID NO: 840 |
| | MSVEHADIWVKSYSLYSRERYICNSGFKRKAGTSSLTECVLNKATNVAHWTTPSLKCIX₁(GGX₂S)₁₋₅NWVDVIS |
| | DLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVQMFINTS |
| | X₁ = K or R; X₂ = G or GG |
| | SEQ ID NO: 1160 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVDVISDLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVQMFINTS |
| N4D/N65D | SEQ ID NO: 841 |
| | MSVEHADIWVKSYSLYSRERYICNSGFKRKAGTSSLTECVLNKATNVAHWTTPSLKCIX₁(GGX₂S)₁₋₅NWVDVIS |
| | DLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1161 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVDVISDLKKIEDLIQSMHIDATLYTESDVHPSCKVTAMKCELLELQVISLESGDASIHDTVEDL |
| | IILANNSLSSNGNVTESGCKECEELEEKNIKEFLQSFVHIVQMFINTS |
| S7G/N65D | SEQ ID NO: 842 |
| | MSVEHADIWVKSYSLYSRERYICNSGFKRKAGTSSLTECVLNKATNVAHWTTPSLKCIX₁(GGX₂S)₁₋₅NWVNVIG |
| | DLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1162 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILANNSLSSNGNVTESGCKECEELEEKNIKEFLQSFVHIVQMFINTS |
| D8G/D61N | SEQ ID NO: 843 |
| | MSVEHADIWVKSYSLYSRERYICNSGFKRKAGTSSLTECVLNKATNVAHWTTPSLKCIX₁(GGX₂S)₁₋₅NWVNVIS |
| | GLKKIEDLIQSMHIDATLYTESDVHPSCKVTAMKCFLLELQVISLESGDASIHNTVENLIILANNSLSSNGNVTE |
| | SGCKECEELEEKNIKEFLQSFVHIVQMFINTS |
| | X₁ = K or R; X₂ = G or GG |
| | SEQ ID NO: 1163 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISGLKKIEDLIQSMHIDATLYTESDVHPSCKVTAMKCFLLELQVISLESGDASIHNTVENL |
| | IILANNSLSSNGNVTESGCKECEELEEKNIKEFLQSFVHIVQMFINTS |
| D8G/E64Q | SEQ ID NO: 844 |
| | MSVEHADIWVKSYSLYSRERYICNSGFKRKAGTSSLTECVLNKATNVAHWTTPSLKCIX₁(GGX₂S)₁₋₅NWVNVIS |
| | GLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVQMFINTS |
| | X₁ = K or R; X₂ = G or GG |
| | SEQ ID NO: 1164 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISGLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVQMFINTS |
| D8G/N65D | SEQ ID NO: 845 |
| | MSVEHADIWVKSYSLYSRERYICNSGFKRKAGTSSLTECVLNKATNVAHWTTPSLKCIX₁(GGX₂S)₁₋₅NWVNVIS |
| | GLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1165 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISGLKKIEDLIQSMHIDATLYTESDVHPSCKVTAMKCFLLELQVISLESGDASIHDTVEDL |
| | IILANNSLSSNGNVTESGCKECEELEEKNIKEFLQSFVHIVQMFINTS |
| D8N/D61N | SEQ ID NO: 846 |
| | MSVEHADIWVKSYSLYSRERYICNSGFKRKAGTSSLTECVLNKATNVAHWTTPSLKCIX₁(GGX₂S)₁₋₅NWVNVIS |
| | NLKKIEDLIQSMHIDATLYTESDVHPSCKVTAMKCFLLELQVISLESGDASIHNTVENLIILANNSLSSNGNVTE |
| | SGCKECEELEEKNIKEFLQSFVHIVQMFINTS |
| | X₁ = K or R; X₂ = G or GG |
| | SEQ ID NO: 1166 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISNLKKIEDLIQSMHIDATLYTESDVHPSCKVTAMKCFLLELQVISLESGDASIHNTVENL |
| | IILANNSLSSNGNVTESGCKECEELEEKNIKEFLQSFVHIVQMFINTS |
| D8N/E64Q | SEQ ID NO: 847 |
| | MSVEHADIWVKSYSLYSRERYICNSGFKRKAGTSSLTECVLNKATNVAHWTTPSLKCIX₁(GGX₂S)₁₋₅NWVNVIS |
| | NLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVQMFINTS |
| | X₁ = K or R; X₂ = G or GG |
| | SEQ ID NO: 1167 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISNLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVQMFINTS |
| D8N/N65D | SEQ ID NO: 848 |
| | MSVEHADIWVKSYSLYSRERYICNSGFKRKAGTSSLTECVLNKATNVAHWTTPSLKCIX₁(GGX₂S)₁₋₅NWVNVIS |
| | NLKKIEDLIQSMHIDATLYTESDVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1168 |
| | MSVEHADIWVKSYSLYSRERYICNSGFKRKAGTSSLTECVLNKATNVAHWTTPSLKCIRGGGGSSGGGGGGGGSG |
| | GGGSGGGGSNWVNVISNLKKIEDLIQSMHIDATLYTESDVHPSCKVTAMKCFLLELQVISLESGDASIHDTVEDL |
| | IILANNSLSSNGNVTESGCKECEELEEKNIKEFLQSFVHIVQMFINTS |
| D30N/N65D | SEQ ID NO: 849 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESNVHPSCKVTAMKCFLLELQVISLESGDASIHDTVEDLIILANNSLSSNGNVTE |
| | SGCKECEELEEKNIKEFLQSFVHIVQMFINTS |
| | X₁ = K or R; X₂ = G or GG |
| | SEQ ID NO: 1169 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESNVHPSCKVTAMKCFLLELQVISLESGDASIHDTVEDL |
| | IILANNSLSSNGNVTESGCKECEELEEKNIKEFLQSFVHIVQMFINTS |
| D61N/E64Q | SEQ ID NO: 850 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHNTVQNLIILANNSLSSNGNVTE |
| | SGCKECEELEEKNIKEFLQSFVHIVQMFINTS |
| | X₁ = K or R; X₂ = G or GG |
| | SEQ ID NO: 1170 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHNTVQNL |
| | IILANNSLSSNGNVTESGCKECEELEEKNIKEFLQSFVHIVQMFINTS |
| D61N/N65D | SEQ ID NO: 851 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCELLELQVISLESGDASIHNTVEDLIILANNSLSSNGNVTE |
| | SGCKECEELEEKNIKEFLQSFVHIVQMFINTS |
| | X₁ = K or R; X₂ = G or GG |
| | SEQ ID NO: 1171 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHNTVEDL |
| | IILANNSLSSNGNVTESGCKECEELEEKNIKEFLQSFVHIVQMFINTS |
| E64Q/N65D | SEQ ID NO: 852 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVQDLIILANNSLSSNGNVTE |
| | SGCKECEELEEKNIKEFLQSFVHIVQMFINTS |
| | X₁ = K or R; X₂ = G or GG |
| | SEQ ID NO: 1172 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVQDL |
| | IILANNSLSSNGNVTESGCKECEELEEKNIKEFLQSFVHIVQMFINTS |
| N65A/I68A | SEQIDNQ:1127 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVEALIALANNSLSSNGNVTE |
| | SGCKECEELEEKNIKEFLQSFVHIVQMFINTS |
| | X₁ = K or R; X₂ = G or GG |
| | SEQ ID NO: 1173 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCELLELQVISLESGDASIHDTVEAL |
| | IALANNSLSSNGNVTESGCKECEELEEKNIKEFLQSFVHIVQMFINTS |
| N65D/I68A | SEQ ID NO: 853 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVEDLIALANNSLSSNGNVTE |
| | SGCKECEELEEKNIKEFLQSFVHIVQMFINTS |
| | X₁ = K or R; X₂ = G or GG |
| | SEQ ID NO: 1174 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVEDL |
| | IALANNSLSSNGNVTESGCKECEELEEKNIKEFLQSFVHIVQMFINTS |
| E64Q/Q108E | SEQ ID NO: 854 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVQNLIILANNSLSSNGNVTE |
| | SGCKECEELEEKNIKEFLQSFVHIVEMFINTS |
| | X₁ = K or R; X₂ = G or GG |
| | SEQ ID NO: 1175 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVQNL |
| | IILANNSLSSNGNVTESGCKECEELEEKNIKEFLQSFVHIVEMFINTS |
| D30N/E64Q/ | SEQ ID NO: 855 |
| N65D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESNVHPSCKVTAMKCFLLELQVISLESGDASIHDTVQDLIILANNSLSSNGNVTE |
| | SGCKECEELEEKNIKEFLQSFVHIVQMFINTS |
| | X₁ = K or R; X₂ = G or GG |
| | SEQ ID NO: 1176 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESNVHPSCKVTAMKCFLLELQVISLESGDASIHDTVQDL |
| | IILANNSLSSNGNVTESGCKECEELEEKNIKEFLQSFVHIVQMFINTS |
| D61N/E64Q/ | SEQ ID NO: 856 |
| N65D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHNTVQDLIILANNSLSSNGNVTE |
| | SGCKECEELEEKNIKEFLQSFVHIVQMFINTS |
| | X1 = K or R; X2 = G or GG |
| | SEQ ID NO: 1177 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHNTVQDL |
| | IILANNSLSSNGNVTESGCKECEELEEKNIKEFLQSFVHIVQMFINTS |
| D61N/N65D/ | SEQ ID NO: 857 |
| I68A | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHNTVEDLIALANNSLSSNGNVTE |
| | SGCKECEELEEKNIKEFLQSFVHIVQMFINTS |
| | X₁ = K or R; X₂ = G or GG |
| | SEQ ID NO: 1178 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHNTVEDL |
| | IALANNSLSSNGNVTESGCKECEELEEKNIKEFLQSFVHIVQMFINTS |
| N71L/N79P | SEQ ID NO: 858 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSNGPVTE |
| | SGCKECEELEEKNIKEFLQSFVHIVQMFINTS |
| | X₁ = K or R; X₂ = G or GG |
| | SEQ ID NO: 1179 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILALNSLSSNGPVTESGCKECEELEEKNIKEFLQSFVHIVQMFINTS |
| N71L/N112D | SEQ ID NO: 859 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSNGNVTE |
| | SGCKECEELEEKNIKEFLQSFVHIVQMFIDTS |
| | X₁ = K or R; X₂ = G or GG |
| | SEQ ID NO: 1180 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILALNSLSSNGNVTESGCKECEELEEKNIKEFLQSFVHIVQMFIDTS |
| N79P/N112D | SEQ ID NO: 860 |
| | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILANNSLSSNGPVTE |
| | SGCKECEELEEKNIKEFLQSFVHIVQMFIDTS |
| | X₁ = K or R; X₂ = G or GG |
| | SEQ ID NO: 1181 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILANNSLSSNGPVTESGCKECEELEEKNIKEFLQSFVHIVQMFIDTS |
| S7G/N65A/ | SEQ ID NO: 861 |
| I68A | MSVEHADIWVKSYSLYSRERYICNSGFKRKAGTSSLTECVLNKATNVAHWTTPSLKCIX₁(GGX₂S)₁₋₅NWVNVIG |
| | DLKKIEDLIQSMHIDATLYTESDVHPSCKVTAMKCFLLELQVISLESGDASIHDTVEALIALANNSLSSNGNVTE |
| | SGCKECEELEEKNIKEFLQSFVHIVQMFINTS |
| | X₁ = K or R; X₂ = G or GG |
| | SEQ ID NO: 1182 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCELLELQVISLESGDASIHDTVEAL |
| | IALANNSLSSNGNVTESGCKECEELEEKNIKEFLQSFVHIVQMFINTS |
| S7G/N65D/ | SEQ ID NO: 862 |
| I68A | MSVEHADIWVKSYSLYSRERYICNSGFKRKAGTSSLTECVLNKATNVAHWTTPSLKCIX₁(GGX₂S)₁₋₅NWVNVIG |
| | DLKKIEDLIQSMHIDATLYTESDVHPSCKVTAMKCFLLELQVISLESGDASIHDTVEDLIALANNSLSSNGNVTE |
| | SGCKECEELEEKNIKEFLQSFVHIVQMFINTS |
| | X₁ = K or R; X2 = G or GG |
| | SEQ ID NO: 1183 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IALANNSLSSNGNVTESGCKECEELEEKNIKEFLQSFVHIVQMFINTS |
| N71Q/N79Q/ | SEQ ID NO: 863 |
| N112Q | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAQNSLSSNGQVTE |
| | SGCKECEELEEKNIKEFLQSFVHIVQMFIQTS |
| | X₁ = K or R; X₂ = G or GG |
| | SEQ ID NO: 1184 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAQNSLSSNGQVTESGCKECEELEEKNIKEFLQSFVHIVQMFIQTS |
| N71A/N79A/ | SEQ ID NO: 864 |
| N112A | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAANSLSSNGAVTE |
| | SGCKECEELEEKNIKEFLQSFVHIVQMFIATS |
| | X₁ = K or R; X₂ = G or GG |
| | SEQ ID NO: 1185 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAANSLSSNGAVTESGCKECEELEEKNIKEFLQSFVHIVQMFIATS |
| N71G/N79G/ | SEQ ID NO: 865 |
| N112G | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAGNSLSSNGGVTE |
| | SGCKECEELEEKNIKEFLQSFVHIVQMFIGTS |
| | X₁ = K or R; X₂ = G or GG |
| | SEQ ID NO: 1186 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAGNSLSSNGGVTESGCKECEELEEKNIKEFLQSFVHIVQMFIGTS |
| N71S/N79L/ | SEQ ID NO: 866 |
| N112E | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILASNSLSSNGLVTE |
| | SGCKECEELEEKNIKEFLQSFVHIVQMFIETS |
| | X₁ = K or R; X₂ = G or GG |
| | SEQ ID NO: 1187 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILASNSLSSNGLVTESGCKECEELEEKNIKEFLQSFVHIVQMFIETS |
| N71E/N79P/ | SEQ ID NO: 867 |
| N112K | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSNGPVTE |
| | SGCKECEELEEKNIKEFLQSFVHIVQMFIKTS |
| | X₁ = K or R; X₂ = G or GG |
| | SEQ ID NO: 1188 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSNGPVTESGCKECEELEEKNIKEFLQSFVHIVQMFIKTS |
| N71L/N79P/ | SEQ ID NO: 868 |
| N112K | MSVEHADIWVKSYSLYSRERYICNSGFKRKAGTSSLTECVLNKAINVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSNGPVTE |
| | SGCKECEELEEKNIKEFLQSFVHIVQMFIKTS |
| | X₁ = K or R; X₂ = G or GG |
| | SEQ ID NO: 1189 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILALNSLSSNGPVTESGCKECEELEEKNIKEFLQSFVHIVQMFIKTS |
| N71L/N79P/ | SEQ ID NO: 869 |
| N112D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSNGPVTE |
| | SGCKECEELEEKNIKEFLQSFVHIVQMFIDTS |
| | X₁ = K or R; X₂ = G or GG |
| | SEQ ID NO: 1190 |
| | MSVEHADIWVKSYSLYSRERYICNSGFKRKAGTSSLTECVLNKATNVAHWTTPSLKCIRGGGGSSGGGGGGGGSG |
| | GGGSGGGGSNWVNVISDLKKIEDLIQSMHIDATLYTESDVHPSCKVTAMKCFLLELQVISLESGDASIHDTVENL |
| | IILALNSLSSNGPVTESGCKECEELEEKNIKEFLQSFVHIVQMFIDTS |
| N71K/N79P/ | SEQ ID NO: 870 |
| N112D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAKNSLSSNGPVTE |
| | SGCKECEELEEKNIKEFLQSFVHIVQMFIDTS |
| | X₁ = K or R; X₂ = G or GG |
| | SEQ ID NO: 1191 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAKNSLSSNGPVTESGCKECEELEEKNIKEFLQSFVHIVQMFIDTS |
| N71E/N79P/ | SEQ ID NO: 871 |
| N112S | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSNGPVTE |
| | SGCKECEELEEKNIKEFLQSFVHIVQMFISTS |
| | X₁ = K or R; X₂ = G or GG |
| | SEQ ID NO: 1192 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSNGPVTESGCKECEELEEKNIKEFLQSFVHIVQMFISTS |
| N71E/N79P/ | SEQ ID NO: 872 |
| N112D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSNGPVTE |
| | SGCKECEELEEKNIKEFLQSFVHIVQMFIDTS |
| | X₁ = K or R; X₂ = G or GG |
| | SEQ ID NO: 1193 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSNGPVTESGCKECEELEEKNIKEFLQSFVHIVQMFIDTS |
| N1D/D61N/ | SEQ ID NO: 873 |
| E64Q/Q108E | MSVEHADIWVKSYSLYSRERYICNSGFKRKAGTSSLTECVLNKATNVAHWTTPSLKCIX₁(GGX₂S)₁₋₅DWVNVIS |
| | DLKKIEDLIQSMHIDATLYTESDVHPSCKVTAMKCFLLELQVISLESGDASIHNTVQNLIILANNSLSSNGNVTE |
| | SGCKECEELEEKNIKEFLQSFVHIVEMFINTS |
| | X₁ = K or R; X₂ = G or GG |
| | SEQ ID NO: 1194 |
| | MSVEHADIWVKSYSLYSRERYICNSGFKRKAGTSSLTECVLNKATNVAHWTTPSLKCIRGGGGSGGGGSGGGGSG |
| | GGGSGGGGSDWVNVISDLKKIEDLIQSMHIDATLYTESDVHPSCKVTAMKCFLLELQVISLESGDASIHNTVQNL |
| | IILANNSLSSNGNVTESGCKECEELEEKNIKEFLQSFVHIVEMFINTS |
| N4D/D61N/ | SEQ ID NO: 874 |
| E64Q/Q108E | MSVEHADIWVKSYSLYSRERYICNSGFKRKAGTSSLTECVLNKATNVAHWTTPSLKCIX₁(GGX₂S)₁₋₅NWVDVIS |
| | DLKKIEDLIQSMHIDATLYTESDVHPSCKVTAMKCFLLELQVISLESGDASIHNTVQNLIILANNSLSSNGNVTE |
| | SGCKECEELEEKNIKEFLQSFVHIVEMFINTS |
| | X₁ = K or R; X₂ = G or GG |
| | SEQ ID NO: 1195 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVDVISDLKKIEDLIQSMHIDATLYTESDVHPSCKVTAMKCFLLELQVISLESGDASIHNTVQNL |
| | IILANNSLSSNGNVTESGCKECEELEEKNIKEFLQSFVHIVEMFINTS |
| N71Q/N77L/ | SEQ ID NO: 875 |
| N79Q/N112Q | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCELLELQVISLESGDASIHDTVENLIILAQNSLSSLGQVTE |
| | SGCKECEELEEKNIKEFLQSFVHIVQMFIQTS |
| | X₁ = K or R; X₂ = G or GG |
| | SEQ ID NO: 1196 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAQNSLSSLGQVTESGCKECEELEEKNIKEFLQSFVHIVQMFIQTS |
| N71A/N77L/ | SEQ ID NO: 876 |
| N79A/N112A | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAANSLSSLGAVTE |
| | SGCKECEELEEKNIKEFLQSFVHIVQMFIATS |
| | X₁ = K or R; X₂ = G or GG |
| | SEQ ID NO: 1197 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAANSLSSLGAVTESGCKECEELEEKNIKEFLQSFVHIVQMFIATS |
| N71G/N77L/ | SEQ ID NO: 877 |
| N79G/N112G | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAGNSLSSLGGVTE |
| | SGCKECEELEEKNIKEFLQSFVHIVQMFIGTS |
| | X₁ = K or R; X₂ = G or GG |
| | SEQ ID NO: 1198 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAGNSLSSLGGVTESGCKECEELEEKNIKEFLQSFVHIVQMFIGTS |
| N71S/N77L/ | SEQ ID NO: 878 |
| N79L/N112E | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILASNSLSSLGLVTE |
| | SGCKECEELEEKNIKEFLQSFVHIVQMFIETS |
| | X₁ = K or R; X₂ = G or GG |
| | SEQ ID NO: 1199 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILASNSLSSLGLVTESGCKECEELEEKNIKEFLQSFVHIVQMFIETS |
| N71E/N77L/ | SEQ ID NO: 879 |
| N79P/N112K | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSLGPVTE |
| | SGCKECEELEEKNIKEFLQSFVHIVQMFIKTS |
| | X₁ = K or R; X₂ = G or GG |
| | SEQ ID NO: 1200 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSLGPVTESGCKECEELEEKNIKEFLQSFVHIVQMFIKTS |
| N71L/N77L/ | SEQ ID NO: 880 |
| N79P/N112K | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILALNSLSSLGPVTE |
| | SGCKECEELEEKNIKEFLQSFVHIVQMFIKTS |
| | X₁ = K or R; X₂ = G or GG |
| | SEQ ID NO: 1201 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILALNSLSSLGPVTESGCKECEELEEKNIKEFLQSFVHIVQMFIKTS |
| N71E/N77L/ | SEQ ID NO: 881 |
| N79P/N112S | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSLGPVTE |
| | SGCKECEELEEKNIKEFLQSFVHIVQMFISTS |
| | X₁ = K or R; X₂ = G or GG |
| | SEQ ID NO: 1202 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSLGPVTESGCKECEELEEKNIKEFLQSFVHIVQMFISTS |
| N71E/N77L/ | SEQ ID NO: 882 |
| N79P/N112D | MSVEHADIWVKSYSLYSRERYICNSGFKRKAGTSSLTECVLNKATNVAHWTTPSLKCIX₁(GGX₂S)₁₋₅NWVNVIS |
| | DLKKIEDLIQSMHIDATLYTESDVHPSCKVTAMKCFLLELQVISLESGDASIHDTVENLIILAENSLSSLGPVTE |
| | SGCKECEELEEKNIKEFLQSFVHIVQMFIDTS |
| | X₁ = K or R; X₂ = G or GG |
| | SEQ ID NO: 1203 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVISDLKKIEDLIQSMHIDATLYTESDVHPSCKVTAMKCFLLELQVISLESGDASIHDTVENL |
| | IILAENSLSSLGPVTESGCKECEELEEKNIKEFLQSFVHIVQMFIDTS |
| S7G/N65D/ | SEQ ID NO: 883 |
| N71L/N79P/ | MSVEHADIWVKSYSLYSRERYICNSGFKRKAGTSSLTECVLNKATNVAHWTTPSLKCIX₁(GGX₂S)₁₋₅NWVNVIG |
| N112D | DLKKIEDLIQSMHIDATLYTESDVHPSCKVTAMKCFLLELQVISLESGDASIHDTVEDLIILALNSLSSNGPVTE |
| | SGCKECEELEEKNIKEFLQSFVHIVQMFIDTS |
| | X₁ = K or R; X₂ = G or GG |
| | SEQ ID NO: 1204 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILALNSLSSNGPVTESGCKECEELEEKNIKEFLQSFVHIVQMFIDTS |
| S7G/N65D/ | SEQ ID NO: 884 |
| N71E/N77L/ | MSVEHADIWVKSYSLYSRERYICNSGFKRKAGTSSLTECVLNKATNVAHWTTPSLKCIX₁(GGX₂S)₁₋₅NWVNVIG |
| N79P/N112D | DLKKIEDLIQSMHIDATLYTESDVHPSCKVTAMKCFLLELQVISLESGDASIHDTVEDLIILAENSLSSLGPVTE |
| | SGCKECEELEEKNIKEFLQSFVHIVQMFIDTS |
| | X₁ = K or R; X₂ = G or GG |
| | SEQ ID NO: 1205 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILAENSLSSLGPVTESGCKECEELEEKNIKEFLQSFVHIVQMFIDTS |
| S7G/N65D/ | SEQ ID NO: 885 |
| N71E/N77L/ | MSVEHADIWVKSYSLYSRERYICNSGFKRKAGTSSLTECVLNKATNVAHWTTPSLKCIX₁(GGX₂S)₁₋₅NWVNVIG |
| N79P/N112S | DLKKIEDLIQSMHIDATLYTESDVHPSCKVTAMKCFLLELQVISLESGDASIHDTVEDLIILAENSLSSLGPVTE |
| | SGCKECEELEEKNIKEFLQSFVHIVQMFISTS |
| | X₁ = K or R; X₂ = G or GG |
| | SEQ ID NO: 1206 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILAENSLSSLGPVTESGCKECEELEEKNIKEFLQSFVHIVQMFISTS |
| S7G/N65D/ | SEQ ID NO: 886 |
| N71L/N77L/ | MSVEHADIWVKSYSLYSRERYICNSGFKRKAGTSSLTECVLNKATNVAHWTTPSLKCIX₁(GGX₂S)₁₋₅NWVNVIG |
| N79P/N112K | DLKKIEDLIQSMHIDATLYTESDVHPSCKVTAMKCFLLELQVISLESGDASIHDTVEDLIILALNSLSSLGPVTE |
| | SGCKECEELEEKNIKEFLQSFVHIVQMFIKTS |
| | X₁ = K or R; X₂ = G or GG |
| | SEQ ID NO: 1207 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILALNSLSSLGPVTESGCKECEELEEKNIKEFLQSFVHIVQMFIKTS |
| S7G/N65D/ | SEQ ID NO: 887 |
| N71E/N77L/ | MSVEHADIWVKSYSLYSRERYICNSGFKRKAGTSSLTECVLNKATNVAHWTTPSLKCIX₁(GGX₂S)₁₋₅NWVNVIG |
| N79P/N112K | DLKKIEDLIQSMHIDATLYTESDVHPSCKVTAMKCFLLELQVISLESGDASIHDTVEDLIILAENSLSSLGPVTE |
| | SGCKECEELEEKNIKEFLQSFVHIVQMFIKTS |
| | X₁ = K or R; X₂ = G or GG |
| | SEQ ID NO: 1208 |
| | MSVEHADIWVKSYSLYSRERYICNSGFKRKAGTSSLTECVLNKATNVAHWTTPSLKCIRGGGGSGGGGSGGGGSG |
| | GGGSGGGGSNWVNVIGDLKKIEDLIQSMHIDATLYTESDVHPSCKVTAMKCFLLELQVISLESGDASIHDTVEDL |
| | IILAENSLSSLGPVTESGCKECEELEEKNIKEFLQSFVHIVQMFIKTS |

### 4. CD4-Targeted IL-15v Molecules

Provided are CD4-binding IL-15v molecules.

a. Formats

In various embodiments, the CD4-binding IL-15v molecules can have three polypeptide chains or four polypeptide chains.

### Three Polypeptide Chain Format

In some embodiments, provided is a CD4-targeted interleukin-15 (IL-15) molecule comprising: (a) a first polypeptide comprising an immunoglobulin heavy chain comprising a first immunoglobulin fragment crystallizable domain (Fc domain); (b) a second polypeptide comprising an immunoglobulin light chain (VL-CL), wherein the first polypeptide and the second polypeptide form an antigen binding domain that specifically binds to CD4; and (c) a third polypeptide comprising a fusion protein comprising, e.g., from the N-terminus to the C-terminus: (i) an IL-15 receptor alpha subunit (IL15RA) SUSHI domain, (ii) an IL-15 or variant thereof (IL-15v), and (iii) a second Fc domain, wherein the first Fc domain and the second Fc domain heterodimerize to form a bispecific molecule that binds to CD4 and a IL-2βγ complex (CD122 and CD132). In some embodiments, the antigen binding domain that specifically binds to CD4 is a Fab that does not have any non-canonical disulfide bonds. Illustrative CD4-targeted IL-15v having three polypeptide chains are provided in Table G, e.g., molecules 100-222. In the CD4-targeted IL-15v provided in Table G, the immunoglobulin heavy chain and the immunoglobulin light chain (VL-CL) form a Fab that does not have any non-canonical disulfide bonds.

### Four Polypeptide Chain Format

In some embodiments, provided is a CD4-targeted interleukin-15 (IL-15) molecule comprising: (a) first and second immunoglobulin heavy chains, comprising first and second immunoglobulin Fc domains, wherein the first and second Fc domains heterodimerize to form a bispecific molecule; (b) first and second immunoglobulin light chains (VL-CL), wherein the first and second immunoglobulin heavy chains and the first and second immunoglobulin light chains form first and second antigen binding domains that specifically bind to CD4, respectively; and (c) a fusion protein comprising, e.g., from the N-terminus to the C-terminus: (i) an IL-15 receptor alpha subunit (IL15RA) SUSHI domain, (ii) an IL-15 or variant thereof (IL-15v), wherein the fusion protein is linked or fused to the C-terminus of one or both of the first and second Fc domains; wherein the bispecific molecule binds to CD4 and a IL-2βγ complex (CD122 and CD132).). In some embodiments, the antigen binding domain that specifically binds to CD4 is a Fab that does not have any non-canonical disulfide bonds. Illustrative CD4-targeted IL-15v having four polypeptide chains are provided in Table G, e.g., molecules 223-247.

b. CD4-Binding Domains

The herein described CD4-binding IL-15v molecules have CD4 binding domains. In some embodiments, the CD4-binding domains are immunoglobulin binding domains, e.g., a heavy chain variable region (VH) and a light chain variable region (VL). In some embodiments, the CD4 binding domain is in a Fab format. In some embodiments, the antigen binding domain that specifically binds to CD4 binds to the CD4 D1 domain, the CD4 D2 domain, one or both of the CD4 D2 domain and the CD4 D3 domain, or the CD4 D3 domain. The CDR and VH/VL amino acid sequences of illustrative CD4 binding domains that can be used in the herein described CD4-targeted IL-15v are provided in Tables A1, A2, A3, A4 and Table B.

In some embodiments, the antigen binding domain that specifically binds to CD4 competes with or comprises a heavy chain variable region (VH) and a light chain variable region (VL) from an antibody selected from the group consisting of ibalizumab, tregalizumab, keliximab, zanolimumab, clenoliximab, priliximab, UB-421, RPA-T4, SK3, MEM241 and OKT-4.

CD4 Binding Domains that Bind to CD4 D1

In some embodiments, the antigen binding domain that specifically binds to CD4 specifically binds to the CD4 D1 domain. In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of:
1) SEQ ID NOs: 1, 2, 3, 4, 5 and 6; (1)
2) SEQ ID NOs: 1, 7, 3, 4, 8 and 9; (2)
3) SEQ ID NOs: 1, 10, 3, 11, 5 and 6; (3)
4) SEQ ID NOs: 1, 10, 3, 11, 12 and 6; (4)
5) SEQ ID NOs: 1, 10, 3, 11, 8 and 9; (5)
6) SEQ ID NOs: 1, 13, 3, 11, 8 and 9; (6)
7) SEQ ID NOs: 1, 14, 3, 11, 8 and 9; (7)
8) SEQ ID NOs: 1, 15, 3, 11, 8 and 9; (8)
9) SEQ ID NOs: 1, 16, 3, 11, 8 and 9; (9)
10) SEQ ID NOs: 1, 15, 3, 11, 12 and 17; (10)
11) SEQ ID NOs: 1, 16, 3, 11, 12 and 18; (11)
12) SEQ ID NOs: 1, 1078, 3, 11, 8 and 9; (12)
13) SEQ ID NOs: 1, 1079, 3, 11, 8 and 9; (13, 14, 17, 18 and 19)
14) SEQ ID NOs: 1, 1080, 3, 11, 8 and 9; (15)
15) SEQ ID NOs: 1, 1081, 3, 11, 8 and 9; (16)
16) SEQ ID NOs: 19, 20, 21, 22, 23 and 24; (1.17)
17) SEQ ID NOs: 25, 26, 27, 28, 29 and 30; (1.25)
18) SEQ ID NOs: 31, 32, 33, 34, 29 and 35; (1.26)
19) SEQ ID NOs: 25, 36, 37, 28, 29 and 38; (1.27)
20) SEQ ID NOs: 25, 1082, 39, 28, 29 and 38; (1.28)
21) SEQ ID NOs: 40, 41, 42, 28, 29 and 35; (1.29)
22) SEQ ID NOs: 25, 43, 39, 28, 29 and 38; (1.30)
23) SEQ ID NOs: 25, 44, 45, 28, 29 and 35; (1.31)
24) SEQ ID NOs: 25, 46, 47, 28, 29 and 35; (1.32)
25) SEQ ID NOs: 25, 48, 49, 28, 29 and 35; (1.33)
26) SEQ ID NOs: 25, 48, 50, 28, 29 and 35; (1.34)
27) SEQ ID NOs: 51, 52, 53, 54, 55 and 56; (1.45)
28) SEQ ID NOs: 51, 57, 53, 58, 55 and 56; (1.46, 1.47, 1.48)
29) SEQ ID NOs: 59, 60, 61, 62, 63 and 64; (keliximab) or
30) SEQ ID NOs: 65, 66, 67, 68, 69 and 70; (UB-421).

In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of:
1) SEQ ID NOs: 178, 179, 180, 181, 182 and 6; (1)
2) SEQ ID NOs: 178, 183, 180, 181, 182 and 9; (2)
3) SEQ ID NOs: 178, 184, 180, 181, 182 and 6; (4)
4) SEQ ID NOs: 178, 184, 180, 181, 182 and 9; (3, 5)
5) SEQ ID NOs: 178, 185, 180, 181, 182 and 9; (6, 7, 12)
6) SEQ ID NOs: 178, 186, 180, 181, 182 and 9; (8)
7) SEQ ID NOs: 178, 187, 180, 181, 182 and 9; (9)
8) SEQ ID NOs: 178, 186, 180, 181, 182 and 17; (10)
9) SEQ ID NOs: 178, 187, 180, 181, 182 and 18; (11)
10) SEQ ID NOs: 178, 188, 180, 181, 182 and 9; (13, 14, 17, 18, 19)
11) SEQ ID NOs: 178, 189, 180, 181, 182 and 9; (15)
12) SEQ ID NOs: 178, 190, 180, 181, 182 and 9; (16)
13) SEQ ID NOs: 191, 192, 193, 194, 195 and 24; (1.17)
14) SEQ ID NOs: 196, 197, 198, 199, 200 and 30; (1.25)
15) SEQ ID NOs: 201, 202, 203, 199, 200 and 35; (1.26)
16) SEQ ID NOs: 204, 205, 206, 199, 200 and 38; (1.27)
17) SEQ ID NOs: 196, 207, 208, 199, 200 and 38; (1.28)
18) SEQ ID NOs: 209, 210, 211, 199, 200 and 35; (1.29)
19) SEQ ID NOs: 196, 212, 208, 199, 200 and 38; (1.30)
20) SEQ ID NOs: 196, 213, 214, 199, 200 and 35; (1.31)
21) SEQ ID NOs: 196, 213, 215, 199, 200 and 35; (1.32)
22) SEQ ID NOs: 196, 216, 217, 199, 200 and 35; (1.33)
23) SEQ ID NOs: 196, 216, 218, 199, 200 and 35; (1.34)
24) SEQ ID NOs: 219, 220, 221, 222, 223 and 56; (1.45)
25) SEQ ID NOs: 219, 224, 221, 225, 223 and 56; (1.46, 1.47, 1.48)
26) SEQ ID NOs: 226, 227, 228, 229, 230 and 64; (keliximab) or
27) SEQ ID NOs: 231, 232, 233, 234, 235 and 70; (UB-421).

In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of:
1) SEQ ID NOs: 323, 324, 325, 326, 182 and 327; (1)
2) SEQ ID NOs: 323, 328, 325, 326, 182 and 329; (2)
3) SEQ ID NOs: 323, 330, 325, 326, 182 and 327; (4)
4) SEQ ID NOs: 323, 330, 325, 326, 182 and 329; (5)
5) SEQ ID NOs: 323, 331, 325, 326, 182 and 329; (6, 7, 12)
6) SEQ ID NOs: 323, 332, 325, 326, 182 and 329; (8)
7) SEQ ID NOs: 323, 333, 325, 326, 182 and 329; (9)
8) SEQ ID NOs: 323, 332, 325, 326, 182 and 334; (10)
9) SEQ ID NOs: 323, 333, 325, 326, 182 and 335; (11)
10) SEQ ID NOs: 323, 336, 325, 326, 182 and 329; (13, 14, 17, 18 and 19)
11) SEQ ID NOs: 323, 337, 325, 326, 182 and 329; (15)
12) SEQ ID NOs: 323, 338, 325, 326, 182 and 329; (16)
13) SEQ ID NOs: 339, 340, 341, 342, 195 and 1131; (1.17)
14) SEQ ID NOs: 344, 345, 346, 347, 200 and 348; (1.25)
15) SEQ ID NOs: 349, 350, 351, 347, 200 and 352; (1.26)
16) SEQ ID NOs: 353, 354, 355, 347, 200 and 356; (1.27)
17) SEQ ID NOs: 344, 350, 357, 347, 200 and 356; (1.28)
18) SEQ ID NOs: 358, 350, 359, 347, 200 and 352; (1.29)
19) SEQ ID NOs: 343, 354, 357, 347, 200 and 356; (1.30)
20) SEQ ID NOs: 343, 360, 361, 347, 200 and 352; (1.31)
21) SEQ ID NOs: 343, 360, 362, 347, 200 and 352; (1.32)
22) SEQ ID NOs: 343, 363, 364, 347, 200 and 352; (1.33)
23) SEQ ID NOs: 343, 363, 365, 347, 200 and 352; (1.34)
24) SEQ ID NOs: 366, 367, 368, 1084, 223 and 369; (1.45)
25) SEQ ID NOs: 366, 367, 368, 370, 223 and 369; (1.46, 1.47, 1.48)
26) SEQ ID NOs: 371, 372, 373, 374, 230 and 375; (keliximab) or
27) SEQ ID NOs: 376, 377, 378, 379, 235 and 380; (UB-421).

In some embodiments, the antigen binding domain specifically binds to CD4 D1 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of:
1) SEQ ID NOs: 463, 464, 465, 466, 467 and 327; (1)
2) SEQ ID NOs: 463, 468, 465, 466, 469 and 329; (2)
3) SEQ ID NOs: 463, 470, 465, 466, 467 and 327; (3)
4) SEQ ID NOs: 463, 470, 465, 466, 469 and 327; (4)
5) SEQ ID NOs: 463, 470, 465, 466, 469 and 329; (5)
6) SEQ ID NOs: 463, 1132, 465, 466, 469 and 329; (6)
7) SEQ ID NOs: 463, 471, 465, 466, 469 and 329; (7)
8) SEQ ID NOs: 463, 472, 465, 466, 469 and 329; (8)
9) SEQ ID NOs: 463, 473, 465, 466, 469 and 329; (9)
10) SEQ ID NOs: 463, 472, 465, 466, 474 and 334; (10)
11) SEQ ID NOs: 463, 473, 465, 466, 474 and 335; (11)
12) SEQ ID NOs: 463, 475, 465, 466, 469 and 329; (12)
13) SEQ ID NOs: 463, 478, 465, 466, 469 and 329; (13, 14, 17, 18 and 19)
14) SEQ ID NOs: 463, 476, 465, 466, 469 and 329; (15)
15) SEQ ID NOs: 463, 477, 465, 466, 469 and 329; (16)
16) SEQ ID NOs: 479, 480, 481, 482, 483 and 1131; (1.17)
17) SEQ ID NOs: 484, 485, 486, 487, 488 and 348; (1.25)
18) SEQ ID NOs: 489, 490, 491, 487, 492 and 352; (1.26)
19) SEQ ID NOs: 493, 494, 495, 487, 492 and 356; (1.27)
20) SEQ ID NOs: 484, 496, 497, 487, 492 and 356; (1.28)
21) SEQ ID NOs: 498, 499, 500, 487, 492 and 352; (1.29)
22) SEQ ID NOs: 484, 501, 497, 487, 492 and 356; (1.30)
23) SEQ ID NOs: 484, 502, 503, 487, 492 and 352; (1.31)
24) SEQ ID NOs: 484, 504, 505, 487, 506 and 352; (1.32)
25) SEQ ID NOs: 484, 507, 508, 487, 492 and 352; (1.33)
26) SEQ ID NOs: 484, 507, 509, 487, 492 and 352; (1.34)
27) SEQ ID NOs: 510, 511, 512, 513, 514 and 369; (1.45)
28) SEQ ID NOs: 510, 515, 512, 516, 514 and 369; (1.46, 1.47, 1.48)
29) SEQ ID NOs: 517, 518, 519, 374, 520 and 375; (keliximab) or
30) SEQ ID NOs: 521, 522, 523, 524, 525 and 380; (UB-421).

In some embodiments, the antigen binding domain specifically binds to CD4 D1 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 625 and 626; (1)
2) SEQ ID NOs: 627 and 628; (2)
3) SEQ ID NOs: 629 and 630; (3)
4) SEQ ID NOs: 629 and 631; (4)
5) SEQ ID NOs: 629 and 632; (5)
6) SEQ ID NOs: 633 and 632; (6)
7) SEQ ID NOs: 634 and 632; (7)
8) SEQ ID NOs: 635 and 632; (8)
9) SEQ ID NOs: 636 and 632; (9)
10) SEQ ID NOs: 635 and 637; (10)
11) SEQ ID NOs: 636 and 638; (11)
12) SEQ ID NOs: 639 and 632; (12)
13) SEQ ID NOs: 640 and 632; (13)
14) SEQ ID NOs: 641 and 632; (14)
15) SEQ ID NOs: 642 and 632; (15)
16) SEQ ID NOs: 643 and 632; (16)
17) SEQ ID NOs: 640 and 644; (17)
18) SEQ ID NOs: 640 and 645; (18)
19) SEQ ID NOs: 640 and 646; (19)
20) SEQ ID NOs: 647 and 648; (1.17)
21) SEQ ID NOs: 649 and 650; (1.25)
22) SEQ ID NOs: 651 and 652; (1.26)
23) SEQ ID NOs: 653 and 654; (1.27)
24) SEQ ID NOs: 655 and 656; (1.28)
25) SEQ ID NOs: 657 and 658; (1.29)
26) SEQ ID NOs: 659 and 660; (1.30)
27) SEQ ID NOs: 661 and 662; (1.31)
28) SEQ ID NOs: 663 and 664; (1.32)
29) SEQ ID NOs: 665 and 666; (1.33)
30) SEQ ID NOs: 667 and 666; (1.34)
31) SEQ ID NOs: 668 and 669; (1.45)
32) SEQ ID NOs: 670 and 671; (1.46)
33) SEQ ID NOs: 672 and 673; (1.47)
34) SEQ ID NOs: 672 and 671; (1.48)
35) SEQ ID NOs: 674 and 675; (keliximab) or
36) SEQ ID NOs: 676 and 677; (UB-421).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 2, 3, 4, 5 and 6 (according to Kabat);
2) SEQ ID NOs: 178, 179, 180, 181, 182 and 6 (according to IMGT);
3) SEQ ID NOs: 323, 324, 325, 326, 182 and 327 (according to Chothia); or
4) SEQ ID NOs: 463, 464, 465, 466, 467 and 327 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 625 and 626 (antibody 1).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 7, 3, 4, 8 and 9 (according to Kabat);
2) SEQ ID NOs: 178, 183, 180, 181, 182 and 9 (according to IMGT);
3) SEQ ID NOs: 323, 328, 325, 326, 182 and 329 (according to Chothia); or
4) SEQ ID NOs: 463, 468, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 627 and 628 (antibody 2).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 5 and 6 (according to Kabat);
2) SEQ ID NOs: 178, 184, 180, 181, 182 and 9 (according to IMGT);
3) SEQ ID NOs: 323, 330, 325, 326, 182 and 327 (according to Chothia); or
4) SEQ ID NOs: 463, 470, 465, 466, 467 and 327 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 630 (antibody 3).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 12 and 6 (according to Kabat);
2) SEQ ID NOs: 178, 184, 180, 181, 182 and 6 (according to IMGT);
3) SEQ ID NOs: 323, 330, 325, 326, 182 and 327 (according to Chothia); or
4) SEQ ID NOs: 463, 470, 465, 466, 469 and 327 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 631 (antibody 4).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 10, 3, 11, 8 and 9 (according to Kabat);
2) SEQ ID NOs: 178, 184, 180, 181, 182 and 9 (according to IMGT);
3) SEQ ID NOs: 323, 330, 325, 326, 182 and 329 (according to Chothia); or
4) SEQ ID NOs: 463, 470, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 629 and 632 (antibody 5).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 13, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 1132, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 633 and 632 (antibody 6).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 14, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 471, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 634 and 632 (antibody 7).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 15, 3, 11, 8 and 9 (according to Kabat);
2) SEQ ID NOs: 178, 186, 180, 181, 182 and 9 (according to IMGT);
3) SEQ ID NOs: 323, 332, 325, 326, 182 and 329 (according to Chothia); or
4) SEQ ID NOs: 463, 472, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 635 and 632 (antibody 8).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 16, 3, 11, 8 and 9 (according to Kabat);
2) SEQ ID NOs: 178, 187, 180, 181, 182 and 9 (according to IMGT);
3) SEQ ID NOs: 323, 333, 325, 326, 182 and 329 (according to Chothia); or
4) SEQ ID NOs: 463, 473, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 636 and 632 (antibody 9).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 15, 3, 11, 12 and 17 (according to Kabat);
2) SEQ ID NOs: 178, 186, 180, 181, 182 and 17 (according to IMGT);
3) SEQ ID NOs: 323, 332, 325, 326, 182 and 334 (according to Chothia); or
4) SEQ ID NOs: 463, 472, 465, 466, 474 and 334 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 635 and 637 (antibody 10).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 16, 3, 11, 12 and 18 (according to Kabat);
2) SEQ ID NOs: 178, 187, 180, 181, 182 and 18 (according to IMGT);
3) SEQ ID NOs: 323, 333, 325, 326, 182 and 335 (according to Chothia); or
4) SEQ ID NOs: 463, 473, 465, 466, 474 and 335 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 636 and 638 (antibody 11).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1078, 3, 11, 8 and 9 (according to Kabat);
2) SEQ ID NOs: 178, 185, 180, 181, 182 and 9 (according to IMGT);
3) SEQ ID NOs: 323, 331, 325, 326, 182 and 329 (according to Chothia); or
4) SEQ ID NOs: 463, 475, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 639 and 632 (antibody 12).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1079, 3, 11, 8 and 9 (according to Kabat);
2) SEQ ID NOs: 178, 188, 180, 181, 182 and 9 (according to IMGT);
3) SEQ ID NOs: 323, 336, 325, 326, 182 and 329 (according to Chothia); or
4) SEQ ID NOs: 463, 475, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 632 (antibody 13). In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 641 and 632 (antibody 14). In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 644 (antibody 17). In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 645 (antibody 18). In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 640 and 646 (antibody 19).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1080, 3, 11, 8 and 9 (according to Kabat);
2) SEQ ID NOs: 178, 189, 180, 181, 182 and 9 (according to IMGT);
3) SEQ ID NOs: 323, 337, 325, 326, 182 and 329 (according to Chothia); or
4) SEQ ID NOs: 463, 476, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 642 and 632 (antibody 15).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 1080, 3, 11, 8 and 9 (according to Kabat);
2) SEQ ID NOs: 178, 190, 180, 181, 182 and 9 (according to IMGT);
3) SEQ ID NOs: 323, 338, 325, 326, 182 and 329 (according to Chothia); or
4) SEQ ID NOs: 463, 477, 465, 466, 469 and 329 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 643 and 632 (antibody 16).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 20, 21, 22, 23 and 24 (according to Kabat);
2) SEQ ID NOs: 191, 192, 193, 194, 195 and 24 (according to IMGT);
3) SEQ ID NOs: 339, 340, 341, 342, 195 and 1131 (according to Chothia); or
4) SEQ ID NOs: 479, 480, 481, 482, 483 and 1131 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 647 and 648 (antibody 1.17).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 26, 27, 28, 29 and 30 (according to Kabat);
2) SEQ ID NOs: 196, 197, 198, 199, 200 and 30 (according to IMGT);
3) SEQ ID NOs: 344, 345, 346, 347, 200 and 348 (according to Chothia); or
4) SEQ ID NOs: 484, 485, 486, 487, 488 and 348 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 649 and 650 (antibody 1.25).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 31, 32, 33, 34, 29 and 35 (according to Kabat);
2) SEQ ID NOs: 201, 202, 203, 199, 200 and 35 (according to IMGT);
3) SEQ ID NOs: 349, 350, 351, 347, 200 and 352 (according to Chothia); or
4) SEQ ID NOs: 489, 490, 491, 487, 492 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 651 and 652 (antibody 1.26).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 36, 37, 28, 29 and 38 (according to Kabat);
2) SEQ ID NOs: 204, 205, 206, 199, 200 and 38 (according to IMGT);
3) SEQ ID NOs: 353, 354, 355, 347, 200 and 356 (according to Chothia); or
4) SEQ ID NOs: 493, 494, 495, 487, 492 and 356 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 653 and 654 (antibody 1.27).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 1082, 39, 28, 29 and 38 (according to Kabat);
2) SEQ ID NOs: 196, 207, 208, 199, 200 and 38 (according to IMGT);
3) SEQ ID NOs: 344, 350, 357, 347, 200 and 356 (according to Chothia); or
4) SEQ ID NOs: 484, 496, 497, 487, 492 and 356 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 655 and 656 (antibody 1.28).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 40, 41, 42, 28, 29 and 35 (according to Kabat);
2) SEQ ID NOs: 209, 210, 211, 199, 200 and 35 (according to IMGT);
3) SEQ ID NOs: 358, 350, 359, 347, 200 and 352 (according to Chothia); or
4) SEQ ID NOs: 498, 499, 500, 487, 492 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 657 and 658 (antibody 1.29).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 43, 39, 28, 29 and 38 (according to Kabat);
2) SEQ ID NOs: 196, 212, 208, 199, 200 and 38 (according to IMGT);
3) SEQ ID NOs: 343, 354, 357, 347, 200 and 356 (according to Chothia); or
4) SEQ ID NOs: 484, 501, 497, 487, 492 and 356 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 659 and 660 (antibody 1.30).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 44, 45, 28, 29 and 35 (according to Kabat);
2) SEQ ID NOs: 196, 213, 214, 199, 200 and 35 (according to IMGT);
3) SEQ ID NOs: 343, 360, 361, 347, 200 and 352 (according to Chothia); or
4) SEQ ID NOs: 484, 502, 503, 487, 492 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 661 and 662 (antibody 1.31).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 46, 47, 28, 29 and 35 (according to Kabat);
2) SEQ ID NOs: 196, 213, 215, 199, 200 and 35 (according to IMGT);
3) SEQ ID NOs: 343, 360, 362, 347, 200 and 352 (according to Chothia); or
4) SEQ ID NOs: 484, 504, 505, 487, 506 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 663 and 664 (antibody 1.32).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 48, 49, 28, 29 and 35 (according to Kabat);
2) SEQ ID NOs: 196, 216, 217, 199, 200 and 35 (according to IMGT);
3) SEQ ID NOs: 343, 363, 364, 347, 200 and 352 (according to Chothia); or
4) SEQ ID NOs: 484, 507, 508, 487, 492 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 665 and 666 (antibody 1.33).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 48, 50, 28, 29 and 35 (according to Kabat);
2) SEQ ID NOs: 196, 216, 218, 199, 200 and 35 (according to IMGT);
3) SEQ ID NOs: 343, 363, 365, 347, 200 and 352 (according to Chothia); or
4) SEQ ID NOs: 484, 507, 509, 487, 492 and 352 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 667 and 666 (antibody 1.34).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 51, 52, 53, 54, 55 and 56 (according to Kabat);
2) SEQ ID NOs: 219, 220, 221, 222, 223 and 56 (according to IMGT);
3) SEQ ID NOs: 366, 367, 368, 1084, 223 and 369 (according to Chothia); or
4) SEQ ID NOs: 510, 511, 512, 513, 514 and 369 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 668 and 669 (antibody 1.45).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 51, 57, 53, 58, 55 and 56 (according to Kabat);
2) SEQ ID NOs: 219, 224, 221, 225, 223 and 56 (according to IMGT);
3) SEQ ID NOs: 366, 367, 368, 370, 223 and 369 (according to Chothia); or
4) SEQ ID NOs: 510, 515, 512, 516, 514 and 369 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 670 and 671, 672 and 673, or 672 and 671 (antibody 1.46, 1.47 or 1.48, respectively).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 59, 60, 61, 62, 63 and 64 (according to Kabat);
2) SEQ ID NOs: 226, 227, 228, 229, 230 and 64 (according to IMGT);
3) SEQ ID NOs: 371, 372, 373, 374, 230 and 375 (according to Chothia); or
4) SEQ ID NOs: 517, 518, 519, 374, 520 and 375 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: (keliximab).

In some embodiments, the antigen binding domain that specifically binds to CD4 D1 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 65, 66, 67, 68, 69 and 70 (according to Kabat);
2) SEQ ID NOs: 231, 232, 233, 234, 235 and 70 (according to IMGT);
3) SEQ ID NOs: 376, 377, 378, 379, 235 and 380 (according to Chothia); or
4) SEQ ID NOs: 521, 522, 523, 524, 525 and 380 (according to Honegger).

In some embodiments, the antigen binding domain specifically binds to CD4 D1 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: (UB-421).

CD4 Binding Domains that Bind to CD4 D2

In some embodiments, the antigen binding domain that specifically binds to CD4 specifically binds to the CD4 D2 domain. An antigen binding domain that specifically binds to CD4 D2 is useful in combination therapies with molecules that comprise a CD4 D1 extracellular domain, e.g., a CD4 D1-Fc fusion protein, e.g., a molecule comprising CD4 D1.22, described in Chen, et al., J. Virol. (2014) 88(2):1125-1139. For example, an antigen binding domain that specifically binds to CD4 D2 is useful in combination therapies with molecules comprising CD4 D1, e.g., molecules comprising a CD4-Fc fusion protein described, e.g., in WO 2011/146891, WO 2019/183387, WO 2022/046644 (e.g., amtabafusp alfa (GS-8588)) and WO 2024/094690. In some embodiments, the antibodies or antigen-binding fragments thereof that specifically bind to CD4 D2, described herein, are insensitive to the amino acid substitutions resulting from CD4 polymorphism variant ID rs11064416 (F123L), wherein the amino acid positions are with reference to SEQ ID NO: 1120.

In some embodiments, the antigen binding domain specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of:
1) SEQ ID NOs: 71, 72, 73, 74, 75 and 76; (ibalizumab)
2) SEQ ID NOs: 77, 78, 79, 80, 81 and 82; (tregalizumab)
3) SEQ ID NOs: 25, 83, 84, 85, 86 and 87; (1.1)
4) SEQ ID NOs: 88, 89, 90, 91, 92 and 93; (1.4)
5) SEQ ID NOs: 94, 95, 96, 97, 98 and 99; (1.5)
6) SEQ ID NOs: 88, 100, 101, 102, 103 and 104; (2.9)
7) SEQ ID NOs: 88, 105, 106, 107, 108 and 93; (2.10)
8) SEQ ID NOs: 88, 109, 110, 111, 108 and 112; (2.11)
9) SEQ ID NOs: 88, 109, 110, 111, 113 and 112; (2.11 VL N50G)
10) SEQ ID NOs: 88, 114, 106, 107, 108 and 115; (2.12)
11) SEQ ID NOs: 88, 114, 106, 107, 113 and 115; (2.12 VL N50G)
12) SEQ ID NOs: 88, 116, 117, 118, 108 and 119; (2.13)
13) SEQ ID NOs: 120, 121, 122, 102, 113 and 123; (2.14) or
14) SEQ ID NOs: 88, 124, 122, 125, 113 and 104; (2.15).

In some embodiments, the antigen binding domain specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of:
1) SEQ ID NOs: 236, 237, 238, 239, 240 and 76; (ibalizumab)
2) SEQ ID NOs: 241, 242, 243, 244, 245 and 82; (tregalizumab)
3) SEQ ID NOs: 246, 247, 248, 249, 250 and 87; (1.1)
4) SEQ ID NOs: 251, 252, 253, 254, 255 and 93; (1.4)
5) SEQ ID NOs: 256, 257, 258, 259, 255 and 99; (1.5)
6) SEQ ID NOs: 251, 260, 261, 262, 263 and 104; (2.9)
7) SEQ ID NOs: 251, 264, 265, 266, 267 and 93; (2.10)
8) SEQ ID NOs: 251, 268, 269, 270, 267 and 112; (2.11)
9) SEQ ID NOs: 251, 268, 269, 270, 263 and 112; (2.11 VL N50G)
10) SEQ ID NOs: 251, 271, 265, 266, 267 and 115; (2.12)
11) SEQ ID NOs: 251, 271, 265, 266, 263 and 115; (2.12 VL N50G)
12) SEQ ID NOs: 251, 272, 273, 274, 267 and 119; (2.13)
13) SEQ ID NOs: 275, 276, 277, 262, 263 and 123; (2.14) or
14) SEQ ID NOs: 251, 278, 277, 279, 263 and 104; (2.15).

In some embodiments, the antigen binding domain that specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of:
1) SEQ ID NOs: 366, 381, 382, 383, 240 and 384; (ibalizumab)
2) SEQ ID NOs: 385, 386, 387, 388, 245 and 389; (tregalizumab)
3) SEQ ID NOs: 390, 391, 392, 393, 250 and 394; (1.1)
4) SEQ ID NOs: 395, 396, 397, 398, 255 and 399; (1.4)
5) SEQ ID NOs: 400, 401, 402, 403, 255 and 404; (1.5)
6) SEQ ID NOs: 395, 405, 406, 407, 263 and 408; (2.9)
7) SEQ ID NOs: 395, 409, 410, 411, 267 and 399; (2.10)
8) SEQ ID NOs: 395, 412, 413, 414, 267 and 399; (2.11)
9) SEQ ID NOs: 395, 412, 413, 414, 263 and 399; (2.11 VL N50G)
10) SEQ ID NOs: 395, 415, 410, 411, 267 and 408; (2.12)
11) SEQ ID NOs: 395, 415, 410, 411, 263 and 408; (2.12 VL N50G)
12) SEQ ID NOs: 395, 416, 417, 418, 267 and 419; (2.13)
13) SEQ ID NOs: 420, 421, 422, 407, 263 and 408; (2.14) or
14) SEQ ID NOs: 395, 423, 422, 424, 263 and 408; (2.15).

In some embodiments, the antigen binding domain that specifically binds to CD4 D2 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of:
1) SEQ ID NOs: 526, 527, 528, 529, 530 and 384; (ibalizumab)
2) SEQ ID NOs: 531, 532, 533, 534, 535 and 389; (tregalizumab)
3) SEQ ID NOs: 536, 537, 538, 539, 540 and 394; (1.1)
4) SEQ ID NOs: 541, 542, 543, 544, 545 and 399; (1.4)
5) SEQ ID NOs: 546, 547, 548, 549, 550 and 404; (1.5)
6) SEQ ID NOs: 541, 551, 552, 553, 554 and 408; (2.9)
7) SEQ ID NOs: 541, 555, 556, 557, 558 and 399; (2.10)
8) SEQ ID NOs: 541, 559, 560, 561, 562 and 399; (2.11)
9) SEQ ID NOs: 541, 559, 560, 561, 563 and 399; (2.11 VL N50G)
10) SEQ ID NOs: 541, 564, 556, 557, 558 and 408; (2.12)
11) SEQ ID NOs: 541, 564, 556, 557, 565 and 408; (2.12 VL N50G)
12) SEQ ID NOs: 541, 566, 567, 568, 569 and 419; (2.13)
13) SEQ ID NOs: 570, 571, 572, 553, 565 and 408; (2.14) or
14) SEQ ID NOs: 541, 573, 572, 574, 575 and 408; (2.15).

In some embodiments, the antigen binding domain that specifically binds to CD4 D2 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 678 and 679; (ibalizumab)
2) SEQ ID NOs: 680 and 681; (tregalizumab)
3) SEQ ID NOs: 682 and 683; (1.1)
4) SEQ ID NOs: 684 and 685; (1.4)
5) SEQ ID NOs: 686 and 687; (1.5)
6) SEQ ID NOs: 688 and 689; (2.9)
7) SEQ ID NOs: 690 and 691; (2.10)
8) SEQ ID NOs: 692 and 693; (2.11)
9) SEQ ID NOs: 692 and 694; (2.11 VL N50G)
10) SEQ ID NOs: 695 and 696; (2.12)
11) SEQ ID NOs: 695 and 697; (2.12 VL N50G)
12) SEQ ID NOs: 698 and 699; (2.13)
13) SEQ ID NOs: 700 and 701; (2.14) or
14) SEQ ID NOs: 702 and 703; (2.15).

In some embodiments, antigen binding domain that specifically binds to CD4 D2 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 71, 72, 73, 74, 75 and 76 (according to Kabat);
2) SEQ ID NOs: 236, 237, 238, 239, 240 and 76 (according to IMGT);
3) SEQ ID NOs: 366, 381, 382, 383, 240 and 384 (according to Chothia); or
4) SEQ ID NOs: 526, 527, 528, 529, 530 and 384 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 678 and 679 (ibalizumab).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 77, 78, 79, 80, 81 and 82 (according to Kabat);
2) SEQ ID NOs: 241, 242, 243, 244, 245 and 82 (according to IMGT);
3) SEQ ID NOs: 385, 386, 387, 388, 245 and 389 (according to Chothia); or
4) SEQ ID NOs: 531, 532, 533, 534, 535 and 389 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 680 and 681 (tregalizumab).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 83, 84, 85, 86 and 87 (according to Kabat);
2) SEQ ID NOs: 246, 247, 248, 249, 250 and 87 (according to IMGT);
3) SEQ ID NOs: 390, 391, 392, 393, 250 and 394 (according to Chothia); or
4) SEQ ID NOs: 536, 537, 538, 539, 540 and 394 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 682 and 683 (antibody SCT1.1).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 89, 90, 91, 92 and 93 (according to Kabat);
2) SEQ ID NOs: 251, 252, 253, 254, 255 and 93 (according to IMGT);
3) SEQ ID NOs: 395, 396, 397, 398, 255 and 399 (according to Chothia); or
4) SEQ ID NOs: 541, 542, 543, 544, 545 and 399 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 684 and 685 (antibody SCT1.4).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 94, 95, 96, 97, 98 and 99 (according to Kabat);
2) SEQ ID NOs: 256, 257, 258, 259, 255 and 99 (according to IMGT);
3) SEQ ID NOs: 400, 401, 402, 403, 255 and 404 (according to Chothia); or
4) SEQ ID NOs: 546, 547, 548, 549, 550 and 404 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 686 and 687 (antibody SCT1.5).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 100, 101, 102, 103 and 104 (according to Kabat);
2) SEQ ID NOs: 251, 260, 261, 262, 263 and 104 (according to IMGT);
3) SEQ ID NOs: 395, 405, 406, 407, 263 and 408 (according to Chothia); or
4) SEQ ID NOs: 541, 551, 552, 553, 554 and 408 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 688 and 689 (antibody SCT2.9).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 105, 106, 107, 108 and 93 (according to Kabat);
2) SEQ ID NOs: 251, 264, 265, 266, 267 and 93 (according to IMGT);
3) SEQ ID NOs: 395, 409, 410, 411, 267 and 399 (according to Chothia); or
4) SEQ ID NOs: 541, 555, 556, 557, 558 and 399 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 690 and 691 (antibody SCT2.10).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 109, 110, 111, 108 and 112 (according to Kabat);
2) SEQ ID NOs: 251, 268, 269, 270, 267 and 112 (according to IMGT);
3) SEQ ID NOs: 395, 412, 413, 414, 267 and 399 (according to Chothia); or
4) SEQ ID NOs: 541, 559, 560, 561, 562 and 399 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 692 and 693 (antibody SCT2.11).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 109, 110, 111, 113 and 112 (according to Kabat);
2) SEQ ID NOs: 251, 268, 269, 270, 263 and 112 (according to IMGT);
3) SEQ ID NOs: 395, 412, 413, 414, 263 and 399 (according to Chothia); or
4) SEQ ID NOs: 541, 559, 560, 561, 563 and 399 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 692 and 694 (antibody SCT2.11 VL N50G).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 114, 106, 107, 108 and 115 (according to Kabat);
2) SEQ ID NOs: 251, 271, 265, 266, 267 and 115 (according to IMGT);
3) SEQ ID NOs: 395, 415, 410, 411, 267 and 408 (according to Chothia); or
4) SEQ ID NOs: 541, 564, 556, 557, 558 and 408 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 695 and 696 (antibody SCT2.12).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 114, 106, 107, 113 and 115 (according to Kabat);
2) SEQ ID NOs: 251, 271, 265, 266, 263 and 115 (according to IMGT);
3) SEQ ID NOs: 395, 415, 410, 411, 263 and 408 (according to Chothia); or
4) SEQ ID NOs: 541, 564, 556, 557, 565 and 408 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 695 and 697 (antibody SCT2.12 VL N50G).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 116, 117, 118, 108 and 119 (according to Kabat);
2) SEQ ID NOs: 251, 272, 273, 274, 267 and 119 (according to IMGT);
3) SEQ ID NOs: 395, 416, 417, 418, 267 and 419 (according to Chothia); or
4) SEQ ID NOs: 541, 566, 567, 568, 569 and 419 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 698 and 699 (antibody SCT2.13).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 120, 121, 122, 102, 113 and 123 (according to Kabat);
2) SEQ ID NOs: 275, 276, 277, 262, 263 and 123 (according to IMGT);
3) SEQ ID NOs: 420, 421, 422, 407, 263 and 408 (according to Chothia); or
4) SEQ ID NOs: 570, 571, 572, 553, 565 and 408 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 700 and 701 (antibody SCT2.14).

In some embodiments, provided is an antibody or antigen-binding fragment thereof that specifically binds to CD4 D2 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 88, 124, 122, 125, 113 and 104 (according to Kabat);
2) SEQ ID NOs: 251, 278, 277, 279, 263 and 104 (according to IMGT);
3) SEQ ID NOs: 395, 423, 422, 424, 263 and 408 (according to Chothia); or
4) SEQ ID NOs: 541, 573, 572, 574, 575 and 408 (according to Honegger).

In some embodiments, the antigen binding domain that specifically binds to CD4 D2, wherein the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 702 and 703 (antibody SCT2.15).

CD4 Binding Domains that Bind to One or Both of CD4 D2 and D3

In some embodiments, the antigen binding domain that specifically binds to CD4 specifically binds to one or both of the CD4 D2 domain and the CD4 D3 domain. An antigen binding domain that specifically bind to one or both of CD4 D2 and CD4 D3 are useful in combination therapies with molecules that comprise a CD4 D1 extracellular domain, e.g., a CD4 D1-Fc fusion protein, e.g., a molecule comprising CD4 D1.22, described in Chen, et al., J. Virol. (2014) 88(2):1125-1139. For example, an antigen binding domain that specifically bind to one or both of CD4 D2 and CD4 D3 are useful in combination therapies with molecules comprising CD4 D1, e.g., molecules comprising a CD4-Fc fusion protein described, e.g., in WO 2011/146891, WO 2019/183387, WO 2022/046644 (e.g., amtabafusp alfa (GS-8588)) and WO 2024/094690. In some embodiments, the antibodies or antigen-binding fragments thereof that specifically bind to one or both of CD4 D2 and CD4 D3, described herein, are insensitive to the amino acid substitutions resulting from one or more of CD4 polymorphism variant IDs rs28919570 (R265W), rs11064419 (F227S or F227C) and rs11064416 (F123L), wherein the amino acid positions are with reference to SEQ ID NO: 1120.

In some embodiments, the antigen binding domain specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of:
1) SEQ ID NOs: 126, 127, 128, 129, 130 and 99; (1.6)
2) SEQ ID NOs: 126, 131, 132, 129, 133 and 99; (1.7)
3) SEQ ID NOs: 126, 134, 135, 136, 133 and 99; (1.8)
4) SEQ ID NOs: 126, 137, 128, 129, 138 and 99; (1.9)
5) SEQ ID NOs: 126, 139, 128, 140, 141 and 99; (1.10)
6) SEQ ID NOs: 142, 143, 128, 145, 141 and 99; (1.19)
7) SEQ ID NOs: 126, 146, 147, 140, 141 and 99; (2.2) or
8) SEQ ID NOs: 148, 149, 150, 129, 151 and 99; (2.3).

In some embodiments, the antigen binding domain specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of:
1) SEQ ID NOs: 280, 281, 282, 283, 284 and 99; (1.6)
2) SEQ ID NOs: 280, 285, 286, 283, 284 and 99; (1.7)
3) SEQ ID NOs: 280, 287, 288, 283, 284 and 99; (1.8)
4) SEQ ID NOs: 289, 290, 282, 283, 284 and 99; (1.9)
5) SEQ ID NOs: 289, 291, 292, 293, 255 and 99; (1.10)
6) SEQ ID NOs: 294, 295, 296, 297, 255 and 99; (1.19)
7) SEQ ID NOs: 289, 298, 299, 293, 255 and 99; (2.2) or
8) SEQ ID NOs: 289, 300, 301, 283, 284 and 99; (2.3).

In some embodiments, the antigen binding domain that specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of:
1) SEQ ID NOs: 425, 426, 427, 428, 284 and 404; (1.6)
2) SEQ ID NOs: 425, 426, 429, 428, 284 and 404; (1.7)
3) SEQ ID NOs: 425, 426, 430, 428, 284 and 404; (1.8)
4) SEQ ID NOs: 431, 432, 427, 428, 284 and 404; (1.9)
5) SEQ ID NOs: 431, 433, 427, 434, 255 and 404; (1.10)
6) SEQ ID NOs: 431, 435, 436, 437, 255 and 404; (1.19)
7) SEQ ID NOs: 431, 438, 439, 434, 255 and 404; (2.2) or
8) SEQ ID NOs: 431, 438, 440, 428, 284 and 404; (2.3).

In some embodiments, the antigen binding domain that specifically binds to one or both of CD4 D2 and CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of:
1) SEQ ID NOs: 576, 577, 578, 579, 580 and 404; (1.6)
2) SEQ ID NOs: 576, 581, 582, 579, 583 and 404; (1.7)
3) SEQ ID NOs: 576, 584, 585, 586, 583 and 404; (1.8)
4) SEQ ID NOs: 587, 588, 578, 579, 589 and 404; (1.9)
5) SEQ ID NOs: 587, 590, 578, 591, 592 and 404; (1.10)
6) SEQ ID NOs: 593, 594, 595, 596, 597 and 404; (1.19)
7) SEQ ID NOs: 587, 598, 599, 591, 597 and 404; (2.2) or
8) SEQ ID NOs: 587, 600, 601, 579, 602 and 404; (2.3).

In some embodiments, the antigen binding domain that specifically binds to one or both of CD4 D2 and CD4 D3 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 704 and 705; (1.6)
2) SEQ ID NOs: 706 and 707; (1.7)
3) SEQ ID NOs: 708 and 709; (1.8)
4) SEQ ID NOs: 710 and 711; (1.9)
5) SEQ ID NOs: 712 and 713; (1.10)
6) SEQ ID NOs: 714 and 715; (1.19)
7) SEQ ID NOs: 716 and 717; (2.2) or
8) SEQ ID NOs: 718 and 719; (2.3).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 127, 128, 129, 130 and 99 (according to Kabat);
2) SEQ ID NOs: 280, 281, 282, 283, 284 and 99 (according to IMGT);
3) SEQ ID NOs: 425, 426, 427, 428, 284 and 404 (according to Chothia); or
4) SEQ ID NOs: 576, 577, 578, 579, 580 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 704 and 705 (antibody SCT1.6).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 131, 132, 129, 133 and 99 (according to Kabat);
2) SEQ ID NOs: 280, 285, 286, 283, 284 and 99 (according to IMGT);
3) SEQ ID NOs: 425, 426, 429, 428, 284 and 404 (according to Chothia); or
4) SEQ ID NOs: 576, 581, 582, 579, 583 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 706 and 707 (antibody SCT1.7).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 134, 135, 136, 133 and 99 (according to Kabat);
2) SEQ ID NOs: 280, 287, 288, 283, 284 and 99 (according to IMGT);
3) SEQ ID NOs: 425, 426, 429, 428, 284 and 404 (according to Chothia); or
4) SEQ ID NOs: 576, 584, 585, 586, 583 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 708 and 709 (antibody SCT1.8).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 137, 128, 129, 138 and 99 (according to Kabat);
2) SEQ ID NOs: 289, 290, 282, 283, 284 and 99 (according to IMGT);
3) SEQ ID NOs: 431, 432, 427, 428, 284 and 404 (according to Chothia); or
4) SEQ ID NOs: 587, 588, 578, 579, 589 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 710 and 711 (antibody SCT1.9).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 139, 128, 140, 141 and 99 (according to Kabat);
2) SEQ ID NOs: 289, 291, 292, 293, 255 and 99 (according to IMGT);
3) SEQ ID NOs: 431, 433, 427, 434, 255 and 404 (according to Chothia); or
4) SEQ ID NOs: 587, 590, 578, 591, 592 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 712 and 713 (antibody SCT1.10).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 142, 143, 128, 145, 141 and 99 (according to Kabat);
2) SEQ ID NOs: 294, 295, 296, 297, 255 and 99 (according to IMGT);
3) SEQ ID NOs: 431, 435, 436, 437, 255 and 404 (according to Chothia); or
4) SEQ ID NOs: 593, 594, 595, 596, 597 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 714 and 715 (antibody SCT1.19).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 126, 146, 147, 140, 141 and 99 (according to Kabat);
2) SEQ ID NOs: 289, 298, 299, 293, 255 and 99 (according to IMGT);
3) SEQ ID NOs: 431, 438, 439, 434, 255 and 404 (according to Chothia); or
4) SEQ ID NOs: 587, 598, 599, 591, 597 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 716 and 717 (antibody SCT2.2).

In some embodiments, the antigen binding domain that specifically binds to one or both of CD4 D2 and CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 148, 149, 150, 129, 151 and 99 (according to Kabat);
2) SEQ ID NOs: 289, 300, 301, 283, 284 and 99 (according to IMGT);
3) SEQ ID NOs: 431, 438, 440, 428, 284 and 404 (according to Chothia); or
4) SEQ ID NOs: 587, 600, 601, 579, 602 and 404 (according to Honegger).

In some embodiments, the antigen binding domain specifically binds to one or both of CD4 D2 and CD4 D3 and the VH and VL comprise, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 718 and 719 (antibody SCT2.3).

### CD4 Binding Domains That Bind to CD4 D3

In some embodiments, the antigen binding domain that specifically binds to CD4 specifically binds to the CD4 D3 domain. An antigen binding domain that specifically bind to CD4 D3 are useful in combination therapies with molecules that comprise a CD4 D1 or a CD4 D1D2 extracellular domain, e.g., a CD4 D1-Fc fusion protein, e.g., a molecule comprising CD4 D1.22 (with or without a D2), described in Chen, et al., J. Virol. (2014) 88(2):1125-1139. For example, an antigen binding domain that specifically bind to CD4 D3 are useful in combination therapies with molecules comprising CD4 D1 or CD4 D1D2, e.g., molecules comprising a CD4-Fc fusion protein described, e.g., in U.S. Pat. No. 7,368,114; and Intl. Publ. Nos. WO 2011/146891, WO 2019/183387, WO 2022/046644 (e.g., amtabafusp alfa (GS-8588)) and WO 2024/094690. In some embodiments, the antibodies or antigen-binding fragments thereof that specifically bind to CD4 D3, described herein, are insensitive to the amino acid substitutions resulting from one or more of CD4 polymorphism variant IDs rs28919570 (R265W) and rs11064419 (F227S or F227C), wherein the amino acid positions are with reference to SEQ ID NO: 1120. In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120. In some embodiments, the antibodies or antigen-binding fragments thereof that specifically bind to CD4 D3 bind to CD4 D3 with a binding equilibrium dissociation constant (K_{D}) of lower than 1 nM, e.g., lower than 100 pM, lower than 90 pM, lower than 80 pM, lower than 70 pM, lower than 60 pM, lower than 50 pM, lower than 40 pM, or lower than 30 pM.

In some embodiments, the antigen binding domain specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of:
1) SEQ ID NOs: 152, 153, 154, 155, 156 and 157; (OKT4)
2) SEQ ID NOs: 158, 159, 160, 145, 161 and 162; (1.20)
3) SEQ ID NOs: 163, 164, 160, 165, 166 and 162; (1.21)
4) SEQ ID NOs: 163, 164, 160, 165, 161 and 162; (1.22)
5) SEQ ID NOs: 158, 167, 168, 145, 98 and 169; (1.23)
6) SEQ ID NOs: 170, 171, 172, 173, 141 and 99; (2.1) or
7) SEQ ID NOs: 25, 174, 175, 176, 29 and 177; (2.8).

In some embodiments, the antigen binding domain specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to IMGT) of:
1) SEQ ID NOs: 302, 303, 304, 305, 245 and 157; (OKT4)
2) SEQ ID NOs: 306, 307, 1083, 297, 308 and 162; (1.20)
3) SEQ ID NOs: 309, 310, 311, 312, 313 and 162; (1.21)
4) SEQ ID NOs: 309, 310, 311, 312, 308 and 162; (1.22)
5) SEQ ID NOs: 314, 315, 316, 297, 255 and 169; (1.23)
6) SEQ ID NOs: 317, 318, 319, 320, 255 and 99; (2.1) or
7) SEQ ID NOs: 196, 321, 322, 199, 200 and 177; (2.8).

In some embodiments, the antigen binding domain that specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Chothia) of:
1) SEQ ID NOs: 441, 442, 443, 444, 245 and 445; (OKT4)
2) SEQ ID NOs: 446, 447, 448, 437, 308 and 449; (1.20)
3) SEQ ID NOs: 450, 451, 448, 452, 313 and 449; (1.21)
4) SEQ ID NOs: 450, 451, 448, 452, 308 and 449; (1.22)
5) SEQ ID NOs: 453, 454, 455, 437, 255 and 456; (1.23)
6) SEQ ID NOs: 457, 458, 459, 460, 255 and 404; (2.1) or
7) SEQ ID NOs: 343, 350, 461, 347, 200 and 462; (2.8).

In some embodiments, the antigen binding domain that specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Honegger) of:
1) SEQ ID NOs: 603, 604, 605, 606, 607 and 445; (OKT4)
2) SEQ ID NOs: 608, 609, 610, 596, 611 and 449; (1.20)
3) SEQ ID NOs: 612, 613, 610, 614, 615 and 449; (1.21)
4) SEQ ID NOs: 612, 613, 610, 614, 611 and 449; (1.22)
5) SEQ ID NOs: 616, 617, 618, 596, 550 and 456; (1.23)
6) SEQ ID NOs: 619, 620, 621, 622, 597 and 404; (2.1) or
7) SEQ ID NOs: 484, 623, 624, 487, 492 and 462; (2.8).

In some embodiments, the antigen binding domain that specifically binds to CD4 D3 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 720 and 721; (OKT4)
2) SEQ ID NOs: 722 and 723; (1.20)
3) SEQ ID NOs: 724 and 725; (1.21)
4) SEQ ID NOs: 724 and 726; (1.22)
5) SEQ ID NOs: 727 and 728; (1.23)
6) SEQ ID NOs: 727 and 729; (1.23 VL C36Y)
7) SEQ ID NOs: 730 and 731; (2.1) or
8) SEQ ID NOs: 732 and 733; (2.8).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 158, 159, 160, 145, 161 and 162 (according to Kabat);
2) SEQ ID NOs: 306, 307, 1083, 297, 308 and 162 (according to IMGT);
3) SEQ ID NOs: 446, 447, 448, 437, 308 and 449 (according to Chothia); or
4) SEQ ID NOs: 608, 609, 610, 596, 611 and 449 (according to Honegger).

In some embodiments, the antigen binding domain specifically binds to CD4 D3 and the VH and VL comprise the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 722 and 723 (antibody SCT1.20).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
5) SEQ ID NOs: 163, 164, 160, 165, 166 and 162 (according to Kabat);
6) SEQ ID NOs: 309, 310, 311, 312, 313 and 162 (according to IMGT);
7) SEQ ID NOs: 450, 451, 448, 452, 313 and 449 (according to Chothia); or
8) SEQ ID NOs: 612, 613, 610, 614, 615 and 449 (according to Honegger).

In some embodiments, the antigen binding domain that specifically binds to CD4 D3 comprises

VH and VL, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 724 and 725 (antibody SCT1.21).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 163, 164, 160, 165, 161 and 162 (according to Kabat);
2) SEQ ID NOs: 309, 310, 311, 312, 308 and 162 (according to IMGT);
3) SEQ ID NOs: 450, 451, 448, 452, 308 and 449 (according to Chothia); or
4) SEQ ID NOs: 612, 613, 610, 614, 611 and 449 (according to Honegger).

In some embodiments, provided is an antibody or antigen-binding fragment that specifically binds to CD4 D3, wherein the VH and VL comprise, respectively, comprising the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 724 and 726. In some embodiments, the antibody or antigen-binding fragment thereof binds to an epitope within CD4 D3 comprising amino acid residues at positions 218, 220, 260, 271, 274-277, 279, 283 and 285, wherein the residue positions are with reference to SEQ ID NO: 1120 (antibody SCT1.22).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 158, 167, 168, 145, 98 and 169 (according to Kabat);
2) SEQ ID NOs: 314, 315, 316, 297, 255 and 169 (according to IMGT);
3) SEQ ID NOs: 453, 454, 455, 437, 255 and 456 (according to Chothia); or
4) SEQ ID NOs: 616, 617, 618, 596, 550 and 456 (according to Honegger).

In some embodiments, the antigen binding domain specifically binds to CD4 D3 and the VH and VL comprise the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 727 and 728 or 727 and 729 (antibody SCT1.23 and SCT1.23 VL C36Y).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 170, 171, 172, 173, 141 and 99 (according to Kabat);
2) SEQ ID NOs: 317, 318, 319, 320, 255 and 99 (according to IMGT);
3) SEQ ID NOs: 457, 458, 459, 460, 255 and 404 (according to Chothia); or
4) SEQ ID NOs: 619, 620, 621, 622, 597 and 404 (according to Honegger).

In some embodiments, the antigen binding domain specifically binds to CD4 D3 and the VH and VL comprise the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 730 and 731 (antibody SCT2.1).

In some embodiments, the antigen binding domain that specifically binds to CD4 D3 comprises a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 25, 174, 175, 176, 29 and 177 (according to Kabat);
2) SEQ ID NOs: 196, 321, 322, 199, 200 and 177 (according to IMGT);
3) SEQ ID NOs: 343, 350, 461, 347, 200 and 462 (according to Chothia); or
4) SEQ ID NOs: 484, 623, 624, 487, 492 and 462 (according to Honegger).

In some embodiments, the antigen binding domain specifically binds to CD4 D3 and the VH and VL comprise the amino acid sequences set forth below, or amino acid sequences at least 90%, 91%, 92%, 93%, 94%, 95%, 96%, 97%, 98%, 99% identical to, respectively, the full length of the amino acid sequences set forth in SEQ ID NOs: 732 and 733 (antibody SCT2.8).

The amino acid sequences of illustrative anti-CD4 antigen binding domains, CDRs and VH/VL, are provided in Tables A1-A4 and Table B, supra.

c. IL-15RA SUSHI Domain-IL-15v Fusion Polypeptide

The herein described CD4-targeted IL-15v molecules have a fusion protein comprising an IL-15RA SUSHI domain, an IL-15v fusion protein and an Fc. In some embodiments, the IL-15v has attenuated binding to IL-2βγ (CD122, NCBI Gene ID: 3560; CD132, NCBI Gene ID: 3561), e.g., in comparison to wild-type IL-15. In some embodiments, the IL-15v binds to IL-2Rβ with a binding equilibrium dissociation constant (K_{D}) of at least 1 μM, e.g., a K_{D} of at least 1.5 μM, at least 2 μM, at least 2.5 μM, at least 3 μM, at least 3.5 μM, at least 4 μM, at least 4.5 μM or at least 5 μM. In some embodiments, the IL-15v binds to IL-2Rβγ with a binding equilibrium dissociation constant (K_{D}) of at least 1 μM, e.g., a K_{D} of at least 3 nM, e.g., at least 4 nM, at least 5 nM, at least 6 nM, at least 7 nM, at least 8 nM, at least 9 nM, at least 10 nM, at least 11 nM, at least 12 nM, at least 13 nM, at least 14 nM, or at least 15 nM. In some embodiments, the IL15RA SUSHI domain-IL-15v fusion proteins described herein induce CD4+ T cell proliferation with an EC₅₀ of less than 5 nM, e.g., less than 4 nM, less than 3 nM, less than 2 nM, less than 1 nM, e.g., wherein CD4+ T cell proliferation potency is measured by marker of proliferation Ki-67 activation (MKI67; NCBI Gene ID: 4288). In some embodiments, the IL15RA SUSHI domain-IL-15v fusion protein induces CD8+ T cells and/or natural killer (NK) cell proliferation with an EC50 of greater than 100 nM, e.g., wherein CD8+ T cell and/or NK cell proliferation potency is measured by Ki-67 activation. In some embodiments, the IL15RA SUSHI domain-IL-15v fusion protein described herein induce CD4+ T cell proliferation with a potency that is at least 100-fold, e.g., at least 200-fold, at least 300-fold, at least 400-fold, at least 500-fold, at least 600-fold, or more, in comparison to the potency for inducing CD8+ T cell or NK cell proliferation, e.g., wherein cell proliferation potency is measured by Ki-67 activation.

### IL-15v

In some embodiments the CD4-targeted IL-15v molecule comprises an IL-15v comprising one or more of the following amino acid substitutions: N1D, N4D, S7G, D8N, D30N, D61N, E64Q, N65A, N65D, I68A, N71L, N71E, N72D, N77L, N79G, N79P, Q108E, N112A, N112G, N112D, N112S or N112K; or one of the following combinations of amino acid substitutions: N1D, N4D and D8N; N1D and D61N; N1D and E64Q; N1D and N65D; N4D and D61N; N4D and E64Q; N4D and N65D; S7G and N65D; D8G and D61N; D8G and E64Q; D8G and N65D; D8N and D61N; D8N and E64Q D8N and N65D; D30N and N65D; D61N and E64Q; D61N and N65D; E64Q and N65D; E64Q and Q108E; N65A and 168A; N65D and I68A; N71L and N79P; N71L and N112D; N79P and N112D; S7G, N65A and 168A; S7G, N65D and 168A; D30N, E64Q and N65D; D61N, E64Q and N65D; D61N, N65D and 168A; N71Q, N79Q and N112Q; N71A, N79A and N112A; N71G, N79G and N112G; N71S, N79L and N112E; N71E, N79P and N112K; N71L, N79P and N112K; N71L, N79P and N112D; N71K, N79P and N112D; N71E, N79P and N112S; N71E, N79P and N112D; N1D, D61N, E64Q and Q108E; N4D, D61N, E64Q and Q108E; N71Q, N77L, N79Q and N112Q; N71A, N77L, N79A and N112A; N71G, N77L, N79G and N112G; N71S, N77L, N79L and N112E; N71E, N77L, N79P and N112K; N71L, N77L, N79P and N112K; N71E, N77L, N79P and N112S; N71E, N77L, N79P and N112D; S7G, N65D, N71L, N79P and N112D; S7G, N65D, N71E, N77L, N79P and N112D; S7G, N65D, N71E, N77L, N79P and N112S; S7G, N65D, N71L, N77L, N79P and N112K; or S7G, N65D, N71E, N77L, N79P and N112K; wherein the position numbers are with respect to SEQ ID NO: 740 (wild-type IL-15).

In some embodiments, the CD4-targeted IL-15v molecule comprises an IL-15v as provided in Table D, e.g., having an amino acid sequence selected from SEQ ID NOs: 741-813, 1124 and 1125. In some embodiments, the CD4-targeted IL-15v molecule comprises an IL-15v comprising an amino acid sequence of any one of SEQ ID NOs: 743, 750-752, 754-756, 758, 768-771, 774, 777, 779, 783-788, 790-798, 801-808, 810-813 and 1125. In some embodiments, the CD4-targeted IL-15v molecule comprises an IL-15v comprising an amino acid sequence of SEQ ID NO: 743. In some embodiments, the CD4-targeted IL-15v molecule comprises an IL-15v comprising an amino acid sequence of SEQ ID NO: 744. In some embodiments, the CD4-targeted IL-15v molecule comprises an IL-15v comprising an amino acid sequence of SEQ ID NO: 745. In some embodiments, the CD4-targeted IL-15v molecule comprises an IL-15v comprising an amino acid sequence of SEQ ID NO: 746. In some embodiments, the CD4-targeted IL-15v molecule comprises an IL-15v comprising an amino acid sequence of SEQ ID NO: 747. In some embodiments, the CD4-targeted IL-15v molecule comprises an IL-15v comprising an amino acid sequence of SEQ ID NO: 748. In some embodiments, the CD4-targeted IL-15v molecule comprises an IL-15v comprising an amino acid sequence of SEQ ID NO: 749. In some embodiments, the CD4-targeted IL-15v molecule comprises an IL-15v comprising an amino acid sequence of SEQ ID NO: 750. In some embodiments, the CD4-targeted IL-15v molecule comprises an IL-15v comprising an amino acid sequence of SEQ ID NO: 751. In some embodiments, the CD4-targeted IL-15v molecule comprises an IL-15v comprising an amino acid sequence of SEQ ID NO: 752. In some embodiments, the CD4-targeted IL-15v molecule comprises an IL-15v comprising an amino acid sequence of SEQ ID NO: 753. In some embodiments, the CD4-targeted IL-15v molecule comprises an IL-15v comprising an amino acid sequence of SEQ ID NO: 754. In some embodiments, the CD4-targeted IL-15v molecule comprises an IL-15v comprising an amino acid sequence of SEQ ID NO: 755. In some embodiments, the CD4-targeted IL-15v molecule comprises an IL-15v comprising an amino acid sequence of SEQ ID NO: 756. In some embodiments, the CD4-targeted IL-15v molecule comprises an IL-15v comprising an amino acid sequence of SEQ ID NO: 757. In some embodiments, the CD4-targeted IL-15v molecule comprises an IL-15v comprising an amino acid sequence of SEQ ID NO: 758. In some embodiments, the CD4-targeted IL-15v molecule comprises an IL-15v comprising an amino acid sequence of SEQ ID NO: 759. In some embodiments, the CD4-targeted IL-15v molecule comprises an IL-15v comprising an amino acid sequence of SEQ ID NO: 760. In some embodiments, the CD4-targeted IL-15v molecule comprises an IL-15v comprising an amino acid sequence of SEQ ID NO: 761. In some embodiments, the CD4-targeted IL-15v molecule comprises an IL-15v comprising an amino acid sequence of SEQ ID NO: 762. In some embodiments, the CD4-targeted IL-15v molecule comprises an IL-15v comprising an amino acid sequence of SEQ ID NO: 763. In some embodiments, the CD4-targeted IL-15v molecule comprises an IL-15v comprising an amino acid sequence of SEQ ID NO: 764. In some embodiments, the CD4-targeted IL-15v molecule comprises an IL-15v comprising an amino acid sequence of SEQ ID NO: 765. In some embodiments, the CD4-targeted IL-15v molecule comprises an IL-15v comprising an amino acid sequence of SEQ ID NO: 766. In some embodiments, the CD4-targeted IL-15v molecule comprises an IL-15v comprising an amino acid sequence of SEQ ID NO: 767. In some embodiments, the CD4-targeted IL-15v molecule comprises an IL-15v comprising an amino acid sequence of SEQ ID NO: 768. In some embodiments, the CD4-targeted IL-15v molecule comprises an IL-15v comprising an amino acid sequence of SEQ ID NO: 769. In some embodiments, the CD4-targeted IL-15v molecule comprises an IL-15v comprising an amino acid sequence of SEQ ID NO: 770. In some embodiments, the CD4-targeted IL-15v molecule comprises an IL-15v comprising an amino acid sequence of SEQ ID NO: 771. In some embodiments, the CD4-targeted IL-15v molecule comprises an IL-15v comprising an amino acid sequence of SEQ ID NO: 772. In some embodiments, the CD4-targeted IL-15v molecule comprises an IL-15v comprising an amino acid sequence of SEQ ID NO: 773. In some embodiments, the CD4-targeted IL-15v molecule comprises an IL-15v comprising an amino acid sequence of SEQ ID NO: 774. In some embodiments, the CD4-targeted IL-15v molecule comprises an IL-15v comprising an amino acid sequence of SEQ ID NO: 775. In some embodiments, the CD4-targeted IL-15v molecule comprises an IL-15v comprising an amino acid sequence of SEQ ID NO: 776. In some embodiments, the CD4-targeted IL-15v molecule comprises an IL-15v comprising an amino acid sequence of SEQ ID NO: 777. In some embodiments, the CD4-targeted IL-15v molecule comprises an IL-15v comprising an amino acid sequence of SEQ ID NO: 778. In some embodiments, the CD4-targeted IL-15v molecule comprises an IL-15v comprising an amino acid sequence of SEQ ID NO: 779. In some embodiments, the CD4-targeted IL-15v molecule comprises an IL-15v comprising an amino acid sequence of SEQ ID NO: 780. In some embodiments, the CD4-targeted IL-15v molecule comprises an IL-15v comprising an amino acid sequence of SEQ ID NO: 781. In some embodiments, the CD4-targeted IL-15v molecule comprises an IL-15v comprising an amino acid sequence of SEQ ID NO: 782. In some embodiments, the CD4-targeted IL-15v molecule comprises an IL-15v comprising an amino acid sequence of SEQ ID NO: 783. In some embodiments, the CD4-targeted IL-15v molecule comprises an IL-15v comprising an amino acid sequence of SEQ ID NO: 784. In some embodiments, the CD4-targeted IL-15v molecule comprises an IL-15v comprising an amino acid sequence of SEQ ID NO: 785. In some embodiments, the CD4-targeted IL-15v molecule comprises an IL-15v comprising an amino acid sequence of SEQ ID NO: 786. In some embodiments, the CD4-targeted IL-15v molecule comprises an IL-15v comprising an amino acid sequence of SEQ ID NO: 787. In some embodiments, the CD4-targeted IL-15v molecule comprises an IL-15v comprising an amino acid sequence of SEQ ID NO: 788. In some embodiments, the CD4-targeted IL-15v molecule comprises an IL-15v comprising an amino acid sequence of SEQ ID NO: 789. In some embodiments, the CD4-targeted IL-15v molecule comprises an IL-15v comprising an amino acid sequence of SEQ ID NO: 790. In some embodiments, the CD4-targeted IL-15v molecule comprises an IL-15v comprising an amino acid sequence of SEQ ID NO: 791. In some embodiments, the CD4-targeted IL-15v molecule comprises an IL-15v comprising an amino acid sequence of SEQ ID NO: 792. In some embodiments, the CD4-targeted IL-15v molecule comprises an IL-15v comprising an amino acid sequence of SEQ ID NO: 793. In some embodiments, the CD4-targeted IL-15v molecule comprises an IL-15v comprising an amino acid sequence of SEQ ID NO: 794. In some embodiments, the CD4-targeted IL-15v molecule comprises an IL-15v comprising an amino acid sequence of SEQ ID NO: 795. In some embodiments, the CD4-targeted IL-15v molecule comprises an IL-15v comprising an amino acid sequence of SEQ ID NO: 796. In some embodiments, the CD4-targeted IL-15v molecule comprises an IL-15v comprising an amino acid sequence of SEQ ID NO: 797. In some embodiments, the CD4-targeted IL-15v molecule comprises an IL-15v comprising an amino acid sequence of SEQ ID NO: 798. In some embodiments, the CD4-targeted IL-15v molecule comprises an IL-15v comprising an amino acid sequence of SEQ ID NO: 799. In some embodiments, the CD4-targeted IL-15v molecule comprises an IL-15v comprising an amino acid sequence of SEQ ID NO: 800. In some embodiments, the CD4-targeted IL-15v molecule comprises an IL-15v comprising an amino acid sequence of SEQ ID NO: 801. In some embodiments, the CD4-targeted IL-15v molecule comprises an IL-15v comprising an amino acid sequence of SEQ ID NO: 802. In some embodiments, the CD4-targeted IL-15v molecule comprises an IL-15v comprising an amino acid sequence of SEQ ID NO: 803. In some embodiments, the CD4-targeted IL-15v molecule comprises an IL-15v comprising an amino acid sequence of SEQ ID NO: 804. In some embodiments, the CD4-targeted IL-15v molecule comprises an IL-15v comprising an amino acid sequence of SEQ ID NO: 805. In some embodiments, the CD4-targeted IL-15v molecule comprises an IL-15v comprising an amino acid sequence of SEQ ID NO: 806. In some embodiments, the CD4-targeted IL-15v molecule comprises an IL-15v comprising an amino acid sequence of SEQ ID NO: 807. In some embodiments, the CD4-targeted IL-15v molecule comprises an IL-15v comprising an amino acid sequence of SEQ ID NO: 808. In some embodiments, the CD4-targeted IL-15v molecule comprises an IL-15v comprising an amino acid sequence of SEQ ID NO: 809. In some embodiments, the CD4-targeted IL-15v molecule comprises an IL-15v comprising an amino acid sequence of SEQ ID NO: 810. In some embodiments, the CD4-targeted IL-15v molecule comprises an IL-15v comprising an amino acid sequence of SEQ ID NO: 811. In some embodiments, the CD4-targeted IL-15v molecule comprises an IL-15v comprising an amino acid sequence of SEQ ID NO: 812. In some embodiments, the CD4-targeted IL-15v molecule comprises an IL-15v comprising an amino acid sequence of SEQ ID NO: 813. In some embodiments, the CD4-targeted IL-15v molecule comprises an IL-15v comprising an amino acid sequence of SEQ ID NO: 1124. In some embodiments, the CD4-targeted IL-15v molecule comprises an IL-15v comprising an amino acid sequence of SEQ ID NO: 1125.

### IL-15RA Sushi Domain-IL-15v Fusion Polypeptides

In some embodiments, the CD4-targeted IL-15v molecule comprises a fusion protein comprising: (i) an IL-15 receptor alpha subunit (IL15RA) SUSHI domain and (ii) an IL-15 variant (IL-15v) as provided in Table E, e.g., having an amino acid sequence selected from SEQ ID NOs: 814-887, 1126-1127 and 1133-1208. In some embodiments, the CD4-targeted IL-15v molecule comprises an IL-15RA SUSHI domain-IL-15v fusion protein comprising an amino acid sequence of any one of SEQ ID NOs: 817, 824-826, 828-830, 832, 842-845, 848, 851, 853, 857-862, 864-872, 875-882, 884-887 and 1127. In some embodiments, the CD4-targeted IL-15v molecule comprises an IL-15RA SUSHI domain-IL-15v fusion protein comprising an amino acid sequence of any one of SEQ ID NOs: 1136, 1143-1145, 1147-1149, 1151, 1162-1165, 1168, 1171, 1173-1174, 1178-1183, 1185-1193 and 1196-1203, 1205-1208.

In some embodiments, the IL15RA SUSHI domain is N-terminal to the IL-15v. In some embodiments, the IL15RA SUSHI domain binds to IL-15, is no longer than 65 amino acids, and comprises the amino acid sequence of SEQ ID NO: 734, or an amino acid sequence at least 90%, 91%, 92%, 93%, 94%, 95%, 96%, 97%, 98%, 99% identical to SEQ ID NO: 734. In some embodiments, the IL15RA SUSHI domain comprises the amino acid sequence of SEQ ID NO: 735, or an amino acid sequence at least 90%, 91%, 92%, 93%, 94%, 95%, 96%, 97%, 98%, 99% identical to SEQ ID NO: 735. Illustrative IL15RA SUSHI domain sequences are provided in Table C. below. In some embodiments, the IL15RA SUSHI domain comprises an amino acid sequence selected from SEQ ID NOs: 734-739. In some embodiments, the IL15RA SUSHI domain comprises the amino acid sequence of SEQ ID NO: 734. In some embodiments, the IL15RA SUSHI domain comprises the amino acid sequence of SEQ ID NO: 735. In some embodiments, the IL15RA SUSHI domain comprises the amino acid sequence of SEQ ID NO: 736. In some embodiments, the IL15RA SUSHI domain comprises the amino acid sequence of SEQ ID NO: 737. In some embodiments, the IL15RA SUSHI domain comprises the amino acid sequence of SEQ ID NO: 738. In some embodiments, the IL15RA SUSHI domain comprises the amino acid sequence of SEQ ID NO: 739.

In some embodiments, the IL15RA SUSHI domain and IL-15v are connected via a flexible linker, e.g., a Gly-Ser linker. In some embodiments, the linker has a length of from about 4 to about 50 amino acids, e.g., from about 5 amino acids to about 25 amino acids, e.g., from about 15 amino acids to about 25 amino acids. In some embodiments, the linker comprises from 1 to 10 units, e.g., 1 to 5 units, e.g., 3 to 5 units, of a poly-glycine serine linker selected from GGGS (SEQ ID NO: 1128), GGGGS (SEQ ID NO: 1129) and combinations thereof. In some embodiments, the linker comprises 5 units of GGGGS (SEQ ID NO: 1129); (GGGGSGGGGSGGGGSGGGGSGGGGS; SEQ ID NO: 1085).

In some embodiments of the CD4-targeted IL-15v, the fusion protein comprising an IL15RA SUSHI domain and an IL-15v comprises an amino acid sequence of any one of SEQ ID NOs: 814-887, 1126-1127 and 1133-1208. In some embodiments, the fusion protein comprising an IL15RA SUSHI domain and an IL-15v comprises an amino acid sequence of any one of SEQ ID NOs: 817, 824-826, 828-830, 832, 842-845, 848, 851, 853, 857-862, 864-872, 875-882, 884-887 and 1127. In some embodiments, the fusion protein comprising an IL15RA SUSHI domain and an IL-15v comprises an amino acid sequence of SEQ ID NO: 817. In some embodiments, the fusion protein comprising an IL15RA SUSHI domain and an IL-15v comprises an amino acid sequence of SEQ ID NO: 818. In some embodiments, the fusion protein comprising an IL15RA SUSHI domain and an IL-15v comprises an amino acid sequence of SEQ ID NO: 819. In some embodiments, the fusion protein comprising an IL15RA SUSHI domain and an IL-15v comprises an amino acid sequence of SEQ ID NO: 820. In some embodiments, the fusion protein comprising an IL15RA SUSHI domain and an IL-15v comprises an amino acid sequence of SEQ ID NO: 821. In some embodiments, the fusion protein comprising an IL15RA SUSHI domain and an IL-15v comprises an amino acid sequence of SEQ ID NO: 822. In some embodiments, the fusion protein comprising an IL15RA SUSHI domain and an IL-15v comprises an amino acid sequence of SEQ ID NO: 823. In some embodiments, the fusion protein comprising an IL15RA SUSHI domain and an IL-15v comprises an amino acid sequence of SEQ ID NO: 824. In some embodiments, the fusion protein comprising an IL15RA SUSHI domain and an IL-15v comprises an amino acid sequence of SEQ ID NO: 825. In some embodiments, the fusion protein comprising an IL15RA SUSHI domain and an IL-15v comprises an amino acid sequence of SEQ ID NO: 826. In some embodiments, the fusion protein comprising an IL15RA SUSHI domain and an IL-15v comprises an amino acid sequence of SEQ ID NO: 827. In some embodiments, the fusion protein comprising an IL15RA SUSHI domain and an IL-15v comprises an amino acid sequence of SEQ ID NO: 828. In some embodiments, the fusion protein comprising an IL15RA SUSHI domain and an IL-15v comprises an amino acid sequence of SEQ ID NO: 829. In some embodiments, the fusion protein comprising an IL15RA SUSHI domain and an IL-15v comprises an amino acid sequence of SEQ ID NO: 830. In some embodiments, the fusion protein comprising an IL15RA SUSHI domain and an IL-15v comprises an amino acid sequence of SEQ ID NO: 831. In some embodiments, the fusion protein comprising an IL15RA SUSHI domain and an IL-15v comprises an amino acid sequence of SEQ ID NO: 832. In some embodiments, the fusion protein comprising an IL15RA SUSHI domain and an IL-15v comprises an amino acid sequence of SEQ ID NO: 833. In some embodiments, the fusion protein comprising an IL15RA SUSHI domain and an IL-15v comprises an amino acid sequence of SEQ ID NO: 834. In some embodiments, the fusion protein comprising an IL15RA SUSHI domain and an IL-15v comprises an amino acid sequence of SEQ ID NO: 835. In some embodiments, the fusion protein comprising an IL15RA SUSHI domain and an IL-15v comprises an amino acid sequence of SEQ ID NO: 836. In some embodiments, the fusion protein comprising an IL15RA SUSHI domain and an IL-15v comprises an amino acid sequence of SEQ ID NO: 837. In some embodiments, the fusion protein comprising an IL15RA SUSHI domain and an IL-15v comprises an amino acid sequence of SEQ ID NO: 838. In some embodiments, the fusion protein comprising an IL15RA SUSHI domain and an IL-15v comprises an amino acid sequence of SEQ ID NO: 839. In some embodiments, the fusion protein comprising an IL15RA SUSHI domain and an IL-15v comprises an amino acid sequence of SEQ ID NO: 840. In some embodiments, the fusion protein comprising an IL15RA SUSHI domain and an IL-15v comprises an amino acid sequence of SEQ ID NO: 841. In some embodiments, the fusion protein comprising an IL15RA SUSHI domain and an IL-15v comprises an amino acid sequence of SEQ ID NO: 842. In some embodiments, the fusion protein comprising an IL15RA SUSHI domain and an IL-15v comprises an amino acid sequence of SEQ ID NO: 843. In some embodiments, the fusion protein comprising an IL15RA SUSHI domain and an IL-15v comprises an amino acid sequence of SEQ ID NO: 844. In some embodiments, the fusion protein comprising an IL15RA SUSHI domain and an IL-15v comprises an amino acid sequence of SEQ ID NO: 845. In some embodiments, the fusion protein comprising an IL15RA SUSHI domain and an IL-15v comprises an amino acid sequence of SEQ ID NO: 846. In some embodiments, the fusion protein comprising an IL15RA SUSHI domain and an IL-15v comprises an amino acid sequence of SEQ ID NO: 847. In some embodiments, the fusion protein comprising an IL15RA SUSHI domain and an IL-15v comprises an amino acid sequence of SEQ ID NO: 848. In some embodiments, the fusion protein comprising an IL15RA SUSHI domain and an IL-15v comprises an amino acid sequence of SEQ ID NO: 849. In some embodiments, the fusion protein comprising an IL15RA SUSHI domain and an IL-15v comprises an amino acid sequence of SEQ ID NO: 850. In some embodiments, the fusion protein comprising an IL15RA SUSHI domain and an IL-15v comprises an amino acid sequence of SEQ ID NO: 851. In some embodiments, the fusion protein comprising an IL15RA SUSHI domain and an IL-15v comprises an amino acid sequence of SEQ ID NO: 852. In some embodiments, the fusion protein comprising an IL15RA SUSHI domain and an IL-15v comprises an amino acid sequence of SEQ ID NO: 853. In some embodiments, the fusion protein comprising an IL15RA SUSHI domain and an IL-15v comprises an amino acid sequence of SEQ ID NO: 854. In some embodiments, the fusion protein comprising an IL15RA SUSHI domain and an IL-15v comprises an amino acid sequence of SEQ ID NO: 855. In some embodiments, the fusion protein comprising an IL15RA SUSHI domain and an IL-15v comprises an amino acid sequence of SEQ ID NO: 856. In some embodiments, the fusion protein comprising an IL15RA SUSHI domain and an IL-15v comprises an amino acid sequence of SEQ ID NO: 857. In some embodiments, the fusion protein comprising an IL15RA SUSHI domain and an IL-15v comprises an amino acid sequence of SEQ ID NO: 858. In some embodiments, the fusion protein comprising an IL15RA SUSHI domain and an IL-15v comprises an amino acid sequence of SEQ ID NO: 859. In some embodiments, the fusion protein comprising an IL15RA SUSHI domain and an IL-15v comprises an amino acid sequence of SEQ ID NO: 860. In some embodiments, the fusion protein comprising an IL15RA SUSHI domain and an IL-15v comprises an amino acid sequence of SEQ ID NO: 861. In some embodiments, the fusion protein comprising an IL15RA SUSHI domain and an IL-15v comprises an amino acid sequence of SEQ ID NO: 862. In some embodiments, the fusion protein comprising an IL15RA SUSHI domain and an IL-15v comprises an amino acid sequence of SEQ ID NO: 863. In some embodiments, the fusion protein comprising an IL15RA SUSHI domain and an IL-15v comprises an amino acid sequence of SEQ ID NO: 864. In some embodiments, the fusion protein comprising an IL15RA SUSHI domain and an IL-15v comprises an amino acid sequence of SEQ ID NO: 865. In some embodiments, the fusion protein comprising an IL15RA SUSHI domain and an IL-15v comprises an amino acid sequence of SEQ ID NO: 866. In some embodiments, the fusion protein comprising an IL15RA SUSHI domain and an IL-15v comprises an amino acid sequence of SEQ ID NO: 867. In some embodiments, the fusion protein comprising an IL15RA SUSHI domain and an IL-15v comprises an amino acid sequence of SEQ ID NO: 868. In some embodiments, the fusion protein comprising an IL15RA SUSHI domain and an IL-15v comprises an amino acid sequence of SEQ ID NO: 869. In some embodiments, the fusion protein comprising an IL15RA SUSHI domain and an IL-15v comprises an amino acid sequence of SEQ ID NO: 870. In some embodiments, the fusion protein comprising an IL15RA SUSHI domain and an IL-15v comprises an amino acid sequence of SEQ ID NO: 871. In some embodiments, the fusion protein comprising an IL15RA SUSHI domain and an IL-15v comprises an amino acid sequence of SEQ ID NO: 872. In some embodiments, the fusion protein comprising an IL15RA SUSHI domain and an IL-15v comprises an amino acid sequence of SEQ ID NO: 873. In some embodiments, the fusion protein comprising an IL15RA SUSHI domain and an IL-15v comprises an amino acid sequence of SEQ ID NO: 874. In some embodiments, the fusion protein comprising an IL15RA SUSHI domain and an IL-15v comprises an amino acid sequence of SEQ ID NO: 875. In some embodiments, the fusion protein comprising an IL15RA SUSHI domain and an IL-15v comprises an amino acid sequence of SEQ ID NO: 876. In some embodiments, the fusion protein comprising an IL15RA SUSHI domain and an IL-15v comprises an amino acid sequence of SEQ ID NO: 877. In some embodiments, the fusion protein comprising an IL15RA SUSHI domain and an IL-15v comprises an amino acid sequence of SEQ ID NO: 878. In some embodiments, the fusion protein comprising an IL15RA SUSHI domain and an IL-15v comprises an amino acid sequence of SEQ ID NO: 879. In some embodiments, the fusion protein comprising an IL15RA SUSHI domain and an IL-15v comprises an amino acid sequence of SEQ ID NO: 880. In some embodiments, the fusion protein comprising an IL15RA SUSHI domain and an IL-15v comprises an amino acid sequence of SEQ ID NO: 881. In some embodiments, the fusion protein comprising an IL15RA SUSHI domain and an IL-15v comprises an amino acid sequence of SEQ ID NO: 882. In some embodiments, the fusion protein comprising an IL15RA SUSHI domain and an IL-15v comprises an amino acid sequence of SEQ ID NO: 883. In some embodiments, the fusion protein comprising an IL15RA SUSHI domain and an IL-15v comprises an amino acid sequence of SEQ ID NO: 884. In some embodiments, the fusion protein comprising an IL15RA SUSHI domain and an IL-15v comprises an amino acid sequence of SEQ ID NO: 885. In some embodiments, the fusion protein comprising an IL15RA SUSHI domain and an IL-15v comprises an amino acid sequence of SEQ ID NO: 886. In some embodiments, the fusion protein comprising an IL15RA SUSHI domain and an IL-15v comprises an amino acid sequence of SEQ ID NO: 887. In some embodiments, the fusion protein comprising an IL15RA SUSHI domain and an IL-15v comprises an amino acid sequence of SEQ ID NO: 1127.

In some embodiments of the CD4-targeted IL-15v, the fusion protein comprising an IL15RA SUSHI domain and an IL-15v comprises an amino acid sequence of any one of SEQ ID NOs: 1133-1208. In some embodiments, the fusion protein comprising an IL15RA SUSHI domain and an IL-15v comprises an amino acid sequence of any one of SEQ ID NOs: 1136, 1143-1145, 1147-1149, 1151, 1162-1165, 1168, 1171, 1173-1174, 1178-1183, 1185-1193 and 1196-1203, 1205-1208. In some embodiments, the fusion protein comprising an IL15RA SUSHI domain and an IL-15v comprises an amino acid sequence of SEQ ID NO: 1133. In some embodiments, the fusion protein comprising an IL15RA SUSHI domain and an IL-15v comprises an amino acid sequence of SEQ ID NO: 1134. In some embodiments, the fusion protein comprising an IL15RA SUSHI domain and an IL-15v comprises an amino acid sequence of SEQ ID NO: 1135. In some embodiments, the fusion protein comprising an IL15RA SUSHI domain and an IL-15v comprises an amino acid sequence of SEQ ID NO: 1136. In some embodiments, the fusion protein comprising an IL15RA SUSHI domain and an IL-15v comprises an amino acid sequence of SEQ ID NO: 1137. In some embodiments, the fusion protein comprising an IL15RA SUSHI domain and an IL-15v comprises an amino acid sequence of SEQ ID NO: 1138. In some embodiments, the fusion protein comprising an IL15RA SUSHI domain and an IL-15v comprises an amino acid sequence of SEQ ID NO: 1139. In some embodiments, the fusion protein comprising an IL15RA SUSHI domain and an IL-15v comprises an amino acid sequence of SEQ ID NO: 1140. In some embodiments, the fusion protein comprising an IL15RA SUSHI domain and an IL-15v comprises an amino acid sequence of SEQ ID NO: 1141. In some embodiments, the fusion protein comprising an IL15RA SUSHI domain and an IL-15v comprises an amino acid sequence of SEQ ID NO: 1142. In some embodiments, the fusion protein comprising an IL15RA SUSHI domain and an IL-15v comprises an amino acid sequence of SEQ ID NO: 1143. In some embodiments, the fusion protein comprising an IL15RA SUSHI domain and an IL-15v comprises an amino acid sequence of SEQ ID NO: 1144. In some embodiments, the fusion protein comprising an IL15RA SUSHI domain and an IL-15v comprises an amino acid sequence of SEQ ID NO: 1145. In some embodiments, the fusion protein comprising an IL15RA SUSHI domain and an IL-15v comprises an amino acid sequence of SEQ ID NO: 1146. In some embodiments, the fusion protein comprising an IL15RA SUSHI domain and an IL-15v comprises an amino acid sequence of SEQ ID NO: 1147. In some embodiments, the fusion protein comprising an IL15RA SUSHI domain and an IL-15v comprises an amino acid sequence of SEQ ID NO: 1148. In some embodiments, the fusion protein comprising an IL15RA SUSHI domain and an IL-15v comprises an amino acid sequence of SEQ ID NO: 1149. In some embodiments, the fusion protein comprising an IL15RA SUSHI domain and an IL-15v comprises an amino acid sequence of SEQ ID NO: 1150. In some embodiments, the fusion protein comprising an IL15RA SUSHI domain and an IL-15v comprises an amino acid sequence of SEQ ID NO: 1151. In some embodiments, the fusion protein comprising an IL15RA SUSHI domain and an IL-15v comprises an amino acid sequence of SEQ ID NO: 1152. In some embodiments, the fusion protein comprising an IL15RA SUSHI domain and an IL-15v comprises an amino acid sequence of SEQ ID NO: 1153. In some embodiments, the fusion protein comprising an IL15RA SUSHI domain and an IL-15v comprises an amino acid sequence of SEQ ID NO: 1154. In some embodiments, the fusion protein comprising an IL15RA SUSHI domain and an IL-15v comprises an amino acid sequence of SEQ ID NO: 1155. In some embodiments, the fusion protein comprising an IL15RA SUSHI domain and an IL-15v comprises an amino acid sequence of SEQ ID NO: 1156. In some embodiments, the fusion protein comprising an IL15RA SUSHI domain and an IL-15v comprises an amino acid sequence of SEQ ID NO: 1157. In some embodiments, the fusion protein comprising an IL15RA SUSHI domain and an IL-15v comprises an amino acid sequence of SEQ ID NO: 1158. In some embodiments, the fusion protein comprising an IL15RA SUSHI domain and an IL-15v comprises an amino acid sequence of SEQ ID NO: 1159. In some embodiments, the fusion protein comprising an IL15RA SUSHI domain and an IL-15v comprises an amino acid sequence of SEQ ID NO: 1160. In some embodiments, the fusion protein comprising an IL15RA SUSHI domain and an IL-15v comprises an amino acid sequence of SEQ ID NO: 1161. In some embodiments, the fusion protein comprising an IL15RA SUSHI domain and an IL-15v comprises an amino acid sequence of SEQ ID NO: 1162. In some embodiments, the fusion protein comprising an IL15RA SUSHI domain and an IL-15v comprises an amino acid sequence of SEQ ID NO: 1163. In some embodiments, the fusion protein comprising an IL15RA SUSHI domain and an IL-15v comprises an amino acid sequence of SEQ ID NO: 1164. In some embodiments, the fusion protein comprising an IL15RA SUSHI domain and an IL-15v comprises an amino acid sequence of SEQ ID NO: 1165. In some embodiments, the fusion protein comprising an IL15RA SUSHI domain and an IL-15v comprises an amino acid sequence of SEQ ID NO: 1166. In some embodiments, the fusion protein comprising an IL15RA SUSHI domain and an IL-15v comprises an amino acid sequence of SEQ ID NO: 1167. In some embodiments, the fusion protein comprising an IL15RA SUSHI domain and an IL-15v comprises an amino acid sequence of SEQ ID NO: 1168. In some embodiments, the fusion protein comprising an IL15RA SUSHI domain and an IL-15v comprises an amino acid sequence of SEQ ID NO: 1169. In some embodiments, the fusion protein comprising an IL15RA SUSHI domain and an IL-15v comprises an amino acid sequence of SEQ ID NO: 1170. In some embodiments, the fusion protein comprising an IL15RA SUSHI domain and an IL-15v comprises an amino acid sequence of SEQ ID NO: 1171. In some embodiments, the fusion protein comprising an IL15RA SUSHI domain and an IL-15v comprises an amino acid sequence of SEQ ID NO: 1172. In some embodiments, the fusion protein comprising an IL15RA SUSHI domain and an IL-15v comprises an amino acid sequence of SEQ ID NO: 1173. In some embodiments, the fusion protein comprising an IL15RA SUSHI domain and an IL-15v comprises an amino acid sequence of SEQ ID NO: 1174. In some embodiments, the fusion protein comprising an IL15RA SUSHI domain and an IL-15v comprises an amino acid sequence of SEQ ID NO: 1175. In some embodiments, the fusion protein comprising an IL15RA SUSHI domain and an IL-15v comprises an amino acid sequence of SEQ ID NO: 1176. In some embodiments, the fusion protein comprising an IL15RA SUSHI domain and an IL-15v comprises an amino acid sequence of SEQ ID NO: 1177. In some embodiments, the fusion protein comprising an IL15RA SUSHI domain and an IL-15v comprises an amino acid sequence of SEQ ID NO: 1178. In some embodiments, the fusion protein comprising an IL15RA SUSHI domain and an IL-15v comprises an amino acid sequence of SEQ ID NO: 1179. In some embodiments, the fusion protein comprising an IL15RA SUSHI domain and an IL-15v comprises an amino acid sequence of SEQ ID NO: 1180. In some embodiments, the fusion protein comprising an IL15RA SUSHI domain and an IL-15v comprises an amino acid sequence of SEQ ID NO: 1181. In some embodiments, the fusion protein comprising an IL15RA SUSHI domain and an IL-15v comprises an amino acid sequence of SEQ ID NO: 1182. In some embodiments, the fusion protein comprising an IL15RA SUSHI domain and an IL-15v comprises an amino acid sequence of SEQ ID NO: 1183. In some embodiments, the fusion protein comprising an IL15RA SUSHI domain and an IL-15v comprises an amino acid sequence of SEQ ID NO: 1184. In some embodiments, the fusion protein comprising an IL15RA SUSHI domain and an IL-15v comprises an amino acid sequence of SEQ ID NO: 1185. In some embodiments, the fusion protein comprising an IL15RA SUSHI domain and an IL-15v comprises an amino acid sequence of SEQ ID NO: 1186. In some embodiments, the fusion protein comprising an IL15RA SUSHI domain and an IL-15v comprises an amino acid sequence of SEQ ID NO: 1187. In some embodiments, the fusion protein comprising an IL15RA SUSHI domain and an IL-15v comprises an amino acid sequence of SEQ ID NO: 1188. In some embodiments, the fusion protein comprising an IL15RA SUSHI domain and an IL-15v comprises an amino acid sequence of SEQ ID NO: 1189. In some embodiments, the fusion protein comprising an IL15RA SUSHI domain and an IL-15v comprises an amino acid sequence of SEQ ID NO: 1190. In some embodiments, the fusion protein comprising an IL15RA SUSHI domain and an IL-15v comprises an amino acid sequence of SEQ ID NO: 1191. In some embodiments, the fusion protein comprising an IL15RA SUSHI domain and an IL-15v comprises an amino acid sequence of SEQ ID NO: 1192. In some embodiments, the fusion protein comprising an IL15RA SUSHI domain and an IL-15v comprises an amino acid sequence of SEQ ID NO: 1193. In some embodiments, the fusion protein comprising an IL15RA SUSHI domain and an IL-15v comprises an amino acid sequence of SEQ ID NO: 1194. In some embodiments, the fusion protein comprising an IL15RA SUSHI domain and an IL-15v comprises an amino acid sequence of SEQ ID NO: 1195. In some embodiments, the fusion protein comprising an IL15RA SUSHI domain and an IL-15v comprises an amino acid sequence of SEQ ID NO: 1196. In some embodiments, the fusion protein comprising an IL15RA SUSHI domain and an IL-15v comprises an amino acid sequence of SEQ ID NO: 1197. In some embodiments, the fusion protein comprising an IL15RA SUSHI domain and an IL-15v comprises an amino acid sequence of SEQ ID NO: 1198. In some embodiments, the fusion protein comprising an IL15RA SUSHI domain and an IL-15v comprises an amino acid sequence of SEQ ID NO: 1199. In some embodiments, the fusion protein comprising an IL15RA SUSHI domain and an IL-15v comprises an amino acid sequence of SEQ ID NO: 1200. In some embodiments, the fusion protein comprising an IL15RA SUSHI domain and an IL-15v comprises an amino acid sequence of SEQ ID NO: 1201. In some embodiments, the fusion protein comprising an IL15RA SUSHI domain and an IL-15v comprises an amino acid sequence of SEQ ID NO: 1202. In some embodiments, the fusion protein comprising an IL15RA SUSHI domain and an IL-15v comprises an amino acid sequence of SEQ ID NO: 1203. In some embodiments, the fusion protein comprising an IL15RA SUSHI domain and an IL-15v comprises an amino acid sequence of SEQ ID NO: 1204. In some embodiments, the fusion protein comprising an IL15RA SUSHI domain and an IL-15v comprises an amino acid sequence of SEQ ID NO: 1205. In some embodiments, the fusion protein comprising an IL15RA SUSHI domain and an IL-15v comprises an amino acid sequence of SEQ ID NO: 1206. In some embodiments, the fusion protein comprising an IL15RA SUSHI domain and an IL-15v comprises an amino acid sequence of SEQ ID NO: 1207. In some embodiments, the fusion protein comprising an IL15RA SUSHI domain and an IL-15v comprises an amino acid sequence of SEQ ID NO: 1208.

d. Fc Domains or Fc Regions

In some embodiments, the first Fc domain and a second Fc domain (or a first Fc region and a second Fc region) are IgG4. In some embodiments, the first Fc domain and a second Fc domain (or a first Fc region and a second Fc region) are IgG1.

### Heterodimerization Facilitation or Promotion

Generally, the heterodimers have a first Fc domain and a second Fc domain (or a first Fc region and a second Fc region), wherein the first Fc domain and the second Fc domain are different. In order to facilitate heterodimerization, in some embodiments, the first Fc domain and the second Fc domain can comprise amino acid the following amino acid substitutions (Eu numbering), respectively: T366W and T366S/L368A/Y407V; T366S/L368A/Y407V and T366W; T366W/S354C and T366S/L368A/Y407V/Y349C; T366S/L368A/Y407V/Y349C and T366W/S354C; S364H/F405A and Y349T/T394F; Y349T/T394F and S364H/F405A; T350V/L351Y/F405A/Y407V and T350V/T366L/K392L/T394W; T350V/T366L/K392L/T394W and T350V/L351Y/F405A/Y407V; K360D/D399M/Y407A and E345R/Q347R/T366V/K409V; E345R/Q347R/T366V/K409V and K360D/D399M/Y407A; K409D/K392D and D399K/E356K; D399K/E356K and K409D/K392D; K360E/K409W and Q347R/D399V/F405T; Q347R/D399V/F405T and K360E/K409W; K360E/K409W/Y349C and Q347R/D399V/F405T/S354C; Q347R/D399V/F405T/S354C and K360E/K409W/Y349C; K370E/K409W and E357N/D399V/F405T; or E357N/D399V/F405T and K370E/K409W.

In order to facilitate heterodimerization, in some embodiments, the first Fc domain and the second Fc domain comprise amino acid the following amino acid substitutions (Eu numbering), respectively: the first Fc domain comprises a tryptophan at position 366 (T366W); and the second Fc domain comprises a serine at position 366 (T366S), an alanine at position 368 (L368A) and a valine at position 407 (Y407V); the first Fc domain comprises a cysteine at position 354 (S354C), a tryptophan at position 366 (T366W); and the second Fc domain comprises a cysteine at position 349 (Y349C), a serine at position 366 (T366S), an alanine at position 368 (L368A) and a valine at position 407 (Y407V); the first Fc domain comprises a glutamic acid at position 360 (K360E) and a tryptophan at position 409 (K409W) and the second Fc domain comprises a valine at position 399 (D399V) and a threonine at position 405 (F405T); the first Fc domain comprises a serine at position 349 (Y349S) and the second Fc domain comprises a tryptophan at position 357 (E357W); the first Fc domain comprises a lysine at position 356 (E356K), a lysine at position 357 (E357K) and a lysine at position 399 (D399K) and the second Fc domain comprises glutamic acid at position 370 (K370E), an aspartic acid at position 409 (K409D) and a glutamic acid at position 439 (K439E); the first Fc domain comprises an aspartic acid at position 392 (K392D) and an aspartic acid at position 409 (K409D) and the second Fc domain comprises a lysine at position 356 (E356K) and a lysine at position 399 (D399K); the first Fc domain comprises a glutamic acid at position 364 (S364E) and an alanine at position 405 (F405A) and the second Fc domain comprises a lysine at position 349 (Y349K) and a phenylalanine at position 394 (T394F); the first Fc domain comprises a histidine at position 364 (S364H) and a lysine at position 401 (D401K) and the second Fc domain comprises a threonine at position 349 (Y349T) and a glutamic acid at position 411 (T411E); the first Fc domain comprises a histidine at position 364 (S364H) and a phenylalanine at position 394 (T394F) and the second Fc domain comprises a threonine at position 349 (Y349T) and an alanine at position 405 (F405A); the first Fc domain comprises a glutamic acid at position 364 (S364E) and a phenylalanine at position 394 (T394F) and the second Fc domain comprises a lysine at position 349 (Y349K) and an alanine at position 405 (F405A); the first Fc domain comprises a glutamic acid at position 364 (S364E) and a glutamic acid at position 411 (T411E) and the second Fc domain comprises a lysine at position 349 (Y349K) and a lysine at position 401 (D401K); the first Fc domain comprises an aspartic acid at position 364 (S364D) and a phenylalanine at position 394 (T394F) and the second Fc domain comprises a lysine at position 349 (Y349K) and an alanine at position 405 (F405A); the first Fc domain comprises a histidine at position 364 (S364H) and an alanine at position 405 (F405A) and the second Fc domain comprises a threonine at position 349 (Y349T) and a phenylalanine at position 394 (T394F); the first Fc domain comprises a lysine at position 364 (S364K) and a glutamine at position 357 (E357Q) and the second Fc domain comprises an aspartic acid at position 368 (L368D) and a serine at position 370 (K370); the first Fc domain comprises a tyrosine at position 351 (L351Y), an alanine at position 405 (F405A), and a valine at position 407 (Y407V), and the second Fc domain comprises a leucine at position 366 (T366L), a methionine at position 392 (K392M), and a tryptophan at position 394 (T394W); the first Fc domain comprises a tyrosine at position 351 (L351Y), an alanine at position 405 (F405A), and a valine at position 407 (Y407V), and the second Fc domain comprises a leucine at position 366 (T366L), a leucine at position 392 (K392L), and a tryptophan at position 394 (T394W); the first Fc domain comprises a valine at position 350 (T350V), a tyrosine at position 351 (L351Y), an alanine at position 405 (F405A), and a valine at position 407 (Y407V), and the second Fc domain comprises a valine at position 350 (T350V), a leucine at position 366 (T366L), a methionine at position 392 (K392M), and a tryptophan at position 394 (T394W); the first Fc domain comprises a valine at position 350 (T350V), a tyrosine at position 351 (L351Y), an alanine at position 405 (F405A), and a valine at position 407 (Y407V), and the second Fc domain comprises a valine at position 350 (T350V), a leucine at position 366 (T366L), a leucine at position 392 (K392L), and a tryptophan at position 394 (T394W); the first Fc domain comprises a leucine at position 405 (F405L) and the second Fc domain comprises an arginine at position 409 (K409R); the first Fc domain comprises a lysine at position 405 (F405K) and the second Fc domain comprises an alanine at position 409 (K409A); the first Fc domain comprises a lysine at position 356 (D356K), a lysine at position 399 (D399K) and an alanine at position 407 (Y407A) and the second Fc domain comprises an aspartic acid at position 392 (K392D) and a tryptophan at position 366 (T366W); or the first Fc domain comprises an aspartic acid at position 351 (L351D), a glutamic acid at position 368 (L368E) and the second Fc domain comprises a lysine at position 351 (L351K) and a lysine at position threonine (T366K). See, e.g., U.S. Pat. Nos. 5,731,168; 7,642,228; 7,695,936; 8,216,805; 8,679,785; 10,899,846; 7,183,076; 9,637,557; 9,862,778; 9,688,775; 8,592,562; 9,200,060; 11,198,732; 9,951,145; 11,286,311; 9,493,578; 9,650,446; 10,858,417; 11,401,348; 9,499,634; 10,508,154; 9,562,109; 9,574,010; 10,457,742; 10,875,931; 10,138,303; 9,527,926; 9,150,663; 10,597,464; 10,344,050; 9,683,052; 9,683,053; 10,011,858; 11,168,344; 10,538,595; 11,168,149; 9,248,181; 9,248,182; 9,358,286; 9,758,805; 10,329,596; 10,752,929; 8,871,912; 9,505,848; 9,714,291; 10,494,437; and 11,059,911, which are hereby incorporated herein by reference in their entireties for all purposes.

### Serum Half-Life Extension

To promote extended serum half-life of the heterodimer, in some embodiments, one or both of the first Fc domain and the second Fc domain comprise amino acid modifications that promote an increased serum half-life of the anti-binding molecule. Amino acid substitutions that increase the half-life of an antibody have been described. In one embodiment, the Fc region or Fc domain of one or both of heavy chains comprise a methionine to tyrosine substitution at position 252 (Eu numbering), a serine to threonine substitution at position 254 (Eu numbering), and a threonine to glutamic acid substitution at position 256 (Eu numbering). See, e.g., U.S. Pat. No. 7,658,921. This type of mutant, designated as a “YTE” exhibits a four-fold increased half-life relative to wild-type versions of the same antibody (Dall'Acqua, et al., J Biol Chem, 281:23514-24 (2006); Robbie, et al., Antimicrob Agents Chemotherap., 57(12):6147-6153 (2013)). In certain embodiments, the Fc region or Fc domain of one or both heavy chains comprise an IgG constant domain comprising one, two, three or more amino acid substitutions of amino acid residues at positions 251-257, 285-290, 308-314, 385-389, and 428-436 (Eu numbering). Alternatively, M428L and N434S (“LS”) amino acid substitutions can increase the pharmacokinetic half-life of the CD4-targeted IL-15v molecule. In other embodiments, the Fc region or Fc domain of one or both heavy chains comprise a M428L and N434S substitution (Eu numbering). In other embodiments, the Fc region or Fc domain of one or both heavy chains comprise T250Q and M428L (Eu numbering) amino acid substitutions, e.g., as described in U.S. Pat. Nos. 7,217,797 and 7,217,798. In other embodiments, the Fc region or Fc domain of one or both heavy chains comprise H433K and N434F (Eu numbering) amino acid substitutions, e.g., as described in U.S. Pat. No. 8,163,881. In other embodiments, the Fc region or Fc domain of one or both heavy chains comprise T307Q/Q311V/A378V (DF215) or T256D/N286D/T307R/Q311V/A378V (DF228) (Eu numbering) amino acid substitutions, e.g., as described in U.S. Patent Publ. No. 2020-0277358. In some embodiments, the Fc region or Fc domain of one or both heavy chains comprise aspartic acid at position 309, histidine at position 311 and serine at position 434 (DHS), e.g., as described in U.S. Pat. No. 11,059,892. In some embodiments, one or both of the first Fc domain and the second Fc domain comprises serum half-life extending amino acid substitutions at Fc positions (Eu numbering) selected from: tyrosine at position 252, threonine at position 254 and glutamic acid at position 256 (252Y, 254T and 256E); leucine at position 428 (428L); glutamine at position 250 and leucine at position 428 (250Q and 428L); leucine at position 428 and serine at position 434 (428L and 434S); leucine at position 428 and alanine at position 434 (428L and 434A); arginine at position 311 and leucine at position 428 (311R and 428L); glycine at position 309 and leucine at position 428 (309G and 428L); glutamine at position 307, valine at position 311 and valine at position 378 (307Q, 311V and 378V); aspartic acid at position 256, aspartic acid at position 286, arginine at position 307, valine at position 311 and valine at position 378 (256D, 286D, 307R, 311V and 378V). See, e.g., U.S. Pat. Nos. 6,821,505; 6,277,375; 7,083,784; 8,475,792; 7,217,797; 7,217,798; 7,670,600; 8,088,376; 8,394,925; 8,546,543; 9,803,023; 10,336,818; 10,683,368; 11,440,971; 11,059,892; 8,163,881; 8,834,871; 11,492,415; and 11,319,383; and U.S. Patent Publication Nos. 2019/0292269; 2020/0277358; and 2020/0277358, which are hereby incorporated herein by reference in their entireties for all purposes.

In some embodiments, the herein described CD4-targeted IL-15v do not have any serum half-life extending amino acid substitutions in the first and second Fc domains.

### Protein A Affinity Purification

To facilitate protein A purification of the heterodimer, in some embodiments, either the first Fc domain or the second Fc domain comprise an amino acid substitution at one or both of positions H435 and Y436 (EU index numbering). In some embodiments, either the first Fc domain or the second Fc domain comprise an arginine at position 435 (EU index numbering). In some embodiments, either the first Fc domain or the second Fc domain comprise an arginine at position 435 (H435R) and a phenylalanine or an alanine at position 436 (Y436F or Y436A) (EU index numbering). In some embodiments, either the first Fc domain or the second Fc domain comprise Q311R, Q311K, T307P/L309Q, or T307P/L309Q/Q311R (EU index numbering). In some embodiments, either the first Fc domain or the second Fc domain an arginine at position 435 and a phenylalanine at position 436 and the T366S/L368A/Y407V amino acid substitutions. See, e.g., Jendeberg, et al., J Immunol Methods (1997) 201(1):25-34 and U.S. Pat. Nos. 11,168,111; 9,670,269; 11,555,067; and 11,149,094, which are hereby incorporated herein by reference in their entireties for all purposes.

### Effector Function Modulation

In some embodiments, the effector functions of one or both Fc domains of the heterodimer are reduced or eliminated. In some embodiments, one or both of the first Fc domain and the second Fc domain comprise a human IgG4 isotype and comprises one or more amino acid substitutions in the Fc domain or Fc region at a residue position selected from the group consisting of: F234V, F234A, L235A, L235E, S228P, and any combination thereof, wherein the numbering of the residues is according to Eu numbering. In some embodiments, one or both of the first Fc domain and the second Fc domain comprise a human IgG1 isotype and comprises one or more amino acid substitutions in the Fc region at a residue position selected from the group consisting of: L234A, L234V, L234F, L235A, L235E, P331S, and any combination thereof, wherein the numbering of the residues is according to Eu numbering. In some embodiments, one or both of the first Fc domain and the second Fc domain comprises effector function reducing amino acid substitutions at Fc positions (Eu numbering) selected from: alanine at position 234 and alanine at position 235 (234A and 235A); glutamic acid at position 235 (235E); proline at position 228 and glutamic acid at position 235 (228P, 235E); alanine at position 237 (237A); alanine at position 234 and glutamic acid at position 235 (234A and 235E); phenylalanine at position 234, glutamic acid at position 235 and alanine at position 265 (234F, 235E and 265A); phenylalanine at position 234, glutamic acid at position 235 and alanine at position 236 (234F, 235E and 236A); phenylalanine at position 234, glutamic acid at position 235 and arginine at position 236 (234F, 235E and 236R); serine at position 234, threonine at position 235 and arginine at position 236 (234S, 235T and 236R); alanine at position 234, alanine at position 235 and glycine at position 329 (234A, 235A and 329G); alanine at position 234, alanine at position 235 and alanine at position 237 (234A, 235A and 237A); proline at position 228 and glutamic acid at position 235 (228P and 235E); proline at position 228, glutamic acid in position 235 and glycine at position 329 (228P, 235E and 329G); phenylalanine at position 234, glutamic acid at position 235 and serine at position 331 (234F, 235E and 331S); phenylalanine at position 234, glutamine at position 235 and glutamine at position 322 (234F, 235Q and 322Q); alanine at position 233, arginine at position 234 and arginine at position 235 (233A, 234R and 235R); alanine at position 233, aspartic acid at position 234 and glutamic acid at position 235 (233A, 234D and 235E); lysine at position 233, arginine at position 234 and arginine at position 235 (233K, 234R and 235R); alanine at position 234, alanine at position 235, alanine at position 237, serine at position 238, alanine at position 268, serine at position 330 and serine at position 331 (234A, 235A, 237A, 238S, 268A, 330S and 331S); and proline at position 228, alanine at position 234, alanine at position 235, alanine at position 237 and serine at position 238 (228P, 234A, 235A, 237A and 238S). In some embodiments, the first and second Fc domain are IgG1 and one or both of the first Fc domain and the second Fc domain comprises one or more amino acid substitutions in the Fc region at a residue position selected from the group consisting of: L234A, L234V, L234F, L235A, L235E, G237A, P331S, and any combination thereof, wherein the numbering of the residues is according to Eu numbering. In some embodiments, the first and second Fc domain are IgG4 and one or both of the first Fc domain and the second Fc domain comprises one or more amino acid substitutions in the Fc region at a residue position selected from the group consisting of: F234V, F234A, L235A, L235E, G237A, S228P, and any combination thereof, wherein the numbering of the residues is according to Eu numbering. See, e.g., U.S. Pat. Nos. 5,624,821; 5,648,260; 6,491,916; 8,409,568; 8,969,526; 5,885,573; 10,590,206; 10,011,660; 11,098,105; 9,790,268; 10,836,813; 10,562,950; 10,093,711; 9,546,203; 7,863,419; 7,662,925; 8,093,357; 8,679,493; 8,399,618; 8,883,147; 7,332,581; 7,122,637; 10,053,513; 10,894,836; 9,637,549; 8,961,967; 10,053,513; 10,526,408; 9,718,893; 9,228,017; 10,066,018; 9,890,218; 10,766,960; 11,267,868; 9,688,762; and 11,332,533; U.S. Patent Publication No. 2020/181257 and Intl. Publication Nos. WO 2022/189667; WO 2021/234402; WO 2022/044248; WO 2022/045276, which are hereby incorporated herein by reference in their entireties for all purposes.

In some embodiments, the effector functions of one or both Fc domains of the heterodimer are increased or enhanced. In some embodiments, one or both of the first Fc domain and the second Fc domain comprises one or more amino acid substitutions in the Fc domain or Fc region at a residue position selected from the group consisting of aspartic acid at position 239, glutamic acid at position 332, alanine at position 236, leucine at position 330, and any combination thereof, wherein the numbering of the residues is according to Eu numbering. In some embodiments, one or both of the first Fc domain and the second Fc domain comprises effector function enhancing amino acid substitutions at Fc positions (Eu numbering) selected from: aspartic acid at position 239 and glutamic acid at position 332 (239D and 332E); aspartic acid at position 239, glutamic acid at position 332 and leucine at position 330 (239D, 332E and 330L); aspartic acid at position 239, glutamic acid at position 332 and alanine at position 236 (239D, 332E and 236A); aspartic acid at position 239, glutamic acid at position 332, alanine at position 236 and leucine at position 330 (239D, 332E, 236A and 330L); glutamic acid at position 267 (267E); tyrosine at position 234, tryptophan at position 236 and alanine at position 298 (234Y, 236W and 298A); glutamic acid at position 239 and serine at position 265 (239E and 265S); leucine at position 396 (396L); or leucine at position 243, proline at position 292 and leucine at position 300 (243 L, 292P and 300L). See, e.g., U.S. Pat. Nos. 7,662,925; 7,317,091; 8,093,357; 8,093,359; 8,388,955; 8,858,937; 8,937,158; 9,353,187; 10,184,000; 8,039,592; 8,383,109; 10,584,176; 8,735,545; 9,040,041; 8,735,545; 9,657,106; 9,296,815; 8,192,737; 8,652,466; 9,708,408; 10,711,069; 8,618,251; 9,657,101; and 8,742,074; U.S. Patent Publication Nos. 2018/355034 and 2021/214434 and Intl. Appl. No. WO 2019/125846, which are hereby incorporated herein by reference in their entireties for all purposes.

In some embodiments, the terminal Fc amino acid residue (e.g., K447 according to Eu numbering) is removed or eliminated from one or both of the first Fc domain and the second Fc domain.

In some embodiments, the first Fc domain and the second Fc domain comprise IgG4 amino acid sequences set forth, respectively, below, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, below: SEQ ID NOs: 888 and 889; SEQ ID NOs: 890 and 891; SEQ ID NOs: 892 and 891, SEQ ID NOs: 893 and 889; SEQ ID NOs: 888 and 894; SEQ ID NOs: 893 and 894; SEQ ID NOs: 895 and 891; SEQ ID NOs: 892 and 896; SEQ ID NOs: 895 and 896; SEQ ID NOs: 897 and 889; SEQ ID NOs: 888 and 898; SEQ ID NOs: 897 and 898; SEQ ID NOs: 899 and 891; SEQ ID NOs: 892 and 900; SEQ ID NOs: 899 and 900; or SEQ ID NOs: 890 and 891.

In some embodiments, the first Fc domain and the second Fc domain comprise IgG1 amino acid sequences set forth, respectively, below, or comprise amino acid sequences that are at least 80%, at least 85%, at least 90%, at least 91%, at least 92%, at least 93%, at least 94%, at least 95%, at least 96%, at least 97%, at least 98%, or at least 99% identical to the amino acid sequences set forth, respectively, below: SEQ ID NOs: 901 and 902; SEQ ID NOs: 903 and 904; SEQ ID NOs: 905 and 904; SEQ ID NOs: 906 and 902; SEQ ID NOs: 901 and 907; SEQ ID NOs: 908 and 909; SEQ ID NOs: 910 and 904; SEQ ID NOs: 905 and 911; SEQ ID NOs: 912 and 913; SEQ ID NOs: 914 and 902; SEQ ID NOs: 901 and 915; SEQ ID NOs: 914 and 915; SEQ ID NOs: 916 and 904; SEQ ID NOs: 905 and 917; SEQ ID NOs: 916 and 917; SEQ ID NOs: 918 and 904; or SEQ ID NOs: 919 and 920.

In various embodiments, the IL-15v or IL-15Rα Sushi domain-IL-15v fusion proteins described herein are directly fused to an Fc domain or are fused via a linker, e.g., a GS linker. Generally, the IL-15v or IL-15Rα Sushi domain-IL-15v fusion proteins described herein are fused to an Fc domain without or in the absence of a cleavable linker. The amino acid sequences of illustrative first and second Fc domains that can be incorporated into the herein described CD4-targeted IL-15v molecules are provided in Table F.

TABLE F
Fc regions - Illustrative heterodimeric pairs

| | | | | | |
|---|---|---|---|---|---|
| | SEQ | | | SEQ | |
| Fc aa | ID | | Fc aa | ID | |
| subst | NO: | First Fc region (Fab VH fusion) | subst | NO: | Second Fc region (IL-15-fusion) |

IgG4 variants

| | | | | | |
|---|---|---|---|---|---|
| S228P, | 888 | ESKYGPPCPPCPAPEAAGGPSVFLFPPKPKDTL | S228P, | 889 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| + | | EYKCKVSNKGLPSSIEKTISKAKGQPREPQVYT | T366S, | | PSSIEKTISKAKGQPREPQVYTLPPSQEEMTKNQVS |
| T366W | | LPPSQEEMTKNQVSLWCLVKGFYPSDIAVEWES | L368A, | | LSCAVKGFYPSDIAVEWESNGQPENNYKTTPPVLDS |
| | | NGQPENNYKTTPPVLDSDGSFFLYSRLTVDKSR | Y407V, | | DGSFFLVSRLTVDKSRWQEGNVFSCSVMHEALHNRF |
| | | WQEGNVFSCSVMHEALHNHYTQKSLSLSLG | H435R, | | TQKSLSLSLGK |
| | | | Y436F | | |
| S228P | 890 | ESKYGPPCPPCPAPEAAGGPSVFLFPPKPKDTL | F234A, | 891 | KYGPPCPPCPAPEAAGGPSVFLFPPKPKDTLMISRT |
| F234A, | | MISRTPEVTCVVVDVSQEDPEVQFNWYVDGVEV | L235A, | | PEVTCVVVDVSQEDPEVQFNWYVDGVEVHNAKTKPR |
| L235A, | | HNAKTKPREEQFNSTYRVVSVLTVLHQDWLNGK | T366W | | EEQFNSTYRVVSVLTVLHQDWLNGKEYKCKVSNKGL |
| T366S, | | EYKCKVSNKGLPSSIEKTISKAKGQPREPQVYT | | | PSSIEKTISKAKGQPREPQVYTLPPSQEEMTKNQVS |
| L368A, | | LPPSQEEMTKNQVSLSCAVKGFYPSDIAVEWES | | | LWCLVKGFYPSDIAVEWESNGQPENNYKTTPPVLDS |
| Y407V, | | NGQPENNYKTTPPVLDSDGSFFLVSRLTVDKSR | | | DGSFFLYSRLTVDKSRWQEGNVFSCSVMHEALHNHY |
| H435R | | WQEGNVFSCSVMHEALHNRYTQKSLSLSLGK | | | TQKSLSLSLGK |
| S228P, | 892 | ESKYGPPCPPCPAPEAAGGPSVFLFPPKPKDTL | S228P, | 891 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | T366W | | PSSIEKTISKAKGQPREPQVYTLPPSQEEMTKNQVS |
| L368A, | | LPPSQEEMTKNQVSLSCAVKGFYPSDIAVEWES | | | LWCLVKGFYPSDIAVEWESNGQPENNYKTTPPVLDS |
| Y407V, | | NGQPENNYKTTPPVLDSDGSFFLVSRLTVDKSR | | | DGSFFLYSRLTVDKSRWQEGNVFSCSVMHEALHNHY |
| H435R, | | WQEGNVFSCSVMHEALHNRFTQKSLSLSLG | | | TQKSLSLSLGK |
| Y436F | | | | | |
| S228P, | 893 | ESKYGPPCPPCPAPEAAGGPSVFLFPPKPKDTL | S228P, | 889 | KYGPPCPPCPAPEAAGGPSVFLFPPKPKDTLMISRT |
| F234A, | | YITREPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366W, | | EYKCKVSNKGLPSSIEKTISKAKGQPREPQVYT | T366S, | | PSSIEKTISKAKGQPREPQVYTLPPSQEEMTKNQVS |
| M252Y, | | LPPSQEEMTKNQVSLWCLVKGFYPSDIAVEWES | L368A, | | LSCAVKGFYPSDIAVEWESNGQPENNYKTTPPVLDS |
| S254T, | | NGQPENNYKTTPPVLDSDGSFFLYSRLTVDKSR | Y407V, | | DGSFFLVSRLTVDKSRWQEGNVFSCSVMHEALHNRF |
| T256E | | WQEGNVFSCSVMHEALHNHYTQKSLSLSLG | H435R, | | TQKSLSLSLGK |
| | | | Y436F | | |
| S228P, | 888 | ESKYGPPCPPCPAPEAAGGPSVFLFPPKPKDTL | S228P, | 894 | KYGPPCPPCPAPEAAGGPSVFLFPPKPKDTLYITRE |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366W | | EYKCKVSNKGLPSSIEKTISKAKGQPREPQVYT | M252Y, | | PSSIEKTISKAKGQPREPQVYTLPPSQEEMTKNQVS |
| | | LPPSQEEMTKNQVSLWCLVKGFYPSDIAVEWES | S254T, | | LSCAVKGFYPSDIAVEWESNGQPENNYKTTPPVLDS |
| | | NGQPENNYKTTPPVLDSDGSFFLYSRLTVDKSR | T256E, | | DGSFFLVSRLTVDKSRWQEGNVFSCSVMHEALHNRF |
| | | WQEGNVFSCSVMHEALHNHYTQKSLSLSLG | T366S, | | TQKSLSLSLGK |
| | | | L368A, | | |
| | | | Y407V, | | |
| | | | H435R, | | |
| | | | Y436F | | |
| S228P, | 893 | ESKYGPPCPPCPAPEAAGGPSVFLFPPKPKDTL | S228P, | 894 | KYGPPCPPCPAPEAAGGPSVFLFPPKPKDTLYITRE |
| F234A, | | YITREPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| M252Y, | | EYKCKVSNKGLPSSIEKTISKAKGQPREPQVYT | M252Y, | | PSSIEKTISKAKGQPREPQVYTLPPSQEEMTKNQVS |
| S254T, | | LPPSQEEMTKNQVSLWCLVKGFYPSDIAVEWES | S254T, | | LSCAVKGFYPSDIAVEWESNGQPENNYKTTPPVLDS |
| T256E, | | NGQPENNYKTTPPVLDSDGSFFLYSRLTVDKSR | T256E | | DGSFFLVSRLTVDKSRWQEGNVFSCSVMHEALHNRF |
| T366W | | WQEGNVFSCSVMHEALHNHYTQKSLSLSLG | T366S, | | TQKSLSLSLGK |
| | | | L368A, | | |
| | | | Y407V, | | |
| | | | H435R, | | |
| | | | Y436F | | |
| S228P, | 895 | ESKYGPPCPPCPAPEAAGGPSVFLFPPKPKDTL | S228P, | 891 | KYGPPCPPCPAPEAAGGPSVFLFPPKPKDTLMISRT |
| F234A, | | YITREPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| M252Y, | | EYKCKVSNKGLPSSIEKTISKAKGQPREPQVYT | T366W | | PSSIEKTISKAKGQPREPQVYTLPPSQEEMTKNQVS |
| S254T, | | LPPSQEEMTKNQVSLSCAVKGFYPSDIAVEWES | | | LWCLVKGFYPSDIAVEWESNGQPENNYKTTPPVLDS |
| T256E, | | NGQPENNYKTTPPVLDSDGSFFLVSRLTVDKSR | | | DGSFFLYSRLTVDKSRWQEGNVFSCSVMHEALHNHY |
| T366S, | | WQEGNVFSCSVMHEALHNRFTQKSLSLSLG | | | TQKSLSLSLGK |
| L368A, | | | | | |
| Y407V, | | | | | |
| H435R, | | | | | |
| Y436F, | | | | | |
| S228P, | 892 | ESKYGPPCPPCPAPEAAGGPSVFLFPPKPKDTL | S228P, | 896 | KYGPPCPPCPAPEAAGGPSVFLFPPKPKDTLYITRE |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | M252Y, | | PSSIEKTISKAKGQPREPQVYTLPPSQEEMTKNQVS |
| L368A, | | LPPSQEEMTKNQVSLSCAVKGFYPSDIAVEWES | S254T, | | LWCLVKGFYPSDIAVEWESNGQPENNYKTTPPVLDS |
| Y407V, | | NGQPENNYKTTPPVLDSDGSFFLVSRLTVDKSR | T256E, | | DGSFFLYSRLTVDKSRWQEGNVFSCSVMHEALHNHY |
| H435R, | | WQEGNVFSCSVMHEALHNRFTQKSLSLSLG | T366W | | TQKSLSLSLGK |
| Y436F | | | | | |
| S228P, | 895 | ESKYGPPCPPCPAPEAAGGPSVFLFPPKPKDTL | S228P, | 896 | KYGPPCPPCPAPEAAGGPSVFLFPPKPKDTLYITRE |
| F234A, | | YITREPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| M252Y, | | EYKCKVSNKGLPSSIEKTISKAKGQPREPQVYT | M252Y, | | PSSIEKTISKAKGQPREPQVYTLPPSQEEMTKNQVS |
| S254T, | | LPPSQEEMTKNQVSLSCAVKGFYPSDIAVEWES | S254T, | | LWCLVKGFYPSDIAVEWESNGQPENNYKTTPPVLDS |
| T256E, | | NGQPENNYKTTPPVLDSDGSFFLVSRLTVDKSR | T256E, | | DGSFFLYSRLTVDKSRWQEGNVFSCSVMHEALHNHY |
| T366S, | | WQEGNVFSCSVMHEALHNRFTQKSLSLSLG | T366W | | TQKSLSLSLGK |
| L368A, | | | | | |
| Y407V, | | | | | |
| H435R, | | | | | |
| Y436F | | | | | |
| S228P, | 897 | ESKYGPPCPPCPAPEAAGGPSVFLFPPKPKDTL | S228P, | 889 | KYGPPCPPCPAPEAAGGPSVELFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366W, | | EYKCKVSNKGLPSSIEKTISKAKGQPREPQVYT | T366S, | | PSSIEKTISKAKGQPREPQVYTLPPSQEEMTKNQVS |
| M428L, | | LPPSQEEMTKNQVSLWCLVKGFYPSDIAVEWES | L368A, | | LSCAVKGFYPSDIAVEWESNGQPENNYKTTPPVLDS |
| N434S | | NGQPENNYKTTPPVLDSDGSFFLYSRLTVDKSR | Y407V, | | DGSFFLVSRLTVDKSRWQEGNVFSCSVMHEALHNRF |
| | | WQEGNVFSCSVLHEALHSHYTQKSLSLSLG | H435R, | | TQKSLSLSLGK |
| | | | Y436F, | | |
| S228P, | 888 | ESKYGPPCPPCPAPEAAGGPSVFLFPPKPKDTL | S228P, | 898 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366W | | EYKCKVSNKGLPSSIEKTISKAKGQPREPQVYT | T366S, | | PSSIEKTISKAKGQPREPQVYTLPPSQEEMTKNQVS |
| | | LPPSQEEMTKNQVSLWCLVKGFYPSDIAVEWES | L368A, | | LSCAVKGFYPSDIAVEWESNGQPENNYKTTPPVLDS |
| | | NGQPENNYKTTPPVLDSDGSFFLYSRLTVDKSR | Y407V, | | DGSFFLVSRLTVDKSRWQEGNVFSCSVLHEALHSRF |
| | | WQEGNVFSCSVMHEALHNHYTQKSLSLSLG | M428L, | | TQKSLSLSLGK |
| | | | N434S | | |
| S228P, | 897 | ESKYGPPCPPCPAPEAAGGPSVFLFPPKPKDTL | S228P, | 898 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366W, | | EYKCKVSNKGLPSSIEKTISKAKGQPREPQVYT | T366S, | | PSSIEKTISKAKGQPREPQVYTLPPSQEEMTKNQVS |
| M428L, | | LPPSQEEMTKNQVSLWCLVKGFYPSDIAVEWES | L368A, | | LSCAVKGFYPSDIAVEWESNGQPENNYKTTPPVLDS |
| N434S | | NGQPENNYKTTPPVLDSDGSFFLYSRLTVDKSR | Y407V, | | DGSFFLVSRLTVDKSRWQEGNVFSCSVLHEALHSRF |
| | | WQEGNVFSCSVLHEALHSHYTQKSLSLSLG | M428L, | | TQKSLSLSLGK |
| | | | N434S | | |
| S228P, | 899 | ESKYGPPCPPCPAPEAAGGPSVFLFPPKPKDTL | S228P, | 891 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | T366W | | PSSIEKTISKAKGQPREPQVYTLPPSQEEMTKNQVS |
| L368A, | | LPPSQEEMTKNQVSLSCAVKGFYPSDIAVEWES | | | LWCLVKGFYPSDIAVEWESNGQPENNYKTTPPVLDS |
| Y407V, | | NGQPENNYKTTPPVLDSDGSFFLVSRLTVDKSR | | | DGSFFLYSRLTVDKSRWQEGNVFSCSVMHEALHNHY |
| M428L, | | WQEGNVFSCSVLHEALHSRFTQKSLSLSLG | | | TQKSLSLSLGK |
| N434S, | | | | | |
| H435R, | | | | | |
| Y436F, | | | | | |
| S228P, | 892 | ESKYGPPCPPCPAPEAAGGPSVFLFPPKPKDTL | S228P, | 900 | KYGPPCPPCPAPEAAGGPSVELFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | T366W, | | PSSIEKTISKAKGQPREPQVYTLPPSQEEMTKNQVS |
| L368A, | | LPPSQEEMTKNQVSLSCAVKGFYPSDIAVEWES | M428L, | | LWCLVKGFYPSDIAVEWESNGQPENNYKTTPPVLDS |
| Y407V, | | NGQPENNYKTTPPVLDSDGSFFLVSRLTVDKSR | N434S | | DGSFFLYSRLTVDKSRWQEGNVESCSVLHEALHSHY |
| H435R, | | WQEGNVFSCSVMHEALHNRFTQKSLSLSLG | | | TQKSLSLSLGK |
| Y436F | | | | | |
| S228P, | 899 | ESKYGPPCPPCPAPEAAGGPSVFLFPPKPKDTL | S228P, | 900 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | T366W, | | PSSIEKTISKAKGQPREPQVYTLPPSQEEMTKNQVS |
| L368A, | | LPPSQEEMTKNQVSLSCAVKGFYPSDIAVEWES | M428L, | | LWCLVKGFYPSDIAVEWESNGQPENNYKTTPPVLDS |
| Y407V, | | NGQPENNYKTTPPVLDSDGSFFLVSRLTVDKSR | N434S | | DGSFFLYSRLTVDKSRWQEGNVFSCSVLHEALHSHY |
| M428L, | | WQEGNVFSCSVLHEALHSRFTQKSLSLSLG | | | TQKSLSLSLGK |
| N434S, | | | | | |
| H435R, | | | | | |
| Y436F | | | | | |
| S228P, | 890 | ESKYGPPCPPCPAPEAAGGPSVFLFPPKPKDTL | S228P, | 891 | KYGPPCPPCPAPEAAGGPSVFLFPPKPKDTLMISRT |
| F234A, | | MISRTPEVTCVVVDVSQEDPEVQFNWYVDGVEV | F234A, | | PEVTCVVVDVSQEDPEVQFNWYVDGVEVHNAKTKPR |
| L235A, | | HNAKTKPREEQFNSTYRVVSVLTVLHQDWLNGK | L235A, | | EEQFNSTYRVVSVLTVLHQDWLNGKEYKCKVSNKGL |
| T366S, | | EYKCKVSNKGLPSSIEKTISKAKGQPREPQVYT | T366W | | PSSIEKTISKAKGQPREPQVYTLPPSQEEMTKNQVS |
| L368A, | | LPPSQEEMTKNQVSLSCAVKGFYPSDIAVEWES | | | LWCLVKGFYPSDIAVEWESNGQPENNYKTTPPVLDS |
| Y407V, | | NGQPENNYKTTPPVLDSDGSFFLVSRLTVDKSR | | | DGSFFLYSRLTVDKSRWQEGNVFSCSVMHEALHNHY |
| H435R | | WQEGNVFSCSVMHEALHNRYTQKSLSLSLGK | | | TQKSLSLSLGK |

IgG1 variants

| | | | | | |
|---|---|---|---|---|---|
| L234A, | 901 | EPKSSDKTHTCPPCPAPEAAGGPSVFLFPPKPK | L234A, | 902 | DKTHTCPPCPAPEAAGGPSVFLFPPKPKDTLMISRT |
| L235A, | | DTLMISRTPEVTCVVVDVSHEDPEVKFNWYVDG | L235A, | | PEVTCVVVDVSHEDPEVKENWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366W | | NGKEYKCKVSNKALPASIEKTISKAKGQPREPQ | T366S, | | PASIEKTISKAKGQPREPQVYTLPPSREEMTKNQVS |
| | | VYTLPPSREEMTKNQVSLWCLVKGFYPSDIAVE | L368A, | | LSCAVKGFYPSDIAVEWESNGQPENNYKTTPPVLDS |
| | | WESNGQPENNYKTTPPVLDSDGSFFLYSKLTVD | Y407V, | | DGSFFLVSKLTVDKSRWQQGNVFSCSVMHEALHNRF |
| | | KSRWQQGNVFSCSVMHEALHNHYTQKSLSLSPG | H435R, | | TQKSLSLSPGK |
| | | | Y436F | | |
| L234A, | 903 | EPKSCDKTHTCPPCPAPEAAGGPSVFLFPPKPK | L234A, | 904 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | T366W | | PASIEKTISKAKGQPREPQVYTLPPSREEMTKNQVS |
| L368A, | | VYTLPPSREEMTKNQVSLSCAVKGFYPSDIAVE | | | LWCLVKGFYPSDIAVEWESNGQPENNYKTTPPVLDS |
| Y407V, | | WESNGQPENNYKTTPPVLDSDGSFFLVSKLTVD | | | DGSFFLYSKLTVDKSRWQQGNVFSCSVMHEALHNHY |
| H435R | | KSRWQQGNVFSCSVMHEALHNRYTQKSLSLSPG | | | TQKSLSLSPGK |
| L234A, | 905 | EPKSCDKTHTCPPCPAPEAAGGPSVFLFPPKPK | L234A, | 904 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | T366W | | PASIEKTISKAKGQPREPQVYTLPPSREEMTKNQVS |
| L368A, | | VYTLPPSREEMTKNQVSLSCAVKGFYPSDIAVE | | | LWCLVKGFYPSDIAVEWESNGQPENNYKTTPPVLDS |
| Y407V, | | WESNGQPENNYKTTPPVLDSDGSFFLVSKLTVD | | | DGSFFLYSKLTVDKSRWQQGNVFSCSVMHEALHNHY |
| H435R, | | KSRWQQGNVFSCSVMHEALHNRFTQKSLSLSPG | | | TQKSLSLSPGK |
| Y436F | | | | | |
| L234A, | 906 | EPKSSDKTHTCPPCPAPEAAGGPSVFLFPPKPK | L234A, | 902 | DKTHTCPPCPAPEAAGGPSVFLFPPKPKDTLMISRT |
| L235A, | | DTLYITREPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLIVLHQDWL | P331S, | | EEQYNSTYRVVSVLTVLHQDWLNGKEYKCKVSNKAL |
| M252Y, | | NGKEYKCKVSNKALPASIEKTISKAKGQPREPQ | T366S, | | PASIEKTISKAKGQPREPQVYTLPPSREEMTKNQVS |
| S254T, | | VYTLPPSREEMTKNQVSLWCLVKGFYPSDIAVE | L368A, | | LSCAVKGFYPSDIAVEWESNGQPENNYKTTPPVLDS |
| T256E, | | WESNGQPENNYKTTPPVLDSDGSFFLYSKLTVD | Y407V, | | DGSFFLVSKLTVDKSRWQQGNVFSCSVMHEALHNRE |
| T366W | | KSRWQQGNVFSCSVMHEALHNHYTQKSLSLSPG | H435R, | | TQKSLSLSPGK |
| | | | Y436F | | |
| L234A, | 901 | EPKSSDKTHTCPPCPAPEAAGGPSVFLFPPKPK | L234A, | 907 | DKTHTCPPCPAPEAAGGPSVFLFPPKPKDTLYITRE |
| L235A, | | DTLMISRTPEVTCVVVDVSHEDPEVKENWYVDG | L235A, | | PEVTCVVVDVSHEDPEVKENWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366W | | NGKEYKCKVSNKALPASIEKTISKAKGQPREPQ | M252Y | | PASIEKTISKAKGQPREPQVYTLPPSREEMTKNQVS |
| | | VYTLPPSREEMTKNQVSLWCLVKGFYPSDIAVE | S254T, | | LSCAVKGFYPSDIAVEWESNGQPENNYKTTPPVLDS |
| | | WESNGQPENNYKTTPPVLDSDGSFFLYSKLTVD | T256E, | | DGSFFLVSKLTVDKSRWQQGNVFSCSVMHEALHNRF |
| | | KSRWQQGNVFSCSVMHEALHNHYTQKSLSLSPG | T366S, | | TQKSLSLSPGK |
| | | | L368A, | | |
| | | | Y407V, | | |
| | | | H435R, | | |
| | | | Y436F | | |
| L234A, | 908 | EPKSCDKTHTCPPCPAPEAAGGPSVFLFPPKPK | L234A, | 909 | DKTHTCPPCPAPEAAGGPSVFLFPPKPKDTLYITRE |
| L235A, | | DTLYITREPEVTCVVVDVSHEDPEVKFNWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| M252Y | | NGKEYKCKVSNKALPASIEKTISKAKGQPREPQ | M252Y, | | PASIEKTISKAKGQPREPQVYTLPPSREEMTKNQVS |
| S254T, | | VYTLPPSREEMTKNQVSLSCAVKGFYPSDIAVE | S254T, | | LWCLVKGFYPSDIAVEWESNGQPENNYKTTPPVLDS |
| T256E, | | WESNGQPENNYKTTPPVLDSDGSFFLVSKLTVD | T256E, | | DGSFFLYSKLTVDKSRWQQGNVFSCSVMHEALHNRF |
| T366W | | KSRWQQGNVFSCSVMHEALHNHYTQKSLSLSPG | T366S, | | TQKSLSLSPGK |
| | | | L368A, | | |
| | | | Y407V, | | |
| | | | H435R, | | |
| | | | Y436F | | |
| L234A, | 910 | EPKSCDKTHTCPPCPAPEAAGGPSVELFPPKPK | L234A, | 904 | DKTHTCPPCPAPEAAGGPSVFLFPPKPKDTLMISRT |
| L235A, | | DTLYITREPEVTCVVVDVSHEDPEVKENWYVDG | L235A, | | PEVTCVVVDVSHEDPEVKENWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| M252Y, | | NGKEYKCKVSNKALPASIEKTISKAKGQPREPQ | T366W | | PASIEKTISKAKGQPREPQVYTLPPSREEMTKNQVS |
| S254T, | | VYTLPPSREEMTKNQVSLSCAVKGFYPSDIAVE | | | LWCLVKGFYPSDIAVEWESNGQPENNYKTTPPVLDS |
| T256E, | | WESNGQPENNYKTTPPVLDSDGSFFLVSKLTVD | | | DGSFFLYSKLTVDKSRWQQGNVFSCSVMHEALHNHY |
| T366S, | | KSRWQQGNVFSCSVMHEALHNRFTQKSLSLSPG | | | TQKSLSLSPGK |
| L368A, | | | | | |
| Y407V | | | | | |
| L234A, | 905 | EPKSCDKTHTCPPCPAPEAAGGPSVFLFPPKPK | L234A, | 911 | DKTHTCPPCPAPEAAGGPSVFLFPPKPKDTLYITRE |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | M252Y, | | PASIEKTISKAKGQPREPQVYTLPPSREEMTKNQVS |
| L368A, | | VYTLPPSREEMTKNQVSLSCAVKGFYPSDIAVE | S254T, | | LWCLVKGFYPSDIAVEWESNGQPENNYKTTPPVLDS |
| Y407V, | | WESNGQPENNYKTTPPVLDSDGSFFLVSKLTVD | T256E, | | DGSFFLYSKLTVDKSRWQQGNVFSCSVMHEALHNHY |
| H435R, | | KSRWQQGNVFSCSVMHEALHNRFTQKSLSLSPG | T366W, | | TQKSLSLSPGK |
| Y436F | | | | | |
| L234A, | 912 | EPKSSDKTHTCPPCPAPEAAGGPSVFLFPPKPK | L234A, | 913 | DKTHTCPPCPAPEAAGGPSVFLFPPKPKDTLYITRE |
| L235A, | | DTLYITREPEVTCVVVDVSHEDPEVKFNWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| M252Y | | NGKEYKCKVSNKALPASIEKTISKAKGQPREPQ | M252Y, | | PASIEKTISKAKGQPREPQVYTLPPSREEMTKNQVS |
| S254T, | | VYTLPPSREEMTKNQVSLWCLVKGFYPSDIAVE | S254T, | | LSCAVKGFYPSDIAVEWESNGQPENNYKTTPPVLDS |
| T256E, | | WESNGQPENNYKTTPPVLDSDGSFFLYSKLTVD | T256E, | | DGSFFLVSKLTVDKSRWQQGNVFSCSVMHEALHNHY |
| T366S, | | KSRWQQGNVFSCSVMHEALHNRFTQKSLSLSPG | T366W | | TQKSLSLSPGK |
| L368A, | | | | | |
| Y407V, | | | | | |
| H435R, | | | | | |
| Y436F | | | | | |
| L234A, | 914 | EPKSSDKTHTCPPCPAPEAAGGPSVFLFPPKPK | L234A, | 902 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366W, | | NGKEYKCKVSNKALPASIEKTISKAKGQPREPQ | T366S, | | PASIEKTISKAKGQPREPQVYTLPPSREEMTKNQVS |
| M428L, | | VYTLPPSREEMTKNQVSLWCLVKGFYPSDIAVE | L368A, | | LSCAVKGFYPSDIAVEWESNGQPENNYKTTPPVLDS |
| N434S | | WESNGQPENNYKTTPPVLDSDGSFFLYSKLTVD | Y407V, | | DGSFFLVSKLTVDKSRWQQGNVFSCSVMHEALHNRF |
| | | KSRWQQGNVFSCSVLHEALHSHYTQKSLSLSPG | H435R, | | TQKSLSLSPGK |
| | | | Y436F, | | |
| L234A, | 901 | EPKSSDKTHTCPPCPAPEAAGGPSVFLFPPKPK | L234A, | 915 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKENWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366W | | NGKEYKCKVSNKALPASIEKTISKAKGQPREPQ | T366S, | | PASIEKTISKAKGQPREPQVYTLPPSREEMTKNQVS |
| | | VYTLPPSREEMTKNQVSLWCLVKGFYPSDIAVE | L368A, | | LSCAVKGFYPSDIAVEWESNGQPENNYKTTPPVLDS |
| | | WESNGQPENNYKTTPPVLDSDGSFFLYSKLTVD | Y407V, | | DGSFFLVSKLTVDKSRWQQGNVFSCSVLHEALHSRF |
| | | KSRWQQGNVFSCSVMHEALHNHYTQKSLSLSPG | M428L, | | TQKSLSLSPGK |
| | | | N434S, | | |
| | | | H435R | | |
| | | | Y436F | | |
| L234A, | 914 | EPKSSDKTHTCPPCPAPEAAGGPSVFLFPPKPK | L234A, | 915 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKENWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366W, | | NGKEYKCKVSNKALPASIEKTISKAKGQPREPQ | T366S, | | PASIEKTISKAKGQPREPQVYTLPPSREEMTKNQVS |
| M428L, | | VYTLPPSREEMTKNQVSLWCLVKGFYPSDIAVE | L368A, | | LSCAVKGFYPSDIAVEWESNGQPENNYKTTPPVLDS |
| N434S | | WESNGQPENNYKTTPPVLDSDGSFFLYSKLTVD | Y407V, | | DGSFFLVSKLTVDKSRWQQGNVFSCSVLHEALHSRF |
| | | KSRWQQGNVFSCSVLHEALHSHYTQKSLSLSPG | M428L, | | TQKSLSLSPGK |
| | | | N434S, | | |
| | | | H435R, | | |
| | | | Y436F | | |
| L234A, | 916 | EPKSCDKTHTCPPCPAPEAAGGPSVFLFPPKPK | L234A, | 904 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | T366W | | PASIEKTISKAKGQPREPQVYTLPPSREEMTKNQVS |
| L368A, | | VYTLPPSREEMTKNQVSLSCAVKGFYPSDIAVE | | | LWCLVKGFYPSDIAVEWESNGQPENNYKTTPPVLDS |
| Y407V, | | WESNGQPENNYKTTPPVLDSDGSFFLVSKLTVD | | | DGSFFLYSKLTVDKSRWQQGNVFSCSVMHEALHNHY |
| M428L, | | KSRWQQGNVFSCSVLHEALHSRFTQKSLSLSPG | | | TQKSLSLSPGK |
| N434S, | | | | | |
| H435R, | | | | | |
| Y436F | | | | | |
| L234A, | 905 | EPKSCDKTHTCPPCPAPEAAGGPSVFLFPPKPK | L234A, | 917 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | T366W, | | PASIEKTISKAKGQPREPQVYTLPPSREEMTKNQVS |
| L368A, | | VYTLPPSREEMTKNQVSLSCAVKGFYPSDIAVE | M428L, | | LWCLVKGFYPSDIAVEWESNGQPENNYKTTPPVLDS |
| Y407V, | | WESNGQPENNYKTTPPVLDSDGSFFLVSKLTVD | N434S | | DGSFFLYSKLTVDKSRWQQGNVFSCSVLHEALHSHY |
| H435R, | | KSRWQQGNVFSCSVMHEALHNRFTQKSLSLSPG | | | TQKSLSLSPGK |
| Y436F | | | | | |
| L234A, | 916 | EPKSCDKTHTCPPCPAPEAAGGPSVFLFPPKPK | L234A, | 917 | DKTHTCPPCPAPEAAGGPSVFLFPPKPKDTLMISRT |
| L235A, | | DTLMISRTPEVTCVVVDVSHEDPEVKENWYVDG | L235A, | | PEVTCVVVDVSHEDPEVKFNWYVDGVEVHNAKTKPR |
| P331S, | | VEVHNAKTKPREEQYNSTYRVVSVLTVLHQDWL | P331S, | | EEQYNSTYRVVSVLTVLHQDWLNGKEYKCKVSNKAL |
| T366S, | | NGKEYKCKVSNKALPASIEKTISKAKGQPREPQ | T366W, | | PASIEKTISKAKGQPREPQVYTLPPSREEMTKNQVS |
| L368A, | | VYTLPPSREEMTKNQVSLSCAVKGFYPSDIAVE | M428L, | | LWCLVKGFYPSDIAVEWESNGQPENNYKTTPPVLDS |
| Y407V, | | WESNGQPENNYKTTPPVLDSDGSFFLVSKLTVD | N434S | | DGSFFLYSKLTVDKSRWQQGNVFSCSVLHEALHSHY |
| M428L, | | KSRWQQGNVFSCSVLHEALHSRFTQKSLSLSPG | | | TQKSLSLSPGK |
| N434S, | | | | | |
| H435R, | | | | | |
| Y436F | | | | | |
| L234A, | 918 | DKTHTCPPCPAPEAAGGPSVFLFPPKPKDTLMI | L234A, | 904 | DKTHTCPPCPAPEAAGGPSVFLFPPKPKDTLMISRT |
| L235A, | | SRTPEVTCVVVDVSHEDPEVKFNWYVDGVEVHN | L235A, | | PEVTCVVVDVSHEDPEVKENWYVDGVEVHNAKTKPR |
| P331S, | | AKTKPREEQYNSTYRVVSVLTVLHQDWLNGKEY | P331S, | | EEQYNSTYRVVSVLTVLHQDWLNGKEYKCKVSNKAL |
| T366S, | | KCKVSNKALPASIEKTISKAKGQPREPQVYTLP | T366W | | PASIEKTISKAKGQPREPQVYTLPPSREEMTKNQVS |
| L368A, | | PSREEMTKNQVSLSCAVKGFYPSDIAVEWESNG | | | LWCLVKGFYPSDIAVEWESNGQPENNYKTTPPVLDS |
| Y407V, | | QPENNYKTTPPVLDSDGSFFLVSKLTVDKSRWQ | | | DGSFFLYSKLTVDKSRWQQGNVFSCSVMHEALHNHY |
| H435R | | QGNVFSCSVMHEALHNRYTQKSLSLSPGK | | | TQKSLSLSPGK |
| L234A, | 919 | DKTHTCPPCPAPEAAGAPSVFLFPPKPKDTLMI | L234A, | 920 | DKTHTCPPCPAPEAAGAPSVFLFPPKPKDTLMISRT |
| L235A, | | SRTPEVTCVVVDVSHEDPEVKFNWYVDGVEVHN | L235A, | | PEVTCVVVDVSHEDPEVKFNWYVDGVEVHNAKTKPR |
| G237A, | | AKTKPREEQYNSTYRVVSVLTVLHQDWLNGKEY | G237A, | | EEQYNSTYRVVSVLTVLHQDWLNGKEYKCKVSNKAL |
| T366S, | | KCKVSNKALPAPIEKTISKAKGQPREPQVYTLP | T366W | | PAPIEKTISKAKGQPREPQVYTLPPSREEMTKNQVS |
| L368A, | | PSREEMTKNQVSLSCAVKGFYPSDIAVEWESNG | | | LWCLVKGFYPSDIAVEWESNGQPENNYKTTPPVLDS |
| Y407V, | | QPENNYKTTPPVLDSDGSFFLVSKLTVDKSRWQ | | | DGSFFLYSKLTVDKSRWQQGNVFSCSVMHEALHNHY |
| H435R | | QGNVFSCSVMHEALHNRYTQKSLSLSPGK | | | TQKSLSLSPGK |

e. CD4-Targeted IL-15v Molecules

The amino acid sequences of illustrative CD4-targeted IL-15v molecules described herein, and suitable for administration to a human, are provided in Table G. The amino acid sequences of illustrative simianized CD4-targeted IL-15v molecules described herein, and suitable for administration to a non-human primate, are provided in Table H.

### Three Polypeptide Format—Binds to CD4 D1

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 921, 922 and 923 (100); SEQ ID NOs: 924, 922 and 925 (101); SEQ ID NOs: 926, 922 and 925 (102); SEQ ID NOs: 926, 922 and 1074 (103); SEQ ID NOs: 926, 922 and 1075 (104); SEQ ID NOs: 926, 922 and 1076 (105); SEQ ID NOs: 926, 922 and 974 (106); SEQ ID NOs: 926, 929 and 1076 (107); SEQ ID NOs: 927, 928 and 925 (108); SEQ ID NOs: 926, 929 and 925 (109); SEQ ID NOs: 926, 922 and 985 (110); SEQ ID NOs: 930, 922 and 923 (111); SEQ ID NOs: 931, 922 and 925 (112); SEQ ID NOs: 932, 922 and 933 (113); SEQ ID NOs: 934, 922 and 935 (114); SEQ ID NOs: 936, 937 and 923 (115); SEQ ID NOs: 938, 939 and 923 (116); SEQ ID NOs: 940, 941 and 923 (117); SEQ ID NOs: 942, 943 and 923 (118); SEQ ID NOs: 944, 945 and 923 (119); SEQ ID NOs: 946, 947 and 923 (120); SEQ ID NOs: 948, 949 and 923 (121); SEQ ID NOs: 950, 951 and 923 (122); SEQ ID NOs: 952, 953 and 923 (123); SEQ ID NOs: 954, 955 and 923 (124); SEQ ID NOs: 956, 955 and 923 (125); SEQ ID NOs: 957, 958 and 923 (126); SEQ ID NOs: 959, 960 and 923 (127); SEQ ID NOs: 961, 962 and 923 (128); SEQ ID NOs: 961, 960 and 923 (129); SEQ ID NOs: 926, 922 and 1091 (249); SEQ ID NOs: 926, 922 and 1092 (251); SEQ ID NOs: 926, 922 and 1093 (252); SEQ ID NOs: 926, 922 and 1094 (253); SEQ ID NOs: 926, 922 and 1095 (254); SEQ ID NOs: 926, 922 and 1096 (255); SEQ ID NOs: 926, 922 and 1097 (259); SEQ ID NOs: 926, 922 and 1098 (260); SEQ ID NOs: 926, 922 and 1099 (261) or SEQ ID NOs: 926, 922 and 1100 (262).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 921, 922 and 923 (100).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 924, 922 and 925 (101).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 925 (102).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1074 (103).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1075 (104).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1076 (105).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 974 (106).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 929 and 1076 (107).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 927, 928 and 925 (108).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 929 and 925 (109).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 985 (110).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 930, 922 and 923 (111).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 931, 922 and 925 (112).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 932, 922 and 933 (113).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 934, 922 and 935 (114).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 936, 937 and 923 (115).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 938, 939 and 923 (116).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 940, 941 and 923 (117).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 942, 943 and 923 (118).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 944, 945 and 923 (119).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 946, 947 and 923 (120).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 948, 949 and 923 (121).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 950, 951 and 923 (122).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 952, 953 and 923 (123).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 954, 955 and 923 (124).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 956, 955 and 923 (125).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 957, 958 and 923 (126).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 959, 960 and 923 (127).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 961, 962 and 923 (128).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 961, 960 and 923 (129).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1091 (249).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1092 (251).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1093 (252).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1094 (253).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1095 (254).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1096 (255).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1097 (259).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1098 (260).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1099 (261).

In some embodiments, the CD4-targeted or CD4-binding IL-15v molecule comprises a heavy chain (HC) and a light chain (LC) that bind to CD4 D1 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 926, 922 and 1100 (262).

### Three Polypeptide Format-Binds to CD4 D2

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 1086, 1087 and 1088 (245); SEQ ID NOs: 963, 964 and 923 (130); SEQ ID NOs: 965, 966 and 923 (131); SEQ ID NOs: 967, 968 and 923 (132); SEQ ID NOs: 969, 970 and 923 (133); SEQ ID NOs: 969, 970 and 971 (134); SEQ ID NOs: 969, 970 and 972 (135); SEQ ID NOs: 973, 970 and 974 (136); SEQ ID NOs: 975, 976 and 923 (137); SEQ ID NOs: 977, 978 and 923 (138); SEQ ID NOs: 977, 978 and 971 (139); SEQ ID NOs: 977, 978 and 972 (140); SEQ ID NOs: 977, 979 and 972; (141); SEQ ID NOs: 980, 979 and 974 (142); SEQ ID NOs: 981, 982 and 923 (143); SEQ ID NOs: 981, 982 and 971 (144); SEQ ID NOs: 981, 982 and 972 (145); SEQ ID NOs: 981, 983 and 972 (146); SEQ ID NOs: 984, 983 and 974 (147); SEQ ID NOs: 984, 983 and 985 (148); SEQ ID NOs: 986, 987 and 923 (149); SEQ ID NOs: 988, 989 and 923 (150); SEQ ID NOs: 988, 989 and 971 (151); SEQ ID NOs: 988, 989 and 972 (152); SEQ ID NOs: 990, 991 and 923 (153); SEQ ID NOs: 990, 991 and 971 (154); SEQ ID NOs: 990, 991 and 972 (155) or SEQ ID NOs: 992, 991 and 974 (156).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1086, 1087 and 1088 (245).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 963, 964 and 923 (130).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 965, 966 and 923 (131).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 967, 968 and 923 (132).

In some embodiments, the CD4-targeted or CD4-binding IL-15v molecule comprises a heavy chain (HC) and a light chain (LC) that bind to CD4 D2 and an IL15RA Sushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 969, 970 and 923 (133).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 969, 970 and 971 (134).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 969, 970 and 972 (135).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 973, 970 and 974 (136).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 975, 976 and 923 (137).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 977, 978 and 923 (138).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 977, 978 and 971 (139).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 977, 978 and 972 (140).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 977, 979 and 972 (141).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 980, 979 and 974 (142).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 981, 982 and 923 (143).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 981, 982 and 971 (144).

In some embodiments, the CD4-targeted or CD4-binding IL-15v molecule comprises a heavy chain (HC) and a light chain (LC) that bind to CD4 D2 and an IL15RA Sushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 981, 982 and 972 (145).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 981, 983 and 972 (146).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 984, 983 and 974 (147).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 984, 983 and 985 (148).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 986, 987 and 923 (149).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 988, 989 and 923 (150).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 988, 989 and 971 (151).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 988, 989 and 972 (152).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 990, 991 and 923 (153).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 990, 991 and 971 (154).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 990, 991 and 972 (155).

In some embodiments, the CD4-targeted or CD4-binding IL-15v molecule comprises a heavy chain (HC) and a light chain (LC) that bind to CD4 D2 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 992, 991 and 974 (156).

### Three Polypeptide Format-Binds to One or Both of CD4 D2 and CD4 D3

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 993, 994 and 923 (157); SEQ ID NOs: 995, 996 and 923 (158); SEQ ID NOs: 997, 998 and 923 (159); SEQ ID NOs: 999, 1000 and 923 (160); SEQ ID NOs: 1001, 1002 and 923 (161); SEQ ID NOs: 1003, 1004 and 923 (162); SEQ ID NOs: 1005, 1006 and 923 (163) or SEQ ID NOs: 1007, 1008 and 923 (164).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 993, 994 and 923 (157).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 995, 996 and 923 (158).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 997, 998 and 923 (159).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 999, 1000 and 923 (160).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1001, 1002 and 923 (161).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1003, 1004 and 923 (162).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1005, 1006 and 923 (163).

In some embodiments, the CD4-targeted or CD4-binding IL-15v molecule comprises a heavy chain (HC) and a light chain (LC) that bind to one or both of CD4 D2 and CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1007, 1008 and 923 (164).

### Three Polypeptide Format-Binds to CD4 D3

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 1089, 1090 and 1088 (246); SEQ ID NOs: 1009, 1010 and 923 (165); SEQ ID NOs: 1009, 1010 and 971 (166); SEQ ID NOs: 1009, 1010 and 972 (167); SEQ ID NOs: 1011, 1012 and 923 (168); SEQ ID NOs: 1011, 1012 and 971 (169); SEQ ID NOs: 1011, 1012 and 972 (170); SEQ ID NOs: 1013, 1012 and 974 (171); SEQ ID NOs: 1011, 1014 and 923 (172); SEQ ID NOs: 1011, 1014 and 971 (173); SEQ ID NOs: 1011, 1014 and 972 (174); SEQ ID NOs: 1013, 1014 and 974 (175); SEQ ID NOs: 1015, 1016 and 923 (176); SEQ ID NOs: 1015, 1016 and 971 (177); SEQ ID NOs: 1015, 1017 and 971 (178); SEQ ID NOs: 1015, 1016 and 972 (179); SEQ ID NOs: 1015, 1017 and 972 (180); SEQ ID NOs: 1018, 1017 and 974 (181); SEQ ID NOs: 1018, 1017 and 985 (182); SEQ ID NOs: 1019, 1020 and 923 (183); SEQ ID NOs: 1019, 1020 and 971 (184); SEQ ID NOs: 1019, 1020 and 972 (185); SEQ ID NOs: 1021, 1020 and 974 (186); SEQ ID NOs: 1022, 1023 and 923 (187); SEQ ID NOs: 1024, 1025 and 923 (188); SEQ ID NOs: 1011, 1014 and 1026 (189); SEQ ID NOs: 1027, 1014 and 1028 (190); SEQ ID NOs: 1027, 1014 and 1029 (191); SEQ ID NOs: 1011, 1014 and 1030 (192); SEQ ID NOs: 1011, 1014 and 1031 (193); SEQ ID NOs: 1011, 1014 and 1032 (194); SEQ ID NOs: 1011, 1014 and 1077 (220); SEQ ID NOs: 1011, 1014 and 1033 (195); SEQ ID NOs: 1011, 1014 and 1034 (196); SEQ ID NOs: 1011, 1014 and 1035 (197); SEQ ID NOs: 1011, 1014 and 1036 (198); SEQ ID NOs: 1011, 1014 and 1037 (199); SEQ ID NOs: 1011, 1014 and 1038 (200); SEQ ID NOs: 1011, 1014 and 1039 (201); SEQ ID NOs: 1011, 1014 and 1040 (202); SEQ ID NOs: 1011, 1014 and 1041 (203); SEQ ID NOs: 1011, 1014 and 1042 (204); SEQ ID NOs: 1011, 1014 and 1043 (205); SEQ ID NOs: 1011, 1014 and 1044 (206); SEQ ID NOs: 1011, 1014 and 1045 (207); SEQ ID NOs: 1011, 1014 and 1046 (208); SEQ ID NOs: 1011, 1014 and 1047 (209); SEQ ID NOs: 1011, 1014 and 1048 (210); SEQ ID NOs: 1011, 1014 and 1049 (211); SEQ ID NOs: 1011, 1014 and 1050 (212); SEQ ID NOs: 1011, 1014 and 1051 (213); SEQ ID NOs: 1011, 1014 and 1052 (214); SEQ ID NOs: 1011, 1014 and 1053 (215); SEQ ID NOs: 1011, 1014 and 1054; (216); SEQ ID NOs: 1011, 1014 and 1055; (217) or SEQ ID NOs: 1011, 1014 and 1056; (218).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below: SEQ ID NOs: 1011, 1014 and 923 (172); SEQ ID NOs: 1011, 1014 and 971 (173); SEQ ID NOs: 1011, 1014 and 972; (174); SEQ ID NOs: 1011, 1014 and 1026 (189); SEQ ID NOs: 1011, 1014 and 1030 (192); SEQ ID NOs: 1011, 1014 and 1031 (193); SEQ ID NOs: 1011, 1014 and 1032 (194); SEQ ID NOs: 1011, 1014 and 1077 (220); SEQ ID NOs: 1011, 1014 and 1033 (195); SEQ ID NOs: 1011, 1014 and 1034 (196); SEQ ID NOs: 1011, 1014 and 1035 (197); SEQ ID NOs: 1011, 1014 and 1036 (198); SEQ ID NOs: 1011, 1014 and 1037 (199); SEQ ID NOs: 1011, 1014 and 1038 (200); SEQ ID NOs: 1011, 1014 and 1039 (201); SEQ ID NOs: 1011, 1014 and 1040 (202); SEQ ID NOs: 1011, 1014 and 1041 (203); SEQ ID NOs: 1011, 1014 and 1042 (204); SEQ ID NOs: 1011, 1014 and 1043 (205); SEQ ID NOs: 1011, 1014 and 1044 (206); SEQ ID NOs: 1011, 1014 and 1045 (207); SEQ ID NOs: 1011, 1014 and 1046 (208); SEQ ID NOs: 1011, 1014 and 1047 (209); SEQ ID NOs: 1011, 1014 and 1048 (210); SEQ ID NOs: 1011, 1014 and 1049 (211); SEQ ID NOs: 1011, 1014 and 1050 (212); SEQ ID NOs: 1011, 1014 and 1051 (213); SEQ ID NOs: 1011, 1014 and 1052 (214); SEQ ID NOs: 1011, 1014 and 1053 (215); SEQ ID NOs: 1011, 1014 and 1054 (216); SEQ ID NOs: 1011, 1014 and 1055 (217) or SEQ ID NOs: 1011, 1014 and 1056 (218) (CD4 D4-binding Fab SCT-1.22+IL-15v).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1089, 1090 and 1088 (246).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1009, 1010 and 923 (165).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1009, 1010 and 971 (166).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1009, 1010 and 972 (167).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1012 and 923 (168).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1012 and 971 (169).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1012 and 972 (170).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1012 and 974 (171).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 923 (172).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 971 (173).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 972 (174).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014 and 974 (175).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1015, 1016 and 923 (176).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1015, 1016 and 971 (177).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1015, 1017 and 971 (178).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1015, 1016 and 972 (179).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1015, 1017 and 972 (180).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1018, 1017 and 974 (181).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1018, 1017 and 985 (182).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1019, 1020 and 923 (183).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1019, 1020 and 971 (184).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1019, 1020 and 972 (185).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1021, 1020 and 974 (186).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1022, 1023 and 923 (187).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1024, 1025 and 923 (188).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1026 (189).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1027, 1014 and 1028 (190).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1027, 1014 and 1029 (191).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1030 (192).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1031 (193).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1032 (194).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1077 (220).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1033 (195).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1034 (196).

In some embodiments, the CD4-targeted or CD4-binding IL-15v molecule comprises a heavy chain (HC) and a light chain (LC) that bind to CD4 D3 and an IL15RA Sushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1035 (197).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1036 (198).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1037 (199).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1038 (200).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1039 (201).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1040 (202).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1041 (203).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1042 (204).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1043 (205).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1044 (206).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1045 (207).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1046 (208).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1047 (209).

In some embodiments, the CD4-targeted or CD4-binding IL-15v molecule comprises a heavy chain (HC) and a light chain (LC) that bind to CD4 D3 and an IL15RA Sushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1048 (210).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1049 (211).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1050 (212).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1051 (213).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1052 (214).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1053 (215).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1054 (216).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1055 (217).

In some embodiments, the CD4-targeted or CD4-binding IL-15v molecule comprises a heavy chain (HC) and a light chain (LC) that bind to CD4 D3 and an IL15RASushi-IL15v-Fc fusion protein comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1011, 1014 and 1056 (218).

### Four Polypeptide Format—Binds to CD4 D3

In some embodiments, the CD4-targeted or CD4-binding IL-15v molecule comprises four polypeptide chains: (i) a first immunoglobulin heavy chain (HC), (ii), a first immunoglobulin light chain (LC), wherein the first HC and first LC form a first CD4 binding domain, (iii) a second HC comprising a C-terminal IL15RASushi-IL15v, and (iv) a second LC, wherein the second HC and second LC form a second CD4 binding domain. In some embodiments, the first LC and the second LC have the same amino acid sequence.

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1061 and 1014 (222).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1062 and 1014 (223).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1063 and 1014 (224).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1064 and 1014 (225).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1065 and 1014 (226).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1066 and 1014 (227).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1067 and 1014 (228).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1068 and 1014 (229).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1069 and 1014 (230).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1013, 1014, 1070 and 1014 (231).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1071, 1017, 1072 and 1017 (232).

In some embodiments, the CD4-targeted or CD4-binding IL-15v molecule comprises (i) a first immunoglobulin heavy chain (HC), (ii), a first immunoglobulin light chain (LC), (iii) a second HC comprising a C-terminal IL15RASushi-IL15v, and (iv) a second LC, comprising, respectively, the amino acid sequences set forth in, or amino acid sequences at least 90%, 91%, 92%, 93%, 94%, 95%, 96%, 97%, 98%, 99% identical to, respectively, the full length of the amino acid sequences set forth in SEQ ID NOs: 1071, 1017, 1073 and 1017 (233).

TABLE G
amino acid sequences of illustrative CD4-targeted IL-15 molecules

| | | | |
|---|---|---|---|
| Molecule | | CD4-binding | IL-15Rα Sushi domain-IL-15 -Fc |
| Name | CD4-binding domain HC | domain LC | fusion protein |

Binds to CD4 D1

| | | | |
|---|---|---|---|
| 100 | SEQ ID NO: 921 | SEQ ID NO: 922 | SEQ ID NO: 923 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPSSKSTSGGTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 101 | SEQ ID NO: 924 | SEQ ID NO: 922 | SEQ ID NO: 925 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 102 | SEQ ID NO: 926 | SEQ ID NO: 922 | SEQ ID NO: 925 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 103 | SEQ ID NO: 926 | SEQ ID NO: 922 | SEQ ID NO: 1074 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHNTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 104 | SEQ ID NO: 926 | SEQ ID NO: 922 | SEQ ID NO: 1075 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVQDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQENST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 105 | SEQ ID NO: 926 | SEQ ID NO: 922 | SEQ ID NO: 1076 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESNVHPSCKVT |
| | PSVFPLAPCSRSTSESTAALGCLVKDYFPEP | SGSGTDYTLTISSLQP | AMKCFLLELQVISLESGDASIHDTVQDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQENST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 106 | SEQ ID NO: 926 | SEQ ID NO: 922 | SEQ ID NO: 974 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIALA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 107 | SEQ ID NO: 926 | SEQ ID NO: 929 | SEQ ID NO: 1076 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSILHPGVPSRFSG | SDLKKIEDLIQSMHIDATLYTESNVHPSCKVT |
| | PSVFPLAPCSRSTSESTAALGCLVKDYFPEP | SGSGTDYTLTISSLQP | AMKCFLLELQVISLESGDASIHDTVQDLIILA |
| | VTVSWNSGALTSGVHTFPAVLQSSGLYSLSS | EDFATYYCLQYDNPLQ | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 108 | SEQ ID NO: 927 | SEQ ID NO: 928 | SEQ ID NO: 925 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEYGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSILHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLF | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 109 | SEQ ID NO: 926 | SEQ ID NO: 929 | SEQ ID NO: 925 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSILHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLQ | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQENST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 110 | SEQ ID NO: 926 | SEQ ID NO: 922 | SEQ ID NO: 985 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGGGGGSGGGGSGGGGSGGGGSNWVNVI |
| | YCSRHYGGSYDPMDYWGQGTTVTVSSASTKG | HYTSTLHPGVPSRFSG | GDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 111 | SEQ ID NO: 930 | SEQ ID NO: 922 | SEQ ID NO: 923 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKGRFTISRDNSKNTLYLQMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPSSKSTSGGTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 112 | SEQ ID NO: 931 | SEQ ID NO: 922 | SEQ ID NO: 925 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKGRFTISRDNSKNTLYLQMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 113 | SEQ ID NO: 932 | SEQ ID NO: 922 | SEQ ID NO: 933 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKGRFTISRDNSKNTLYLQMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPSSKSTSGGTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLWCLVK | STLTLSKADYEKHKVY | SLSCAVKGFYPSDIAVEWESNGQPENNYKTTP |
| | GFYPSDIAVEWESNGQPENNYKTTPPVLDSD | ACEVTHQGLSSPVTKS | PVLDSDGSFFLVSKLTVDKSRWQQGNVFSCSV |
| | GSFFLYSKLTVDKSRWQQGNVFSCSVMHEAL | FNRGEC | MHEALHNRYTQKSLSLSPGK |
| | HNHYTQKSLSLSPGK | | |
| 114 | SEQ ID NO: 934 | SEQ ID NO: 922 | SEQ ID NO: 935 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEGGSTFYADS | DRVTITCRASQDINKY | FKRKAGTSSLTECVLNKATNVAHWTTPSLKCI |
| | VKGRFTISRDNSKNTLYLQMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLWCLVKGFY | STLTLSKADYEKHKVY | SLSCAVKGFYPSDIAVEWESNGQPENNYKTTP |
| | PSDIAVEWESNGQPENNYKTTPPVLDSDGSF | ACEVTHQGLSSPVTKS | PVLDSDGSFFLVSRLTVDKSRWQEGNVFSCSV |
| | FLYSRLTVDKSRWQEGNVFSCSVMHEALHNH | FNRGEC | MHEALHNRYTQKSLSLSLGK |
| | YTQKSLSLSLGK | | |
| 115 | SEQ ID NO: 936 | SEQ ID NO: 937 | SEQ ID NO: 923 |
| | EVQLVESGGGVVRPGGSLRLSCAPSGFTFDD | AIQMTQSPSSLSASVG | ITCPPPMSVEHADIWVKSYSLYSRERYICNSG |
| | YGMSWVRQAPGKGLEWVSGINWNGDSTGYAD | DRVTITCRASQDIRDD | FKRKAGTSSLTECVLNKATNVAHWTTPSLKCI |
| | SVRGRFTISRDNAKNSLYLQMNSLRAEDTAL | LGWYQQKPGKAPKLLI | RGGGGSGGGGSGGGGSGGGGSGGGGSNWVNVI |
| | YHCARDGAIGGMDVWGKGTTVTVSSASTKGP | YSASTLQSGVPSRFSG | SDLKKIEDLIQSMHIDATLYTESDVHPSCKVT |
| | SVFPLAPSSKSTSGGTAALGCLVKDYFPEPV | SESGTDFTLTISSLQP | AMKCFLLELQVISLESGDASIHDTVEDLIILA |
| | TVSWNSGALTSGVHTFPAVLQSSGLYSLSSV | EDFAAYYCLQDYNYPW | NNSLSSNGNVTESGCKECEELEEKNIKEFLQS |
| | VTVPSSSLGTQTYICNVNHKPSNTKVDKKVE | TFGQGTRVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 116 | SEQ ID NO: 938 | SEQ ID NO: 939 | SEQ ID NO: 923 |
| | QVQLVQSGAEVRKPGASVKVSCKVSGHTLTE | ETTLTQSPAFMSATPG | ITCPPPMSVEHADIWVKSYSLYSRERYICNSG |
| | LSMHWIRQAPGEGLEWMGYFDPRGGETIYAQ | DKVNISCKASQDIDDD | FKRKAGTSSLTECVLNKATNVAHWTTPSLKCI |
| | KFQGRVTLTEDTSTDTAYMELTSLTSEDTAV | MNWFQRKPGEAAIFII | RGGGGSGGGGSGGGGSGGGGSGGGGSNWVNVI |
| | YYCATGGDRDYYYYYMDVWGKGTTVTVSSAS | QEATTLVPGVPPRFSG | SDLKKIEDLIQSMHIDATLYTESDVHPSCKVT |
| | TKGPSVFPLAPSSKSTSGGTAALGCLVKDYF | SGYGTDFTLTISNIES | AMKCFLLELQVISLESGDASIHDTVEDLIILA |
| | PEPVTVSWNSGALTSGVHTFPAVLQSSGLYS | EDAAYYFCLQHDSFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 117 | SEQ ID NO: 940 | SEQ ID NO: 941 | SEQ ID NO: 923 |
| | QVQLLQSGAEVKRPGASVRVSCKVSGFTLTK | ETTLTQSPAFMSATPG | ITCPPPMSVEHADIWVKSYSLYSRERYICNSG |
| | LSMHWVRQAPGKGLEWMGFFDPRDGERIYAQ | DKVNISCKASQDIDDD | FKRKAGTSSLTECVLNKATNVAHWTTPSLKCI |
| | KFQGRVTMTEDTSTDTAYMELSSLRSEDTAV | LNWYQQKPGEAAIFIV | RGGGGSGGGGSGGGGSGGGGSGGGGSNWVNVI |
| | YYCATGGDYDYYYYYMGVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDETLTINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 118 | SEQ ID NO: 942 | SEQ ID NO: 943 | SEQ ID NO: 923 |
| | QVQLVQSGAEVKKPGASVKVSCKVSGHTVTE | ETTLTQSPAFMSATPG | ITCPPPMSVEHADIWVKSYSLYSRERYICNSG |
| | LSMHWVRQAPGKGLEWMGSFHPRDDEIIYAQ | DKVNISCKASQDIDDD | FKRKAGTSSLTECVLNKATNVAHWTTPSLKCI |
| | KFQGRVTMTEDSFTDTAYMELSSLTSEDTAV | MNWYQQKPGEAAIFII | RGGGGSGGGGSGGGGSGGGGSGGGGSNWVNVI |
| | YYCATGGNPDYYFYYMDVWGKGTPVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNFPL | NNSLSSNGNVTESGCKECEELEEKNIKEFLQS |
| | LSSVVTVPSSSLGTQTYICNVNHKPSNTKVD | TFGQGTKVEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 119 | SEQ ID NO: 944 | SEQ ID NO: 945 | SEQ ID NO: 923 |
| | QVQLLQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGSFHPRDGETIYAQ | DKVNISCKASQDIDDD | FKRKAGTSSLTECVLNKATNVAHWTTPSLKCI |
| | KFQGRVTLTEDTSKDTAFMELSRLRSEDTAV | MNWYQQKPGEAAIFII | RGGGGSGGGGSGGGGGGGGSGGGGSNWVNVI |
| | YYCATGGNKDYYFYYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNEPL | NNSLSSNGNVTESGCKECEELEEKNIKEFLQS |
| | LSSVVTVPSSSLGTQTYICNVNHKPSNTKVD | TFGGGTKVEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 120 | SEQ ID NO: 946 | SEQ ID NO: 947 | SEQ ID NO: 923 |
| | QVQLVQSGAEVKKPGASVKVSCKVSGYTLTA | ETTLTQSPAFMSATPG | ITCPPPMSVEHADIWVKSYSLYSRERYICNSG |
| | LSMHWVRQAPGKGLEWMGYENPRDGETIYAQ | DKVNISCKASQDIDDD | FKRKAGTSSLTECVLNKATNVAHWTTPSLKCI |
| | KFQGRVTMTEDTFTDTAYMELSSLRSEDTAI | MNWYQQKPGEAAIFII | RGGGGSGGGGSGGGGSGGGGSGGGGSNWVNVI |
| | YYCATGGDSDYYYYYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDETLIINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 121 | SEQ ID NO: 948 | SEQ ID NO: 949 | SEQ ID NO: 923 |
| | QVQLVQSGAEVKKPGASVKVSCKVSGHTLTE | ETTLTQSPAFMSATPG | ITCPPPMSVEHADIWVKSYSLYSRERYICNSG |
| | LSMHWVRQAPGTGLEWMGYENPRDDETIYTQ | DKVTISCKASQDIDDD | FKRKAGTSSLTECVLNKATNVAHWTTPSLKCI |
| | KFQGRVTMTEDTSTDTAYMELRSLRSEDTAV | MNWYQQKPGEAAIFII | RGGGGSGGGGSGGGGSGGGGSGGGGSNWVNVI |
| | YYCATGGNKDYYFYYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNFPL | NNSLSSNGNVTESGCKECEELEEKNIKEFLQS |
| | LSSVVTVPSSSLGTQTYICNVNHKPSNTKVD | TFGQGTKVEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 122 | SEQ ID NO: 950 | SEQ ID NO: 951 | SEQ ID NO: 923 |
| | QVQLVQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGVEDPKYGETTYAQ | DKVNISCKASQDIDDD | FKRKAGTSSLTECVLNKATNVAHWTTPSLKCI |
| | KFQDRVTMTEDTSTDTAYMELSSLRSEDTAV | MNWYQQKPGEAAIFII | RGGGGSGGGGSGGGGSGGGGSGGGGSNWVNVI |
| | YYCATGGNYEYFYYYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 123 | SEQ ID NO: 952 | SEQ ID NO: 953 | SEQ ID NO: 923 |
| | QVQLIQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGIFDPKYGETMYAQ | DKVNISCKASQDIDDD | FKRKAGTSSLTECVLNKATNVAHWTTPSLKCI |
| | KFQGRVTLTEDTSTDTAYMELSSLRSEDTAV | MNWYQQKPGEAAIFVI | RGGGGSGGGGSGGGGSGGGGSGGGGSNWVNVI |
| | YYCATGGSPDYFYYYMDVWGKGTTVTVSSAS | QEATTLVPGIPRRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 124 | SEQ ID NO: 954 | SEQ ID NO: 955 | SEQ ID NO: 923 |
| | QVQLLQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGIFDPRNGETIYAQ | DKVNISCKASQDIDDD | FKRKAGTSSLTECVLNKATNVAHWTTPSLKCI |
| | KFQGRVTMTEDTSTDTAYMELSSLRSEDTAV | MNWYQQKPGEPAIFII | RGGGGSGGGGGGGGSGGGGSGGGGSNWVNVI |
| | YYCATGGSPDYYYWYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDETLTINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 125 | SEQ ID NO: 956 | SEQ ID NO: 955 | SEQ ID NO: 923 |
| | QVQLLQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGIFDPRNGETIYAQ | DKVNISCKASQDIDDD | FKRKAGTSSLTECVLNKATNVAHWTTPSLKCI |
| | KFQGRVTMTEDTSTDTAYMELSSLSSEDTAV | MNWYQQKPGEPAIFII | RGGGGSGGGGSGGGGSGGGGSGGGGSNWVNVI |
| | YYCATGGSPDYYFWYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDFTLTINNIES | AMKCFLLELQVISLESGDASIHDTVEDLIILA |
| | PEPVTVSWNSGALTSGVHTFPAVLQSSGLYS | EDAAYYFCLQHDNFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 126 | SEQ ID NO: 957 | SEQ ID NO: 958 | SEQ ID NO: 923 |
| | QVQLVQSGAEVKKPGASVKVSCKASGYTFTS | EIVMTQSPATLSLSPG | ITCPPPMSVEHADIWVKSYSLYSRERYICNSG |
| | YDINWVRQATGQGLEWMGWMNPNSGNTGSTQ | ERATLSCRASQSISSS | FKRKAGTSSLTECVLNKATNVAHWTTPSLKCI |
| | KFQGRVTMTRNTSITTAYMELSSLRSEDTAV | YLSWYQQKPGQAPRLL | RGGGGSGGGGSGGGGSGGGGSGGGGSNWVNVI |
| | YYCARGVLKGDYYYMDVWGKGTTVTVSSAST | IYGSSTRATGIPARFS | SDLKKIEDLIQSMHIDATLYTESDVHPSCKVT |
| | KGPSVFPLAPSSKSTSGGTAALGCLVKDYFP | GSGSGTDFTLTISSLQ | AMKCFLLELQVISLESGDASIHDTVEDLIILA |
| | EPVTVSWNSGALTSGVHTFPAVLQSSGLYSL | PEDFAVYYCQQDDHLP | NNSLSSNGNVTESGCKECEELEEKNIKEFLQS |
| | SSVVTVPSSSLGTQTYICNVNHKPSNTKVDK | LTFGGGTKVEIKRTVA | FVHIVQMFINTSTSGGSDKTHTCPPCPAPEAA |
| | KVEPKSCDKTHTCPPCPAPEAAGGPSVFLFP | APSVFIFPPSDEQLKS | GGPSVFLFPPKPKDTLMISRTPEVTCVVVDVS |
| | PKPKDTLMISRTPEVTCVVVDVSHEDPEVKF | GTASVVCLLNNFYPRE | HEDPEVKFNWYVDGVEVHNAKTKPREEQYNST |
| | NWYVDGVEVHNAKTKPREEQYNSTYRVVSVL | AKVQWKVDNALQSGNS | YRVVSVLTVLHQDWLNGKEYKCKVSNKALPAS |
| | TVLHQDWLNGKEYKCKVSNKALPASIEKTIS | QESVTEQDSKDSTYSL | IEKTISKAKGQPREPQVYTLPPSREEMTKNQV |
| | KAKGQPREPQVYTLPPSREEMTKNQVSLSCA | SSTLTLSKADYEKHKV | SLWCLVKGFYPSDIAVEWESNGQPENNYKTTP |
| | VKGFYPSDIAVEWESNGQPENNYKTTPPVLD | YACEVTHQGLSSPVTK | PVLDSDGSFFLYSKLTVDKSRWQQGNVFSCSV |
| | SDGSFFLVSKLTVDKSRWQQGNVFSCSVMHE | SFNRGEC | MHEALHNHYTQKSLSLSPGK |
| | ALHNRYTQKSLSLSPGK | | |
| 127 | SEQ ID NO: 959 | SEQ ID NO: 960 | SEQ ID NO: 923 |
| | QVQLVHSGAEVKKPGASVKVSCKASGYTFTS | EIVMTQSPATLSLSPG | ITCPPPMSVEHADIWVKSYSLYSRERYICNSG |
| | YDINWVRQATGQGLEWMGWVNPNSGNTGSTQ | ERATLSCRASQSISST | FKRKAGTSSLTECVLNKATNVAHWTTPSLKCI |
| | KFQGRVTMTRNTSISTAYMELSSLRSEDTAV | YLSWYQQKPGQAPRLL | RGGGGSGGGGSGGGGSGGGGSGGGGSNWVNVI |
| | YYCARGVLKGDYYYMDVWGKGTTVTVSSAST | IYGSSTRATGIPARFS | SDLKKIEDLIQSMHIDATLYTESDVHPSCKVT |
| | KGPSVFPLAPSSKSTSGGTAALGCLVKDYFP | GSGSGTDFTLTISSLQ | AMKCFLLELQVISLESGDASIHDTVEDLIILA |
| | EPVTVSWNSGALTSGVHTFPAVLQSSGLYSL | PEDFAVYYCQQDDHLP | NNSLSSNGNVTESGCKECEELEEKNIKEFLQS |
| | SSVVTVPSSSLGTQTYICNVNHKPSNTKVDK | LTFGGGTKVEIKRTVA | FVHIVQMFINTSTSGGSDKTHTCPPCPAPEAA |
| | KVEPKSCDKTHTCPPCPAPEAAGGPSVFLFP | APSVFIFPPSDEQLKS | GGPSVFLFPPKPKDTLMISRTPEVTCVVVDVS |
| | PKPKDTLMISRTPEVTCVVVDVSHEDPEVKF | GTASVVCLLNNFYPRE | HEDPEVKFNWYVDGVEVHNAKTKPREEQYNST |
| | NWYVDGVEVHNAKTKPREEQYNSTYRVVSVL | AKVQWKVDNALQSGNS | YRVVSVLTVLHQDWLNGKEYKCKVSNKALPAS |
| | TVLHQDWLNGKEYKCKVSNKALPASIEKTIS | QESVTEQDSKDSTYSL | IEKTISKAKGQPREPQVYTLPPSREEMTKNQV |
| | KAKGQPREPQVYTLPPSREEMTKNQVSLSCA | SSTLTLSKADYEKHKV | SLWCLVKGFYPSDIAVEWESNGQPENNYKTTP |
| | VKGFYPSDIAVEWESNGQPENNYKTTPPVLD | YACEVTHQGLSSPVTK | PVLDSDGSFFLYSKLTVDKSRWQQGNVFSCSV |
| | SDGSFFLVSKLTVDKSRWQQGNVFSCSVMHE | SFNRGEC | MHEALHNHYTQKSLSLSPGK |
| | ALHNRYTQKSLSLSPGK | | |
| 128 | SEQ ID NO: 961 | SEQ ID NO: 962 | SEQ ID NO: 923 |
| | QVQLVQSGAEVKKPGASVKVSCKASGYTFTS | EIVMTQSPATLSLSPG | ITCPPPMSVEHADIWVKSYSLYSRERYICNSG |
| | YDINWVRQATGQGLEWMGWVNPNSGNTGSTQ | ERATLSCRASQSISST | FKRKAGTSSLTECVLNKATNVAHWTTPSLKCI |
| | KFQGRVTMTRNTSISTAYMELSSLRSEDTAV | YLSWYQQKPGQAPRLL | RGGGGSGGGGSGGGGSGGGGSGGGGSNWVNVI |
| | YYCARGVLKGDYYYMDVWGKGTTVTVSSAST | IYGSSTRATGIPARFR | SDLKKIEDLIQSMHIDATLYTESDVHPSCKVT |
| | KGPSVFPLAPSSKSTSGGTAALGCLVKDYFP | GSGSGTDFTLTISSLQ | AMKCFLLELQVISLESGDASIHDTVEDLIILA |
| | EPVTVSWNSGALTSGVHTFPAVLQSSGLYSL | PEDFAVYYCQQDDHLP | NNSLSSNGNVTESGCKECEELEEKNIKEFLQS |
| | SSVVTVPSSSLGTQTYICNVNHKPSNTKVDK | LTFGGGTKVEIKRTVA | FVHIVQMFINTSTSGGSDKTHTCPPCPAPEAA |
| | KVEPKSCDKTHTCPPCPAPEAAGGPSVFLFP | APSVFIFPPSDEQLKS | GGPSVFLFPPKPKDTLMISRTPEVTCVVVDVS |
| | PKPKDTLMISRTPEVTCVVVDVSHEDPEVKF | GTASVVCLLNNFYPRE | HEDPEVKFNWYVDGVEVHNAKTKPREEQYNST |
| | NWYVDGVEVHNAKTKPREEQYNSTYRVVSVL | AKVQWKVDNALQSGNS | YRVVSVLTVLHQDWLNGKEYKCKVSNKALPAS |
| | TVLHQDWLNGKEYKCKVSNKALPASIEKTIS | QESVTEQDSKDSTYSL | IEKTISKAKGQPREPQVYTLPPSREEMTKNQV |
| | KAKGQPREPQVYTLPPSREEMTKNQVSLSCA | SSTLTLSKADYEKHKV | SLWCLVKGFYPSDIAVEWESNGQPENNYKTTP |
| | VKGFYPSDIAVEWESNGQPENNYKTTPPVLD | YACEVTHQGLSSPVTK | PVLDSDGSFFLYSKLTVDKSRWQQGNVFSCSV |
| | SDGSFFLVSKLTVDKSRWQQGNVFSCSVMHE | SFNRGEC | MHEALHNHYTQKSLSLSPGK |
| | ALHNRYTQKSLSLSPGK | | |
| 129 | SEQ ID NO: 961 | SEQ ID NO: 960 | SEQ ID NO: 923 |
| | QVQLVQSGAEVKKPGASVKVSCKASGYTFTS | EIVMTQSPATLSLSPG | ITCPPPMSVEHADIWVKSYSLYSRERYICNSG |
| | YDINWVRQATGQGLEWMGWVNPNSGNTGSTQ | ERATLSCRASQSISST | FKRKAGTSSLTECVLNKATNVAHWTTPSLKCI |
| | KFQGRVTMTRNTSISTAYMELSSLRSEDTAV | YLSWYQQKPGQAPRLL | RGGGGSGGGGSGGGGSGGGGSGGGGSNWVNVI |
| | YYCARGVLKGDYYYMDVWGKGTTVTVSSAST | IYGSSTRATGIPARFS | SDLKKIEDLIQSMHIDATLYTESDVHPSCKVT |
| | KGPSVFPLAPSSKSTSGGTAALGCLVKDYFP | GSGSGTDFTLTISSLQ | AMKCFLLELQVISLESGDASIHDTVEDLIILA |
| | EPVTVSWNSGALTSGVHTFPAVLQSSGLYSL | PEDFAVYYCQQDDHLP | NNSLSSNGNVTESGCKECEELEEKNIKEFLQS |
| | SSVVTVPSSSLGTQTYICNVNHKPSNTKVDK | LTFGGGTKVEIKRTVA | FVHIVQMFINTSTSGGSDKTHTCPPCPAPEAA |
| | KVEPKSCDKTHTCPPCPAPEAAGGPSVFLFP | APSVFIFPPSDEQLKS | GGPSVFLFPPKPKDTLMISRTPEVTCVVVDVS |
| | PKPKDTLMISRTPEVTCVVVDVSHEDPEVKF | GTASVVCLLNNFYPRE | HEDPEVKFNWYVDGVEVHNAKTKPREEQYNST |
| | NWYVDGVEVHNAKTKPREEQYNSTYRVVSVL | AKVQWKVDNALQSGNS | YRVVSVLTVLHQDWLNGKEYKCKVSNKALPAS |
| | TVLHQDWLNGKEYKCKVSNKALPASIEKTIS | QESVTEQDSKDSTYSL | IEKTISKAKGQPREPQVYTLPPSREEMTKNQV |
| | KAKGQPREPQVYTLPPSREEMTKNQVSLSCA | SSTLTLSKADYEKHKV | SLWCLVKGFYPSDIAVEWESNGQPENNYKTTP |
| | VKGFYPSDIAVEWESNGQPENNYKTTPPVLD | YACEVTHQGLSSPVTK | PVLDSDGSFFLYSKLTVDKSRWQQGNVFSCSV |
| | SDGSFFLVSKLTVDKSRWQQGNVFSCSVMHE | SFNRGEC | MHEALHNHYTQKSLSLSPGK |
| | ALHNRYTQKSLSLSPGK | | |
| 249 | SEQ ID NO: 926 | SEQ ID NO: 922 | SEQ ID NO: 1091 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEALIALA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 251 | SEQ ID NO: 926 | SEQ ID NO: 922 | SEQ ID NO: 1092 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHNTVEDLIALA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 252 | SEQ ID NO: 926 | SEQ ID NO: 922 | SEQ ID NO: 1093 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVGVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 253 | SEQ ID NO: 926 | SEQ ID NO: 922 | SEQ ID NO: 1094 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVDVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 254 | SEQ ID NO: 926 | SEQ ID NO: 922 | SEQ ID NO: 1095 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVENLIALA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 255 | SEQ ID NO: 926 | SEQ ID NO: 922 | SEQ ID NO: 1096 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESNVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 259 | SEQ ID NO: 926 | SEQ ID NO: 922 | SEQ ID NO: 1097 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVE | IAWYQQKPGKGPKLLI | RGGGGSGGGGSGGGGSGGGGSGGGGSNWVGVI |
| | YCSRHYGGSYDPMDYWGQGTTVTVSSASTKG | HYTSTLHPGVPSRFSG | GDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 260 | SEQ ID NO: 926 | SEQ ID NO: 922 | SEQ ID NO: 1098 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVADLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 261 | SEQ ID NO: 926 | SEQ ID NO: 922 | SEQ ID NO: 1099 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHATVEDLIILA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 262 | SEQ ID NO: 926 | SEQ ID NO: 922 | SEQ ID NO: 1100 |
| | EVQLLESGGGLVQPGGSLRLSCAASGFSFSN | DIQMTQSPSSLSASVG | ITCPPPMSVEHADIWVKSYSLYSRERYICNSG |
| | YAMSWVRQAPGKGLEWVASINEWGSTFYADS | DRVTITCRASQDINKY | FKRKAGTSSLTECVLNKATNVAHWTTPSLKCI |
| | VKSRFTISRDNSKNTLYLEMNSLRAEDTAVF | IAWYQQKPGKGPKLLI | RGGGGSGGGGSGGGGSGGGGSGGGGSNWVNVI |
| | YCSRHYGGSYDPMDYWGQGTTVTVSSASTKG | HYTSTLHPGVPSRFSG | SDLKKIEDLIQSMHIDATLYTESDVHPSCKVT |
| | PSVFPLAPCSRSTSESTAALGCLVKDYFPEP | SGSGTDYTLTISSLQP | AMKCFLLELQVISLESGDASIHDTVQDLIALA |
| | VTVSWNSGALTSGVHTFPAVLQSSGLYSLSS | EDFATYYCLQYDNPLY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |

Binds to CD4 D2

| | | | |
|---|---|---|---|
| 245 | SEQ ID NO: 1086 | SEQ ID NO: 1087 | SEQ ID NO: 1088 |
| | QVQLQQSGPEVVKPGASVKMSCKASGYTFTS | DIVMTQSPDSLAVSLG | ITCPPPMSVEHADIWVKSYSLYSRERYICNSG |
| | YVIHWVRQKPGQGLDWIGYINPYNDGTDYDE | ERVTMNCKSSQSLLYS | FKRKAGTSSLTECVLNKATNVAHWTTPSLKCI |
| | KFKGKATLTSDTSTSTAYMELSSLRSEDTAV | TNQKNYLAWYQQKPGQ | RGGGGSGGGGSGGGGSGGGGSGGGGSNWVDVI |
| | YYCAREKDNYATGAWFAYWGQGTLVTVSSAS | SPKLLIYWASTRFSGV | SDLKKIEDLIQSMHIDATLYTESDVHPSCKVT |
| | TKGPSVFPLAPSSKSTSGGTAALGCLVKDYF | PDRFSGSGSGTDFTLT | AMKCFLLELQVISLESGDASIHDTVEDLIILA |
| | PEPVTVSWNSGALTSGVHTFPAVLQSSGLYS | ISSVQAEDVAVYYCQQ | NNSLSSNGNVTESGCKECEELEEKNIKEFLQS |
| | LSSVVTVPSSSLGTQTYICNVNHKPSNTKVD | YYSYRTFGGGTKLEIK | FVHIVQMFINTSTSGGSDKTHTCPPCPAPEAA |
| | KKVEPKSCDKTHTCPPCPAPEAAGGPSVFLF | RTVAAPSVFIFPPSDE | GGPSVFLFPPKPKDTLMISRTPEVTCVVVDVS |
| | PPKPKDTLMISRTPEVTCVVVDVSHEDPEVK | QLKSGTASVVCLLNNF | HEDPEVKFNWYVDGVEVHNAKTKPREEQYNST |
| | FNWYVDGVEVHNAKTKPREEQYNSTYRVVSV | YPREAKVQWKVDNALQ | YRVVSVLTVLHQDWLNGKEYKCKVSNKALPAS |
| | LTVLHQDWLNGKEYKCKVSNKALPASIEKTI | SGNSQESVTEQDSKDS | IEKTISKAKGQPREPQVYTLPPSREEMTKNQV |
| | SKAKGQPREPQVYTLPPSREEMTKNQVSLSC | TYSLSSTLTLSKADYE | SLWCLVKGFYPSDIAVEWESNGQPENNYKTTP |
| | AVKGFYPSDIAVEWESNGQPENNYKTTPPVL | KHKVYACEVTHQGLSS | PVLDSDGSFFLYSKLTVDKSRWQQGNVFSCSV |
| | DSDGSFFLVSKLTVDKSRWQQGNVFSCSVMH | PVTKSFNRGEC | MHEALHNHYTQKSLSLSPGK |
| | EALHNRYTQKSLSLSPGK | | |
| 130 | SEQ ID NO: 963 | SEQ ID NO: 964 | SEQ ID NO: 923 |
| | QVQLVQSGAEVKKPGASVKVSCKVSGYTLTE | DIVMTQTPLSLSVTPG | ITCPPPMSVEHADIWVKSYSLYSRERYICNSG |
| | LSMHWVRQAPEKGLEWMGGFDPEDGKTIYAP | QPASISCKSSQSLLHT | FKRKAGTSSLTECVLNKATNVAHWTTPSLKCI |
| | KFQGRVTMTEDTSTDTAYMDLSSLRSEDTAV | DGKTYLYWYLQKPGQP | RGGGGSGGGGSGGGGSGGGGSGGGGSNWVNVI |
| | YYCATGHNWNDGYYFYYYMDLWGKGTTVTVS | PHLLIYEVSNRFSGVP | SDLKKIEDLIQSMHIDATLYTESDVHPSCKVT |
| | SASTKGPSVFPLAPSSKSTSGGTAALGCLVK | DRLSGSGSGTDFTLKI | AMKCFLLELQVISLESGDASIHDTVEDLIILA |
| | DYFPEPVTVSWNSGALTSGVHTFPAVLQSSG | SRVEAEDVGVYYCMQS | NNSLSSNGNVTESGCKECEELEEKNIKEFLQS |
| | LYSLSSVVTVPSSSLGTQTYICNVNHKPSNT | LQLPLTFGGGTMVEIK | FVHIVQMFINTSTSGGSDKTHTCPPCPAPEAA |
| | KVDKKVEPKSCDKTHTCPPCPAPEAAGGPSV | RTVAAPSVFIFPPSDE | GGPSVFLFPPKPKDTLMISRTPEVTCVVVDVS |
| | FLFPPKPKDTLMISRTPEVTCVVVDVSHEDP | QLKSGTASVVCLLNNE | HEDPEVKFNWYVDGVEVHNAKTKPREEQYNST |
| | EVKFNWYVDGVEVHNAKTKPREEQYNSTYRV | YPREAKVQWKVDNALQ | YRVVSVLTVLHQDWLNGKEYKCKVSNKALPAS |
| | VSVLTVLHQDWLNGKEYKCKVSNKALPASIE | SGNSQESVTEQDSKDS | IEKTISKAKGQPREPQVYTLPPSREEMTKNQV |
| | KTISKAKGQPREPQVYTLPPSREEMTKNQVS | TYSLSSTLTLSKADYE | SLWCLVKGFYPSDIAVEWESNGQPENNYKTTP |
| | LSCAVKGFYPSDIAVEWESNGQPENNYKTTP | KHKVYACEVTHQGLSS | PVLDSDGSFFLYSKLTVDKSRWQQGNVFSCSV |
| | PVLDSDGSFFLVSKLTVDKSRWQQGNVFSCS | PVTKSFNRGEC | MHEALHNHYTQKSLSLSPGK |
| | VMHEALHNRYTQKSLSLSPGK | | |
| 131 | SEQ ID NO: 965 | SEQ ID NO: 966 | SEQ ID NO: 923 |
| | QVQLVESGGDSVKPGGSLRLSCAASGFTFSD | EIVMTQSPATLSVSPG | ITCPPPMSVEHADIWVKSYSLYSRERYICNSG |
| | YYMNWIRQAPGKGLEWISYISSSGNTIFYVD | ERATLSCRASQSVSRK | FKRKAGTSSLTECVLNKATNVAHWTTPSLKCI |
| | SVEGRFTVSRDNAKNSLYLQMNSLRAEDTAV | LAWYQQKPGQAPRLLI | RGGGGSGGGGSGGGGSGGGGSGGGGSNWVNVI |
| | YYCAREGYNNYNYSYYYFMDVWGKGTTVTVS | YAASTRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYFCQQYNNWPY | NNSLSSNGNVTESGCKECEELEEKNIKEFLQS |
| | LYSLSSVVTVPSSSLGTQTYICNVNHKPSNT | TFGRGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 132 | SEQ ID NO: 967 | SEQ ID NO: 968 | SEQ ID NO: 923 |
| | QVQLVESGGGVVQPGRSLRLSCAASGFTVSS | AIQMTQSPSSLSASVG | ITCPPPMSVEHADIWVKSYSLYSRERYICNSG |
| | FGMHWVRQAPGKGLEWVAIIWYDGTNKYYAD | DRVTFTCRASQGIRSG | FKRKAGTSSLTECVLNKATNVAHWTTPSLKCI |
| | SVKGRFTISRDNSKNTLYLQLNSLRGEDTAV | LGWYQQKPGKAPNLLI | RGGGGSGGGGSGGGGSGGGGSGGGGSNWVNVI |
| | YYCAREIAVDGTDYYMDVWGRGTTVTVSSAS | YAASTLQSGVPSRFSG | SDLKKIEDLIQSMHIDATLYTESDVHPSCKVT |
| | TKGPSVFPLAPSSKSTSGGTAALGCLVKDYF | SGSGTDFTLTITSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 133 | SEQ ID NO: 969 | SEQ ID NO: 970 | SEQ ID NO: 923 |
| | QVQLVESGGGLVTPGGSLRLSCAASGFTFSD | EIVMTQSPATLSVSPG | ITCPPPMSVEHADIWVKSYSLYSRERYICNSG |
| | YYMNWIRQAPGKGLEWLSYISSSGLTIFYVD | ERATLSCRASQSVSSN | FKRKAGTSSLTECVLNKATNVAHWTTPSLKCI |
| | SVKGRFTVSRDNAKNSLYLQMNSLRAEDTAV | LAWYQQKPGQAPRLLI | RGGGGSGGGGSGGGGSGGGGSGGGGSNWVNVI |
| | YYCAREGYSGFDDHYYYYTDVWGKGTTVTVS | YGASIRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCQHYKNWPY | NNSLSSNGNVTESGCKECEELEEKNIKEFLQS |
| | LYSLSSVVTVPSSSLGTQTYICNVNHKPSNT | TFGQGTRLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 134 | SEQ ID NO: 969 | SEQ ID NO: 970 | SEQ ID NO: 971 |
| | QVQLVESGGGLVTPGGSLRLSCAASGFTFSD | EIVMTQSPATLSVSPG | ITCPPPMSVEHADIWVKSYSLYSRERYICNSG |
| | YYMNWIRQAPGKGLEWLSYISSSGLTIFYVD | ERATLSCRASQSVSSN | FKRKAGTSSLTECVLNKATNVAHWTTPSLKCI |
| | SVKGRFTVSRDNAKNSLYLQMNSLRAEDTAV | LAWYQQKPGQAPRLLI | RGGGGSGGGGSGGGGSGGGGSGGGGSNWVNVI |
| | YYCAREGYSGFDDHYYYYTDVWGKGTTVTVS | YGASIRATGIPARFSG | GDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCQHYKNWPY | NNSLSSNGNVTESGCKECEELEEKNIKEFLQS |
| | LYSLSSVVTVPSSSLGTQTYICNVNHKPSNT | TFGQGTRLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 135 | SEQ ID NO: 969 | SEQ ID NO: 970 | SEQ ID NO: 972 |
| | QVQLVESGGGLVTPGGSLRLSCAASGFTFSD | EIVMTQSPATLSVSPG | ITCPPPMSVEHADIWVKSYSLYSRERYICNSG |
| | YYMNWIRQAPGKGLEWLSYISSSGLTIFYVD | ERATLSCRASQSVSSN | FKRKAGTSSLTECVLNKATNVAHWTTPSLKCI |
| | SVKGRFTVSRDNAKNSLYLQMNSLRAEDTAV | LAWYQQKPGQAPRLLI | RGGGGSGGGGSGGGGSGGGGSGGGGSNWVNVI |
| | YYCAREGYSGFDDHYYYYTDVWGKGTTVTVS | YGASIRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCQHYKNWPY | NNSLSSNGNVTESGCKECEELEEKNIKEFLQS |
| | LYSLSSVVTVPSSSLGTQTYICNVNHKPSNT | TFGQGTRLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 136 | SEQ ID NO: 973 | SEQ ID NO: 970 | SEQ ID NO: 974 |
| | QVQLVESGGGLVTPGGSLRLSCAASGFTFSD | EIVMTQSPATLSVSPG | ITCPPPMSVEHADIWVKSYSLYSRERYICNSG |
| | YYMNWIRQAPGKGLEWLSYISSSGLTIFYVD | ERATLSCRASQSVSSN | FKRKAGTSSLTECVLNKATNVAHWTTPSLKCI |
| | SVKGRFTVSRDNAKNSLYLQMNSLRAEDTAV | LAWYQQKPGQAPRLLI | RGGGGSGGGGSGGGGSGGGGSGGGGSNWVNVI |
| | YYCAREGYSGFDDHYYYYTDVWGKGTTVTVS | YGASIRATGIPARFSG | SDLKKIEDLIQSMHIDATLYTESDVHPSCKVT |
| | SASTKGPSVFPLAPCSRSTSESTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCQHYKNWPY | NNSLSSNGNVTESGCKECEELEEKNIKEFLQS |
| | LYSLSSVVTVPSSSLGTKTYTCNVDHKPSNT | TFGQGTRLEIKRTVAA | FVHIVQMFINTSGGGGGKYGPPCPPCPAPEAA |
| | KVDKRVESKYGPPCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSQEDPEVQ | TASVVCLLNNFYPREA | QEDPEVQFNWYVDGVEVHNAKTKPREEQENST |
| | FNWYVDGVEVHNAKTKPREEQFNSTYRVVSV | KVQWKVDNALQSGNSQ | YRVVSVLTVLHQDWLNGKEYKCKVSNKGLPSS |
| | LTVLHQDWLNGKEYKCKVSNKGLPSSIEKTI | ESVTEQDSKDSTYSLS | IEKTISKAKGQPREPQVYTLPPSQEEMTKNQV |
| | SKAKGQPREPQVYTLPPSQEEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSRLTVDKSRWQEGNVFSCSV |
| | DSDGSFFLVSRLTVDKSRWQEGNVFSCSVMH | FNRGEC | MHEALHNHYTQKSLSLSLGK |
| | EALHNRYTQKSLSLSLGK | | |
| 137 | SEQ ID NO: 975 | SEQ ID NO: 976 | SEQ ID NO: 923 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGSTIFYTD | ERATLSCRASQSVSSK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YNASTRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCQQYNNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 138 | SEQ ID NO: 977 | SEQ ID NO: 978 | SEQ ID NO: 923 |
| | QVQLVESGGGLVKPGGSLRLSCSASGFTFSD | EIVMTQSPAALSVSPG | ITCPPPMSVEHADIWVKSYSLYSRERYICNSG |
| | YYMNWIRQAPGKGLEWVSYISSSGITIFYTD | ERATLSCRASQSVSNK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDHYYYYTGVWGKGTTVTVS | YNASTRATSIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCQHYNNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 139 | SEQ ID NO: 977 | SEQ ID NO: 978 | SEQ ID NO: 971 |
| | QVQLVESGGGLVKPGGSLRLSCSASGFTFSD | EIVMTQSPAALSVSPG | ITCPPPMSVEHADIWVKSYSLYSRERYICNSG |
| | YYMNWIRQAPGKGLEWVSYISSSGITIFYTD | ERATLSCRASQSVSNK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDHYYYYTGVWGKGTTVTVS | YNASTRATSIPARFSG | GDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCQHYNNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 140 | SEQ ID NO: 977 | SEQ ID NO: 978 | SEQ ID NO: 972 |
| | QVQLVESGGGLVKPGGSLRLSCSASGFTFSD | EIVMTQSPAALSVSPG | ITCPPPMSVEHADIWVKSYSLYSRERYICNSG |
| | YYMNWIRQAPGKGLEWVSYISSSGITIFYTD | ERATLSCRASQSVSNK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDHYYYYTGVWGKGTTVTVS | YNASTRATSIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCQHYNNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 141 | SEQ ID NO: 977 | SEQ ID NO: 979 | SEQ ID NO: 972 |
| | QVQLVESGGGLVKPGGSLRLSCSASGFTFSD | EIVMTQSPAALSVSPG | ITCPPPMSVEHADIWVKSYSLYSRERYICNSG |
| | YYMNWIRQAPGKGLEWVSYISSSGITIFYTD | ERATLSCRASQSVSNK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDHYYYYTGVWGKGTTVTVS | YGASTRATSIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCQHYNNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 142 | SEQ ID NO: 980 | SEQ ID NO: 979 | SEQ ID NO: 974 |
| | QVQLVESGGGLVKPGGSLRLSCSASGFTFSD | EIVMTQSPAALSVSPG | ITCPPPMSVEHADIWVKSYSLYSRERYICNSG |
| | YYMNWIRQAPGKGLEWVSYISSSGITIFYTD | ERATLSCRASQSVSNK | FKRKAGTSSLTECVLNKATNVAHWTTPSLKCI |
| | SVKGRFTISRDNAKNSLYLQMNSLRAEDTAV | FAWYQQKPGQAPRLLI | RGGGGSGGGGSGGGGSGGGGSGGGGSNWVNVI |
| | YYCAREDYSNYEDHYYYYTGVWGKGTTVTVS | YGASTRATSIPARFSG | SDLKKIEDLIQSMHIDATLYTESDVHPSCKVT |
| | SASTKGPSVFPLAPCSRSTSESTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCQHYNNWPY | NNSLSSNGNVTESGCKECEELEEKNIKEFLQS |
| | LYSLSSVVTVPSSSLGTKTYTCNVDHKPSNT | TFGQGTKLEIKRTVAA | FVHIVQMFINTSGGGGGKYGPPCPPCPAPEAA |
| | KVDKRVESKYGPPCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSQEDPEVQ | TASVVCLLNNFYPREA | QEDPEVQFNWYVDGVEVHNAKTKPREEQFNST |
| | FNWYVDGVEVHNAKTKPREEQFNSTYRVVSV | KVQWKVDNALQSGNSQ | YRVVSVLTVLHQDWLNGKEYKCKVSNKGLPSS |
| | LTVLHQDWLNGKEYKCKVSNKGLPSSIEKTI | ESVTEQDSKDSTYSLS | IEKTISKAKGQPREPQVYTLPPSQEEMTKNQV |
| | SKAKGQPREPQVYTLPPSQEEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSRLTVDKSRWQEGNVFSCSV |
| | DSDGSFFLVSRLTVDKSRWQEGNVFSCSVMH | FNRGEC | MHEALHNHYTQKSLSLSLGK |
| | EALHNRYTQKSLSLSLGK | | |
| 143 | SEQ ID NO: 981 | SEQ ID NO: 982 | SEQ ID NO: 923 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YNASTRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 144 | SEQ ID NO: 981 | SEQ ID NO: 982 | SEQ ID NO: 971 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YNASTRATGIPARFSG | GDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 145 | SEQ ID NO: 981 | SEQ ID NO: 982 | SEQ ID NO: 972 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YNASTRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 146 | SEQ ID NO: 981 | SEQ ID NO: 983 | SEQ ID NO: 972 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGGGGGSGGGGSGGGGSNWVNVI |
| | YYCAREDYSNYEDYYYYYTGVWGKGTTVTVS | YGASTRATGIPARFSG | SDLKKIEDLIQSMHIDATLYTESDVHPSCKVT |
| | SASTKGPSVFPLAPSSKSTSGGTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 147 | SEQ ID NO: 984 | SEQ ID NO: 983 | SEQ ID NO: 974 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YGASTRATGIPARFSG | SDLKKIEDLIQSMHIDATLYTESDVHPSCKVT |
| | SASTKGPSVFPLAPCSRSTSESTAALGCLVK | SGSGTEFTLTISSLQS | AMKCFLLELQVISLESGDASIHDTVEDLIALA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTKTYTCNVDHKPSNT | TFGQGTKLEIKRTVAA | FVHIVQMFINTSGGGGGKYGPPCPPCPAPEAA |
| | KVDKRVESKYGPPCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSQEDPEVQ | TASVVCLLNNFYPREA | QEDPEVQFNWYVDGVEVHNAKTKPREEQFNST |
| | FNWYVDGVEVHNAKTKPREEQFNSTYRVVSV | KVQWKVDNALQSGNSQ | YRVVSVLTVLHQDWLNGKEYKCKVSNKGLPSS |
| | LTVLHQDWLNGKEYKCKVSNKGLPSSIEKTI | ESVTEQDSKDSTYSLS | IEKTISKAKGQPREPQVYTLPPSQEEMTKNQV |
| | SKAKGQPREPQVYTLPPSQEEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSRLTVDKSRWQEGNVFSCSV |
| | DSDGSFFLVSRLTVDKSRWQEGNVFSCSVMH | FNRGEC | MHEALHNHYTQKSLSLSLGK |
| | EALHNRYTQKSLSLSLGK | | |
| 148 | SEQ ID NO: 984 | SEQ ID NO: 983 | SEQ ID NO: 985 |
| | QVQLVESGGGLVKPGGSLRLSCAASGFTFSD | EIVMTQSPAALSVSPG | ITCPPPMSVEHADIWVKSYSLYSRERYICNSG |
| | YYMNWIRQAPGKGLEWVSYISSSGKTIFYTD | ERATLSCRASQSVSSK | FKRKAGTSSLTECVLNKATNVAHWTTPSLKCI |
| | SVKGRFTISRDNAKNSLFLQMNSLRAEDTAV | FAWYQQKPGQAPRLLI | RGGGGSGGGGSGGGGSGGGGSGGGGSNWVNVI |
| | YYCAREDYSNYEDYYYYYTGVWGKGTTVTVS | YGASTRATGIPARFSG | GDLKKIEDLIQSMHIDATLYTESDVHPSCKVT |
| | SASTKGPSVFPLAPCSRSTSESTAALGCLVK | SGSGTEFTLTISSLQS | AMKCFLLELQVISLESGDASIHDTVEDLIILA |
| | DYFPEPVTVSWNSGALTSGVHTFPAVLQSSG | EDFAVYYCHQYKNWPY | NNSLSSNGNVTESGCKECEELEEKNIKEFLQS |
| | LYSLSSVVTVPSSSLGTKTYTCNVDHKPSNT | TFGQGTKLEIKRTVAA | FVHIVQMFINTSGGGGGKYGPPCPPCPAPEAA |
| | KVDKRVESKYGPPCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSQEDPEVQ | TASVVCLLNNFYPREA | QEDPEVQFNWYVDGVEVHNAKTKPREEQENST |
| | FNWYVDGVEVHNAKTKPREEQFNSTYRVVSV | KVQWKVDNALQSGNSQ | YRVVSVLTVLHQDWLNGKEYKCKVSNKGLPSS |
| | LTVLHQDWLNGKEYKCKVSNKGLPSSIEKTI | ESVTEQDSKDSTYSLS | IEKTISKAKGQPREPQVYTLPPSQEEMTKNQV |
| | SKAKGQPREPQVYTLPPSQEEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSRLTVDKSRWQEGNVFSCSV |
| | DSDGSFFLVSRLTVDKSRWQEGNVFSCSVMH | FNRGEC | MHEALHNHYTQKSLSLSLGK |
| | EALHNRYTQKSLSLSLGK | | |
| 149 | SEQ ID NO: 986 | SEQ ID NO: 987 | SEQ ID NO: 923 |
| | QVQLVESGGGLVKPGGSLRLSCAASGFTFSD | EILMTQSPATLSVSPG | ITCPPPMSVEHADIWVKSYSLYSRERYICNSG |
| | YYMNWIRQAPGKGLEWISYISISGQTIYYGD | ERTTLSCRASQSISSN | FKRKAGTSSLTECVLNKATNVAHWTTPSLKCI |
| | SVKGRFTVSRDNAKNSLFLEMNSLRAEDSAV | LAWYQQNPGQAPRLLI | RGGGGSGGGGSGGGGSGGGGSGGGGSNWVNVI |
| | YFCAREGYSNYGVKYYYYMDVWGKGTTVTVS | YNASTRATDIPARFSG | SDLKKIEDLIQSMHIDATLYTESDVHPSCKVT |
| | SASTKGPSVFPLAPSSKSTSGGTAALGCLVK | GGSGTEFTLTISSLQS | AMKCFLLELQVISLESGDASIHDTVEDLIILA |
| | DYFPEPVTVSWNSGALTSGVHTFPAVLQSSG | EDFAVYYCQQYNAWTY | NNSLSSNGNVTESGCKECEELEEKNIKEFLQS |
| | LYSLSSVVTVPSSSLGTQTYICNVNHKPSNT | TFGQGTKLEIKRTVAA | FVHIVQMFINTSTSGGSDKTHTCPPCPAPEAA |
| | KVDKKVEPKSCDKTHTCPPCPAPEAAGGPSV | PSVFIFPPSDEQLKSG | GGPSVFLFPPKPKDTLMISRTPEVTCVVVDVS |
| | FLFPPKPKDTLMISRTPEVTCVVVDVSHEDP | TASVVCLLNNFYPREA | HEDPEVKFNWYVDGVEVHNAKTKPREEQYNST |
| | EVKFNWYVDGVEVHNAKTKPREEQYNSTYRV | KVQWKVDNALQSGNSQ | YRVVSVLTVLHQDWLNGKEYKCKVSNKALPAS |
| | VSVLTVLHQDWLNGKEYKCKVSNKALPASIE | ESVTEQDSKDSTYSLS | IEKTISKAKGQPREPQVYTLPPSREEMTKNQV |
| | KTISKAKGQPREPQVYTLPPSREEMTKNQVS | STLTLSKADYEKHKVY | SLWCLVKGFYPSDIAVEWESNGQPENNYKTTP |
| | LSCAVKGFYPSDIAVEWESNGQPENNYKTTP | ACEVTHQGLSSPVTKS | PVLDSDGSFFLYSKLTVDKSRWQQGNVFSCSV |
| | PVLDSDGSFFLVSKLTVDKSRWQQGNVFSCS | FNRGEC | MHEALHNHYTQKSLSLSPGK |
| | VMHEALHNRYTQKSLSLSPGK | | |
| 150 | SEQ ID NO: 988 | SEQ ID NO: 989 | SEQ ID NO: 923 |
| | QVQLVESGGGLVKPGGSLRLSCAASGFTFSD | EIMMTQSPATLSVSPG | ITCPPPMSVEHADIWVKSYSLYSRERYICNSG |
| | HYMNWVRQVPGKGLEWISYISISGNTIYYTD | ERATLSCRASQSVSSN | FKRKAGTSSLTECVLNKATNVAHWTTPSLKCI |
| | SVKGRFTVSRDNAKNSLYLQMTSLRAEDTAV | LAWYQQKPGQAPRLLI | RGGGGSGGGGSGGGGSGGGGSGGGGSNWVNVI |
| | YYCVREGYSSSSRGDYSYYTDVWGKGTTVTV | YGASTRATGIPARFSG | SDLKKIEDLIQSMHIDATLYTESDVHPSCKVT |
| | SSASTKGPSVFPLAPSSKSTSGGTAALGCLV | SGSGTEFTLTISSLQS | AMKCFLLELQVISLESGDASIHDTVEDLIILA |
| | KDYFPEPVTVSWNSGALTSGVHTFPAVLQSS | EDFAVYYCQQYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 151 | SEQ ID NO: 988 | SEQ ID NO: 989 | SEQ ID NO: 971 |
| | QVQLVESGGGLVKPGGSLRLSCAASGFTFSD | EIMMTQSPATLSVSPG | ITCPPPMSVEHADIWVKSYSLYSRERYICNSG |
| | HYMNWVRQVPGKGLEWISYISISGNTIYYTD | ERATLSCRASQSVSSN | FKRKAGTSSLTECVLNKATNVAHWTTPSLKCI |
| | SVKGRFTVSRDNAKNSLYLQMTSLRAEDTAV | LAWYQQKPGQAPRLLI | RGGGGSGGGGSGGGGSGGGGSGGGGSNWVNVI |
| | YYCVREGYSSSSRGDYSYYTDVWGKGTTVTV | YGASTRATGIPARFSG | GDLKKIEDLIQSMHIDATLYTESDVHPSCKVT |
| | SSASTKGPSVFPLAPSSKSTSGGTAALGCLV | SGSGTEFTLTISSLQS | AMKCFLLELQVISLESGDASIHDTVEDLIILA |
| | KDYFPEPVTVSWNSGALTSGVHTFPAVLQSS | EDFAVYYCQQYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 152 | SEQ ID NO: 988 | SEQ ID NO: 989 | SEQ ID NO: 972 |
| | QVQLVESGGGLVKPGGSLRLSCAASGFTFSD | EIMMTQSPATLSVSPG | ITCPPPMSVEHADIWVKSYSLYSRERYICNSG |
| | HYMNWVRQVPGKGLEWISYISISGNTIYYTD | ERATLSCRASQSVSSN | FKRKAGTSSLTECVLNKATNVAHWTTPSLKCI |
| | SVKGRFTVSRDNAKNSLYLQMTSLRAEDTAV | LAWYQQKPGQAPRLLI | RGGGGSGGGGSGGGGSGGGGSGGGGSNWVNVI |
| | YYCVREGYSSSSRGDYSYYTDVWGKGTTVTV | YGASTRATGIPARFSG | SDLKKIEDLIQSMHIDATLYTESDVHPSCKVT |
| | SSASTKGPSVFPLAPSSKSTSGGTAALGCLV | SGSGTEFTLTISSLQS | AMKCFLLELQVISLESGDASIHDTVEDLIALA |
| | KDYFPEPVTVSWNSGALTSGVHTFPAVLQSS | EDFAVYYCQQYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 153 | SEQ ID NO: 990 | SEQ ID NO: 991 | SEQ ID NO: 923 |
| | QVQLVESGGGLVKPGGSLRLSCAASGFTFSD | EIVMTQSPATLSASPG | ITCPPPMSVEHADIWVKSYSLYSRERYICNSG |
| | YYMNWVRQVPGKGLEWISYISISGRTIYYID | ERVNLSCRASQGVSSN | FKRKAGTSSLTECVLNKATNVAHWTTPSLKCI |
| | SVKGRFTVSRDNAKNSLYLQMTNLRAEDTAV | LAWYQQKVGQAPRLLI | RGGGGSGGGGSGGGGSGGGGSGGGGSNWVNVI |
| | YYCVREGYSSSSRGDYSYYTDVWGKGTTVTV | YGASTRATDIPARFSG | SDLKKIEDLIQSMHIDATLYTESDVHPSCKVT |
| | SSASTKGPSVFPLAPSSKSTSGGTAALGCLV | SGSGSEFTLTISSLQS | AMKCFLLELQVISLESGDASIHDTVEDLIILA |
| | KDYFPEPVTVSWNSGALTSGVHTFPAVLQSS | EDFAVYYCQHYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 154 | SEQ ID NO: 990 | SEQ ID NO: 991 | SEQ ID NO: 971 |
| | QVQLVESGGGLVKPGGSLRLSCAASGFTFSD | EIVMTQSPATLSASPG | ITCPPPMSVEHADIWVKSYSLYSRERYICNSG |
| | YYMNWVRQVPGKGLEWISYISISGRTIYYID | ERVNLSCRASQGVSSN | FKRKAGTSSLTECVLNKATNVAHWTTPSLKCI |
| | SVKGRFTVSRDNAKNSLYLQMTNLRAEDTAV | LAWYQQKVGQAPRLLI | RGGGGSGGGGSGGGGSGGGGSGGGGSNWVNVI |
| | YYCVREGYSSSSRGDYSYYTDVWGKGTTVTV | YGASTRATDIPARFSG | GDLKKIEDLIQSMHIDATLYTESDVHPSCKVT |
| | SSASTKGPSVFPLAPSSKSTSGGTAALGCLV | SGSGSEFTLTISSLQS | AMKCFLLELQVISLESGDASIHDTVEDLIILA |
| | KDYFPEPVTVSWNSGALTSGVHTFPAVLQSS | EDFAVYYCQHYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 155 | SEQ ID NO: 990 | SEQ ID NO: 991 | SEQ ID NO: 972 |
| | QVQLVESGGGLVKPGGSLRLSCAASGFTFSD | EIVMTQSPATLSASPG | ITCPPPMSVEHADIWVKSYSLYSRERYICNSG |
| | YYMNWVRQVPGKGLEWISYISISGRTIYYID | ERVNLSCRASQGVSSN | FKRKAGTSSLTECVLNKATNVAHWTTPSLKCI |
| | SVKGRFTVSRDNAKNSLYLQMTNLRAEDTAV | LAWYQQKVGQAPRLLI | RGGGGSGGGGSGGGGSGGGGSGGGGSNWVNVI |
| | YYCVREGYSSSSRGDYSYYTDVWGKGTTVTV | YGASTRATDIPARFSG | SDLKKIEDLIQSMHIDATLYTESDVHPSCKVT |
| | SSASTKGPSVFPLAPSSKSTSGGTAALGCLV | SGSGSEFTLTISSLQS | AMKCFLLELQVISLESGDASIHDTVEDLIALA |
| | KDYFPEPVTVSWNSGALTSGVHTFPAVLQSS | EDFAVYYCQHYKNWPY | NNSLSSNGNVTESGCKECEELEEKNIKEFLQS |
| | GLYSLSSVVTVPSSSLGTQTYICNVNHKPSN | TFGQGTKLEIKRTVAA | FVHIVQMFINTSTSGGSDKTHTCPPCPAPEAA |
| | TKVDKKVEPKSCDKTHTCPPCPAPEAAGGPS | PSVFIFPPSDEQLKSG | GGPSVFLFPPKPKDTLMISRTPEVTCVVVDVS |
| | VFLFPPKPKDTLMISRTPEVTCVVVDVSHED | TASVVCLLNNFYPREA | HEDPEVKFNWYVDGVEVHNAKTKPREEQYNST |
| | PEVKFNWYVDGVEVHNAKTKPREEQYNSTYR | KVQWKVDNALQSGNSQ | YRVVSVLTVLHQDWLNGKEYKCKVSNKALPAS |
| | VVSVLTVLHQDWLNGKEYKCKVSNKALPASI | ESVTEQDSKDSTYSLS | IEKTISKAKGQPREPQVYTLPPSREEMTKNQV |
| | EKTISKAKGQPREPQVYTLPPSREEMTKNQV | STLTLSKADYEKHKVY | SLWCLVKGFYPSDIAVEWESNGQPENNYKTTP |
| | SLSCAVKGFYPSDIAVEWESNGQPENNYKTT | ACEVTHQGLSSPVTKS | PVLDSDGSFFLYSKLTVDKSRWQQGNVFSCSV |
| | PPVLDSDGSFFLVSKLTVDKSRWQQGNVFSC | FNRGEC | MHEALHNHYTQKSLSLSPGK |
| | SVMHEALHNRYTQKSLSLSPGK | | |
| 156 | SEQ ID NO: 992 | SEQ ID NO: 991 | SEQ ID NO: 974 |
| | QVQLVESGGGLVKPGGSLRLSCAASGFTFSD | EIVMTQSPATLSASPG | ITCPPPMSVEHADIWVKSYSLYSRERYICNSG |
| | YYMNWVRQVPGKGLEWISYISISGRTIYYID | ERVNLSCRASQGVSSN | FKRKAGTSSLTECVLNKATNVAHWTTPSLKCI |
| | SVKGRFTVSRDNAKNSLYLQMTNLRAEDTAV | LAWYQQKVGQAPRLLI | RGGGGSGGGGSGGGGSGGGGSGGGGSNWVNVI |
| | YYCVREGYSSSSRGDYSYYTDVWGKGTTVTV | YGASTRATDIPARFSG | SDLKKIEDLIQSMHIDATLYTESDVHPSCKVT |
| | SSASTKGPSVFPLAPCSRSTSESTAALGCLV | SGSGSEFTLTISSLQS | AMKCFLLELQVISLESGDASIHDTVEDLIALA |
| | KDYFPEPVTVSWNSGALTSGVHTFPAVLQSS | EDFAVYYCQHYKNWPY | NNSLSSNGNVTESGCKECEELEEKNIKEFLQS |
| | GLYSLSSVVTVPSSSLGTKTYTCNVDHKPSN | TFGQGTKLEIKRTVAA | FVHIVQMFINTSGGGGGKYGPPCPPCPAPEAA |
| | TKVDKRVESKYGPPCPPCPAPEAAGGPSVFL | PSVFIFPPSDEQLKSG | GGPSVFLFPPKPKDTLMISRTPEVTCVVVDVS |
| | FPPKPKDTLMISRTPEVTCVVVDVSQEDPEV | TASVVCLLNNFYPREA | QEDPEVQFNWYVDGVEVHNAKTKPREEQENST |
| | QFNWYVDGVEVHNAKTKPREEQFNSTYRVVS | KVQWKVDNALQSGNSQ | YRVVSVLTVLHQDWLNGKEYKCKVSNKGLPSS |
| | VLTVLHQDWLNGKEYKCKVSNKGLPSSIEKT | ESVTEQDSKDSTYSLS | IEKTISKAKGQPREPQVYTLPPSQEEMTKNQV |
| | ISKAKGQPREPQVYTLPPSQEEMTKNQVSLS | STLTLSKADYEKHKVY | SLWCLVKGFYPSDIAVEWESNGQPENNYKTTP |
| | CAVKGFYPSDIAVEWESNGQPENNYKTTPPV | ACEVTHQGLSSPVTKS | PVLDSDGSFFLYSRLTVDKSRWQEGNVFSCSV |
| | LDSDGSFFLVSRLTVDKSRWQEGNVFSCSVM | FNRGEC | MHEALHNHYTQKSLSLSLGK |
| | HEALHNRYTQKSLSLSLGK | | |

Binds to CD4 D2 and/or D3

| | | | |
|---|---|---|---|
| 157 | SEQ ID NO: 993 | SEQ ID NO: 994 | SEQ ID NO: 923 |
| | QVQLVESGGGVVQPGRSLRLSCAASGFTVSS | AIQMTQSPSSLSASVG | ITCPPPMSVEHADIWVKSYSLYSRERYICNSG |
| | YGMHWVRQAPGKGLEWVAIIWFDGSNKYYAD | DRVTITCRASQDIRNG | FKRKAGTSSLTECVLNKATNVAHWTTPSLKCI |
| | SLTGLFTISRDSSKNTLFLQMNSLSLEDTAV | LGWYQQKPGKAPKLLI | RGGGGSGGGGGGGGSGGGGSGGGGSNWVNVI |
| | YYCAREVALEGYYYYMDVWGKGTTVTVSSAS | YTTSSLQTGVPSRFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 158 | SEQ ID NO: 995 | SEQ ID NO: 996 | SEQ ID NO: 923 |
| | QVQLVESGGGVVQPGRSLSLSCAASGFTVSS | AIQMTQSPSSLSASVG | ITCPPPMSVEHADIWVKSYSLYSRERYICNSG |
| | YGMHWVRQAPGKGLEWVAILWFDGSNKFYAD | DRVTITCRASQDIRNG | FKRKAGTSSLTECVLNKATNVAHWTTPSLKCI |
| | SVKGRFTISRDNSKNTLYLQMNSLRVEDTAV | LGWYQQNPGKAPKLLI | RGGGGGGGGSGGGGSGGGGSGGGGSNWVNVI |
| | YYCARELALEGYYYYMDVWGKGTTVTVSSAS | YTTSSLQSGVPSRFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 159 | SEQ ID NO: 997 | SEQ ID NO: 998 | SEQ ID NO: 923 |
| | QVQLVESGGGVVQPGRSLRLSCAASGFTVSS | AIQMTQSPSSLSASVG | ITCPPPMSVEHADIWVKSYSLYSRERYICNSG |
| | YGMHWVRQAPGKGLEWVAILWEDGSNQFYAD | DRVTITCRSSQDIRNG | FKRKAGTSSLTECVLNKATNVAHWTTPSLKCI |
| | SVKGRFTISRDNSKNTLYLQMNSLRVEDTAV | LGWYQQKPGKAPKLLI | RGGGGSGGGGSGGGGSGGGGSGGGGSNWVNVI |
| | YYCAREIALEGYYYYMDVWGKGTTVTVSSAS | YTTSSLQSGVPSRFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 160 | SEQ ID NO: 999 | SEQ ID NO: 1000 | SEQ ID NO: 923 |
| | QVQLVESGGGVVQPGRALRLSCAASGFTFSS | DIQMTQSPSSLSASVG | ITCPPPMSVEHADIWVKSYSLYSRERYICNSG |
| | YGMHWVRQAPGKGLEWVAIIWYDGSNKYYAD | DRVTITCRASQDIRNG | FKRKAGTSSLTECVLNKATNVAHWTTPSLKCI |
| | SVKGRFTISRDNSKNTLYLQMNSLRAEDTAV | LGWYQQKPGKAPKLLI | RGGGGSGGGGSGGGGSGGGGSGGGGSNWVNVI |
| | YYCAREVALEGYYYYMDVWGKGTTVTVSSAS | YTTSNLQSGVPSRFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 161 | SEQ ID NO: 1001 | SEQ ID NO: 1002 | SEQ ID NO: 923 |
| | QVQLVESGGGVVQPGRSLRLSCAASGFTFSS | AIQMTQSPSSLSASVG | ITCPPPMSVEHADIWVKSYSLYSRERYICNSG |
| | YGMHWVRQAPGKGLEWVAIIWYDGNNKYYAD | DRVTITCRASQGIRNG | FKRKAGTSSLTECVLNKATNVAHWTTPSLKCI |
| | SVKGRFTISRDNSKNTLYLQMNSLRAEDTAV | LGWYQLKPGKAPKLLI | RGGGGSGGGGSGGGGSGGGGSGGGGSNWVNVI |
| | YYCTREVALEGYYYYMDVWGKGTTVTVSSAS | YAASSLQSGVPSSFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | EDFATYYCLQDYNYPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 162 | SEQ ID NO: 1003 | SEQ ID NO: 1004 | SEQ ID NO: 923 |
| | EVQLLESGGGLVQPGGSLRLSCAASGFTFSS | AIQMTQSPSSLSAFVG | ITCPPPMSVEHADIWVKSYSLYSRERYICNSG |
| | YAMSWVRQAPGKGLEWVSGISGSVGSTYYAD | DRVTITCRASQGIRND | FKRKAGTSSLTECVLNKATNVAHWTTPSLKCI |
| | SVKGRFTISRDKSKNTLYLQMNSLRAEDTAV | LGWYQQKPGKAPKLLI | RGGGGSGGGGSGGGGSGGGGSGGGGSNWVNVI |
| | YYCAKEGYDWSGMDVWGKGTTVTVSSASTKG | YAASSLQSGVPSRFSG | SDLKKIEDLIQSMHIDATLYTESDVHPSCKVT |
| | PSVFPLAPSSKSTSGGTAALGCLVKDYFPEP | SGSGTDFTLTISSLQP | AMKCFLLELQVISLESGDASIHDTVEDLIILA |
| | VTVSWNSGALTSGVHTFPAVLQSSGLYSLSS | EDFATYYCLQDYNYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 163 | SEQ ID NO: 1005 | SEQ ID NO: 1006 | SEQ ID NO: 923 |
| | QVLLVESGGGVVQPGRSLRLSCAASGFTFSS | AIQMTQSPSSLSASVG | ITCPPPMSVEHADIWVKSYSLYSRERYICNSG |
| | YGMHWVRQAPGKGLEWVTLIWFDGTTRFYAD | DRVTITCRASQGIRNG | FKRKAGTSSLTECVLNKATNVAHWTTPSLKCI |
| | SVKGRFTVSRDNSKKTLYLQMNSLRAEDTAV | LGWYQQKPGKAPKLLI | RGGGGSGGGGSGGGGSGGGGSGGGGSNWVNVI |
| | YYCAREVAIQGRDYYIDVWGKGTTVTVSSAS | YAASSLQSGVPSRFSG | SDLKKIEDLIQSMHIDATLYTESDVHPSCKVT |
| | TKGPSVFPLAPSSKSTSGGTAALGCLVKDYF | SGSGTDFTLTISSLQP | AMKCFLLELQVISLESGDASIHDTVEDLIILA |
| | PEPVTVSWNSGALTSGVHTFPAVLQSSGLYS | ADFATYYCLQDYNYPY | NNSLSSNGNVTESGCKECEELEEKNIKEFLQS |
| | LSSVVTVPSSSLGTQTYICNVNHKPSNTKVD | TFGQGTKLEIRRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 164 | SEQ ID NO: 1007 | SEQ ID NO: 1008 | SEQ ID NO: 923 |
| | QVQLVESGGGVVQPGRSLRLSCAASGFTFSS | AIQMTQSPSSLSASVG | ITCPPPMSVEHADIWVKSYSLYSRERYICNSG |
| | YGIHWFRQAPGKGLEWVAIIWEDGINKFYAD | DRVTITCRASQDIRNG | FKRKAGTSSLTECVLNKATNVAHWTTPSLKCI |
| | SVKGRFTISRDNSKNTVNLQMNSLRVEDTAI | LGWYQQKPGKAPMLLI | RGGGGSGGGGSGGGGSGGGGSGGGGSNWVNVI |
| | YYCAREVAIQGYYYYMDVWGNGTTVTVSSAS | YTTSNLQNGVPSRFSG | SDLKKIEDLIQSMHIDATLYTESDVHPSCKVT |
| | TKGPSVFPLAPSSKSTSGGTAALGCLVKDYF | SRSGTDFTLTISGLQP | AMKCFLLELQVISLESGDASIHDTVEDLIILA |
| | PEPVTVSWNSGALTSGVHTFPAVLQSSGLYS | EDFAAYFCLQDYNYPY | NNSLSSNGNVTESGCKECEELEEKNIKEFLQS |
| | LSSVVTVPSSSLGTQTYICNVNHKPSNTKVD | TFGQGTKLDVKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |

Binds to CD4 D3

| | | | |
|---|---|---|---|
| 246 | SEQ ID NO: 1089 | SEQ ID NO: 1090 | SEQ ID NO: 1088 |
| | QIQLVQSGPELKKPGETVKISCKASGYTFTN | NIVLTQSPASLAVSLG | ITCPPPMSVEHADIWVKSYSLYSRERYICNSG |
| | YGMNWVKQAPGKGLKCMGWINTNTGEPTYAE | QRATISCRASESVDSY | FKRKAGTSSLTECVLNKATNVAHWTTPSLKCI |
| | EFKGRFAFSLETSATTAFLQINNLKDEDTAT | GNSFMHWYQQKPGQPP | RGGGGSGGGGSGGGGSGGGGSGGGGSNWVDVI |
| | YFCARLGIYYDYGYYAMDYWGQGASVTVSSA | KLFIYLASNLESGVPA | SDLKKIEDLIQSMHIDATLYTESDVHPSCKVT |
| | STKGPSVFPLAPSSKSTSGGTAALGCLVKDY | RFSGSGSRTDFTLTID | AMKCFLLELQVISLESGDASIHDTVEDLIILA |
| | FPEPVTVSWNSGALTSGVHTFPAVLQSSGLY | PVEADDAATYYCQQNN | NNSLSSNGNVTESGCKECEELEEKNIKEFLQS |
| | SLSSVVTVPSSSLGTQTYICNVNHKPSNTKV | EDPYTFGGGTKLEIKR | FVHIVQMFINTSTSGGSDKTHTCPPCPAPEAA |
| | DKKVEPKSCDKTHTCPPCPAPEAAGGPSVFL | TVAAPSVFIFPPSDEQ | GGPSVFLFPPKPKDTLMISRTPEVTCVVVDVS |
| | FPPKPKDTLMISRTPEVTCVVVDVSHEDPEV | LKSGTASVVCLLNNFY | HEDPEVKFNWYVDGVEVHNAKTKPREEQYNST |
| | KFNWYVDGVEVHNAKTKPREEQYNSTYRVVS | PREAKVQWKVDNALQS | YRVVSVLTVLHQDWLNGKEYKCKVSNKALPAS |
| | VLTVLHQDWLNGKEYKCKVSNKALPASIEKT | GNSQESVTEQDSKDST | IEKTISKAKGQPREPQVYTLPPSREEMTKNQV |
| | ISKAKGQPREPQVYTLPPSREEMTKNQVSLS | YSLSSTLTLSKADYEK | SLWCLVKGFYPSDIAVEWESNGQPENNYKTTP |
| | CAVKGFYPSDIAVEWESNGQPENNYKTTPPV | HKVYACEVTHQGLSSP | PVLDSDGSFFLYSKLTVDKSRWQQGNVFSCSV |
| | LDSDGSFFLVSKLTVDKSRWQQGNVFSCSVM | VTKSFNRGEC | MHEALHNHYTQKSLSLSPGK |
| | HEALHNRYTQKSLSLSPGK | | |
| 165 | SEQ ID NO: 1009 | SEQ ID NO: 1010 | SEQ ID NO: 923 |
| | EVQLLESGGGLVQPGGSLRLSCAASEIIFST | AIQMTQSPYSLSASVG | ITCPPPMSVEHADIWVKSYSLYSRERYICNSG |
| | YAMSWVRQAPGKGLEWVSGISGSGDNTYYAD | DRVTITCRASQGIRND | FKRKAGTSSLTECVLNKATNVAHWTTPSLKCI |
| | SVKGRFTISSDNSKNTLYLQMNSLRAEDTAV | LGWYQQKPGKAPKVLI | RGGGGSGGGGSGGGGSGGGGSGGGGSNWVNVI |
| | YYCVKEGYNWNYMDVWGKGTTVTVSSASTKG | YEASSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 166 | SEQ ID NO: 1009 | SEQ ID NO: 1010 | SEQ ID NO: 971 |
| | EVQLLESGGGLVQPGGSLRLSCAASEIIFST | AIQMTQSPYSLSASVG | ITCPPPMSVEHADIWVKSYSLYSRERYICNSG |
| | YAMSWVRQAPGKGLEWVSGISGSGDNTYYAD | DRVTITCRASQGIRND | FKRKAGTSSLTECVLNKATNVAHWTTPSLKCI |
| | SVKGRFTISSDNSKNTLYLQMNSLRAEDTAV | LGWYQQKPGKAPKVLI | RGGGGSGGGGSGGGGSGGGGSGGGGSNWVNVI |
| | YYCVKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 167 | SEQ ID NO: 1009 | SEQ ID NO: 1010 | SEQ ID NO: 972 |
| | EVQLLESGGGLVQPGGSLRLSCAASEIIFST | AIQMTQSPYSLSASVG | ITCPPPMSVEHADIWVKSYSLYSRERYICNSG |
| | YAMSWVRQAPGKGLEWVSGISGSGDNTYYAD | DRVTITCRASQGIRND | FKRKAGTSSLTECVLNKATNVAHWTTPSLKCI |
| | SVKGRFTISSDNSKNTLYLQMNSLRAEDTAV | LGWYQQKPGKAPKVLI | RGGGGSGGGGSGGGGSGGGGSGGGGSNWVNVI |
| | YYCVKEGYNWNYMDVWGKGTTVTVSSASTKG | YEASSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIALA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 168 | SEQ ID NO: 1011 | SEQ ID NO: 1012 | SEQ ID NO: 923 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | SETSSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 169 | SEQ ID NO: 1011 | SEQ ID NO: 1012 | SEQ ID NO: 971 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | SETSSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 170 | SEQ ID NO: 1011 | SEQ ID NO: 1012 | SEQ ID NO: 972 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | SETSSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIALA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 171 | SEQ ID NO: 1013 | SEQ ID NO: 1012 | SEQ ID NO: 974 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | SETSSLQSGVPSRFSG | SDLKKIEDLIQSMHIDATLYTESDVHPSCKVT |
| | PSVFPLAPCSRSTSESTAALGCLVKDYFPEP | SRSGTDFTLTISSLQP | AMKCFLLELQVISLESGDASIHDTVEDLIALA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 172 | SEQ ID NO: 1011 | SEQ ID NO: 1014 | SEQ ID NO: 923 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 173 | SEQ ID NO: 1011 | SEQ ID NO: 1014 | SEQ ID NO: 971 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 174 | SEQ ID NO: 1011 | SEQ ID NO: 1014 | SEQ ID NO: 972 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | SDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIALA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 175 | SEQ ID NO: 1013 | SEQ ID NO: 1014 | SEQ ID NO: 974 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | SDLKKIEDLIQSMHIDATLYTESDVHPSCKVT |
| | PSVFPLAPCSRSTSESTAALGCLVKDYFPEP | SRSGTDFTLTISSLQP | AMKCFLLELQVISLESGDASIHDTVEDLIALA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTKTYTCNVDHKPSNTKVDKRV | TFGQGTKLEIKRTVAA | FVHIVQMFINTSGGGGGKYGPPCPPCPAPEAA |
| | ESKYGPPCPPCPAPEAAGGPSVFLFPPKPKD | PSVFIFPPSDEQLKSG | GGPSVFLFPPKPKDTLMISRTPEVTCVVVDVS |
| | TLMISRTPEVTCVVVDVSQEDPEVQFNWYVD | TASVVCLLNNFYPREA | QEDPEVQFNWYVDGVEVHNAKTKPREEQFNST |
| | GVEVHNAKTKPREEQFNSTYRVVSVLTVLHQ | KVQWKVDNALQSGNSQ | YRVVSVLTVLHQDWLNGKEYKCKVSNKGLPSS |
| | DWLNGKEYKCKVSNKGLPSSIEKTISKAKGQ | ESVTEQDSKDSTYSLS | IEKTISKAKGQPREPQVYTLPPSQEEMTKNQV |
| | PREPQVYTLPPSQEEMTKNQVSLSCAVKGFY | STLTLSKADYEKHKVY | SLWCLVKGFYPSDIAVEWESNGQPENNYKTTP |
| | PSDIAVEWESNGQPENNYKTTPPVLDSDGSF | ACEVTHQGLSSPVTKS | PVLDSDGSFFLYSRLTVDKSRWQEGNVFSCSV |
| | FLVSRLTVDKSRWQEGNVFSCSVMHEALHNR | FNRGEC | MHEALHNHYTQKSLSLSLGK |
| | YTQKSLSLSLGK | | |
| 176 | SEQ ID NO: 1015 | SEQ ID NO: 1016 | SEQ ID NO: 923 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWCQQKPGKAPKFLI | RGGGGSGGGGSGGGGSGGGGSGGGGSNWVNVI |
| | YYCTKDNEDYYMDVWGKGTTVTVSSASTKGP | YAASTLQSGVPSRFSG | SDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 177 | SEQ ID NO: 1015 | SEQ ID NO: 1016 | SEQ ID NO: 971 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWCQQKPGKAPKELI | RGGGGSGGGGSGGGGSGGGGSGGGGSNWVNVI |
| | YYCTKDNEDYYMDVWGKGTTVTVSSASTKGP | YAASTLQSGVPSRFSG | GDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 178 | SEQ ID NO: 1015 | SEQ ID NO: 1017 | SEQ ID NO: 971 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWYQQKPGKAPKFLI | RGGGGSGGGGSGGGGSGGGGSGGGGSNWVNVI |
| | YYCTKDNEDYYMDVWGKGTTVTVSSASTKGP | YAASTLQSGVPSRFSG | GDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 179 | SEQ ID NO: 1015 | SEQ ID NO: 1016 | SEQ ID NO: 972 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWCQQKPGKAPKFLI | RGGGGSGGGGSGGGGSGGGGSGGGGSNWVNVI |
| | YYCTKDNEDYYMDVWGKGTTVTVSSASTKGP | YAASTLQSGVPSRFSG | SDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIALA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 180 | SEQ ID NO: 1015 | SEQ ID NO: 1017 | SEQ ID NO: 972 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWYQQKPGKAPKFLI | RGGGGSGGGGSGGGGSGGGGSGGGGSNWVNVI |
| | YYCTKDNEDYYMDVWGKGTTVTVSSASTKGP | YAASTLQSGVPSRFSG | SDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIALA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 181 | SEQ ID NO: 1018 | SEQ ID NO: 1017 | SEQ ID NO: 974 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWYQQKPGKAPKFLI | RGGGGSGGGGSGGGGSGGGGSGGGGSNWVNVI |
| | YYCTKDNEDYYMDVWGKGTTVTVSSASTKGP | YAASTLQSGVPSRFSG | SDLKKIEDLIQSMHIDATLYTESDVHPSCKVT |
| | SVFPLAPCSRSTSESTAALGCLVKDYFPEPV | SGYGTDFTLTISSLQP | AMKCFLLELQVISLESGDASIHDTVEDLIALA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTKTYTCNVDHKPSNTKVDKRVE | TFGQGTKVEIKRTVAA | FVHIVQMFINTSGGGGGKYGPPCPPCPAPEAA |
| | SKYGPPCPPCPAPEAAGGPSVFLFPPKPKDT | PSVFIFPPSDEQLKSG | GGPSVFLFPPKPKDTLMISRTPEVTCVVVDVS |
| | LMISRTPEVTCVVVDVSQEDPEVQFNWYVDG | TASVVCLLNNFYPREA | QEDPEVQFNWYVDGVEVHNAKTKPREEQFNST |
| | VEVHNAKTKPREEQFNSTYRVVSVLTVLHQD | KVQWKVDNALQSGNSQ | YRVVSVLTVLHQDWLNGKEYKCKVSNKGLPSS |
| | WLNGKEYKCKVSNKGLPSSIEKTISKAKGQP | ESVTEQDSKDSTYSLS | IEKTISKAKGQPREPQVYTLPPSQEEMTKNQV |
| | REPQVYTLPPSQEEMTKNQVSLSCAVKGFYP | STLTLSKADYEKHKVY | SLWCLVKGFYPSDIAVEWESNGQPENNYKTTP |
| | SDIAVEWESNGQPENNYKTTPPVLDSDGSFF | ACEVTHQGLSSPVTKS | PVLDSDGSFFLYSRLTVDKSRWQEGNVFSCSV |
| | LVSRLTVDKSRWQEGNVFSCSVMHEALHNRY | FNRGEC | MHEALHNHYTQKSLSLSLGK |
| | TQKSLSLSLGK | | |
| 182 | SEQ ID NO: 1018 | SEQ ID NO: 1017 | SEQ ID NO: 985 |
| | EVQLLESGGGLVQPGGSLRLSCAASGFTFST | AIQMTQSPSSLSASVG | ITCPPPMSVEHADIWVKSYSLYSRERYICNSG |
| | YAMSWVRQAPGKGLEWVSTISDNIGNTYYAD | DRVTITCRASQGIRND | FKRKAGTSSLTECVLNKATNVAHWTTPSLKCI |
| | SVKGRFTISRDNSKNTLYLQMNSLRAEDTAV | LGWYQQKPGKAPKELI | RGGGGSGGGGSGGGGSGGGGSGGGGSNWVNVI |
| | YYCTKDNEDYYMDVWGKGTTVTVSSASTKGP | YAASTLQSGVPSRFSG | GDLKKIEDLIQSMHIDATLYTESDVHPSCKVT |
| | SVFPLAPCSRSTSESTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYNYPR | NNSLSSNGNVTESGCKECEELEEKNIKEFLQS |
| | VTVPSSSLGTKTYTCNVDHKPSNTKVDKRVE | TFGQGTKVEIKRTVAA | FVHIVQMFINTSGGGGGKYGPPCPPCPAPEAA |
| | SKYGPPCPPCPAPEAAGGPSVFLFPPKPKDT | PSVFIFPPSDEQLKSG | GGPSVFLFPPKPKDTLMISRTPEVTCVVVDVS |
| | LMISRTPEVTCVVVDVSQEDPEVQFNWYVDG | TASVVCLLNNFYPREA | QEDPEVQFNWYVDGVEVHNAKTKPREEQFNST |
| | VEVHNAKTKPREEQFNSTYRVVSVLTVLHQD | KVQWKVDNALQSGNSQ | YRVVSVLTVLHQDWLNGKEYKCKVSNKGLPSS |
| | WLNGKEYKCKVSNKGLPSSIEKTISKAKGQP | ESVTEQDSKDSTYSLS | IEKTISKAKGQPREPQVYTLPPSQEEMTKNQV |
| | REPQVYTLPPSQEEMTKNQVSLSCAVKGFYP | STLTLSKADYEKHKVY | SLWCLVKGFYPSDIAVEWESNGQPENNYKTTP |
| | SDIAVEWESNGQPENNYKTTPPVLDSDGSFF | ACEVTHQGLSSPVTKS | PVLDSDGSFFLYSRLTVDKSRWQEGNVFSCSV |
| | LVSRLTVDKSRWQEGNVFSCSVMHEALHNRY | FNRGEC | MHEALHNHYTQKSLSLSLGK |
| | TQKSLSLSLGK | | |
| 183 | SEQ ID NO: 1019 | SEQ ID NO: 1020 | SEQ ID NO: 923 |
| | EVQLLESGGGLVQPGGSLRLSCSASGFTFSD | AIQMTQSPSSLSASVG | ITCPPPMSVEHADIWVKSYSLYSRERYICNSG |
| | YVMSWVRQAPGKGLDWVSSISGLTGNTYHAD | DRVTITCRASQGIRND | FKRKAGTSSLTECVLNKATNVAHWTTPSLKCI |
| | SVKGRFTISRDNSKNTLYLQMSSLRDEDTAV | LGWYQQKPGKAPKFLI | RGGGGSGGGGSGGGGSGGGGSGGGGSNWVNVI |
| | YYCAKDNEDYYMDVWGKGTTVTVSSASTKGP | SAASSLQSGVPSRFSG | SDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYTYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 184 | SEQ ID NO: 1019 | SEQ ID NO: 1020 | SEQ ID NO: 971 |
| | EVQLLESGGGLVQPGGSLRLSCSASGFTFSD | AIQMTQSPSSLSASVG | ITCPPPMSVEHADIWVKSYSLYSRERYICNSG |
| | YVMSWVRQAPGKGLDWVSSISGLTGNTYHAD | DRVTITCRASQGIRND | FKRKAGTSSLTECVLNKATNVAHWTTPSLKCI |
| | SVKGRFTISRDNSKNTLYLQMSSLRDEDTAV | LGWYQQKPGKAPKFLI | RGGGGSGGGGSGGGGSGGGGSGGGGSNWVNVI |
| | YYCAKDNEDYYMDVWGKGTTVTVSSASTKGP | SAASSLQSGVPSRFSG | GDLKKIEDLIQSMHIDATLYTESDVHPSCKVT |
| | SVEPLAPSSKSTSGGTAALGCLVKDYFPEPV | SGYGTDFTLTISSLQP | AMKCFLLELQVISLESGDASIHDTVEDLIILA |
| | TVSWNSGALTSGVHTFPAVLQSSGLYSLSSV | EDFATYYCLQDYTYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 185 | SEQ ID NO: 1019 | SEQ ID NO: 1020 | SEQ ID NO: 972 |
| | EVQLLESGGGLVQPGGSLRLSCSASGFTFSD | AIQMTQSPSSLSASVG | ITCPPPMSVEHADIWVKSYSLYSRERYICNSG |
| | YVMSWVRQAPGKGLDWVSSISGLTGNTYHAD | DRVTITCRASQGIRND | FKRKAGTSSLTECVLNKATNVAHWTTPSLKCI |
| | SVKGRFTISRDNSKNTLYLQMSSLRDEDTAV | LGWYQQKPGKAPKFLI | RGGGGSGGGGSGGGGSGGGGSGGGGSNWVNVI |
| | YYCAKDNEDYYMDVWGKGTTVTVSSASTKGP | SAASSLQSGVPSRFSG | SDLKKIEDLIQSMHIDATLYTESDVHPSCKVT |
| | SVFPLAPSSKSTSGGTAALGCLVKDYFPEPV | SGYGTDFTLTISSLQP | AMKCFLLELQVISLESGDASIHDTVEDLIALA |
| | TVSWNSGALTSGVHTFPAVLQSSGLYSLSSV | EDFATYYCLQDYTYPR | NNSLSSNGNVTESGCKECEELEEKNIKEFLQS |
| | VTVPSSSLGTQTYICNVNHKPSNTKVDKKVE | TFGQGTKVEIKRTVAA | FVHIVQMFINTSTSGGSDKTHTCPPCPAPEAA |
| | PKSCDKTHTCPPCPAPEAAGGPSVFLFPPKP | PSVFIFPPSDEQLKSG | GGPSVFLFPPKPKDTLMISRTPEVTCVVVDVS |
| | KDTLMISRTPEVTCVVVDVSHEDPEVKFNWY | TASVVCLLNNFYPREA | HEDPEVKFNWYVDGVEVHNAKTKPREEQYNST |
| | VDGVEVHNAKTKPREEQYNSTYRVVSVLTVL | KVQWKVDNALQSGNSQ | YRVVSVLTVLHQDWLNGKEYKCKVSNKALPAS |
| | HQDWLNGKEYKCKVSNKALPASIEKTISKAK | ESVTEQDSKDSTYSLS | IEKTISKAKGQPREPQVYTLPPSREEMTKNQV |
| | GQPREPQVYTLPPSREEMTKNQVSLSCAVKG | STLTLSKADYEKHKVY | SLWCLVKGFYPSDIAVEWESNGQPENNYKTTP |
| | FYPSDIAVEWESNGQPENNYKTTPPVLDSDG | ACEVTHQGLSSPVTKS | PVLDSDGSFFLYSKLTVDKSRWQQGNVFSCSV |
| | SFFLVSKLTVDKSRWQQGNVFSCSVMHEALH | FNRGEC | MHEALHNHYTQKSLSLSPGK |
| | NRYTQKSLSLSPGK | | |
| 186 | SEQ ID NO: 1021 | SEQ ID NO: 1020 | SEQ ID NO: 974 |
| | EVQLLESGGGLVQPGGSLRLSCSASGFTFSD | AIQMTQSPSSLSASVG | ITCPPPMSVEHADIWVKSYSLYSRERYICNSG |
| | YVMSWVRQAPGKGLDWVSSISGLTGNTYHAD | DRVTITCRASQGIRND | FKRKAGTSSLTECVLNKATNVAHWTTPSLKCI |
| | SVKGRFTISRDNSKNTLYLQMSSLRDEDTAV | LGWYQQKPGKAPKFLI | RGGGGSGGGGSGGGGSGGGGSGGGGSNWVNVI |
| | YYCAKDNEDYYMDVWGKGTTVTVSSASTKGP | SAASSLQSGVPSRFSG | SDLKKIEDLIQSMHIDATLYTESDVHPSCKVT |
| | SVFPLAPCSRSTSESTAALGCLVKDYFPEPV | SGYGTDFTLTISSLQP | AMKCFLLELQVISLESGDASIHDTVEDLIALA |
| | TVSWNSGALTSGVHTFPAVLQSSGLYSLSSV | EDFATYYCLQDYTYPR | NNSLSSNGNVTESGCKECEELEEKNIKEFLQS |
| | VTVPSSSLGTKTYTCNVDHKPSNTKVDKRVE | TFGQGTKVEIKRTVAA | FVHIVQMFINTSGGGGGKYGPPCPPCPAPEAA |
| | SKYGPPCPPCPAPEAAGGPSVFLFPPKPKDT | PSVFIFPPSDEQLKSG | GGPSVFLFPPKPKDTLMISRTPEVTCVVVDVS |
| | LMISRTPEVTCVVVDVSQEDPEVQFNWYVDG | TASVVCLLNNFYPREA | QEDPEVQFNWYVDGVEVHNAKTKPREEQFNST |
| | VEVHNAKTKPREEQFNSTYRVVSVLTVLHQD | KVQWKVDNALQSGNSQ | YRVVSVLTVLHQDWLNGKEYKCKVSNKGLPSS |
| | WLNGKEYKCKVSNKGLPSSIEKTISKAKGQP | ESVTEQDSKDSTYSLS | IEKTISKAKGQPREPQVYTLPPSQEEMTKNQV |
| | REPQVYTLPPSQEEMTKNQVSLSCAVKGFYP | STLTLSKADYEKHKVY | SLWCLVKGFYPSDIAVEWESNGQPENNYKTTP |
| | SDIAVEWESNGQPENNYKTTPPVLDSDGSFF | ACEVTHQGLSSPVTKS | PVLDSDGSFFLYSRLTVDKSRWQEGNVFSCSV |
| | LVSRLTVDKSRWQEGNVFSCSVMHEALHNRY | FNRGEC | MHEALHNHYTQKSLSLSLGK |
| | TQKSLSLSLGK | | |
| 187 | SEQ ID NO: 1022 | SEQ ID NO: 1023 | SEQ ID NO: 923 |
| | EVQLLESGGGLVQPGGSLRLSCAASGFTFST | AIQMTQSPSSLSASVG | ITCPPPMSVEHADIWVKSYSLYSRERYICNSG |
| | NAMSWVRQAPGKGLEWVSGISGSNGNTYYAD | DRVTITCRASHVIRND | FKRKAGTSSLTECVLNKATNVAHWTTPSLKCI |
| | SVKGRFIISRDISKNTLYLEMNSLRAEDTAV | LGWYQQKPGKAPKLLI | RGGGGGGGGSGGGGSGGGGSGGGGSNWVNVI |
| | YYCAKERDNWNGFDYWGQGTLVTVSSASTKG | YAASSLQSGVPSRFSG | SDLKKIEDLIQSMHIDATLYTESDVHPSCKVT |
| | PSVFPLAPSSKSTSGGTAALGCLVKDYFPEP | SGSGTDFTLTISSLQP | AMKCFLLELQVISLESGDASIHDTVEDLIILA |
| | VTVSWNSGALTSGVHTFPAVLQSSGLYSLSS | EDFATYYCLQDYNYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 188 | SEQ ID NO: 1024 | SEQ ID NO: 1025 | SEQ ID NO: 923 |
| | QVQLVQSGAEVKKPGASVKVSCKVSGHTLTE | ETTLTQSPAFMSATPG | ITCPPPMSVEHADIWVKSYSLYSRERYICNSG |
| | LSMHWVRQAPGKGLEWMGIFDPRDGQIIYAE | DKVNISCKASQDIDDD | FKRKAGTSSLTECVLNKATNVAHWTTPSLKCI |
| | KFQGRVTVTEDTSTDTAYMELSSLREDDTAV | VNWYQQKPGEAAIFII | RGGGGSGGGGSGGGGSGGGGSGGGGSNWVNVI |
| | YYCATGGNEDYYFYYMDVWGKGTTVTVSSAS | QEATTLVPGIPPRFSG | SDLKKIEDLIQSMHIDATLYTESDVHPSCKVT |
| | TKGPSVFPLAPSSKSTSGGTAALGCLVKDYF | SGYGTDETLTINNIES | AMKCFLLELQVISLESGDASIHDTVEDLIILA |
| | PEPVTVSWNSGALTSGVHTFPAVLQSSGLYS | EDAAYYFCLQHDHFPY | NNSLSSNGNVTESGCKECEELEEKNIKEFLQS |
| | LSSVVTVPSSSLGTQTYICNVNHKPSNTKVD | TFGQGTKLEIKRTVAA | FVHIVQMFINTSTSGGSDKTHTCPPCPAPEAA |
| | KKVEPKSCDKTHTCPPCPAPEAAGGPSVFLF | PSVFIFPPSDEQLKSG | GGPSVFLFPPKPKDTLMISRTPEVTCVVVDVS |
| | PPKPKDTLMISRTPEVTCVVVDVSHEDPEVK | TASVVCLLNNFYPREA | HEDPEVKFNWYVDGVEVHNAKTKPREEQYNST |
| | FNWYVDGVEVHNAKTKPREEQYNSTYRVVSV | KVQWKVDNALQSGNSQ | YRVVSVLTVLHQDWLNGKEYKCKVSNKALPAS |
| | LTVLHQDWLNGKEYKCKVSNKALPASIEKTI | ESVTEQDSKDSTYSLS | IEKTISKAKGQPREPQVYTLPPSREEMTKNQV |
| | SKAKGQPREPQVYTLPPSREEMTKNQVSLSC | STLTLSKADYEKHKVY | SLWCLVKGFYPSDIAVEWESNGQPENNYKTTP |
| | AVKGFYPSDIAVEWESNGQPENNYKTTPPVL | ACEVTHQGLSSPVTKS | PVLDSDGSFFLYSKLTVDKSRWQQGNVFSCSV |
| | DSDGSFFLVSKLTVDKSRWQQGNVFSCSVMH | FNRGEC | MHEALHNHYTQKSLSLSPGK |
| | EALHNRYTQKSLSLSPGK | | |
| 189 | SEQ ID NO: 1011 | SEQ ID NO: 1014 | SEQ ID NO: 1026 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSLGPVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 190 | SEQ ID NO: 1027 | SEQ ID NO: 1014 | SEQ ID NO: 1028 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGAPSVFLFPPK | PSVFIFPPSDEQLKSG | GAPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAP |
| | LHQDWLNGKEYKCKVSNKALPAPIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 191 | SEQ ID NO: 1027 | SEQ ID NO: 1014 | SEQ ID NO: 1029 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSLGPVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGAPSVFLFPPK | PSVFIFPPSDEQLKSG | GAPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAP |
| | LHQDWLNGKEYKCKVSNKALPAPIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 192 | SEQ ID NO: 1011 | SEQ ID NO: 1014 | SEQ ID NO: 1030 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 193 | SEQ ID NO: 1011 | SEQ ID NO: 1014 | SEQ ID NO: 1031 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSNGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 194 | SEQ ID NO: 1011 | SEQ ID NO: 1014 | SEQ ID NO: 1032 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDETLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSLGNVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 220 | SEQ ID NO: 1011 | SEQ ID NO: 1014 | SEQ ID NO: 1077 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGGVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 195 | SEQ ID NO: 1011 | SEQ ID NO: 1014 | SEQ ID NO: 1033 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGPVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 196 | SEQ ID NO: 1011 | SEQ ID NO: 1014 | SEQ ID NO: 1034 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 197 | SEQ ID NO: 1011 | SEQ ID NO: 1014 | SEQ ID NO: 1035 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 198 | SEQ ID NO: 1011 | SEQ ID NO: 1014 | SEQ ID NO: 1036 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGNVTESGCKECEELEEKNIKEFLQS |
| | VVTVPSSSLGTQTYICNVNHKPSNTKVDKKV | TFGQGTKLEIKRTVAA | FVHIVQMFISTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 199 | SEQ ID NO: 1011 | SEQ ID NO: 1014 | SEQ ID NO: 1037 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSNGPVTESGCKECEELEEKNIKEFLQS |
| | VVTVPSSSLGTQTYICNVNHKPSNTKVDKKV | TFGQGTKLEIKRTVAA | FVHIVQMFINTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLIV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 200 | SEQ ID NO: 1011 | SEQ ID NO: 1014 | SEQ ID NO: 1038 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSNGNVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 201 | SEQ ID NO: 1011 | SEQ ID NO: 1014 | SEQ ID NO: 1039 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | NNSLSSNGPVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 202 | SEQ ID NO: 1011 | SEQ ID NO: 1014 | SEQ ID NO: 1040 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | QNSLSSNGQVTESGCKECEELEEKNIKEFLQS |
| | VVTVPSSSLGTQTYICNVNHKPSNTKVDKKV | TFGQGTKLEIKRTVAA | FVHIVQMFIQTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 203 | SEQ ID NO: 1011 | SEQ ID NO: 1014 | SEQ ID NO: 1041 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ANSLSSNGAVTESGCKECEELEEKNIKEFLQS |
| | VVTVPSSSLGTQTYICNVNHKPSNTKVDKKV | TFGQGTKLEIKRTVAA | FVHIVQMFIATSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 204 | SEQ ID NO: 1011 | SEQ ID NO: 1014 | SEQ ID NO: 1042 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | GNSLSSNGGVTESGCKECEELEEKNIKEFLQS |
| | VVTVPSSSLGTQTYICNVNHKPSNTKVDKKV | TFGQGTKLEIKRTVAA | FVHIVQMFIGTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 205 | SEQ ID NO: 1011 | SEQ ID NO: 1014 | SEQ ID NO: 1043 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | SNSLSSNGLVTESGCKECEELEEKNIKEFLQS |
| | VVTVPSSSLGTQTYICNVNHKPSNTKVDKKV | TFGQGTKLEIKRTVAA | FVHIVQMFIETSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 206 | SEQ ID NO: 1011 | SEQ ID NO: 1014 | SEQ ID NO: 1044 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSNGPVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 207 | SEQ ID NO: 1011 | SEQ ID NO: 1014 | SEQ ID NO: 1045 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSNGPVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLIV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 208 | SEQ ID NO: 1011 | SEQ ID NO: 1014 | SEQ ID NO: 1046 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | LNSLSSNGPVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 209 | SEQ ID NO: 1011 | SEQ ID NO: 1014 | SEQ ID NO: 1047 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | KNSLSSNGPVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 210 | SEQ ID NO: 1011 | SEQ ID NO: 1014 | SEQ ID NO: 1048 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSNGPVTESGCKECEELEEKNIKEFLQS |
| | VVTVPSSSLGTQTYICNVNHKPSNTKVDKKV | TFGQGTKLEIKRTVAA | FVHIVQMFISTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 211 | SEQ ID NO: 1011 | SEQ ID NO: 1014 | SEQ ID NO: 1049 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSNGPVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 212 | SEQ ID NO: 1011 | SEQ ID NO: 1014 | SEQ ID NO: 1050 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | QNSLSSLGQVTESGCKECEELEEKNIKEFLQS |
| | VVTVPSSSLGTQTYICNVNHKPSNTKVDKKV | TFGQGTKLEIKRTVAA | FVHIVQMFIQTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 213 | SEQ ID NO: 1011 | SEQ ID NO: 1014 | SEQ ID NO: 1051 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ANSLSSLGAVTESGCKECEELEEKNIKEFLQS |
| | VVTVPSSSLGTQTYICNVNHKPSNTKVDKKV | TFGQGTKLEIKRTVAA | FVHIVQMFIATSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 214 | SEQ ID NO: 1011 | SEQ ID NO: 1014 | SEQ ID NO: 1052 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | GNSLSSLGGVTESGCKECEELEEKNIKEFLQS |
| | VVTVPSSSLGTQTYICNVNHKPSNTKVDKKV | TFGQGTKLEIKRTVAA | FVHIVQMFIGTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 215 | SEQ ID NO: 1011 | SEQ ID NO: 1014 | SEQ ID NO: 1053 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | SNSLSSLGLVTESGCKECEELEEKNIKEFLQS |
| | VVTVPSSSLGTQTYICNVNHKPSNTKVDKKV | TFGQGTKLEIKRTVAA | FVHIVQMFIETSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 216 | SEQ ID NO: 1011 | SEQ ID NO: 1014 | SEQ ID NO: 1054 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSLGPVTESGCKECEELEEKNIKEFLQS |
| | VVTVPSSSLGTQTYICNVNHKPSNTKVDKKV | TFGQGTKLEIKRTVAA | FVHIVQMFIKTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 217 | SEQ ID NO: 1011 | SEQ ID NO: 1014 | SEQ ID NO: 1055 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSLGPVTESGCKECEELEEKNIKEFLQS |
| | VVTVPSSSLGTQTYICNVNHKPSNTKVDKKV | TFGQGTKLEIKRTVAA | FVHIVQMFISTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 218 | SEQ ID NO: 1011 | SEQ ID NO: 1014 | SEQ ID NO: 1056 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | GDLKKIEDLIQSMHIDATLYTESDVHPSCKVT |
| | PSVFPLAPSSKSTSGGTAALGCLVKDYFPEP | SRSGTDFTLTISSLQP | AMKCFLLELQVISLESGDASIHDTVEDLIILA |
| | VTVSWNSGALTSGVHTFPAVLQSSGLYSLSS | EDFATYYCLQDYTYPY | ENSLSSLGPVTESGCKECEELEEKNIKEFLQS |
| | VVTVPSSSLGTQTYICNVNHKPSNTKVDKKV | TFGQGTKLEIKRTVAA | FVHIVQMFIDTSTSGGSDKTHTCPPCPAPEAA |
| | EPKSCDKTHTCPPCPAPEAAGGPSVFLFPPK | PSVFIFPPSDEQLKSG | GGPSVFLFPPKPKDTLMISRTPEVTCVVVDVS |
| | PKDTLMISRTPEVTCVVVDVSHEDPEVKFNW | TASVVCLLNNFYPREA | HEDPEVKFNWYVDGVEVHNAKTKPREEQYNST |
| | YVDGVEVHNAKTKPREEQYNSTYRVVSVLTV | KVQWKVDNALQSGNSQ | YRVVSVLTVLHQDWLNGKEYKCKVSNKALPAS |
| | LHQDWLNGKEYKCKVSNKALPASIEKTISKA | ESVTEQDSKDSTYSLS | IEKTISKAKGQPREPQVYTLPPSREEMTKNQV |
| | KGQPREPQVYTLPPSREEMTKNQVSLSCAVK | STLTLSKADYEKHKVY | SLWCLVKGFYPSDIAVEWESNGQPENNYKTTP |
| | GFYPSDIAVEWESNGQPENNYKTTPPVLDSD | ACEVTHQGLSSPVTKS | PVLDSDGSFFLYSKLTVDKSRWQQGNVFSCSV |
| | GSFFLVSKLTVDKSRWQQGNVFSCSVMHEAL | FNRGEC | MHEALHNHYTQKSLSLSPGK |
| | HNRYTQKSLSLSPGK | | |
| 219 | SEQ ID NO: 1011 | SEQ ID NO: 1014 | SEQ ID NO: 1057 |
| | EVELLESGGGLVQPGGSLRLSCAASGLTFST | AIQMTQSPSSLSASVG | ITCPPPMSVEHADIWVKSYSLYSRERYICNSG |
| | FAMSWVRQAPGKGLEWVSGISGSGENTYYAD | DRVTITCRASHGIRND | FKRKAGTSSLTECVLNKATNVAHWTTPSLKCI |
| | SVKGRFTITSDNSKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | X₁DLKKIEDLIQSMHIDATLYTESDVHPSCKV |
| | PSVFPLAPSSKSTSGGTAALGCLVKDYFPEP | SRSGTDFTLTISSLQP | TAMKCFLLELQVISLESGDASIHDTVEX₂LII |
| | VTVSWNSGALTSGVHTFPAVLQSSGLYSLSS | EDFATYYCLQDYTYPY | LAX₃NSLSSX₄GX₅VTESGCKECEELEEKNIKE |
| | VVTVPSSSLGTQTYICNVNHKPSNTKVDKKV | TFGQGTKLEIKRTVAA | FLQSFVHIVQMFIX₆TSTSGGSDKTHTCPPCP |
| | EPKSCDKTHTCPPCPAPEAAGGPSVFLFPPK | PSVFIFPPSDEQLKSG | APEAAGGPSVFLFPPKPKDTLMISRTPEVTCV |
| | PKDTLMISRTPEVTCVVVDVSHEDPEVKFNW | TASVVCLLNNFYPREA | VVDVSHEDPEVKFNWYVDGVEVHNAKTKPREE |
| | YVDGVEVHNAKTKPREEQYNSTYRVVSVLTV | KVQWKVDNALQSGNSQ | QYNSTYRVVSVLTVLHQDWLNGKEYKCKVSNK |
| | LHQDWLNGKEYKCKVSNKALPASIEKTISKA | ESVTEQDSKDSTYSLS | ALPASIEKTISKAKGQPREPQVYTLPPSREEM |
| | KGQPREPQVYTLPPSREEMTKNQVSLSCAVK | STLTLSKADYEKHKVY | TKNQVSLWCLVKGFYPSDIAVEWESNGQPENN |
| | GFYPSDIAVEWESNGQPENNYKTTPPVLDSD | ACEVTHQGLSSPVTKS | YKTTPPVLDSDGSFFLYSKLTVDKSRWQQGNV |
| | GSFFLVSKLTVDKSRWQQGNVFSCSVMHEAL | FNRGEC | FSCSVMHEALHNHYTQKSLSLSPGK |
| | HNRYTQKSLSLSPGK | | X₁ is G or S |
| | | | X₂ is D or N |
| | | | X₃ is A, E, G, K, L, N, Q or S |
| | | | X₄ is N or L |
| | | | X₅ is A, G, L, N, P, Q or S |
| | | | X₆ is A, D, E, G, K, Q or S |

Binds to CD4 D3; has 4 polypeptide chains; 2 HC and 2 LC

| Ab | | CD4-binding | CD4-binding domain HC-IL-15Rα |
|---|---|---|---|
| Name | CD4-binding domain HC | domain LC x 2 | Sushi domain-IL-15-Fc fusion |
| 222 | SEQ ID NO: 1013 | SEQ ID NO: 1014 | SEQ ID NO: 1061 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVF |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISGLKKIEDLIQSMHI |
| | | | DATLYTESDVHPSCKVTAMKCFLLELQVISLE |
| | | | SGDASIHDTVENLIILANNSLSSNGNVTESGC |
| | | | KECEELEEKNIKEFLQSFVHIVQMFINTS |
| 223 | SEQ ID NO: 1013 | SEQ ID NO: 1014 | SEQ ID NO: 1062 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVF |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISDLKKIEDLIQSMHI |
| | | | DATLYTESDVHPSCKVTAMKCFLLELQVISLE |
| | | | SGDASIHDTVEDLIALANNSLSSNGNVTESGC |
| | | | KECEELEEKNIKEFLQSFVHIVQMFINTS |
| 224 | SEQ ID NO: 1013 | SEQ ID NO: 1014 | SEQ ID NO:1063 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVE |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVIGDLKKIEDLIQSMHI |
| | | | DATLYTESDVHPSCKVTAMKCFLLELQVISLE |
| | | | SGDASIHDTVEDLIALANNSLSSNGNVTESGC |
| | | | KECEELEEKNIKEFLQSFVHIVQMFINTS |
| 225 | SEQ ID NO: 1013 | SEQ ID NO: 1014 | SEQ ID NO: 1064 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVE |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGGGGGSGGGG |
| | | | SGGGGSGGGGSNWVNVIGDLKKIEDLIQSMHI |
| | | | DATLYTESDVHPSCKVTAMKCFLLELQVISLE |
| | | | SGDASIHDTVEALIALANNSLSSNGNVTESGC |
| | | | KECEELEEKNIKEFLQSFVHIVQMFINTS |
| 226 | SEQ ID NO: 1013 | SEQ ID NO: 1014 | SEQ ID NO: 1065 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVE |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISGLKKIEDLIQSMHI |
| | | | DATLYTESDVHPSCKVTAMKCFLLELQVISLE |
| | | | SGDASIHDTVEDLIILANNSLSSNGNVTESGC |
| | | | KECEELEEKNIKEFLQSFVHIVQMFINTS |
| 227 | SEQ ID NO: 1013 | SEQ ID NO: 1014 | SEQ ID NO: 1066 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVF |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISDLKKIEDLIQSMHI |
| | | | DATLYTESDVHPSCKVTAMKCFLLELQVISLE |
| | | | SGDASIHDTVEALIALANNSLSSNGNVTESGC |
| | | | KECEELEEKNIKEFLQSFVHIVQMFINTS |
| 228 | SEQ ID NO: 1013 | SEQ ID NO: 1014 | SEQ ID NO : 1067 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVE |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISDLKKIEDLIQSMHI |
| | | | DATLYTESDVHPSCKVTAMKCFLLELQVISLE |
| | | | SGDASIHDTVENLIILANNSLSSNGNVTESGC |
| | | | KECEELEEKNIKEFLQSFVHIVEMFINTS |
| 229 | SEQ ID NO: 1013 | SEQ ID NO: 1014 | SEQ ID NO: 1068 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVE |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISDLKKIEDLIQSMHI |
| | | | DATLYTESDVHPSCKVTAMKCELLELQVISLE |
| | | | SGDASIHNTVEDLIALANNSLSSNGNVTESGC |
| | | | KECEELEEKNIKEFLQSFVHIVQMFINTS |
| 230 | SEQ ID NO: 1013 | SEQ ID NO: 1014 | SEQ ID NO: 1069 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVF |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISGLKKIEDLIQSMHI |
| | | | DATLYTESDVHPSCKVTAMKCFLLELQVISLE |
| | | | SGDASIHDTVQNLIILANNSLSSNGNVTESGC |
| | | | KECEELEEKNIKEFLQSFVHIVQMFINTS |
| 231 | SEQ ID NO: 1013 | SEQ ID NO: 1014 | SEQ ID NO: 1070 |
| | EVELLESGGGLVQPGGSLRLSCAASGLTFST | AIQMTQSPSSLSASVG | EVELLESGGGLVQPGGSLRLSCAASGLTFSTF |
| | FAMSWVRQAPGKGLEWVSGISGSGENTYYAD | DRVTITCRASHGIRND | AMSWVRQAPGKGLEWVSGISGSGENTYYADSV |
| | SVKGRFTITSDNSKNTLYLQMNSLRAEDTAV | LGWYQQKPGKAPKVLI | KGRFTITSDNSKNTLYLQMNSLRAEDTAVYYC |
| | YYCAKEGYNWNYMDVWGKGTTVTVSSASTKG | YEASSLQSGVPSRFSG | AKEGYNWNYMDVWGKGTTVTVSSASTKGPSVF |
| | PSVFPLAPCSRSTSESTAALGCLVKDYFPEP | SRSGTDFTLTISSLQP | PLAPCSRSTSESTAALGCLVKDYFPEPVTVSW |
| | VTVSWNSGALTSGVHTFPAVLQSSGLYSLSS | EDFATYYCLQDYTYPY | NSGALTSGVHTFPAVLQSSGLYSLSSVVTVPS |
| | VVTVPSSSLGTKTYTCNVDHKPSNTKVDKRV | TFGQGTKLEIKRTVAA | SSLGTKTYTCNVDHKPSNTKVDKRVESKYGPP |
| | ESKYGPPCPPCPAPEAAGGPSVFLFPPKPKD | PSVFIFPPSDEQLKSG | CPPCPAPEAAGGPSVFLFPPKPKDTLMISRTP |
| | TLMISRTPEVTCVVVDVSQEDPEVQFNWYVD | TASVVCLLNNFYPREA | EVTCVVVDVSQEDPEVQFNWYVDGVEVHNAKT |
| | GVEVHNAKTKPREEQFNSTYRVVSVLTVLHQ | KVQWKVDNALQSGNSQ | KPREEQFNSTYRVVSVLTVLHQDWLNGKEYKC |
| | DWLNGKEYKCKVSNKGLPSSIEKTISKAKGQ | ESVTEQDSKDSTYSLS | KVSNKGLPSSIEKTISKAKGQPREPQVYTLPP |
| | PREPQVYTLPPSQEEMTKNQVSLSCAVKGFY | STLTLSKADYEKHKVY | SQEEMTKNQVSLWCLVKGFYPSDIAVEWESNG |
| | PSDIAVEWESNGQPENNYKTTPPVLDSDGSF | ACEVTHQGLSSPVTKS | QPENNYKTTPPVLDSDGSFFLYSRLTVDKSRW |
| | FLVSRLTVDKSRWQEGNVFSCSVMHEALHNR | FNRGEC | QEGNVFSCSVMHEALHNHYTQKSLSLSLGGGG |
| | YTQKSLSLSLGK | | GSGGGGSGGGGSGGGGSITCPPPMSVEHADIW |
| | | | VKSYSLYSRERYICNSGFKRKAGTSSLTECVL |
| | | | NKATNVAHWTTPSLKCIRGGGGSGGGGSGGGG |
| | | | SGGGGSGGGGSNWVNVISDLKKIEDLIQSMHI |
| | | | DATLYTESDVHPSCKVTAMKCFLLELQVISLE |
| | | | SGDASIHDTVQNLIILANNSLSSNGNVTESGC |
| | | | KECEELEEKNIKEFLQSFVHIVEMFINTS |
| 232 | SEQ ID NO: 1071 | SEQ ID NO: 1017 | SEQ ID NO: 1072 |
| | EVQLLESGGGLVQPGGSLRLSCAASGFTFST | AIQMTQSPSSLSASVG | EVQLLESGGGLVQPGGSLRLSCAASGFTFSTY |
| | YAMSWVRQAPGKGLEWVSTISDNIGNTYYAD | DRVTITCRASQGIRND | AMSWVRQAPGKGLEWVSTISDNIGNTYYADSV |
| | SVKGRFTISRDNSKNTLYLQMNSLRAEDTAV | LGWYQQKPGKAPKFLI | KGRFTISRDNSKNTLYLQMNSLRAEDTAVYYC |
| | YYCTKDNEDYYMDVWGKGTTVTVSSASTKGP | YAASTLQSGVPSRFSG | TKDNEDYYMDVWGKGTTVTVSSASTKGPSVFP |
| | SVFPLAPCSRSTSESTAALGCLVKDYFPEPV | SGYGTDFTLTISSLQP | LAPCSRSTSESTAALGCLVKDYFPEPVTVSWN |
| | TVSWNSGALTSGVHTFPAVLQSSGLYSLSSV | EDFATYYCLQDYNYPR | SGALTSGVHTFPAVLQSSGLYSLSSVVTVPSS |
| | VTVPSSSLGTKTYTCNVDHKPSNTKVDKRVE | TFGQGTKVEIKRTVAA | SLGTKTYTCNVDHKPSNTKVDKRVESKYGPPC |
| | SKYGPPCPPCPAPEAAGGPSVFLFPPKPKDT | PSVFIFPPSDEQLKSG | PPCPAPEAAGGPSVFLFPPKPKDTLMISRTPE |
| | LMISRTPEVTCVVVDVSQEDPEVQFNWYVDG | TASVVCLLNNFYPREA | VTCVVVDVSQEDPEVQFNWYVDGVEVHNAKTK |
| | VEVHNAKTKPREEQFNSTYRVVSVLTVLHQD | KVQWKVDNALQSGNSQ | PREEQFNSTYRVVSVLTVLHQDWLNGKEYKCK |
| | WLNGKEYKCKVSNKGLPSSIEKTISKAKGQP | ESVTEQDSKDSTYSLS | VSNKGLPSSIEKTISKAKGQPREPQVYTLPPS |
| | REPQVYTLPPSQEEMTKNQVSLSCAVKGFYP | STLTLSKADYEKHKVY | QEEMTKNQVSLWCLVKGFYPSDIAVEWESNGQ |
| | SDIAVEWESNGQPENNYKTTPPVLDSDGSFF | ACEVTHQGLSSPVTKS | PENNYKTTPPVLDSDGSFFLYSRLTVDKSRWQ |
| | LVSRLTVDKSRWQEGNVFSCSVMHEALHNRY | FNRGEC | EGNVFSCSVMHEALHNHYTQKSLSLSLGGGGG |
| | TQKSLSLSL | | SGGGGSGGGGSGGGGSITCPPPMSVEHADIWV |
| | | | KSYSLYSRERYICNSGFKRKAGTSSLTECVLN |
| | | | KATNVAHWTTPSLKCIRGGGGSGGGGSGGGGS |
| | | | GGGGSGGGGSNWVNVISDLKKIEDLIQSMHID |
| | | | ATLYTESDVHPSCKVTAMKCFLLELQVISLES |
| | | | GDASIHDTVEDLIALANNSLSSNGNVTESGCK |
| | | | ECEELEEKNIKEFLQSFVHIVQMFINTS |
| 233 | SEQ ID NO: 1071 | SEQ ID NO: 1017 | SEQ ID NO: 1073 |
| | EVQLLESGGGLVQPGGSLRLSCAASGFTFST | AIQMTQSPSSLSASVG | EVQLLESGGGLVQPGGSLRLSCAASGFTFSTY |
| | YAMSWVRQAPGKGLEWVSTISDNIGNTYYAD | DRVTITCRASQGIRND | AMSWVRQAPGKGLEWVSTISDNIGNTYYADSV |
| | SVKGRFTISRDNSKNTLYLQMNSLRAEDTAV | LGWYQQKPGKAPKFLI | KGRFTISRDNSKNTLYLQMNSLRAEDTAVYYC |
| | YYCTKDNEDYYMDVWGKGTTVTVSSASTKGP | YAASTLQSGVPSRFSG | TKDNEDYYMDVWGKGTTVTVSSASTKGPSVFP |
| | SVFPLAPCSRSTSESTAALGCLVKDYFPEPV | SGYGTDFTLTISSLQP | LAPCSRSTSESTAALGCLVKDYFPEPVTVSWN |
| | TVSWNSGALTSGVHTFPAVLQSSGLYSLSSV | EDFATYYCLQDYNYPR | SGALTSGVHTFPAVLQSSGLYSLSSVVTVPSS |
| | VTVPSSSLGTKTYTCNVDHKPSNTKVDKRVE | TFGQGTKVEIKRTVAA | SLGTKTYTCNVDHKPSNTKVDKRVESKYGPPC |
| | SKYGPPCPPCPAPEAAGGPSVFLFPPKPKDT | PSVFIFPPSDEQLKSG | PPCPAPEAAGGPSVFLFPPKPKDTLMISRTPE |
| | LMISRTPEVTCVVVDVSQEDPEVQFNWYVDG | TASVVCLLNNFYPREA | VTCVVVDVSQEDPEVQFNWYVDGVEVHNAKTK |
| | VEVHNAKTKPREEQFNSTYRVVSVLTVLHQD | KVQWKVDNALQSGNSQ | PREEQFNSTYRVVSVLTVLHQDWLNGKEYKCK |
| | WLNGKEYKCKVSNKGLPSSIEKTISKAKGQP | ESVTEQDSKDSTYSLS | VSNKGLPSSIEKTISKAKGQPREPQVYTLPPS |
| | REPQVYTLPPSQEEMTKNQVSLSCAVKGFYP | STLTLSKADYEKHKVY | QEEMTKNQVSLWCLVKGFYPSDIAVEWESNGQ |
| | SDIAVEWESNGQPENNYKTTPPVLDSDGSFF | ACEVTHQGLSSPVTKS | PENNYKTTPPVLDSDGSFFLYSRLTVDKSRWQ |
| | LVSRLTVDKSRWQEGNVFSCSVMHEALHNRY | FNRGEC | EGNVFSCSVMHEALHNHYTQKSLSLSLGGGGG |
| | TQKSLSLSL | | SGGGGSGGGGSGGGGSITCPPPMSVEHADIWV |
| | | | KSYSLYSRERYICNSGFKRKAGTSSLTECVLN |
| | | | KATNVAHWTTPSLKCIRGGGGSGGGGSGGGGS |
| | | | GGGGSGGGGSNWVNVIGDLKKIEDLIQSMHID |
| | | | ATLYTESDVHPSCKVTAMKCFLLELQVISLES |
| | | | GDASIHDTVEDLIALANNSLSSNGNVTESGCK |
| | | | ECEELEEKNIKEFLQSFVHIVQMFINTS |

TABLE H
amino acid sequences of illustrative simianized CD4-targeted IL-15 molecules

| | | | |
|---|---|---|---|
| Ab | | CD4-binding | IL-15Rα Sushi domain-IL-15-Fc |
| Name | CD4-binding domain HC | domain LC | fusion protein |

Binds to CD4 D1

| | | | |
|---|---|---|---|
| 247 | SEQ ID NO: 1101 | SEQ ID NO: 1102 | SEQ ID NO: 1103 |
| | EVQLVESGGGLAKPGGSLRLSCAASGFSFSN | DIQMTQSPSSLSASVG | ITCPPPVSVEHADIRVKSYSLYSRERYICNSG |
| | YAMSWVRQAPGKGLEWVASINDGGSTFYADS | DRVTITCRASQDINKY | FKRKAGTSSLTECVLNKATNIAHWTTPSLKCI |
| | VKGRFTISRENAKNTLYLQMNSLRAEDTAVY | IAWYQQKPGKGPKLLI | RGGGGSGGGGSGGGGSGGGGSGGGGSNWVNVI |
| | YCSRHYGGSYDPMDYWGQGVLVTVSSASTKG | HYTSTLHPGVPSRFSG | SDLKKIEDLIQSMHIDATLYTESDVHPSCKVT |
| | PSVFPLAPSSRSTSESTAALGCLVKDYFPEP | SGSGTDYTLTISSLQP | AMKCFLLELQVISHESGDTDIHDTVEDLIILA |
| | VTVSWNSGSLTSGVHTFPAVLQSSGLYSLSS | EDFATYYCLQYDNPLY | NNILSSNGNITESGCKECEELEEKNIKEFLQS |
| | VVTVPSSSLGTQTYVCNVNHKPSNTKVDKRV | TFGGGTKVEIKRAVAA | FVHIVQMFINTSTSGGGSKPPTCPPCPAPEAA |
| | EIKTCGGGSKPPTCPPCPAPEAAGGPSVFLF | PSVFIFPPSEDQVKSG | GGPSVFLFPPKPKDTLMISRTPEVTCVVVDVS |
| | PPKPKDTLMISRTPEVTCVVVDVSQEDPDVK | TVSVVCLLNNFYPREA | QEDPDVKFNWYVNGAEVHHAQTKPRETQYNST |
| | FNWYVNGAEVHHAQTKPRETQYNSTYRVVSV | SVKWKVDGVLKTGNSQ | YRVVSVLTVTHQDWLNGKEYTCKVSNKALPAS |
| | LTVTHQDWLNGKEYTCKVSNKALPASIQKTI | ESVTEQDSKDNTYSLS | IQKTISKDKGQPREPQVYTLPPSREELTKNQV |
| | SKDKGQPREPQVYTLPPSREELTKNQVSLSC | STLTLSSTDYQSHNVY | SLWCLVKGFYPSDIVVEWESSGQPENTYKTTP |
| | AVKGFYPSDIVVEWESSGQPENTYKTTPPVL | ACEVTHQGLSSPVTKS | PVLDSDGSYFLYSKLTVDKSRWQQGNVFSCSV |
| | DSDGSYFLVSKLTVDKSRWQQGNVFSCSVMH | FNRGEC | MHEALHNHYTQKSLSVSPGK |
| | EALHNRYTQKSLSVSPGK | | |

Binds to CD4 D3

| | | | |
|---|---|---|---|
| 221 | SEQ ID NO: 1058 | SEQ ID NO: 1059 | SEQ ID NO: 1060 |
| | EVQLVESGGGLVQPGGSLRLSCVASGLTFST | DIQMTQSPSSLSASVG | ITCPPPVSVEHADIRVKSYSLYSRERYICNSG |
| | FAMSWVRQAPGKGLEWVSGISGSGENTYYAD | DRVTITCRASHGIRND | FKRKAGTSSLTECVLNKATNIAHWTTPSLKCI |
| | SVKGRFTISSDNAKNTLYLQMNSLRAEDTAV | LGWYQQKPGKAPKVLI | RGGGGSGGGGSGGGGSGGGGSGGGGSNWVNVI |
| | YYCAKEGYNWNYMDVWGKGTTVTVSSASTKG | YEASSLQSGVPSRFSG | SDLKKIEDLIQSMHIDATLYTESDVHPSCKVT |
| | PSVFPLAPSSRSTSESTAALGCLVKDYFPEP | SRSGTDFTLTISSLQP | AMKCFLLELQVISHESGDTDAHDTVEDLIILA |
| | VTVSWNSGSLTSGVHTFPAVLQSSGLYSLSS | EDFATYYCLQDYTYPY | NNILSSNGNITESGCKECEELEEKNIKEFLQS |
| | VVTVPSSSLGTQTYVCNVNHKPSNTKVDKRV | TFGQGTKLEIKRAVAA | FVHIVQMFINTSTSGGGSKPPTCPPCPAPEAA |
| | EIKTCGGGSKPPTCPPCPAPEAAGGPSVFLF | PSVFIFPPSEDQVKSG | GGPSVFLFPPKPKDTLMISRTPEVTCVVVDVS |
| | PPKPKDTLMISRTPEVTCVVVDVSQEDPDVK | TVSVVCLLNNFYPREA | QEDPDVKFNWYVNGAEVHHAQTKPRETQYNST |
| | FNWYVNGAEVHHAQTKPRETQYNSTYRVVSV | SVKWKVDGVLKTGNSQ | YRVVSVLTVTHQDWLNGKEYTCKVSNKALPAS |
| | LTVTHQDWLNGKEYTCKVSNKALPASIQKTI | ESVTEQDSKDNTYSLS | IQKTISKDKGQPREPQVYTLPPSREELTKNQV |
| | SKDKGQPREPQVYTLPPSREELTKNQVSLSC | STLTLSSTDYQSHNVY | SLWCLVKGFYPSDIVVEWESSGQPENTYKTTP |
| | AVKGFYPSDIVVEWESSGQPENTYKTTPPVL | ACEVTHQGLSSPVTKS | PVLDSDGSYFLYSKLTVDKSRWQQGNVFSCSV |
| | DSDGSYFLVSKLTVDKSRWQQGNVFSCSVMH | FNRGEC | MHEALHNHYTQKSLSVSPGK |
| | EALHNRYTQKSLSVSPGK | | |

### 5. Conjugated IL-15v and CD4-Targeted IL-15v

Any of the IL-15v and CD4-binding IL-15v molecules described herein may be conjugated antigen binding molecules which are bound to various molecules (e.g., labels) including without limitation macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g. ⁹⁰Y, ¹³¹I, ¹²⁵I, ³⁵S, ³H, ¹²¹In, ⁹⁹Tc), fluorescent substances (e.g., fluorescein and rhodamine), luminescent substances (e.g., luminol), haptens, enzymes (e.g., glucose oxidase), metal chelates, biotin, avidin, and drugs.

The above-described conjugated IL-15v and CD4-binding IL-15v molecules can be prepared according to known methods, e.g., performing chemical modifications on the antigen binding molecules or the lower molecular weight forms thereof described herein. Methods for modifying antibodies are well known in the art (e.g., U.S. Pat. Nos. 5,057,313 and 5,156,840).

### 6. Polynucleotides

Provided are polynucleotides encoding the IL-15v, the IL-15Rα Sushi domain-IL-15v fusion proteins, or the CD4-binding IL-15v molecules, described herein, vectors comprising such polynucleotides, and host cells (e.g., mammalian cells, plant cells, yeast cells, bacteria cells including E. coli cells) comprising such polynucleotides or expression vectors. Provided herein are polynucleotides comprising nucleotide sequence(s) encoding any of the IL-15v, the IL-15Rα Sushi domain-IL-15v fusion proteins, or the CD4-binding IL-15v molecules provided herein, as well as expression cassettes and vector(s) comprising such polynucleotide sequences, e.g., expression vectors for their efficient expression in host cells, e.g., mammalian cells.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by either or both of naturally occurring and non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired host cell.

A “substitution,” as used herein, denotes the replacement of one or more amino acids or nucleotides by different amino acids or nucleotides, respectively.

A polynucleotide “variant,” as the term is used herein, is a polynucleotide that typically differs from a polynucleotide specifically disclosed herein in one or more substitutions, deletions, additions and insertions. Such variants may be naturally occurring or may be synthetically generated, for example, by modifying one or more of the polynucleotide sequences described herein and evaluating one or more biological activities of the encoded polypeptide as described herein, e.g., using any of a number of techniques well known in the art.

An “isolated” nucleic acid molecule has been separated from a component of its natural environment. An isolated nucleic acid molecule can be expressed from cells that ordinarily contain the nucleic acid molecule, but the nucleic acid molecule is present extra-chromosomally or at a chromosomal location that is different from its natural chromosomal location.

Further provided are polynucleotides or nucleic acid molecules encoding an IL-15v or CD4-targeted IL-15v, as described herein. In some embodiments, the polynucleotides encode an immunoglobulin heavy chain variable region (or a fragment thereof) and an immunoglobulin light chain variable region (or a fragment thereof), of the anti-CD4 binding domain and an IL-15v or IL-15Rα Sushi domain-IL-15v fusion protein. In other embodiments, the polynucleotides or nucleic acid molecules are DNA, cDNA, or mRNA. In some other embodiments, the polynucleotides or nucleic acid molecules are codon-biased to enhance expression in a desired host cell.

In some embodiments, provided are polynucleotides encoding the IL-15v, the IL-15Rα Sushi domain-IL-15v fusion proteins or the CD4-targeted IL-15v molecules, described herein. In certain instances, the polynucleotides encode one or more of the IL-15v or IL-15Rα Sushi domain-IL-15v fusion proteins set forth in one or more of Tables C, D and E, as described herein. In certain instances, the polynucleotides encode one or more of CDRs, VH, VL, HC, LC and Fc regions comprising the amino acid sequences of the CD4-targeted IL-15v molecules set forth in one or more of Tables A1-A4, B, F and G, as described herein.

Provided herein are polynucleotides encoding the CDRs, VH, VL, light chain, or heavy chain of a CD4-binding IL-15v molecule, described herein. In one embodiment, the polynucleotides can comprise polynucleotide sequences encoding a heavy chain or heavy chain variable domain binding CD4 comprising the VH CDRs or VH of the CD4 binding domains described herein (see, e.g., Tables A1-A4 and B). In one embodiment, the polynucleotides can comprise polynucleotide sequences encoding a light chain or light chain variable domain binding CD4 comprising the VL CDRs or VL of the CD4 binding domains described herein (see, e.g., Tables A1-A4 and B). In one embodiment, the polynucleotides can comprise polynucleotide sequences encoding a heavy chain and a light chain or a heavy chain variable region and a light chain variable domain binding CD4 comprising the VH/VL CDRs or VH/VL of the CD4 binding domains described herein (see, e.g., Tables A1-A4 and B). In one embodiment, the polynucleotides can comprise polynucleotide sequences encoding the heavy chain comprising the antigen binding domain that specifically binds to CD4, the light chain comprising the antigen binding domain that specifically binds to CD4 and the IL15RASushi-IL15v-Fc fusion protein of a CD4-targeted IL-15v, as described in Table G.

Also encompassed by this disclosure are polynucleotides encoding IL-15v or CD4-binding IL-15v molecules described herein, that have at least one of codon-biased sequences for improved expression in a desired host cell, replacement heterologous signal sequences, and reduced or eliminated mRNA instability elements. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the IL-15v or CD4-binding IL-15v antigen binding molecules in desired host cells is provided, e.g., at kazusa.or.jp/codon/; and genscript.com/tools/codon-frequency-table.

As appropriate, in certain embodiments, the 3′-end of the polynucleotides encoding the IL-15v or CD4-binding IL-15v molecules comprises one or multiple tandem stop codons, e.g., two or more tandem TAG (“amber”), TAA (“ochre”) or TGA (“opal” or “umber”) stop codons. The multiple tandem stop codons can be the same or different.

In some embodiments, the one or more polynucleotides (e.g., mRNA) encoding the IL-15v or the CD4-targeted IL-15v molecules, described herein, are formulated or encapsulated in a lipoplex, e.g., a lipid nanoparticle (LNP). As used herein, a “lipoplex” refers to cationic liposomes that are nonviral (synthetic) lipid carriers of DNA. In some embodiments the lipoplex is a lipid nanoparticle (LNP). As used herein, the term “lipid nanoparticle” refers to one or more spherical nanoparticles with an average diameter of between 10 to 1000 nanometers, and which comprise a solid lipid core matrix that can solubilize lipophilic molecules. In certain embodiments, the lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, each of which is incorporated by reference in its entirety. LNP-encapsulated mRNA molecules encoding IL-15v are described, e.g., in WO 2024/199355, and can be used to encapsulate the herein described IL-15v, IL-15Rα Sushi domain-IL-15v fusion protein or CD4-targeted IL-15v. In certain embodiments, the one or more polynucleotides encoding the antibodies or antigen-binding fragments, described herein, are formulated or encapsulated in an LNP comprised of an ionizable cationic lipid/phosphatidylcholine/cholesterol/PEG-lipid, e.g., in molar ratios of about 50:10:38.5:1.5 mol mol⁻¹, respectively.

In some embodiments, the polynucleotide encoding the heavy chain comprising the antigen binding domain that specifically binds to CD4 comprises a nucleic acid sequence selected from the group consisting of SEQ ID NOs: 1104, 1107, 1109, 1112, 1115, 1118 and 1228, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1104, 1107, 1109, 1112, 1115, 1118 and 1228. In some embodiments, the polynucleotide encoding the light chain comprising the antigen binding domain that specifically binds to CD4 comprises a nucleic acid sequence selected from the group consisting of SEQ ID NOs: 1105, 1110, 1113, 1116 and 1229, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1105, 1110, 1113, 1116 and 1229. In some embodiments, the polynucleotide encoding the IL15RASushi-IL15v-Fc fusion protein comprises a nucleic acid sequence selected from the group consisting of SEQ ID NOs: 1106, 1108, 1111, 1114, 1117, 1119 and 1230, or a nucleic acid sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from the group consisting of SEQ ID NOs: 1106, 1108, 1111, 1114, 1117, 1119 and 1230. In some embodiments, the polynucleotide or polynucleotides encode the heavy chain comprising the antigen binding domain that specifically binds to CD4, the light chain comprising the antigen binding domain that specifically binds to CD4 and the IL15RASushi-IL15v-Fc fusion protein of a CD4-targeted IL-15v, as described herein, and comprise the following polynucleotide sequences, or polynucleotide sequences that are at least 80%, at least 85%, at least 90%, at least 91%, at least 92%, at least 93%, at least 94%, at least 95%, at least 96%, at least 97%, at least 98%, or at least 99% identical to the polynucleotide sequences set forth, respectively: SEQ ID NOs: 1104, 1105 and 1106; SEQ ID NOs: 1107, 1105 and 1108; SEQ ID NOs: 1109, 1110 and 1111; SEQ ID NOs: 1112, 1113 and 1114; SEQ ID NOs: 1115, 1116 and 1117; SEQ ID NOs: 1118, 1113 and 1119; or SEQ ID NOs: 1228, 1229 and 1230.

Illustrative polynucleotide sequences encoding the IL-15v, IL-15Rα Sushi domain-IL-15v fusion protein or CD4-targeted IL-15v, as described herein, are provided in Table I.

TABLE I
polynucleotide sequences of illustrative CD4-targeted IL-15 molecules

| | | | |
|---|---|---|---|
| Ab | | CD4-binding | IL-15Rα Sushi domain-IL-15-Fc |
| Name | CD4-binding domain HC | domain LC | fusion protein |

Binds to CD4 D1

| | | | |
|---|---|---|---|
| 100 | SEQ ID NO: 1104 | SEQ ID NO: 1105 | SEQ ID NO: 1106 |
| | gaagtgcagctgctggaatctggcggaggactg | gacatccagatgaccc | atcacatgccctccacctatgagcgtggaaca |
| | gttcaacctggcggctctctgagactgtcttgt | agagccccagctccct | cgccgacatctgggtcaagagctacagcctgt |
| | gccgccagcggcttcagcttcagcaactacgcc | gagcgccagcgtgggc | acagcagagagcggtacatctgcaacagcggc |
| | atgagctgggtccgacaggcccctggaaaaggc | gacagggtgaccatca | ttcaagagaaaggccggcaccagcagcctgac |
| | cttgaatgggtcgccagcatcaacgaaggcggc | cctgcagagccagcca | cgagtgtgtgctgaacaaggccaccaatgtgg |
| | agcaccttttacgccgacagcgtgaagtcccgg | ggacatcaacaagtac | cccactggaccacacctagcctgaagtgtatt |
| | ttcaccatcagccgggacaacagcaagaacacc | atcgcctggtatcagc | agaggcggcggaggatctggcggaggtggaag |
| | ctgtacctggaaatgaacagcctgagagccgag | agaagcccggcaaggg | cggaggcggaggaagcggtggcggcggtagtg |
| | gacaccgccgtgttctactgcagcagacactac | ccccaaacttttgatc | gcggtggtggatctaattgggtcaacgtgatc |
| | ggcggcagctacgaccccatggattattggggc | cactacactagcaccc | agcgacctgaagaagatcgaggacctgatcca |
| | cagggcaccaccgtgacagtgtctagcgcctct | tgcaccccggagtgcc | gagcatgcacatcgacgccacactgtacaccg |
| | acaaagggccccagcgttttcccactggctcct | cagcaggttcagcggc | agagcgacgtgcaccctagctgtaaagtgacc |
| | agcagcaagagcacaagcggaggaacagccgct | agtggcagcggcaccg | gccatgaagtgctttctgctggaactgcaagt |
| | ctgggctgtctggtcaaggactactttcccgag | actacaccctcaccat | gatcagcctggaaagcggcgacgccagcatcc |
| | cctgtgaccgtgtcctggaatagcggagcactg | tagcagcctgcaaccc | acgataccgtggaagatctgatcatcctggcc |
| | acaagcggcgtgcacacctttccagctgtgctg | gaggacttcgccacct | aacaacagcctgagcagcaacggcaatgtgac |
| | caaagcagcggcctgtactctctgagcagcgtg | actactgcctgcagta | cgagtccggctgcaaagagtgcgaggaactgg |
| | gtcacagtgccaagctctagcctgggcacccag | cgacaaccccctgtac | aagagaagaatatcaaagagttcctgcagagc |
| | acctacatctgcaatgtgaaccacaagcctagc | accttcggccagggca | ttcgtgcacatcgtgcagatgttcatcaacac |
| | aacaccaaggtggacaagaaggtggaacccaag | ccaagttggagatcaa | cagcaccagcggcggcagcgacaagacacata |
| | agctgcgacaagacccacacctgtcctccatgt | gcggaccgtggccgcc | cctgtcctccatgtcctgctccagaagctgct |
| | cctgctccagaagctgcaggcggcccttccgtg | cccagcgtgttcatct | ggcggcccttccgtgtttctgttccctccaaa |
| | tttctgttccctccaaagcctaaggacaccctg | tccctcccagcgacga | gcctaaggacaccctgatgatctccagaactc |
| | atgatcagcagaacccctgaagtgacctgcgtg | gcagctgaagtctggc | ccgaagtgacctgcgtggtggtggacgtgtcc |
| | gtggtggatgtgtcccacgaggatcccgaagtg | accgccagcgtggtgt | cacgaagatcctgaagtgaagttcaattggta |
| | aagttcaattggtacgtggacggcgtggaagtg | gcctgctgaacaactt | cgtggacggcgtggaagtgcacaacgccaaga |
| | cacaacgccaagaccaagcctagagaggaacag | ctacccccgcgaggcc | ccaagcctagagaggaacagtacaacagcacc |
| | tacaacagcacctacagagtggtgtccgtgctg | aaggtgcagtggaagg | tacagagtggtgtccgtgctgaccgtgctgca |
| | accgtgctgcaccaggattggctgaacggcaaa | tggacaacgccctgca | ccaggattggctgaacggcaaagagtacaagt |
| | gagtacaagtgcaaggtgtccaacaaggccctg | gagcggcaacagccag | gcaaggtgtccaacaaggccctgcctgccagc |
| | cctgccagcatcgagaaaaccatcagcaaggcc | gagagcgtgaccgagc | atcgagaaaaccatcagcaaggccaagggcca |
| | aagggccagcctagggaaccccaggtttacaca | aggactccaaggacag | gcctagggaaccccaggtttacacactgcctc |
| | ctgcctccaagccgggaagagatgaccaagaac | cacctacagcctgagc | caagccgggaagagatgaccaagaaccaggtg |
| | caggtgtccctgagctgtgccgtgaagggcttc | agcaccctgaccctga | tccctgtggtgcctggtcaagggcttctaccc |
| | tacccttccgatatcgccgtggaatgggagagc | gcaaggccgactacga | ttccgatatcgccgtggaatgggagagcaatg |
| | aatggccagccagagaacaactacaagacaacc | gaagcacaaggtgtac | gccagcctgagaacaactacaagacaacccct |
| | cctcctgtgctggacagcgacggctcattcttc | gcctgcgaggtgaccc | cctgtgctggacagcgacggctcattcttcct |
| | ctggtgtccaagctgacagtggacaagtccaga | accagggactgtctag | gtactccaagctgacagtggacaagagcagat |
| | tggcagcagggcaacgtgttcagctgcagcgtg | ccccgtgaccaagagc | ggcagcagggcaacgtgttcagctgcagcgtg |
| | atgcacgaggccctgcacaaccggtacacccag | ttcaaccggggcgagt | atgcatgaggccctgcacaaccactacaccca |
| | aagtctctgtctctgagccccggcaaa | gc | gaagtccctgagcctgtctcctggcaaa |
| 100 | SEQ ID NO: 1228 | SEQ ID NO: 1229 | SEQ ID NO: 1230 |
| | gaagtgcagctgctggaatctggcggaggactg | gacatccagatgaccc | atcacatgccctccacctatgagcgtggaaca |
| | gttcaacctggcggctctctgagactgtcttgt | agagccccagctccct | cgccgacatctgggtcaagagctacagcctgt |
| | gccgccagcggcttcagcttcagcaactacgcc | gagcgccagcgtgggc | acagcagagagcggtacatctgcaacagcggc |
| | atgagctgggtccgacaggcccctggaaaaggc | gacagggtgaccatca | ttcaagagaaaggccggcaccagcagcctgac |
| | cttgaatgggtcgccagcatcaacgaaggcggc | cctgcagagccagcca | cgagtgtgtgctgaacaaggccaccaatgtgg |
| | agcaccttttacgccgacagcgtgaagtcccgg | ggacatcaacaagtac | cccactggaccacacctagcctgaagtgtatt |
| | ttcaccatcagccgggacaacagcaagaacacc | atcgcctggtatcagc | agaggcggcggaggatctggcggaggtggaag |
| | ctgtacctggaaatgaacagcctgagagccgag | agaagcccggcaaggg | cggaggcggaggaagcggtggcggcggtagtg |
| | gacaccgccgtgttctactgcagcagacactac | ccccaaacttttgatc | gcggtggtggatctaattgggtcaacgtgatc |
| | ggcggcagctacgaccccatggattattggggc | cactacactagcaccc | agcgacctgaagaagatcgaggacctgatcca |
| | cagggcaccaccgtgacagtgtctagcgcctct | tgcaccccggagtgcc | gagcatgcacatcgacgccacactgtacaccg |
| | acaaagggccccagcgttttcccactggctcct | cagcaggttcagcggc | agagcgacgtgcaccctagctgtaaagtgacc |
| | agcagcaagagcacaagcggaggaacagccgct | agtggcagcggcaccg | gccatgaagtgctttctgctggaactgcaagt |
| | ctgggctgtctggtcaaggactactttcccgag | actacaccctcaccat | gatcagcctggaaagcggcgacgccagcatcc |
| | cctgtgaccgtgtcctggaatagcggagcactg | tagcagcctgcaaccc | acgataccgtggaagatctgatcatcctggcc |
| | acaagcggcgtgcacacctttccagctgtgctg | gaggacttcgccacct | aacaacagcctgagcagcaacggcaatgtgac |
| | caaagcagcggcctgtactctctgagcagcgtg | actactgcctgcagta | cgagtccggctgcaaagagtgcgaggaactgg |
| | gtcacagtgccaagctctagcctgggcacccag | cgacaaccccctgtac | aagagaagaatatcaaagagttcctgcagagc |
| | acctacatctgcaatgtgaaccacaagcctagc | accttcggccagggca | ttcgtgcacatcgtgcagatgttcatcaacac |
| | aacaccaaggtggacaagaaggtggaacccaag | ccaagttggagatcaa | cagcaccagcggcggcagcgacaagacacata |
| | agctgcgacaagacccacacctgtcctccatgt | gcggaccgtggccgcc | cctgtcctccatgtcctgctccagaagctgct |
| | cctgctccagaagctgcaggcggcccttccgtg | cccagcgtgttcatct | ggcggcccttccgtgtttctgttccctccaaa |
| | tttctgttccctccaaagcctaaggacaccctg | tccctcccagcgacga | gcctaaggacaccctgatgatctccagaactc |
| | atgatcagcagaacccctgaagtgacctgcgtg | gcagctgaagtctggc | ccgaagtgacctgcgtggtggtggacgtgtcc |
| | gtggtggatgtgtcccacgaggatcccgaagtg | accgccagcgtggtgt | cacgaagatcctgaagtgaagttcaattggta |
| | aagttcaattggtacgtggacggcgtggaagtg | gcctgctgaacaactt | cgtggacggcgtggaagtgcacaacgccaaga |
| | cacaacgccaagaccaagcctagagaggaacag | ctacccccgcgaggcc | ccaagcctagagaggaacagtacaacagcacc |
| | tacaacagcacctacagagtggtgtccgtgctg | aaggtgcagtggaagg | tacagagtggtgtccgtgctgaccgtgctgca |
| | accgtgctgcaccaggattggctgaacggcaaa | tggacaacgccctgca | ccaggattggctgaacggcaaagagtacaagt |
| | gagtacaagtgcaaggtgtccaacaaggccctg | gagcggcaacagccag | gcaaggtgtccaacaaggccctgcctgccagc |
| | cctgccagcatcgagaaaaccatcagcaaggcc | gagagcgtgaccgagc | atcgagaaaaccatcagcaaggccaagggcca |
| | aagggccagcctagggaaccccaggtttacaca | aggactccaaggacag | gcctagggaaccccaggtttacacactgcctc |
| | ctgcctccaagccgggaagagatgaccaagaac | cacctacagcctgagc | caagccgggaagagatgaccaagaaccaggtg |
| | caggtgtccctgagctgtgccgtgaagggcttc | agcaccctgaccctga | tccctgtggtgcctggtcaagggcttctaccc |
| | tacccttccgatatcgccgtggaatgggagagc | gcaaggccgactacga | ttccgatatcgccgtggaatgggagagcaatg |
| | aatggccagccagagaacaactacaagacaacc | gaagcacaaggtgtac | gccagcctgagaacaactacaagacaacccct |
| | cctcctgtgctggacagcgacggctcattcttc | gcctgcgaggtgaccc | cctgtgctggacagcgacggctcattcttcct |
| | ctggtgtccaagctgacagtggacaagtccaga | accagggactgtctag | gtactccaagctgacagtggacaagagcagat |
| | tggcagcagggcaacgtgttcagctgcagcgtg | ccccgtgaccaagagc | ggcagcagggcaacgtgttcagctgcagcgtg |
| | atgcacgaggccctgcacaaccggtacacccag | ttcaaccggggcgagt | atgcatgaggccctgcacaaccactacaccca |
| | aagtctctgtctctgagccccggcaaa | gc | gaagtccctgagcctgtctcctggcaaa |
| 101 | SEQ ID NO: 1107 | SEQ ID NO: 1105 | SEQ ID NO: 1108 |
| | gaagtgcagctgctggaatctggcggaggactg | gacatccagatgaccc | atcacatgccctccacctatgagcgtggaaca |
| | gttcaacctggcggctctctgagactgtcttgt | agagccccagctccct | cgccgacatctgggtcaagagctacagcctgt |
| | gccgccagcggcttcagcttcagcaactacgcc | gagcgccagcgtgggc | acagcagagagcggtacatctgcaacagcggc |
| | atgagctgggtccgacaggcccctggaaaaggc | gacagggtgaccatca | ttcaagagaaaggccggcaccagcagcctgac |
| | cttgaatgggtcgccagcatcaacgaaggcggc | cctgcagagccagcca | cgagtgtgtgctgaacaaggccaccaatgtgg |
| | agcaccttttacgccgacagcgtgaagtcccgg | ggacatcaacaagtac | cccactggaccacacctagcctgaagtgtatt |
| | ttcaccatcagccgggacaacagcaagaacacc | atcgcctggtatcagc | agaggcggcggaggatctggcggaggtggaag |
| | ctgtacctggaaatgaacagcctgagagccgag | agaagcccggcaaggg | cggaggcggaggaagcggtggcggcggtagtg |
| | gacaccgccgtgttctactgcagcagacactac | ccccaaacttttgatc | gcggtggtggatctaattgggtcaacgtgatc |
| | ggcggcagctacgaccccatggattattggggc | cactacactagcaccc | agcgacctgaagaagatcgaggacctgatcca |
| | cagggcaccaccgtgacagtgtctagcgcctct | tgcaccccggagtgcc | gagcatgcacatcgacgccacactgtacaccg |
| | acaaagggccccagcgttttcccactggctccc | cagcaggttcagcggc | agagcgacgtgcaccctagctgtaaagtgacc |
| | tgtagcagaagcaccagcgaatctacagccgct | agtggcagcggcaccg | gccatgaagtgctttctgctggaactgcaagt |
| | ctgggctgcctggtcaaggactactttcctgag | actacaccctcaccat | gatcagcctggaaagcggcgacgccagcatcc |
| | cctgtgaccgtgtcctggaacagcggagcactg | tagcagcctgcaaccc | acgataccgtggaagatctgatcatcctggcc |
| | acatctggcgtgcacacctttccagccgtgctg | gaggacttcgccacct | aacaacagcctgagcagcaacggcaatgtgac |
| | caaagcagcggcctgtactctctgagcagcgtg | actactgcctgcagta | cgagtccggctgcaaagagtgcgaggaactgg |
| | gtcacagtgcctagctctagcctgggcaccaag | cgacaaccccctgtac | aagagaagaatatcaaagagttcctgcagagc |
| | acctacacctgtaatgtggaccacaagcctagc | accttcggccagggca | ttcgtgcacatcgtgcagatgttcatcaacac |
| | aacaccaaggtggacaagcgcgtggaatctaag | ccaagttggagatcaa | cagcggcggcggaggtggaaagtacggccctc |
| | tacggccctccttgtcctccatgtcctgctcca | gcggaccgtggccgcc | cttgtcctccatgtcctgctccagaagctgct |
| | gaagctgcaggcggcccttccgtgtttctgttc | cccagcgtgttcatct | ggcggcccttccgtgtttctgttccctccaaa |
| | cctccaaagcctaaggacaccctgatgatcagc | tccctcccagcgacga | gcctaaggacaccctgatgatctctcggaccc |
| | agaacccctgaagtgacctgcgtggtggtggac | gcagctgaagtctggc | ctgaagtgacctgcgtggtggtggatgtgtcc |
| | gtgtcccaagaggatcctgaggtgcagttcaat | accgccagcgtggtgt | caagaggatcccgaggtgcagttcaattggta |
| | tggtacgtggacggcgtggaagtgcacaacgcc | gcctgctgaacaactt | cgtggacggcgtggaagtgcacaacgccaaga |
| | aagaccaagcctagagaggaacagttcaacagc | ctacccccgcgaggcc | ccaagcctagagaggaacagttcaactccacc |
| | acctacagagtggtgtccgtgctgacagtgctg | aaggtgcagtggaagg | tacagagtggtgtccgtgctgaccgtgctgca |
| | caccaggattggctgaacggcaaagagtacaag | tggacaacgccctgca | ccaggattggctgaacggcaaagagtacaagt |
| | tgcaaggtgtccaacaagggcctgccaagcagc | gagcggcaacagccag | gcaaggtgtccaacaagggcctgccttccagc |
| | atcgagaaaaccatcagcaaggccaagggccag | gagagcgtgaccgagc | atcgaaaagaccatctccaaggccaagggcca |
| | cctagggaaccccaggtttacacactgcctcca | aggactccaaggacag | gcctagggaaccccaggtttacaccctgcctc |
| | agccaagaggaaatgaccaagaaccaggtgtcc | cacctacagcctgagc | caagccaagaggaaatgaccaagaaccaggtg |
| | ctgagctgcgccgtgaagggcttttacccttcc | agcaccctgaccctga | tccctgtggtgcctggtcaagggcttctaccc |
| | gatatcgccgtggaatgggagagcaatggccag | gcaaggccgactacga | ttccgatatcgccgtggaatgggagagcaatg |
| | ccagagaacaactacaagacaacccctcctgtg | gaagcacaaggtgtac | gccagcctgagaacaactacaagaccacacct |
| | ctggacagcgacggctcattcttcctggtgtcc | gcctgcgaggtgaccc | cctgtgctggactccgacggctccttctttct |
| | agactgaccgtggacaagagcagatggcaagag | accagggactgtctag | gtactcccgcctgaccgtggacaagtccagat |
| | ggcaacgtgttcagctgcagcgtgatgcacgag | ccccgtgaccaagagc | ggcaagagggcaacgtgttctcctgctccgtg |
| | gccctgcacaaccggtacacccagaagtctctg | ttcaaccggggcgagt | atgcacgaggccctgcacaatcactacaccca |
| | tccctgtctctgggcaaa | gc | gaagtccctgtctctgtctcttggcaag |
| 101 | SEQ ID NO: 1109 | SEQ ID NO: 1110 | SEQ ID NO: 1111 |
| | gaggtgcagctgctggaatctggcggaggattg | gacatccagatgaccc | atcacctgtcctccacctatgtccgtggaaca |
| | gttcagcctggcggctctctgagactgtcttgt | agtctccatcctctct | cgccgacatctgggtcaagtcctacagcctgt |
| | gccgcctctggcttctccttctccaactacgcc | gtccgcctctgtgggc | actccagagagcggtacatctgcaactccggc |
| | atgtcctgggtccgacaggctcctggcaaagga | gacagagtgaccatca | ttcaagagaaaggccggcacctctagcctgac |
| | ctggaatgggtcgcctccatcaacgaaggcggc | cctgtagagccagcca | cgagtgcgtgctgaacaaggccaccaatgtgg |
| | tctaccttctacgccgactccgtgaagtcccgg | ggacatcaacaagtat | cccactggaccacacctagcctgaagtgtatt |
| | ttcaccatctccagagacaactccaagaacacc | atcgcctggtatcagc | agaggcggcggaggatctggcggaggtggaag |
| | ctgtacctggaaatgaactccctgagagccgag | agaagcccggcaaggg | tggcggtggtggttcaggcggtggcggaagcg |
| | gacaccgccgtgttctactgctctagacactac | acctaagctgctgatc | gaggcggtggatctaattgggtcaacgtgatc |
| | ggcggcagctacgaccccatggattattggggc | cactacacctccacac | tccgacctgaagaagatcgaggacctgatcca |
| | cagggcaccacagtgaccgtgtcctctgcttct | tgcaccctggcgtgcc | gtccatgcacatcgacgctaccctgtacaccg |
| | accaagggacccagcgtgttccctctggctcct | ctctagattttccggc | agtccgacgtgcacccttcctgtaaagtgacc |
| | tgctccagatccacctccgagtctacagctgct | tctggctctggcaccg | gccatgaagtgctttctgctggaactgcaagt |
| | ctgggctgcctggtcaaggactactttcctgag | actataccctgacaat | gatctccctggaatccggcgacgcctctatcc |
| | cctgtgacagtgtcctggaactccggcgctctg | ctccagcctgcagcct | acgacaccgtggaagatctgatcatcctggcc |
| | acatctggcgtgcacacctttccagctgtgctg | gaggacttcgccacct | aacaactccctgtccagcaacggcaacgtgac |
| | cagtcctccggcctgtactctctgtcctctgtc | actactgcctgcagta | cgagtctggctgcaaagagtgcgaggaactgg |
| | gtgaccgtgccttcctctagcctgggcaccaag | cgacaaccctctgtac | aagagaagaacatcaaagagttcctccagtcc |
| | acctacacctgtaatgtggaccacaagccttcc | acctttggccagggca | ttcgtgcacatcgtgcagatgttcatcaacac |
| | aacaccaaggtggacaagcgcgtggaatctaag | ccaagctggaaatcaa | ctccggcggaggcggagggaagtatggacctc |
| | tacggccctccttgtcctccatgtccagctcca | gcggacagtggccgct | cttgtcctccatgtcctgctccagaagctgct |
| | gaagctgctggcggcccttccgtgtttctgttc | ccttccgtgttcatct | ggcggcccttccgtgtttctgttccctccaaa |
| | cctccaaagcctaaggacaccctgatgatctct | tcccaccttccgacga | gcctaaggacaccctgatgatctctcggaccc |
| | cggacccctgaagtgacctgcgtggtggtggat | gcagctgaagtccggc | ctgaagtgacctgcgtggtggtggatgtgtcc |
| | gtgtcccaagaggatcccgaggtgcagttcaat | acagcttctgtcgtgt | caagaggatcccgaggtgcagttcaattggta |
| | tggtacgtggacggcgtggaagtgcacaacgcc | gcctgctgaacaactt | cgtggacggcgtggaagtgcacaacgccaaga |
| | aagaccaagcctagagaggaacagttcaactcc | ctaccctcgggaagct | ccaagcctagagaggaacagttcaactccacc |
| | acctacagagtggtgtccgtgctgaccgtgctg | aaggtgcagtggaagg | tacagagtggtgtccgtgctgaccgtgctgca |
| | caccaggattggctgaacggcaaagagtacaag | tggacaacgccctgca | ccaggattggctgaacggcaaagagtacaagt |
| | tgcaaggtgtccaacaagggcctgccttccagc | gtccggcaactcccaa | gcaaggtgtccaacaagggcctgccttccagc |
| | atcgaaaagaccatcagcaaggccaagggccag | gagtctgtgaccgagc | atcgaaaagaccatctccaaggccaagggcca |
| | cctagggaaccccaggtttacaccctgcctcca | aggactccaaggacag | gcctagggaaccccaggtttacaccctgcctc |
| | agccaagaggaaatgaccaagaaccaggtgtcc | cacctacagcctgtcc | caagccaagaggaaatgaccaagaaccaggtg |
| | ctgtcctgcgccgtgaagggcttctacccttct | agcactctgaccctgt | tccctgtggtgcctggtcaagggcttctaccc |
| | gatatcgccgtggaatgggagagcaacggccag | ccaaggccgactacga | ttccgatatcgccgtggaatgggagagcaatg |
| | cctgagaacaactacaagacaacccctcctgtg | gaagcacaaggtgtac | gccagcctgagaacaactacaagacaacccct |
| | ctggactccgacggctctttctttctggtgtcc | gcctgcgaagtgaccc | cctgtgctggactccgacggctccttctttct |
| | cgcctgaccgtggacaagtctagatggcaagag | atcagggcctgtctag | gtactcccgcctgaccgtggacaagtccagat |
| | ggcaacgtgttctcctgcagcgtgatgcacgag | ccctgtgaccaagtct | ggcaagagggcaacgtgttctcctgctccgtg |
| | gccctgcacaacagatacacccagaagtccctg | ttcaaccggggcgagt | atgcacgaggccctgcacaatcactacaccca |
| | tctctgtccctgggcaaa | gt | gaagtccctgtctctgtccctgggcaaa |

Binds to CD4 D3

| | | | |
|---|---|---|---|
| 173 | SEQ ID NO: 1112 | SEQ ID NO: 1113 | SEQ ID NO: 1114 |
| | gaggtcgagctcctggagagcggcgggggcctc | gcgatccagatgaccc | atcacatgccctccacctatgagcgtggaaca |
| | gtccagcccggcggcagcctcagactgagctgc | aatccccctcctctct | cgccgacatctgggtcaagagctacagcctgt |
| | gcggctagtggactgaccttttccacctttgcc | gagcgcaagtgttggc | acagcagagagcggtacatctgcaacagcggc |
| | atgtcatgggtgcggcaggcgccaggcaaagga | gaccgagtgacgatca | ttcaagagaaaggccggcaccagcagcctgac |
| | ctggagtgggtatctggaattagcggctccggg | cttgtagggcctcaca | cgagtgtgtgctgaacaaggccaccaatgtgg |
| | gagaacacatattacgctgactctgtaaaagga | tggcatacggaacgac | cccactggaccacacctagcctgaagtgtatt |
| | aggtttacgatcactagtgataactccaagaat | ctgggctggtatcagc | agaggcggcggaggatctggcggaggtggaag |
| | actctttatctgcagatgaatagcctgagggca | agaagccaggaaaggc | cggaggcggaggaagcggtggcggcggtagtg |
| | gaggatacagccgtgtactattgcgccaaggaa | acccaaagtgctgatt | gcggtggtggatctaattgggtcaacgtgatc |
| | ggctacaattggaactacatggacgtgtggggg | tatgaagcttcatcac | ggcgacctgaagaagatcgaggacctgatcca |
| | aagggcaccacagtcaccgtatcttccgccagc | ttcagtctggggtgcc | gagcatgcacatcgacgccacactgtacaccg |
| | accaagggccctagtgtgttccctctggctccc | cagtcgattttcaggt | agagcgacgtgcaccctagctgtaaagtgacc |
| | agcagcaagtctacatctggcggaacagccgct | tcaagatccgggaccg | gccatgaagtgctttctgctggaactgcaagt |
| | ctgggctgcctggtcaaggattactttcccgag | acttcactctcaccat | gatcagcctggaaagcggcgacgccagcatcc |
| | cctgtgaccgtgtcctggaattctggcgctctg | cagctccttgcagccc | acgataccgtggaagatctgatcatcctggcc |
| | acaagcggcgtgcacacctttccagctgtgctg | gaggacttcgcgacct | aacaacagcctgagcagcaacggcaatgtgac |
| | caaagcagcggcctgtactctctgagcagcgtg | actattgtcttcagga | cgagtccggctgcaaagagtgcgaggaactgg |
| | gtcacagtgcctagctctagcctgggcacccag | ctacacatatccatat | aagagaagaatatcaaagagttcctgcagagc |
| | acctacatctgcaatgtgaaccacaagcctagc | actttcggtcagggga | ttcgtgcacatcgtgcagatgttcatcaacac |
| | aacaccaaggtggacaagaaggtggaacccaag | cgaaacttgagataaa | ctctacctctggtggttctgacaagacccaca |
| | agctgcgacaagacccacacctgtcctccatgt | aagaacagtggccgct | cctgtcctccatgtcctgctccagaagctgct |
| | cctgctccagaagctgctggcggcccttccgtg | ccatctgtcttcatct | ggcggcccttccgtgtttctgttccctccaaa |
| | tttctgttccctccaaagcctaaggacaccctg | tcccgccatctgatga | gcctaaggacaccctgatgatcagcagaaccc |
| | atgatcagcagaacccctgaagtgacctgcgtg | gcagttgaaatctgga | ctgaagtgacctgcgtggtggtggatgtgtcc |
| | gtggtggatgtgtcccacgaggatcccgaagtg | actgcctctgttgtgt | cacgaggatcccgaagtgaagttcaattggta |
| | aagttcaattggtacgtggacggcgtggaagtg | gcctgctgaataactt | cgtggacggcgtggaagtgcacaacgccaaga |
| | cacaacgccaagaccaagcctagagaggaacag | ctatcccagagaggcc | ccaagcctagagaggaacagtacaacagcacc |
| | tacaacagcacctacagagtggtgtccgtgctg | aaagtacagtggaagg | tacagagtggtgtccgtgctgaccgtgctgca |
| | accgtgctgcaccaggattggctgaacggcaaa | tggataacgccctcca | ccaggattggctgaacggcaaagagtacaagt |
| | gagtacaagtgcaaggtgtccaacaaggccctg | atcgggtaactcccag | gcaaggtgtccaacaaggccctgcctgccagc |
| | cctgccagcatcgagaaaaccatcagcaaggcc | gagagtgtcacagagc | atcgagaaaaccatcagcaaggccaagggcca |
| | aagggccagcctagggaaccccaggtttacaca | aggacagcaaggacag | gcctagggaaccccaggtttacacactgcctc |
| | ctgcctccaagccgggaagagatgaccaagaac | cacctacagcctcagc | caagccgggaagagatgaccaagaaccaggtg |
| | caggtgtccctgagctgtgccgtgaagggcttc | agcaccctgacgctga | tccctgtggtgcctggtcaagggcttctaccc |
| | tacccttccgatatcgccgtggaatgggagagc | gcaaagcagactacga | ttccgatatcgccgtggaatgggagagcaatg |
| | aatggccagcctgagaacaactacaagacaacc | gaaacacaaagtctac | gccagcctgagaacaactacaagacaacccct |
| | cctcctgtgctggacagcgacggctcattcttc | gcctgcgaagtcaccc | cctgtgctggacagcgacggctcattcttcct |
| | ctggtgtccaagctgacagtggacaagtccaga | atcagggcctgagctc | gtacagcaagctgacagtggacaagagcagat |
| | tggcagcagggcaacgtgttcagctgcagcgtg | gcccgtcacaaagagc | ggcagcagggcaacgtgttcagctgcagcgtg |
| | atgcacgaggccctgcacaaccggtacacccag | ttcaacaggggagagt | atgcacgaggccctgcacaaccactacaccca |
| | aagtccctgtctctgagccccggcaaa | gt | gaagtccctgagcctgtctcctggcaaa |
| 173 | SEQ ID NO: 1115 | SEQ ID NO: 1116 | SEQ ID NO: 1117 |
| | gaggtggaactgctggaatctggcggaggattg | gccatccagatgaccc | atcacctgtcctccacctatgtccgtggaaca |
| | gttcagcctggcggctctctgagactgtcttgt | agtctccatcctctct | cgccgacatctgggtcaagtcctacagcctgt |
| | gctgcttctggcctgaccttctccaccttcgcc | gtccgcctctgtgggc | actccagagagcggtacatctgcaactccggc |
| | atgtcctgggtccgacaggctcctggcaaagga | gacagagtgaccatca | ttcaagagaaaggccggcacctctagcctgac |
| | ctggaatgggtgtccggcatctctggctctggc | cctgtagagccagcca | cgagtgcgtgctgaacaaggccaccaatgtgg |
| | gagaatacctactacgccgactccgtgaagggc | cggcatcagaaacgac | cccactggaccacacctagcctgaagtgtatt |
| | agattcaccatcacctccgacaactccaagaac | ctcggctggtatcagc | agaggcggcggaggatctggcggaggtggaag |
| | accctgtacctgcagatgaactccctgagagcc | agaagcctggcaaggc | cggaggcggaggaagcggtggcggcggtagtg |
| | gaggacaccgccgtgtactactgtgccaaagag | tcccaaggtgctgatc | gcggtggtggatctaattgggtcaacgtgatc |
| | ggctacaactggaactacatggacgtgtggggc | tacgaggcttccagtc | ggcgacctgaagaagatcgaggacctgatcca |
| | aagggcaccaccgtgacagtttcttccgcctcc | tgcagtctggcgtgcc | gtccatgcacatcgacgctaccctgtacaccg |
| | accaagggacccagcgttttccctctggctcca | ctctaggttctccggc | agtccgacgtgcacccttcctgtaaagtgacc |
| | tcctccaagtctacctctggcggaacagctgct | tctaggtctggcaccg | gccatgaagtgctttctgctggaactgcaagt |
| | ctgggctgcctggtcaaggactactttcctgag | actttaccctgacaat | gatctccctggaatccggcgacgcctctatcc |
| | cctgtgaccgtgtcctggaactccggtgctctg | ctccagcctgcagcct | acgacaccgtggaagatctgatcatcctggcc |
| | acatctggcgtgcacacctttccagctgtgctg | gaggacttcgccacct | aacaactccctgtccagcaacggcaacgtgac |
| | cagtcctccggcctgtactctctgtcctctgtc | actactgcctgcaaga | cgagtctggctgcaaagagtgcgaggaactgg |
| | gtgaccgtgccttccagctctctgggaacccag | ctacacctatccttac | aagagaagaacatcaaagagttcctccagtcc |
| | acctacatctgcaatgtgaaccacaagccttcc | accttcggccagggca | ttcgtgcacatcgtgcagatgttcatcaacac |
| | aacaccaaggtggacaagaaggtggaacccaag | ccaagctggaaatcaa | ctccaccagcggcggctccgacaagacacata |
| | tcctgcgacaagacccacacctgtcctccatgt | gagaaccgtggccgct | cctgtccaccttgtcctgctccagaggctgct |
| | ccagctccagaagctgctggcggcccttccgtg | ccttccgtgttcatct | ggtggcccttccgtgtttctgttccctccaaa |
| | tttctgttccctccaaagcctaaggacaccctg | tcccaccttccgacga | gcctaaggacaccctgatgatctctcggaccc |
| | atgatctctcggacccctgaagtgacctgcgtg | gcagctgaagtccggc | ctgaagtgacctgcgtggtggtcgatgtgtct |
| | gtggtggatgtgtctcacgaggatcccgaagtg | acagcttctgtcgtgt | cacgaggatcccgaagtgaagttcaattggta |
| | aagttcaattggtacgtggacggcgtggaagtg | gcctgctgaacaactt | cgtggacggcgtggaagtgcacaacgccaaga |
| | cacaacgccaagaccaagcctagagaggaacag | ctaccctcgggaagcc | ccaagcctagagaggaacagtacaactccacc |
| | tacaactccacctacagagtggtgtccgtgctg | aaggtgcagtggaagg | tacagagtggtgtccgtgctgaccgtgctgca |
| | accgtgctgcaccaggattggctgaacggcaaa | tggacaatgctctgca | ccaggattggctgaacggcaaagagtacaagt |
| | gagtacaagtgcaaggtgtccaacaaggccctg | gtccggcaactcccaa | gcaaggtgtccaacaaggccctgcctgcctcc |
| | cctgcctccatcgaaaagaccatctccaaggcc | gagtccgtgaccgagc | atcgaaaagaccatctccaaggccaagggcca |
| | aagggccagcctagggaaccccaggtttacacc | aggactccaaggactc | gcctagggaaccccaggtttacaccctgccac |
| | ctgccacctagccgggaagagatgaccaagaac | tacctacagcctgtcc | ctagccgggaagagatgaccaagaaccaggtg |
| | caggtgtccctgtcctgcgctgtgaagggcttc | tccacactgaccctgt | tccctgtggtgcctggtcaagggcttctaccc |
| | tacccttccgatatcgccgtggaatgggagagc | ccaaggccgactacga | ttccgatatcgccgtggaatgggagagcaatg |
| | aatggccagcctgagaacaactacaagacaacc | gaagcacaaggtgtac | gccagcctgagaacaactacaagacaacccct |
| | cctcctgtgctggactccgacggctcattcttc | gcctgcgaagtgaccc | cctgtgctggactccgacggctcattcttcct |
| | ctggtgtccaagctgacagtggacaagtccaga | atcagggcctgtctag | gtacagcaagctgacagtggacaagtccagat |
| | tggcagcagggcaacgtgttctcctgctccgtg | ccctgtgaccaagtct | ggcagcagggcaacgtgttctcctgctccgtg |
| | atgcacgaggccctgcacaacagatacacccag | ttcaaccggggcgagt | atgcacgaggccctgcacaatcactacaccca |
| | aagtccctgtctctgtcccctggcaaa | gt | gaagtccctgtctctgtcccctggcaaa |
| 175 | SEQ ID NO: 1118 | SEQ ID NO: 1113 | SEQ ID NO: 1119 |
| | gaggtcgagctcctggagagcggcgggggcctc | gcgatccagatgaccc | atcacatgccctccacctatgagcgtggaaca |
| | gtccagcccggcggcagcctcagactgagctgc | aatccccctcctctct | cgccgacatctgggtcaagagctacagcctgt |
| | gcggctagtggactgaccttttccacctttgcc | gagcgcaagtgttggc | acagcagagagcggtacatctgcaacagcggc |
| | atgtcatgggtgcggcaggcgccaggcaaagga | gaccgagtgacgatca | ttcaagagaaaggccggcaccagcagcctgac |
| | ctggagtgggtatctggaattagcggctccggg | cttgtagggcctcaca | cgagtgtgtgctgaacaaggccaccaatgtgg |
| | gagaacacatattacgctgactctgtaaaagga | tggcatacggaacgac | cccactggaccacacctagcctgaagtgtatt |
| | aggtttacgatcactagtgataactccaagaat | ctgggctggtatcagc | agaggcggcggaggatctggcggaggtggaag |
| | actctttatctgcagatgaatagcctgagggca | agaagccaggaaaggc | cggaggcggaggaagcggtggcggcggtagtg |
| | gaggatacagccgtgtactattgcgccaaggaa | acccaaagtgctgatt | gcggtggtggatctaattgggtcaacgtgatc |
| | ggctacaattggaactacatggacgtgtggggg | tatgaagcttcatcac | agcgacctgaagaagatcgaggacctgatcca |
| | aagggcaccacagtcaccgtatcttccgcctct | ttcagtctggggtgcc | gagcatgcacatcgacgccacactgtacaccg |
| | acaaagggccccagcgttttcccactggctccc | cagtcgattttcaggt | agagcgacgtgcaccctagctgtaaagtgacc |
| | tgtagcagaagcaccagcgaatctacagccgct | tcaagatccgggaccg | gccatgaagtgctttctgctggaactgcaagt |
| | ctgggctgcctggtcaaggactactttcctgag | acttcactctcaccat | gatcagcctggaaagcggcgacgccagcatcc |
| | cctgtgaccgtgtcctggaacagcggagcactg | cagctccttgcagccc | acgataccgtggaagatctgatcgccctggcc |
| | acatctggcgtgcacacctttccagccgtgctg | gaggacttcgcgacct | aacaacagcctgagcagcaacggcaatgtgac |
| | caaagcagcggcctgtactctctgagcagcgtg | actattgtcttcagga | cgagtccggctgcaaagagtgcgaggaactgg |
| | gtcacagtgcctagctctagcctgggcaccaag | ctacacatatccatat | aagagaagaatatcaaagagttcctgcagagc |
| | acctacacctgtaatgtggaccacaagcctagc | actttcggtcagggga | ttcgtgcacatcgtgcagatgttcatcaacac |
| | aacaccaaggtggacaagcgcgtggaatctaag | cgaaacttgagataaa | ctctggtggtggcggagggaagtacggacctc |
| | tacggccctccttgtcctccatgtcctgctcca | aagaacagtggccgct | cttgtcctccatgtcctgctccagaagctgct |
| | gaagctgcaggcggcccttccgtgtttctgttc | ccatctgtcttcatct | ggcggcccttccgtgtttctgttccctccaaa |
| | cctccaaagcctaaggacaccctgatgatcagc | tcccgccatctgatga | gcctaaggacaccctgatgatcagcagaaccc |
| | agaacccctgaagtgacctgcgtggtggtggac | gcagttgaaatctgga | ctgaagtgacctgcgtggtggtggacgtgtcc |
| | gtgtcccaagaggatcctgaggtgcagttcaat | actgcctctgttgtgt | caagaggatcctgaggtgcagttcaattggta |
| | tggtacgtggacggcgtggaagtgcacaacgcc | gcctgctgaataactt | cgtggacggcgtggaagtgcacaacgccaaga |
| | aagaccaagcctagagaggaacagttcaacagc | ctatcccagagaggcc | ccaagcctagagaggaacagttcaacagcacc |
| | acctacagagtggtgtccgtgctgacagtgctg | aaagtacagtggaagg | tacagagtggtgtccgtgctgaccgtgctgca |
| | caccaggattggctgaacggcaaagagtacaag | tggataacgccctcca | ccaggattggctgaacggcaaagagtacaagt |
| | tgcaaggtgtccaacaagggcctgccaagcagc | atcgggtaactcccag | gcaaggtgtccaacaagggcctgcctagctcc |
| | atcgagaaaaccatcagcaaggccaagggccag | gagagtgtcacagagc | atcgagaaaaccatcagcaaggccaagggcca |
| | cctagggaaccccaggtttacacactgcctcca | aggacagcaaggacag | gccaagagaaccccaggtgtacacactgcctc |
| | agccaagaggaaatgaccaagaaccaggtgtcc | cacctacagcctcagc | caagccaagaggaaatgaccaagaaccaggtg |
| | ctgagctgcgccgtgaagggcttttacccttcc | agcaccctgacgctga | tccctgtggtgcctggtcaagggcttctaccc |
| | gatatcgccgtggaatgggagagcaatggccag | gcaaagcagactacga | ttccgatatcgccgtggaatgggagagcaatg |
| | ccagagaacaactacaagacaacccctcctgtg | gaaacacaaagtctac | gccagcctgagaacaactacaagacaacccct |
| | ctggacagcgacggctcattcttcctggtgtcc | gcctgcgaagtcaccc | cctgtgctggacagcgacggctcattcttcct |
| | agactgaccgtggacaagagcagatggcaagag | atcagggcctgagctc | gtactccagactgaccgtggacaagagcagat |
| | ggcaacgtgttcagctgcagcgtgatgcacgag | gcccgtcacaaagagc | ggcaagagggcaacgtgttcagctgcagcgtg |
| | gccctgcacaaccggtacacccagaagtctctg | ttcaacaggggagagt | atgcatgaggccctgcacaaccactacaccca |
| | tccctgtctctgggcaaa | gt | gaagtccctgagcctgtctctgggcaaa |

### 7. Vectors

Further provided are vectors comprising one or more polynucleotides encoding an IL-15v, IL-15Rα Sushi domain-IL-15v fusion protein or CD4-targeted IL-15v, as described herein. Also provided are vectors comprising one or more expression cassettes (e.g., multi-cistronic, bicistronic, tricistronic) for expressing one or more of the IL-15v, IL-15Rα Sushi domain-IL-15v fusion protein or CD4-targeted IL-15v, described herein. Moreover, expression cassettes for expressing polypeptide chains of a biologic molecule comprised of two or more polypeptide chains, e.g., two, three or four polypeptide chains, can be expressed from one or multiple expression vectors, e.g., one, two, three or four vectors. A vector can be of any type, for example, a recombinant vector such as an expression vector. Vectors include, but are not limited to, plasmids, cosmids, bacterial artificial chromosomes (BAC) and yeast artificial chromosomes (YAC) and vectors derived from bacteriophages or plant or animal (including human) viruses. Vectors can comprise an origin of replication and one or more selection markers recognized by and operational in the proposed host cell (e.g., including prokaryotic and eukaryotic host cells), and in the case of expression vectors, promoter, terminator, and other regulatory regions recognized by the host cell. In additional embodiments, a vector comprises an expression cassette as described herein, e.g., comprising a polynucleotide encoding an IL-15v, an IL-15Rα Sushi domain-IL-15v fusion protein or a CD4-targeted IL-15v molecule of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include, but are not limited to, those suitable for recombinant production of the antibodies disclosed herein. The term “vector,” as used herein, refers to a nucleic acid molecule capable of propagating another nucleic acid. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The choice of the vector is dependent on the recombinant procedures followed and the host used. Introduction of vectors into host cells can be effected by inter alia calcium phosphate transfection, virus infection, DEAE-dextran-mediated transfection, lipofectamine transfection or electroporation. Vectors may be autonomously replicating or may replicate together with the chromosome into which they have been integrated. In certain embodiments, the vectors contain one or more selection markers. The choice of the markers may depend on the host cells of choice. These include, but are not limited to, kanamycin, neomycin, puromycin, hygromycin, zeocin, ampicillin (AmpR), thymidine kinase gene from Herpes simplex virus (HSV-TK), glutamine synthetase (GS) and dihydrofolate reductase (DHFR) gene. Vectors comprising one or more polynucleotides encoding the antibodies described herein, operably linked to one or more polynucleotides encoding proteins or peptides that can be used to isolate the polypeptides of interest, are also covered by the disclosure. These proteins or peptides include, but are not limited to, glutathione-S-transferase, maltose binding protein, metal-binding polyhistidine, green fluorescent protein, luciferase and beta-galactosidase. In other embodiments, the vector that is used is or is based on pcDNA™3.1+ (ThermoFisher, MA), pCI (Promega, WI), pIRES (Takara, CA), or pCGS3 (Sigma-Aldrich, MO).

### 8. Host Cells

The disclosure also provides host cells comprising one or more recombinant polynucleotides, one or more expression cassettes, or one or more vectors, as described herein. Any of a variety of host cells can be used, including prokaryotic cells (e.g., bacterial cells, e.g., E. coli cells) and eukaryotic cells. In one embodiment, a host cell is a eukaryotic cell, for example, a yeast cell, a plant cell, an insect cell, a mammalian cell. In some embodiments, the host cell is a mammalian cell such as a Chinese Hamster Ovary (CHO)-based or CHO-originated cell (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines), a COS cell, a BHK cell, a NS0 cell or a human cell. Examples of human host cells of use include without limitation, inter alia, HeLa, 911, AT1080, A549, HEK293, Expi293™ and HEK293T-cells.

The terms “host cell,” “host cell line,” and “host cell culture” are used interchangeably and refer to cells into which exogenous nucleic acid could be or has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding an IL-15v, an IL-15Rα Sushi domain-IL-15v fusion protein or a CD4-targeted IL-15v molecule, as described herein. In some embodiments, the host cells described herein express or produce at least 4 g/L, e.g., at least 5 g/L, at least 6 g/L, at least 7 g/L, at least 8 g/L, at least 9 g/L, or more, CD4-targeted IL-15v molecule.

In some embodiments, the host cell predominantly sialylates N-linked glycosylation sites within the variable regions of an immunoglobulin antigen binding domain. In some embodiments, the polynucleotides encoding an IL-15v, an IL-15Rα Sushi domain-IL-15v fusion protein or a CD4-targeted IL-15v molecule, as described herein, are expressed in a host cell that sialylates at least 50%, at least 55%, at least 60%, at least 65%, at least 70%, at least 75%, at least 80%, at least 85%, at least 90%, at least 91%, at least 92%, at least 93%, at least 94%, at least 95%, or more, N-linked glycosylation sites of the expressed molecules. In various embodiments, the IL-15v, the IL-15Rα Sushi domain-IL-15v fusion protein or the CD4-targeted IL-15v molecule expressed from such host cells, at least 50%, at least 60%, at least 70%, least 80%, at least 85%, at least 90%, or more, N-linked glycosylation sites are sialylated. In various embodiments, the N-linked glycosylation sites have a sialic acid occupancy (e.g., a glycan comprising one or two terminal sialic acid residues) of at least 40%, at least 50%, at least 60%, at least 70%, least 80%, at least 85%, at least 90%, or more. In some embodiments, the sialylated N-linked glycosylation sites comprise from 1 to 5 sialic acid residues, e.g., from 1 to 4 sialic acid residues, e.g., from 1 to 3 sialic acid residues, e.g., from 1 to 2 sialic acid residues. In some embodiments, the N-linked glycosylation sites are sialylated with N-acetylneuraminic acid (NANA). In some embodiments, the sialic acid residues are present in biantennary structures. In some embodiments, the sialic acid residues are present in complex N-linked glycan structures. In some embodiments, the sialic acid residues are present in hybrid N-linked glycan structures. In some embodiments, the glycans are terminally sialylated.

If the IL-15v, the IL-15Rα Sushi domain-IL-15v fusion protein or the CD4-targeted IL-15v molecule are to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion of the IL-15v, the IL-15Rα Sushi domain-IL-15v fusion protein or the CD4-targeted IL-15v molecules. For production into the periplasm of E. coli, the pelB signal sequence (Lei et al., J. Bacteriol., 169: 4379 (1987)) may be used as the signal sequence for secretion of the IL-15v, the IL-15Rα Sushi domain-IL-15v fusion protein or the CD4-targeted IL-15v molecules. For bacterial expression, calcium chloride methods or electroporation methods may be used to introduce the expression vector into the bacterial cell.

### 9. Methods of Production

The herein described IL-15v, IL-15Rα Sushi domain-IL-15v fusion proteins and the CD4-targeted IL-15v molecules can be produced by any method known in the art for the synthesis of polypeptides, fusion proteins and CD4-targeted IL-15v molecules, for example, by chemical synthesis or by recombinant expression techniques.

Methods of making monospecific antibodies are known. Methods of making bispecific antibodies are known and described, for example, in PCT Publ. Nos. WO2011/038290; WO2012/158818, WO2012/162067, WO2015/104346, WO2016/086189, WO2016/182751, WO2017/009442, WO2017/125897, WO2017/136659, WO2017/157305, WO2017/201493, WO2018/183139, WO2018/191438, WO2019/034580, WO2019/078697 and WO2019/143636; U.S. Pat. Nos. 5,731,168; 5,807,706; 5,821,333; and U.S. Appl. Publ. Nos. 2003/020734, 2002/0155537, 2014/242079, 2015/133640, 2016/297885 and 2017/037130. Bispecific tetravalent antibodies, and methods of making them are described, e.g., in WO 02/096948 and WO 00/44788, the disclosures of both of which are herein incorporated by reference in its entirety. In addition, other publications relating to making bispecific antibodies include WO 91/00360; WO 92/08802; WO 92/05793, and WO 93/17715; Tutt et al., J. Immunol. 147:60-69 (1991); U.S. Pat. Nos. 4,474,893; 4,714,681; 4,925,648; 5,573,920; 5,601,819, 9,212,230 and 9,701,759; and Kostelny et al., J. Immunol. 148:1547-1553 (1992).

Moreover, methods of producing targeted cytokines are known and can be applied to the herein described CD4-targeted IL-15v molecules. For example, targeted IL-15v molecules are described, e.g., in WO 2023/239228 (TIGIT-targeted IL-15v); WO 2023/239226 (TIGIT-targeted IL-15v); WO 2023/196905 (LAG-3-targeted Il-15v); WO 2023/043955 (B7-H3-targeted IL-15v); WO 2022/251705 (PD-L1-targeted IL-15v); WO 2022/159771 (PD-L1-targeted IL-15v); WO 2022/140701 (ICOS-targeted IL-15v); WO 2021/072298 (PD-1-targeted IL-15v); WO 2020/132646 (NKG2D-targeted IL-15v); WO 2020/077276 (PD-1-targeted IL-15v); WO 2019/204646 (LAG-3-targeted IL-15v); WO 2019/204665 (PD-1-targeted IL-15v); WO 2019/204655 (TIM3-targeted IL-15v); WO 2018/184964 (PD-1-targeted IL-15v); and WO 2018/071918 (PD-1-targeted IL-15v).

In various embodiments, the IL-15v, the IL-15Rα Sushi domain-IL-15v fusion protein or the CD4-targeted IL-15v molecule expressed from the expression cassettes, described herein, may be produced in prokaryotic cells (e.g., bacterial cells, e.g., E. coli cells), eukaryotic cells, e.g., yeast cells, plant cells, mammalian cells, e.g., CHO-based or CHO-originated cells. The IL-15v, the IL-15Rα Sushi domain-IL-15v fusion protein or the CD4-targeted IL-15v molecule can also be produced in eukaryotic cells such as transformed cell lines (e.g., 293E, 293T, Expi293™, COS, NIH3T3). In addition, the IL-15v, the IL-15Rα Sushi domain-IL-15v fusion protein or the CD4-targeted IL-15v molecule described herein can be expressed in a yeast cell such as Pichia (see, e.g., Powers et al., J Immunol Methods. 251:123-35 (2001)), Hanseula, or Saccharomyces. In one embodiment, the IL-15v, the IL-15Rα Sushi domain-IL-15v fusion protein or the CD4-targeted IL-15v molecule described herein are produced in a CHO-based or CHO-originated cell line (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines) or a HEK293 (e.g., Expi293™) cell line. To produce the IL-15v, the IL-15Rα Sushi domain-IL-15v fusion protein or the CD4-targeted IL-15v molecule, one or more polynucleotides encoding the IL-15v, the IL-15Rα Sushi domain-IL-15v fusion protein or the CD4-targeted IL-15v molecule are constructed, introduced into an expression vector, and then expressed in one or more suitable host cells. In some embodiments, three polynucleotides encoding an IL-15Rα Sushi domain-IL-15v-Fc fusion protein, a Fab heavy chain and a Fab light chain comprising the CD4-binding domain are co-expressed in a single host cell. Standard molecular biology techniques can be used to prepare the recombinant expression vector, transfect the host cells, select for transfectants, culture the transfected cells, and recover the IL-15v, the IL-15Rα Sushi domain-IL-15v fusion protein or the CD4-targeted IL-15v molecules. As appropriate or desired, the cell or population of cells are cultured in a culture volume of at least 2 L, e.g., at least 5 L, 10 L, 50L, 100 L, 150 L, 200 L, 250 L, or more.

If the IL-15v, the IL-15Rα Sushi domain-IL-15v fusion protein or the CD4-targeted IL-15v molecule is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion of the IL-15v, IL-15Rα Sushi domain-IL-15v fusion protein or CD4-targeted IL-15v molecule. For production into the periplasm of E. coli, the pelB signal sequence (Lei et al., J. Bacteriol., 169:4379 (1987)) may be used as the signal sequence for secretion of the IL-15v, IL-15Rα Sushi domain-IL-15v fusion protein or CD4-targeted IL-15v molecule. For bacterial expression, calcium chloride methods or electroporation methods may be used to introduce the expression vector into the bacterial cell.

If the IL-15v, the IL-15Rα Sushi domain-IL-15v fusion protein or the CD4-targeted IL-15v molecule is to be expressed in mammalian host cells, e.g., such as CHO-based or CHO-originated cells, COS, and NIH3T3 cells, the expression vector includes a promoter useful for expression in these cells. In various embodiments, the promoter for expression of the IL-15v, the IL-15Rα Sushi domain-IL-15v fusion protein or the CD4-targeted IL-15v molecule in mammalian cells is a constitutive promoter or an inducible promoter. Illustrative promoters for expression of the IL-15v, the IL-15Rα Sushi domain-IL-15v fusion protein or the CD4-targeted IL-15v molecule in mammalian cells include without limitation those described above, and e.g., an SV40 promoter (Mulligan et al., Nature, 277:108 (1979)), a MMLV-LTR promoter, an EFla promoter (Mizushima et al., Nucleic Acids Res., 18:5322 (1990)), or a CMV promoter. In addition to the nucleic acid sequence encoding the IL-15v, the IL-15Rα Sushi domain-IL-15v fusion protein or the CD4-targeted IL-15v molecule thereof, the recombinant expression vectors may carry additional sequences, such as sequences that regulate replication of the vector in host cells (e.g., origins of replication) and selection marker genes. The selection marker gene facilitates selection of host cells into which the vector has been introduced (see e.g., U.S. Pat. Nos. 4,399,216, 4,634,665 and 5,179,017). In some embodiments, the selection marker gene confers resistance to drugs, such as G418, hygromycin, zeocin, blastcidin, puromycin, or methotrexate, on a host cell into which the vector has been introduced. Examples of vectors with selection markers include pMAM, pDR2, pBK-RSV, pBK-CMV, pOPRSV, pIRESbleo3, pIRESneo3, pIREShyg3, pIRESpuro3, and pOP13. In some embodiments, the selection marker is a glutamine synthetase (GS).

In one embodiment, the IL-15v, the IL-15Rα Sushi domain-IL-15v fusion protein or the CD4-targeted IL-15v molecule are produced in mammalian cells. Exemplary mammalian host cells for expressing the IL-15v, the IL-15Rα Sushi domain-IL-15v fusion protein or the CD4-targeted IL-15v molecule include Chinese Hamster Ovary (CHO cells) (including dhfr-CHO cells, e.g., described in Urlaub and Chasin (1980) Proc. Natl. Acad. Sci. USA 77:4216-4220, used with a DHFR selection marker, e.g., as described in Kaufman and Sharp (1982) Mol. Biol. 159:601 621 and glutamine synthetase (GS)-cells used with a GS selection marker, e.g., described in Lin, et al., MAbs. (2019) 11(5):965-976; and Noh, et al., Sci Rep. (2018) 8(1):5361), human embryonic kidney 293 cells (e.g., 293, 293E, 293T, Expi293™), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell. CHO and NS0 cell lines for recombinant antibody production are reviewed by Dhara, et al., BioDrugs. (2018) 32(6):571-584.

In an exemplary system for expression of the IL-15v, the IL-15Rα Sushi domain-IL-15v fusion protein or the CD4-targeted IL-15v molecule, recombinant expression vectors are introduced into dhfr-CHO cells by calcium phosphate-mediated transfection. In a specific embodiment, the dhfr-CHO cells are cells of the DG44 cell line, such as DG44i (see, e.g., Derouaz et al., Biochem Biophys Res Commun., (2006) 340(4):1069-77). Within the recombinant expression vectors, the first and second polypeptide chains are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression of the IL-15v, the IL-15Rα Sushi domain-IL-15v fusion protein or the CD4-targeted IL-15v molecule, which can be recovered from the culture medium. In one embodiment, three polypeptide chains of a CD4-targeted IL-15v molecule, as described herein, are expressed in a single cell. In one embodiment, three polypeptide chains of a CD4-targeted IL-15v, as described herein, are expressed in a single cell from a single vector. In some embodiments, the methods of production achieve at least 4 g/L, e.g., at least 5 g/L, at least 6 g/L, at least 7 g/L, at least 8 g/L, at least 9 g/L, or more, CD4-targeted IL-15v expressed or produced in a cell culture medium.

One method of making the CD4-targeted IL-15v molecules, described herein, employs so-called “knobs-into-holes” technology (Ridgway et al., Protein Eng., 9:617-621 (1996); WO 2006/028936). The mispairing problem of Ig heavy chains that is a chief drawback for making bispecific antibodies is reduced in this technology by substituting selected amino acids forming the interface of the CH3 domains in IgG. At positions within the CH3 domain at which the two heavy chains interact directly, an amino acid with a small side chain (hole) is introduced into the sequence of one heavy chain and an amino acid with a large side chain (knob) into the counterpart interacting residue location on the other heavy chain. In some instances, the CD4-targeted IL-15v molecules described herein have immunoglobulin chains in which the CH3 domains have been modified by substituting selected amino acids that interact at the interface between two polypeptides so as to preferentially form an asymmetric bispecific antigen binding molecule. In some embodiments, the CD4-targeted IL-15v molecules can be composed of immunoglobulin constant chains (e.g., Fc) of the same subclass or different subclasses. In one instance, a CD4-targeted IL-15, described herein, comprises a T366W (Eu numbering) amino acid substitution in the “knobs chain” and T366S, L368A, Y407V (Eu numbering) amino acid substitutions in the “hole chain.” In certain embodiments, an additional interchain disulfide bridge is introduced between the CH3 domains by, e.g., introducing a Y349C amino acid substitution into the “knobs chain” and a E356C amino acid substitution or a S354C amino acid substitution into the “hole chain.” In certain embodiments, R409D, K370E amino acid substitutions are introduced in the “knobs chain” and D399K, E357K amino acid substitutions in the “hole chain.” In other embodiments, Y349C, T366W amino acid substitutions are introduced in one of the chains and E356C, T366S, L368A, Y407V amino acid substitutions in the counterpart chain. In some embodiments. Y349C, T366W amino acid substitutions are introduced in one chain and S354C, T366S, L368A, Y407V amino acid substitutions in the counterpart chain. In some embodiments, Y349C, T366W amino acid substitutions are introduced in one chain and S354C, T366S, L368A, Y407V amino acid substitutions in the counterpart chain. In yet other embodiments, Y349C, T366W amino acid substitutions are introduced in one chain and S354C, T366S, L368A, Y407V amino acid substitutions in the counterpart chain (all Eu numbering).

The IL-15v, the IL-15Rα Sushi domain-IL-15v fusion protein or the CD4-targeted IL-15v molecules can be isolated from inside or outside (such as medium) of the host cell and purified as substantially pure and homogenous, non-aggregated molecules. In various embodiments, IL-15v, the IL-15Rα Sushi domain-IL-15v fusion protein or the CD4-targeted IL-15v molecules described herein, and/or the polynucleotides encoding such polypeptides, are provided in provided in isolated form. This means that such the polypeptide or polynucleotide is at least 50% w/w pure of interfering proteins, cellular and other contaminants arising from its production or purification but does not exclude the possibility that the agent is combined with an excess of pharmaceutical acceptable carrier(s) or other vehicle intended to facilitate its use. The term “isolated,” when applied to a polypeptide or polynucleotide, as described herein, denotes that the polypeptide or polynucleotide is essentially free of cellular components with which it is associated in the natural state. It can be, for example, in a homogeneous state and may be in either a dry or aqueous solution. Purity and homogeneity can be determined using known methods, e.g., analytical chemistry techniques such as polyacrylamide gel electrophoresis, column chromatography, thin layer chromatography, or high-performance liquid chromatography (HPLC) analysis. A protein that is the predominant species present in a preparation is substantially purified. An “isolated” or “purified” polypeptide or polynucleotide is substantially free of other cellular material, or culture medium when produced by recombinant techniques, or chemical precursors or other chemicals when chemically synthesized. In various embodiments, purified polypeptides and/or polynucleotides are at least 60%, 70%, 75%, 80%, 85%, 90%, 91%, 92%, 93%, 94%, 95%, 96%, 97%, 98%, 99%, or 100% (w/w), separated from, purified of, or free of interfering proteins and contaminants from production or purification. As appropriate or desired, the IL-15v, IL-15Rα Sushi domain-IL-15v fusion protein or the CD4-targeted IL-15v molecule, described herein, is the predominant macromolecular species remaining after its purification.

Methods for isolation and purification commonly used for antibody purification may be used for the isolation and purification of the herein described IL-15v, IL-15Rα Sushi domain-IL-15v fusion proteins or the CD4-targeted IL-15v molecules, and are not limited to any particular method. The IL-15v, IL-15Rα Sushi domain-IL-15v fusion proteins or the CD4-targeted IL-15v molecules may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include Protein A column and protein G column. Examples of columns using Protein A column include Hyper D, POROS, and Sepharose FF (GE Healthcare Biosciences). The present disclosure also includes IL-15v, the IL-15Rα Sushi domain-IL-15v fusion proteins and CD4-targeted IL-15v molecules that are highly purified using these purification methods. In various embodiments, the isolating or purifying step comprises Protein A chromatography, and at least 90%, 91%, 92%, 93%, 94%, 95%, 96%, 97%, 98%, 99%, or more, of the IL-15v, IL-15Rα Sushi domain-IL-15v fusion proteins or the CD4-targeted IL-15v molecules are isolated or purified. In various embodiments, the isolating or purifying step comprises Protein A chromatography, followed by ion exchange chromatography, and at least 95%, 96%, 97%, 98%, 99%, or more, of the IL-15v, IL-15Rα Sushi domain-IL-15v fusion proteins or CD4-targeted IL-15v molecules are isolated or purified. In various embodiments, at least 95%, 96%, 97%, 98%, 99%, or more, of the IL-15v, IL-15Rα Sushi domain-IL-15v fusion proteins or the CD4-targeted IL-15v molecules isolate or purify as non-aggregated soluble heterodimer as determined using size exclusion chromatography (SEC). In some embodiments, the isolated or purified IL-15v, IL-15Rα Sushi domain-IL-15v fusion proteins or CD4-targeted IL-15v molecules have increased homogeneity as assessed by analytical ion exchange chromatography, wherein the integrated area of a main peak representing an unmodified target species is at least 95%, 96%, 97%, 98%, or more, of the sum of all integrated protein peak areas. In some embodiments, the isolated or purified antigen binding molecules have fewer than 35%, 30%, 25%, 20%, 15%, 10%, 9%, 8%, 7%, or fewer, acidic contaminants.

### 10. Pharmaceutical Compositions/Formulation

Further provided are pharmaceutical compositions comprising an IL-15v, an IL-15Rα Sushi domain-IL-15v fusion protein or a CD4-targeted IL-15v molecule, as described herein, or one or more polynucleotide encoding an IL-15v, an IL-15Rα Sushi domain-IL-15v fusion protein or a CD4-targeted IL-15v molecule, as described herein, and a pharmaceutically acceptable diluent, carrier or excipient. In certain embodiments, the pharmaceutical composition comprises a therapeutically effective amount of one or more unitary doses of an IL-15v, an IL-15Rα Sushi domain-IL-15v fusion protein or a CD4-targeted IL-15v molecule, or one or more polynucleotides encoding the IL-15v, the IL-15Rα Sushi domain-IL-15v fusion protein or the CD4-targeted IL-15v molecule.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described, e.g., in Remington: The Science and Practice of Pharmacy 20th Ed. (Lippincott, Williams & Wilkins 2003); Loyd V. Allen Jr (Editor), “Remington: The Science and Practice of Pharmacy,” 22^{nd} Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing; and Shire, “Monoclonal Antibodies: Meeting the Challenges in Manufacturing, Formulation, Delivery and Stability of Final Drug Product, 2015, Woodhead Publishing.

In some embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; 2-Amino-2-(hydroxymethyl) propane-1,3-diol (i.e., tris(hydroxymethyl)aminomethane; Tris) buffers, amino acids (e.g., charged amino acids, including without limitation, aspartate, asparagine, glutamate, glutamine, histidine, lysine, arginine); and other non-toxic compatible substances employed in pharmaceutical formulations. Wetting agents, emulsifiers and lubricants, such as sodium lauryl sulfate, a polysorbate (e.g., polysorbate 20, polysorbate 40, polysorbate 60, polysorbate 80) and magnesium stearate, as well as coloring agents, release agents, coating agents, sweetening, flavoring and perfuming agents, preservatives and antioxidants can also be present in the compositions. In certain embodiments, pharmaceutical compositions are sterile. In certain embodiments, the pharmaceutical composition has a pH in the range of 4.5 to 8.5, 5.5 to 7.4, 4.5 to 6.5, 6.4 to 7.0, 6.5 to 8.5, 7.2 to 7.8, or a pH of 5.0, 5.5, 5.6, 5.7, 5.8, 5.9, 6.0, 6.5, 7.0, 7.5, 8.0 or 8.5. In one embodiment, the pharmaceutical composition comprises a physiologically acceptable buffer, pH 5.5 to 8.5, e.g., pH 5.5 to 6.5. In one embodiment, the pharmaceutical composition has an osmolarity in the range of 240-260 or 250-330 mOsmol/L. In certain embodiments, the pharmaceutical composition is isotonic or near isotonic.

The formulation of and delivery methods of pharmaceutical compositions will generally be adapted according to the site and the disease to be treated. Exemplary formulations include, but are not limited to, those suitable for parenteral administration, e.g., intravenous, intra-arterial, intramuscular, or subcutaneous administration, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays.

In some embodiments, the pharmaceutical compositions are formulated for parenteral, e.g., intravenous, subcutaneous, or intramuscular). In particular embodiments, for parenteral administration, the antibodies or antigen-binding fragments thereof are formulated in a unit dosage injectable form (solution, suspension, emulsion) in association with a pharmaceutically acceptable, parenteral vehicle. Examples of such vehicles include water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. In certain embodiments, the IL-15v, IL-15Rα Sushi domain-IL-15v fusion proteins or CD4-targeted IL-15v molecules are formulated in such vehicles at concentrations of from 5 mg/ml to 150 mg/ml.

### 11. Methods of Use

### Treating and Preventing HIV—HIV Reservoir Activation

Provided are methods for treating or preventing an HIV infection or a related disease or disorder in a subject in need thereof (e.g., a human subject), comprising providing to the subject an effective amount of an IL-15v, an IL-15Rα Sushi domain-IL-15v fusion protein or CD4-targeted IL-15v molecule, described herein, or one or more polynucleotides encoding the IL-15v, IL-15Rα Sushi domain-IL-15v fusion protein or CD4-targeted IL-15v molecule. Further provided are methods for reducing or eliminating an HIV viral reservoir in a subject in need thereof (e.g., a human subject), comprising providing to the subject an effective amount of an IL-15v, an IL-15Rα Sushi domain-IL-15v fusion protein or CD4-targeted IL-15v molecule, described herein, or one or more polynucleotides encoding the IL-15v, IL-15Rα Sushi domain-IL-15v fusion protein or CD4-targeted IL-15v molecule. In some embodiments, the methods entail co-administering a second therapeutic agent, e.g., a fusion protein comprising a CD4 extracellular (EC) domain, e.g., a CD4-Fc fusion protein, having one or both of CD4 domains D1 and D2 or variants thereof (e.g., as described in in WO 2011/146891, WO 2019/183387, WO 2022/046644 (e.g., GS 8588) and WO 2024/094690), an anti-HIV broadly neutralizing antibody, a further latency reversal agent (LRA), an anti-HIV vaccine, or combinations thereof.

The term “treatment” or “treating,” to the extent it relates to a disease or condition, includes one or more of preventing the disease or condition from occurring, inhibiting the disease or condition, eliminating the disease or condition, and relieving one or more symptoms of the disease or condition. The terms “treating” and “treatment” as used herein are intended to mean the administration of a compound or composition according to the embodiments disclosed herein to at least one of alleviate or eliminate symptoms of HIV infection and to reduce viral load in a patient. As used herein, the term “effective amount” in the context of the administration of a therapy to a subject refers to the amount of a therapy that achieves a desired prophylactic or therapeutic effect. In various embodiments, the polynucleotide or polynucleotides may be present in a lipid nanoparticle (LNP) or a vector, e.g., a plasmid vector or a viral vector. In some embodiments, the related disease or disorder is caused by infection with HIV. In other embodiments, it is acquired immune deficiency syndrome (AIDS). In certain embodiments, the subject is a virologically suppressed HIV-infected mammal, while in other embodiments, the subject is a treatment-naïve HIV-infected mammal. In certain embodiments, a treatment-naïve subject has a viral load between 10³ and 10⁵ copies/ml, and in certain embodiments, a virologically suppressed subject has a viral load of copies/ml in blood of less than 500, e.g., less than 400, less than 300, less than 200, less than 100, less than 50. In another embodiment, the subject is a mammal, e.g., a human. In certain embodiments, the subject has been diagnosed with an HIV, e.g., HIV-1 or HIV-2, infection or a related disease or disorder, e.g., AIDS, or is considered at risk for developing an HIV, e.g., HIV-1 or HIV-2, infection or a related disease or disorder, e.g., AIDS. Subjects at risk for HIV-related diseases or disorders include patients who have come into contact with an infected person or who have been exposed to HIV in some other way. Administration of a prophylactic agent can occur prior to the manifestation of symptoms characteristic of HIV-related disease or disorder, such that a disease or disorder is prevented or, alternatively, delayed in its progression.

Also provided are methods for preventing or inhibiting an increase in HIV virus titer, virus replication, virus proliferation or an amount of an HIV viral DNA, HIV proviral DNA, or HIV viral protein in a subject (e.g., a human subject). In one embodiment, the method comprises providing to the subject in need thereof an amount of an IL-15v, an IL-15Rα Sushi domain-IL-15v fusion protein or a CD4-targeted IL-15v molecule, described herein, or one or more polynucleotides encoding an IL-15v, an IL-15Rα Sushi domain-IL-15v fusion protein or a CD4-targeted IL-15v molecule, described herein, effective to prevent, reduce or inhibit an increase in HIV titer, virus replication, virus proliferation, or an amount of an HIV protein of one or more HIV strains or isolates in the subject. In certain embodiments, the method further comprises measuring an amount of HIV viral or proviral DNA or protein at one or more time points, e.g., before and after the subject in provided with an antibody or antibodies of the present disclosure. Methods and biomarkers for determining an amount of HIV viral or proviral DNA or protein in a subject are known and available in the art, and described for example, in Siliciano, J. D. et al., Curr Opin. HIV AIDS, 5(6):491-7 (2010), and Rouzioux, C. et al., Curr Opin HIV AIDS, 8(3):170-5 (2013). In various embodiments, the human subject is an adult, a juvenile or an infant. The subject may be symptomatic (e.g., viremic) or asymptomatic (e.g., acutely infected or ART suppressed). In some embodiments, the human subject is acutely infected or recently infected with HIV. In certain embodiments, the subject has not seroconverted. In some embodiments, the human subject is chronically infected with HIV. The subject many or may not be receiving a regimen of antiretroviral therapy (ART). In some embodiments, the human subject is heavily treatment experienced (HTE) (e.g., someone who has had virologic failure or rebound on multiple treatment regimens and has limited options for ART).

Patients can be categorized into Fiebig stages I-VI, which are based on a sequential gain in positive HIV-1 clinical diagnostic assays (viral RNA measured by PCR, p24 and p31 viral antigens measured by enzyme-linked immunosorbent assay (ELISA). p24 antigen is a viral core protein that transiently appears in the blood during the ramp-up phase once HIV-1 RNA levels rise above 10,000 copies/mL and before the development of detectable HIV antibodies. In Fiebig stage I, during ramp-up viremia, only HIV-1 RNA in the blood can be detected. Fiebig stage II commences about 7 days later, when results of tests to detect p24 antigen become positive. In Fiebig stage III, within about 5 days after p24 antigen test results become positive, IgM anti-HIV-1 antibodies can be detected with sufficiently sensitive enzyme immunoassays (EIAs) (e.g., third-generation EIAs). Stage III typically occurs 1-2 weeks after the onset of acute retroviral symptoms. Fiebig stage IV represents the development of an indeterminate Western blot test and occurs about 3 days after EIA tests show positive results. Conversion to a clearly positive Western blot test, Fiebig stage V, generally occurs after another 7 days, or about 1 month after initial infection. Fiebig stages of HIV infection are described, e.g., in Fiebig, et al., AIDS. (2003) 17(13):1871-9; Cohen, et al., J Infect Dis. (2010) 202 Suppl 2:S270-7; and McMichael, et al., Nature Reviews Immunology (2010) 10:11-23, which are hereby incorporated herein by reference in their entireties for all purposes. In some embodiments, the IL-15v, IL-15Rα Sushi domain-IL-15v fusion protein or CD4-targeted IL-15v molecule, described herein, or one or more polynucleotides encoding the IL-15v, IL-15Rα Sushi domain-IL-15v fusion protein or CD4-targeted IL-15v molecule, described herein, is administered to a human subject having an HIV infection of Fiebig stage IV or earlier, e.g., Fiebig stage I, Fiebig stage II, Fiebig stage III or Fiebig stage IV. In some embodiments, the biological sample evaluated is from a human subject having an HIV infection of an HIV infection of Fiebig stage V or Fiebig stage VI.

For in vivo treatment of mammalian subject, e.g., humans, the subject may be administered or provided a pharmaceutical composition comprising an IL-15v, an IL-15Rα Sushi domain-IL-15v fusion protein or CD4-targeted IL-15v molecule, described herein, and optionally an additional anti-HIV therapeutic agent (e.g., a CD4 EC domain-Fc fusion protein, an anti-HIV broadly neutralizing antibody (bNAb), an HIV vaccine). When used for in vivo therapy, an antibody or antibodies described herein are typically administered or provided to the patient in therapeutically effective amounts (i.e., amounts that eliminate or reduce at least one of the patient's viral burden and viral reservoir). Amounts effective for this use depend on the severity and course of the disease or condition, previous therapy, the patient's health status, weight, and response to the IL-15v, the IL-15Rα Sushi domain-IL-15v fusion protein or the CD4-targeted IL-15v molecule, described herein, and the judgment of the treating physician. Therapeutically effective amounts are optionally determined by methods including, but not limited to, a dose escalation and/or dose ranging clinical trial. The amount of a administered IL-15v, IL-15Rα Sushi domain-IL-15v fusion protein or CD4-targeted IL-15v molecule, described herein that corresponds to such an amount varies depending upon factors such as the particular IL-15v, IL-15Rα Sushi domain-IL-15v fusion protein or CD4-targeted IL-15v molecule, described herein, inflammatory disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated. In some embodiments, an IL-15v, an IL-15Rα Sushi domain-IL-15v fusion protein or a CD4-targeted IL-15v molecule, described herein, is administered at a dose of from 0.5 μg/kg to 10 mg/kg body weight per administration. In some embodiments, dosing of an IL-15v, an IL-15Rα Sushi domain-IL-15v fusion protein or a CD4-targeted IL-15v molecule, described herein, can be reduced or decreased when combining with a second therapeutic agent, e.g., at least one of a CD4 EC domain-Fc fusion protein, an anti-HIV broadly neutralizing antibody (bNAb), an HIV vaccine.

In some embodiments, the IL-15v, IL-15Rα Sushi domain-IL-15v fusion protein or CD4-targeted IL-15v have a serum half-life in a human of less than 3 days, less than 2 days or less than 1 day. In various embodiments, the IL-15v, IL-15Rα Sushi domain-IL-15v fusion protein or CD4-targeted IL-15v molecule, described herein, has a serum half-life in a human or cynomolgus monkey of less than 24 hours, e.g., less than 20 hours, less than 18 hours, less than 16 hours, or shorter, but long enough for efficacious exposure, e.g., at least 3 hours. In various embodiments, the IL-15v, IL-15Rα Sushi domain-IL-15v fusion protein or CD4-targeted IL-15v molecule, described herein, has a first antigen binding domain that binds to CD4 with a binding equilibrium dissociation constant (K_{D}) of lower than 5 nM, e.g., lower than 4 nM, 3 nM, 2 nM, 1 nM, or lower, and has a serum half-life in a human or cynomolgus monkey of at less than 24 hours, or shorter, but long enough for efficacious exposure, e.g., at least 3 hours. In some embodiments the IL-15v, IL-15Rα Sushi domain-IL-15v fusion protein or CD4-targeted IL-15v molecule has a first antigen binding domain that binds to CD4 with a K_{D} of lower than 3 nM (e.g., 2 nM or 1 nM or lower) and antigen binding molecule has a serum half-life in a human or cynomolgus monkey of less than 24 hours.

In various embodiments, the IL-15v, IL-15Rα Sushi domain-IL-15v fusion protein or CD4-targeted IL-15v molecule, described herein, is administered one or multiple times. In embodiments employing multiple administration regimens, as appropriate the IL-15v, IL-15Rα Sushi domain-IL-15v fusion protein or CD4-targeted IL-15v molecule, described herein, can be administered in once daily, once weekly (i.e., QW), once bi-weekly (i.e. once every other week, or once every two weeks or Q2W), once every three weeks (i.e., Q3W), once monthly (i.e., QM or Q4W) or once bi-monthly (i.e. once every other month, or once every two months or Q2M or Q8W) dosing or administration intervals. In some embodiments, the IL-15v, IL-15Rα Sushi domain-IL-15v fusion protein or CD4-targeted IL-15v molecule, described herein, are administered intravenously, subcutaneously or intramuscularly weekly (QW). In some embodiments, the IL-15v, IL-15Rα Sushi domain-IL-15v fusion protein or CD4-targeted IL-15v molecule, described herein, are administered intravenously, subcutaneously or intramuscularly bi-weekly (i.e. once every other week, or once every two weeks or Q2W). In some embodiments, the IL-15v, IL-15Rα Sushi domain-IL-15v fusion protein or CD4-targeted IL-15v molecule, described herein, are administered intravenously, subcutaneously or intramuscularly once every three weeks (Q3W). In some embodiments, the IL-15v, IL-15Rα Sushi domain-IL-15v fusion protein or CD4-targeted IL-15v molecule, described herein, are administered intravenously, subcutaneously or intramuscularly monthly (QM or Q4W). In some embodiments, the IL-15v, IL-15Rα Sushi domain-IL-15v fusion protein or CD4-targeted IL-15v molecule, described herein, are administered intravenously, subcutaneously or intramuscularly. As appropriate, doses administered in multiple administration regimens can be the same or different between the first and subsequent doses of the IL-15v, IL-15Rα Sushi domain-IL-15v fusion protein or CD4-targeted IL-15v molecule. As appropriate, doses administered in multiple administration regimens can be the same or different between the IL-15v, IL-15Rα Sushi domain-IL-15v fusion protein or CD4-targeted IL-15v molecule, described herein, and a second therapeutic agent (e.g., at least one of a CD4 EC domain-Fc fusion protein, an anti-HIV broadly neutralizing antibody (bNAb), an HIV vaccine).

As appropriate, the IL-15v, IL-15Rα Sushi domain-IL-15v fusion protein or CD4-targeted IL-15v molecule, described herein, are administered or provided to a mammalian subject, e.g., a human, in accordance with known methods, such as, but not limited to, intravenous administration, e.g., as a bolus or by continuous infusion over a period of time, by intramuscular, subcutaneous, intraperitoneal, intracerebrospinal, intraarticular, intrasynovial, intrathecal, oral, topical, or inhalation routes. As appropriate, the IL-15v, IL-15Rα Sushi domain-IL-15v fusion protein or CD4-targeted IL-15v molecule, described herein, may be administered parenterally, when possible, at the target cell site, or intravenously. In one embodiment, administration of the IL-15v, IL-15Rα Sushi domain-IL-15v fusion protein or CD4-targeted IL-15v molecule, described herein, to the subject is via an intravenous route. In another embodiment, administration of the IL-15v, IL-15Rα Sushi domain-IL-15v fusion protein or CD4-targeted IL-15v molecule, described herein, to the subject is via a subcutaneous route. In another embodiment, administration of the IL-15v, IL-15Rα Sushi domain-IL-15v fusion protein or CD4-targeted IL-15v molecule, described herein, to the subject is via an intramuscular route. In various embodiments, polynucleotides encoding the IL-15v, IL-15Rα Sushi domain-IL-15v fusion protein or CD4-targeted IL-15v molecule, described herein, can be electroporated, e.g., for transdermal delivery. In some embodiments, pharmaceutical compositions of the disclosure are administered to a subject systemically, parenterally, or locally.

Further provided are methods for treating an HIV infection, comprising administering to a human subject in need thereof a therapeutically effective amount of the IL-15v, IL-15Rα Sushi domain-IL-15v fusion protein or CD4-targeted IL-15v molecule, described herein. In some embodiments, the present disclosure provides a method for preventing an HIV infection, comprising administering to a human subject in need thereof a therapeutically effective amount of the IL-15v, IL-15Rα Sushi domain-IL-15v fusion protein or CD4-targeted IL-15v molecule, described herein.

In one embodiment, a method for treating an HIV infection in a human subject having or at risk of having the infection is provided, the method comprising administering to the human subject a therapeutically effective amount of the IL-15v, IL-15Rα Sushi domain-IL-15v fusion protein or CD4-targeted IL-15v molecule, described herein, optionally in combination with a therapeutically effective amount of one or more (e.g., one, two, three, one or two, or one to three) additional therapeutic agents. In some embodiments, after one or more administrations of the IL-15v, IL-15Rα Sushi domain-IL-15v fusion protein or CD4-targeted IL-15v molecule, described herein, optionally with one or more additional therapeutic agents, the subject does not exhibit symptoms of HIV or AIDS in the absence of anti-retroviral treatment (ART) for at least 6 months, at least 1 year, at least 2 years, at least 3 years, or more. In some embodiments, after one or more administrations of the IL-15v, IL-15Rα Sushi domain-IL-15v fusion protein or CD4-targeted IL-15v molecule, described herein, optionally with one or more additional therapeutic agents, the subject has a viral load of copies/ml blood of less than 500, e.g., less than 400, less than 300, less than 200, less than 100, less than 50, in the absence of anti-retroviral treatment (ART) for at least 6 months, at least 1 year, at least 2 years, at least 3 years, or more.

### Treating and Preventing Hepatitis B Virus

Further provided are methods for eliciting and/or enhancing an immune response to human hepatitis B virus (HBV) in a subject in need thereof. Also provided are methods of treating or preventing human hepatitis B virus (HBV) in a subject in need thereof. Also provided are methods of inhibiting HBV replication in an infected individual. Further provided are methods for reducing the viral load associated with HBV infection. In various embodiments, the methods comprise administering to the subject an effective amount of the IL-15v, IL-15Rα Sushi domain-IL-15v fusion protein or CD4-targeted IL-15v, as described herein.

In another aspect, provided are IL-15v, IL-15Rα Sushi domain-IL-15v fusion proteins and CD4-targeted IL-15v, as described herein, polynucleotides encoding such polypeptides, vectors, a lipoplex (e.g., LNP), and compositions comprising such polypeptides or polynucleotides, as described herein, for use in eliciting and/or enhancing an immune response to human hepatitis B virus (HBV) in a subject in need thereof. Further provided are IL-15v, IL-15Rα Sushi domain-IL-15v fusion proteins and CD4-targeted IL-15v, as described herein, polynucleotides encoding such polypeptides, vectors, a lipoplex (e.g., LNP), and compositions comprising such polypeptides or polynucleotides, as described herein, for use in inhibiting HBV replication in an infected individual and/or for reducing the viral load associated with HBV infection.

In various embodiments, the subject is infected with HBV, is suspected of being infected with HBV, or is at risk of being infected with HBV. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). In various embodiments, the subject is chronically infected with HBV, e.g., infected with HBV for longer than 6 months. Typically, the individual is suffering from a chronic hepatitis B infection, although it is within the scope of the present disclosure to treat people who are acutely infected with HBV. Accordingly, in some embodiments, the subject is acutely infected with HBV. In some embodiments, the subject is co-infected with hepatitis D virus (HDV).

In various embodiments, the subject may be asymptomatic. In some embodiments, the subject is experiencing or exhibiting symptoms associated with HBV infection. Symptoms of HBV can include, e.g., jaundice, visible webs of swollen blood vessels in the skin, dark-colored (e.g., orange or brown) urine, light-colored feces, fever, persistent fatigue, malaise, abdominal pain, abdominal fluid, loss of appetite, nausea, and vomiting. Chronic infection with HBV can lead to one or more symptoms including, e.g., hepatic failure, hepatic cancer, hepatic fibrosis and hepatic cirrhosis. One or more administrations of an anti-PD-1 binding domain, an anti-PD-1 antibody, a multi-specific binding molecule comprising an anti-PD-1 binding domain and a second antigen binding domain, described herein, polynucleotides encoding such polypeptides, vectors, a lipoplex (e.g., LNP), and compositions comprising such polypeptides or polynucleotides, as described herein, can prevent, delay, alleviate, mitigate, inhibit, reverse or eliminate one or more symptoms associated with or caused by HBV infection.

As appropriate, a subject can be treated with multiple administrations over a time period of at least 2 weeks to 3 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or longer, or until sAg is no longer detectable in the serum or plasma of the subject.

In some embodiments, after one or more administrations of the IL-15v, IL-15Rα Sushi domain-IL-15v fusion protein or CD4-targeted IL-15v, as described herein, polynucleotides encoding such polypeptides, vectors, a lipoplex (e.g., LNP), and compositions comprising such polypeptides or polynucleotides, as described herein, optionally with one or more additional therapeutic agents, described herein, the subject does not exhibit symptoms of HBV in the absence of antiviral treatment for at least 6 months, at least 1 year, at least 2 years, at least 3 years, or more. In some embodiments, after one or more administrations of the IL-15v, IL-15Rα Sushi domain-IL-15v fusion protein or CD4-targeted IL-15v, as described herein, polynucleotides encoding such polypeptides, vectors, a lipoplex (e.g., LNP), and compositions comprising such polypeptides or polynucleotides, as described herein, optionally with one or more additional therapeutic agents, described herein, sAg is no longer detectable in the serum or plasma of the subject, in the absence of antiviral treatment for at least 6 months, e.g., at least 1 year, at least 2 years, at least 3 years, or more.

### Treating and Preventing Cancer

In various embodiments, the IL-15v, IL-15Rα Sushi domain-IL-15v fusion protein or CD4-targeted IL-15v, as described herein, polynucleotides encoding such polypeptides, vectors, a lipoplex (e.g., LNP), and compositions comprising such polypeptides or polynucleotides can be used to treat, mitigate, ameliorate, and/or delay the progression of cancer, e.g., where administration of IL-15v, IL-15Rα Sushi domain-IL-15v fusion protein targeted IL-15v, has been shown to induce a durable response and antitumor activity.

Accordingly, in some embodiments, provided are methods of preventing, reducing and/or inhibiting the recurrence, growth, proliferation, migration and/or metastasis of a cancer cell or population of cancer cells in a subject in need thereof. Further provided are methods of enhancing, improving, and/or increasing the response to an anticancer therapy in a subject in need thereof. In some embodiments, the methods entail administering to the subject an effective amount of an IL-15v, an IL-15Rα Sushi domain-IL-15v fusion protein or a CD4-targeted IL-15v, as described herein, polynucleotides encoding such polypeptides, vectors, a lipoplex (e.g., LNP), and compositions comprising such polypeptides or polynucleotides, as described herein.

In another aspect, provided are IL-15v, IL-15Rα Sushi domain-IL-15v fusion proteins and CD4-targeted IL-15v, as described herein, polynucleotides encoding such polypeptides, vectors, a lipoplex (e.g., LNP), and compositions comprising such polypeptides or polynucleotides, as described herein, for use in preventing, reducing and/or inhibiting the recurrence, growth, proliferation, migration and/or metastasis of a cancer cell or population of cancer cells in a subject in need thereof. Further provided are IL-15v, IL-15Rα Sushi domain-IL-15v fusion proteins and CD4-targeted IL-15v, as described herein, polynucleotides encoding such polypeptides, vectors, a lipoplex (e.g., LNP), and compositions comprising such polypeptides or polynucleotides, as described herein, for use in enhancing, improving, and/or increasing the response to an anticancer therapy in a subject in need thereof.

As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

In some embodiments, the subject may be a human who exhibits one or more symptoms associated with cancer or hyperproliferative disease (e.g., a tumor). In some embodiments, the subject may be a human who exhibits one or more symptoms associated with cancer. Any of the methods of cancer treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. One or more administrations of the IL-15v, IL-15Rα Sushi domain-IL-15v fusion protein or CD4-targeted IL-15v, as described herein, polynucleotides encoding such polypeptides, vectors, a lipoplex (e.g., LNP), and compositions comprising such polypeptides or polynucleotides, as described herein, optionally with one or more additional therapeutic agents, can promote reduction or retraction of a tumor, decrease or inhibit tumor growth or cancer cell proliferation, and/or induce, increase or promote tumor cell killing. In some embodiments, the subject is in cancer remission. One or more administrations of the IL-15v, IL-15Rα Sushi domain-IL-15v fusion protein or CD4-targeted IL-15v, as described herein, polynucleotides encoding such polypeptides, vectors, a lipoplex (e.g., LNP), and compositions comprising such polypeptides or polynucleotides, as described herein, optionally with one or more additional therapeutic agents, can prevent or delay metastasis or recurrence of cancer.

In some embodiments, the subject may be a human who is at risk, or genetically or otherwise predisposed (e.g., risk factor) to developing cancer or hyperproliferative disease who has or has not been diagnosed. In some embodiments, the subject may be a human who is at risk, or genetically or otherwise predisposed (e.g., risk factor) to a disease, disorder, or symptoms thereof, caused by a viral infection who has or has not been diagnosed.

In addition, the subject may be a human who is undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In some embodiments, a therapeutically effective amount of an IL-15v, an IL-15Rα Sushi domain-IL-15v fusion protein or a CD4-targeted IL-15v, as described herein, polynucleotides encoding such polypeptides, vectors, a lipoplex (e.g., LNP), and compositions comprising such polypeptides or polynucleotides, as described herein, optionally, with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer or myeloproliferative disease. In some embodiments, a therapeutically effective amount of an IL-15Rα Sushi domain-IL-15v fusion protein or a CD4-targeted IL-15v, as described herein, polynucleotides encoding such polypeptides, vectors, a lipoplex (e.g., LNP), and compositions comprising such polypeptides or polynucleotides, as described herein, optionally, with one or more additional therapeutic agents, as described herein, can (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

Examples of tissues containing cancerous cells whose proliferation can reduced and/or inhibited by an IL-15v, an IL-15Rα Sushi domain-IL-15v fusion protein or a CD4-targeted IL-15v, as described herein, polynucleotides encoding such polypeptides, vectors, a lipoplex (e.g., LNP), and compositions comprising such polypeptides or polynucleotides, as described herein, and against which the methods described herein are useful include but are not limited to breast, prostate, brain, blood, bone marrow, liver, pancreas, skin (melanoma), kidney, colon, ovary, lung (e.g., non-small cell lung cancer, small cell lung cancer), testicle, penis, thyroid, parathyroid, pituitary, thymus, retina, uvea, conjunctiva, spleen, head, neck (e.g., head and neck squamous cell carcinoma (HNSCC)), trachea, gall bladder, rectum, salivary gland, adrenal gland, throat, esophagus, lymph nodes, sweat glands, sebaceous glands, muscle, heart, and stomach. In some embodiments, the cancer is a leukemia, e.g., acute myeloid leukemia (AML) or myelodysplastic syndrome (MDS), or adult T cell leukemia (ATL).

In some embodiments, the subject has a solid tumor. In various embodiments, the cancer or tumor is malignant and/or metastatic. In various embodiments, the subject has a cancer selected from the group consisting of an epithelial tumor (e.g., a carcinoma, a squamous cell carcinoma, a basal cell carcinoma, a squamous intraepithelial neoplasia), a glandular tumor (e.g., an adenocarcinoma, an adenoma, an adenomyoma), a mesenchymal or soft tissue tumor (e.g., a sarcoma, a rhabdomyosarcoma, a leiomyosarcoma, a liposarcoma, a fibrosarcoma, a dermatofibrosarcoma, a neurofibrosarcoma, a fibrous histiocytoma, an angiosarcoma, an angiomyxoma, a leiomyoma, a chondroma, a chondrosarcoma, an alveolar soft-part sarcoma, an epithelioid hemangioendothelioma, a Spitz tumor, a synovial sarcoma), and a lymphoma. In some embodiments, the subject has a T cell lymphoma, e.g., a peripheral T cell lymphoma or a cutaneous T cell lymphoma (e.g., mycosis fungoides/Sezary syndrome).

### 12. Combination Therapies

In certain embodiments, this disclosure provides a method for treating or preventing an HIV infection in a human subject having, or at risk of having, the HIV infection. The method comprises administering to the human subject a therapeutically effective amount of the IL-15v, IL-15Rα Sushi domain-IL-15v fusion protein or CD4-targeted IL-15v, as described herein, or a pharmaceutical composition thereof, in combination with a therapeutically effective amount of one or more (e.g., one, two, three, one or two, or one to three) additional therapeutic agents. In one embodiment, a method for treating an HIV infection in a human subject having or at risk of having the infection is provided, the method comprising administering to the human subject a therapeutically effective amount of the IL-15v, IL-15Rα Sushi domain-IL-15v fusion protein or CD4-targeted IL-15v, as described herein, in combination with a therapeutically effective amount of one or more (e.g., one, two, three, one or two, or one to three) additional therapeutic agents.

In one embodiment, pharmaceutical compositions comprising an IL-15v, an IL-15Rα Sushi domain-IL-15v fusion protein or a CD4-targeted IL-15v, as described herein, or a pharmaceutical composition thereof, in combination with one or more (e.g., one, two, three, one or two, or one to three) additional therapeutic agents, and a pharmaceutically acceptable carrier, diluent, or excipient are provided.

In certain embodiments, provided are methods for treating an HIV infection, comprising administering to a patient in need thereof a therapeutically effective amount of the IL-15v, IL-15Rα Sushi domain-IL-15v fusion protein or CD4-targeted IL-15v, as described herein, or a pharmaceutical composition thereof, in combination with a therapeutically effective amount of one or more additional therapeutic agents which are suitable for treating an HIV infection.

In certain embodiments, one or more of the IL-15v, IL-15Rα Sushi domain-IL-15v fusion protein or CD4-targeted IL-15v, as described herein, or a pharmaceutical composition thereof, are combined or co-administered with one, two, three, four, or more additional therapeutic agents. In certain embodiments, the IL-15v, IL-15Rα Sushi domain-IL-15v fusion protein or CD4-targeted IL-15v, as described herein, or a pharmaceutical composition thereof, are combined or co-administered with two additional therapeutic agents. In other embodiments, the IL-15v, IL-15Rα Sushi domain-IL-15v fusion protein or CD4-targeted IL-15v, as described herein, or a pharmaceutical composition thereof, are combined or co-administered with three additional therapeutic agents. In further embodiments, the IL-15v, IL-15Rα Sushi domain-IL-15v fusion protein or CD4-targeted IL-15v, as described herein, or a pharmaceutical composition thereof, is combined or co-administered with four additional therapeutic agents. The one, two, three, four, or more additional therapeutic agents can be different therapeutic agents selected from the same class of therapeutic agents (e.g., one or more anti-HIV broadly neutralizing antibodies), and/or they can be selected from different classes of therapeutic agents (e.g., one or more anti-HIV broadly neutralizing antibodies and one or more TLR agonists).

### Administration of HIV Combination Therapies

In certain embodiments, the IL-15v, IL-15Rα Sushi domain-IL-15v fusion protein or CD4-targeted IL-15v, as described herein, are co-administered with one or more additional therapeutic agents. Co-administration of the IL-15v, IL-15Rα Sushi domain-IL-15v fusion protein or CD4-targeted IL-15v, as described herein, with one or more additional therapeutic agents generally refers to simultaneous or sequential administration of the IL-15v, IL-15Rα Sushi domain-IL-15v fusion protein or CD4-targeted IL-15v, as described herein, and one or more additional therapeutic agents, such that therapeutically effective amounts of the IL-15v, IL-15Rα Sushi domain-IL-15v fusion protein or CD4-targeted IL-15v, as described herein, and the one or more additional therapeutic agents are both present in the body of the patient. When administered sequentially, the combination may be administered in two or more administrations.

“Co-administration” includes concurrent administration as well as sequential administration of unit dosages of the IL-15v, IL-15Rα Sushi domain-IL-15v fusion protein or CD4-targeted IL-15v, as described herein, before or after administration of unit dosages of one or more additional therapeutic agents. For example, the IL-15v, IL-15Rα Sushi domain-IL-15v fusion protein or CD4-targeted IL-15v, as described herein, may be administered within seconds, minutes, hours or days of the administration of the one or more additional therapeutic agents. In some embodiments, a unit dose of the IL-15v, IL-15Rα Sushi domain-IL-15v fusion protein or CD4-targeted IL-15v, as described herein, is administered first, followed within seconds, minutes, hours or days by administration of a unit dose of one or more additional therapeutic agents. Alternatively, a unit dose of one or more additional therapeutic agents is administered first, followed by administration of a unit dose of the IL-15v, IL-15Rα Sushi domain-IL-15v fusion protein or CD4-targeted IL-15v, as described herein, within seconds, minutes, hours or days. In other embodiments, a unit dose of the IL-15v, IL-15Rα Sushi domain-IL-15v fusion protein or CD4-targeted IL-15v, as described herein, is administered first, followed, after a period of hours (e.g., 1-12 hours, 1-24 hours, 1-36 hours, 1-48 hours, 1-60 hours, 1-72 hours), by administration of a unit dose of one or more additional therapeutic agents. In yet other embodiments, a unit dose of one or more additional therapeutic agents is administered first, followed, after a period of hours (e.g., 1-12 hours, 1-24 hours, 1-36 hours, 1-48 hours, 1-60 hours, 1-72 hours), by administration of a unit dose of the IL-15v, IL-15Rα Sushi domain-IL-15v fusion protein or CD4-targeted IL-15v, as described herein.

In certain embodiments, the IL-15v, IL-15Rα Sushi domain-IL-15v fusion protein or CD4-targeted IL-15v, as described herein, is combined or co-administered with one or more additional therapeutic agents in a unitary dosage form for simultaneous administration to a patient, for example as a liquid or suspension dosage form, e.g., for intravenous, intramuscular or subcutaneous administration.

In certain embodiments, the IL-15v, IL-15Rα Sushi domain-IL-15v fusion protein or CD4-targeted IL-15v, as described herein, are formulated as a liquid or aqueous solution or suspension which may optionally contain one or more other additional therapeutic agents useful for treating HIV. In certain embodiments, the liquid solution or suspension can contain another active ingredient for treating HIV, as another anti-HIV antibody or antigen-binding fragment thereof, a HIV protease inhibitor, a HIV non-nucleoside or non-nucleotide inhibitor of reverse transcriptase, a HIV nucleoside or nucleotide inhibitor of reverse transcriptase, a HIV integrase inhibitor, a HIV non-catalytic site (or allosteric) integrase inhibitor, pharmacokinetic enhancer, and combinations thereof.

In certain embodiments, the IL-15v, IL-15Rα Sushi domain-IL-15v fusion protein or CD4-targeted IL-15v, as described herein, can be administered in multiple administration regimens, i.e., are administered two or more times at first and subsequent time points. In certain embodiments, such liquid solutions or suspensions are suitable for once daily, once weekly (i.e., QW), once bi-weekly (i.e. once every other week, or once every two weeks or Q2W), once every three weeks (Q3W), once monthly (i.e., QM) or once bi-monthly dosing (i.e. once every other month, or once every two months or Q2M) dosing or administration intervals. In some embodiments, the IL-15v, IL-15Rα Sushi domain-IL-15v fusion protein or CD4-targeted IL-15v, as described herein, is administered once daily, once weekly (i.e., QW), once bi-weekly (i.e. once every other week, or once every two weeks or Q2W), once every three weeks (Q3W), once monthly (i.e., QM), once bi-monthly (i.e. once every other month, or once every two months or Q2M), once every three months (i.e., Q3M), once every four months (i.e., Q4M), once every 5 months (i.e., Q5M), or once every 6 months (i.e., Q6M) dosing. In some embodiments, the IL-15v, IL-15Rα Sushi domain-IL-15v fusion protein or CD4-targeted IL-15v, as described herein, is administered intravenously, subcutaneously or intramuscularly bi-weekly (i.e. once every other week, or once every two weeks or Q2W). In some embodiments, the IL-15v, IL-15Rα Sushi domain-IL-15v fusion protein or CD4-targeted IL-15v, as described herein, is administered intravenously, subcutaneously or intramuscularly monthly (e.g., once every four weeks (Q4W) or once monthly (Q1M)).

a. Combination Therapies with One or More Broadly Neutralizing Antibodies

In certain embodiments, a IL-15v, IL-15Rα Sushi domain-IL-15v fusion protein or CD4-targeted IL-15v, as described herein, is combined or co-administered with one or more additional anti-HIV broadly neutralizing antibodies (bNAbs), or fragments thereof. The one or more additional bNAbs can be in any format (e.g., monospecific, multi-specific, IgG, scFv). Generally, the one or more additional bNAbs bind to a different antigen, or region or epitope of HIV that does not compete with the CD4-binding domain. Multiple clinical studies have now shown that treatment of HIV infected individuals with single broadly neutralizing antibodies (bNAbs) leads to temporary suppression of sensitive viruses, followed by rapid outgrowth of resistant viruses-many of which appear to be rare pre-existing viral variants.

Scheid, et al. reported that VRC01 (a V3-glycan binding bNAb) and 3BNC117 (a CD4 binding site binding bNAb) neutralized 96% of 118 cross-clade viruses tested in-vitro (Scheid et al., Science, 333:1633-1637 (2011)). Further, clinical trials showed that many HIV infected patients receiving the antibody treatment exhibited rare and pre-existing resistant clones, even when their plasma HIV isolates appeared to be sensitive to the antibody (Caskey et al., Nature, 522:487-491 (2016); Scheid et al., Nature, 535:556-560 (2016)). These results suggested that 3BNC117 may be broad when tested against HIV isolates collected from different patients (inter-patient bread), yet it may not neutralize 100% of viral isolates within individual patients (intra-patient breadth).

An antibody known as 10-1074, part of the PGT-121 lineage and taken from the same donor and with similar neutralizing breadth, has also been tested in clinical trials (Mouquet et al., PNAS, 109: E3268-3277 (2012); Caskey et al., Nature Medicine, 23:185-191 (2017)). 10-1074 was originally shown to neutralize approximately 66% of 60 viruses tested at an IC50 below 50 μg/mL (Mouquet et al., PNAS (supra)). The 10-1074 trials showed that in many patients received 10-1074 therapy, there were resistant clones, even when the plasma HIV isolates appeared to be sensitive to the antibody (Caskey et al. Nature Medicine (supra)). This data suggests that most patients may harbor rare pre-existing viral variants that are resistant to 10-1074. These 10-1074 resistance variants showed correlated cross-resistance to PGT-121, consistent with close evolutionary relationship between 10-1074 and PGT-121. However, nearly all of the resistant viruses isolated during the 10-1074 clinical trial were sensitive to neutralization by 3BNC117 (Caskey et al. Nature Medicine (supra)). This data is consistent with the conclusion that combination antibody therapy, using complementary bNAbs that bind to different regions of gp120, allows for more complete intra-patient viral coverage.

In certain embodiments, the bNAb combinations can achieve complete intra-patient viral coverage. In some embodiments, the combination therapy includes first and second antigen binding molecules, wherein the first and second antigen binding molecules bind to different first and second epitopes or regions of gp120, or gp120 and gp41. In some embodiments, the combination therapy includes an IL-15v, IL-15Rα Sushi domain-IL-15v fusion protein or CD4-targeted IL-15v, as described herein, and an anti-HIV broadly neutralizing antibody (bNAb). In some embodiments, the bNAb binds to epitopes or regions of gp120 are selected from the group consisting of: (i) third variable loop (V3) (e.g., high mannose patch) comprising a N332 oligomannose glycan; (ii) second variable loop (V2) (e.g., Env trimer apex); (iii) CD4 binding site (CD4bs); (iv) gp120/gp41 interface; or (v) silent face of gp120. The foregoing epitopes or regions of gp120 bound by broadly neutralizing antibodies are described, e.g., in McCoy, Retrovirology (2018) 15:70; Sok and Burton, Nat Immunol. 2018 19(11):1179-1188; Possas, et al., Expert Opin Ther Pat. 2018 July; 28(7):551-560; and Stephenson and Barouch, Curr HIV/AIDS Rep (2016) 13:31-37, which are hereby incorporated herein by reference in their entirety for all purposes. Various bNAbs are known in the art and may be combined with the IL-15v, IL-15Rα Sushi domain-IL-15v fusion protein or CD4-targeted IL-15v, as described herein.

In some embodiments, the combination therapy includes an IL-15v, an IL-15Rα Sushi domain-IL-15v fusion protein or CD4-targeted IL-15v, as described herein, and a bNAb that binds to an epitope or region of gp120 in the third variable loop (V3) (e.g., high mannose patch) comprising a N332 oligomannose glycan and competes with or comprises one or more of CDRs and VH and VL regions from an antibody selected from the group consisting of GS-9722 (elipovimab), zinlirvimab (GS-2872), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the combination therapy includes an IL-15v, an IL-15Rα Sushi domain-IL-15v fusion protein or CD4-targeted IL-15v, as described herein, and a bNAb that binds to an epitope or region of gp120 in the CD4 binding site (CD4bs) and competes with or comprises one or more of CDRs and VH and VL regions from an antibody selected from the group consisting of 3BNC117, GS-9723, teropavimab (GS-5423), b12, F105, VRC01, VRC-01-LS, VRC01-23LS, VRC07, VRC07-523, VRC07-523-LS, VRC03, VRC06, VRC06b01 VRC08, VRC0801, VRC-HIVMAB091-00-AB, NIH45-46, 3BNC117, 3BNC60, PGV04 (a.k.a., VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25. In some embodiments, the combination therapy comprises co-administering (i) an IL-15v, an IL-15Rα Sushi domain-IL-15v fusion protein or CD4-targeted IL-15v, as described herein; (ii) an antibody that competes with or comprises VH and VL regions from an antibody selected from the group consisting of 10-1074, 10-1074-J, GS-9722 (elipovimab), zinlirvimab (GS-2872), PGT-121, PGT-121.66, PGT-121.414 and PGT-134; and; (iii) an antibody that competes with or comprises VH and VL regions from an antibody selected from the group consisting of GS-9723, teropavimab (GS-5423), 3BNC117, VRC07 and VRC07-523.

In some embodiments, the combination therapy includes an IL-15v, an IL-15Rα Sushi domain-IL-15v fusion protein or CD4-targeted IL-15v, as described herein, and a bNAb that binds to an epitope or region of gp120 in the second variable loop (V2) (e.g., Env trimer apex) and competes with or comprises one or more of CDRs and VH and VL regions from an antibody selected from the group consisting of PG9, PG16, PGC14, PGG14, PGT-142, PGT-143, PGT-144, PGT-145, CH01, CH59, PGDM1400, CAP256, CAP256-VRC26.08, CAP256-VRC26.09, CAP256-VRC26.25, PCT64-24E and VRC38.01. In some embodiments, the combination therapy includes an IL-15v, an IL-15Rα Sushi domain-IL-15v fusion protein or CD4-targeted IL-15v, as described herein, and a bNAb that binds to an epitope or region of gp120 in the gp120/gp41 interface and competes with or comprises second VH and VL regions from an antibody selected from the group consisting of PGT-151, CAP248-2B, 35022, 8ANC195, ACS202, VRC34 and VRC34.01. In some embodiments, the combination therapy includes an IL-15v, an IL-15Rα Sushi domain-IL-15v fusion protein or CD4-targeted IL-15v, as described herein, and a bNAb that binds to an epitope or region of the gp120 silent face and competes with or comprises second VH and VL regions from an antibody selected from VRC-PG05 and SF12. In some embodiments, the combination therapy includes an IL-15v, an IL-15Rα Sushi domain-IL-15v fusion protein or CD4-targeted IL-15v, as described herein, and a bNAb that binds to an epitope or region of gp41 in the membrane proximal region (MPER) and competes with or comprises second VH and VL regions from an antibody selected from the group consisting of 10E8, 10E8v4, 10E8-5R-100cF, 4E10, DH511.11P, 2F5, 7b2, and LN01. In some embodiments, the combination therapy includes an IL-15v, an IL-15Rα Sushi domain-IL-15v fusion protein or CD4-targeted IL-15v, as described herein, and a bNAb that binds to and epitope or region of the gp41 fusion peptide and competes with or comprises second VH and VL regions from an antibody selected from the group consisting of VRC34 and ACS202.

In certain embodiments, the IL-15v, IL-15Rα Sushi domain-IL-15v fusion protein or CD4-targeted IL-15v, as described herein, is combined or co-administered with a trispecific or trivalent binding molecule that binds to three different HIV epitopes, e.g., as described above, e.g., such as SAR441236, and trispecific or trivalent binding molecules described, e.g., in WO 2014/089152; WO 2016/196740, WO 2017/074878 and WO 2020/210386.

In certain embodiments, the broadly neutralizing antibody may be improved to have enhanced drug-like-properties, reduced immunogenicity, enhanced ADCC, and suitable pharmacokinetic properties (e.g., extended serum half-life). Such antibodies were shown to bind to the HIV envelope glycoprotein expressed on the surface of virion or infected cells, and mediating both direct neutralization of the virus as well as potent NK, Monocyte and PBMC killing of these cells. This property allows the antibodies to treat HIV infections by neutralizing the virus, and also kill and eliminate latently HIV infected cells in infected individuals, potentially leading to a sterilizing cure for HIV.

b. Combination Therapies with a CD4-Fc Fusion Protein

In certain embodiments, the IL-15v, IL-15Rα Sushi domain-IL-15v fusion protein or CD4-targeted IL-15v, as described herein, is combined or co-administered with a fusion protein comprising at least a CD4 extracellular domain D1 (e.g., D1, D1.22, D1D2) and an immunoglobulin constant region, e.g., a CD4-Fc fusion protein. Illustrative CD4-Fc fusion proteins that can be combined or co-administered are described, e.g., in WO 2011/146891, WO 2019/183387, WO 2022/046644 (e.g., amtabafusp alfa (GS-8588)) and WO 2024/094690. In some embodiments, the IL-15v, IL-15Rα Sushi domain-IL-15v fusion protein or CD4-targeted IL-15v, as described herein, is combined or co-administered with amtabafusp alfa (GS-8588).

c. Combination Therapies with One or More Additional Anti-HIV Therapeutic Agents

### HIV Combination Therapy

In some embodiments, the additional therapeutic agent is a latency reversing agent (LRA). Example LRAs include agonists of a toll-like receptor (TLR), e.g., an agonist of one or more of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and TLR10 (NCBI Gene ID: 81793). In some embodiments, the LRA is a TLR7 agonist. In other embodiments, the additional therapeutic agent is a latency reversing agent (LRA), e.g., a lipid nanoparticle (LNP) comprising HIV tat mRNA, a TLR8 agonist. Examples of TLR agonists include but are not limited to vesatolimod. Additional examples include but are not limited to the compounds described in U.S. Pat. No. 8,367,670 and the compounds described in U.S. Patent Application Publication No. 2016/0289229. In one embodiment, the antibody described herein may be combined with TLR7 agonist such as vesatolimod. In another embodiment, the antibody described herein may be combined with TLR8 agonist, e.g., selgantolimod (GS-9688). In one embodiment, the additional therapeutic agent is a TLR modulator. TLR modulators may include modulators of TLR1, TLR2, TLR3, TLR4, TLR5, TLR6, TLR7, TLR8, TLR9, TLR10, TLR11, TLR12, and TLR13. Examples of TLR3 modulators include rintatolimod, poly-ICLC, RIBOXXON®, Apoxxim, RIBOXXIM®, IPH-33, MCT-465, MCT-475, and ND-1.1. Examples of TLR7 modulators include vesatolimod (GS-9620), GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20100143301 (Gilead Sciences), US20110098248 (Gilead Sciences), and US20090047249 (Gilead Sciences). Examples of TLR8 modulators include selgantolimod (GS-9688), motolimod, resiquimod, 3M-051, 3M-052, MCT-465, IMO-4200, VTX-763, VTX-1463, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Examples of TLR9 modulators (e.g., agonists) include BB-001, BB-006, CYT-003, IMO-2055, IMO-2125, IMO-3100, IMO-8400, IR-103, IMO-9200, AST-008 (cavrotolimod), cobitolimod, CMP-001, agatolimod, DIMS-9054, DV-1079, DV-1179, AZD-1419, leftolimod (MGN-1703), litenimod, CYT-003-QbG10, tilsotolimod and PUL-042.

In some embodiments, the additional therapeutic agent is an agonist of DExD/H-box helicase 58 (DDX58; a.k.a., RIG-I, RIGI, RIGI, RLR-1, SGMRT2; NCBI Gene ID: 23586). An illustrative RIG-I agonist is KIN1148, described by Hemann, et al., J Immunol May 1, 2016, 196 (1 Supplement) 76.1. Additional RIG-I agonists are described, e.g., in Elion, et al., Cancer Res. (2018) 78(21):6183-6195; and Liu, et al., J Virol. (2016) 90(20):9406-19. RIG-I agonists are commercially available, e.g., from Invivogen (invivogen.com).

In certain embodiments, such formulations are suitable for once daily, once weekly, once bi-weekly, once monthly, once every two months, or once every three months dosing.

In some embodiments, the additional therapeutic agent may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators (e.g., immunostimulators), immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, compounds that target the HIV capsid, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein inhibitors, RNA polymerase modulators, TAT protein inhibitors, Prolyl endopeptidase (PE) inhibitors, Phospholipase A2 inhibitors, pharmacokinetic enhancers, HIV gene therapy, HIV vaccines, anti-HIV peptides, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are selected from dolutegravir, cabotegravir, islatravir, darunavir, bictegravir, elsulfavirine, rilpivirine, and lenacapavir, and combinations thereof.

### HIV Long Acting Therapies

In some embodiments, the IL-15v, IL-15Rα Sushi domain-IL-15v fusion protein or CD4-targeted IL-15v, as described herein, are combined or co-administered with an HIV long-acting anti-HIV regimen. Examples of as long acting regimens that can be combined or co-adminisered include without limitation cabotegravir, rilpivirine, any integrase LA, VM-1500 LAI, maraviroc (LAI), tenofovir implant, islatravir implant, doravirine, raltegravir, and long acting dolutegravir.

### HIV Combination Drugs

In certain embodiments, the IL-15v, IL-15Rα Sushi domain-IL-15v fusion protein or CD4-targeted IL-15v, as described herein, is combined or co-administered with an HIV combination drug. Examples of combination drugs that can be employed with the IL-15v, IL-15Rα Sushi domain-IL-15v fusion protein or CD4-targeted IL-15v, as described herein, include without limitation ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir and lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; PREZCOBIX® (darunavir and cobicistat); dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, HA-722 (dolutegravir+lamivudine+tenofovir disoproxil fumarate), dolutegravir+lamivudine+tenofovir alafenamide; lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, ACC-008 (ACC-007+lamivudine+tenofovir disoproxil fumarate), elsulfavirine+emtricitabine+tenofovir disoproxil, tenofovir disoproxil fumarate+emtricitabine+rilpivirine hydrochloride, lopinavir, ritonavir, zidovudine, lopinavir+ritonavir+abacavir+lamivudine, and lamivudine; cabotegravir+rilpivirine; 3BNC117+albuvirtide, elsulfavirine (VM-1500), depulfavirine (VM-1500A), lenacapavir+islatravir (oral, injectable), and dual-target HIV-1 reverse transcriptase/nucleocapsid protein 7 inhibitors.

### Other HIV Drugs

Examples of other drugs for treating HIV that can be combined with the IL-15v, IL-15Rα Sushi domain-IL-15v fusion protein or CD4-targeted IL-15v, as described herein, include aspernigrin C, acemannan, alisporivir, BanLec, deferiprone, Gamimune, metenkefalin, naltrexone, Prolastin, REP 9, RPI-MN, VSSP, Hlviral, SB-728-T, 1,5-dicaffeoylquinic acid, rHIV7-shl-TAR-CCR5RZ, AAV-eCD4-Ig gene therapy, MazF gene therapy, BlockAide, bevirimat, ABBV-382, obefazimod (ABX-464), ABX-464, AG-1105, APH-0812, APH0202, bryostatin-1, bryostatin-23, bryostatin analogs, BIT-225, BRII-732, BRII-778, Codivir, CYT-107, CS-TATI-1, fluoro-beta-D-arabinose nucleic acid (FANA)-modified antisense oligonucleotides, FX-101, GILENYA® (fingolimod), griffithsin, HGTV-43, HPH-116, HRS-5685, HivCide-I, hydroxychloroquine, IMB-10035, IMO-3100, IND-02, JL-18008, LADAVRU, MK-1376, MK-2048, MK-4250, ulonivirine (MK-8507), MK-8558, islatravir (MK-8591) (islatravir), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, x1-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, T-1144, VIR-576, nipamovir, Covimro, WP-1122, ZFP-362, MK-8510, LP-98, UVAX-1197, and ABBV-1882.

### HIV Protease Inhibitors

In certain embodiments, the IL-15v, IL-15Rα Sushi domain-IL-15v fusion protein or CD4-targeted IL-15v, as described herein, are combined or co-administered with an HIV protease inhibitor. Examples of HIV protease inhibitors that can be combined with the IL-15v, IL-15Rα Sushi domain-IL-15v fusion protein or CD4-targeted IL-15v, as described herein, include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, elunonavir (GS-1156), TMB-657 (PPL-100), T-169, BL-008, MK-8122, TMB-607, GRL-02031, TLC-ART-101, and TMC-310911. Additional examples of protease inhibitors that can be combined or co-administered are disclosed in U.S. Pat. Nos. 10,294,234, US2020030327 and US2019210978.

### HIV Ribonuclease H Inhibitors

In certain embodiments, the IL-15v, IL-15Rα Sushi domain-IL-15v fusion protein or CD4-targeted IL-15v, as described herein, are combined or co-administered with an HIV ribonuclease H inhibitor. Examples of HIV ribonuclease H inhibitors that can be combined with the IL-15v, IL-15Rα Sushi domain-IL-15v fusion protein or CD4-targeted IL-15v, as described herein, include NSC-727447.

HIV Nef inhibitors

In certain embodiments, the IL-15v, IL-15Rα Sushi domain-IL-15v fusion protein or CD4-targeted IL-15v, as described herein, are combined or co-administered with an HIV Nef inhibitor. Examples of HIV Nef inhibitors that can be combined with the IL-15v, IL-15Rα Sushi domain-IL-15v fusion protein or CD4-targeted IL-15v, as described herein, include FP-1.

### HIV Reverse Transcriptase Inhibitors

In certain embodiments, the IL-15v, IL-15Rα Sushi domain-IL-15v fusion protein or CD4-targeted IL-15v, as described herein, are combined or co-administered with a non-nucleoside or non-nucleotide inhibitor. Examples of HIV non-nucleoside or non-nucleotide inhibitors of reverse transcriptase that can be combined with the IL-15v, IL-15Rα Sushi domain-IL-15v fusion protein or CD4-targeted IL-15v, as described herein, include dapivirine, delavirdine, delavirdine mesylate, doravirine, difluoro-biphenyl-diarylpyrimidines (DAPY), efavirenz, etravirine, GS-5894, lentinan, nevirapine, rilpivirine, ACC-007, ACC-018, AIC-292, F-18, KM-023, PC-1005, M1-TFV, M2-TFV, VM-1500A-LAI, PF-3450074, elsulfavirine (sustained release oral), doravirine+islatravir (fixed dose combination/oral tablet formulation), elsulfavirine (long-acting injectable nanosuspension), and elsulfavirine (VM-1500).

### Non-Nucleoside Reverse Transcriptase Inhibitors (NNRTIs) as Targeted Activators of Cell Kill (TACK)

Balibar, et al., Sci Transl Med (2023) 15 (684):eabn2038 report that select NNRTIs, such as efavirenz (EFV) and rilpivirine (RPV), bind the reverse transcriptase-p66 domain of monomeric Gag-Pol, acting as allosteric modulators to accelerate dimerization, induce premature intracellular HIV-1 protease activation, and elicit cleavage of host proteins, including the caspase recruitment domain-containing protein 8 (CARD8) inflammasome, thereby leading to pyroptotic cell death, and refers to this subset of NNRTIs as targeted activators of cell kill (TACK). In certain embodiments, the IL-15v, IL-15Rα Sushi domain-IL-15v fusion protein or CD4-targeted IL-15v, as described herein, are combined or co-administered with a TACK non-nucleoside or non-nucleotide inhibitor, e.g., efavirenz (EFV), rilpivirine (RPV), pyrimidones pyr01, pyr02 and those described, e.g., in WO 2020/131597; WO 2020/236692; WO 2022/046844; WO 2022/125412; and WO 2023/064196. In certain embodiments, the IL-15v, IL-15Rα Sushi domain-IL-15v fusion protein or CD4-targeted IL-15v, as described herein, are combined or co-administered with a TACK non-nucleoside or non-nucleotide inhibitor and a DPP9 inhibitor, e.g., talabostat (believed to magnify the effect of TACK drugs).

In certain embodiments, the IL-15v, IL-15Rα Sushi domain-IL-15v fusion protein or CD4-targeted IL-15v, as described herein, are combined or co-administered with an HIV nucleoside or nucleotide inhibitor. Examples of HIV nucleoside or nucleotide inhibitors of reverse transcriptase that can be combined with the IL-15v, IL-15Rα Sushi domain-IL-15v fusion protein or CD4-targeted IL-15v, as described herein, include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir amibufenamide fumarate (HS-10234), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, GSK-4023991, MK-8504, islatravir (MK-8591), MK-8583, VM-2500, CL-197 and KP-1461.

Additional examples of HIV nucleoside or nucleotide inhibitors of reverse transcriptase include, but are not limited to those described in US patent No. US2007049754, US2016250215, US2016237062, US2016251347; US2002119443, US2013065856, US2013090473, US2014221356; and WO04096286.

### HIV Integrase Inhibitors

In certain embodiments, the IL-15v, IL-15Rα Sushi domain-IL-15v fusion protein or CD4-targeted IL-15v, as described herein, are combined or co-administered with an HIV integrase inhibitor. Examples of HIV integrase inhibitors that can be combined with the IL-15v, IL-15Rα Sushi domain-IL-15v fusion protein or CD4-targeted IL-15v, as described herein, include elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir, cabotegravir (long-acting injectable), diketo quinolin-4-1 derivatives, GS-1720, GS-6212, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, S-365598, stilbenedisulfonic acid, T-169, STP-0404, VM-3500, XVIR-110, and ACC-017.

In certain embodiments, the IL-15v, IL-15Rα Sushi domain-IL-15v fusion protein or CD4-targeted IL-15v, as described herein, are combined or co-administered with a HIV non-catalytic site, or allosteric, integrase inhibitor (NCINI). Examples of HIV non-catalytic site, or allosteric, integrase inhibitors (NCINI) that can be combined with the IL-15v, IL-15Rα Sushi domain-IL-15v fusion protein or CD4-targeted IL-15v, as described herein, include CX-05045, CX-05168, and CX-14442. Additional examples of HIV capsid inhibitors that can be combined or co-administered include without limitation those described in U.S. Pat. Nos. US2014221356 and US2016016973.

### HIV Viral Infectivity Factor Inhibitor

In certain embodiments, the IL-15v, IL-15Rα Sushi domain-IL-15v fusion protein or CD4-targeted IL-15v, as described herein, are combined or co-administered with an HIV viral infectivity factor inhibitor. Examples of HIV viral infectivity factor inhibitors that can be combined with the IL-15v, IL-15Rα Sushi domain-IL-15v fusion protein or CD4-targeted IL-15v, as described herein, include 2-amino-N-(2-methoxyphenyl)-6-((4-nitrophenyl)thio)benzamide derivatives, and Irino-L.

### HIV Entry (Fusion) Inhibitors

In certain embodiments, the IL-15v, IL-15Rα Sushi domain-IL-15v fusion protein or CD4-targeted IL-15v, as described herein, are combined or co-administered with an HIV entry (fusion) inhibitor. Examples of HIV entry (fusion) inhibitors that can be combined with the IL-15v, IL-15Rα Sushi domain-IL-15v fusion protein or CD4-targeted IL-15v, as described herein, include AAR-501, LBT-5001, cenicriviroc, CCR5 inhibitors, gp41 inhibitors, CD4 attachment inhibitors, gp120 inhibitors, gp160 inhibitors, CXCR4 inhibitors and D-peptide HIV entry inhibitors (e.g., cholesterol-PIE12-trimer (CPT31)).

In certain embodiments, the IL-15v, IL-15Rα Sushi domain-IL-15v fusion protein or CD4-targeted IL-15v, as described herein, are combined or co-administered with a C-C motif chemokine receptor 5 (CCR5; NCBI Gene ID: 1234) inhibitor. Examples of CCR5 inhibitors that can be combined with the IL-15v, IL-15Rα Sushi domain-IL-15v fusion protein or CD4-targeted IL-15v, as described herein, include aplaviroc, MK-7690 (vicriviroc), maraviroc, maraviroc (long-acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc, vMIP (Haimipu), CCR5/CCR2 dual inhibitors, e.g., cenicriviroc, BMS-813160.

In certain embodiments, the IL-15v, IL-15Rα Sushi domain-IL-15v fusion protein or CD4-targeted IL-15v, as described herein, are combined or co-administered with a CXCR4 inhibitor. Examples of CXCR4 inhibitors that can be combined with the IL-15v, IL-15Rα Sushi domain-IL-15v fusion protein or CD4-targeted IL-15v, as described herein, include plerixafor, ALT-1188, N15 peptide, balixafortide, vMIP (Haimipu), BL-8040, LY2510924, burixafor (TG-0054), X4P-002 and X4P-001-IO.

In certain embodiments, the IL-15v, IL-15Rα Sushi domain-IL-15v fusion protein or CD4-targeted IL-15v, as described herein, are combined or co-administered with a gp41 inhibitor. Examples of gp41 inhibitors that can be combined with the IL-15v, IL-15Rα Sushi domain-IL-15v fusion protein or CD4-targeted IL-15v, as described herein, include albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, C13hmAb, lipovirtide, PIE-12 trimer and sifuvirtide.

In certain embodiments, the IL-15v, IL-15Rα Sushi domain-IL-15v fusion protein or CD4-targeted IL-15v, as described herein, are combined or co-administered with a CD4 attachment inhibitor. Examples of CD4 attachment inhibitors that can be combined with the IL-15v, IL-15Rα Sushi domain-IL-15v fusion protein or CD4-targeted IL-15v, as described herein, include ibalizumab and CADA analogs.

In certain embodiments, the IL-15v, IL-15Rα Sushi domain-IL-15v fusion protein or CD4-targeted IL-15v, as described herein, are combined or co-administered with a gp120 inhibitor. Examples of gp120 inhibitors that can be combined with the IL-15v, IL-15Rα Sushi domain-IL-15v fusion protein or CD4-targeted IL-15v, as described herein, include anti-HIV microbicide, Radha-108 (receptol) 3B3-PE38, BMS818251, BanLec, bentonite-based nanomedicine, fostemsavir tromethamine, IQP-0831, VVX-004, and BMS-663068.

In certain embodiments, the IL-15v, IL-15Rα Sushi domain-IL-15v fusion protein or CD4-targeted IL-15v, as described herein, are combined or co-administered with a gp160 inhibitor. Examples of gp160 inhibitors that can be combined with the IL-15v, IL-15Rα Sushi domain-IL-15v fusion protein or CD4-targeted IL-15v, as described herein, include fangchinoline.

### HIV Maturation Inhibitors

In certain embodiments, the IL-15v, IL-15Rα Sushi domain-IL-15v fusion protein or CD4-targeted IL-15v, as described herein, are combined or co-administered with an HIV maturation inhibitor. Examples of HIV maturation inhibitors that can be combined with the IL-15v, IL-15Rα Sushi domain-IL-15v fusion protein or CD4-targeted IL-15v, as described herein, include BMS-955176, fipravirimat mesylate (GSK-3640254), VH-3739937 (GSK-3739937), HRF-10071 and GSK-2838232.

### Capsid Inhibitors

In certain embodiments, the IL-15v, IL-15Rα Sushi domain-IL-15v fusion protein or CD4-targeted IL-15v, as described herein, are combined or co-administered with a capsid inhibitor. Examples of capsid inhibitors that can be combined with the IL-15v, IL-15Rα Sushi domain-IL-15v fusion protein or CD4-targeted IL-15v, as described herein, include capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-4182, GS-CA1, AVI-621, AVI-101, AVI-201, AVI-301, and AVI-CAN1-15 series, PF-3450074, VH-4004280, VH-4011499, HIV-1 capsid inhibitors (HIV-1 infection, Shandong University), and compounds described in Intl. Patent Publ. No. WO2019/087016. Additional examples of capsid inhibitors that can be combined or co-administered include without limitation those described in U.S. Patent Publ. Nos. US2018051005 and US2016108030.

### Cytochrome P450 3 Inhibitors

In certain embodiments, the IL-15v, IL-15Rα Sushi domain-IL-15v fusion protein or CD4-targeted IL-15v, as described herein, are combined or co-administered with a cytochrome P450 3 inhibitor. Examples of Cytochrome P450 3 inhibitors that can be combined or co-administered include without limitation those described in U.S. Pat. No. 7,939,553.

### RNA Polymerase Modulators

In certain embodiments, the IL-15v, IL-15Rα Sushi domain-IL-15v fusion protein or CD4-targeted IL-15v, as described herein, are combined or co-administered with a RNA polymerase modulator (e.g., inhibitor). Examples of RNA polymerase modulators (e.g., inhibitors) include without limitation those described in U.S. Pat. Nos. 10,065,958 and 8,008,264.

### Latency Reversing Agents

In certain embodiments, the IL-15v, IL-15Rα Sushi domain-IL-15v fusion protein or CD4-targeted IL-15v, as described herein, are combined or co-administered with an HIV latency reversing agent (LRA). Latency-reversing agents reactivate latent HIV within CD4 cells, allowing ART and the body's immune system to attack the virus. See, e.g., /clinicalinfo.hiv.gov/en/glossary/latency-reversing-agents; Kula-Pacurar, et al., Semin Immunol. (2021) 51:101478; Bricker, et al., Viruses. (2021) 13(8):1560; and Ait-Ammar, et al., Front Microbiol. (2020) 10:3060. Examples of latency reversing agents that can be combined with the IL-15v, IL-15Rα Sushi domain-IL-15v fusion protein or CD4-targeted IL-15v, as described herein, include toll-like receptor (TLR) agonists (including TLR7 agonists, e.g., vesatolimod (GS-9620) and TLR8 agonists, e.g., selgantolimod (GS-9688)), TLR9 agonists, e.g., lefitolimod (MGN-1703)), a lipid nanoparticle (LNP) comprising a tat mRNA (e.g., as described in WO 2015/162192; WO 2023/218420; WO 2024/178413; Pardons, et al., Nat Commun. (2023) 14(1):8397; Van Gulck, et al., Antimicrob Agents Chemother. (2023) 67(11):e0041723; and Raines, et al., Antimicrob Agents Chemother (2024) 68(7):e0020124), histone deacetylase (HDAC) inhibitors, proteasome inhibitors such as velcade and ixazomib; protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242, QURA 086), Second Mitochondria-derived Activator of Caspase (SMAC) (a.k.a., DIABLO; NCBI Gene ID: 56616) mimetics or inhibitors of cellular inhibitor of apoptosis proteins (cIAP; a.k.a., cIAP1; BIRC2; NCBI Gene ID: 329 or cIAP2; BIRC3; NCBI Gene ID: 330) (including xevinapant (Debio-1143, AT-406), dasminapant (APG-1387), birinapant (TL32711), tolinapant lactate (ASTX-660), ciapavir, AZD5582, BI-891065, BGB-24714, BV6, GDC-0152, GDC-0917, HGS1029, LCL-161 (NVP-LCL161), SM83, UC-112, and those described in U.S. Pat. Nos. 10,870,663; 11,492,361; and 11,873,310; and Intl. Patent Publ. Nos. WO 2005/084317; WO 2008/128121; WO 2009/060292; WO 2011/002684; WO 2013/049350; WO 2013/124701; WO 2014/060770; and WO 2023/230541), inhibitors of phosphatases protein tyrosine phosphatase non-receptor type 1 (PTPN1; NCBI Gene ID: 5770) and/or protein tyrosine phosphatase non-receptor type 2 (PTPN2; NCBI Gene ID: 5771) (such as 3-Hydroxy-1,2,3-benzotriazin-4(3H)-one (HODHBt) (see, e.g., Howard, et al., JCI Insight (2024) e179680), avarol, osunprotafib, trodusquemine, S-nitrosoglutathione, S-nitroso-N-acetylpenicillamine, talaramide Q, aurothiomalate disodium, daiokanzoto, berberine chloride, alpha-tocopherolquinone, IONIS-PTPIBRx (ISIS-404173), ABBV-CLS-579; HDM-2010), PMA, SAHA (suberanilohydroxamic acid, or suberoyl, anilide, and hydroxamic acid, NIZ-985, JQ1, disulfiram, amphotericin B, and ubiquitin inhibitors such as largazole analogs, APH-0812, and GSK-343. Examples of HDAC inhibitors include romidepsin, vorinostat, panobinostat and tucidinostat (a.k.a., chidamide). Examples of PKC activators include indolactam, prostratin, ingenol mebutate, and DAG-lactones. Additional examples of TLR7 agonists that can be combined or co-administered include without limitation described in U.S. Patent Publ. No. US2010143301. Additional examples of TLR8 agonists that can be combined or co-administered include without limitation described in U.S. Patent Publ. No. US2017071944.

### Histone Deacetylase (HDAC) Inhibitors

In various embodiments, the IL-15v, IL-15Rα Sushi domain-IL-15v fusion protein or CD4-targeted IL-15v, as described herein, is combined or co-administered with an inhibitor of a histone deacetylase, e.g., histone deacetylase 1 (HDAC1; NCBI Gene ID: 3065), histone deacetylase 2 (HDAC2; NCBI Gene ID: 3066), histone deacetylase 3 (HDAC3; NCBI Gene ID: 8841), histone deacetylase 4 (HDAC4; NCBI Gene ID: 9759), histone deacetylase 5 (HDAC5; NCBI Gene ID: 10014), histone deacetylase 6 (HDAC6; NCBI Gene ID: 10013), histone deacetylase 7 (HDAC7; NCBI Gene ID: 51564), histone deacetylase 8 (HDAC8; NCBI Gene ID: 55869), histone deacetylase 9 (HDAC9; NCBI Gene ID: 9734), histone deacetylase 11 (HDAC11; NCBI Gene ID: 79885). Examples of HDAC inhibitors that can be combined with the IL-15v, IL-15Rα Sushi domain-IL-15v fusion protein or CD4-targeted IL-15v, as described herein, include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, tucidinostat (a.k.a., chidamide), remetinostat, and entinostat.

### SMAC Mimetics/cIAP Inhibitors

In certain embodiments, the IL-15v, IL-15Rα Sushi domain-IL-15v fusion protein or CD4-targeted IL-15v, as described herein, are combined or co-administered with a Second Mitochondria-derived Activator of Caspase (SMAC) (a.k.a., DIABLO; NCBI Gene ID: 56616) mimetic or an inhibitor of cellular inhibitor of apoptosis proteins (cIAP; a.k.a., cIAP1; BIRC2; NCBI Gene ID: 329 or cIAP2; BIRC3; NCBI Gene ID: 330). Examples of SMAC mimetics or cIAP inhibitors that can be combined or co-administered include without limitation xevinapant (Debio-1143, AT-406), dasminapant (APG-1387), birinapant (TL32711), tolinapant lactate (ASTX-660), ciapavir, AZD5582 (i.e., CAS 1258392-53-8; IUPAC Name: (2S)-1-[(2S)-2-cyclohexyl-2-[(2S)-2-(methylamino) propanoyl]amino]acetyl]-N-[(1S,2R)-2-[6-[(1S,2R)-1-[[(2S)-1-[(2S)-2-cyclohexyl-2-[(2S)-2-(methylamino) propanoyl]amino]acetyl]pyrrolidine-2-carbonyl]amino]-2,3-dihydro-1H-inden-2-yl]oxy]hexa-2,4-diynoxy]-2,3-dihydro-1H-inden-1-yl]pyrrolidine-2-carboxamide); BI-891065, BGB-24714, BV6, GDC-0152, GDC-0917, HGS1029, LCL-161 (NVP-LCL161), and SM83, UC-112, and those described in U.S. Pat. Nos. 10,870,663; 11,492,361; and 11,873,310; and Intl. Patent Publ. Nos. WO 2005/084317; WO 2008/128121; WO 2009/060292; WO 2011/002684; WO 2013/049350; WO 2013/124701; WO 2014/060770; and WO 2023/230541. AZD5582 is described, e.g., in Hennessey, et al., J. Med. Chem. (2013) 56:9897-9919 and the structure is provided below.

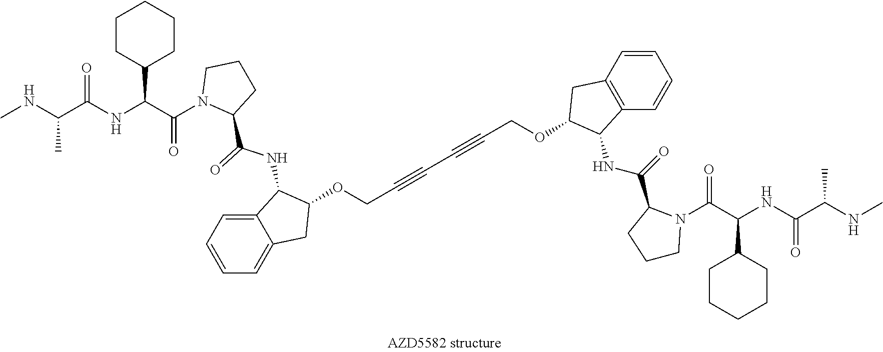

In some embodiments, such combination therapy achieves synergistic effects, e.g., synergistic HIV virion RNA production.

### PTPN1/PTPN2 Inhibitors

In certain embodiments, the IL-15v, IL-15Rα Sushi domain-IL-15v fusion protein or CD4-targeted IL-15v, as described herein, are combined or co-administered with an inhibitor of phosphatases protein tyrosine phosphatase non-receptor type 1 (PTPN1; NCBI Gene ID: 5770) and/or protein tyrosine phosphatase non-receptor type 2 (PTPN2; NCBI Gene ID: 5771). Examples of inhibitors of one or both of PTPN1 and PTPN2 that can be combined or co-administered include without limitation 3-Hydroxy-1,2,3-benzotriazin-4(3H)-one (HODHBt) (see, e.g., Howard, et al., JCI Insight (2024) e179680), avarol, osunprotafib, trodusquemine, S-nitrosoglutathione, S-nitroso-N-acetylpenicillamine, talaramide Q, aurothiomalate disodium, daiokanzoto, berberine chloride, alpha-tocopherolquinone, IONIS-PTP1BRx (ISIS-404173), ABBV-CLS-579; and HDM-2010.

### PTEN Inhibitors

In certain embodiments, the IL-15v, IL-15Rα Sushi domain-IL-15v fusion protein or CD4-targeted IL-15v, as described herein, are combined or co-administered with an inhibitor of phosphatase and tensin homolog (PTEN; NCBI Gene ID: 5728). Examples of PTEN inhibitors that can be combined or co-administered include without limitation bergapten, 5-methoxypsoralen, TAT-PTEN-K13, TAT-r3L4F, GNC-PTEN, ExoPTEN, bpV(phen), bpV(pic), VO-OHpic, SF1670, disulfiram, and those described in WO 2001/07457; WO 2001/90341; WO 2005/097119; and WO 2015/105957. See also, e.g., Spinelli, et al., Adv Biol Regul (2015) 57:102-11; Mac, et al., Methods (2015) 77-78:63-8; and Schmid, et al., FEBS Lett. (2004) 566(1-3):35-8.

### Immune-Based Therapies

In certain embodiments, the IL-15v, IL-15Rα Sushi domain-IL-15v fusion protein or CD4-targeted IL-15v, as described herein, are combined or co-administered with an immune-based therapy. Examples of immune-based therapies include agonists or stimulators of a toll-like receptor (TLR) (e.g., one or more of TLR1, TLR2, TLR3, TLR4, TLR5, TLR6, TLR7, TLR8, TLR9, and TLR10); programmed cell death protein 1 (PD-1) modulators; programmed death-ligand 1 (PD-L1) modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, normferon, peginterferon alfa-2a, peginterferon alfa-2b, RPI-MN, STING modulators, RIG-I modulators, NOD2 modulators, SB-9200, and IR-103.

### Toll-Like Receptor (TLR) Agonists

In various embodiments, the IL-15v, IL-15Rα Sushi domain-IL-15v fusion protein or CD4-targeted IL-15v, as described herein, is combined or co-administered with an agonist of a toll-like receptor (TLR), e.g., an agonist of one or more of TLR1, TLR2, TLR3, TLR4, TLR5, TLR6, TLR7, TLR8, TLR9, and TLR10). Example TLR7 agonists that can be co-administered or combined with the IL-15v, IL-15Rα Sushi domain-IL-15v fusion protein or CD4-targeted IL-15v, as described herein, include without limitation AL-034, DSP-0509, vesatolimod (GS-9620), vesatolimod analogs, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). An TLR7/TLR8 agonist that can be co-administered is NKTR-262, telratolimod and BDB-001. Example TLR8 agonists that can be co-administered or combined with the IL-15v, IL-15Rα Sushi domain-IL-15v fusion protein or CD4-targeted IL-15v, as described herein, include without limitation E-6887, IMO-4200, IMO-8400, IMO-9200, MCT-465, MEDI-9197, motolimod, resiquimod, selgantolimod (GS-9688), VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). Example TLR9 agonists that can be co-administered include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. Examples of TLR4 agonist include G-100, and GSK-1795091. In some embodiments, such combination therapy achieves synergistic effects, e.g., synergistic HIV virion RNA production.

### CDK Inhibitors or Antagonists

In certain embodiments, the IL-15v, IL-15Rα Sushi domain-IL-15v fusion protein or CD4-targeted IL-15v, as described herein, are combined or co-administered with inhibitor of cyclin dependent kinase 1 (CDK1, CDC2; CDC28A; P34CDC2; NCBI Gene ID: 983); cyclin dependent kinase 2 (CDK2, CDKN2; p33 (CDK2); NCBI Gene ID: 1017); cyclin dependent kinase 3 (CDK3; NCBI Gene ID: 1018); cyclin dependent kinase 4 (CDK4, CMM3; PSK-J3; NCBI Gene ID: 1019); cyclin dependent kinase 6 (CDK6, MCPH12; PLSTIRE; NCBI Gene ID: 1021); cyclin dependent kinase 7 (CDK7, CAK; CAK1; HCAK; MO15; STK1; CDKN7; p39MO15; NCBI Gene ID: 1022); cyclin dependent kinase 9 (CDK9, TAK; C-2k; CTK1; CDC2L4; PITALRE; NCBI Gene ID: 1025). Inhibitors of one or more of CDK 1, 2, 3, 4, 6, 7 and 9 that can be combined or co-administered include without limitation abemaciclib, alvocidib (HMR-1275, flavopiridol), AT-7519, dinaciclib, ibrance, FLX-925, LEE001, palbociclib, ribociclib, rigosertib, selinexor, UCN-01, SY1365, CT-7001, SY-1365, G1T38, milciclib, trilaciclib, PF-06873600, AZD4573, and TG-02. In some embodiments, the CDK4/CDK6/CDK9 inhibitor or antagonist is selected from the group consisting of VS2-370.

### Stimulator of Interferon Genes (STING) Agonists

In some embodiments, the IL-15v, IL-15Rα Sushi domain-IL-15v fusion protein or CD4-targeted IL-15v, as described herein, are combined or co-administered with an agonist of stimulator of interferon response cGAMP interactor 1 (STING1; NCBI Gene ID: 340061). In some embodiments, the STING receptor agonist or activator is selected from the group consisting of ADU-S100 (MIW-815), SB-11285, MK-1454, SR-8291, AdVCA0848, GSK-532, SYN-STING, MSA-1, SR-8291, STING agonist (latent HIV), GSK3745417, 5,6-dimethylxanthenone-4-acetic acid (DMXAA), cyclic-GAMP (cGAMP) and cyclic-di-AMP.

### RIG-I Agonists

In certain embodiments, the IL-15v, IL-15Rα Sushi domain-IL-15v fusion protein or CD4-targeted IL-15v, as described herein, are combined or co-administered with an agonist of DExD/H-box helicase 58 (DDX58; a.k.a., RIG-I, RIGI, RIGI, RLR-1, SGMRT2; NCBI Gene ID: 23586). In some embodiments, the agents described herein are combined or co-administered with a RIG-I agonist such as acitretin or RGT-100, or NOD2 modulator, such as SB-9200 (a.k.a., GS 9992; inarigivir soproxil), and IR-103. An illustrative RIG-I agonist is KIN1148, described by Hemann, et al., J Immunol May 1, 2016, 196 (1 Supplement) 76.1. Additional RIG-I agonists are described, e.g., in WO 2017/011622; Elion, et al., Cancer Res. (2018) 78(21):6183-6195; and Liu, et al., J Virol. (2016) 90(20):9406-19. RIG-I agonists are commercially available, e.g., from Invivogen (invivogen.com).

### NOD2 Agonists or Activators

In certain embodiments, the IL-15v, IL-15Rα Sushi domain-IL-15v fusion protein or CD4-targeted IL-15v, as described herein, are combined or co-administered with a nucleotide binding oligomerization domain containing 2 (NOD2; a.k.a., ACUG, BLAU, BLAUS, CARD15, CD, CLR16.3, IBD1, NLRC2, NOD2B, PSORAS1, YAOS; NCBI Gene ID: 64127) agonist or activator. Illustrative NOD2 agonists or activators that can be combined or co-administered include without limitation romurtide (a.k.a., muroctasin), muramyl dipeptide (MDP), muramyl tripeptide, muramyl tetrapeptide, murabutide (Liu, et al., J Cell Physiol. (2019) 234(11):21294-21306), polymuramil (Lesnova, et al., Vopr Virusol. (2023) 68(4):315-326), mifamurtide, M-TriDaP (N-acetyl-muramyl-L-Ala-y-D-Glu-meso-diaminopimelic acid), N-glycolyl muramyldipeptide, M-TriLYS (MurNAc-Ala-D-isoGln-Lys), MDP (D-Glu.sup.2)-OCH₃, Glucosaminyl muramyldipeptide, decoy-20, inarigivir/tenofovir disoproxil fumarate, inarigivir soproxil, SG-101, SG-29, CL429 (Cheng, et al., J Cell Mol Med. (2021) 25(8):3785-3792), norAbu-GMDP-Lys(L18), norAbu-MDP-Lys-B30, and those described, e.g., in WO 2011/020094; WO 2012/021647; WO 2016/172615; WO 2017/011622; WO 2022/084417; WO 2023/161962; WO 2024/102639; and Gutjahr, et al., EBioMedicine. (2020) 58:102922. In some embodiments, such combination therapy achieves synergistic effects, e.g., synergistic HIV virion RNA production.

### DDX3 Inhibitors

In certain embodiments, the IL-15v, IL-15Rα Sushi domain-IL-15v fusion protein or CD4-targeted IL-15v, as described herein, are combined or co-administered with an inhibitor of DEAD-box helicase 3 X-linked (DDX3X; a.k.a., DDX3, CAP-Rf, DBX, DDX14, DDX3, HLP2, MRX102, MRXSSB; NCBI Gene ID: 1654). Illustrative DDX3 inhibitors that can be combined or co-administered include without limitation CAS Reg. No. 1985601-65-7, diimidazo[4,5-d:4′,5′-f]-[1,3]diazepine (RK-33), NZ-51, and those described, e.g., in WO 2008/023077 (K7 polypeptide); WO 2011/039735; WO 2015/200779; WO 2016/128541; WO 2017/162834; WO 2024/081018; Kukhanova, et al., Molecules (2020) 25(4):1015; Bol, et al., Mol Cancer (2015) 14:188; Abdelkrim, et al., Methods Mol Biol (2021) 2209:35-52 and Rao, et al., Nat Commun (2021) 12(1):2475.

LAG-3 and TIM-3 inhibitors

In certain embodiments, the IL-15v, IL-15Rα Sushi domain-IL-15v fusion protein or CD4-targeted IL-15v, as described herein, are combined or co-administered with an anti-TIM-3 (a.k.a., hepatitis A virus cellular receptor 2 antibody (HAVCR2; NCBI Gene ID: 84868), such as TSR-022, LY-3321367, MBG-453, INCAGN-2390. In some embodiments, the IL-15v, IL-15Rα Sushi domain-IL-15v fusion protein or CD4-targeted IL-15v, as described herein, are combined or co-administered with an anti-lymphocyte activating 3 (LAG3, a.k.a., CD223; NCBI Gene ID: 3902) antibody, such as relatlimab (ONO-4482), LAG-525, MK-4280, REGN-3767, INCAGN2385.

### Cytokine Receptor Agonists

In various embodiments, the IL-15v, IL-15Rα Sushi domain-IL-15v fusion protein or CD4-targeted IL-15v, as described herein, are combined or co-administered with one or more cytokine receptor or chemokine receptor agonists. Illustrative cytokine or chemokine receptor agonists that can be co-administered include without limitation IL-12, IL-18, gamma chain (IL2RG; CD132; NCBI Gene ID: 3561)-dependent cytokines (e.g., IL-2, IL-7, IL-9 and IL-21) and fms related tyrosine kinase 3 (FLT3) ligand (FLT3LG).

Examples of IL-2 receptor agonists that can be combined or co-administered include proleukin (aldesleukin, IL-2); BC-IL (Cel-Sci), pegylated IL-2 (e.g., NKTR-214 (bempegaldesleukin)); modified variants of IL-2 (e.g., THOR-707), AIC-284, ALKS-4230, CUI-101 and Neo-2/15.

Examples of IL-12 receptor agonists that can be combined or co-administered include IL-12A (NCBI Gene ID: 3592)+IL-12B (NCBI Gene ID: 3593) mRNAs, such as MEDI1191; IL-12 gene stimulators, such as EGEN-001, tavokinogene telseplasmid; dendritic cells transduced to express IL-12, such as DC-RTS-IL-12; autologous T-Cells genetically engineered to secrete IL-12 and to target the mucin 16, cell surface associated (MUC16, a.k.a., CA125; NCBI Gene ID: 94025), such as JCAR-020; and mRNA encoding the cytokines interleukin-12 single chain (IL-12sc), interferon alpha (IFNα) and granulocyte-macrophage colony-stimulating factor (GM-CSF), such as SAR441000 (BNT131).

An illustrative IL-7 receptor agonist that can be co-administered includes CYT-107.

### PD-1 Targeted Cytokine Receptor Agonists

In various embodiments, the IL-15v, IL-15Rα Sushi domain-IL-15v fusion protein or CD4-targeted IL-15v, as described herein, are combined or co-administered with one or more PD-1-targeted cytokine receptor agonists. In some embodiments, the cytokine is a gamma chain-dependent cytokine e.g., IL-2, IL-7 and IL-21. Illustrative PD-1 targeted IL-2 receptor agonists that can be combined or co-administered include without limitation eciskafusp alfa, IBI-363, ANV-600, ASKG-812, AWT-020, KY-0118, MB-4, MBS-309, MDNA-223, PTX-912, XTX-501, and those described in e.g., WO 2018/176505; WO 2018/184964; WO 2022/192898; WO 2022/212614; WO 2022/266598; WO 2023/010032; WO 2023/151661; WO 2023/281479; WO 2024/026449; WO 2024/133823; WO 2024/150174 and WO 2024/163545. Illustrative PD-1 targeted IL-7 receptor agonists that can be combined or co-administered include without limitation BICKI-IL-7, TJ-L117, and those described in e.g., WO 2020/127377, WO 2023/281484, and WO 2024/150158. Illustrative PD-1 targeted IL-21 receptor agonists that can be combined or co-administered include without limitation latikafusp, and those described in e.g., WO 2019/028316, WO 2021/092719, and WO 2023/048516.

Illustrative fms related tyrosine kinase 3 (FLT3) ligand (FLT3LG) agonists that can be combined or co-administered include GS-3583 (described in WO 2020/263830, among others of use) and CDX-301.

### Interferon Receptor Ligands

In various embodiments, the IL-15v, IL-15Rα Sushi domain-IL-15v fusion protein or CD4-targeted IL-15v, as described herein, are combined or co-administered with one or more interferon receptor (e.g., interferon alpha and beta receptor subunit 1 (IFNAR1; NCBI Gene ID: 3454); interferon alpha and beta receptor subunit 2 (IFNAR2; NCBI Gene ID: 3455); interferon gamma receptor 1 (IFNGR1; NCBI Gene ID: 3459); interferon gamma receptor 2 (IFNGR2; NCBI Gene ID: 3460) ligands, which can be one or more of recombinant, PEGylated, fusion proteins and conjugates. Examples of interferon receptor ligands that can be combined or co-administered include an interferon alpha-1b, an interferon alpha-2a, an interferon alpha-2b, an interferon beta-la, and an interferon gamma. Illustrative interferon alpha-1b that can be combined or co-administered include without limitation, recombinant human interferon alpha-1b, interferon alpha 1b, PEGylated interferon alpha-1b, interferon alpha 1b (HAPGEN®). Illustrative interferon alpha-2a that can be combined or co-administered include without limitation, recombinant human interferon alpha-2a, interferon alfa 2a, PEG-IFN-alpha, pegylated interferon alpha-2a (PEGASYS®), YPEG-interferon alfa-2a (YPEG-rhIFNalpha-2a), interferon alpha-2a biosimilar (Biogenomics), rHSA-IFN alpha-2a (recombinant human serum albumin interferon alpha 2a fusion protein), interferon alfa-2a follow-on biologic (Biosidus) (Inmutag, Inter 2A). Illustrative interferon alpha-2b that can be combined or co-administered include without limitation, recombinant human interferon alpha-2b, alpha-2b (INTRON A®), interferon alfa-2b (from numerous sources, including, e.g., Amega, Axxo, IFN, Laboratorios Bioprofarma, Virchow, Zydus-Cadila, BioGeneric Pharma, Changchun Institute of Biological Products), ropeginterferon alfa-2b (AOP-2014, P-1101, PEG IFN alpha-2b), peginterferon alfa-2b (Amega), Ypeginterferon alfa-2b (YPEG-rhIFNalpha-2b), peginterferon alfa-2b (PEG-INTRON®), rHSA-IFN alpha 2b (recombinant human serum albumin interferon alpha 2b fusion protein), veltuzumab-IFN alpha 2b conjugate, interferon alfa-2b follow-on biologic (Biosidus-Bioferon, Citopheron, Ganapar, Beijing Kawin Technology-Kaferon). Additional illustrative interferon alpha and beta receptor ligands that can be combined or co-administered include without limitation Veldona, Infradure, Roferon-A, Algeron, Alfarona, Ingaron (interferon gamma), rSIFN-co (recombinant super compound interferon), MOR-22, Bioferon, Novaferon, Inmutag (Inferon), MULTIFERON® (Alfanative, Viragen), interferon alfa-n1 (HUMOFERON®, SM-10500, Sumiferon), Shaferon, Alfaferone, interferon-alpha 2 (CJ), Laferonum, VIPEG, BLAUFERON-A, BLAUFERON-B, Dynavax (SD-101), Intermax Alpha, Realdiron, Lanstion, Pegaferon, PDferon-B PDferon-B, Pegnano, Feronsure, PegiHep, Optipeg A, Realfa 2B, Reliferon, Reaferon-EC, Roferon-A (Canferon, Ro-25-3036), Proquiferon, Uniferon, Urifron, Anterferon, Shanferon, Layfferon, Shang Sheng Lei Tai, INTEFEN, SINOGEN, Fukangtai, Pegstat, SFR-9216, Interapo (Interapa), GEPON®, NORMFERON™. Illustrative interferon beta-la that can be combined or co-administered include without limitation, interferon beta-1a (AVONEX®). Illustrative interferon gamma receptor ligands that can be combined or co-administered include without limitation, interferon gamma (OH-6000, Ogamma 100) and RPI-MN (modified cobratoxin).

### Immune Checkpoint Receptor Protein Modulators

In various embodiments, the IL-15v, IL-15Rα Sushi domain-IL-15v fusion protein or CD4-targeted IL-15v, as described herein, are combined or co-administered with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu, et al., J Exp Clin Cancer Res. (2018) 37:110). In various embodiments, the immune checkpoint proteins or receptors regulate NK cell responses (e.g., reviewed in Davis, et al., Semin Immunol. (2017) 31:64-75 and Chiossone, et al., Nat Rev Immunol. (2018) 18(11):671-688).

Examples of immune checkpoint proteins or receptors that can be combined with the IL-15v, IL-15Rα Sushi domain-IL-15v fusion protein or CD4-targeted IL-15v, as described herein, include without limitation CD27 (NCBI Gene ID: 939); CD70 (NCBI Gene ID: 970); CD40 (NCBI Gene ID: 958); CD40LG (NCBI Gene ID: 959); CD47 (NCBI Gene ID: 961); CD48 (SLAMF2; NCBI Gene ID: 962); transmembrane and immunoglobulin domain containing 2 (TMIGD2, CD28H; NCBI Gene ID: 126259); CD84 (LY9B, SLAMF5; NCBI Gene ID: 8832); CD96 (NCBI Gene ID: 10225); CD160 (NCBI Gene ID: 11126); MS4A1 (CD20; NCBI Gene ID: 931); CD244 (SLAMF4; NCBI Gene ID: 51744); CD276 (B7H3; NCBI Gene ID: 80381); V-set domain containing T cell activation inhibitor 1 (VTCN1, B7H4; NCBI Gene ID: 79679); V-set immunoregulatory receptor (VSIR, B7H5, VISTA; NCBI Gene ID: 64115); immunoglobulin superfamily member 11 (IGSF11, VSIG3; NCBI Gene ID: 152404); natural killer cell cytotoxicity receptor 3 ligand 1 (NCR3LG1, B7H6; NCBI Gene ID: 374383); HERV-H LTR-associating 2 (HHLA2, B7H7; NCBI Gene ID: 11148); inducible T cell co-stimulator (ICOS, CD278; NCBI Gene ID: 29851); inducible T cell co-stimulator ligand (ICOSLG, B7H2; NCBI Gene ID: 23308); TNF receptor superfamily member 4 (TNFRSF4, OX40; NCBI Gene ID: 7293); TNF superfamily member 4 (TNFSF4, OX40L; NCBI Gene ID: 7292); TNFRSF8 (CD30; NCBI Gene ID: 943); TNFSF8 (CD30L; NCBI Gene ID: 944); TNFRSF10A (CD261, DR4, TRAILR1; NCBI Gene ID: 8797); TNFRSF9 (CD137; NCBI Gene ID: 3604); TNFSF9 (CD137L; NCBI Gene ID: 8744); TNFRSF10B (CD262, DR5, TRAILR2; NCBI Gene ID: 8795); TNFRSF10 (TRAIL; NCBI Gene ID: 8743); TNFRSF14 (HVEM, CD270; NCBI Gene ID: 8764); TNFSF14 (HVEML; NCBI Gene ID: 8740); CD272 (B and T lymphocyte associated (BTLA); NCBI Gene ID: 151888); TNFRSF17 (BCMA, CD269; NCBI Gene ID: 608); TNFSF13B (BAFF; NCBI Gene ID: 10673); TNFRSF18 (GITR; NCBI Gene ID: 8784); TNFSF18 (GITRL; NCBI Gene ID: 8995); MHC class I polypeptide-related sequence A (MICA; NCBI Gene ID: 100507436); MHC class I polypeptide-related sequence B (MICB; NCBI Gene ID: 4277); CD274 (CD274, PDL1, PD-L1; NCBI Gene ID: 29126); programmed cell death 1 (PDCD1, PD1, PD-1; NCBI Gene ID: 5133); cytotoxic T-lymphocyte associated protein 4 (CTLA4, CD152; NCBI Gene ID: 1493); CD80 (B7-1; NCBI Gene ID: 941); CD28 (NCBI Gene ID: 940); nectin cell adhesion molecule 2 (NECTIN2, CD112; NCBI Gene ID: 5819); CD226 (DNAM-1; NCBI Gene ID: 10666); Poliovirus receptor (PVR) cell adhesion molecule (PVR, CD155; NCBI Gene ID: 5817); PVR related immunoglobulin domain containing (PVRIG, CD112R; NCBI Gene ID: 79037); T cell immunoreceptor with Ig and ITIM domains (TIGIT; NCBI Gene ID: 201633); T cell immunoglobulin and mucin domain containing 4 (TIMD4; TIM4; NCBI Gene ID: 91937); hepatitis A virus cellular receptor 2 (HAVCR2, TIMD3, TIM3; TIM-3; NCBI Gene ID: 84868); galectin 9 (LGALS9; NCBI Gene ID: 3965); lymphocyte activating 3 (LAG3, LAG-3; CD223; NCBI Gene ID: 3902); signaling lymphocytic activation molecule family member 1 (SLAMF1, SLAM, CD150; NCBI Gene ID: 6504); lymphocyte antigen 9 (LY9, CD229, sialic acid binding Ig like lectin 7 (SIGLEC7; p75; QA79; AIRM1; CD328; CDw328; D-siglec; SIGLEC-7; SIGLECP2; SIGLEC19P; p75/AIRM1; NCBI Gene ID: 27036); sialic acid binding Ig like lectin 9 (SIGLEC9; CD329; CDw329; FOAP-9; siglec-9; OBBP-LIKE; NCBI Gene ID: 27180); SLAMF3; NCBI Gene ID: 4063); SLAM family member 6 (SLAMF6, CD352; NCBI Gene ID: 114836); SLAM family member 7 (SLAMF7, CD319; NCBI Gene ID: 57823); UL16 binding protein 1 (ULBP1; NCBI Gene ID: 80329); UL16 binding protein 2 (ULBP2; NCBI Gene ID: 80328); UL16 binding protein 3 (ULBP3; NCBI Gene ID: 79465); retinoic acid early transcript 1E (RAETIE; ULBP4; NCBI Gene ID: 135250); retinoic acid early transcript 1G (RAETIG; ULBP5; NCBI Gene ID: 353091); retinoic acid early transcript 1L (RAET1L; ULBP6; NCBI Gene ID: 154064); killer cell lectin like receptor C1 (KLRC1, NKG2A, CD159A; NCBI Gene ID: 3821); killer cell lectin like receptor K1 (KLRK1, NKG2D, CD314; NCBI Gene ID: 22914); killer cell lectin like receptor C2 (KLRC2, CD159c, NKG2C; NCBI Gene ID: 3822); killer cell lectin like receptor C3 (KLRC3, NKG2E; NCBI Gene ID: 3823); killer cell lectin like receptor C4 (KLRC4, NKG2F; NCBI Gene ID: 8302); killer cell lectin like receptor D1 (KLRD1; NCBI Gene ID: 3824); killer cell lectin like receptor G1 (KLRG1; 2F1; MAFA; MAFA-L; CLEC15A; MAFA-2F1; MAFA-LIKE; NCBI Gene ID: 10219); killer cell immunoglobulin like receptor, two Ig domains and long cytoplasmic tail 1 (KIR2DL1; NCBI Gene ID: 3802); killer cell immunoglobulin like receptor, two Ig domains and long cytoplasmic tail 2 (KIR2DL2; NCBI Gene ID: 3803); killer cell immunoglobulin like receptor, two Ig domains and long cytoplasmic tail 3 (KIR2DL3; NCBI Gene ID: 3804); killer cell immunoglobulin like receptor, three Ig domains and long cytoplasmic tail 1 (KIR3DL1, KIR, CD158E1; NCBI Gene ID: 3811) (e.g., Lirilumab (IPH2102/BMS-986015), IPH-4102) and Hematopoietic Progenitor Kinase 1 (HPK1, MAP4K1).

In various embodiments, the IL-15v, IL-15Rα Sushi domain-IL-15v fusion protein or CD4-targeted IL-15v, as described herein, are combined or co-administered with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the IL-15v, IL-15Rα Sushi domain-IL-15v fusion protein or CD4-targeted IL-15v, as described herein, is combined or co-administered with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu, et al., J Exp Clin Cancer Res. (2018) 37:110.

In various embodiments, the IL-15v, IL-15Rα Sushi domain-IL-15v fusion protein or CD4-targeted IL-15v, as described herein, are combined or co-administered with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the IL-15v, IL-15Rα Sushi domain-IL-15v fusion protein or CD4-targeted IL-15v, as described herein, is combined or co-administered with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis, et al., Semin Immunol. (2017) 31:64-75; Fang, et al., Semin Immunol. (2017) 31:37-54; and Chiossone, et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, envafolimab (ASC-22, KN-035), PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181 (budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Inhibitors of T Cell Immunoreceptor with Ig and ITIM Domains (TIGIT)

In various embodiments, the immunogenic polypeptides, polynucleotides encoding such polypeptides, vectors, LNPs and immunogenic compositions comprising such polypeptides or polynucleotides, as described herein, are combined or co-administered with one or more inhibitors of T cell immunoreceptor with Ig and ITIM domains (TIGIT) (NCBI Gene ID: 201633). Example anti-TIGIT antibodies, that can be combined or co-administered include etigilimab, BMS-986207, tiragolumab (a.k.a., MTIG-7192A; RG-6058; RO 7092284), vibostolimab (MK-7684), ociperlimab (BGB-A1217), domvanalimab (AB154), AGEN1307, AGEN1327, AGEN1777, COM-902, IBI-939, AB154, SGN-TGT, MG1131 and EOS884448 (EOS-448).

### TNF Receptor Superfamily (TNFRSF) Member Agonists or Activators

In various embodiments, the IL-15v, IL-15Rα Sushi domain-IL-15v fusion protein or CD4-targeted IL-15v, as described herein, are combined or co-administered with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Example anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Example anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Example anti-TNFRSF9 (4-1BB, CD137) antibodies that can be co-administered include without limitation urelumab, utomilumab (PF-05082566), AGEN2373 and ADG-106.

Example anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi- and Tri-Specific T-Cell Engagers

In various embodiments, the IL-15v, IL-15Rα Sushi domain-IL-15v fusion protein or CD4-targeted IL-15v, as described herein, are combined or co-administered with a bi-specific T-cell engager (e.g., BiTE) or a tri-specific T-cell engager or a bi-specific antibody (e.g., having an Fc) against a T-cell activating receptor, e.g., CD3. Illustrative multispecific T-cell engaging (e.g., CD3 binding) molecules useful for treating or preventing HIV co-target CD3 and an HIV antigen, e.g., HIV gp120, HIV gp41, HLA-A*02 restricted peptide SLYNTVATL (SEQ ID NO: 1130) derived from the HIV Gag gene product, p17. In various embodiments, the binding domain that binds to the HIV antigen has one or more CD4 domains (e.g., D1, D1.22, D1D2) or has the VH/VL of a broadly neutralizing antibody. Illustrative multispecific T-cell engaging (e.g., CD3 binding) molecules useful for treating or preventing HIV and which can be combined or co-administered with the IL-15v, IL-15Rα Sushi domain-IL-15v fusion protein or CD4-targeted IL-15v, as described herein, include without limitation amtabafusp alfa (GS-8588), MGD014, MGD020, IMC-M113V, and those described, e.g., in WO 1993/08829; WO 1994/04690; WO 2013/163427; WO 2016/054101; WO 2017/163064; WO 2018/053328; WO 2018/183139; WO 2018/237148; WO 2019/222104; WO 2022/046644 (e.g., amtabafusp alfa (GS-8588)); WO 2023/156663.

### Bi- and Tri-Specific Natural Killer (NK)-Cell Engagers

In various embodiments, the IL-15v, IL-15Rα Sushi domain-IL-15v fusion protein or CD4-targeted IL-15v, as described herein, are combined or co-administered with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (4-1BB). Illustrative anti-CD16 bi-specific antibodies, BiKEs or TriKEs that can be co-administered include AFM26 (BCMA/CD16A) and AFM-13 (CD16/CD30). As appropriate, the anti-CD16 binding bi-specific molecules may or may not have an Fc. BiKEs and TriKEs are described, e.g., in Felices, et al., Methods Mol Biol. (2016) 1441:333-346; Fang, et al., Semin Immunol. (2017) 31:37-54. Examples of a trispecific NK cell engager (TRIKE) include OXS-3550, and HIV-TriKE.

Indoleamine-pyrrole-2,3-dioxygenase (IDO1) inhibitors

In various embodiments, the IL-15v, IL-15Rα Sushi domain-IL-15v fusion protein or CD4-targeted IL-15v, as described herein, are combined or co-administered with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Phosphatidylinositol 3-Kinase (PI3K) Inhibitors

In certain embodiments, the IL-15v, IL-15Rα Sushi domain-IL-15v fusion protein or CD4-targeted IL-15v, as described herein, are combined or co-administered with an inhibitor of at least one of a phosphatidylinositol-4,5-bisphosphate 3-kinase catalytic subunit, e.g., phosphatidylinositol-4,5-bisphosphate 3-kinase catalytic subunit alpha (PIK3CA, CLAPO, CLOVE, CWS5, MCAP, MCM, MCMTC, PI3K, PI3K-alpha, p110-alpha; NCBI Gene ID: 5290); phosphatidylinositol-4,5-bisphosphate 3-kinase catalytic subunit beta (PIK3CB, P110BETA, PI3K, PI3KBETA, PIK3C1; NCBI Gene ID: 5291); phosphatidylinositol-4,5-bisphosphate 3-kinase catalytic subunit gamma (PIK3CG, PI3CG, PI3K, PI3Kgamma, PIK3, p110gamma, p120-PI3K; Gene ID: 5494); and phosphatidylinositol-4,5-bisphosphate 3-kinase catalytic subunit delta (PIK3CD, APDS, IMD14, P110DELTA, PI3K, p110D, NCBI Gene ID: 5293). In some embodiments, the PI3K inhibitor is a pan-PI3K inhibitor. Examples of PI3K inhibitors that can be combined or co-administered include without limitation, ACP-319, AEZA-129, AMG-319, AS252424, AZD8186, BAY 1082439, BEZ235, bimiralisib (PQR309), buparlisib (BKM120), BYL719 (alpelisib), carboxyamidotriazole orotate (CTO), CH5132799, CLR-457, CLR-1401, copanlisib (BAY 80-6946), DS-7423, duvelisib (IPI-145), fimepinostat (CUDC-907), gedatolisib (PF-05212384), GDC-0032, GDC-0084 (RG7666), GDC-0077, pictilisib (GDC-0941), GDC-0980, GSK2636771, GSK2269577, idelalisib (Zydelig®), INCB040093, INCB50465, IPI-443, IPI-549, KAR4141, LY294002, LY3023414, NERLYNX® (neratinib), nemiralisib (GSK2269557), omipalisib (GSK2126458, GSK458), OXY111A, panulisib (P7170, AK151761), PA799, perifosine (KRX-0401), Pilaralisib (SAR245408; XL147), puquitinib mesylate (XC-302), SAR260301, seletalisib (UCB-5857), serabelisib (INK-1117,MLN-1117,TAK-117), SF1126, sonolisib (PX-866), RG7604, rigosertib sodium (ON-01910 sodium), RP5090, tenalisib (RP6530), RV-1729, SRX3177, taselisib, TG100115, umbralisib (TGR-1202), TGX221, voxtalisib (SAR245409), VS-5584, WX-037, X-339, X-414, XL499, XL756, wortmannin, ZSTK474, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

### Alpha-4/Beta-7 Antagonists

In certain embodiments, the IL-15v, IL-15Rα Sushi domain-IL-15v fusion protein or CD4-targeted IL-15v, as described herein, are combined or co-administered with an alpha-4/beta-7 antagonist. Examples of Integrin alpha-4/beta-7 antagonists include PTG-100, TRK-170, abrilumab, etrolizumab, carotegrast methyl, and vedolizumab.

### HPK1 Inhibitors

In certain embodiments, the IL-15v, IL-15Rα Sushi domain-IL-15v fusion protein or CD4-targeted IL-15v, as described herein, are combined or co-administered with an inhibitor of mitogen-activated protein kinase kinase kinase kinase 1 (MAP4K1; NCBI Gene ID: 11184; a.k.a., Hematopoietic Progenitor Kinase 1 (HPK1)). Examples of HPK1 inhibitors include, but are not limited to, ZYF-0272, and ZYF-0057.

### BCL2 Inhibitors

In certain embodiments, the IL-15v, IL-15Rα Sushi domain-IL-15v fusion protein or CD4-targeted IL-15v, as described herein, are combined or co-administered with a BCL2 apoptosis regulator (BCL2; a.k.a., Bcl-2, PPP1R50; NCBI Gene ID: 596) inhibitor. Illustrative BCL2 inhibitors that can be combined or co-administered include without limitation venetoclax, sonrotoclax, lisaftoclax, navitoclax, foselutoclax, pelcitoclax, surzetoclax, obatoclax mesylate, oblimersen sodium, TGRX-814, ZN-d5, S-65487 (VOB-560), ICP-248, TQB-3909, FCN-338, UBX-1967, AZD-0466, ZE50-0134, BGB-21447, LP-118, LP-108, and those described in, e.g., WO 2006/069186; WO 2012/097133; WO 2017/153484; WO 2018/009444; WO 2018/041248; WO 2019/001383; WO 2019/112381; WO 2019/185025; WO 2020/088442; WO 2020/024916; WO 2020/238785; WO 2020/229429; WO 2021/007303; WO 2021/110097; WO 2021/175321; WO 2021/208963; WO 2021/219065; WO 2022/012481; WO 2022/028353; WO 2022/148439, WO 2023/030363; and WO 2023/104043.

### Autophagy Inhibitors

In certain embodiments, the IL-15v, IL-15Rα Sushi domain-IL-15v fusion protein or CD4-targeted IL-15v, as described herein, are combined or co-administered with a autophagy inhibitor. Illustrative autophagy inhibitors that can be combined or co-administered include without limitation plitidepsin, ezurpimtrostat hydrochloride, bulaquine/chloroquine phosphate, hydroxychloroquine hydrosulfate, chloroquine phosphate, carnitine orotate complex, proguanil hydrochloride/chloroquine phosphate, biphenyldimethyldicarboxylate, sirolimus/hydroxychloroquine, hydroxychloroquine niosomes, Tat-Atg5K130R, and those described in, e.g., WO 2011/011522; WO 2011/017809; WO 2011/106684; WO 2011/112623; WO 2018/003829; WO 2019/071875; and WO 2020/204441. Employing a method of a selective elimination of host cells harboring replication-competent HIV (SECH) approach with the combination of latency reversal, inhibition of autophagy and induction of apoptosis, Li, et al., Nat Commun. (2020) 11(1):4051 showed that it is feasible to clear host cells harboring replication-competent HIV-1 in humanized mice in vivo, as well as in blood samples from HIV-1-infected patients in vitro.

### Pharmacokinetic Enhancers

In certain embodiments, the IL-15v, IL-15Rα Sushi domain-IL-15v fusion protein or CD4-targeted IL-15v, as described herein, are combined or co-administered with a pharmacokinetic enhancer. Examples of pharmacokinetic enhancers include cobicistat and ritonavir.

### Additional Therapeutic Agents

Examples of additional therapeutic agents include the compounds disclosed in WO 2004/096286 (Gilead Sciences); WO 2006/015261 (Gilead Sciences); WO 2006/110157 (Gilead Sciences); WO 2012/003497 (Gilead Sciences); WO 2012/003498 (Gilead Sciences); WO 2012/145728 (Gilead Sciences); WO 2013/006738 (Gilead Sciences); WO 2013/159064 (Gilead Sciences); WO 2014/100323 (Gilead Sciences), US 2013/0165489 (University of Pennsylvania), US 2014/0221378 (Japan Tobacco), US 2014/0221380 (Japan Tobacco); WO 2009/062285 (Boehringer Ingelheim); WO 2010/130034 (Boehringer Ingelheim); WO 2013/006792 (Pharma Resources), US20140221356 (Gilead Sciences), US20100143301 (Gilead Sciences) and WO 2013/091096 (Boehringer Ingelheim).

### HIV Vaccines

In certain embodiments, the IL-15v, IL-15Rα Sushi domain-IL-15v fusion protein or CD4-targeted IL-15v, as described herein, is combined or co-administered with an HIV vaccine. Examples of HIV vaccines that can be combined with an agent of this disclosure include peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus, i.e., rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA)), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as Semliki forest virus, Venezuelan equine encephalitis virus and sindbis virus (see, e.g., Lauer, et al., Clin Vaccine Immunol (2017) 24(1):e00298-16); LNP formulated mRNA based therapeutic vaccines; LNP-formulated self-replicating RNA/self-amplifying RNA vaccines.

Examples of vaccines include: AAVLP-HIV vaccine, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAd0x1.tHIVconsv1 vaccine, CMV-MVA triplex vaccine, ChAdOx1.HTI, C62-M4, Chimigen HIV vaccine, ConM SOSIP.v7 gp140, rgp120 (AIDSVAX), ALVAC HIV (vCP1521)/AIDSVAX B/E (gp120) (RV144), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, GS-1983, GS-6708, TVI-HIV-1, Ad-4 (Ad4-env Clade C+Ad4-mGag), Paxvax, EN41-UGR7C, EN41-FPA2, ENOB-HV-11, ENOB-HV-12, exoVACC, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod. HIV+MVA mosaic vaccine+gp140, AGS-004, AVX-101, AVX-201, PEP-6409, SAV-001, ThV-01, TL-01, TUTI-16, VGX-3300, VIR-1111, IHV-001, UVAX-1107, repRNA vaccine (LION nanoparticle, HIV-1), 763SIP8/MPLA-5 vaccine, BG505 SOSIP.664 gp140 adjuvanted vaccine, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, MVA.tHIVconsv3, MVA.tHIVconsv4, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, UBI HIV gp120, mRNA based prophylactic vaccines, VPI-211, HIV ANTI-CD40.ENV GP140, HIV ANTI-CD40.HIV5PEP, multimeric HIV gp120 vaccine, TBL-1203HI, CH505 TF chTrimer, CD40.HIVRI.Env vaccine, VRC-HIVRGP096-00-VP, Drep-HIV-PT-1, BG505 MD39.3 mRNA, BG505 MD39.3 gp151 CD4KO mRNA, BG505 MD39.3 gp151 mRNA, mRNA-1644, mRNA-1547, and mRNA-1574, and anti-HIV vaccines described in WO2021011544 and WO2022155258.

### HIV Long-Acting Therapy

In some embodiments, the IL-15v, IL-15Rα Sushi domain-IL-15v fusion protein or CD4-targeted IL-15v, as described herein, can be combined or co-administered with a long-acting HIV inhibitor or long-acting HIV regimen. Examples of long acting anti-HIV regimens that can be combined or co-administered with the IL-15v, IL-15Rα Sushi domain-IL-15v fusion protein or CD4-targeted IL-15v, as described herein, include, but are not limited to, lenacapavir, cabotegravir, rilpivirine, CABENUVA® (cabotegravir LA+rilpivirine LA), any integrase LA, VM-1500 LAI, VM-3500, GS-1614, maraviroc (LAI), tenofovir implant, tenofovir long acting, tenofovir prodrug long acting, islatravir, islatravir (MK-8591) subdermal implant, islatravir prodrug, doravirine, LYN-172, raltegravir, long acting raltegravir+lamivudine, XVIR-120, GSK-3739937 (long-acting), long acting dolutegravir, darunavir long acting, VOCABRIA® (cabotegravir LA), APRETUDE® (cabotegravir LAI), REKAMBYS® (rilpivirine LA), elvitegravir (extended release), T-1144, ODE-Bn-TFV, CP-112, S-648414, and transdermal devices that can deliver HIV drugs, such as transdermal tenofovir (WO2020092990).

### P-Glycoprotein Inhibitors

In some embodiments, the compounds described herein can be co-administered with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar.

### Birth Control (Contraceptive) Combination Therapy

In certain embodiments, the IL-15v, IL-15Rα Sushi domain-IL-15v fusion protein or CD4-targeted IL-15v, as described herein, are combined or co-administered with a birth control or contraceptive regimen. Therapeutic agents used for birth control (contraceptive) include cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the IL-15v, IL-15Rα Sushi domain-IL-15v fusion protein or CD4-targeted IL-15v, as described herein, are combined or co-administered with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include LB-1903, ENOB-HV-01, ENOB-HV-21, ENOB-HV-31, GOVX-B01, HSPCs overexpressing ALDHI (LV-800, HIV infection), AGT103-T, and SupT1 cell-based therapy. Examples of dendritic cell therapy include AGS-004. CCR5 gene editing agents include SB-728T. CCR5 gene inhibitors include Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with the IL-15v, IL-15Rα Sushi domain-IL-15v fusion protein or CD4-targeted IL-15v, as described herein. In some embodiments, the agents described herein are co-administered with AGT-103-transduced autologous T-cell therapy or AAV-eCD4-Ig gene therapy.

### Gene Editors

In certain embodiments, the IL-15v, IL-15Rα Sushi domain-IL-15v fusion protein or CD4-targeted IL-15v, as described herein, are combined or co-administered with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T-Cell Therapy

In some embodiments, the IL-15v, IL-15Rα Sushi domain-IL-15v fusion protein or CD4-targeted IL-15v, as described herein, can be co-administered with a population of immune effector cells engineered to express a chimeric antigen receptor (CAR), wherein the CAR comprises an HIV antigen binding domain. The HIV antigen includes an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+T-cell, a CD8+T-cell, or a combination thereof. Cells can be autologous or allogeneic. Examples of HIV CAR-T include convertible CAR-T, VC-CAR-T, CMV-N6-CART, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR-T-Cell Therapy

In certain embodiments, the IL-15v, IL-15Rα Sushi domain-IL-15v fusion protein or CD4-targeted IL-15v, as described herein, are combined or co-administered with a population of TCR-T-cells. TCR-T-cells are engineered to target HIV derived peptides present on the surface of virus-infected cells, for example ImmTAV.

### B-Cell Therapy

In certain embodiments, the IL-15v, IL-15Rα Sushi domain-IL-15v fusion protein or CD4-targeted IL-15v, as described herein, are combined or co-administered with a population of B cells genetically modified to express broadly neutralizing antibodies, such as 3BNC117 (Hartweger, et al, J. Exp. Med. (2019) 1301, Moffett, et al., Sci. Immunol. 4, eaax0644 (2019) 17 May 2019).

### Illustrative Combination Therapies

In some embodiments, the IL-15v, IL-15Rα Sushi domain-IL-15v fusion protein or CD4-targeted IL-15v, as described herein, are combined or co-administered with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; EDURANT® (dolutegravir+rilpivirine); raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the IL-15v, IL-15Rα Sushi domain-IL-15v fusion protein or CD4-targeted IL-15v, as described herein, are combined or co-administered with an HIV nucleoside or nucleotide inhibitor of reverse transcriptase and an HIV non-nucleoside inhibitor of reverse transcriptase. In another specific embodiment, the IL-15v, IL-15Rα Sushi domain-IL-15v fusion protein or CD4-targeted IL-15v, as described herein, or a pharmaceutical composition thereof, is combined or co-administered with an HIV nucleoside or nucleotide inhibitor of reverse transcriptase, and an HIV protease inhibiting compound. In an additional embodiment, the IL-15v, IL-15Rα Sushi domain-IL-15v fusion protein or CD4-targeted IL-15v, as described herein, or a pharmaceutical composition thereof, is combined or co-administered with an HIV nucleoside or nucleotide inhibitor of reverse transcriptase, an HIV non-nucleoside inhibitor of reverse transcriptase, and a pharmacokinetic enhancer. In certain embodiments, the IL-15v, IL-15Rα Sushi domain-IL-15v fusion protein or CD4-targeted IL-15v, as described herein, or a pharmaceutical composition thereof, is combined or co-administered with at least one HIV nucleoside inhibitor of reverse transcriptase, an integrase inhibitor, and a pharmacokinetic enhancer. In another embodiment, the IL-15v, IL-15Rα Sushi domain-IL-15v fusion protein or CD4-targeted IL-15v, as described herein, or a pharmaceutical composition thereof, is combined or co-administered with two HIV nucleoside or nucleotide inhibitors of reverse transcriptase.

In some embodiments, the IL-15v, IL-15Rα Sushi domain-IL-15v fusion protein or CD4-targeted IL-15v, as described herein, are combined or co-administered with abacavir sulfate, tenofovir, tenofovir disoproxil, tenofovir disoproxil fumarate, tenofovir disoproxil hemifumarate, tenofovir alafenamide, or tenofovir alafenamide hemifumarate.

In some embodiments, the IL-15v, IL-15Rα Sushi domain-IL-15v fusion protein or CD4-targeted IL-15v, as described herein, are combined or co-administered with tenofovir, tenofovir disoproxil, tenofovir disoproxil fumarate, tenofovir alafenamide, or tenofovir alafenamide hemifumarate.

In some embodiments, the IL-15v, IL-15Rα Sushi domain-IL-15v fusion protein or CD4-targeted IL-15v, as described herein, are combined or co-administered with a first additional therapeutic agent selected from the group consisting of abacavir sulfate, tenofovir, tenofovir disoproxil, tenofovir disoproxil fumarate, tenofovir alafenamide, and tenofovir alafenamide hemifumarate, and a second additional therapeutic agent selected from the group consisting of emtricitabine and lamivudine.

In some embodiments, the IL-15v, IL-15Rα Sushi domain-IL-15v fusion protein or CD4-targeted IL-15v, as described herein, are combined or co-administered with a first additional therapeutic agent selected from the group consisting of tenofovir, tenofovir disoproxil, tenofovir disoproxil fumarate, tenofovir alafenamide, and tenofovir alafenamide hemifumarate, and a second additional therapeutic agent, wherein the second additional therapeutic agent is emtricitabine.

In some embodiments, the IL-15v, IL-15Rα Sushi domain-IL-15v fusion protein or CD4-targeted IL-15v, as described herein, are combined or co-administered with a first additional therapeutic agent selected from a first additional therapeutic agent selected from dolutegravir, cabotegravir, islatravir, darunavir, bictegravir, elsulfavirine, rilpivirine, and lenacapavir and a second additional therapeutic agent selected from emtricitabine and lamivudine.

In some embodiments, the IL-15v, IL-15Rα Sushi domain-IL-15v fusion protein or CD4-targeted IL-15v, as described herein, are combined or co-administered with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In certain embodiments, the IL-15v, IL-15Rα Sushi domain-IL-15v fusion protein or CD4-targeted IL-15v, as described herein, are combined or co-administered with 5-30 mg tenofovir alafenamide fumarate, tenofovir alafenamide hemifumarate, or tenofovir alafenamide, and 200 mg emtricitabine. In certain embodiments, the IL-15v, IL-15Rα Sushi domain-IL-15v fusion protein or CD4-targeted IL-15v, as described herein, are combined or co-administered with 5-10, 5-15, 5-20, 5-25, 25-30, 20-30, 15-30, or 10-30 mg tenofovir alafenamide fumarate, tenofovir alafenamide hemifumarate, or tenofovir alafenamide, and 200 mg emtricitabine. In certain embodiments, the IL-15v, IL-15Rα Sushi domain-IL-15v fusion protein or CD4-targeted IL-15v, as described herein, are combined or co-administered with 10 mg tenofovir alafenamide fumarate, tenofovir alafenamide hemifumarate, or tenofovir alafenamide, and 200 mg emtricitabine. In certain embodiments, the IL-15v, IL-15Rα Sushi domain-IL-15v fusion protein or CD4-targeted IL-15v, as described herein, are combined or co-administered with 25 mg tenofovir alafenamide fumarate, tenofovir alafenamide hemifumarate, or tenofovir alafenamide, and 200 mg emtricitabine.

In certain embodiments, the IL-15v, IL-15Rα Sushi domain-IL-15v fusion protein or CD4-targeted IL-15v, as described herein, are combined or co-administered with 200-400 mg tenofovir disoproxil fumarate, tenofovir disoproxil hemifumarate, or tenofovir disoproxil, and 200 mg emtricitabine. In certain embodiments, the IL-15v, IL-15Rα Sushi domain-IL-15v fusion protein or CD4-targeted IL-15v, as described herein, are combined or co-administered with 200-250, 200-300, 200-350, 250-350, 250-400, 350-400, 300-400, or 250-400 mg tenofovir disoproxil fumarate, tenofovir disoproxil hemifumarate, or tenofovir disoproxil, and 200 mg emtricitabine. In certain embodiments, the IL-15v, IL-15Rα Sushi domain-IL-15v fusion protein or CD4-targeted IL-15v, as described herein, are combined or co-administered with 300 mg tenofovir disoproxil fumarate, tenofovir disoproxil hemifumarate, or tenofovir disoproxil, and 200 mg emtricitabine.

### 13. Kits

Further provided are kits comprising one or more unitary doses of the IL-15v, IL-15Rα Sushi domain-IL-15v fusion protein or CD4-targeted IL-15v, as described herein, including polynucleotides encoding, and pharmaceutical compositions and conjugates thereof.

In some embodiments, the kits comprise one or more containers (e.g., vials, ampules) comprising one or more of IL-15v, IL-15Rα Sushi domain-IL-15v fusion protein or CD4-targeted IL-15v, as described herein, polynucleotides encoding such IL-15v, IL-15Rα Sushi domain-IL-15v fusion protein or CD4-targeted IL-15v, LNPs or pharmaceutical compositions containing such IL-15v, IL-15Rα Sushi domain-IL-15v fusion protein or CD4-targeted IL-15v. Further provided are kits comprising one or more unitary doses of the IL-15v, IL-15Rα Sushi domain-IL-15v fusion protein or CD4-targeted IL-15v, as described herein, in one or more pre-loaded syringes.

In one embodiment, kits comprise one or more unitary doses of the IL-15v, IL-15Rα Sushi domain-IL-15v fusion protein or CD4-targeted IL-15v, as described herein, in combination with one or more (e.g., one, two, three, one or two, or one to three) additional therapeutic agents. In some embodiments, the kits comprise one or more unitary doses of the one or more unitary doses of the IL-15v, IL-15Rα Sushi domain-IL-15v fusion protein or CD4-targeted IL-15v, as described herein, or the polynucleotide or polynucleotides encoding, in one or more containers. In some embodiments, the kits comprise one or more unitary doses of the one or more unitary doses of the IL-15v, IL-15Rα Sushi domain-IL-15v fusion protein or CD4-targeted IL-15v, as described herein, and a second agent for treating an HIV infection in separate containers.

In some embodiments, the kits further comprise one or more unitary doses of a toll-like receptor (TLR) agonist. In some embodiments, the TLR agonist is a TLR7 agonist or a TLR8 agonist. In some embodiments, the TLR7 agonist is selected from the group consisting of vesatolimod, imiquimod, and resiquimod. In some embodiments, the kits comprise one or more unitary doses of the IL-15v, IL-15Rα Sushi domain-IL-15v fusion protein or CD4-targeted IL-15v, as described herein, and one or more anti-HIV broadly neutralizing antibodies (bNAbs).

In some embodiments, the kits further comprise one or more unitary doses of a bNAb that binds to an epitope within HIV gp120 V3 glycan domain and competes with or comprises VH and VL regions from an antibody selected from the group consisting of zinlirvimab (GS-2872; 10-1074-LS), elipovimab (GS-9722), PGT-121, PGT-121.66, PGT-121.414, PGT-122, PGT-123, PGT-124, PGT-125, PGT-126, PGT-128, PGT-130, PGT-133, PGT-134, PGT-135, PGT-136, PGT-137, PGT-138, PGT-139, 10-1074, 10-1074-J, VRC24, 2G12, BG18, 354BG8, 354BG18, 354BG42, 354BG33, 354BG129, 354BG188, 354BG411, 354BG426, DH270.1, DH270.6, PGDM12, VRC41.01, PGDM21, PCDN-33A, BF520.1 and VRC29.03. In some embodiments, the kits further comprise one or more unitary doses of a bNAb that binds to an epitope within HIV gp120 V3 glycan domain and competes with or comprises VH and VL regions from an antibody selected from the group consisting of zinlirvimab (GS-2872; 10-1074-LS), elipovimab (GS-9722), PGT-121, PGT-121.414, 10-1074, 10-1074-J and PGT-134.

In some embodiments, the kits further comprise one or more unitary doses of a bNAb that binds to an epitope within HIV gp120 CD4 binding site, and competes with or comprises VH and VL regions from an antibody selected from the group consisting of 3BNC117, GS-9723, teropavimab (GS-5423; 3BNC117-LS), 3BNC60, b12, F105, VRC01, VRC07, VRC07-523, VRC03, VRC06, VRC06b01 VRC08, VRC0801, NIH45-46, PGV04 (a.k.a., VRC-PG04); CH103, 44-VRC13.01, 1NC9, 12A12, N6, 1-18, N49-P7, NC-Cow1, IOMA, CH235 and CH235.12, N49P6, N49P7, N49P11, N49P9 and N60P25. In some embodiments, the kits further comprise one or more unitary doses of a bNAb that binds to an epitope within HIV gp120 CD4 binding site, and competes with or comprises VH and VL regions from an antibody selected from the group consisting of 3BNC117, GS-9723, VRC07 or VRC07-523.

In some embodiments, the kits comprise two or more unitary doses of IL-15v, IL-15Rα Sushi domain-IL-15v fusion protein or CD4-targeted IL-15v, as described herein, and optionally, one or more anti-HIV bNAbs, wherein the unitary doses are the same. In some embodiments, the kits comprise two or more unitary doses of IL-15v, IL-15Rα Sushi domain-IL-15v fusion protein or CD4-targeted IL-15v, as described herein, and optionally, one or more anti-HIV bNAbs, wherein the unitary doses are different.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside or nucleotide inhibitor of reverse transcriptase. In a specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside or nucleotide inhibitor of reverse transcriptase and an HIV non-nucleoside inhibitor of reverse transcriptase. In another specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside or nucleotide inhibitor of reverse transcriptase, and an HIV protease inhibiting compound. In an additional embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside or nucleotide inhibitor of reverse transcriptase, an HIV non-nucleoside inhibitor of reverse transcriptase, and a pharmacokinetic enhancer. In certain embodiments, the kit includes an IL-15v, IL-15Rα Sushi domain-IL-15v fusion protein or CD4-targeted IL-15v, as described herein, polynucleotide encoding or pharmaceutical composition comprising, and at least one HIV nucleoside inhibitor of reverse transcriptase, an integrase inhibitor, and a pharmacokinetic enhancer. In another embodiment, the kit includes an IL-15v, IL-15Rα Sushi domain-IL-15v fusion protein or CD4-targeted IL-15v, as described herein, polynucleotide encoding or pharmaceutical composition comprising, and two HIV nucleoside or nucleotide inhibitors of reverse transcriptase. In a specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside or nucleotide inhibitor of reverse transcriptase and an HIV capsid inhibitor. In a specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV nucleoside inhibitor of reverse transcriptase and an HIV capsid inhibitor. In a specific embodiment, the kit includes an IL-15v, IL-15Rα Sushi domain-IL-15v fusion protein or CD4-targeted IL-15v, as described herein, polynucleotide encoding or pharmaceutical composition comprising, and an HIV capsid inhibitor. In a specific embodiment, the kit includes an IL-15v, IL-15Rα Sushi domain-IL-15v fusion protein or CD4-targeted IL-15v, as described herein, polynucleotide encoding or pharmaceutical composition comprising, and one, two, three or four HIV bNAbs. In a specific embodiment, the kit includes an IL-15v, IL-15Rα Sushi domain-IL-15v fusion protein or CD4-targeted IL-15v, as described herein, polynucleotide encoding or pharmaceutical composition comprising, and one, two, three or four HIV bNAbs and an HIV capsid inhibitor. In a specific embodiment, the kit includes an IL-15v, IL-15Rα Sushi domain-IL-15v fusion protein or CD4-targeted IL-15v, as described herein, polynucleotide encoding or pharmaceutical composition comprising, and one, two, three or four HIV bNAbs, an HIV capsid inhibitor, and an HIV nucleoside inhibitor of reverse transcriptase.

In one embodiment, the kit comprises one or more pharmaceutical packs comprising one or more containers (e.g., vials, ampules) containing one or more of the ingredients of the pharmaceutical compositions described herein, such as one or more IL-15v, IL-15Rα Sushi domain-IL-15v fusion protein or CD4-targeted IL-15v, as provided herein. In some instances, the kits contain a pharmaceutical composition described herein. In one embodiment, kits comprising an IL-15v, IL-15Rα Sushi domain-IL-15v fusion protein or CD4-targeted IL-15v, as described herein, or a pharmaceutical composition thereof, in combination with one or more (e.g., one, two, three, one or two, or one to three) additional therapeutic agents (such as those disclosed above) are provided.

Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

The following examples are offered to illustrate, but not to limit the claimed invention.

### Example 1

### Design of CD4-Targeted IL-15 Variant Fc Fusion Proteins

In this example, we assessed IL-15 fusion molecules to evaluate potency, selectivity and manufacturability. Several characteristics were selected for, including sufficient pharmacokinetics to achieve the desired pharmacodynamic effect with a single administration, appropriate bispecific chain pairing, reduced FcR binding and high selectivity.

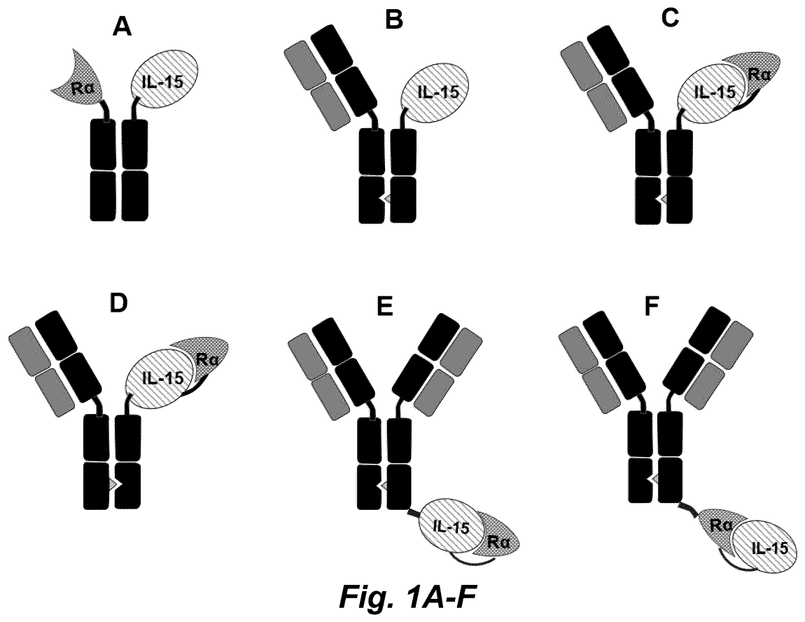

To achieve sufficient pharmacokinetic properties, IL-15 was fused via a linker domain to an antibody Fc domain. To facilitate targeting, fusion molecules were designed that had the Fab domain of a CD4-specific antibody and an IL-15 domain. Various formats are depicted in FIG. 1.

Amino acid substitutions were introduced into the Fc domain to reduce or minimize FcγR engagement, reducing or eliminating undesired cytotoxicity when the molecule binds target cells. In this example, molecules are characterized that contain the L234A and L235A (Eu numbering) Fc amino acid substitutions to reduce or eliminate FcγR binding (Chappel et al., Proc Natl Acad Sci USA (1991) 88(20):9036-40). The P331S or G237A amino acid substitutions can also be used to reduce or eliminate C1q binding (Xu et al., J Biol Chem (1994) 269:3469-74; Hale et al., MAbs (2024) 16(1):2402701).

Endogenous human IL-15 is typically presented in trans (Dubois et al., Immunity (2002) 17:537-547) by IL-15Rα. IL-15Rα can stabilize and enhance the activity of IL-15 (Bergamashi et al., J Biol Chem (2008) 283:4189-4199). The Sushi domain of IL-15Rα is the domain that specifically binds to IL-15 (Wei et al., J Immunol (2001) 167:277-283). We tested molecules with (e.g. Molecules 239 and 241, FIG. 1C) and without the IL-15Rα Sushi domain (e.g., Molecules 242 and 243, FIG. 1B). Molecules without the IL-15Rα Sushi domain had decreased yield after purification and decreased activity relative to molecules containing the IL15Rα Sushi domain. Data are summarized in Tables 1 and 2.

Peripheral blood mononuclear cell (PBMC)-based proliferation assay. IL-15 fusion molecule potency and selectivity for CD4+ T cells, CD8+ T cells and NK cells was assessed in a proliferation assay. Human PBMCs from multiple donors were freshly isolated and resuspended in RPMI-1640, 10% heat inactivated fetal bovine serum, 1× penicillin streptomycin and seeded at 250,000 cells per well in a 96 well U-bottom plate and added to serial dilutions of the molecule. The mixture was incubated at 37° C. for 5 days. Cells were washed in 1×PBS and labeled with live/dead stain and incubated for 10 minutes. Cells were washed in 1×PBS with 2% heat inactivated fetal bovine serum before being incubated in cell surface antibodies CD3-AF700, CD4-PerP-Cy5.5, CD8-APC-H7, CD16-PE, CD56-APC and incubated for 20 minutes. Cells were washed and fixed and permeabilized in BD Cytofix/Cyto Perm buffer for 20 minutes and washed in 1×CytoPerm buffer. Cells were incubated in Ki-67-BV421 antibody for 30 minutes at room temperature for 30 minutes, washed in 1×CytoPerm and analyzed by flow cytometry using a LSR Fortessa X-20 (BD Biosciences) for Ki67 levels on CD4+ T cells (CD3+CD4+), CD8+ T cells (CD3+CD8+) or NK cells (CD3-CD16+CD56+). Ki67 was used as a marker of proliferation, which is stimulated by IL-15 activation. Results were analyzed using FlowJo V10 and GraphPad Prism using four parameter variable slope dose response curves to determine the half maximal effective concentration (EC50). The mean EC50 values (nM) of biological replicates are summarized in Table 1.

TABLE 1
EC50 Values for Proliferation of CD4+ T Cells, CD8+ T Cells and NK Cells
Induced by IL-15-Fc Variants with or without CD4-Targeting and the IL-15Rα Sushi Domain

| | | | | | CD4 | CD8 | NK |
|---|---|---|---|---|---|---|---|
| | | CD4 | | | EC50 | EC50 | EC50 |
| Molecule | Fab Name | domain | IL-15 mutein | Fc | (nM) | (nM) | (nM) |
| 234 | NA | NA | WT | IgG1 | 0.71 | 0.36 | 0.047 |
| 263 | NA | NA | rhesus WT | rhesus | 0.68 | 0.31 | 0.044 |
| | | | | IgG1 | | | |
| 235 | NA | NA | N65D | IgG1 | 11 | 6.0 | 1.1 |
| 236 | NA | NA | N4D/N65D | IgG1 | 16 | 16 | 2.1 |
| 237 | Palivizumab | D1 | N65D | IgG1 | >1000 | >1000 | 16 |
| 238 | M1 (mouse) | D1 | N4D/N65D | IgG1 | >1000 | >1000 | >1000 |
| 239 | M1 (mouse) | D1 | WT | IgG1 | 0.012 | 7.3 | 0.63 |
| 240 | M1 (mouse) | D1 | N65D | IgG1 | 0.21 | >1000 | >1000 |
| 241 | M1 (mouse) | D1 | N4D/N65D | IgG1 | 0.13 | >1000 | >1000 |
| 242 | M1 (mouse) | D1 | WT | IgG1 | >1000 | >1000 | >1000 |
| 243 | M1 (mouse) | D1 | N4D/N65D | IgG1 | >1000 | >1000 | 23 |

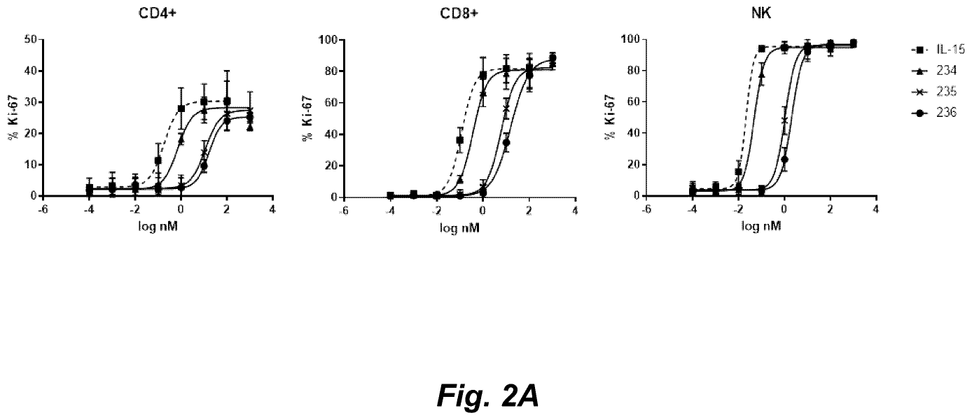

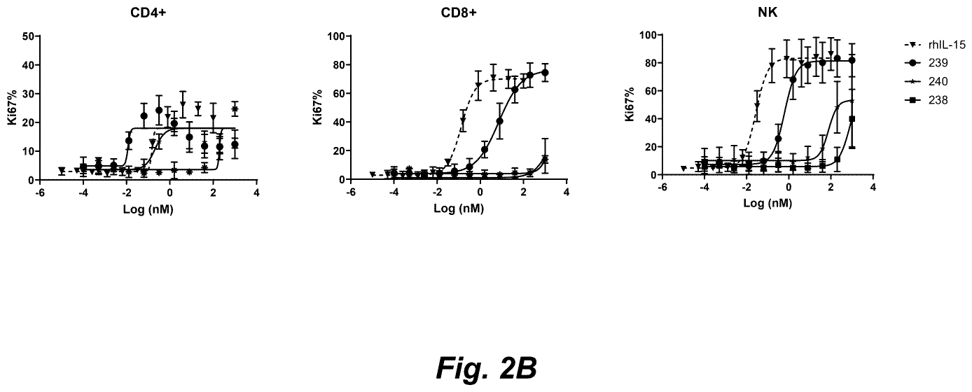

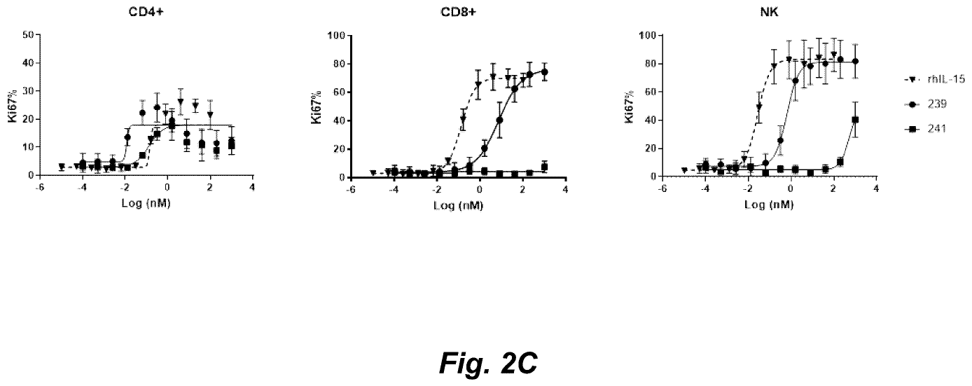

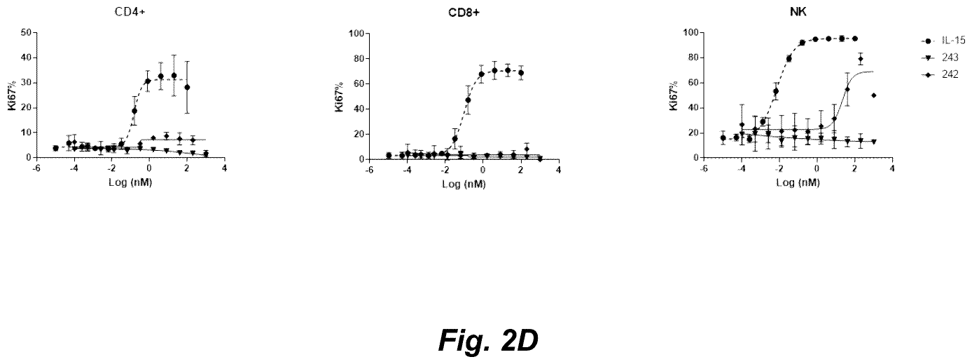

The data in Table 1 and FIG. 2 demonstrate a range of potencies for IL-15 fusion proteins for Ki67 activation in PBMCs. Molecules without targeting domains (e.g., Molecules 234-238, FIG. 1A) induced Ki67 activation in NK cells with EC50s ranging from 0.047 to 140 nM. These molecules each had greater potency in NK cells than in CD4+ T cells, with CD4 EC50s ranging from 0.71 to >1000 nM. Molecules without the IL-15Rα Sushi domain had CD4 EC50s >1000 nM, indicating a lack of the desired potency in the absence of this domain. Molecules with a CD4-specific Fab on one arm (i.e., antigen binding domain) and IL-15 with the Sushi domain of IL-15Rα on the other arm (Molecules 239-241, FIG. 1C) had the greatest activity in CD4 T cells, with EC50s ranging from 0.012 to 0.21 nM. For these molecules, NK cell EC50s ranged from 0.63 to >1000 nM. Molecules with the CD4-specific Fab and a variant of IL-15 that contains the N65D amino acid substitution had NK cell EC50s of >1000 nM. These molecules had much lower potency inducing proliferation of NK cells and CD8+ T cells in comparison to molecules that contained wild type IL-15 (e.g., Molecule 239). IL-15 amino acid substitution N65D has previously been shown to reduce IL-15 activity (Zhu, et al., J Immunol (2009) 183(6):3598-3607). In this example, IL-15 muteins with reduced activity show greater selectivity for CD4+ T cells than NK cells when they are used with CD4-specific targeting arms (i.e., antigen binding domains).

Expression and purification. For expression, sequences encoding Fc-IL-15 or CD4 specific Fab-IL-15 variant fusion proteins were codon biased for expression in CHO cells and transiently expressed in Expi293™ cells according to the manufacturer's protocol. Clarified supernatant was harvested and purified via a two-step purification process comprising protein A first followed up by cation exchange chromatography (CIEX), e.g., using an ÄKTA FPLC (Cytiva Life Sciences), enclosed within a 4.0° C. chromatography chamber. Antibody containing supernatant was loaded to a MabSelect Sure (Cytiva Life Sciences) column. The column was washed with a low salt buffer (25 mM Tris HC1 pH 7.5, 25 mM NaCl) for 10 column volumes (CV), then with a high salt buffer (25 mM Tris HC1 pH 7.5, 500 mM NaCl) for 5 CV to remove non-specific binding, and finally with a low salt wash (25 mM Tris HC1 pH 7.5, 25 mM NaCl) for 10 CV. To elute the antibody, an isocratic elution step of 100 mM Sodium Acetate pH 3.5 was used. The eluted pool was neutralized to pH 6.0 with 1 M Tris HC1 pH 9.0, and subjected to CIEX purification, HiTrap SP HP (Cytiva Life Sciences) in gradient elution to further remove impurities. The CD4-targeted IL-15v protein was dialyzed into histidine buffer supplemented with sucrose, sterile filtered, and stored at 4° C. Concentration of the formulated protein was measured by A280. Purity was assessed as percent monodispersed by analytical size-exclusion chromatography. Identity of the protein was determined by mass spectrometry. The results are summarized in Table 2.

TABLE 2
Expression and Purification Yields of Bispecific
Molecule with or without the IL-15Rα Sushi Domain

| | | | | | Purified |
|---|---|---|---|---|---|
| | | CD4 | IL-15 | | yield |
| Molecule | Fab Name | domain | mutein | Fc | (mg/L) |
| 239 | mouse M1 | D1 | WT | IgG1 | 8 |
| 241 | mouse M1 | D1 | N4D/N65D | IgG1 | 17 |
| 242 | mouse M1 | D1 | WT | IgG1 | 2.1 |
| 243 | mouse M1 | D1 | N4D/N65D | IgG1 | 0.5 |

Among the 4 Fc-IL-15v described in Table 2, the two molecules containing the IL-15Rα Sushi domain (Molecules 239 and 241; FIG. 1C) ranged from 8-17 mg/L. The two molecules lacking the IL-15Rα Sushi domain (Molecules 242-243; FIG. 1B) gave a much poorer yield, ranging from 8-17 mg/L. These molecules appeared to have reduced expression and/or increased propensity for aggregation.

Correct pairing of heterodimeric molecules can be facilitated by use of “knob-in-hole” heterodimeric Fc domains (Atwell, et al., J Mol Biol (1997) 270(1):26-35). The ‘hole’ amino acid substitutions (T366S, L368A and Y407V) are incorporated into one Fc polypeptide chain, while the T366W ‘knob’ amino acid substitution is incorporated into the other Fc polypeptide chain. Co-transfection of such constructs leads to preferential formation of the desired heterodimer and decreased levels of homodimer contaminants. Positions of Fc domain amino acid substitutions are according to Eu numbering.

Developability can be improved by introducing a C220S amino acid substitution in the hinge region of the IL-15 arm. This eliminates a free cysteine that is typically responsible for forming a disulfide bond with a corresponding cysteine in the light chain. As the IL-15 arm does not have a light chain, the free cysteine can be removed to reduce potential reactivity.

Further improvements to the purification process can be achieved by introducing amino acid substitutions that favor heterodimeric molecules and reduce contaminating homodimeric products. The H435R or H435R+Y436F amino acid substitutions (ProA knock-out (KO)) may be used in either of the Fc-containing chains to reduce Protein A binding (Jendeberg et al., J Immunol Methods (1997) 201:25.34). In the examples provided, the ProA KO amino acid substitutions were typically used in combination with the hole amino acid substitutions, as homodimers of this chain are more likely to form over knob homodimers. Incorporating ProA KO amino acid substitutions on the same Fc polypeptide chain as the hole amino acid substitutions reduces binding of the hole-containing homodimers to Protein A and simplify the purification of the desired heterodimeric molecule. In the cases where the Fab domain contains a VH3 family member (known to bind Protein A in addition to the Fc), additional amino acid substitutions can be incorporated to reduce binding of such a Fab domain to Protein A as well. In examples provided, G65S and Q81E amino acid substitutions were introduced to reduce Fab binding to Protein A (e.g. Molecule 100).

CD4-targeted IL-15v molecules were designed with similar CD4-specific Fab arms and IL-15 arms, but with different orientations of the knob-in-hole amino acid substitutions to determine the effect on potency and selectivity. Expression and purification of these molecules was performed as above. Yields were not significantly different when the knob was on either the IL-15 arm (FIG. 1C) or the CD4-specific Fab arm (FIG. 1D). The results are summarized in Table 3. The molecules were also tested in the PBMC proliferation assay as described above. The results shown in Table 4 indicate consistent potency in CD4 T cell proliferation with knob on either the IL-15 arm (FIG. 1C) or the CD4-specific Fab arm (FIG. 1D).

TABLE 3
Expression and Purification Yields of Bispecific
Molecule with Knob-in-Hole Orientations

| | | | | | Purified |
|---|---|---|---|---|---|
| | | CD4 | IL-15 | | yield |
| Molecule¹ | Fab Name | domain | mutein | Fc | (mg/L) |
| 111 | 12 (humanized M1) | D1 | N65D | IgG1 | 91 |
| | (38Q/43G/53E) | | | | |
| 100 | 7 (humanized M1) | D1 | N65D | IgG1 | 30 |
| | D ProA | | | | |
| 113 | 12 (humanized M1) | D1 | N65D | IgG1 | 45 |
| 240 | mouse M1 | D1 | N65D | IgG1 | 15 |
| 244 | mouse M1 | D1 | N65D | IgG1 | 13 |

¹Amino acid sequences of Molecules 100-233, 245, 246, 249, 251-255 and 259-262 are provided in Table G. VH/VL sequences of the humanized Fabs are in Table B.

TABLE 4
EC50 Values for Proliferation of CD4+ Cells, CD8+ Cells and NK Cells
Induced by IL-15-Fc Variants with Different Knob-in-Hole Orientations

| | | | | | CD4 | CD8 | NK |
|---|---|---|---|---|---|---|---|
| | | CD4 | | | EC50 | EC50 | EC50 |
| Molecule | Fab Name | domain | IL-15 mutein | Fc | (nM) | (nM) | (nM) |
| 111 | 12 (humanized M1) | D1 | N65D | IgG1 | 0.044 | >1000 | 99 |
| | (38Q/43G/53E) | | | | | | |
| 100 | 7 (humanized M1) D ProA | D1 | N65D | IgG1 | 0.056 | >1000 | 54 |
| 113 | 12 (humanized M1) | D1 | N65D | IgG1 | 0.022 | >1000 | 35 |
| 240 | mouse M1 | D1 | N65D | IgG1 | 0.19 | >1000 | >1000 |
| 244 | mouse M1 | D1 | N65D | IgG1 | 0.094 | >1000 | 140 |

In considering potential homodimer contaminants, clinical data indicate that IL-15 arm variant homodimers are less desirable than CD4 arm homodimers. Untargeted IL-15 molecules are associated with multiple adverse events in clinical studies at doses of 3 g/kg (Conlon, et al., J Clin Onc (2015) 33:74-82). By contrast, CD4-specific antibodies have shown good safety profiles in clinical studies at doses up to 25 mg/kg (Jacobsen et al., Antimicrob Agents Chemo (2009) 53:450-7). As knob-knob homodimers are less favorable than hole-hole homodimers, the knob was typically placed on the IL-15 arm to reduce or minimize IL-15 homodimer formation (FIG. 1C).

When designing targeted cytokines, both N- and C-terminal Fc fusions could be effective. As depicted in Table 1, effective targeting to a cell type containing the desired receptor requires reduced cytokine binding. To reduce relative IL-15 binding to IL-2Rβγ, CD4-targeted IL-15v molecules were designed with a single copy of IL-15 and not two or more copies of IL-15. This single copy of IL-15 was fused to either the N-terminus or the C-terminus of the Fc chain (FIGS. 1C-1F). In this example, N-terminal Fc-fusions of IL-15 with the Sushi domain of IL-15Rα were paired with a single CD4-specific Fab (e.g. Molecule 175, IL-15Rα-IL-15v-Fc (w/knob); FIG. 1C). Unexpectedly, Fc-fusions in which the Sushi domain of IL-15Rα was linked to IL-15 and IL-15 was linked to the C-terminus of the Fc domain (Fc-IL-15v-IL-15Rα; FIG. 1E) were found to be poorly expressed and/or purified (yields <1 mg/L). C-terminal Fc-fusions in which the IL-15 variant was linked to the Sushi domain of IL-15Rα and the sushi domain of IL-15Rα was linked to the C-terminus of the Fc domain (Fc-IL-15Rα-IL-15v; FIG. 1F) produced good yields. The results are summarized in Table 5.

TABLE 5
Expression and Purification Yields of IL-15-Fc
Variants Linked to the Fc N- or C-Terminus

| | | | | | Purified |
|---|---|---|---|---|---|
| | | CD4 | IL-15 | | yield |
| Molecule | Fab Name | domain | mutein | Fc | (mg/L) |
| 232 | bivalent 1.23 | D3 | N65D/I68A | IgG4 | 5.5 |
| 181 | 1.23 LC C36Y | D3 | N65D/I68A | IgG4 | 82 |
| 223 | bivalent 1.22 | D3 | N65D/I68A | IgG4 | 3.9 |
| 175 | 1.22 | D3 | N65D/I68A | IgG4 | 77 |

Molecules with N-terminal and C-terminal fusions were tested in the PBMC proliferation assay as described above. The results are shown in Table 6. Molecules with C-terminal fusions (e.g., Molecules 232 and 223, FIG. 1F) were found to have greater potency in PBMCs, with EC50 values ranging from 0.029 to 0.036 nM for CD4+ T cells and 49-130 nM for NK cells. In contrast, N-terminal fusions (e.g., Molecules 181 and 175, FIG. 1C) had EC50 values ranging from 0.49-4.2 nM and NK cell EC50 values ranging from 660-780 nM. As described in Example 11, molecules with greater potency are cleared more quickly in vivo and may not have the desired pharmacokinetic profile.

TABLE 6
EC50 Values for Proliferation of CD4+ Cells, CD8+ Cells and NK Cells
Induced by IL-15-Fc Variants Linked to the Fc N- or C-Terminus

| | | | | | CD4 | CD8 | NK |
|---|---|---|---|---|---|---|---|
| | | CD4 | | | EC50 | EC50 | EC50 |
| Molecule | Fab Name | domain | IL-15 mutein | Fc | (nM) | (nM) | (nM) |
| 232 | bivalent 1.23 | D3 | N65D/I68A | IgG4 | 0.036 | 400 | 49 |
| 181 | 1.23 LC C36Y | D3 | N65D/I68A | IgG4 | 4.2 | >1000 | 780 |
| 223 | bivalent 1.22 | D3 | N65D/I68A | IgG4 | 0.029 | 300 | 130 |
| 175 | 1.22 | D3 | N65D/I68A | IgG4 | 0.49 | >1000 | 660 |

Multiple human IgG subclasses could be used to provide the Fc domain for a targeted cytokine. These subclasses can have different degrees of flexibility (Roux et al., J Immunol (1997) 159:3372-3382). In this example, the CD4-specific Fab and IL-15 variants are fused to either IgG1 or IgG4 Fc domains. Molecules were designed with various CD4-specific Fabs that bound different domains of CD4. These molecules were evaluated in the PBMC proliferation assay described above. The results indicate that IgG4-based Fc fusion molecules are consistently more potent in stimulating proliferation of CD4+ T cells and NK cells than IgG1-based Fc fusion molecules. The results are shown in Table 7.

TABLE 7
EC50 Values for Proliferation of CD4+ Cells, CD8+ Cells and NK
Cells Induced by IL-15-Fc Variants with IgG1 or IgG4 Fc Domains

| | Fab | CD4 | IL-15 | | CD4 | CD8 | NK |
|---|---|---|---|---|---|---|---|
| Molecule | Name | domain | mutein | Fc | EC50 (nM) | EC50 (nM) | EC50 (nM) |
| 111 | 12 | D1 | N65D | IgG1 | 0.044 | >1000 | 99 |
| 112 | 12 | D1 | N65D | IgG4 | 0.0029 | >1000 | 15 |
| 100 | 7 ΔProA | D1 | N65D | IgG1 | 0.056 | >1000 | 54 |
| 101 | 7 ΔProA | D1 | N65D | IgG4 | 0.0023 | >1000 | 11 |
| 135 | 2.9 | D2 | N65D/I68A | IgG1 | >1000 | >1000 | >1000 |
| 136 | 2.9 | D2 | N65D/I68A | IgG4 | 0.083 | >1000 | 570 |
| 141 | 2.11 | D2 | N65D/I68A | IgG1 | 1.2 | >1000 | 420 |
| 142 | 2.11 | D2 | N65D/I68A | IgG4 | 1.8 | >1000 | >1000 |
| 155 | 2.15 | D2 | N65D/I68A | IgG1 | >1000 | >1000 | >1000 |
| 156 | 2.15 | D2 | N65D/I68A | IgG4 | 0.39 | >1000 | 650 |
| 170 | 1.21 | D3 | N65D/I68A | IgG1 | 4.8 | >1000 | >1000 |
| 171 | 1.21 | D3 | N65D/I68A | IgG4 | 0.77 | >1000 | 630 |
| 174 | 1.22 | D3 | N65D/I68A | IgG1 | 2.2 | >1000 | >1000 |
| 175 | 1.22 | D3 | N65D/I68A | IgG4 | 0.49 | >1000 | 660 |
| 180 | 1.23 | D3 | N65D/I68A | IgG1 | 6.9 | >1000 | >1000 |
| 181 | 1.23 | D3 | N65D/I68A | IgG4 | 4.2 | >1000 | 780 |
| 185 | 1.24 | D3 | N65D/I68A | IgG1 | 0.58 | >1000 | 730 |
| 186 | 1.24 | D3 | N65D/I68A | IgG4 | 0.12 | >1000 | 410 |
| 178 | 1.23 | D3 | N65D/S7G | IgG1 | 13 | >1000 | 810 |
| 182 | 1.23 | D3 | N65D/S7G | IgG4 | 0.88 | >1000 | 160 |

### Example 2

### CD4 Binding Domain Discovery and Epitope Identification

Antibodies binding to CD4 antigens were generated using in vivo immunization of Trianni human Ig transgenic mice. Mice were alternately immunized with a human CD4 (hCD4) extracellular domain and a cynomolgus CD4 (cCD4) extracellular domain, both with a C-terminal His tag to allow purification (SEQ ID NOs: 1209 and 1210, respectively, in Table 8). This alternating immunization strategy was designed to select for antibodies that bind both human and cynomolgus CD4. This cross-species affinity is desirable because it enables the evaluation of pharmacokinetics, pharmacodynamics, and safety in non-clinical studies. Animals were inoculated with 80 μg of each protein in SIGMA ADJUVANT SYSTEM® (Sigma-Aldrich, St. Louis, MO) over a period of 35 days using a rapid immunization protocol (Antibody Solutions, Santa Clara, CA). The lymph nodes were harvested on day 35 and single cell suspensions enriched for IgG secreting plasma cells was generated using a CD138 positive cell selection kit (Miltenyi, Auburn, CA). A panel of CD4 antibodies that bound to CD4 were isolated using AbTheneum screening platform (Single Cell Technology, Santa Clara, CA) according to the procedure in U.S. Pat. No. 9,328,172. Plasma cells were screened for binding to human CD4, cynomolgus CD4 and human CD4 D1.22 (Chen, et al., J Virol (2014) 88:1125-1139). Paired VH and VL CD4 antibody gene fragments were cloned into a pcDNA3.1 vector containing the human IgG1 heavy chain constant region and the human kappa light chain constant region. From this, we isolated 71 clones that bound human CD4 domain 1 and 37 clones that bound human CD4 domains 2-4. Cloned antibodies were transfected into Expi293 cells (Cat: A14635, Thermofisher) to produce antibody preparations in a full-IgG format.

TABLE 8
Amino Acid Sequences of CD4 Immunogens

| | SEQ ID | |
|---|---|---|
| Name | NO: | Amino acid sequence |
| hCD4 | 1209 | KKVVLGKKGDTVELTCTASQKKSIQFHWKNSNQIKILGNQGSFLTK |
| ECD | | GPSKLNDRADSRRSLWDQGNFPLIIKNLKIEDSDTYICEVEDQKEE |
| | | VQLLVFGLTANSDTHLLQGQSLTLTLESPPGSSPSVQCRSPRGKNI |
| | | QGGKTLSVSQLELQDSGTWTCTVLQNQKKVEFKIDIVVLAFQKASS |
| | | IVYKKEGEQVEFSFPLAFTVEKLTGSGELWWQAERASSSKSWITFD |
| | | LKNKEVSVKRVTQDPKLQMGKKLPLHLTLPQALPQYAGSGNLTLAL |
| | | EAKTGKLHQEVNLVVMRATQLQKNLTCEVWGPTSPKLMLSLKLENK |
| | | EAKVSKREKAVWVLNPEAGMWQCLLSDSGQVLLESNIKVLPTWGGG |
| | | GSHHHHHHHH |
| CCD4 | 1210 | KKVVLGKKGDTVELTCNASQKKNTQFHWKNSNQIKILGIQGSFLTK |
| ECD | | GPSKLSDRADSRKSLWDQGCFSMIIKNLKIEDSDTYICEVENKKEE |
| | | VELLVFGLTANSDTHLLEGQSLTLTLESPPGSSPSVKCRSPGGKNI |
| | | QGGRTLSVPQLERQDSGTWTCTVSQDQKTVEFKIDIVVLAFQKASS |
| | | TVYKKEGEQVEFSFPLAFTLEKLTGSGELWWQAERASSSKSWITFD |
| | | LKNKEVSVKRVTQDPKLQMGKKLPLHLTLPQALPQYAGSGNLTLAL |
| | | EAKTGKLHQEVNLVVMRATQFQENLTCEVWGPTSPKLTLSLKLENK |
| | | GTTVSKQAKAVWVLNPEAGMWQCLLSDSGQVLLESNIKVVPTWGGG |
| | | GSHHHHHHHH |

The native ligand of CD4 is the MHC II molecule. Domain 1 (D1) of CD4 also mediates binding to MHC II. It is possible that a CD4-binding molecule that binds domain 1 could reduce CD4-MHC interactions and compromise immunity. A CD4-specific molecule that preserves endogenous immunity is preferable for HIV activation.

As CD4 is the natural ligand for HIV, several therapeutics have been described that use recombinant CD4 to bind HIV Env on virions or infected cells. As HIV binds domain 1 (D1) of CD4, several of these approaches specifically use recombinant CD4 domain 1 or CD4 domain 1 and 2 to engage HIV Env (Gardner et al., Nature (2015) 519:87-91; Chen et al., J Virol (2014) 88:1125-1139; Anthony-Gonda et al., Sci Transl Med (2019) 11:eaav5685; Zhen et al., PLOS Path (2021) 17:e1009404). It is desirable to design a CD4-targeted IL-15v molecule that would be compatible in combination therapies with molecules that use recombinant CD4 extracellular domain (ECD) 1 or CD4 extracellular domains 1 and 2.

For at least these two reasons, molecules that bind to CD4 domain 3 (D3) are desirable. However, previous studies have indicated that domains 1 and 2 are more exposed to antibody binding and that it is more difficult to elicit antibodies to CD4 domains 3 or 4 (Sattentau et al., J Exp Med (1989) 170:1319-1334). Our work described herein confirms Sattentau's finding, as CD4 domain 1 (D1) binding antibodies were the more common than those binding to other domains. This antibody screening strategy was specifically designed to identify antibodies that bound human and cynomolgus CD4 and did not bind human CD4 D1.22.

Antibodies that were predicted to bind human CD4 domain 1 (D1) were further screened by enzyme-linked immunosorbent assay (ELISA) for binding to human CD4 domain 1 fused to an Fc region or His-tagged human CD4 ECD (SEQ ID NO: 1209, Table 9). Briefly, 384-well white Maxisorp plates were coated with a solution of bovine serum albumin (BSA), human CD4 Doman 1-Fc, or human CD4 ECD-His protein at 2 μg/mL in carbonate-bicarbonate buffer overnight at 4° C. After removing the coating solution, nonspecific binding sites were blocked by adding 2% BSA in phosphate buffered saline with Tween 20 (PBS-T). Following the wash with PBS-T, diluted test samples in 0.2% BSA-PBS-T were added to each well and incubated for 60 minutes at room temperature with shaking. Plates were rewashed, and anti-human Kappa HRP was added at 1:15,000 dilution in 0.2% BSA-PBS-T for 1 hour at room temperature. Plates were washed, and then chemiluminescent substrate solution was added and incubated for 10 minutes. Finally, the plates were read using an Envision reader to quantify the luminescent signal. ELISA results with antibodies that were predicted to bind domain 1 of human CD4 confirmed that these antibodies were specific for domain 1 (Table 9).

TABLE 9
Antibody Binding to Human CD4 Domain 1

| | Anti-CD4 | HuCD4.D1Fc | HuCD4 ECD- | | HuCD4.D1Fc | HuCD4 ECD-His |
|---|---|---|---|---|---|---|
| Molecule | Fab | (RLU)¹ | His (RLU) | BSA | (Fold over BSA) | (Fold over BSA) |
| 115 | SCT1.17 | 132530520 | 106757360 | 9164560 | 14 | 12 |
| 116 | SCT1.25 | 9617480 | 2514280 | 440080 | 22 | 6 |
| 117 | SCT1.26 | 4021360 | 964400 | 285440 | 14 | 3 |
| 118 | SCT1.27 | 2487960 | 320320 | 291440 | 9 | 1 |
| 119 | SCT1.28 | 4008920 | 5226160 | 281520 | 14 | 19 |
| 120 | SCT1.29 | 3981960 | 2269120 | 401640 | 10 | 6 |
| 121 | SCT1.30 | 5413400 | 1674520 | 464080 | 12 | 4 |
| 122 | SCT1.31 | 5741000 | 1524040 | 454360 | 13 | 3 |
| 123 | SCT1.32 | 4861600 | 2735160 | 336360 | 14 | 8 |
| 124 | SCT1.33 | 1469960 | 1789680 | 217200 | 7 | 8 |
| 125 | SCT1.34 | 500240 | 1936440 | 209480 | 2 | 9 |
| 126 | SCT1.45 | 128275720 | 120359200 | 9704920 | 13 | 12 |
| 127 | SCT1.46 | 127862600 | 129042360 | 12004320 | 11 | 11 |
| 128 | SCT1.47 | 127214360 | 127397280 | 14131400 | 9 | 9 |
| 129 | SCT1.48 | 128726800 | 131175440 | 8453880 | 15 | 16 |
| 100 | 7 | 126874080 | 133196840 | 8126320 | 16 | 16 |
| 237 | Palivzumab | 2696920 | 607840 | 2156440 | 1 | 0 |
| 246 | OKT4 | 8325560 | 105092760 | 4315400 | 2 | 24 |

¹RLU = relative light units

Antibodies were further screened for CD4 extracellular domain 3 (D3) binding using domain mapping. To perform domain mapping of CD4 antibodies, N-terminus Myc-tagged chimeric DNA constructs consisting of human CD4 domains swapped with mouse CD4 domains were generated in pTracer™-EF/V5-His A, B & C (Cat #V88701, Thermofisher). These chimeric DNA constructs along with human CD4, mouse CD4 and cynomolgus CD4 constructs (amino acid sequences provided in Table 10) were transfected into Expi293 cells (Cat: A14635, Thermofisher) according to manufacturer's protocol. One microgram total DNA was used per ml of transfection. After incubation with Epifectamine reagent at room temperature for 20 minutes, the DNA transfection complex was added to Expi293 cells at 3 million per ml. At 24 h post-transfection, CD4 transfected cells were harvested and stained with CD4 antibody panels measured by flow cytometry.

TABLE 10
Human/Mouse Chimeric Molecules Used for Domain Mapping and ELISA

| | SEQ ID | |
|---|---|---|
| Name | NO: | Amino acid sequence |
| Myc- | 1211 | EQKLISEEDLAAKKVVLGKKGDTVELTCTASQKKSIQFHWKNSNQ |
| hCD4 | | IKILGNQGSFLTKGPSKLNDRADSRRSLWDQGNFPLIIKNLKIED |
| | | SDTYICEVEDQKEEVQLLVFGLTANSDTHLLQGQSLTLTLESPPG |
| | | SSPSVQCRSPRGKNIQGGKTLSVSQLELQDSGTWTCTVLQNQKKV |
| | | EFKIDIVVLAFQKASSIVYKKEGEQVEFSFPLAFTVEKLTGSGEL |
| | | WWQAERASSSKSWITFDLKNKEVSVKRVTQDPKLQMGKKLPLHLT |
| | | LPQALPQYAGSGNLTLALEAKTGKLHQEVNLVVMRATQLQKNLTC |
| | | EVWGPTSPKLMLSLKLENKEAKVSKREKAVWVLNPEAGMWQCLLS |
| | | DSGQVLLESNIKVLPTWSTPVQPMALIVLGGVAGLLLFIGLGIFF |
| | | CVRCRHRRRQAERMSQIKRLLSEKKTCQCPHRFQKTCSPI |
| Myc- | 1212 | EQKLISEEDLAAKTLVLGKEGESAELPCESSQKKITVFTWKESDQ |
| mCD4 | | RKILGQHGKGVLIRGGSPSQFDRFDSKKGAWEKGSFPLIINKLKM |
| | | EDSQTYICELENRKEEVELWVFKVTFSPGTSLLQGQSLTLTLDSN |
| | | SKVSNPLTECKHKKGKVVSGSKVLSMSNLRVQDSDFWNCTVTLDQ |
| | | KKNWFGMTLSVLGFQSTAITAYKSEGESAEFSFPLNFAEENGWGE |
| | | LMWKAEKDSFFQPWISFSIKNKEVSVQKSTKDLKLQLKETLPLTL |
| | | KIPQVSLQFAGSGNLTLTLDKGTLHQEVNLVVMKVAQLNNTLTCE |
| | | VMGPTSPKMRLTLKQENQEARVSEEQKVVQVVAPETGLWQCLLSE |
| | | GDKVKMDSRIQVLSRGVNQTVFLACVLGGSFGFLGFLGLCILCCV |
| | | RCRHQQRQAARMSQIKRLLSEKKTCQCPHRMQKSHNLI |
| Myc- | 1213 | EQKLISEEDLAAKKVVLGKKGDTVELTCNASQKKNTQFHWKNSNQ |
| CCD4 | | IKILGIQGLFLTKGPSKLSDRADSRKSLWDQGCFSMIIKNLKIED |
| | | SDTYICEVENKKEEVELLVFGLTANSDTHLLEGQSLTLTLESPPG |
| | | SSPSVKCRSPGGKNIQGGRTISVPQLERQDSGTWTCTVSQDQKTV |
| | | EFKIDIVVLAFQKASSTVYKKEGEQVEFSFPLAFTLEKLTGSGEL |
| | | WWQAERASSSKSWITFDLKNKEVSVKRVTQDPKLQMGKKLPLHLT |
| | | LPQALPQYAGSGNLTLALEAKTGKLHQEVNLVVMRATQFQENLTC |
| | | EVWGPTSPKLTLSLKLENKGATVSKQAKAVWVLNPEAGMWQCLLS |
| | | DSGQVLLESNIKVVPTWPTPVQPMALIVLGGVAGLLLFTGLGIFF |
| | | CVRCRHRRRQAERMSQIKRLLSEKKTCQCPHRFQKTCSPI |
| Myc- | 1214 | EQKLISEEDLAAKKVVLGKKGDTVELTCTASQKKSIQFHWKNSNQ |
| hD1/ | | IKILGNQGSFLTKGPSKLNDRADSRRSLWDQGNFPLIIKNLKIED |
| mD2-4 | | SDTYICEVEDQKEEVQLLVFGLTFSPGTSLLQGQSLTLTLDSNSK |
| | | VSNPLTECKHKKGKVVSGSKVLSMSNLRVQDSDFWNCTVTLDQKK |
| | | NWFGMTLSVLGFQSTAITAYKSEGESAEFSFPLNFAEENGWGELM |
| | | WKAEKDSFFQPWISFSIKNKEVSVQKSTKDLKLQLKETLPLTLKI |
| | | PQVSLQFAGSGNLTLTLDKGTLHQEVNLVVMKVAQLNNTLTCEVM |
| | | GPTSPKMRLTLKQENQEARVSEEQKVVQVVAPETGLWQCLLSEGD |
| | | KVKMDSRIQVLSRGVNQTVFLACVLGGSFGFLGFLGLCILCCVRC |
| | | RHQQRQAARMSQIKRLLSEKKTCQCPHRMQKSHNLI |
| Myc- | 1215 | EQKLISEEDLAAKTLVLGKEGESAELPCESSQKKITVFTWKESDQ |
| hD2, | | RKILGQHGKGVLIRGGSPSQFDREDSKKGAWEKGSFPLIINKLKM |
| mD1, 3-4 | | EDSQTYICELENRKEEVELWVFKVTANSDTHLLQGQSLTLTLESP |
| | | PGSSPSVQCRSPRGKNIQGGKTLSVSQLELQDSGTWTCTVLQNQK |
| | | KVEFKIDIVVLAFQSTAITAYKSEGESAEFSFPLNFAEENGWGEL |
| | | MWKAEKDSFFQPWISFSIKNKEVSVQKSTKDLKLQLKETLPLTLK |
| | | IPQVSLQFAGSGNLTLTLDKGTLHQEVNLVVMKVAQLNNTLTCEV |
| | | MGPTSPKMRLTLKQENQEARVSEEQKVVQVVAPETGLWQCLLSEG |
| | | DKVKMDSRIQVLSRGVNQTVFLACVLGGSFGFLGFLGLCILCCVR |
| | | CRHQQRQAARMSQIKRLLSEKKTCQCPHRMQKSHNLI |
| Myc- | 1216 | EQKLISEEDLAAKTLVLGKEGESAELPCESSQKKITVFTWKESDQ |
| hD3, | | RKILGQHGKGVLIRGGSPSQFDREDSKKGAWEKGSFPLIINKLKM |
| mD1-2,4 | | EDSQTYICELENRKEEVELWVFKVTESPGTSLLQGQSLTLTLDSN |
| | | SKVSNPLTECKHKKGKVVSGSKVLSMSNLRVQDSDFWNCTVTLDQ |
| | | KKNWFGMTLSVLGFQKASSIVYKKEGEQVEFSFPLAFTVEKLTGS |
| | | GELWWQAERASSSKSWITFDLKNKEVSVKRVTQDPKLQMGKKLPL |
| | | HLTLPQALPQYAGSGNLTLALEAKTGKLHQEVNLVVMKVAQLNNT |
| | | LTCEVMGPTSPKMRLTLKQENQEARVSEEQKVVQVVAPETGLWQC |
| | | LLSEGDKVKMDSRIQVLSRGVNQTVFLACVLGGSFGFLGFLGLCI |
| | | LCCVRCRHQQRQAARMSQIKRLLSEKKTCQCPHRMQKSHNLI |
| Myc- | 1217 | EQKLISEEDLAAKTLVLGKEGESAELPCESSQKKITVFTWKESDQ |
| hD4, | | RKILGQHGKGVLIRGGSPSQFDRFDSKKGAWEKGSFPLIINKLKM |
| mD2-4 | | EDSQTYICELENRKEEVELWVFKVTFSPGTSLLQGQSLTLTLDSN |
| | | SKVSNPLTECKHKKGKVVSGSKVLSMSNLRVQDSDFWNCTVTLDQ |
| | | KKNWFGMTLSVLGFQSTAITAYKSEGESAEFSFPLNFAEENGWGE |
| | | LMWKAEKDSFFQPWISFSIKNKEVSVQKSTKDLKLQLKETLPLTL |
| | | KIPQVSLQFAGSGNLTLTLDKGTLHQEVNLVVMRATQLQKNLTCE |
| | | VWGPTSPKLMLSLKLENKEAKVSKREKAVWVLNPEAGMWQCLLSD |
| | | GDKVKMDSRIQVLSRGVNQTVFLACVLGGSFGFLGFLGLCILCCV |
| | | RCRHQQRQAARMSQIKRLLSEKKTCQCPHRMQKSHNLI |
| Myc- | 1218 | EQKLISEEDLAAKTLVLGKEGESAELPCESSQKKITVFTWKESDQ |
| mD1, | | RKILGQHGKGVLIRGGSPSQFDRFDSKKGAWEKGSFPLIINKLKM |
| hD2-4 | | EDSQTYICELENRKEEVELWVEKVTANSDTHLLQGQSLTLTLESP |
| | | PGSSPSVQCRSPRGKNIQGGKTLSVSQLELQDSGTWTCTVLQNQK |
| | | KVEFKIDIVVLAFQKASSIVYKKEGEQVEFSFPLAFTVEKLTGSG |
| | | ELWWQAERASSSKSWITFDLKNKEVSVKRVTQDPKLQMGKKLPLH |
| | | LTLPQALPQYAGSGNLTLALEAKTGKLHQEVNLVVMRATQLQKNL |
| | | TCEVWGPTSPKLMLSLKLENKEAKVSKREKAVWVLNPEAGMWQCL |
| | | LSDSGQVLLESNIKVLPTWSTPVQPMALIVLGGVAGLLLFIGLGI |
| | | FFCVRCRHRRRQAERMSQIKRLLSEKKTCQCPHRFQKTCSPI |
| Myc- | 1219 | EQKLISEEDLAAKTLVLGKEGESAELPCESSQKKITVFTWKESDQ |
| mD1-2, | | RKILGQHGKGVLIRGGSPSQFDRFDSKKGAWEKGSFPLIINKLKM |
| hD3-4 | | EDSQTYICELENRKEEVELWVEKVTESPGTSLLQGQSLTLTLDSN |
| | | SKVSNPLTECKHKKGKVVSGSKVLSMSNLRVQDSDFWNCTVTLDQ |
| | | KKNWFGMTLSVLGFQKASSIVYKKEGEQVEFSFPLAFTVEKLTGS |
| | | GELWWQAERASSSKSWITFDLKNKEVSVKRVTQDPKLQMGKKLPL |
| | | HLTLPQALPQYAGSGNLTLALEAKTGKLHQEVNLVVMRATQLQKN |
| | | LTCEVWGPTSPKLMLSLKLENKEAKVSKREKAVWVLNPEAGMWQC |
| | | LLSDGDKVKMDSRIQVLSRGVNQTVFLACVLGGSFGFLGFLGLCI |
| | | LCCVRCRHQQRQAARMSQIKRLLSEKKTCQCPHRMQKSHNLI |
| hCD4. | 1220 | KKVVLGKKGDTVELTCTASQKKSIQFHWKNSNQIKILGNQGSELT |
| Trp265 | | KGPSKLNDRADSRRSLWDQGNFPLIIKNLKIEDSDTYICEVEDQK |
| | | EEVQLLVFGLTANSDTHLLQGQSLTLTLESPPGSSPSVQCRSPRG |
| | | KNIQGGKTLSVSQLELQDSGTWTCTVLQNQKKVEFKIDIVVLAFQ |
| | | KASSIVYKKEGEQVEFSFPLAFTVEKLTGSGELWWQAERASSSKS |
| | | WITFDLKNKEVSVKWVTQDPKLQMGKKLPLHLTLPQALPQYAGSG |
| | | NLTLALEAKTGKLHQEVNLVVMRATQLQKNLTCEVWGPTSPKLML |
| | | SLKLENKEAKVSKREKAVWVLNPEAGMWQCLLSDSGQVLLESNIK |
| | | VLPTW |

Results of this analysis are shown in Table 11, where the binding mean fluorescence intensity (MFI) Ratio is defined as the MFI of CD4 transfected cells divided by the MFI of vector transfected cells. The results show a range of binding to human, cynomolgus and mouse CD4. None of the CD4-binding molecules identified through this campaign bound to mouse CD4. This is expected as mouse CD4 is more evolutionary divergent than human and cynomolgus CD4 are from one another. There may also have been negative selection against clones that recognize endogenous mouse CD4. The lack of cross-reactivity with mouse enabled accurate mapping of the CD4 domain recognized by each antibody. Most, though not all molecules bound both human CD4 and cynomolgus CD4. Consistent with previous findings that domain 2 (D2)-specific antibodies are more common than domain 3 (D3)-specific antibodies (Sattentau et al., J Exp Med (1989) 170:1319-1334), 18 antibodies were found to bind domain 2 or the junction between domain 2 and 3, and only 7 of the 108 clones originally isolated from the mouse immunization were specific for CD4 domain 3 (D3).

TABLE 11
Binding of CD4 Antibody Panel to Expi293 Cells Transfected with Chimeric CD4 Molecules

| | | | MFI Ratio (MFI CD4 transfected cells/MFI vector transfected cells | | | | | | | | |
|---|---|---|---|---|---|---|---|---|---|---|---|
| | Clone | Binding | hCD4 | mCD4 | CyCD4 | hD1 | hD2 | hD3 | hD4 | hD2-hD4 | hD3-hD4 |
| Molecule | name | domain | (1211) | (1212) | (1213) | (1214) | (1215) | (1216) | (1217) | (1218) | (1219) |
| 130 | 1.1 | D2 | 25.0 | 0.7 | 9.7 | 0.8 | 3.5 | 0.8 | 0.8 | 15.8 | 1.0 |
| 131 | 1.4 | D2 | 679 | 0.8 | 437.8 | 0.7 | 77.6 | 0.7 | 0.8 | 497.3 | 0.9 |
| 132 | 1.5 | D2 | 1083 | 0.5 | 481.7 | 0.5 | 11.7 | 0.6 | 0.6 | 390.3 | 0.8 |
| 133 | 2.9 | D2 | 10.6 | 0.8 | 29.6 | 0.8 | 231.3 | 1.1 | 1.1 | 8.1 | 0.9 |
| 137 | 2.10 | D2 | 13.7 | 1.2 | 327.5 | 1.2 | 9.7 | 1.1 | 1.4 | 12.3 | 1.1 |
| 138 | 2.11 | D2 | 117.6 | 0.6 | 299.1 | 0.9 | 10.8 | 0.9 | 1.1 | 9.4 | 0.8 |
| 143 | 2.12 | D2 | 41.6 | 0.6 | 5.1 | 0.7 | 18.9 | 0.6 | 0.9 | 20.4 | 0.8 |
| 149 | 2.13 | D2 | 47.2 | 0.8 | 56.4 | 1.0 | 116.6 | 1.0 | 1.2 | 43.1 | 0.9 |
| 150 | 2.14 | D2 | 74.7 | 0.8 | 341.9 | 0.9 | 122.8 | 1.0 | 1.1 | 23.2 | 0.8 |
| 153 | 2.15 | D2 | 12.6 | 0.7 | 155.3 | 0.9 | 39.0 | 0.9 | 0.9 | 8.6 | 0.8 |
| 157 | 1.6 | D2/D3 | 0.5 | 0.6 | 80.5 | 0.7 | 2.2 | 0.7 | 0.8 | 834.3 | 0.9 |
| 158 | 1.7 | D2/D3 | 2.6 | 0.5 | 196.0 | 0.6 | 0.7 | 0.7 | 0.9 | 267.1 | 0.9 |
| 159 | 1.8 | D2/D3 | 0.5 | 0.3 | 29.3 | 0.4 | 0.4 | 0.5 | 0.9 | 80.2 | 0.7 |
| 160 | 1.9 | D2/D3 | 2.2 | 0.3 | 128.6 | 0.4 | 0.5 | 0.6 | 0.7 | 309.2 | 0.7 |
| 161 | 1.10 | D2/D3 | 0.6 | 0.5 | 35.9 | 0.6 | 0.6 | 0.6 | 0.7 | 478.3 | 0.9 |
| 162 | 1.19 | D2/D3 | 221.1 | 0.6 | 177.5 | 0.7 | 0.6 | 0.8 | 0.8 | 134.1 | 1.0 |
| 163 | 2.2 | D2/D3 | 7.1 | 1.2 | 34.1 | 1.1 | 1.2 | 1.1 | 1.5 | 233.7 | 1.1 |
| 164 | 2.3 | D2/D3 | 3.0 | 0.6 | 8.8 | 0.8 | 1.1 | 1.2 | 2.0 | 13.6 | 1.0 |
| 165 | 1.20 | D3 | 329.2 | 0.5 | 299.0 | 0.6 | 0.6 | 8.0 | 0.8 | 280.2 | 3.8 |
| 168 | 1.21 | D3 | 876.8 | 0.7 | 521.4 | 0.8 | 0.7 | 10.3 | 0.9 | 383.9 | 4.8 |
| 172 | 1.22 | D3 | 876.8 | 0.7 | 521.4 | 0.8 | 0.7 | 10.3 | 0.9 | 383.9 | 4.8 |
| 176 | 1.23 | D3 | 939.8 | 0.6 | 574.0 | 0.7 | 0.8 | 11.4 | 0.8 | 149.5 | 6.7 |
| 183 | 1.24 | D3 | 781.8 | 0.7 | 586.4 | 0.7 | 0.8 | 12.4 | 0.8 | 589.5 | 5.0 |
| 187 | 2.1 | D3 | 8.5 | 0.7 | 105.4 | 1.0 | 0.8 | 3.5 | 1.0 | 47.9 | 3.2 |
| 188 | 2.8 | D3 | 7.6 | 0.9 | 33.4 | 1.6 | 3.5 | 8.9 | 1.2 | 22.0 | 3.9 |

CD4 is a well conserved target, but there are single-nucleotide polymorphisms (SNPs) in CD4 that are well described. Specifically, C868T encodes tryptophan at position 265 in place of arginine in domain 3. This SNP is highly prevalent among Africans (˜20%), predicted to change the tertiary structure of CD4, and associated with altered susceptibility to HIV infection (Oyugi et al, J Infect Dis (2009) 199(9):1327-34). This C868T mutation, encoding R256W amino acid substitution, also disrupts binding of the monoclonal antibody, OKT4 (Hodge et al., Human Immunology (1991) 30:99-104). For a therapy targeting HIV, it would be desirable to identify CD4 specific molecules that bind both polymorphisms, namely CD4 having either tryptophan and arginine at position 265 in CD4 domain 3. Human CD4 amino acid positions are with respect to SEQ ID NO: 1120; Uniprot P01730, herein.

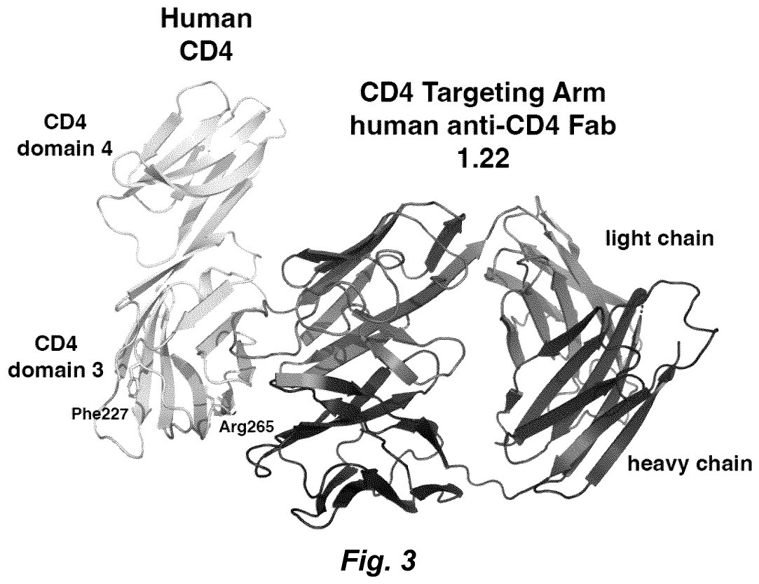

Enzyme-linked immunosorbent assay (ELISA) was used to determine if isolated CD4 domain 3 (D3) specific molecules were affected by the presence of a tryptophan at human CD4 position 265 (R265W). Human CD4 (hCD4) and hCD4 containing Trp265 (hCD4.Trp265) were coated at 2 μg/ml on Maxisorp plates (Thermo scientific, Cat #439454). Ten μg/ml of anti-CD4 domain 3 binders were applied and detected by goat anti-human IgG HRP (Jackson Immunotech, Cat #109-035-098). ELISA data is shown in FIG. 3. Consistent with previous literature, OKT4 bound hCD4 having arginine at CD4 position 265, but not hCD4.Trp265. All the domain 3-specific molecules described herein (VH/VL amino acid sequences provided in Table B) bound both hCD4 domain 3 (arginine at CD4 position 265) and the mutant hCD4 (tryptophan at CD4 position 265).

Molecules that paired CD4-specific binding domains that specifically bound epitopes within CD4 D1, D2, D2/D3 or D3 with IL-15 muteins were evaluated in the PBMC proliferation assay described in Example 1. For CD4-specific molecules, EC50 values ranged from 0.009 to 15.34 nM for CD4 activation and from 12 to >1000 nM for NK cell activation. The results are summarized in Table 12 (VH/VL amino acid sequences provided in Table B; IL-15v amino acid sequences provided in Tables D and E, CD4-targeted IL-15v amino acid sequences provided in Table G).

TABLE 12
EC50 Values for Proliferation of CD4+ Cells, CD8+ Cells and NK
Cells Induced by IL-15-Fc Variants with IgG1 or IgG4 Fc Domains

| | | | | | CD4 | CD8 | NK |
|---|---|---|---|---|---|---|---|
| | | CD4 | | | EC50 | EC50 | EC50 |
| Molecule | Fab Name | domain | IL-15 mutein | Fc | (nM) | (nM) | (nM) |
| 238 | Palivizumab | None | N4D/N65D | IgG1 | >1000 | >1000 | 140 |
| 245 | Ibalizumab | D1/D2 | N4D/N65D | IgG1 | 0.29 | >1000 | >1000 |
| 246 | OKT4 | D3 | N4D/N65D | IgG1 | 3.6 | >100 | >100 |
| 241 | M1 (mouse) | D1 | N4D/N65D | IgG1 | 0.13 | >1000 | >1000 |
| 240 | M1 (mouse) | D1 | N65D | IgG1 | 0.19 | >1000 | >1000 |
| 111 | 12 (humanized M1) | D1 | N65D | IgG1 | 0.044 | >1000 | 99 |
| 100 | 7 (humanized M1) | D1 | N65D | IgG1 | 0.056 | >100 | 54 |
| 131 | 1.4 | D2 | N65D | IgG1 | 0.24 | >100 | 70 |
| 132 | 1.5 | D2 | N65D | IgG1 | 0.027 | >100 | 49 |
| 133 | 2.9 | D2 | N65D | IgG1 | 0.064 | 490 | 34 |
| 137 | 2.10 | D2 | N65D | IgG1 | 0.13 | >100 | 56 |
| 138 | 2.11 | D2 | N65D | IgG1 | 0.10 | >100 | 94 |
| 143 | 2.12 | D2 | N65D | IgG1 | 0.12 | >100 | 62 |
| 149 | 2.13 | D2 | N65D | IgG1 | 0.017 | >100 | 59 |
| 150 | 2.14 | D2 | N65D | IgG1 | 0.016 | >100 | 34 |
| 153 | 2.15 | D2 | N65D | IgG1 | 0.014 | >100 | 83 |
| 157 | 1.6 | D2/D3 | N65D | IgG1 | 0.14 | 88 | 21 |
| 158 | 1.7 | D2/D3 | N65D | IgG1 | 0.034 | >100 | 43 |
| 159 | 1.8 | D2/D3 | N65D | IgG1 | 0.079 | >100 | >100 |
| 160 | 1.9 | D2/D3 | N65D | IgG1 | 0.029 | >100 | 40 |
| 161 | 1.10 | D2/D3 | N65D | IgG1 | 0.09 | >100 | 51 |
| 162 | 1.19 | D2/D3 | N65D | IgG1 | 15.34 | 480 | 42 |
| 163 | 2.2 | D2/D3 | N65D | IgG1 | 0.03 | >100 | 27 |
| 164 | 2.3 | D2/D3 | N65D | IgG1 | 0.042 | >100 | 41 |
| 165 | 1.20 | D3 | N65D | IgG1 | 0.98 | 780 | 71 |
| 168 | 1.21 | D3 | N65D | IgG1 | 0.28 | 150 | 28 |
| 172 | 1.22 | D3 | N65D | IgG1 | 0.14 | 780 | 30 |
| 176 | 1.23 | D3 | N65D | IgG1 | 0.25 | 590 | 30 |
| 183 | 1.24 | D3 | N65D | IgG1 | 0.68 | 160 | 12 |
| 187 | 2.1 | D3 | N65D | IgG1 | 0.71 | >100 | 61 |
| 188 | 2.8 | D3 | N65D | IgG1 | 0.034 | >100 | 44 |
| 109 | 10 (humanized M1) | D1 | N65D | IgG1 | 0.0090 | >1000 | 29 |
| | G54W LC | | | | | | |
| | T53/Y96Q | | | | | | |
| 247 | M1 (simianized | D1 | N65D | IgG1 | 0.081 | 950 | 100 |

To assess the developability of molecules with different CD4 binding domains, titers were measured from small scale expression in Expi293 cells five days after transfection. Clarified supernatant was purified using ProPlus PhyTips® on Agilent Bravo Liquid Handling platform. Bound molecules were eluted from ProPlus resin with 0.1 M Sodium Acetate, pH 3.5 and neutralized with 1 M Tris-HC1 pH 8.0 (V/V 1:10). Absorbance was quantified at A280 using Nanodrop after blanking with elution buffer. Concentration of each molecule recovered was determined by dividing the A280 reading with the respective extinction coefficient. Expression titer (mg/L) was calculated as follows: Concentration (mg/ml)×volume of eluate (ml)/volume of source*1000. Results are summarized in Table 13.

TABLE 13
Yields from Expression and Purification of CD4-targeted
IL-15v Molecules with Different CD4-Binding Domains

| | CD4 Binding | Anti-CD4 | IL15 | Titer |
|---|---|---|---|---|
| Molecule | domain | Clone name | Mutein | (mg/L) |
| 130 | D2 | 1.1 | N65D | 3.1 |
| 131 | D2 | 1.4 | N65D | 4.0 |
| 132 | D2 | 1.5 | N65D | 4.8 |
| 133 | D2 | 1.9 | N65D | 26 |
| 137 | D2 | 2.10 | N65D | 35 |
| 138 | D2 | 2.11 | N65D | 31 |
| 143 | D2 | 2.12 | N65D | 11 |
| 149 | D2 | 2.13 | N65D | 16 |
| 150 | D2 | 2.14 | N65D | 40 |
| 153 | D2 | 2.15 | N65D | 38 |
| 157 | D2/D3 | 1.6 | N65D | 1.6 |
| 158 | D2/D3 | 1.7 | N65D | 3.1 |
| 159 | D2/D3 | 1.8 | N65D | 5.8 |
| 160 | D2/D3 | 1.9 | N65D | 10.4 |
| 161 | D2/D3 | 1.10 | N65D | 7.7 |
| 162 | D2/D3 | 1.19 | N65D | 2.0 |
| 163 | D2/D3 | 2.2 | N65D | 35.6 |
| 164 | D2/D3 | 2.3 | N65D | 43 |
| 165 | D3 | 1.20 | N65D | 47 |
| 168 | D3 | 1.21 | N65D | 11 |
| 172 | D3 | 1.22 | N65D | 42 |
| 176 | D3 | 1.23 | N65D | 21 |
| 183 | D3 | 1.24 | N65D | 2.9 |
| 135 | D2 | 2.9 | N65D/I68A | 37 |
| 141 | D2 | 2.11 | N65D/I68A | 11 |
| | | LC.N50G | | |
| 146 | D2 | 2.12 | N65D/I68A | 20 |
| | | LC.N50G | | |
| 155 | D2 | 2.15 | N65D/I68A | 29 |
| 170 | D3 | 1.21 | N65D/I68A | 13 |
| 174 | D3 | 1.22 | N65D/I68A | 26 |
| 180 | D3 | 1.23 | N65D/I68A | 64 |
| | | LC.C36Y | | |
| 185 | D3 | 1.24 | N65D/I68A | 1.3 |

Additional modifications were made to improve developability. For clone 1.23, cysteine (C) was substituted to tyrosine (Y) at light chain (LC) position 36 to remove an unpaired cysteine. For clone 2.11 and 2.12, asparagine (N) was changed to glycine (G) at light chain position 50 to remove N-linked glycosylation and improve protein purity. Molecules with anti-CD4 clone 1.24 with N65D/168A or N65D IL-15 muteins had the lowest and 3rd lowest expression titers among all the clones screened.

### Example 3

### Structure of Anti-CD4 Fab Bound to Human CD4

We determined the structure of the complex formed by an anti-CD4 Fab binding human CD4 by cryoelectron microscopy. The purified complex of anti-CD4 Fab 1.22 and human CD4 ECD was mixed with equimolar amounts of an anti-human kappa light chain VHH (Thermo Fisher Scientific, cat #7103272500) to increase its molecular weight and facilitate structural determination by cryoelectron microscopy. A sample of 3.0 μl of the purified complex at 0.75 mg/ml was applied to glow-discharged (120 sec, 15 mA, PELCO easiGlow™, TED PELLA Inc.) 300 mesh UltrAuFoil Holey Gold R1.2/1.3 grids (Quantifoil), blotted for 2 seconds and vitrified with a Vitrobot Mark IV (Thermo Fisher Scientific) at 100% humidity and 10° C. A total of 8,239 cryoelectron micrographs, with a defocus range of 0.6 to 1.8 μm, were collected on a 300 kV Titan Krios transmission electron microscope (Thermo Fisher Scientific—FEI). A magnification of 165,000× was used corresponding to a pixel size of 0.729 Å. A Selectris-X energy filter (Thermo Fisher Scientific—FEI) filtered the images with an energy slit width of 10 eV and a Falcon 4i Direct Electron Detector (Thermo Fisher Scientific—FEI) in counting mode captured the images. A total electron dose of 52 electrons per Å2 was divided into 50 fractions. All cryo-EM image processing was carried out with the CryoSARC software suite. Movie fractions were aligned, and the contrast transfer function (CTF) corrected before automated particle picking. The initial steps of 2D classification were carried out with images binned 2-fold. The images belonging to well-defined 2D average classes were further processed by generating 3 ab initio maps which were refined by homogenous refinement. The set of images that led to the highest resolution map were extracted without binning and further refined with a non-uniform refinement protocol, optimizing per-particle scale and defocus, applying a noise-model for images and per-group CTF refinement. The initial individual protein models were generated with the Alpha-fold software and the final model of CD4/anti-CD4/anti-kappa light chain VHH complex was iteratively built and refined with the software suites Coot (Crystallographic Object-Oriented Toolkit; www2.mrc-lmb.cam.ac.uk/personal/pemsley/coot) and Phenix (phenix-online.org), respectively.

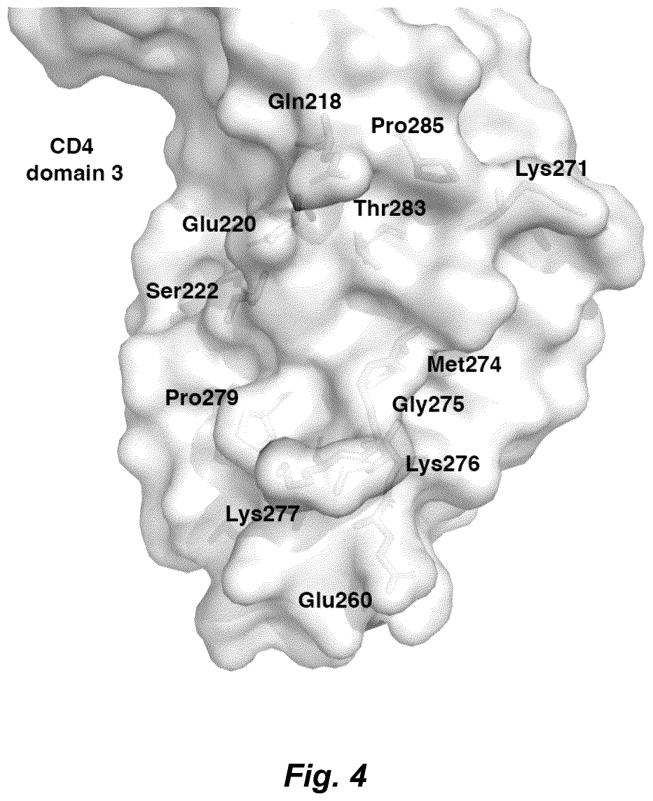

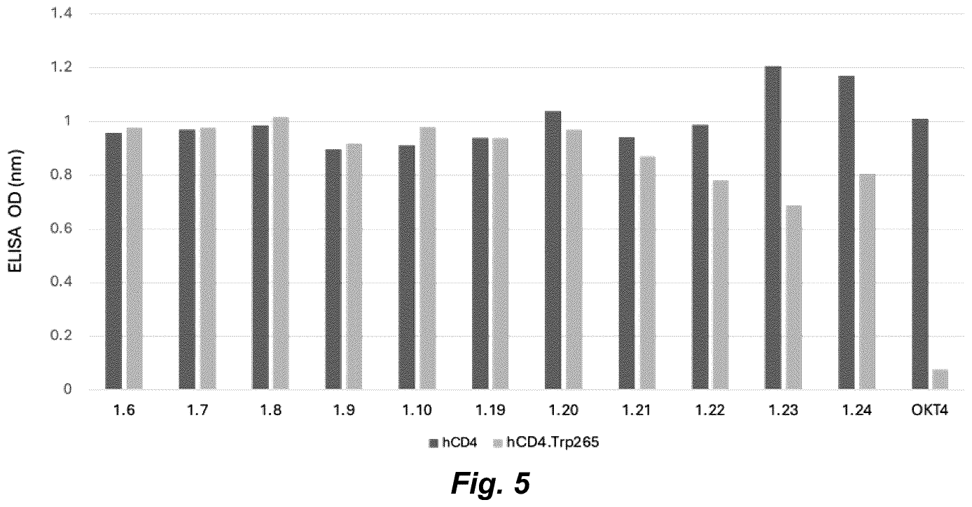

The 2.7 Å cryoelectron microscopy structure of the complex between anti-CD4-Fab SCT 1.22 and human CD4 confirms the binding of the human CD4 targeting arm to domain 3 (D3) of CD4 (FIG. 4). A map of the complex shows that all interactions between the anti-CD4 Fab and CD4 are within domain 3; moreover, most of the recognition contacts to CD4 occur with residues of the light chain of 1.22. A CD4 residue was considered to be in contact with the Fab if the van-der-Waals radius of any of its atoms was within 0.4 Å of an atom in the Fab as implemented in the analysis and visualization software program Chimera (rbvi.ucsf.edu/chimera/). The epitope recognized by the anti-CD4 Fab is exclusively in domain 3 of CD4 and is formed by residues 218, 220, 260, 271, 274-277, 279, 283 and 285 (FIG. 5). The structure of the complex confirms that the anti-CD4 Fab binding does not depend on recognition of known SNP positions Phe227 and Arg265.

### Example 4

### In Silico Identification of Amino Acid Residues Mediating Binding in IL15/IL15Rβγ Interface

To identify IL-15 residues for mutation, an in silico analysis was performed. The quaternary complex x-ray structure of the IL-15 receptor, comprising of the IL-15Rα, IL-2Rβ and IL-2Rγ subunits with its ligand (IL-15) bound, solved at a resolution of 2.35 Å (Ring, et al., Nature Immunology (2012) 13:1187-1197) was downloaded from the Protein Data Bank (PDB code: 4GS7 at rcsb.org) (Berman, et al., Nucleic Acids Res (2000) 28(1):235-42). The structure was prepared using the protein preparation tool in Maestro (Schrödinger.com, LLC, New York, NY, 2024), which involved assignment of correct atom type, bond order, protonation states and constrained minimization. The protein-protein interaction interfaces between IL-15/IL-15Rβ, IL-15/IL-15Rγ which has buried surface area (Stanton, et al., Anal. Chem. (1990) 62:2323-2329) of about 1250 Å2/about 1200 Å2 respectively were analyzed for residues with inter unit interactions which when substituted could reduce the affinity between the units.

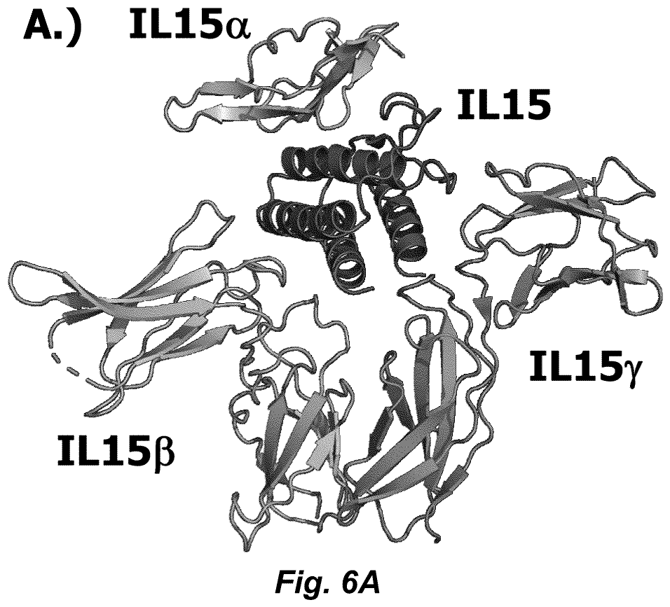

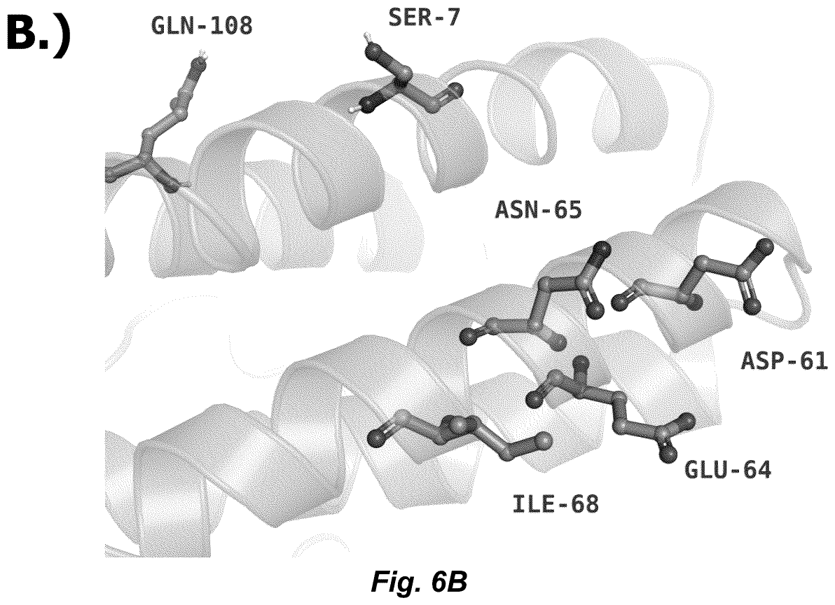

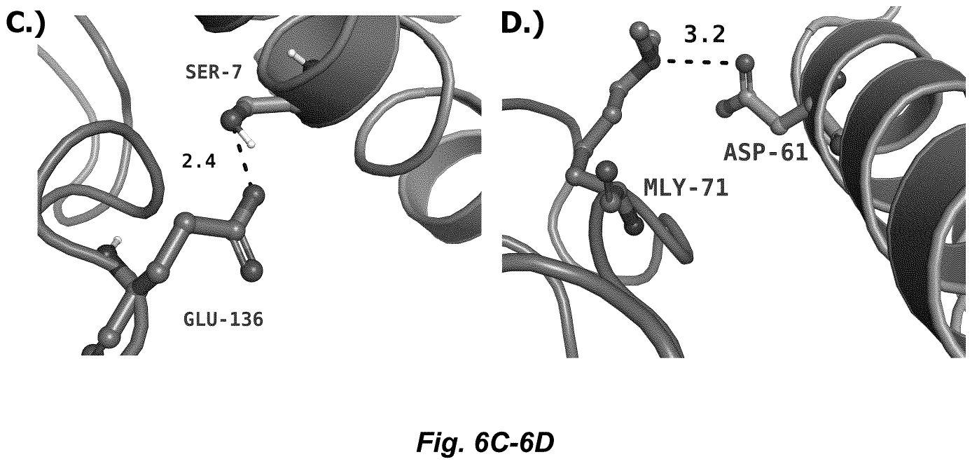

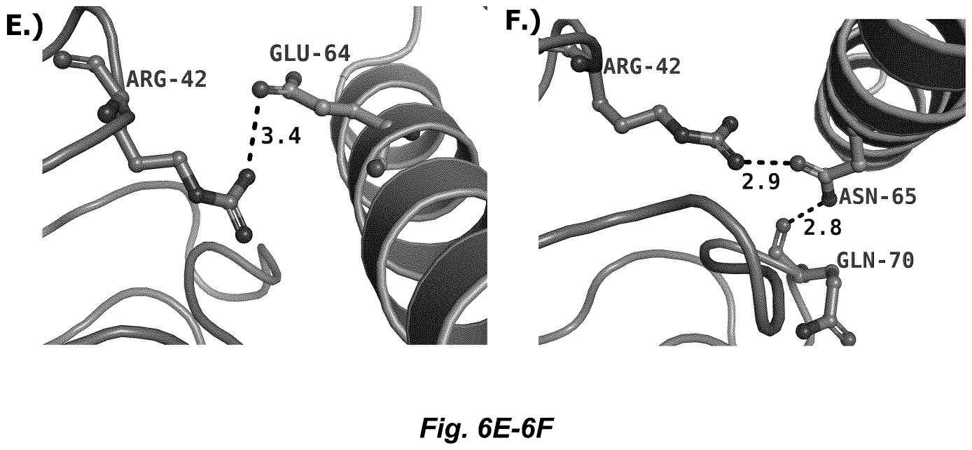

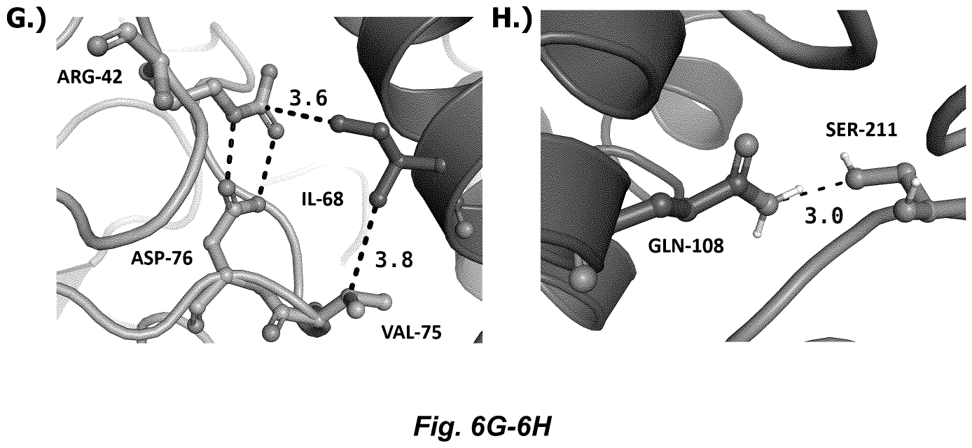

Based on this in silico analysis, a set of six residues were selected for further evaluation. The key inter and intra unit interactions observed for these residues are summarized in Table 14 and in FIG. 6. Single amino acid substitution studies were designed to evaluate their effect on the stability of the complex as well as protein expression.

TABLE 14
Summary of Each IL-15 Amino Acid Selected
to Evaluate Based on In Silico Analysis

| IL-15R | |
|---|---|
| Amino Acid | Interactions observed |
| Ser7 | Hydrogen bond with Glutamate 136 side chain of the |
| | IL-15Rβ subunit (a.k.a., IL-2Rβ subunit, IL2RB, |
| | IL15RB, CD122, NCBI Gene ID: 3560) |
| Asp61 | Salt bridge with methylated Lysine 71 (MLY) side |
| | chain of the IL-15Rβ subunit |
| Glu64 | Salt bridge with Arginine 42 side chain of the IL-15Rβ |
| | subunit |
| Asn65 | Hydrogen bond with Arginine 42 side chain and Glutamate |
| | 70 backbone of the IL-15Rβ subunit |
| Ile68 | Hydrophobic interaction with Valine 75 side chain as well |
| | as hydrophobic dehydroin generated from Arginine 42 and |
| | Aspartate 76 salt bridge of the IL-15Rβ subunit |
| Gln108 | Hydrogen bond with the Serine 211 side chain of the |
| | IL-15Rγ subunit (a.k.a., IL-2Rγ subunit, IL2RG, CD132, |
| | NCBI Gene ID: 3561) |

To validate in silico analysis identifying key residues required for IL-15 binding to IL-15R, various amino acid substitutions and combinations were generated. Molecules were assessed in the PBMC proliferation assay described in Example 1. The data demonstrated a range of potencies for CD4-targeted IL-15v fusion proteins: EC50s for CD4+ T cell activation (e.g., proliferation) ranged from 0.061 to >1000 nM and for NK cells ranged from 0.07 nM to greater than 1000 nM. Recombinant human IL-15 was 3-fold more potent in NK cells, as expected. CD4-targeted IL-15 with the wild-type IL-15 sequence (Molecule 256) was 28-fold more potent for CD4+ T cells than NK cells, showing that the targeting domain preferentially shifted activation to CD4+ T cells, as intended. All CD4-targeted IL-15v variants with Q108E (Molecules 250, 269, 272) lacked activity, showing this residue affects binding. D8G, N65D, and I68A amino acid substitutions (Molecules 263, 102, 254) reduced potency in NK cells to 160, 28 and 9.8 nM, respectively, while keeping CD4+ T cell potency within 2-fold of wild-type IL-15. Double and triple amino acid substitutions from wild-type IL-15 also reduced potency in NK cells, while generally preserving activity in stimulating proliferation of CD4+ T cells. Results are summarized in Table 15. In Table 15, molecules designated with Fab 8 (humanized M1) were made in the N-terminal fusion monovalent format (FIG. 1C). Molecules in Table 16 that are designated bivalent were made in the C-terminal bivalent format (FIG. 1F). Fab amino acid sequences are in Table B, IL-15v amino acid sequences are in Tables D and E; CD4-targeted IL-15v amino acid sequences are in Table G.

TABLE 15
EC50 Values for Proliferation of CD4+ Cells, CD8+ Cells and NK
Cells Induced by IL-15-Fc Variants with IgG1 or IgG4 Fc Domains

| Molecule | Fab Name | CD4 domain | IL-15 mutein | Fc | CD4 EC50 (nM) | CD8 EC50 (nM)NK | EC50 (nM) |
|---|---|---|---|---|---|---|---|
| IL-15 | None | D1-D4 | WT | NA | 0.193 | 0.014 | 0.07 |
| 256 | 8 (humanized M1) | D1 | WT | IgG1 | 0.025 | 5.2 | 0.7 |
| 106 | 8 (humanized M1) | D1 | N65D/I68A | IgG1 | 0.10 | >1000 | 810 |
| 110 | 8 (humanized M1) | D1 | S7G/N65D | IgG1 | 0.043 | 760 | 200 |
| 248 | 8 (humanized M1) | D1 | D8G/N65D | IgG1 | ND | ND | ND |
| 249 | 8 (humanized M1) | D1 | N65A/I68A | IgG1 | 0.17 | >1000 | 900 |
| 250 | 8 (humanized M1) | D1 | Q108E | IgG1 | ND | ND | ND |
| 251 | 8 (humanized M1) | D1 | D61N/N65D/I68A | IgG1 | >1000 | >1000 | >1000 |
| 105 | 8 (humanized M1) | D1 | D30N/E64Q/N65D | IgG1 | 0.040 | >3000 | >3000 |
| 252 | 8 (humanized M1) | D1 | N4G/N65D | IgG1 | 0.010 | >100 | >100 |
| 253 | 8 (humanized M1) | D1 | N4D/N65D | IgG1 | 0.028 | >1000 | 92 |
| 254 | 8 (humanized M1) | D1 | I68A | IgG1 | 0.011 | >1000 | 9.8 |
| 255 | 8 (humanized M1) | D1 | D30N/N65D | IgG1 | 0.026 | >1000 | 120 |
| 104 | 8 (humanized M1) | D1 | E64Q/N65D | IgG1 | 0.079 | >1000 | >1000 |
| 103 | 8 (humanized M1) | D1 | D61N/N65D | IgG1 | 0.065 | >100 | >100 |
| 102 | 8 (humanized M1) | D1 | N65D | IgG1 | 0.021 | >100 | 28 |
| 257 | 8 (humanized M1) | D1 | S7G/D8G/N65D | IgG1 | >100 | >100 | >100 |
| 258 | 8 (humanized M1) | D1 | N4G/D8G/N65D | IgG1 | >100 | >100 | >100 |
| 259 | 8 (humanized M1) | D1 | N4G/S7G/N65D | IgG1 | 0.14 | >100 | >100 |
| 260 | 8 (humanized M1) | D1 | E64A/N65D | IgG1 | 0.040 | >1000 | 170 |
| 261 | 8 (humanized M1) | D1 | D61A/N65D | IgG1 | 0.10 | >1000 | 670 |
| 262 | 8 (humanized M1) | D1 | E64Q/N65D/I68A | IgG1 | >1000 | >1000 | >1000 |
| 222 | 1.22 bivalent | D3 | D8G | IgG4 | 0.035 | >1000 | 160 |
| 223 | 1.22 bivalent | D3 | N65D/I68A | IgG4 | 0.029 | >300 | 130 |
| 224 | 1.22 bivalent | D3 | N65D/I68A/S7G | IgG4 | 0.061 | >1000 | 450 |
| 225 | 1.22 bivalent | D3 | N65A/I68A/S7G | IgG4 | 0.097 | >300 | >300 |
| 226 | 1.22 bivalent | D3 | D8G/N65D | IgG4 | 0.082 | >1000 | >1000 |
| 227 | 1.22 bivalent | D3 | N65A/I68A | IgG4 | 0.025 | >300 | 130 |
| 228 | 1.22 bivalent | D3 | Q108E | IgG4 | >300 | 250 | 8.6 |
| 229 | 1.22 bivalent | D3 | D61N/N65D/I68A | IgG4 | 0.11 | >1000 | >1000 |
| 230 | 1.22 bivalent | D3 | D8G/E64Q | IgG4 | 0.19 | >1000 | >1000 |
| 231 | 1.22 bivalent | D3 | E64Q/Q108E | IgG4 | >300 | >300 | >300 |

### Example 5

### Design of Glycan and Deamidation Mitigation Variants

There are three N-linked glycosylation sites (N71, N79, N112) and one deamidation site (N77) in wild-type human IL-15. N-linked glycosylation patterns can exhibit heterogeneity, both in terms of prevalence as well as actual glycoform species. Although glycosylation can have favorable effects on proteins such as increased solubility and stability, this increased heterogeneity may alter the intended functional properties of the biologic. Consequently, the removal of glycosylation sites, via amino acid substitutions, is desired to generate homogenous, aglycosylated proteins. Similarly, certain solvent-exposed asparagine residues may be deamidated by the solvent, resulting in heterogenous and potentially functionally altered molecules.

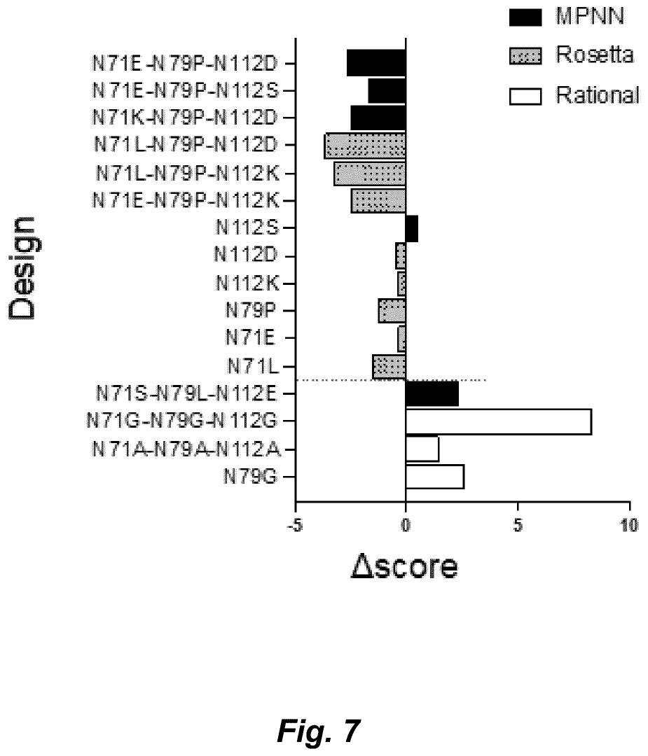

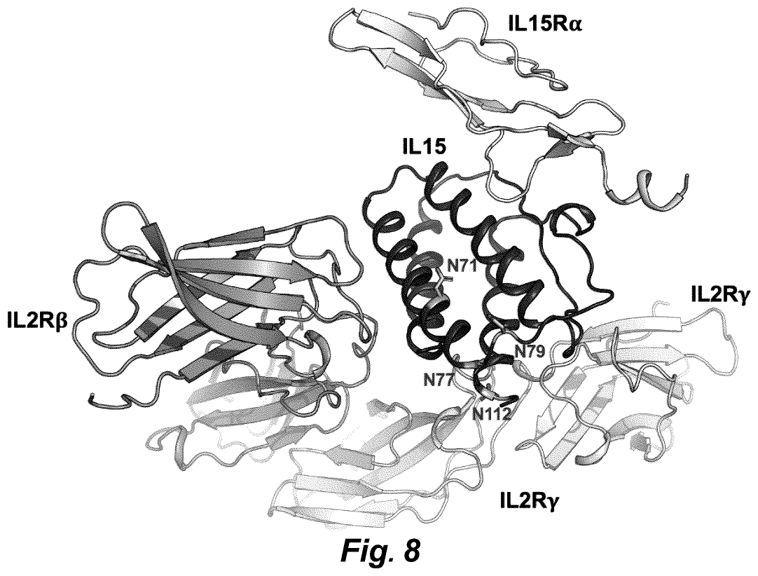

In this example, computational protein design tools (AlphaFold (alphafold.ebi.ac.uk/), Rosetta (rosettacommons.org), ProteinMPNN (Dauparas, et al., Science (2022) 378(6615):49-56)) were applied to counteract such stability and solubility losses by predicting protein variants which improve the relative stability of IL-15 variants relative to wild-type (WT) IL-15, and remove glycosylation and/or deamidation sites in IL-15 while minimizing structural changes to reduce the risk of altered protein function. Models of apo-IL-15 and IL-15-receptor complex structures were generated using AlphaFold Multimer (Evans, et al., bioRxiv 2021.10.04.463034 (2022); see also, Abramson, et al., Nature. (2024) 630(8016):493-500). In both cases, the highest confidence models were relaxed with constraints using Rosetta (Alford, et al., J Chem Theory Comput (2017) 13, 3031-3048) until scores converged to a minimum. The lowest scoring models were used as input for downstream design protocols where the IL-15 design positions were N71, N77, N79, and N112 either individually or in combination. Rosetta flexible backbone design protocol and ProteinMPNN (Dauparas, et al., supra) protocols were used to predict the most favorable residues at each design position. Rosetta is a physics-based tool which calculates the energetic contribution of intra- and inter-residue interactions to predict the most stable residue at a given position. The output of the Rosetta protocol is a AE score which describes the relative predicted change in stability of a design, where a negative value signifies a more stable molecule relative to WT. ProteinMPNN is a machine-learning-based protein structure design tool which predicts the identity of a given residue given its local three-dimensional structural context. The probabilistic output of ProteinMPNN can be used to infer the bias for a given residue at each of the design positions. Several of the most promising variants at each design position were chosen for experimental validation. Designs which were predicted to be favorable by both Rosetta and ProteinMPNN were prioritized (FIGS. 7 and 8).

To validate in silico predictions for glycosylation and deamidation variants, several variant combinations were tested in the CD4-Targeted IL-15 bispecific format. Molecules were screened for potency in a KHYG-1 cell line. This NK cell line is dependent on IL-2 or IL-15 for growth and does not endogenously express CD4, so a derivative cell line was engineered to express high levels of human CD4 (KHYG-1 hCD4).

KHYG-1 hCD4 Proliferation Assay. The potency of CD4-targeted IL-15v molecules was evaluated by measuring the proliferation of KHYG-1 hCD4 cells. Briefly, cells were washed and maintained in growth media devoid of IL-12/IL15 overnight. Cells were subsequently plated at a density of 5,000 cells per well and incubated for 24 hours with a dose titration of the CD4-targeted IL15 molecule. The addition of CellTiter-Glo reagent (Promega, Cat #G7572) resulted in cell lysis and generation of luminescent signals via luciferase reaction. The luminescent signal is proportional to the amount of ATP present, indicating cell proliferation. EC50s were calculated based on the percentage of the luminescent signal generated by treating KHYG-1 cells with the CD4-targeted IL15 molecules.

The results summarized in Table 16 show that all tested IL-15 variants incorporated into CD4-targeted IL-15v molecules retain activity and support proliferation of KHYG-1 hCD4 cells. These molecules were each made using the format shown in FIG. 1C. A representative subset of these molecules was selected for evaluation in the PBMC Proliferation Assay described in Example 1 to measure potency and selectivity in isolated PBMCs in vitro. Amino acid sequences of IL-15v are provided in Tables D and E; amino acid sequences of CD4-targeted IL-15v provided in Table G.

TABLE 16
In Vitro Potency of Glycan Variants in the KHYG-hCD4 Proliferation Assay

| | Fab | CD4 | | | KHYG-CD4 |
|---|---|---|---|---|---|
| Molecule | Name | domain | IL-15 mutein | Fc | EC50 (nM) |
| 173 | 1.22 | D3 | S7G/N65D | IgG1 (P331S) | 0.459 |
| 189 | 1.22 | D3 | S7G/N65D/N71L/N77L/N79P/N112K | IgG1 (G237A) | 0.180 |
| 191 | 1.22 | D3 | S7G/N65D/N71L/N77L/N79P/N112K | IgG1 (P331S) | 0.054 |
| 192 | 1.22 | D3 | S7G/N65D/N71L | IgG1 (P331S) | 0.308 |
| 193 | 1.22 | D3 | S7G/N65D/N71E | IgG1 (P331S) | 0.325 |
| 194 | 1.22 | D3 | S7G/N65D/N77L | IgG1 (P331S) | 0.512 |
| 195 | 1.22 | D3 | S7G/N65D/N79P | IgG1 (P331S) | 0.359 |
| 196 | 1.22 | D3 | S7G/N65D/N112K | IgG1 (P331S) | 0.157 |
| 197 | 1.22 | D3 | S7G/N65D/N112D | IgG1 (P331S) | 0.243 |
| 198 | 1.22 | D3 | S7G/N65D/N112S | IgG1 (P331S) | 0.074 |
| 199 | 1.22 | D3 | S7G/N65D/N71L/N79P | IgG1 (P331S) | 0.046 |
| 200 | 1.22 | D3 | S7G/N65D/N71L/N112D | IgG1 (P331S) | 0.052 |
| 201 | 1.22 | D3 | S7G/N65D/N79P/N112D | IgG1 (P331S) | 0.027 |
| 202 | 1.22 | D3 | S7G/N65D/N71Q/N79Q/N112Q | IgG1 (P331S) | 0.086 |
| 203 | 1.22 | D3 | S7G/N65D/N71A/N79A/N112A | IgG1 (P331S) | 0.055 |
| 204 | 1.22 | D3 | S7G/N65D/N71G/N79G/N112G | IgG1 (P331S) | 0.067 |
| 205 | 1.22 | D3 | S7G/N65D/N71S/N79L/N112E | IgG1 (P331S) | 0.144 |
| 206 | 1.22 | D3 | S7G/N65D/N71E/N79P/N112K | IgG1 (P331S) | 0.097 |
| 207 | 1.22 | D3 | S7G/N65D/N71L/N79P/N112K | IgG1 (P331S) | 0.150 |
| 208 | 1.22 | D3 | S7G/N65D/N71L/N79P/N112D | IgG1 (P331S) | 0.247 |
| 209 | 1.22 | D3 | S7G/N65D/N71K/N79P/N112D | IgG1 (P331S) | 0.365 |
| 210 | 1.22 | D3 | S7G/N65D/N71E/N79P/N112S | IgG1 (P331S) | 0.053 |
| 211 | 1.22 | D3 | S7G/N65D/N71E/N79P/N112D | IgG1 (P331S) | 0.090 |
| 212 | 1.22 | D3 | S7G/N65D/N79P | IgG1 (P331S) | 0.127 |
| 213 | 1.22 | D3 | S7G/N65D/N112K | IgG1 (P331S) | 0.066 |
| 214 | 1.22 | D3 | S7G/N65D/N112D | IgG1 (P331S) | 0.067 |
| 215 | 1.22 | D3 | S7G/N65D/N71S/N77L/N79L/N112E | IgG1 (P331S) | 0.118 |
| 216 | 1.22 | D3 | S7G/N65D/N71E/N77L/N79P/N112K | IgG1 (P331S) | 0.133 |
| 217 | 1.22 | D3 | S7G/N65D/N71E/N77L/N79P/N112S | IgG1 (P331S) | 0.054 |
| 218 | 1.22 | D3 | S7G/N65D/N71E/N77L/N79P/N112D | IgG1 (P331S) | 0.136 |
| 220 | 1.22 | D3 | S7G/N65D/N79G | IgG1 (P331S) | 0.380 |
| 278 | 1.22 | D3 | N65D/I68A/N71L/N77L/N79P/N112K | IgG1 (P331S) | 0.17 |

The results in Table 17 show that the designed glycan and deamidation variants maintain potency in human CD4+ T cells, with EC50s ranging from 0.081 to 1.2 nM. These molecules also had high selectivity, with EC50s ranging from 36 to >1000 nM in NK cells. Several of these variants (e.g. Molecules 189, 191, 197, 208, 209, 278) showed CD4 potency that was similar to the parent molecule (173) but with even lower activity in NK cells, indicating selectivity could be improved by these substitutions.

TABLE 17
EC50 Values for Proliferation of CD4+ T cells, CD8+ T cells
and NK cells Induced by IL-15-Fc Glycan and Deamidation Variants

| | | | | | CD4 | CD8 | NK |
|---|---|---|---|---|---|---|---|
| | Fab | CD4 | | | EC50 | EC50 | EC50 |
| Molecule | Name | domain | IL-15 mutein | Fc | (nM) | (nM) | (nM) |
| 173 | 1.22 | D3 | S7G/N65D | IgG1 | 0.70 | >1000 | 595 |
| | | | | (P331S) | | | |
| 189 | 1.22 | D3 | S7G/N65D/N71L/N77L/N79P/N112K | IgG1 | 0.35 | >1000 | >1000 |
| | | | | (G237A) | | | |
| 191 | 1.22 | D3 | S7G/N65D/N71L/N77L/N79P/N112K | IgG1 | 0.23 | 31 | >1000 |
| | | | | (P331S) | | | |
| 192 | 1.22 | D3 | S7G/N65D/N71L | IgG1 | 0.44 | >1000 | 410 |
| | | | | (P331S) | | | |
| 195 | 1.22 | D3 | S7G/N65D/N79P | IgG1 | 0.47 | 1000 | 280 |
| | | | | (P331S) | | | |
| 197 | 1.22 | D3 | S7G/N65D/N112D | IgG1 | 1.1 | >1000 | >1000 |
| | | | | (P331S) | | | |
| 199 | 1.22 | D3 | S7G/N65D/N71L/N79P | IgG1 | 0.36 | >1000 | 410 |
| | | | | (P331S) | | | |
| 200 | 1.22 | D3 | S7G/N65D/N71L/N112D | IgG1 | 1.2 | >1000 | >1000 |
| | | | | (P331S) | | | |
| 201 | 1.22 | D3 | S7G/N65D/N79P/N112D | IgG1 | 0.62 | >1000 | 980 |
| | | | | (P331S) | | | |
| 202 | 1.22 | D3 | S7G/N65D/N71Q/N79Q/N112Q | IgG1 | 0.39 | >1000 | 130 |
| | | | | (P331S) | | | |
| 208 | 1.22 | D3 | S7G/N65D/N71L/N79P/N112D | IgG1 | 0.95 | >1000 | >1000 |
| | | | | (P331S) | | | |
| 209 | 1.22 | D3 | S7G/N65D/N71K/N79P/N112D | IgG1 | 1.18 | >1000 | >1000 |
| | | | | (P331S) | | | |
| 216 | 1.22 | D3 | S7G/N65D/N71E/N77L/N79P/N112K | IgG1 | 0.27 | >1000 | 350 |
| | | | | (P331S) | | | |
| 217 | 1.22 | D3 | S7G/N65D/N71E/N77L/N79P/N112S | IgG1 | 0.081 | >1000 | 36 |
| | | | | (P331S) | | | |
| 218 | 1.22 | D3 | S7G/N65D/N71E/N77L/N79P/N112D | IgG1 | 0.41 | >600 | 590 |
| | | | | (P331S) | | | |
| 278 | 1.22 | D3 | N65D/I68A/N71L/N77L/N79P/N112K | IgG1 | 0.84 | >1000 | >1000 |
| | | | | (P331S) | | | |

### Example 6

### HIV Activation by CD4-Targeted IL-15

In this example, we assessed the ability of CD4-targeted IL-15v to activate HIV PBMCs isolated from ART-suppressed people with HIV (PWH).

PBMC were isolated from ART-suppressed PWH (plasma HIV RNA levels less than (<) 50 copies/ml for at least 1 year) were resuspended in RPMI 1640 media with 10% fetal bovine serum, penicillin-streptomycin and antivirals (100 nM elvitegravir and 100 nM efavirenz) at 3 million cells/ml. Cells were seeded at 15 million cells per well in a 6-well plate and treated with dimethylsulfoxide (DMSO), 1 nM recombinant human IL-15, 1 nM CD4-targeted IL-15v (Molecule 101) or PMA (50 ng/ml) and ionomycin (500 ng/ml). Cells were incubated for 4 days at 37° C. Cells were centrifuged and supernatants were collected. HIV RNA levels were quantified using the COBAS®AmpliPrep/COBAS® TaqMan® HIV-1 Test, v2.0 (Roche) according to manufacturer's instructions. The geometric mean of six replicates was calculated per condition for each donor. PBMCs from 17 donors were tested.

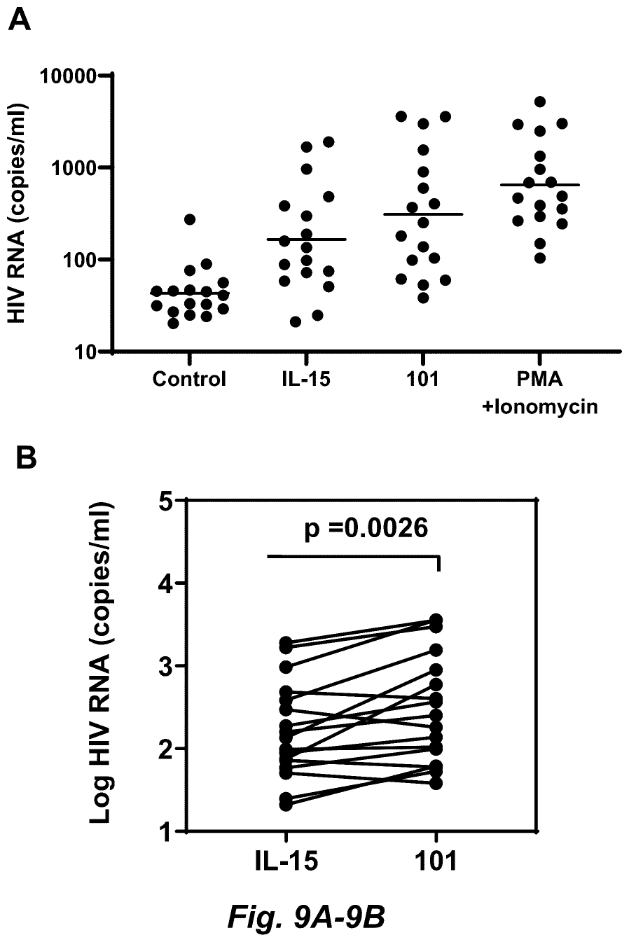

Table 18 and FIG. 9 show that CD4-targeted IL-15v activates HIV RNA production relative to the DMSO control. Unexpectedly, HIV activation was stronger with CD4-targeted IL-15v than with recombinant human IL-15 (FIG. 9B). As expected, treatment with the strong mitogenic stimuli, PMA and ionomycin, induced the highest levels of HIV RNA (FIG. 9A).

TABLE 18
HIV expression after treatment of PBMC
from PWH with CD4-targeted IL-15

| | | HIV RNA |
|---|---|---|
| | HIV RNA | (fold change |
| Condition | (copies/ml) | from DMSO) |
| DMSO | 43 | 1.0 |
| IL-15 | 170 | 3.8 |
| CD4-targeted IL-15v | 310 | 7.2 |
| (Molecule 101) | | |
| PMA/Ionomycin | 650 | 15 |

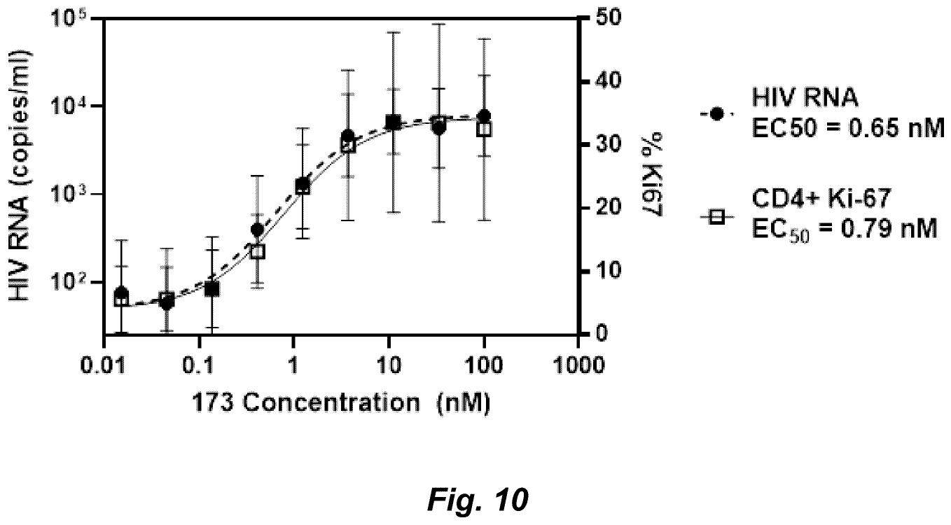

To determine if doses that activate CD4+ T cells also activate HIV, the method described above was used, PBMCs were isolated from PWH (N=6) that were known to have a strong response to CD4-Targeted IL-15. Cells were treated with various concentrations of CD4-targeted IL-15v (Molecule 173) in RPMI 1640 media with 10% fetal bovine serum, penicillin-streptomycin and antivirals (100 nM elvitegravir and 100 nM efavirenz) at 3 million cells/ml. Cells were incubated for 7 days at 37° C. Supernatants were collected and analyzed using COBAS® AmpliPrep/COBAS® TaqMan® HIV-1 Test, v2.0 to measure HIV RNA. Cells were also stained for Ki67 and analyzed by flow cytometry described in the PBMC proliferation assay in Example 1. Both HIV RNA and proliferation results were analyzed on GraphPad Prism using four parameter variable slope dose response curves to determine the half maximal effective concentration (EC50). Dose dependent induction of HIV RNA and Ki67 was observed and the results are depicted in FIG. 10. The EC50 for HIV RNA was 0.65 nM and for Ki67 was 0.79 nM. A strong correlation was observed between activation and HIV RNA induction in this assay.

To compare HIV reactivation by different CD4-targeted IL-15v molecules, PBMCs were isolated from ART-suppressed PWH (N=4-15) resuspended at 3 million cells/ml in RPMI 1640 media with 10% fetal bovine serum, penicillin-streptomycin and antivirals (100 nM elvitegravir and 100 nM efavirenz). Cells were treated with each CD4-targeted IL-15v molecule at a concentration that was 30-times higher than the EC50, as determined by CD4+ T cell proliferation in PBMCs. Cells were incubated for 7 days at 37° C. Supernatants were collected and analyzed using COBAS® AmpliPrep/COBAS® TaqMan® HIV-1 Test, v2.0 to measure HIV RNA.

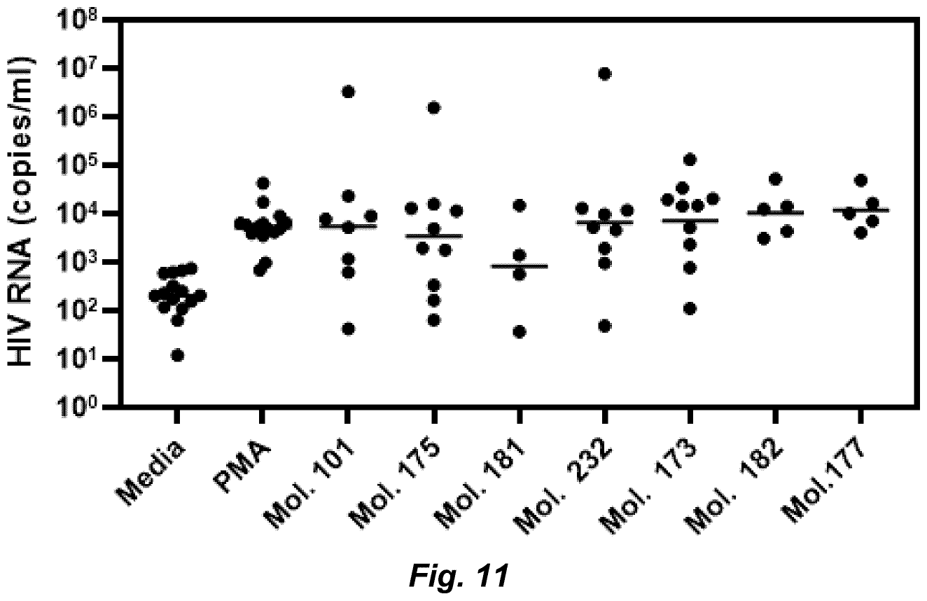

Table 19 and FIG. 11 show that all CD4-targeted IL-15v molecules tested induced activation of HIV RNA in PBMC from PWH. This induction ranged from 4-fold over background to 59-fold. In this set of donors, most CD4-targeted IL-15v molecules induced HIV activation at levels that were comparable to those induced by PMA and ionomycin, the mitogenic control stimulus. The data are consistent with the conclusion that the CD4-targeted IL-15v molecules induce strong activation of HIV expression in PBMC isolated from ART-suppressed PWH.

TABLE 19
HIV Activation by CD4-Targeted IL-15

| | | | Number |
|---|---|---|---|
| | Geomean | Fold | of donors |
| Media | 200 | 1 | 15 |
| PMA | 5100 | 25 | 15 |
| Molecule 101 | 5500 | 27 | 8 |
| Molecule 175 | 3400 | 17 | 10 |
| Molecule 181 | 810 | 4 | 4 |
| Molecule 232 | 6600 | 33 | 9 |
| Molecule 173 | 7200 | 36 | 10 |
| Molecule 182 | 10400 | 52 | 5 |
| Molecule 177 | 12000 | 59 | 5 |

### Example 7

### CD4-Targeted IL-15v Enhances HIV Activation by TLR8 and NOD2 Agonists

In this example, we evaluated multiple pattern recognition receptor agonists as potential strategies for further enhancing HIV activation induced by CD4-targeted IL-15v molecules. Several studies have indicated that pattern recognition receptors can activate HIV expression (Takahama et al., Front Cell Infect Microb (2020) 10:216).

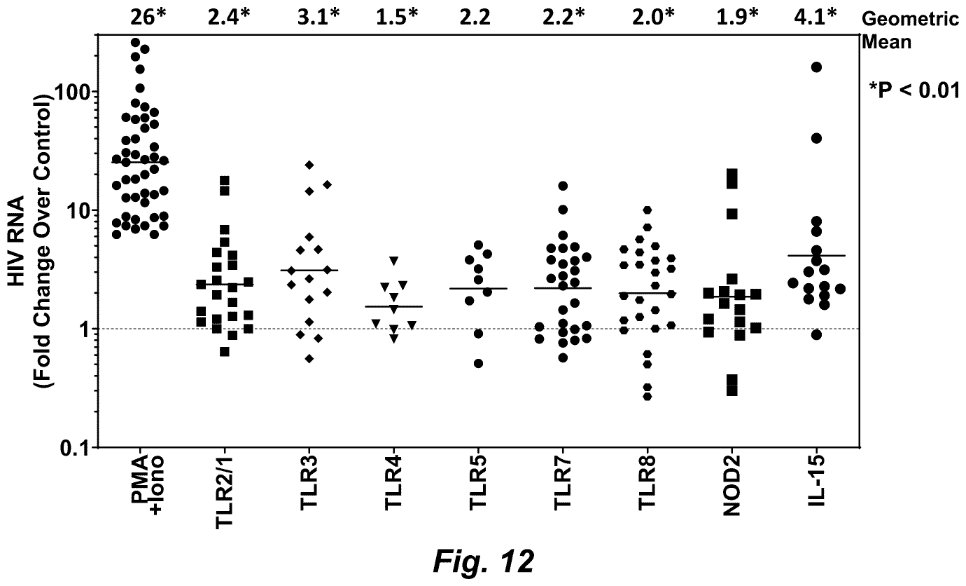

To identify pattern recognition receptor agonists that could enhance HIV expression, a panel of agonists was tested for the ability to activate HIV in PBMC from ART-suppressed PWH. Isolated PBMC cultured in RPMI 1640 media with 10% fetal bovine serum and penicillin-streptomycin at 3 million cells/ml in the presence of antivirals (100 nM elvitegravir and 100 nM efavirenz) and treated with 0.1 μg/ml Pam3CSK4 (toll-like receptor (TLR) 2/1 agonist), 0.1 μg/ml high molecular weight poly (I: C) (TLR3 agonist), 1 μg/ml monophosphoryl lipid A (TLR4 agonist), 0.5 μg/ml recombinant Flagellin, S. typhimuium (TLR5 agonist), 100 nM GS-9620 (vesatolimod; TLR7 agonist), 300 nM GS-9688 (selgantolimod; TLR8 agonist), 0.1 μM romurtide (Nucleotide binding Oligomerization Domain-2, NOD2) or 1 nM recombinant wild-type human IL-15 (SEQ ID NO: 740; Table D). Cells were seeded at 15 million cells per well in a 6-well plate with 12 replicates per condition and incubated at 37° C. for 4 days. Cells were centrifuged, supernatants were collected, and HIV RNA levels were quantified using the COBAS®AmpliPrep/COBAS® TaqMan® HIV-1 Test, v2.0 (Roche) according to manufacturer's instructions. Each treatment condition was normalized to the vehicle control (DMSO). Results of this analysis are shown in FIG. 12. Agonists for TLR2/1, TLR3, TLR7, TLR8 and NOD2 significantly induced HIV expression at least 2-fold.

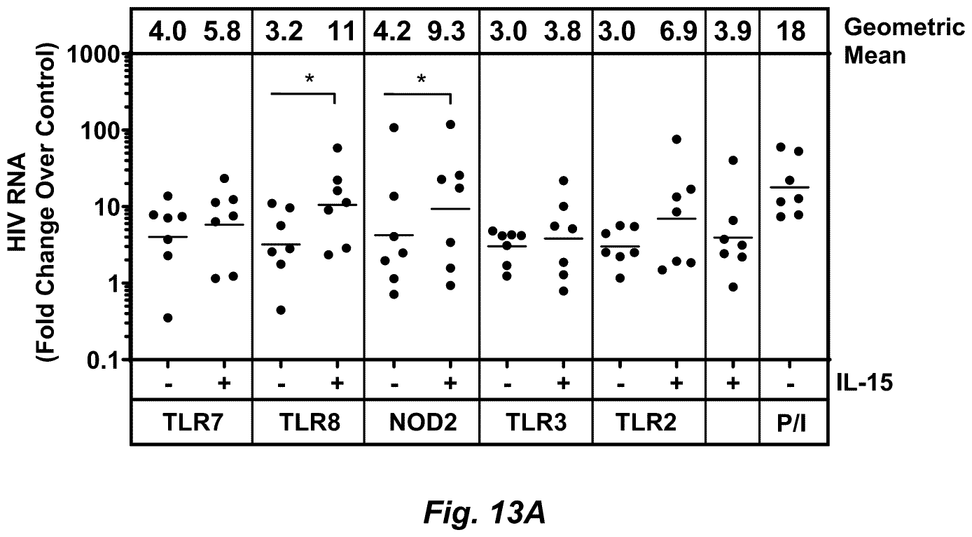

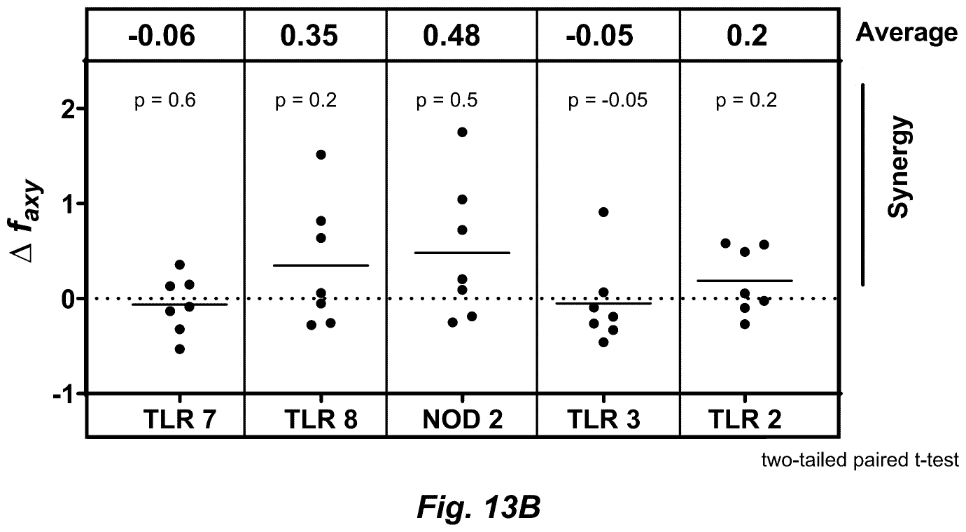

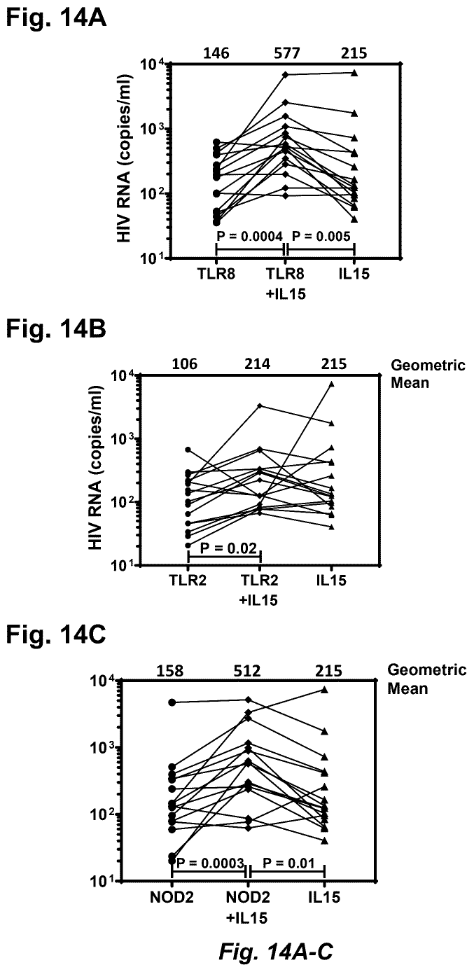

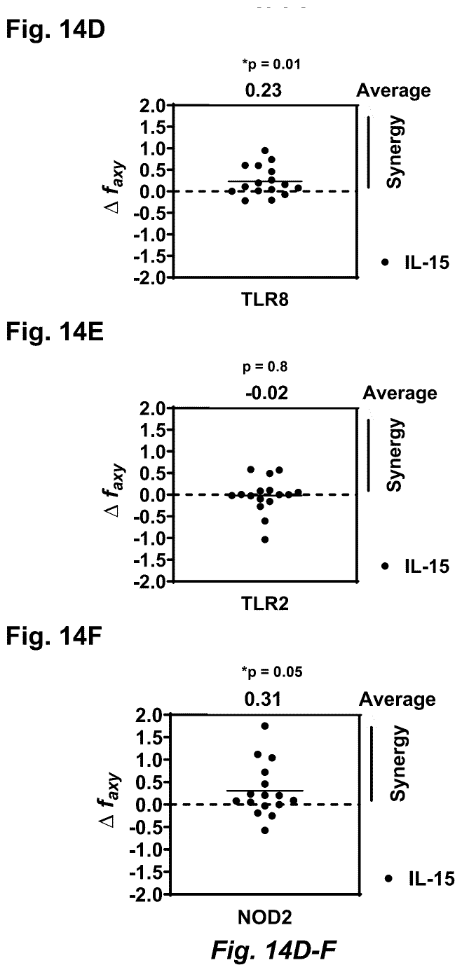

To determine if pattern recognition receptor agonists could further enhance HIV expression when combined with IL-15, each of the agonists that significantly induced HIV at least 2-fold were tested with or without IL-15 applying similar methods to those described above (N=7). TLR3 and TLR7 agonists did not further activate HIV when combined with IL-15, but TLR2, TLR8 and NOD2 agonists had additive activity when combined with IL-15 (FIG. 13A). In this limited dataset, combining a TLR2 agonist, a TLR8 agonist or a NOD2 agonist with IL-15 trended towards synergistic HIV activation calculated using Bliss independence model (Laird, et al., J Clin Invest. (2015) 125(5):1901-1912; and Bliss, Ann Appl Biol. (1939) 26:585-615), but such combinations were not significant (FIG. 13B). As there is substantial variability in the response to activating agents between donors, TLR2, TLR8 and NOD2 agonists were further tested performing the same methods in a larger set of donors (N=16) to see if the trends were consistent (FIG. 14). Statistically significant synergy, calculated using Bliss independence model, was observed when TLR8 and NOD2 were combined with IL-15 in this better powered set of donors (FIGS. 14D, 14E and 14F). This analysis showed that both TLR8 and NOD2 agonists were able to enhance HIV activation induced by IL-15, but that the effects seen with TLR2 and IL-15 were primarily driven by IL-15 alone.

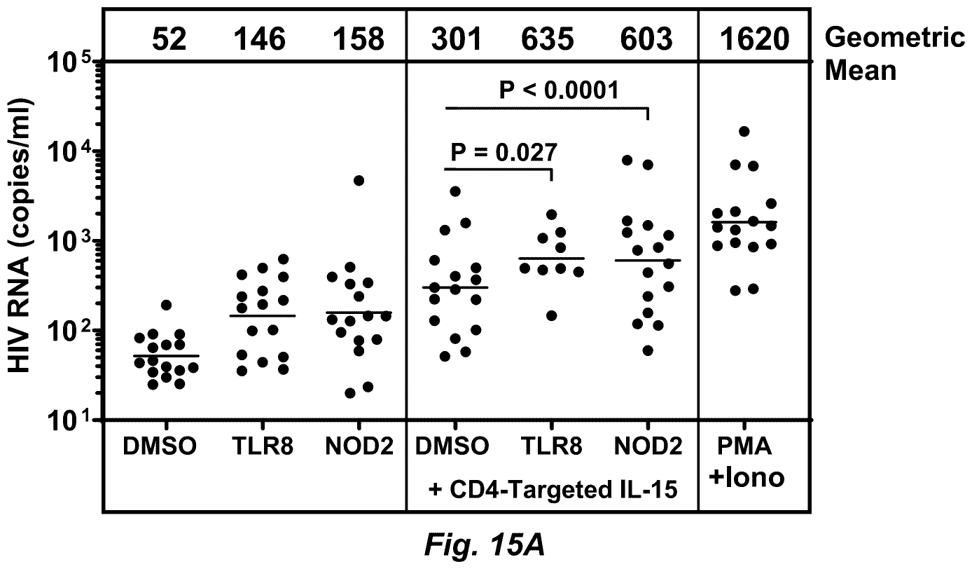

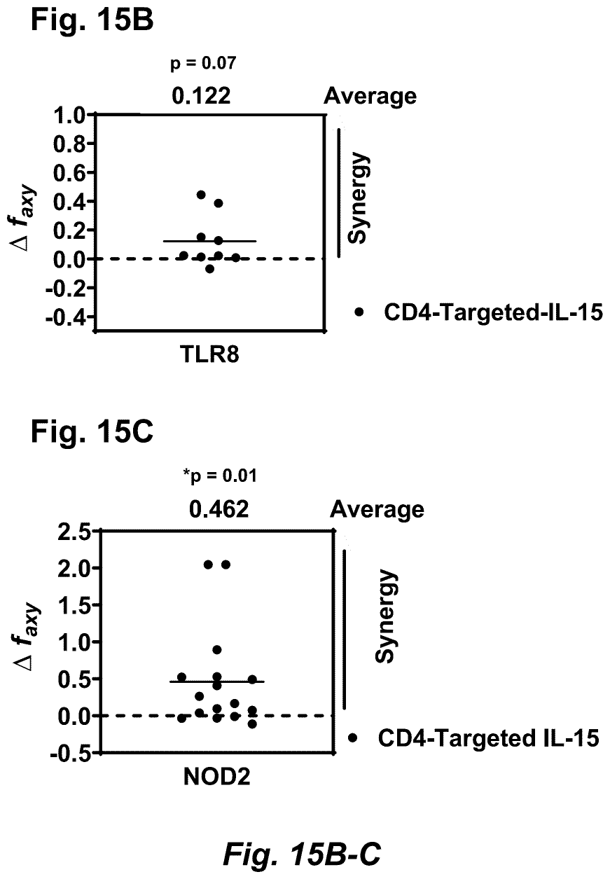

Having established that TLR8 and NOD2 agonists enhanced activation with IL-15, we tested if these agonists also enhanced HIV activation by CD4-targeted IL-15. For this analysis, the same methods were carried out as described above with the panel of pattern recognition receptor agonists. Selgantolimod (GS-9688) or romurtide were applied at 300 nM or 100 nM, respectively, and the CD4-targeted IL-15v (Rhesus surrogate Molecule 277, Table 20) was applied at 1 nM (N=16). FIG. 15 shows that activation with CD4-targeted IL-15v is stronger than previous results with recombinant human IL-15, and that TLR8 or NOD2 agonists further improve this HIV activation. The activation with each combination is significantly greater than that seen with each individual activator.

TABLE 20
CD4-Targeted IL-15v Molecule Used for TLR8 and NOD2 Agonist
Combination Studies

| Name | Molecule | Amino acid sequences |
|---|---|---|
| M1 anti- | 277 | SEQ ID NO: 1221 (heavy chain of Fab) |
| CD4 Fab | | EVKLVESGGGLVKPGGSLKLSCAASGFSFSNYAMSWVRQTPE |
| IL-15v | | KRLEWVASINDGGSTFYPDSVKGRFTISRDNAGNILYLQMSS |
| N4D/N65D | | LRSEDTAMFYCSRHYGGSYDPMDYWGQGTSVTVSSASTKGPS |
| Rhesus | | VFPLAPSSRSTSESTAALGCLVKDYFPEPVTVSWNSGSLTSG |
| surrogate | | VHTFPAVLQSSGLYSLSSVVTVPSSSLGTQTYVCNVNHKPSN |
| | | TKVDKRVEIKTCGGGSKPPTCPPCPAPEAAGGPSVFLFPPKP |
| | | KDTLMISRTPEVTCVVVDVKQEDPDVKFNWYVNGAEVHHAQT |
| | | KPRETQYNSTYRVVSVLTVTHQDWLNGKEYTCKVSNKALPAP |
| | | IQKTISKDKGQPREPQVYTLPPSREQLTKNQVKLTCLVKGFY |
| | | PSDIVVEWESSGQPENTYKTTPPVLDSDGSYFLYSKLTVDKS |
| | | RWQQGNVFSCSVMHEALHNHYTQKSLSVSPGK |
| | | SEQ ID NO: 1222 (light chain of Fab) |
| | | DIQMTQSPSSLSASLGGKVTITCKASQDINKYIAWYQHKPGK |
| | | GPRLLIHYTSTLHPGIPSRFSGSGSGRDYSFSISNLEPEDIA |
| | | TYYCLQYDNPLYTFGGGTKLEIKRAVAAPSVFIFPPSEDQVK |
| | | SGTVSVVCLLNNFYPREASVKWKVDGVLKTGNSQESVTEQDS |
| | | KDNTYSLSSTLTLSSTDYQSHNVYACEVTHQGLSSPVTKSFN |
| | | RGEC |
| | | SEQ ID NO: 1223 (Rα Sushi-IL-15v-Fc fusion) |
| | | ITCPPPVSVEHADIRVKSYSLYSRERYICNSGFKRKAGTSSL |
| | | TECVLNKATNIAHWTTPSLKCIRGGGGSGGGGSGGGGSGGGG |
| | | SGGGGSNWVDVISDLKKIEDLIQSMHIDATLYTESDVHPSCK |
| | | VTAMKCFLLELQVISHESGDTDIHDTVEDLIILANNILSSNG |
| | | NITESGCKECEELEEKNIKEFLQSFVHIVQMFINTSTSGGGS |
| | | KPPTCPPCPAPEAAGGPSVFLFPPKPKDTLMISRTPEVTCVV |
| | | VDVKQEDPDVKFNWYVNGAEVHHAQTKPRETQYNSTYRVVSV |
| | | LTVTHQDWLNGKEYTCKVSNKALPAPIQKTISKDKGQPREPQ |
| | | VYTLPPSREELTKNQVSLTCDVSGFYPSDIVVEWESSGQPEN |
| | | TYKTTPPVLDSDGSYFLYSKLTVDKSRWQQGNVFSCSVMHEA |
| | | LHNHYTQKSLSVSPGK |

### Example 8

### CD4-Targeted IL-15v Improves HIV Clearance by Reservoir Targeting Molecules

In this example, we evaluated if activation of HIV expression by CD4-targeted IL-15v molecules could enhance clearance of HIV in PBMCs isolated from ART-suppressed people with HIV (PWH). Several modalities have been developed that are designed to reduce HIV reservoirs by killing cells that express HIV proteins. However, these therapies may be ineffective against the majority of the reservoir due to low target expression. Previous studies show that the most HIV-infected cells in ART-suppressed PWH do not express HIV RNA (Wiegand et al., Proc Natl Acad Sci USA (2017) 114 (18): E3659-E3668). Activating HIV expression in these cells could increase the effectiveness of reservoir targeting molecules. In this example, we tested CD4-targeted IL-15v molecules with two different types of reservoir targeting molecules: (1) broadly neutralizing antibodies (bNAbs) that bind to HIV Env and recruit immune effector cells to HIV-infected cells (Bruel et al., Nat Comm (2016) 7:10844) and (2) HIV protease activators that promote dimerization of HIV pol in HIV-infected cells, prematurely activating HIV protease and inducing cell death (Wang et al., Science (2021) 371(6535):eabe1707).

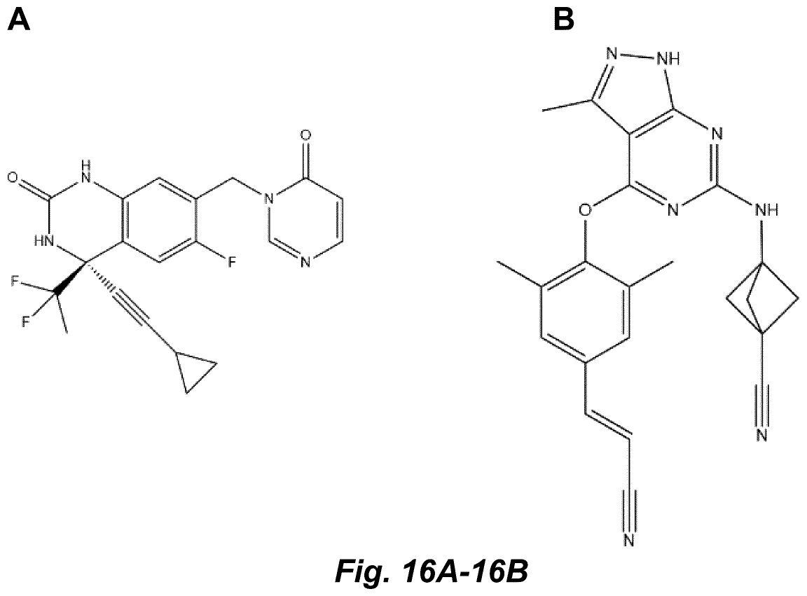

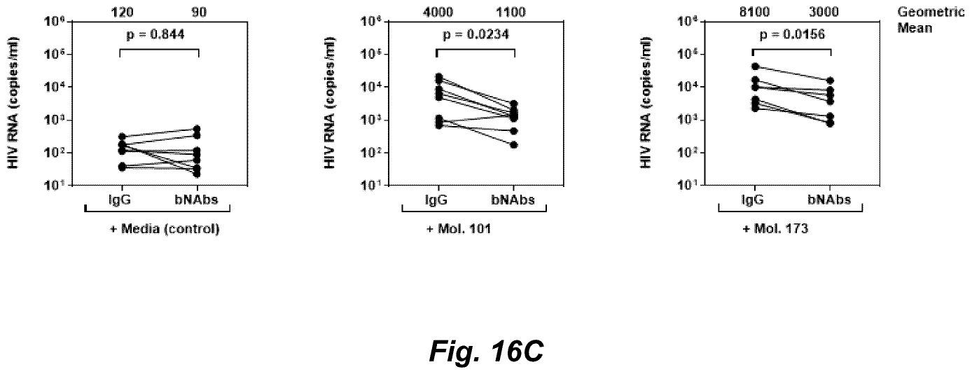

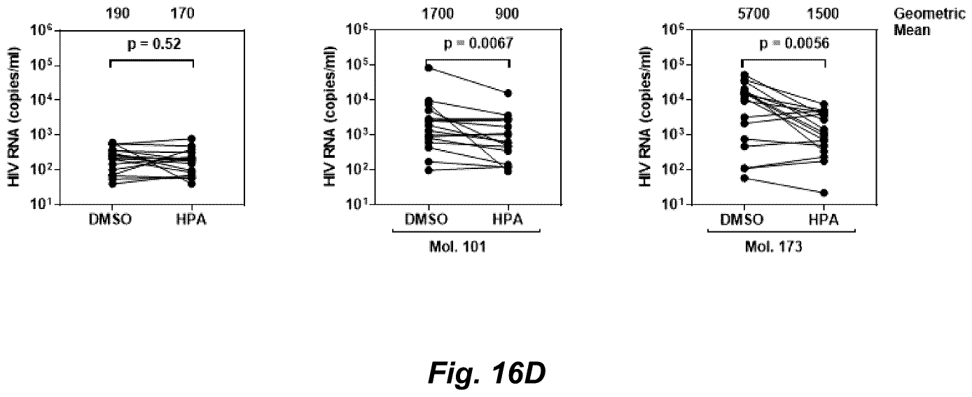

Peripheral blood mononuclear cells (PBMCs) were resuspended in RPMI 1640 media with 10% fetal bovine serum, penicillin-streptomycin and antivirals (100 nM elvitegravir and 100 nM efavirenz) at 3 million cells/ml. Cells were seeded at 300,000 per well in 96-well plates. Eight replicates per condition in ten replicate plates were treated with media control (no activation control), 1 nM CD4-targeted IL-15v (Molecule 101) in combination with human IgG control at 150 μg/ml or the HIV-specific bNAbs 3BNC117, PGT-121 and PGT-151 at 50 μg/ml each. CD4-targeted IL-15v Molecule 173 was applied in combination with human IgG control at 100 μg/ml or HIV specific bNAbs 10-1074 and 3BNC117 at 50 g/ml each. Cultures were similarly treated with DMSO control (vehicle control), CD4-targeted IL-15, 2 μM HIV protease activator, or the combination of CD4-targeted IL-15v and HIV protease activator (HPA). For Molecule 101, the HPA or non-nucleoside reverse transcriptase inhibitor (NNRTI) as targeted activator of cell kill (TACK), used in combination is depicted in FIG. 16A, and has been described in WO 2022/046844 (CAS Registry No. 2763257-44-7; IUPAC name: 2(1H)-Quinazolinone, 4-(2-cyclopropylethynyl)-4-(1,1-difluoroethyl)-6-fluoro-3,4-dihydro-7-[(6-oxo-1 (6H)-pyrimidinyl)methyl]-, (4S)-(ACI)). For the HPA (TACK) and Molecule 173 combination, the HPA molecule used in combination is depicted in FIG. 16B, has an IUPAC name of 3-[[4-[4-[(E)-2-cyanovinyl]-2,6-dimethyl-phenoxy]-3-methyl-1H-pyrazolo[3,4-d]pyrimidin-6-yl]amino]bicyclo[1.1.1]pentane-1-carbonitrile, and is described in co-pending, co-owned U.S. Appl. Nos. 63/560,148 and 63/560,405, which are hereby incorporated herein by reference in their entireties for all purposes. Treated cells were incubated for 7 days at 37° C. Cells were centrifuged and supernatants were collected. Supernatants from each of the ten replicate plates were pooled and HIV RNA levels were quantified using the COBAS®AmpliPrep/COBAS® TaqMan® HIV-1 Test, v2.0 (Roche) according to manufacturer's instructions. The geometric mean of eight replicates was calculated per condition for each donor (N=8 donors).

Results shown in FIG. 16 indicate that neither HIV bNAbs (FIG. 16C) nor HIV protease activators (FIG. 16D) reduce virion production in the absence of activation (media or vehicle control). Virion production was significantly reduced when CD4-targeted IL-15v was combined with bNAbs relative to the isotype control. Similarly, virion production was significantly reduced when CD4-targeted IL-15v was combined with the HIV protease activator. These results are consistent with increased reservoir clearance when CD4-targeted IL-15v is combined with reservoir targeting agents.

### Example 9

Compatibility of CD4-Targeted IL-15v Molecules with a CD4 Domain 1-Containing Bispecific T Cell Engager

In this example, we evaluated CD4-targeted IL-15v molecules that bind CD4 domain 1 or 3 for compatibility with a bispecific T cell engager that has a CD4 domain 1 variant to engage HIV, amtabafusp alfa (GS-8588) (described in WO 2022/046644A1).

The HIV-infected cell killing activity of the HIV T cell engager was evaluated using the CEM-NKr-CCR5-LucR+ lymphoblastoid reporter cell line (Spenlehauer et al., Virology (2001) 280(2):292-300). CEM-NKr-CCR5-LucR+ cells were seeded at 2 million cells/ml and incubated with 50 μg/ml DEAE dextran for 15 minutes at room temperature. Cells were infected with HIV BaL virus at a titer that gave >10-fold signal over background and incubated at 37° C. for 72 h. The HIV-Env binding bispecific T cell engager (amtabafusp alfa (GS-8588)) or control T cell engager that did not contain CD4 domain 1 (D1) and therefore would not bind HIV Env were added using serial dilutions with or without the CD4-targeted IL-15v molecule 101, 173, 175 or 182 (each at 30 times the CD4 proliferation EC50). Cells were added to each well at 20,000 cells per well and incubated for 1 hour. Human PBMCs were added to a final effector to target ratio of 5:1 and incubated for 48 hours. Expression of Tat-driven luciferase was quantified using Bright-Glo luciferase reagent (Promega). The percent infected cell killing was calculated using the following equation: 100-((RLU of HIV-infected targeted cells in treated wells/RLU HIV-infected target cells in untreated wells)*100). In similar experiments, the above protocol was used with a fixed concentration of amtabafusp alfa (GS-8588) at 1 μg/ml with and a dilution series of each CD4-targeted IL-15v molecule.

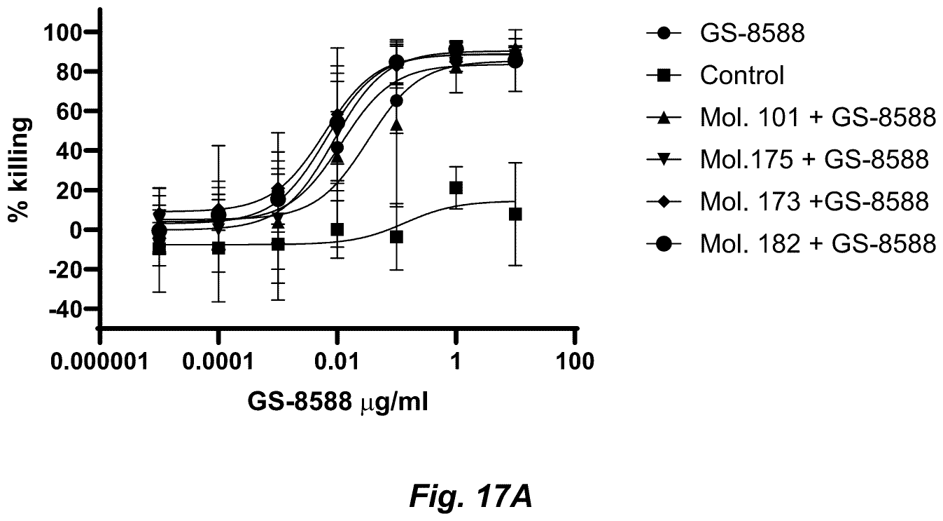

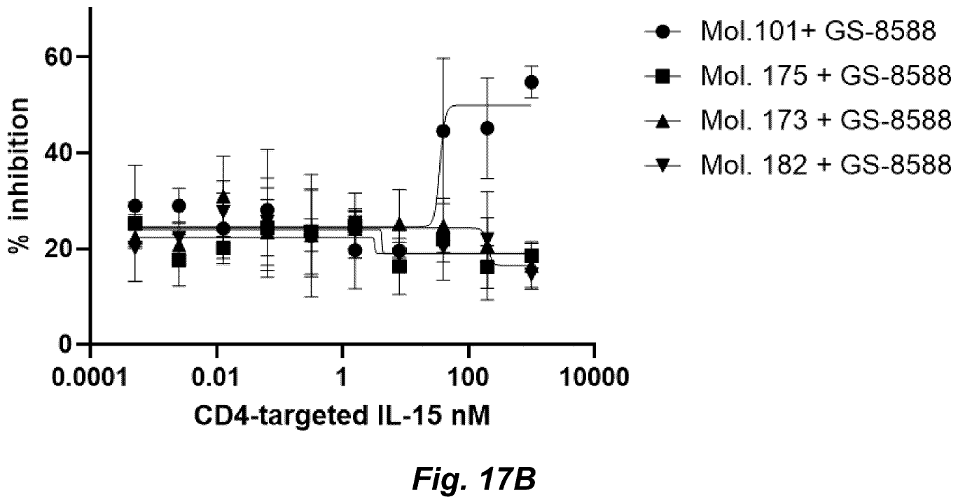

The results are shown in FIG. 17. The bispecific T cell engager amtabafusp alfa (GS-8588) induced dose dependent killing of HIV-infected cells with an EC50 of 0.012 μg/ml. The control molecule containing an anti-CD3 arm without CD4 domain 1 showed low background killing at the concentrations tested. At 30-times above the CD4 proliferation EC50, CD4-targeted IL-15v molecules did not significantly inhibit killing by the T cell engager, though the combination with Molecule 101 produced the lowest EC50 consistent with low level inhibition (see, FIG. 17A). In FIG. 17B, a fixed concentration of 1 μg/ml amtabafusp alfa (GS-8588) was used with a dose titration of CD4-targeted IL-15. Domain 3 (D3) binding CD4-targeted IL-15v (Molecules 173, 175 and 182) did not inhibit killing by amtabafusp alfa (GS-8588) at any of the doses tested. Domain 1 binding CD4-targeted IL-15v (Molecule 101) inhibited killing by amtabafusp alfa (GS-8588) at high doses, with an EC50 of 32.5 nM. These results confirm that at high doses, domain 1 binding CD4-targeted IL-15v molecules can inhibit reservoir targeting molecules that use CD4 domain 1 to engage HIV-infected cells. By contrast, the domain 3 binding CD4-targeted IL-15v molecules were fully compatible with a CD4 domain 1 containing reservoir targeting molecule.

### Example 10

### Expression of CD4-Targeted IL-15v Molecules

To evaluate the heterodimer product yield and homodimer impurity profiles in clonal cell lines, we transfected vectors encoding the three polypeptides (Fab heavy chain, Fab light chain and Ra Sushi Domain-IL-15v-Fc fusion, as presented in Table G) of a CD4-targeted IL15v into CHO-K1 GS (glutamine synthethase) knock-out host cells, plated 5000 transfected cells into each well of 96 well plates, and generated approximately 6000 stable mini-pools. After screening for titer and heterodimer/homodimer ratios, the top 10 mini-pools were advanced into the single-cell cloning stage. Approximately 200 single-cell-originated clones were generated from each mini-pool (for a total of ˜1000 clones), and these clones were then subjected to productivity and homodimer impurity screening, including the 14-day fed-batch assay, to identify the top-performing single-cell-derived clones. The fed-batch cultures were harvested on day 14, and the harvested cell culture fluid (HCCF) from each single-cell derived clone were used to determine overall titer, and then subjected to protein A affinity chromatography to purify and recover all protein A binding species (including CD4-IL15 heterodimer, CD4 homodimer, and IL15 homodimer). The protein A purified materials were then subjected to reversed-phase high performance liquid chromatography (RP-HPLC) to quantify the relative expression levels of the CD4-IL15 heterodimer product and the homodimer impurities.

As shown in Table 21, the top 10 single-cell-derived clones displayed very favorable growth characteristics (11 to 26 million cells/ml and >90% viability on day 14), very high productivity (6.1 to 9.5 g/L), and very low levels of homodimer impurities (less than 6% CD4 homodimer and less than 5% IL-15 homodimer). These clones are suitable for clinical and commercial production of the CD4-IL15v molecule.

TABLE 21
Growth Characteristics and Relative Levels of Heterodimer
and Homodimers Produced by Top Single-Cell-Derived Clones

| | | FB D 14 | | | | % Rα- |
|---|---|---|---|---|---|---|
| | FB D 14 VCD¹ | Viability | FB D 14 | % CD4-IL-15v | % CD4 Fab | IL-15v-Fc |
| Clone # | (e6 cells/mL) | (%) | Titer (g/L) | heterodimer | homodimer | homodimer |
| 1 | 21.5 | 97.0 | 9.3 | 93.6 | 1.4 | 5.0 |
| 4 | 26.0 | 93.5 | 9.2 | 95.7 | 3.1 | 1.2 |
| 8 | 12.2 | 92.0 | 7.2 | 96.6 | 2.5 | 0.9 |
| 9 | 13.0 | 93.6 | 8.6 | 96.2 | 2.6 | 1.2 |
| 10 | 11.6 | 94.0 | 8.6 | 96.5 | 2.0 | 1.5 |
| 11 | 12.2 | 97.0 | 9.1 | 95.6 | 3.5 | 1.0 |
| 13 | 12.2 | 95.3 | 7.0 | 95.7 | 2.9 | 1.3 |
| 14 | 11.9 | 95.1 | 8.8 | 95.9 | 3.3 | 0.8 |
| 25 | 20.3 | 94.2 | 9.5 | 94.0 | 6.0 | 0.0 |
| 40 | 20.5 | 90.9 | 6.1 | 96.2 | 3.1 | 0.6 |

¹FB D 14 VCD = Fed-batch day 14 viable cell density; E6 = 1 × 10⁶

### Example 11

Design of Rhesus Surrogate CD4-Targeted IL-15v Molecules and Assessment of CD4-targeted IL-15v Molecules in Cynomolgus PBMCs

To facilitate the in vivo study of CD4-targeted IL-15v molecules in non-human primates (NHPs), we generated surrogate molecules with the same architecture as described in Example 1, but where the component IL-15Rα, Il-15v and Fc amino acid sequences were based on rhesus rather than human origin. The simianized CD4-targeted IL-15v molecules contained a modified “knob-in-hole” heterodimer with F234A/L235A/P331S substitutions in both chains to eliminate effector function. Rhesus IL-15Rα-IL-15v was fused to a “knob” containing Fc domain and a CD4-specific binding domain was fused to the “hole” Fc domain. As with the human sequence molecules, flexible linkers tethered the IL-15Rα to IL-15v and IL-15v to the N-terminus of the Fc domain. Surrogate simianized CD4-targeted IL-15v molecules were designed with the N65D with or without the additional N4D substitution in IL-15v to reduce affinity for the IL-15 receptor (i.e. IL-2Rβγ).

The CD4-targeting domain used for these molecules was originally derived from a mouse antibody VH/VL sequences (M1 in Molecule 277; Table 20). To reduce the risk of anti-drug antibodies targeting murine sequences, this domain was simianized. The VH and VL sequences of the mouse monoclonal anti-CD4 antibody (M1) were compared to rhesus germlines sequences from IMGT (the international ImMunoGeneTic information systems). The rhesus germline sequence with the closet homology to the parental antibody was chosen as the acceptor sequence. The VH rhesus germline IGHV3-9 allele 1, VL rhesus germline IGKV1S8 allele 1 and IGKJ4 joining region sequence were used as the acceptor sequences. The CDRs were defined according to the AbM definition and improved for binding to human CD4. This modified CD4-binding domain was incorporated into the rhesus surrogate molecule (Molecule 247; Table H).

The CD4-targeted domain used for these molecules bound human CD4 more potently than rhesus CD4. For this reason, phage display was used to determine that a G54W substitution in the heavy chain and T531 and Y96Q substitutions in the light chain increased the affinity of the CD4-binding domain for rhesus CD4 so that it bound to human and rhesus CD4 with similar affinity. This binding domain was incorporated into a human CD4-targeted IL-15v molecule (Molecule 109; Table G).

These molecules were tested in cynomolgus PBMCs to determine potency and selectivity. Methods used were similar to those described in the PBMC proliferation assay in Example 1, but with the use of PBMCs isolated from cynomolgus macaques.

Table 22 shows that CD4-targeted IL-15v molecules have a range of potencies in cynomolgus CD4+ T cells and NK cells ranging from 0.0031 to 1.7 nM in CD4+ T cells and 4.6 to 240 nM in NK cells. Similarly, Table 23 shows a range of potencies in rhesus CD4+ T cells and NK cells ranging from 0.0015 to 0.74 nM in CD4+ T cells and 3.8 to 29 nM in NK cells. Molecules 101, 100 and 247 had decreased potency in NHP CD4+ T cells relative to human CD4+ T cells (see, results in Tables 7 and 12). By contrast, Molecules 109, 173 and 175 had similar or greater potency in NHP CD4+ T cells than in human CD4+ T cells (Tables 6 and 12). As seen in human cells, molecules with IgG4 Fc (101, 109, 175) were more potent than those with IgG1 Fc (100, 173, 247). Molecule 109 had the greatest potency in NHP CD4+ T cells with an EC50 of 0.0031 nM in cynomolgus and 0.0076 nM in rhesus PBMCs. The selectivity of these molecules in cynomolgus and rhesus cells supports the use of these molecules in NHP models.

TABLE 22
EC50 Values for Proliferation of CD4+ T Cells, CD8+ T Cells
and NK Cells Induced by IL-15-Fc Variants in CynomoIgus PBMCs

| | | | | | CD4 | CD8 | NK |
|---|---|---|---|---|---|---|---|
| | | CD4 | IL-15 | | EC50 | EC50 | EC50 |
| Molecule | Fab Name | domain | mutein | Fc | (nM) | (nM) | (nM) |
| 109 | Fab 10 (humanized M1) | D1 | N65D | IgG4 | 0.0031 | 130 | 6.0 |
| | G54W LC T53/Y96Q | | | | | | |
| 101 | Fab 7 (humanized M1) | D1 | N65D | IgG4 | 0.081 | 150 | 4.6 |
| 100 | Fab 7 (humanized M1) | D1 | N65D | IgG1 | 0.56 | 232 | 11 |
| 247 | M1 (simianized) | D1 | N65D | IgG1 | 1.7 | 160 | 6.1 |
| 173 | 1.22 | D3 | S7G/N65D | IgG1 | 0.34 | >1000 | 170 |
| 175 | 1.22 | D3 | N65D/I68A | IgG4 | 0.10 | >1000 | 240 |

TABLE 23
EC50 Values for Proliferation of CD4+ T Cells, CD8+ T Cells
and NK Cells Induced by IL-15-Fc Variants in Rhesus PBMCs

| | | | | | CD4 | CD8 | NK |
|---|---|---|---|---|---|---|---|
| | | CD4 | IL-15 | | EC50 | EC50 | EC50 |
| Molecule | Fab Name | domain | mutein | Fc | (nM) | (nM) | (nM) |
| 109 | Fab 10 (humanized M1) | D1 | N65D | IgG4 | 0.0076 | 52 | 3.8 |
| | G54W LC T53/Y96Q | | | | | | |
| | (humanized) | | | | | | |
| 101 | M1Fab 7 (humanized M1) | D1 | N65D | IgG4 | 0.064 | 77 | 6.79 |
| 100 | M1Fab 7 (humanized M1) | D1 | N65D | IgG1 | 0.37 | 216 | 18 |
| 247 | M1 (simianized) | D1 | N65D | IgG1 | 0.74 | >1000 | 29 |

### Example 12

Pharmacokinetics of CD4-Targeted IL-15v Molecules after Administration to Cynomolgus or Rhesus Macaques

In this example, we compared the single dose pharmacokinetics (PK) of multiple CD4-targeted IL-15v molecules in cynomolgus macaques.

Two untargeted IL-15-Fc fusion molecules (Molecules 263 and 264; amino acid sequences provided in Table 24) and six CD4-targeted IL-15v molecules (Molecules 100, 101, 109, 173, 175, and 247) were administered to protein naïve cynomolgus and rhesus macaques (N=3/group) (Labcorp, WI) via an intravenous (IV) bolus or following subcutaneous (SC) administration to characterize their PK profiles. Serial plasma samples collected from monkeys were analyzed using a selective bioanalytical method of sufficient sensitivity to determine plasma concentration-time profiles and mean plasma PK parameters by non-compartmental analysis (NCA). The bioanalytical method utilized CD4 protein as a capture reagent and detected following incubation with goat anti-human IgG1 biotin conjugated secondary antibody and ruthenylated conjugate streptavidin on a Mesoscale Discovery Quickplex SQ 120 plate reader. The calibration curve used the respective individual CD4-targeted IL-15v proteins as reference standards in spiked macaque matrix fit to a 4-parameter logistic model with 1/Y2 weighting. Analyte concentrations were determined from the ECL signals backfitted to the calibration curve. Plasma concentration-time profiles were used to calculate the mean plasma PK parameters by NCA.

TABLE 24
Untargeted-IL-15v Molecules Used in PK Studies

| Molecule | Amino acid sequences |
|---|---|
| 263 | SEQ ID NO: 1224 (RhIL15 RhFc1-C220S/E233P/L234V/L235A/ |
| | S267K/L368D/K370S) |
| | NWVNVISDLKKIEDLIQSMHIDATLYTESDVHPSCKVTAMKCFLLELQVISHES |
| | GDTDIHDTVENLIILANNILSSNGNITESGCKECEELEEKNIKEFLQSFVHIVQ |
| | MFINTSGGGGSEIKTSGGGSKPPTCPPCPAPPVAGPSVFLFPPKPKDTLMISRT |
| | PEVTCVVVDVKQEDPDVKFNWYVNGAEVHHAQTKPRETQYNSTYRVVSVLTVTH |
| | QDWLNGKEYTCKVSNKALPAPIQKTISKDKGQPREPQVYTLPPSREELTKNQVS |
| | LTCDVSGFYPSDIVVEWESSGQPENTYKTTPPVLDSDGSYFLYSKLTVDKSRWQ |
| | QGNVFSCSVMHEALHNHYTQKSLSVSPGK |
| | SEQ ID NO: 1225 (RhIL15Rα (Sushi) RhFc2-C220S/E233P/ |
| | L234V/L235A/S267K/S364K/E357Q) |
| | ITCPPPVSVEHADIRVKSYSLYSRERYICNSGFKRKAGTSSLTECVLNKATNIA |
| | HWTTPSLKCIRGGGGSEIKTSGGGSKPPTCPPCPAPPVAGPSVFLFPPKPKDTL |
| | MISRTPEVTCVVVDVKQEDPDVKFNWYVNGAEVHHAQTKPRETQYNSTYRVVSV |
| | LTVTHQDWLNGKEYTCKVSNKALPAPIQKTISKDKGQPREPQVYTLPPSREQLT |
| | KNQVKLTCLVKGFYPSDIVVEWESSGQPENTYKTTPPVLDSDGSYFLYSKLTVD |
| | KSRWQQGNVFSCSVMHEALHNHYTQKSLSVSPGK |
| 264 | SEQ ID NO: 1226 (Rh IL15 D30N/E64Q/N65D, RhFc1- |
| | C220S/E233P/L234V/L235A/S267K/L368D/K370S/M428L/N434S) |
| | NWVNVISDLKKIEDLIQSMHIDATLYTESNVHPSCKVTAMKCFLLELQVISHES |
| | GDTDIHDTVQDLIILANNILSSNGNITESGCKECEELEEKNIKEFLQSFVHIVQ |
| | MFINTSGGGGSEIKTSGGGSKPPTCPPCPAPPVAGPSVFLFPPKPKDTLMISRT |
| | PEVTCVVVDVKQEDPDVKFNWYVNGAEVHHAQTKPRETQYNSTYRVVSVLTVTH |
| | QDWLNGKEYTCKVSNKALPAPIQKTISKDKGQPREPQVYTLPPSREELTKNQVS |
| | LTCDVSGFYPSDIVVEWESSGQPENTYKTTPPVLDSDGSYFLYSKLTVDKSRWQ |
| | QGNVFSCSVLHEALHSHYTQKSLSVSPGK |
| | SEQ ID NO: 1227 (Rh IL15Ra (Sushi) RhFc2-C220S/E233P/ |
| | L234V/L235A/S267K/S364K/E357Q/M428L/N434S |
| | ITCPPPVSVEHADIRVKSYSLYSRERYICNSGFKRKAGTSSLTECVLNKATNIA |
| | HWTTPSLKCIRGGGGSEIKTSGGGSKPPTCPPCPAPPVAGPSVFLFPPKPKDTL |
| | MISRTPEVTCVVVDVKQEDPDVKFNWYVNGAEVHHAQTKPRETQYNSTYRVVSV |
| | LTVTHQDWLNGKEYTCKVSNKALPAPIQKTISKDKGQPREPQVYTLPPSREQLT |
| | KNQVKLTCLVKGFYPSDIVVEWESSGQPENTYKTTPPVLDSDGSYFLYSKLTVD |
| | KSRWQQGNVFSCSVLHEALHSHYTQKSLSVSPGK |

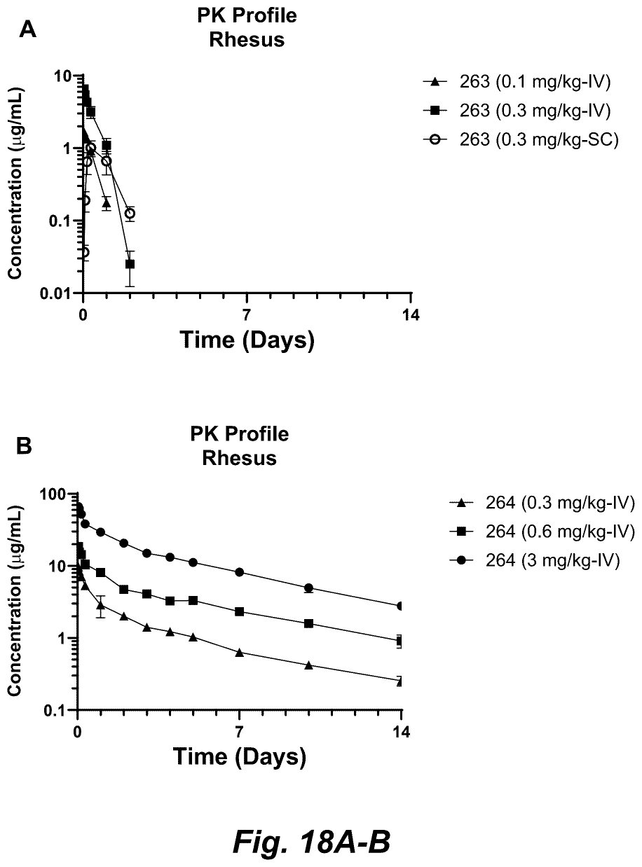

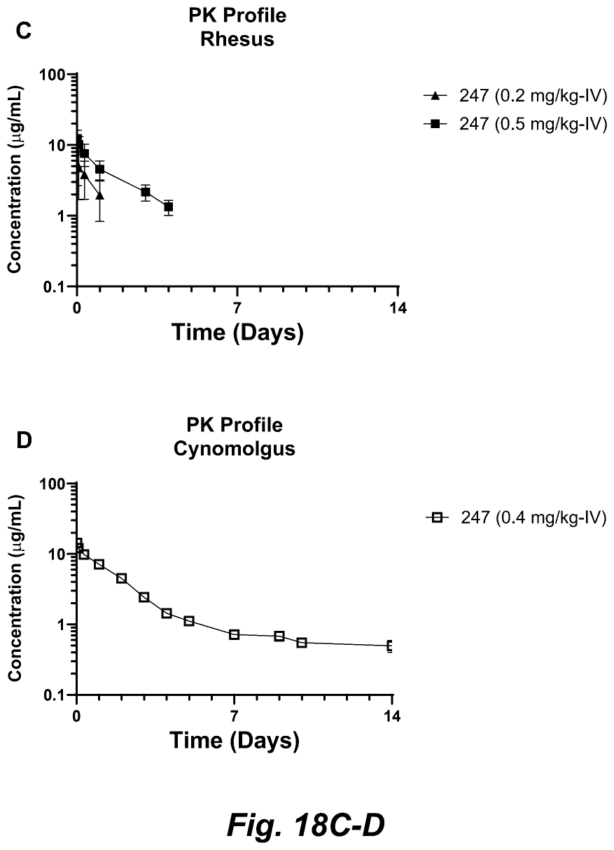

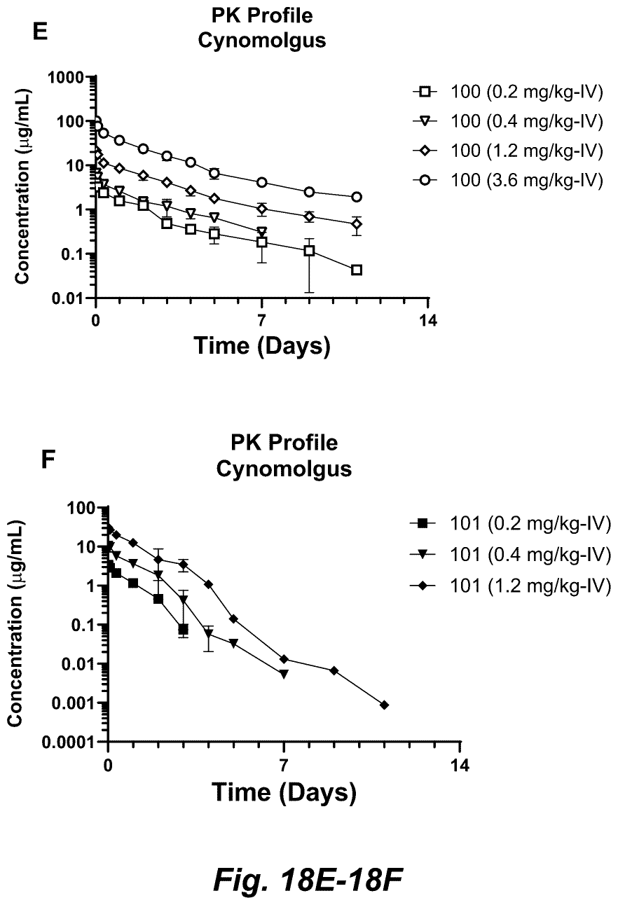

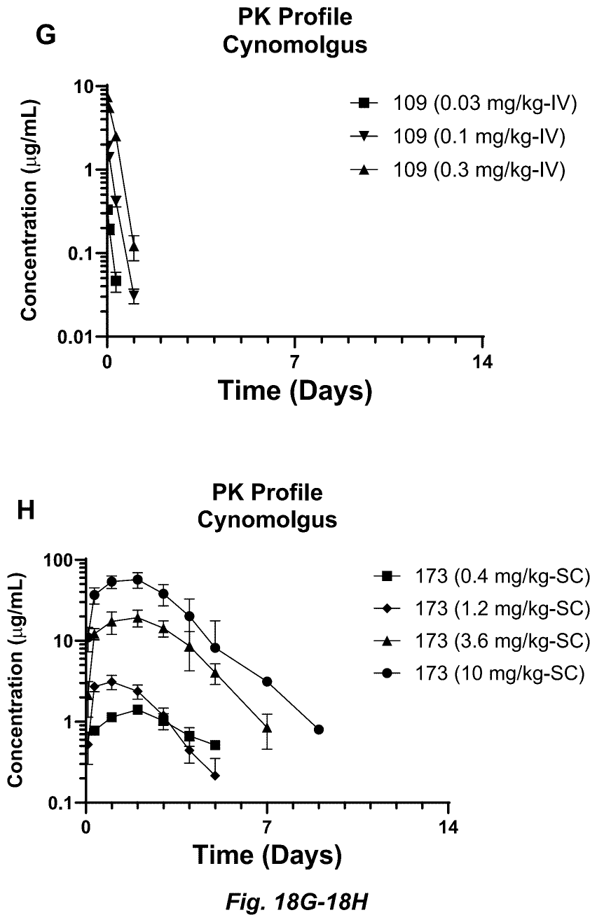

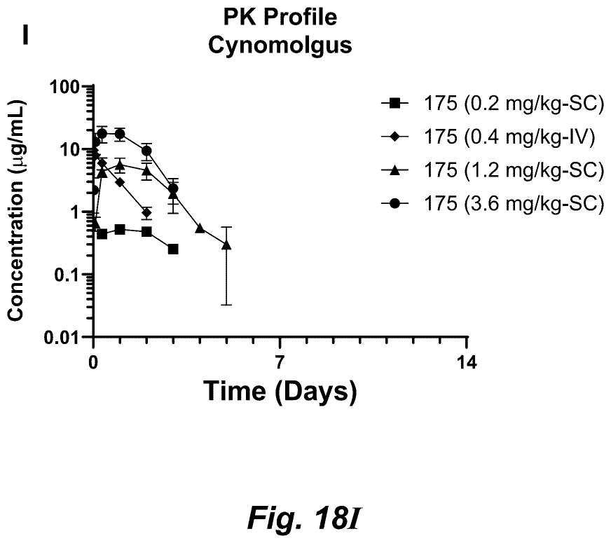

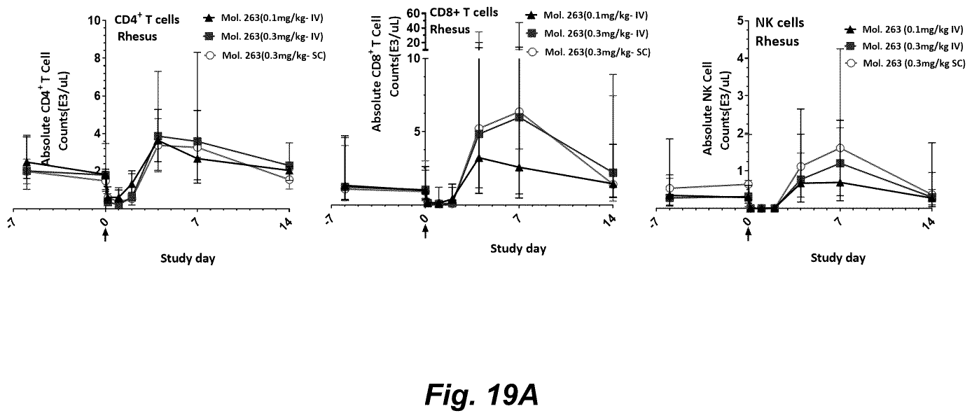

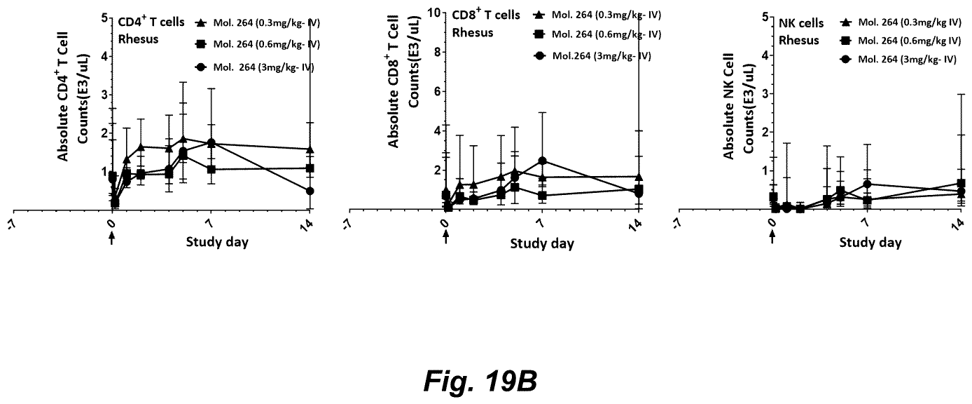

The results of this analysis demonstrated that the CD4-targeted IL-15v molecules (100, 101, 109, 173, 175, 247, 263 and 264) exhibited target mediated disposition profile. The serum concentration-time profiles are presented in FIGS. 18A-18I and summarized in Table 25. As previously reported, target mediated clearance is directly related to the potency of untargeted IL-15 molecules (Lu et al., Eur J Pharm Sci (2023) 186:106450). The decreased potency of 264 is associated with longer exposure times. For CD4-targeted IL-15v molecules, the more potent 109 was the most rapidly cleared (FIG. 18G). lgG4-based Fc molecules (FIGS. 18F and 18I for Molecules 101 and 175, respectively) were also cleared more rapidly than IgG1-based Fc molecules (FIGS. 18E and 18H for Molecules 100 and 173, respectively. This is consistent with the increased potency observed for IgG4-based Fc molecules relative to IgG1-based Fc molecules described in the PBMC Proliferation Assay in Example 1. These results are consistent with the conclusion that the desired potency for CD4-targeted IL-15v molecules is high enough to effectively activate CD4+ T cells, but also low enough to achieve the extended exposure likely required to achieve the desired activation with a single administration.

TABLE 25

| | Dose | Admin | Cmax | AUClast | CI | Half-Life |
|---|---|---|---|---|---|---|
| Molecule | (mg/kg) | Route | (μg/mL) | (day*μg/mL) | (mL/day/kg) | (day) |
| 263 | 0.1 | IV | 1.66 ± 0.0646 | 0.799 ± 0.0372 | 115 ± 5.61 | 0.288 ± 0.0345 |
| 263 | 0.3 | IV | 6.57 ± 0.665 | 3.55 ± 0.532 | 85.4 ± 12.1 | 0.253 ± 0.0285 |
| 263 | 0.3 | SC | 1.01 ± 2.48 | 1.13 ± 0.345 | — | — |
| 264 | 0.3 | IV | 9.61 ± 0.278 | 16.4 ± 1.01 | 16.5 ± 1.25 | 4.98 ± 0.556 |
| 264 | 0.6 | IV | 18.7 ± 1.83 | 45.4 ± 3.75 | 11.6 ± 1.31 | 5.12± 0.826 |
| 264 | 3 | IV | 66.6 ± 8.83 | 165 ± 7.31 | 16.5 ± 0.863 | 4.48 ± 0.302 |
| 247 | 0.2 | IV | 5.35 ± 2.70 | 4.25 ± 4.7 | 40.06 ± 22.35 | 0.902 ± 0.217 |
| 247 | 0.5 | IV | 12.3 ± 3.84 | 15 ± 4.82 | 29.21 ± 7.595 | 1.43 ± 0.148 |
| 247 | 0.4 | IV | 14.3 ± 0.104 | 27.8 ± 1.67 | 12.9 ± 0.220 | 4.70 ± 3.40 |
| 100 | 0.2 | IV | 4.17 ± 0.930 | 3.51 ± 0.561 | 57.3 ± 10.3 | 1.54 ± 0.843 |
| 100 | 0.4 | IV | 7.74 ± 1.25 | 10.7 ± 2.78 | 37.4 ± 12.6 | 1.47 ± 0.499 |
| 100 | 1.2 | IV | 23.8 ± 1.29 | 41.6 ± 3.91 | 27.8 ± 2.40 | 1.98 ± 0.194 |
| 100 | 3.6 | IV | 102 ± 9.82 | 146 ± 6.55 | 23.4 ± 0.894 | 2.75 ± 1.13 |
| 101 | 0.2 | IV | 0.0868 ± 0.0553 | 0.209 ± 0.068 | 251 ± 53.3 | 0.664 ± 0.168 |
| 101 | 0.4 | IV | 11.613 ± 2.286 | 14.714 ± 2.844 | 26.707 ± 5.261 | 1.232 ± 0.390 |
| 101 | 1.2 | IV | 31.3 ± 2.17 | 36.1 ± 6.63 | 33.4 ± 6.14 | 0.703 ± 0.242 |
| 109 | 0.03 | IV | 0.333 ± 0.0252 | 0.0541 ± 0.0058 | 490 ± 66.8 | — |
| 109 | 0.1 | IV | 1.87 ± 0.0646 | 0.523 ± 0.0646 | 191 ± 25.6 | 0.166 ± 0.005 |
| 109 | 0.3 | IV | 7.40 ± 0.923 | 2.19 ± 0.682 | 125 ± 17.7 | 0.177 ± 0.0266 |
| 173 | 0.4 | SC | 1.08 ± 0.269 | 2.97 ± 1.01 | — | — |
| 173 | 1.2 | SC | 3.12 ± 0.632 | 7.86 ± 1.66 | 149 ± 34.9 | 0.855 ± 0.097 |
| 173 | 3.6 | SC | 19.3 ± 4.50 | 69.7 ± 18.4 | 53.1 ± 12.3 | 0.897 ± 0.017 |
| 173 | 10 | SC | 58.7 ± 11.7 | 174 ± 33.2 | 63.4 ± 9.17 | 0.564 ± 0.0741 |
| 175 | 0.2 | SC | 0.523 ± 0.0295 | 1.01 ± 0.179 | — | — |
| 175 | 0.4 | IV | 9.62 ± 0.640 | 7.32 ± 0.984 | 49.3 ± 6.86 | 0.646 ± 0.0586 |
| 175 | 1.2 | SC | 5.65 ± 1.49 | 13.6 ± 3.86 | 88.1 ± 20.5 | 0.725 ± 0.05 |
| 175 | 3.6 | SC | 19.0 ± 4.88 | 49.1 ± 12.3 | 73.0 ± 20.8 | 0.818 ± 0.193 |

### Example 13

Immune Activation after Administration of Untargeted and CD4-Targeted IL-15v Molecules

In this example, we assessed the in vivo effect of untargeted IL-15 and CD4-targeted IL-15v molecules on activation of T cells and NK cells, which are naturally stimulated by IL-15. We also measured plasma cytokine and chemokine levels after treatment with IL-15 and CD4-targeted IL-15v.

As described in Example 12, two untargeted IL-15-Fc fusion molecules (Molecules 263 and 264) and six CD4-targeted IL-15v molecules (Molecules 100, 101, 109, 173, 175, and 247) were administered to protein naïve cynomolgus and rhesus macaques (N=3/group) (Labcorp, WI) via an intravenous (IV) bolus or following subcutaneous (SC) administration.

Immune cell activation was monitored by collecting whole blood in EDTA tubes at different time points during the study. Changes to immune cell subsets were evaluated by flow cytometry and HEMAVET based CBC (Complete Blood Count) analysis. Frequencies of T cells (CD3+CD4+ and CD3+CD8+) and NK cells (CD3-CD14-CD20-CD8+NKG2a+) were obtained from the parent CD45+ lymphocyte gate and combined with lymphocyte frequencies determined by complete blood count analysis to calculate absolute cell counts.

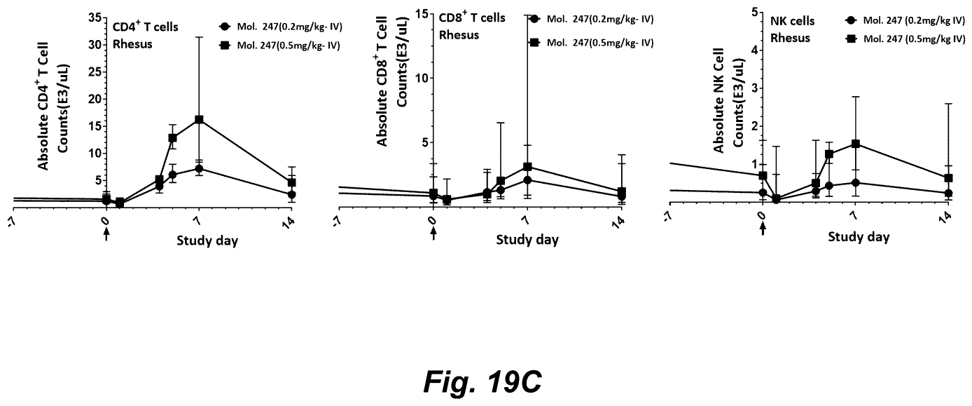

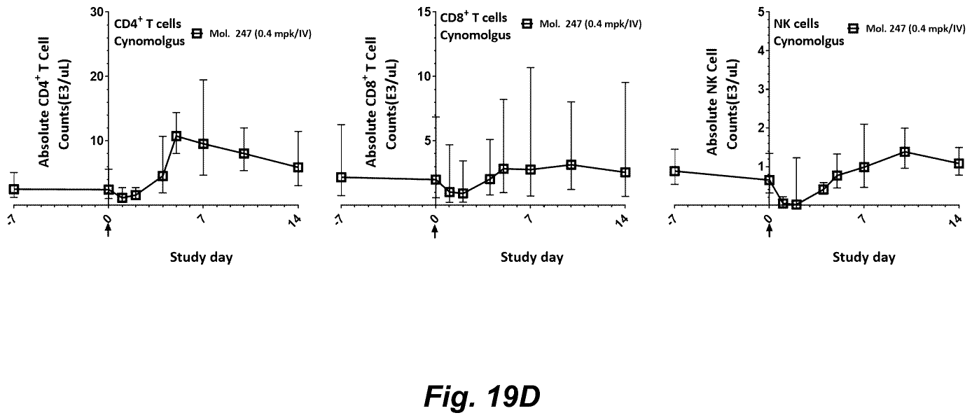

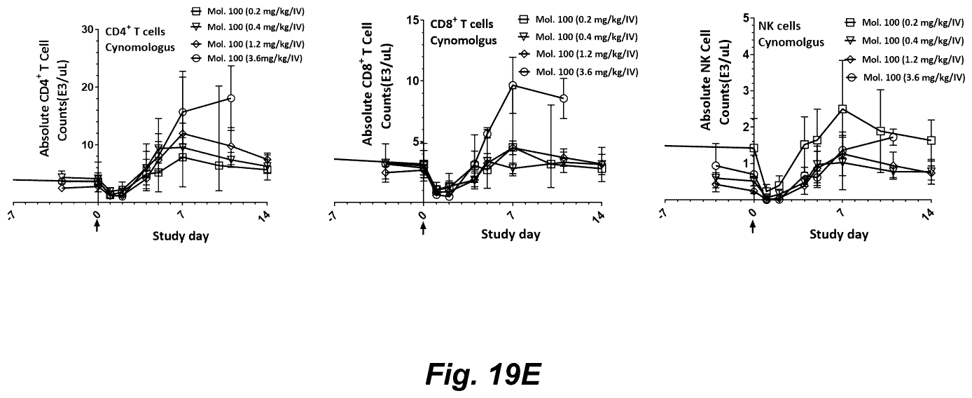

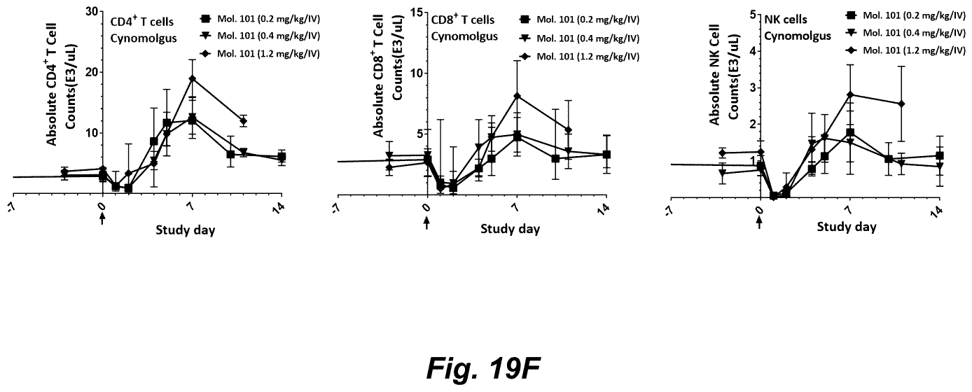

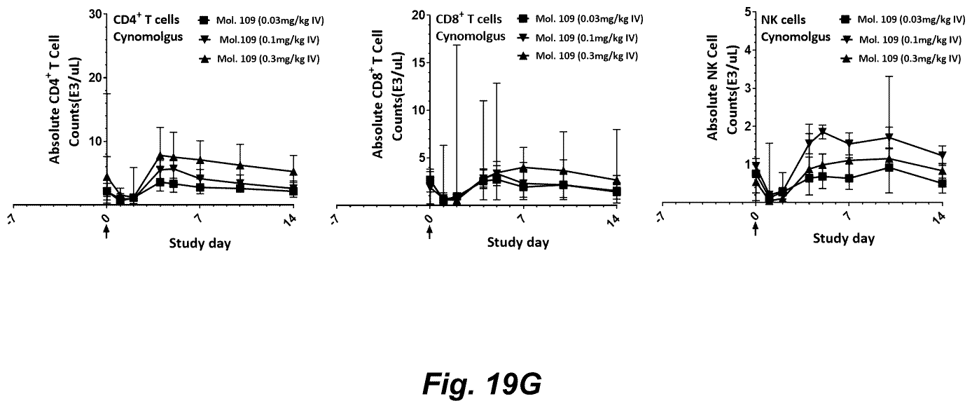

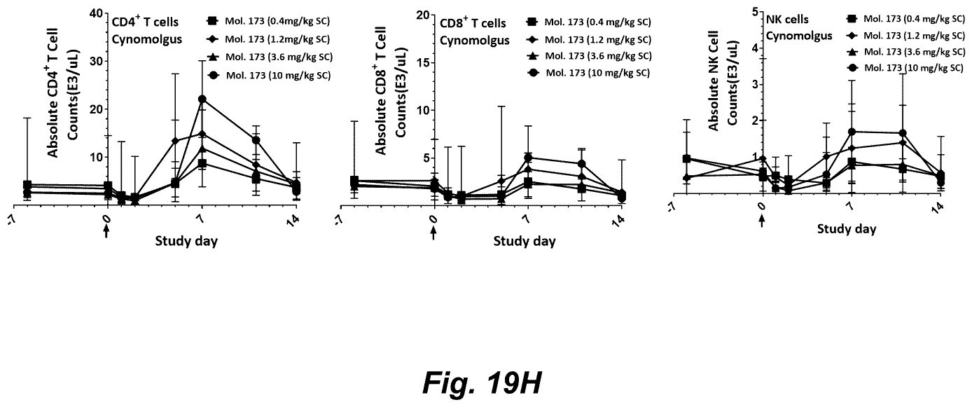

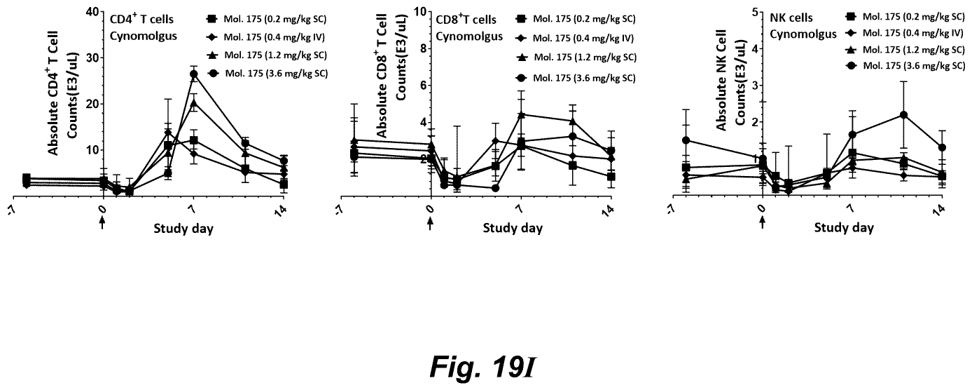

The results are shown in FIGS. 19A-19I. One day after dosing with each molecule, there was a rapid transient decline in peripheral lymphocyte numbers, which has also been seen after dosing with IL-15 or IL-2 in human trials (Conlon, et al., J Clin Oncol. (2015) 33(1):74-82; and Lotze, et al., J Immunol. (1985) 135(4):2865-75). This is consistent with lymphocyte trafficking to tissues. By four days, T cells and NK cells returned to baseline in the periphery or began to increase by day four. For the untargeted molecules, NK activation was stronger than T cell activation (FIGS. 19A and 19B).

Most CD4-targeted IL-15v molecules induced greater activation of CD4+ T cells than either of the untargeted molecules. The exception was Molecule 109 (FIG. 19G), which induced lower CD4 activation than the closely related Molecule 101 (FIG. 19F). Molecule 109 was more potent and had more rapid clearance than Molecule 101, which appeared to limit its effectiveness after a single dose. This shows that molecules with high potency not only have decreased exposure due to target mediated drug disposition, but also are less effective at activating CD4+ T cells after a single dose. Molecules 247, 100, 101, 173 and 175 induce strong activation of CD4+ T cells without increasing CD8+ T cells or NK cells relative to the levels induced by untargeted IL-15.

Previous studies have shown that IL-15 agonists can induce immunotoxicity through the activation of IFN-γ that is produced by NK cells (Guo et al., J Immunol (2015) 195:2353-2364). Specific targeting of IL-15 to CD4+ cells can mitigate this toxicity, enabling safe administration of IL-15 at doses that are high enough to activate latent HIV.

Plasma was collected at various time points after dosing with Molecules 100, 101, 173, 247 and 263 and analyzed by multiplex analysis for interferon-gamma (IFN-γ), C-X-C motif chemokine ligand 11 (I-TAC; CXCL11), interleukin 1 receptor antagonist (IL-1RA; IL-1RN), interleukin 6 (IL-6), C-C motif chemokine ligand 2 (MCP-1; CCL2), C-X-C motif chemokine ligand 9 (MIG; CXCL9) and C-C motif chemokine ligand 4 (MIP-1β; CCL4) (ProcartaPlex Luminex kits, Thermo Fisher Scientific) according to manufacturer's instructions. Table 26 shows results for pre-dose levels and 48 h post-dose for the indicated cytokines and chemokines.

TABLE 26
Cytokine Levels Induced by Untargeted and CD4-Targeted
IL-15v Molecules in Rhesus or CynomoIgus Macaques

| Molecule | Dose/Route | | Study time | IFN-γ | I-TAC | IL-1 RA | IL-6 | MCP-1 | MIG | MIP-1β |
|---|---|---|---|---|---|---|---|---|---|---|
| 263 | 0.1 | mg/kg/IV | Baseline | 2 | 15 | 81 | 30 | 108 | 6 | 11 |
| | | | 48 hr PD | 5 | 261 | 754 | 34 | 336 | 116 | 15 |
| 263 | 0.3 | mg/kg/IV | Baseline | 3 | 16 | 136 | 48 | 148 | 11 | 13 |
| | | | 48 hr PD | 26 | 261 | 3133 | 31 | 466 | 91 | 18 |
| 263 | 0.3 | mg/kg/SC | Baseline | 3 | 49 | 121 | 86 | 94 | 25 | 21 |
| | | | 48 hr PD | 37 | 481 | 2460 | 102 | 475 | 153 | 39 |
| 247 | 0.2 | mg/kg/IV | Baseline | 2 | 9 | 57 | 8 | 37 | 14 | 23 |
| | | | 48 hr PD | 2 | 10 | 60 | 8 | 78 | 14 | 14 |
| 247 | 0.5 | mg/kg/IV | Baseline | 2 | 12 | 57 | 8 | 102 | 10 | 24 |
| | | | 48 hr PD | 2 | 12 | 68 | 9 | 228 | 10 | 23 |
| 247 | 0.4 | mg/kg/IV | Baseline | 2 | 3 | 58 | 8 | 38 | 9 | 46 |
| | | | 48 hr PD | 2 | 11 | 58 | 8 | 162 | 9 | 77 |
| 100 | 0.2 | mg/kg/IV | Baseline | 2 | 11 | 54 | 8 | 16 | no data | 10 |
| | | | 48 hr PD | 2 | 11 | 54 | 8 | 16 | no data | 8 |
| 100 | 0.4 | mg/kg/IV | Baseline | 2 | 3 | 14 | 8 | 22 | 9 | 10 |
| | | | 48 hr PD | 2 | 5 | 36 | 8 | 75 | 9 | 21 |
| 100 | 1.2 | mg/kg/IV | Baseline | 2 | 8 | 55 | 8 | 8 | 13 | 14 |
| | | | 48 hr PD | 2 | 8 | 69 | 8 | 16 | 13 | 22 |
| 100 | 3.6 | mg/kg/IV | Baseline | 2 | 3 | 15 | 8 | 74 | 9 | 29 |
| | | | 48 hr PD | 5 | 9 | 260 | 19 | 103 | 14 | 42 |
| 101 | 0.2 | mg/kg/IV | Baseline | 2 | 11 | 71 | 8 | 24 | 8 | 10 |
| | | | 48 hr PD | 2 | 11 | 82 | 8 | 64 | 8 | 9 |
| 101 | 0.4 | mg/kg/IV | Baseline | 3 | 14 | 164 | 21 | 18 | 8 | 8 |
| | | | 48 hr PD | 3 | 16 | 211 | 16 | 32 | 8 | 8 |
| 101 | 1.2 | mg/kg/IV | Baseline | 2 | 13 | 54 | 8 | 26 | 8 | 15 |
| | | | 48 hr PD | 2 | 15 | 355 | 8 | 23 | 10 | 16 |
| 173 | 0.4 | mg/kg/SC | Baseline | 2 | 13 | 54 | 11 | 13 | 5 | 13 |
| | | | 48 hr PD | 2 | 13 | 60 | 11 | 12 | 5 | 13 |
| 173 | 1.2 | mg/kg/SC | Baseline | 2 | 13 | 54 | 11 | 15 | 5 | 13 |
| | | | 48 hr PD | 2 | 13 | 59 | 11 | 55 | 5 | 13 |
| 173 | 3.6 | mg/kg/SC | Baseline | 2 | 13 | 54 | 11 | 11 | 5 | 22 |
| | | | 48 hr PD | 2 | 13 | 54 | 11 | 55 | 9 | 28 |
| 173 | 10 | mg/kg/SC | Baseline | 2 | 13 | 66 | 11 | 82 | 5 | 103 |

| | | | | | | | |
|---|---|---|---|---|---|---|---|
| 48 hr PD | 4 | 13 | 591 | 11 | 251 | 5 | 54 |

Values represent geomean (pg/ml) from 3 animals dosed for each treatment condition.
Values that are ≥3-fold at 48-hour post dosing above the pre-dose values are shown in bold.

The results shown in Table 26 show that untargeted IL-15 (Molecule 263) induced five measured cytokines ≥3-fold above the pre-dose levels at 48 h post-dose, including IFN-γ, I-TAC and IL-1RA, MCP-1 and MIG. Of these, CD4-targeted IL-15v molecules only induced MCP-1 at doses that were specific for CD4+ T cells. At the highest doses tested, where there was also some detectable expansion of CD8+ T cells and NK cells, there was also induction of I-TAC and/or IL-1RA for some CD4-targeted IL-15v molecules. These results are consistent with an improved safety profile for CD4-targeted IL-15v molecules at doses that activate CD4+ T cells relative to untargeted IL-15 molecules.

### Example 14

### CD4-Targeted IL-15v Increases Viral Expression in ART-Suppressed, Chronically SIV-Infected Rhesus Macaques

In this example, we assessed the ability of CD4-targeted IL-15v to activate SIV expression in ART-suppressed rhesus macaques chronically infected with SIVmac251.

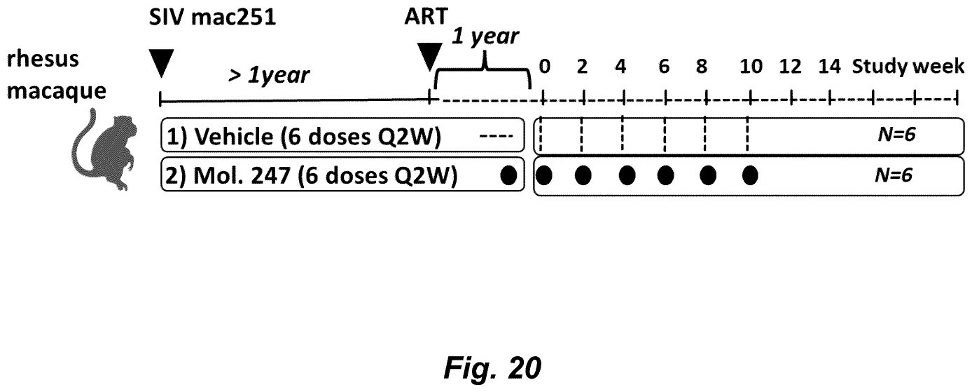

Twelve rhesus macaques were infected with SIVmac251 for 1 year and then treated with daily ART for 12 months prior to dosing with CD4-targeted IL-15v. Animals were distributed into two groups based on pre-ART viral load set points, CD4 counts and body weights. Six animals were dosed in each group: group 1 (g1) vehicle buffer and group 2 (g2) 0.5 mg/kg Molecule 247, intravenously (IV) for a total of 6 biweekly doses in the presence of ART. The study design is shown in FIG. 20.

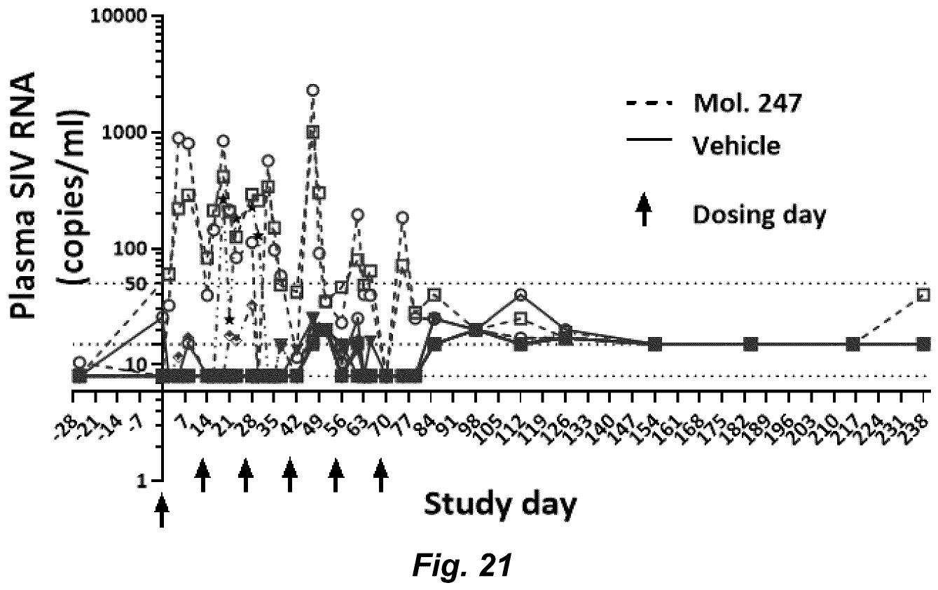

Plasma SIV viral RNA was assessed throughout the study duration utilizing SIV Gag RNA-specific quantitative reverse transcriptase polymerase chain reaction (qRT-PCR) during dosing and up to 6 months after the last dose (FIG. 21). PCR assays with 8 or 15 copy/ml lower limits of quantification (LLOQs) were utilized. Fifteen copy limit assays were used from days 47 to 51 and again from days 85 to 238. Viral loads are shown for individual animals in the vehicle only (solid lines) and Molecule 247 (dashed lines) groups. Plasma SIV RNA remained below 50 copies/ml in all animals in the vehicle control group. By contrast, Molecule 247 increased plasma SIV RNA above 50 copies/ml in 3 of 6 treated animals.

In this study, CD4+ T cell proliferation was measured by flow cytometry after the first, fourth and sixth doses of Molecule 247. Whole blood was collected in EDTA tubes and HEMAVET (hematology analyzer)-based complete blood counts (CBC) analysis was completed. Frequencies of CD4+ T cells (CD3+CD4+) were obtained from the parent CD45+lymphocyte gate and combined with lymphocyte frequencies determined by complete blood count analysis to calculate absolute CD4+ T cell counts. Central memory CD4+ T cells (CD3+CD4+CD95+CD28+) and naive CD4+ T cells (CD3+CD4+CD95-CD28+) were gated from parent CD4+ T cells. Proliferation was assessed by staining for Ki67 and gating on Ki67+ cells within these populations.

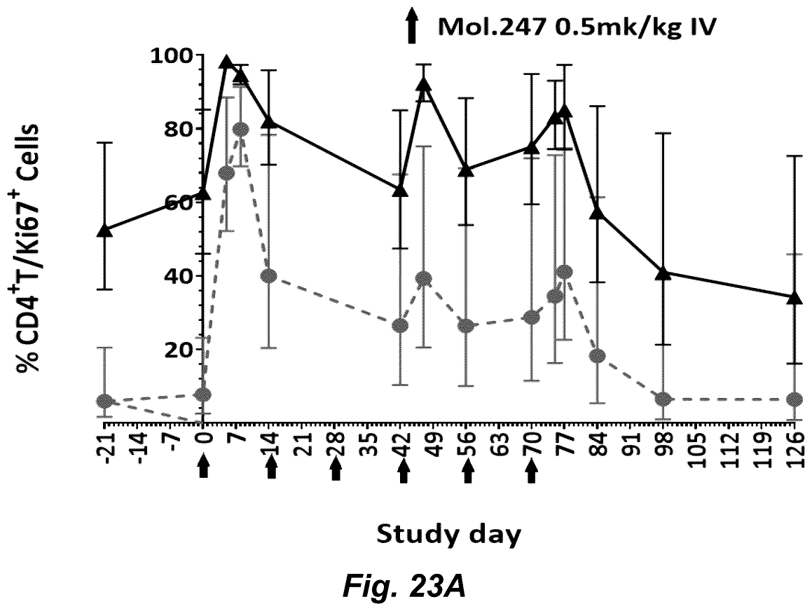

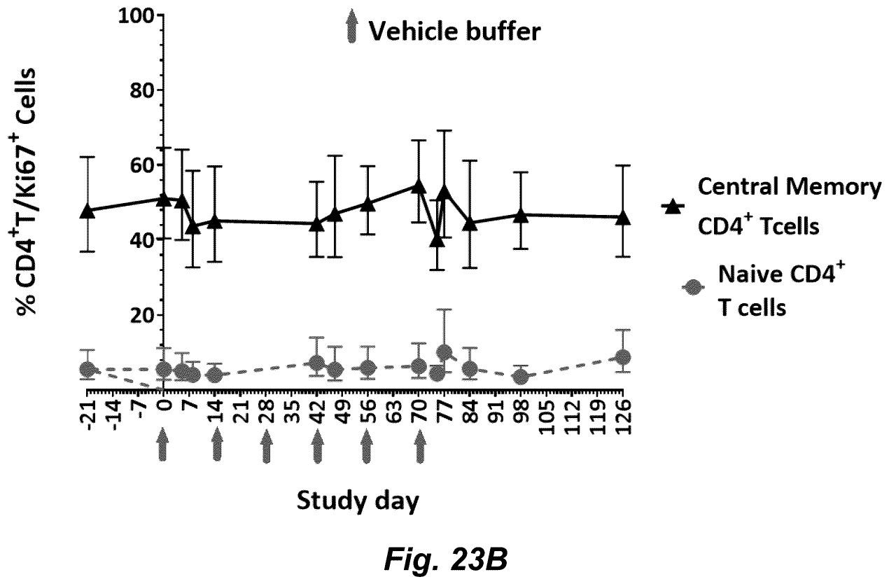

CD4+ T cell counts were strongly activated and peaked after 5-7 days, before declining prior to subsequent dosing (FIG. 22). We analyzed the CD4+ T cell subsets that were responding by staining for the proliferation marker Ki67. Previous work has indicated that untargeted IL-15 activates memory CD4+ T cells, but does not induce activation of naïve CD4+ T cells (Kanegane et al., Blood (1996) 88:230-235) and that the low IL-2Rβ expression on naïve CD4+ T cells constrains their ability to respond to IL-15 (Keller et al., J Immunol (2020) 204:3227-3235). This could be a concern for using IL-15 to activate HIV expression in reservoir cells, as naïve T cells have been found to harbor infectious HIV during ART suppression (Zerbato et al., Clin Inf Dis (2019) 69:1919-1925). The preferred method for HIV activation would be able to stimulate expression in all CD4+ T cell subsets. Surprisingly, CD4-targeted IL-15v was able to activate proliferation of both memory and naïve T cells, with the latter increasing from <10% Ki67+ cells prior to treatment to 80% Ki67+ cells at the peak after treatment (FIG. 23). This result was surprising to us because naïve CD4+ T cells usually do not respond to IL-15. This data in this example demonstrate that specifically targeting IL-15 to CD4+ T cells enhances the ability to activate naïve CD4+ T cells in addition to memory CD4+ T cells.

### Example 15

CD4-Targeted IL-15v with a Broadly Neutralizing Antibody Delays Viral Rebound in Chronically SHIV-Infected Rhesus Macaques

In this example, we evaluated the combination of a broadly neutralizing antibody, PGT-121, with a rhesus CD4-targeted IL-15v molecule in ART-suppressed rhesus macaques chronically infected with SHIV SF162P3 virus.

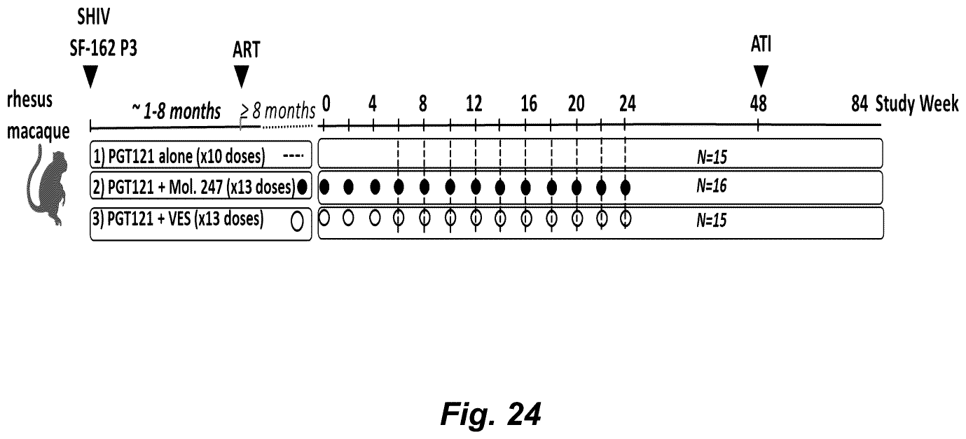

A combination efficacy study was conducted to determine if CD4-targeted IL15v could enhance bNAb-mediated reservoir reduction and post-ART viral control. Rhesus macaques were chronically infected with SHIV-SF162P3 for 1 to 8 months and then treated with ART for 8 to 24 months. Forty-six study animals were stratified into 3 groups: 10 mg/kg PGT-121 (N=15), 10 mg/kg PGT-121+0.5 mg/kg CD4-targeted IL-15v (Molecule 247) (N=16), or 10 mg/kg PGT-121+0.15 mg/kg vesatolimod (VES, N=15). PGT-121 and CD4-targeted IL-15v (Molecule 247) were dosed by IV administration and vesatolimod was dosed orally. The PGT-121+VES combination was included as a positive control, as previous studies indicated that this combination induced long term viral control in a similar model (Moldt et al., PLOS Path (2022) 18:e1010467; see also, Borducchi, et al., Nature (2018) 563(7731):360-364). Animals were distributed to balance several characteristics across each group, including baseline viral reservoir size in PBMCs as measured by intact proviral DNA assay (Bruner et al., Nature (2019) 566:120-125; Fray et al., Cell Host Microbe (2023) 31:1-17), pre-ART plasma set point viremia and body weight. Each group received 3 monotherapy doses every other week (vehicle alone in Group 1, CD4-targeted IL-15v in Group 2, VES in Group 3), followed by 10 doses where this dosing was continued in combination with PGT-121. After therapeutic dosing, PGT-121 was allowed to washout for 24 weeks. ART was interrupted at week 48. The study design is depicted in FIG. 24.

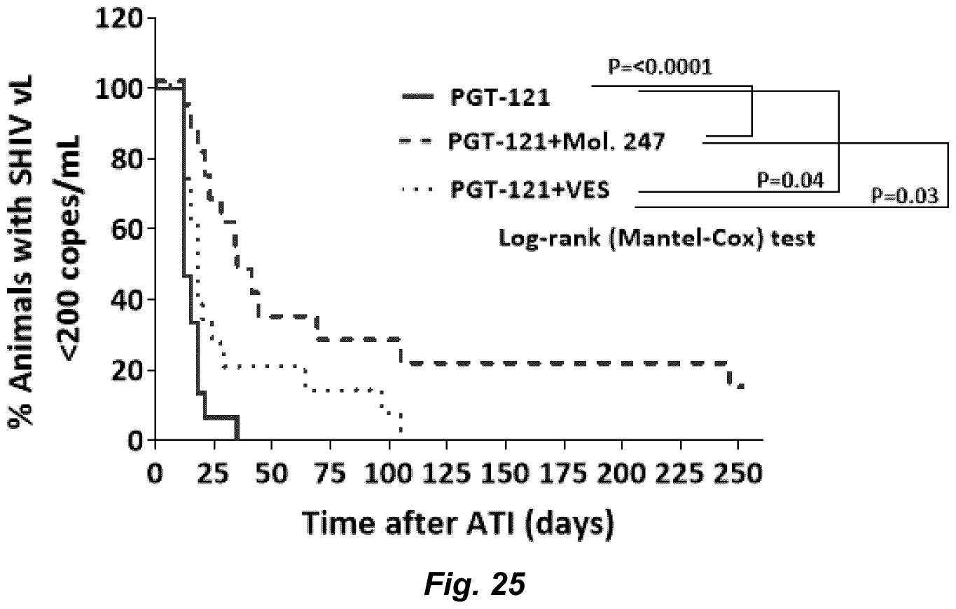

Viral rebound was monitored for up to 9 months after ATI (FIG. 25). Plasma SHIV viral loads were measured by SIV-Gag RNA specific RT-PCR assay. Rebound was defined as plasma viral load that exceeded 200 copies/ml. All animals in the PGT-121 group (15/15) rebounded within 35 days, with a median rebound time of 12 days. The median time to rebound in the PGT-121+VES group was 18 days, with 3 animals rebounding between 64 and 105 days. The largest and significant delay in rebound was seen in the PGT-121+CD4-targeted IL-15v (Molecule 247) group, with a median rebound of 35 days and 3 animals that did not rebound for over eight months. The results are depicted in FIG. 25. This data is consistent with the conclusion that CD4-targeted IL-15v can enhance viral control after treatment interruption when treated with a broadly neutralizing antibody in this model.

### Example 16

Combination Treatment with SMAC Mimetic and CD4-Targeted IL-15v in ART-Suppressed SIV-Infected Rhesus Macaques

SMAC mimetics (mimetics of the second mitochondrial-derived activator of caspases) have been shown to induce SIV expression during ART suppression through non canonical NF-κB signaling (Nixon et al., Nature (2020) 578:160-165). A combination of the SMAC mimetic AZD5582 with an untargeted IL-15 super agonist molecule, N-803 (a.k.a., ALT-803; nogapendekin alfa inbakicept), showed greater SIV activation than N-803 alone (Dashti et al., Nat Med (2023) 29:2535-2546), indicating possible synergy between IL-15 and SMAC mimetic signaling pathways involved in viral activation. Improved viral activation could enhance the clearance of persistent viral reservoirs in combination with effective reservoir targeting therapies.

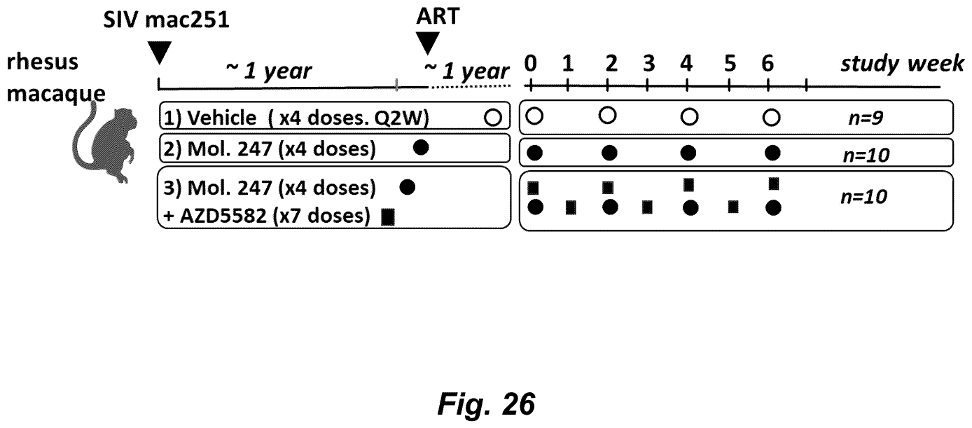

To evaluate the combination of CD4-targeted IL-15v and AZD5582 for viral activation, both molecules were co-dosed in SIV-infected rhesus macaques. Prior to dosing, rhesus macaques were chronically infected for 1 year with SIVmac251, then treated with ART for one year. Twenty-nine animals were distributed into three groups based on pre-ART viral load set points and body weights and were dosed as indicated in FIG. 26: (1) vehicle only (N=9), (2) 0.5 mg/kg Molecule 247 (IV) and (3) 0.5 mg/kg Molecule 247 with 0.1 mg/kg AZD5582 (both IV). Plasma SIV Gag RNA levels were measured by qRT-PCR (LLOQ: 3 copies/ml) throughout the dosing period to assess viral reactivation.

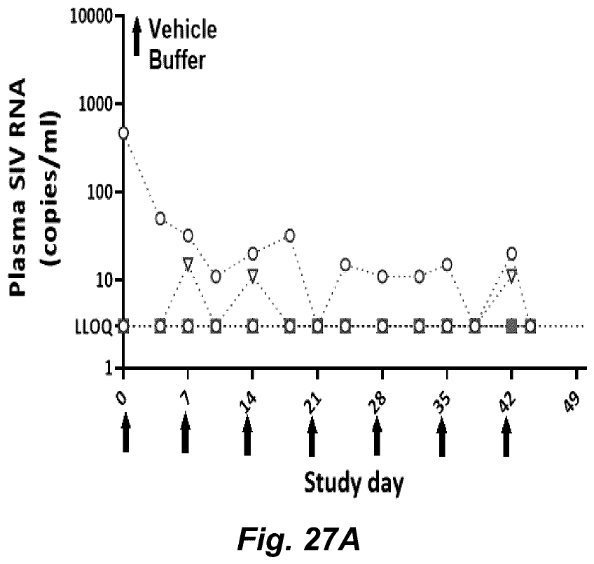

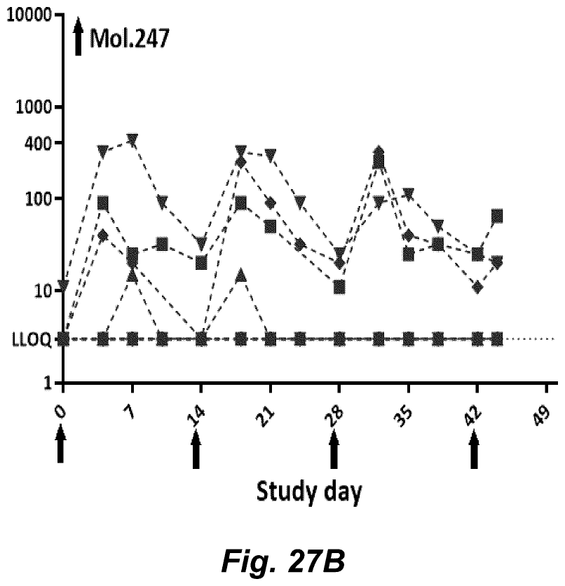

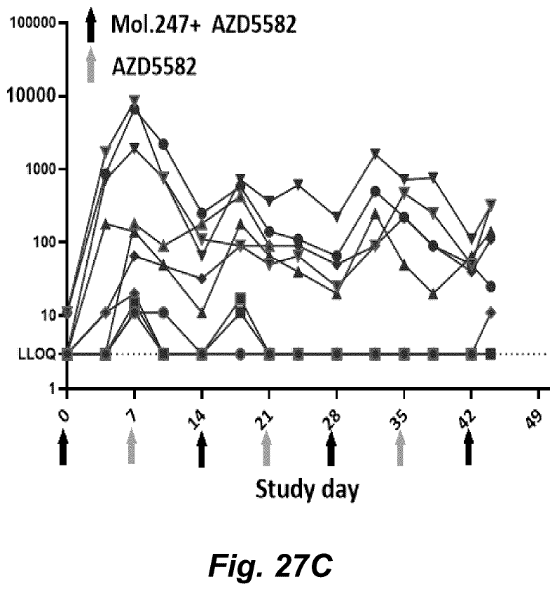

FIG. 27 shows the levels of plasma SIV RNA in each group. Occasional detectable plasma viral loads ≥3 copies/mL (the LLOQ) were observed in 1 to 2 animals in all three groups prior to the dosing period. Two of nine (2 of 9) animals in the vehicle group exhibited detectable viremia (greater than 3 copies/mL and higher than the baseline levels (if any) prior to dosing). Four of ten (4 of 10) animals in the Molecule 247 group exhibited detectable viremia by the same criteria and ten of ten (10 of 10) animals exhibited detectable viremia in the third group with Molecule 247 and AZD5582, indicating synergy between SMAC mimetic AZD5582 and Molecule 247. These data are consistent with the conclusion that AZD5582 enhanced the activation seen with CD4-targeted IL-15v in the SIV model.

### Example 17

### IL-15 Muteins Mediate Enhanced Tumor Cell Killing

In this example, we evaluated the ability of IL-15 muteins to mediate enhanced tumor cell killing, mainly by inducing proliferation, activation and degranulation of cytotoxic immune cells, shown here with NK cells.

### Methods

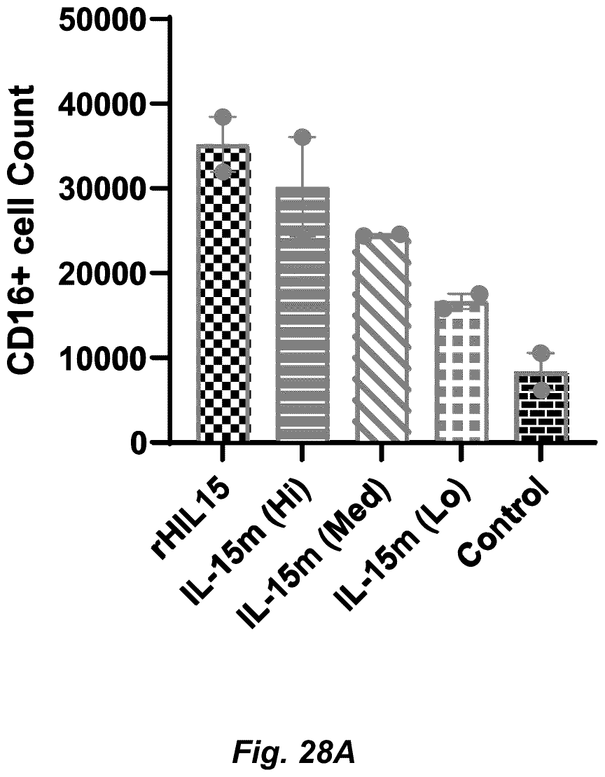

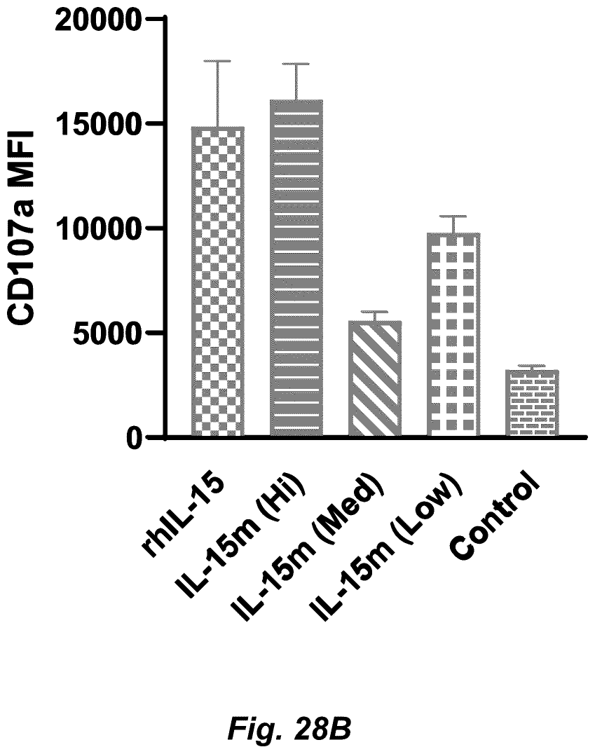

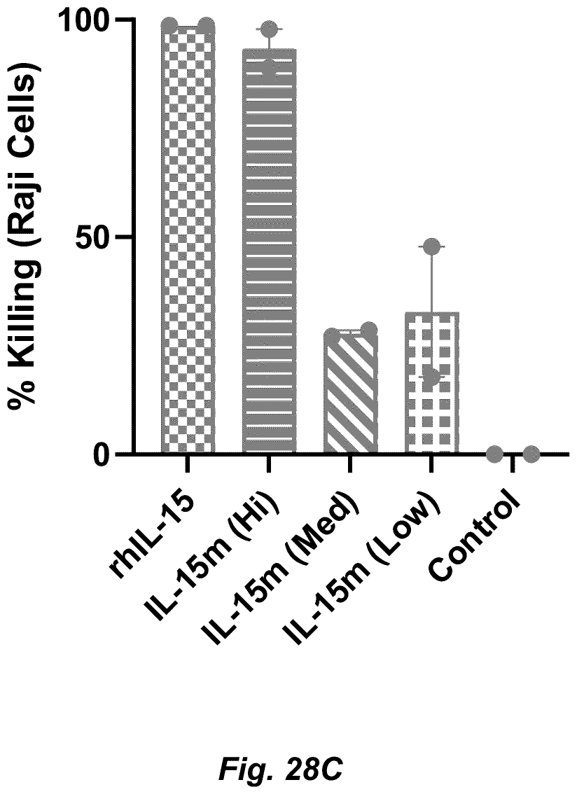

NK cells were isolated from PBMC using NK cell isolation kit (Stemcell Catalog #17955). Twenty thousand (20,000) cells were incubated with variable concentrations of Fc fusion proteins of IL-15 muteins: rhesus IL-15, IL-15v N65D (high affinity), IL-15v S7G.N65D (medium affinity), or IL-15v N65D_168A (low affinity) for 5 days. On day 5, twenty thousand (20,000) carboxyfluorescein succinimidyl ester (CFSE)-labeled Raji cells were added along with CD20-targeted NK cell engager (NKG2D×CD20) (1.1 nM) or control RSVF-targeted NK cell engager (NKG2D×RSV protein F (Palivizumab)) (1.1 nM) (FIG. 28A), or an antibody against CD107a (BioLegend Cat No 328638, 1:100 dilution) (FIG. 28B) or GolgiStop™ (BD Biosciences; Cat No. 554724) (FIG. 28C). Post 24 hours of co-culture, Live-Dead staining, counting beads, CD16 and CD19 staining were performed following standard protocol and run on a flow cytometry analyzer.

### Results

Fc fusion proteins of IL-15 muteins resulted in increased NK cell proliferation in an affinity-dependent manner, wherein the highest proliferation was observed when NK cells were cultured with high affinity IL-15v (N65D), compared to medium affinity IL-15v (S7G_N65D) and low affinity IL-15v (N65D_168A) (FIG. 28A). Recombinant human IL-15 was used as positive control. Furthermore, when these NK cells were incubated with CD20-targeted NK cell engager tool molecule, there was a significant increase in NK cell degranulation (FIG. 28B) and resulted in enhanced cytotoxicity of CD20-positive Raji cells (FIG. 28C). These results demonstrate the potential of using the herein described IL-15v to activate NK and T cells to mediate enhanced tumor cell killing, e.g., when incorporated into bispecific and multi-specific molecules.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An interleukin-15 variant (IL-15v) comprising the amino acid sequence of any one of SEQ ID NOs: 743, 750-752, 754-756, 758, 768-771, 774, 777, 779, 783-788, 790-798, 801-808, 810-813 and 1125.

2-3. (canceled)

4. A fusion protein comprising: (i) an IL-15 receptor alpha subunit (IL15RA) SUSHI domain and (ii) an IL-15 variant (IL-15v), the fusion protein comprising the amino acid sequence of any one of SEQ ID NOs: 817, 824-826, 828-830, 832, 842-845, 848, 851, 853, 857-862, 864-872, 875-882, 884-887 and 1127.

5-25. (canceled)

26. An antibody or antigen-binding fragment thereof that specifically binds to CD4 D3 comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences (according to Kabat) of:
1) SEQ ID NOs: 158, 159, 160, 145, 161 and 162; (1.20)
2) SEQ ID NOs: 163, 164, 160, 165, 166 and 162; (1.21)
3) SEQ ID NOs: 163, 164, 160, 165, 161 and 162; (1.22)
4) SEQ ID NOs: 158, 167, 168, 145, 98 and 169; (1.23)
5) SEQ ID NOs: 170, 171, 172, 173, 141 and 99; (2.1) or
6) SEQ ID NOs: 25, 174, 175, 176, 29 and 177; (2.8).

27-32. (canceled)

33. A CD4-targeted interleukin-15 (IL-15) molecule comprising:
a) a first polypeptide comprising an immunoglobulin heavy chain comprising a first immunoglobulin fragment crystallizable domain (Fc domain);
b) a second polypeptide comprising an immunoglobulin light chain (VL-CL), wherein the first polypeptide and the second polypeptide form an antigen binding domain that specifically binds to CD4; and
c) a third polypeptide comprising a fusion protein comprising: (i) an IL-15 receptor alpha subunit (IL15RA) SUSHI domain, (ii) an IL-15 or variant thereof (IL-15v), and (iii) a second Fc domain, wherein the first Fc domain and the second Fc domain heterodimerize to form a bispecific molecule that binds to CD4 and an IL-2βγ complex (CD122 and CD132).

34-54. (canceled)

55. The CD4-targeted IL-15 molecule of claim 33, wherein the antigen binding domain that specifically binds to CD4 specifically binds to the CD4 D3 domain.

56. The CD4-targeted IL-15 molecule of claim 55, wherein the antigen binding domain specifically binds to CD4 D3 and comprises a VH-complementarity determining region (CDR) 1; a VH-CDR2, a VH-CDR3, a VL-CDR1, a VL-CDR2, and a VL-CDR3 comprising, respectively, the amino acid sequences (according to Kabat) of:
1) SEQ ID NOs: 152, 153, 154, 155, 156 and 157; (OKT4)
2) SEQ ID NOs: 158, 159, 160, 145, 161 and 162; (1.20)
3) SEQ ID NOs: 163, 164, 160, 165, 166 and 162; (1.21)
4) SEQ ID NOs: 163, 164, 160, 165, 161 and 162; (1.22)
5) SEQ ID NOs: 158, 167, 168, 145, 98 and 169; (1.23)
6) SEQ ID NOs: 170, 171, 172, 173, 141 and 99; (2.1) or
7) SEQ ID NOs: 25, 174, 175, 176, 29 and 177; (2.8).

57-59. (canceled)

60. The CD4-targeted IL-15 molecule of any one of claims 55 to 59 claim 55, wherein the antigen binding domain that specifically binds to CD4 D3 and comprises a heavy chain variable region (VH) and a light chain variable region (VL) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 720 and 721; (OKT4)
2) SEQ ID NOs: 722 and 723; (1.20)
3) SEQ ID NOs: 724 and 725; (1.21)
4) SEQ ID NOs: 724 and 726; (1.22)
5) SEQ ID NOs: 727 and 728; (1.23)
6) SEQ ID NOs: 727 and 729; (1.23 VL C36Y)
7) SEQ ID NOs: 730 and 731; (2.1) or
8) SEQ ID NOs: 732 and 733; (2.8).

61. The CD4-targeted IL-15 molecule of claim 55, wherein the antibody or antigen-binding fragment thereof is insensitive to (i.e., binds to CD4 in the presence or absence of) the amino acid substitutions resulting from one or more of CD4 polymorphism variant IDs rs28919570 (R265W) and rs11064419 (F227S or F227C).

62-67. (canceled)

68. The CD4-targeted IL-15 molecule of claim 33, wherein the fusion protein comprises an IL-15 receptor alpha subunit (IL15RA) SUSHI domain that binds to IL-15, is no longer than 65 amino acids, and comprises the amino acid sequence of SEQ ID NO: 734, or an amino acid sequence at least 90%, 91%, 92%, 93%, 94%, 95%, 96%, 97%, 98%, 99% identical to SEQ ID NO: 734.

69-71. (canceled)

72. The CD4-targeted IL-15 molecule of claim 33, wherein the fusion protein comprises an IL-15 variant comprising one or more of the following amino acid substitutions:
S7G and N65D;
N65D and I68A;
S7G, N65D and I68A;
S7G, N65D, N71L, N79P and N112D;
S7G, N65D, N71E, N77L, N79P and N112D;
S7G, N65D, N71E, N77L, N79P and N112S;
S7G, N65D, N71L, N77L, N79P and N112K; or
S7G, N65D, N71E, N77L, N79P, N112K; wherein the position numbers are with respect to SEQ ID NO: 740.

73. The CD4-targeted IL-15 molecule of claim 33, comprising an IL-15 or IL-15v of any one of SEQ ID NOs: 740-813, 1124 and 1125.

74-82. (canceled)

83. The CD4-targeted IL-15 molecule of claim 33, comprising a fusion protein comprising: (i) an IL-15 receptor alpha subunit (IL15RA) SUSHI domain, (ii) an IL-15 or variant thereof (IL-15v) of any one of SEQ ID NOs: 1136, 1143-1145, 1147-1149, 1151, 1162-1165, 1168, 1171, 1173-1174, 1178-1183, 1185-1193 and 1196-1203, 1205-1208.

84-100. (canceled)

101. The CD4-targeted IL-15 molecule of claim 33, comprising a heavy chain (HC1) and a light chain (LC1) that bind to CD4 D3 and an IL15RASushi-IL15v-Fc fusion protein (HC2) comprising, respectively, the amino acid sequences set forth below, or amino acid sequences at least 90%, 91%, 92%, 93%, 94%, 95%, 96%, 97%, 98%, 99% identical to, respectively, the full length of the amino acid sequences set forth below:
1) SEQ ID NOs: 1089, 1090 and 1088; (246)
2) SEQ ID NOs: 1009, 1010 and 923; (165)
3) SEQ ID NOs: 1009, 1010 and 971; (166)
4) SEQ ID NOs: 1009, 1010 and 972; (167)
5) SEQ ID NOs: 1011, 1012 and 923; (168)
6) SEQ ID NOs: 1011, 1012 and 971; (169)
7) SEQ ID NOs: 1011, 1012 and 972; (170)
8) SEQ ID NOs: 1013, 1012 and 974; (171)
9) SEQ ID NOs: 1011, 1014 and 923; (172)
10) SEQ ID NOs: 1011, 1014 and 971; (173)
11) SEQ ID NOs: 1011, 1014 and 972; (174)
12) SEQ ID NOs: 1013, 1014 and 974; (175)
13) SEQ ID NOs: 1015, 1016 and 923; (176)
14) SEQ ID NOs: 1015, 1016 and 971; (177)
15) SEQ ID NOs: 1015, 1017 and 971; (178)
16) SEQ ID NOs: 1015, 1016 and 972; (179)
17) SEQ ID NOs: 1015, 1017 and 972; (180)
18) SEQ ID NOs: 1018, 1017 and 974; (181)
19) SEQ ID NOs: 1018, 1017 and 985; (182)
20) SEQ ID NOs: 1019, 1020 and 923; (183)
21) SEQ ID NOs: 1019, 1020 and 971; (184)
22) SEQ ID NOs: 1019, 1020 and 972; (185)
23) SEQ ID NOs: 1021, 1020 and 974; (186)
24) SEQ ID NOs: 1022, 1023 and 923; (187)
25) SEQ ID NOs: 1024, 1025 and 923; (188)
26) SEQ ID NOs: 1011, 1014 and 1026; (189)
27) SEQ ID NOs: 1027, 1014 and 1028; (190)
28) SEQ ID NOs: 1027, 1014 and 1029; (191)
29) SEQ ID NOs: 1011, 1014 and 1030; (192)
30) SEQ ID NOs: 1011, 1014 and 1031; (193)
31) SEQ ID NOs: 1011, 1014 and 1032; (194)
32) SEQ ID NOs: 1011, 1014 and 1077; (220)
33) SEQ ID NOs: 1011, 1014 and 1033; (195)
34) SEQ ID NOs: 1011, 1014 and 1034; (196)
35) SEQ ID NOs: 1011, 1014 and 1035; (197)
36) SEQ ID NOs: 1011, 1014 and 1036; (198)
37) SEQ ID NOs: 1011, 1014 and 1037; (199)
38) SEQ ID NOs: 1011, 1014 and 1038; (200)
39) SEQ ID NOs: 1011, 1014 and 1039; (201)
40) SEQ ID NOs: 1011, 1014 and 1040; (202)
41) SEQ ID NOs: 1011, 1014 and 1041; (203)
42) SEQ ID NOs: 1011, 1014 and 1042; (204)
43) SEQ ID NOs: 1011, 1014 and 1043; (205)
44) SEQ ID NOs: 1011, 1014 and 1044; (206)
45) SEQ ID NOs: 1011, 1014 and 1045; (207)
46) SEQ ID NOs: 1011, 1014 and 1046; (208)
47) SEQ ID NOs: 1011, 1014 and 1047; (209)
48) SEQ ID NOs: 1011, 1014 and 1048; (210)
49) SEQ ID NOs: 1011, 1014 and 1049; (211)
50) SEQ ID NOs: 1011, 1014 and 1050; (212)
51) SEQ ID NOs: 1011, 1014 and 1051; (213)
52) SEQ ID NOs: 1011, 1014 and 1052; (214)
53) SEQ ID NOs: 1011, 1014 and 1053; (215)
54) SEQ ID NOs: 1011, 1014 and 1054; (216)
55) SEQ ID NOs: 1011, 1014 and 1055; (217) or
56) SEQ ID NOs: 1011, 1014 and 1056; (218).

102-105. (canceled)

106. A polynucleotide or multiple polynucleotides encoding the CD4-targeted IL-15 molecule of claim 33.

107-118. (canceled)

119. A cell or population of cells, wherein the cell or population of cells expresses the CD4-targeted IL-15 molecule claim 33.

120-125. (canceled)

126. A method of producing a CD4-targeted IL-15 molecule, the method comprising:
a) culturing a cell or population of cells of claim 106, in a cell culture under conditions sufficient to express the CD4-targeted IL-15 molecules; and
b) isolating or purifying the CD4-targeted IL-15 molecules from the cell culture.

127-132. (canceled)

133. A pharmaceutical composition comprising the CD4-targeted IL-15 molecule of claim 33, and a pharmaceutically acceptable carrier.

134-171. (canceled)

172. A method of inducing, stimulating or promoting the proliferation of CD4+ T cells, comprising contacting the CD4+ T cells with an effective amount of the CD4-targeted IL-15 molecule of claim 33.

177. A method of activating a latent viral reservoir in a subject infected with human immunodeficiency virus (HIV), comprising administering to the subject a therapeutically effective amount of the CD4-targeted IL-15 molecule of claim 33.

178. A method of treating or preventing human immunodeficiency virus (HIV) in a subject in need thereof, comprising administering to the subject a therapeutically effective amount of the CD4-targeted IL-15 molecule of claim 33.

179-250. (canceled)

251. A kit comprising one or more unitary doses of a CD4-targeted IL-15 molecule of claim 33.

252-311. (canceled)


SEQUENCE LISTING (1230 sequences)

SEQ ID NO: 1 - protein, 5 aa, synthetic construct
NYAMS

SEQ ID NO: 2 - protein, 16 aa, synthetic construct
  VARIANT 4: note = A, D, E
  VARIANT 5: note = A, G, W, Y
  VARIANT 11: note = A, P
  VARIANT 16: note = G, S
SINXXGSTFYXDSVKX

SEQ ID NO: 3 - protein, 11 aa, synthetic construct
HYGGSYDPMDY

SEQ ID NO: 4 - protein, 11 aa, synthetic construct
  VARIANT 1: note = K, R
XASQDINKYIA

SEQ ID NO: 5 - protein, 7 aa, synthetic construct
  VARIANT 4: note = I, T
YTSXLHP

SEQ ID NO: 6 - protein, 9 aa, synthetic construct
  VARIANT 8: note = F, Q, Y
LQYDNPLXT

SEQ ID NO: 7 - protein, 16 aa, synthetic construct
  VARIANT 4: note = A, D, E
  VARIANT 5: note = A, G
  VARIANT 11: note = A, P
  VARIANT 16: note = G, S
SINXXGSTFYXDSVKX

SEQ ID NO: 8 - protein, 7 aa, synthetic construct
YTSTLHP

SEQ ID NO: 9 - protein, 9 aa, synthetic construct
LQYDNPLYT

SEQ ID NO: 10 - protein, 16 aa, synthetic construct
  VARIANT 5: note = W, Y
SINEXGSTFYADSVKS

SEQ ID NO: 11 - protein, 11 aa, synthetic construct
RASQDINKYIA

SEQ ID NO: 12 - protein, 7 aa, synthetic construct
YTSILHP

SEQ ID NO: 13 - protein, 16 aa, synthetic construct
  VARIANT 16: note = G, S
SINEGGSTFYADSVKX

SEQ ID NO: 14 - protein, 16 aa, synthetic construct
SINEGGSTFYADSVKS

SEQ ID NO: 15 - protein, 16 aa, synthetic construct
SINEWGSTFYADSVKS

SEQ ID NO: 16 - protein, 16 aa, synthetic construct
SINEYGSTFYADSVKS

SEQ ID NO: 17 - protein, 9 aa, synthetic construct
LQYDNPLQT

SEQ ID NO: 18 - protein, 9 aa, synthetic construct
LQYDNPLFT

SEQ ID NO: 19 - protein, 5 aa, synthetic construct
DYGMS

SEQ ID NO: 20 - protein, 17 aa, synthetic construct
GINWNGDSTGYADSVRG

SEQ ID NO: 21 - protein, 9 aa, synthetic construct
DGAIGGMDV

SEQ ID NO: 22 - protein, 11 aa, synthetic construct
RASQDIRDDLG

SEQ ID NO: 23 - protein, 7 aa, synthetic construct
SASTLQS

SEQ ID NO: 24 - protein, 9 aa, synthetic construct
LQDYNYPWT

SEQ ID NO: 25 - protein, 5 aa, synthetic construct
ELSMH

SEQ ID NO: 26 - protein, 17 aa, synthetic construct
YFDPRGGETIYAQKFQG

SEQ ID NO: 27 - protein, 13 aa, synthetic construct
GGDRDYYYYYMDV

SEQ ID NO: 28 - protein, 11 aa, synthetic construct
KASQDIDDDMN

SEQ ID NO: 29 - protein, 7 aa, synthetic construct
EATTLVP

SEQ ID NO: 30 - protein, 9 aa, synthetic construct
LQHDSFPYT

SEQ ID NO: 31 - protein, 5 aa, synthetic construct
KLSMH

SEQ ID NO: 32 - protein, 17 aa, synthetic construct
FFDPRDGERIYAQKFQG

SEQ ID NO: 33 - protein, 13 aa, synthetic construct
GGDYDYYYYYMGV

SEQ ID NO: 34 - protein, 11 aa, synthetic construct
KASQDIDDDLN

SEQ ID NO: 35 - protein, 9 aa, synthetic construct
LQHDNFPYT

SEQ ID NO: 36 - protein, 17 aa, synthetic construct
SFHPRDDEIIYAQKFQG

SEQ ID NO: 37 - protein, 13 aa, synthetic construct
GGNPDYYFYYMDV

SEQ ID NO: 38 - protein, 9 aa, synthetic construct
LQHDNFPLT

SEQ ID NO: 39 - protein, 13 aa, synthetic construct
GGNKDYYFYYMDV

SEQ ID NO: 40 - protein, 5 aa, synthetic construct
ALSMH

SEQ ID NO: 41 - protein, 17 aa, synthetic construct
YFNPRDGETIYAQKFQG

SEQ ID NO: 42 - protein, 13 aa, synthetic construct
GGDSDYYYYYMDV

SEQ ID NO: 43 - protein, 17 aa, synthetic construct
YFNPRDDETIYTQKFQG

SEQ ID NO: 44 - protein, 17 aa, synthetic construct
VFDPKYGETTYAQKFQD

SEQ ID NO: 45 - protein, 13 aa, synthetic construct
GGNYEYFYYYMDV

SEQ ID NO: 46 - protein, 17 aa, synthetic construct
IFDPKYGETMYAQKFQG

SEQ ID NO: 47 - protein, 13 aa, synthetic construct
GGSPDYFYYYMDV

SEQ ID NO: 48 - protein, 17 aa, synthetic construct
IFDPRNGETIYAQKFQG

SEQ ID NO: 49 - protein, 13 aa, synthetic construct
GGSPDYYYWYMDV

SEQ ID NO: 50 - protein, 13 aa, synthetic construct
GGSPDYYFWYMDV

SEQ ID NO: 51 - protein, 5 aa, synthetic construct
SYDIN

SEQ ID NO: 52 - protein, 17 aa, synthetic construct
WMNPNSGNTGSTQKFQG

SEQ ID NO: 53 - protein, 12 aa, synthetic construct
GVLKGDYYYMDV

SEQ ID NO: 54 - protein, 12 aa, synthetic construct
RASQSISSSYLS

SEQ ID NO: 55 - protein, 7 aa, synthetic construct
GSSTRAT

SEQ ID NO: 56 - protein, 9 aa, synthetic construct
QQDDHLPLT

SEQ ID NO: 57 - protein, 17 aa, synthetic construct
WVNPNSGNTGSTQKFQG

SEQ ID NO: 58 - protein, 12 aa, synthetic construct
RASQSISSTYLS

SEQ ID NO: 59 - protein, 6 aa, synthetic construct
GDYYWF

SEQ ID NO: 60 - protein, 17 aa, synthetic construct
YIYGSGGGTNYNPSLNN

SEQ ID NO: 61 - protein, 11 aa, synthetic construct
NILKYLHWLLY

SEQ ID NO: 62 - protein, 11 aa, synthetic construct
GGDNVGRKSVQ

SEQ ID NO: 63 - protein, 7 aa, synthetic construct
ADSERPS

SEQ ID NO: 64 - protein, 11 aa, synthetic construct
QVWDSTADHWV

SEQ ID NO: 65 - protein, 5 aa, synthetic construct
DYVIH

SEQ ID NO: 66 - protein, 17 aa, synthetic construct
EIYPGSGSAYSNAKFKD

SEQ ID NO: 67 - protein, 9 aa, synthetic construct
RGNGTGFAY

SEQ ID NO: 68 - protein, 15 aa, synthetic construct
KAGQSVDYDGDSYMN

SEQ ID NO: 69 - protein, 7 aa, synthetic construct
VASNLES

SEQ ID NO: 70 - protein, 9 aa, synthetic construct
QQSYKDPLT

SEQ ID NO: 71 - protein, 5 aa, synthetic construct
SYVIH

SEQ ID NO: 72 - protein, 17 aa, synthetic construct
YINPYNDGTDYDEKFKG

SEQ ID NO: 73 - protein, 13 aa, synthetic construct
EKDNYATGAWFAY

SEQ ID NO: 74 - protein, 17 aa, synthetic construct
KSSQSLLYSTNQKNYLA

SEQ ID NO: 75 - protein, 7 aa, synthetic construct
WASTRES

SEQ ID NO: 76 - protein, 8 aa, synthetic construct
QQYYSYRT

SEQ ID NO: 77 - protein, 5 aa, synthetic construct
DCRMY

SEQ ID NO: 78 - protein, 19 aa, synthetic construct
VISVKSENYGANYAESVRG

SEQ ID NO: 79 - protein, 13 aa, synthetic construct
SYYRYDVGAWFAY

SEQ ID NO: 80 - protein, 15 aa, synthetic construct
RASKSVSTSGYSYIY

SEQ ID NO: 81 - protein, 7 aa, synthetic construct
LASILES

SEQ ID NO: 82 - protein, 9 aa, synthetic construct
QHSRELPWT

SEQ ID NO: 83 - protein, 17 aa, synthetic construct
GFDPEDGKTIYAPKFQG

SEQ ID NO: 84 - protein, 16 aa, synthetic construct
GHNWNDGYYFYYYMDL

SEQ ID NO: 85 - protein, 16 aa, synthetic construct
KSSQSLLHTDGKTYLY

SEQ ID NO: 86 - protein, 7 aa, synthetic construct
EVSNRFS

SEQ ID NO: 87 - protein, 9 aa, synthetic construct
MQSLQLPLT

SEQ ID NO: 88 - protein, 5 aa, synthetic construct
DYYMN

SEQ ID NO: 89 - protein, 17 aa, synthetic construct
YISSSGNTIFYVDSVEG

SEQ ID NO: 90 - protein, 16 aa, synthetic construct
EGYNNYNYSYYYFMDV

SEQ ID NO: 91 - protein, 11 aa, synthetic construct
RASQSVSRKLA

SEQ ID NO: 92 - protein, 7 aa, synthetic construct
AASTRAT

SEQ ID NO: 93 - protein, 9 aa, synthetic construct
QQYNNWPYT

SEQ ID NO: 94 - protein, 5 aa, synthetic construct
SFGMH

SEQ ID NO: 95 - protein, 17 aa, synthetic construct
IIWYDGTNKYYADSVKG

SEQ ID NO: 96 - protein, 13 aa, synthetic construct
EIAVDGTDYYMDV

SEQ ID NO: 97 - protein, 11 aa, synthetic construct
RASQGIRSGLG

SEQ ID NO: 98 - protein, 7 aa, synthetic construct
AASTLQS

SEQ ID NO: 99 - protein, 9 aa, synthetic construct
LQDYNYPYT

SEQ ID NO: 100 - protein, 17 aa, synthetic construct
YISSSGLTIFYVDSVKG

SEQ ID NO: 101 - protein, 16 aa, synthetic construct
EGYSGFDDHYYYYTDV

SEQ ID NO: 102 - protein, 11 aa, synthetic construct
RASQSVSSNLA

SEQ ID NO: 103 - protein, 7 aa, synthetic construct
GASIRAT

SEQ ID NO: 104 - protein, 9 aa, synthetic construct
QHYKNWPYT

SEQ ID NO: 105 - protein, 17 aa, synthetic construct
YISSSGSTIFYTDSVKG

SEQ ID NO: 106 - protein, 16 aa, synthetic construct
EDYSNYEDYYYYYTGV

SEQ ID NO: 107 - protein, 11 aa, synthetic construct
RASQSVSSKFA

SEQ ID NO: 108 - protein, 7 aa, synthetic construct
NASTRAT

SEQ ID NO: 109 - protein, 17 aa, synthetic construct
YISSSGITIFYTDSVKG

SEQ ID NO: 110 - protein, 16 aa, synthetic construct
EDYSNYEDHYYYYTGV

SEQ ID NO: 111 - protein, 11 aa, synthetic construct
RASQSVSNKFA

SEQ ID NO: 112 - protein, 9 aa, synthetic construct
QHYNNWPYT

SEQ ID NO: 113 - protein, 7 aa, synthetic construct
GASTRAT

SEQ ID NO: 114 - protein, 17 aa, synthetic construct
YISSSGKTIFYTDSVKG

SEQ ID NO: 115 - protein, 9 aa, synthetic construct
HQYKNWPYT

SEQ ID NO: 116 - protein, 17 aa, synthetic construct
YISISGQTIYYGDSVKG

SEQ ID NO: 117 - protein, 16 aa, synthetic construct
EGYSNYGVKYYYYMDV

SEQ ID NO: 118 - protein, 11 aa, synthetic construct
RASQSISSNLA

SEQ ID NO: 119 - protein, 9 aa, synthetic construct
QQYNAWTYT

SEQ ID NO: 120 - protein, 5 aa, synthetic construct
DHYMN

SEQ ID NO: 121 - protein, 17 aa, synthetic construct
YISISGNTIYYTDSVKG

SEQ ID NO: 122 - protein, 17 aa, synthetic construct
EGYSSSSRGDYSYYTDV

SEQ ID NO: 123 - protein, 9 aa, synthetic construct
QQYKNWPYT

SEQ ID NO: 124 - protein, 17 aa, synthetic construct
YISISGRTIYYIDSVKG

SEQ ID NO: 125 - protein, 11 aa, synthetic construct
RASQGVSSNLA

SEQ ID NO: 126 - protein, 5 aa, synthetic construct
SYGMH

SEQ ID NO: 127 - protein, 17 aa, synthetic construct
IIWFDGSNKYYADSLTG

SEQ ID NO: 128 - protein, 13 aa, synthetic construct
EVALEGYYYYMDV

SEQ ID NO: 129 - protein, 11 aa, synthetic construct
RASQDIRNGLG

SEQ ID NO: 130 - protein, 7 aa, synthetic construct
TTSSLQT

SEQ ID NO: 131 - protein, 17 aa, synthetic construct
ILWFDGSNKFYADSVKG

SEQ ID NO: 132 - protein, 13 aa, synthetic construct
ELALEGYYYYMDV

SEQ ID NO: 133 - protein, 7 aa, synthetic construct
TTSSLQS

SEQ ID NO: 134 - protein, 17 aa, synthetic construct
ILWFDGSNQFYADSVKG

SEQ ID NO: 135 - protein, 13 aa, synthetic construct
EIALEGYYYYMDV

SEQ ID NO: 136 - protein, 11 aa, synthetic construct
RSSQDIRNGLG

SEQ ID NO: 137 - protein, 17 aa, synthetic construct
IIWYDGSNKYYADSVKG

SEQ ID NO: 138 - protein, 7 aa, synthetic construct
TTSNLQS

SEQ ID NO: 139 - protein, 17 aa, synthetic construct
IIWYDGNNKYYADSVKG

SEQ ID NO: 140 - protein, 11 aa, synthetic construct
RASQGIRNGLG

SEQ ID NO: 141 - protein, 7 aa, synthetic construct
AASSLQS

SEQ ID NO: 142 - protein, 5 aa, synthetic construct
SYAMS

SEQ ID NO: 143 - protein, 17 aa, synthetic construct
GISGSVGSTYYADSVKG

SEQ ID NO: 144 - protein, 10 aa, synthetic construct
EGYDWSGMDV

SEQ ID NO: 145 - protein, 11 aa, synthetic construct
RASQGIRNDLG

SEQ ID NO: 146 - protein, 17 aa, synthetic construct
LIWFDGTTRFYADSVKG

SEQ ID NO: 147 - protein, 13 aa, synthetic construct
EVAIQGRDYYIDV

SEQ ID NO: 148 - protein, 5 aa, synthetic construct
SYGIH

SEQ ID NO: 149 - protein, 17 aa, synthetic construct
IIWFDGTNKFYADSVKG

SEQ ID NO: 150 - protein, 13 aa, synthetic construct
EVAIQGYYYYMDV

SEQ ID NO: 151 - protein, 7 aa, synthetic construct
TTSNLQN

SEQ ID NO: 152 - protein, 5 aa, synthetic construct
NYGMN

SEQ ID NO: 153 - protein, 17 aa, synthetic construct
WINTNTGEPTYAEEFKG

SEQ ID NO: 154 - protein, 14 aa, synthetic construct
LGIYYDYGYYAMDY

SEQ ID NO: 155 - protein, 15 aa, synthetic construct
RASESVDSYGNSFMH

SEQ ID NO: 156 - protein, 7 aa, synthetic construct
LASNLES

SEQ ID NO: 157 - protein, 9 aa, synthetic construct
QQNNEDPYT

SEQ ID NO: 158 - protein, 5 aa, synthetic construct
TYAMS

SEQ ID NO: 159 - protein, 17 aa, synthetic construct
GISGSGDNTYYADSVKG

SEQ ID NO: 160 - protein, 10 aa, synthetic construct
EGYNWNYMDV

SEQ ID NO: 161 - protein, 7 aa, synthetic construct
EASSLQS

SEQ ID NO: 162 - protein, 9 aa, synthetic construct
LQDYTYPYT

SEQ ID NO: 163 - protein, 5 aa, synthetic construct
TFAMS

SEQ ID NO: 164 - protein, 17 aa, synthetic construct
GISGSGENTYYADSVKG

SEQ ID NO: 165 - protein, 11 aa, synthetic construct
RASHGIRNDLG

SEQ ID NO: 166 - protein, 7 aa, synthetic construct
ETSSLQS

SEQ ID NO: 167 - protein, 17 aa, synthetic construct
TISDNIGNTYYADSVKG

SEQ ID NO: 168 - protein, 9 aa, synthetic construct
DNEDYYMDV

SEQ ID NO: 169 - protein, 9 aa, synthetic construct
LQDYNYPRT

SEQ ID NO: 170 - protein, 5 aa, synthetic construct
TNAMS

SEQ ID NO: 171 - protein, 17 aa, synthetic construct
GISGSNGNTYYADSVKG

SEQ ID NO: 172 - protein, 10 aa, synthetic construct
ERDNWNGFDY

SEQ ID NO: 173 - protein, 11 aa, synthetic construct
RASHVIRNDLG

SEQ ID NO: 174 - protein, 17 aa, synthetic construct
IFDPRDGQIIYAEKFQG

SEQ ID NO: 175 - protein, 13 aa, synthetic construct
GGNEDYYFYYMDV

SEQ ID NO: 176 - protein, 11 aa, synthetic construct
KASQDIDDDVN

SEQ ID NO: 177 - protein, 9 aa, synthetic construct
LQHDHFPYT

SEQ ID NO: 178 - protein, 8 aa, synthetic construct
GFSFSNYA

SEQ ID NO: 179 - protein, 7 aa, synthetic construct
  VARIANT 3: note = A, D, E
  VARIANT 4: note = A, G, W, Y
INXXGST

SEQ ID NO: 180 - protein, 13 aa, synthetic construct
SRHYGGSYDPMDY

SEQ ID NO: 181 - protein, 6 aa, synthetic construct
QDINKY

SEQ ID NO: 182 - skipped

SEQ ID NO: 183 - protein, 7 aa, synthetic construct
  VARIANT 3: note = A, D, E
  VARIANT 4: note = A, G
INXXGST

SEQ ID NO: 184 - protein, 7 aa, synthetic construct
  VARIANT 4: note = W, Y
INEXGST

SEQ ID NO: 185 - protein, 7 aa, synthetic construct
INEGGST

SEQ ID NO: 186 - protein, 7 aa, synthetic construct
INEWGST

SEQ ID NO: 187 - protein, 7 aa, synthetic construct
INEYGST

SEQ ID NO: 188 - protein, 7 aa, synthetic construct
INDGGST

SEQ ID NO: 189 - protein, 7 aa, synthetic construct
INAGGST

SEQ ID NO: 190 - protein, 7 aa, synthetic construct
INDAGST

SEQ ID NO: 191 - protein, 8 aa, synthetic construct
GFTFDDYG

SEQ ID NO: 192 - protein, 8 aa, synthetic construct
INWNGDST

SEQ ID NO: 193 - protein, 11 aa, synthetic construct
ARDGAIGGMDV

SEQ ID NO: 194 - protein, 6 aa, synthetic construct
QDIRDD

SEQ ID NO: 195 - skipped

SEQ ID NO: 196 - protein, 8 aa, synthetic construct
GHTLTELS

SEQ ID NO: 197 - protein, 8 aa, synthetic construct
FDPRGGET

SEQ ID NO: 198 - protein, 15 aa, synthetic construct
ATGGDRDYYYYYMDV

SEQ ID NO: 199 - protein, 6 aa, synthetic construct
QDIDDD

SEQ ID NO: 200 - skipped

SEQ ID NO: 201 - protein, 8 aa, synthetic construct
GFTLTKLS

SEQ ID NO: 202 - protein, 8 aa, synthetic construct
FDPRDGER

SEQ ID NO: 203 - protein, 15 aa, synthetic construct
ATGGDYDYYYYYMGV

SEQ ID NO: 204 - protein, 8 aa, synthetic construct
GHTVTELS

SEQ ID NO: 205 - protein, 8 aa, synthetic construct
FHPRDDEI

SEQ ID NO: 206 - protein, 15 aa, synthetic construct
ATGGNPDYYFYYMDV

SEQ ID NO: 207 - protein, 8 aa, synthetic construct
FHPRDGET

SEQ ID NO: 208 - protein, 15 aa, synthetic construct
ATGGNKDYYFYYMDV

SEQ ID NO: 209 - protein, 8 aa, synthetic construct
GYTLTALS

SEQ ID NO: 210 - protein, 8 aa, synthetic construct
FNPRDGET

SEQ ID NO: 211 - protein, 15 aa, synthetic construct
ATGGDSDYYYYYMDV

SEQ ID NO: 212 - protein, 8 aa, synthetic construct
FNPRDDET

SEQ ID NO: 213 - protein, 8 aa, synthetic construct
FDPKYGET

SEQ ID NO: 214 - protein, 15 aa, synthetic construct
ATGGNYEYFYYYMDV

SEQ ID NO: 215 - protein, 15 aa, synthetic construct
ATGGSPDYFYYYMDV

SEQ ID NO: 216 - protein, 8 aa, synthetic construct
FDPRNGET

SEQ ID NO: 217 - protein, 15 aa, synthetic construct
ATGGSPDYYYWYMDV

SEQ ID NO: 218 - protein, 15 aa, synthetic construct
ATGGSPDYYFWYMDV

SEQ ID NO: 219 - protein, 8 aa, synthetic construct
GYTFTSYD

SEQ ID NO: 220 - protein, 8 aa, synthetic construct
MNPNSGNT

SEQ ID NO: 221 - protein, 14 aa, synthetic construct
ARGVLKGDYYYMDV

SEQ ID NO: 222 - protein, 7 aa, synthetic construct
QSISSSY

SEQ ID NO: 223 - skipped

SEQ ID NO: 224 - protein, 8 aa, synthetic construct
VNPNSGNT

SEQ ID NO: 225 - protein, 7 aa, synthetic construct
QSISSTY

SEQ ID NO: 226 - protein, 9 aa, synthetic construct
GGSISGDYY

SEQ ID NO: 227 - protein, 8 aa, synthetic construct
IYGSGGGT

SEQ ID NO: 228 - protein, 13 aa, synthetic construct
ASNILKYLHWLLY

SEQ ID NO: 229 - protein, 6 aa, synthetic construct
NVGRKS

SEQ ID NO: 230 - skipped

SEQ ID NO: 231 - protein, 8 aa, synthetic construct
GYTFTDYV

SEQ ID NO: 232 - protein, 8 aa, synthetic construct
IYPGSGSA

SEQ ID NO: 233 - protein, 11 aa, synthetic construct
ARRGNGTGFAY

SEQ ID NO: 234 - protein, 10 aa, synthetic construct
QSVDYDGDSY

SEQ ID NO: 235 - skipped

SEQ ID NO: 236 - protein, 8 aa, synthetic construct
GYTFTSYV

SEQ ID NO: 237 - protein, 8 aa, synthetic construct
INPYNDGT

SEQ ID NO: 238 - protein, 15 aa, synthetic construct
AREKDNYATGAWFAY

SEQ ID NO: 239 - protein, 12 aa, synthetic construct
QSLLYSTNQKNY

SEQ ID NO: 240 - skipped

SEQ ID NO: 241 - protein, 8 aa, synthetic construct
GFSFSDCR

SEQ ID NO: 242 - protein, 10 aa, synthetic construct
ISVKSENYGA

SEQ ID NO: 243 - protein, 15 aa, synthetic construct
SASYYRYDVGAWFAY

SEQ ID NO: 244 - protein, 10 aa, synthetic construct
KSVSTSGYSY

SEQ ID NO: 245 - skipped

SEQ ID NO: 246 - protein, 8 aa, synthetic construct
GYTLTELS

SEQ ID NO: 247 - protein, 8 aa, synthetic construct
FDPEDGKT

SEQ ID NO: 248 - protein, 18 aa, synthetic construct
ATGHNWNDGYYFYYYMDL

SEQ ID NO: 249 - protein, 11 aa, synthetic construct
QSLLHTDGKTY

SEQ ID NO: 250 - skipped

SEQ ID NO: 251 - protein, 8 aa, synthetic construct
GFTFSDYY

SEQ ID NO: 252 - protein, 8 aa, synthetic construct
ISSSGNTI

SEQ ID NO: 253 - protein, 18 aa, synthetic construct
AREGYNNYNYSYYYFMDV

SEQ ID NO: 254 - protein, 6 aa, synthetic construct
QSVSRK

SEQ ID NO: 255 - skipped

SEQ ID NO: 256 - protein, 8 aa, synthetic construct
GFTVSSFG

SEQ ID NO: 257 - protein, 8 aa, synthetic construct
IWYDGTNK

SEQ ID NO: 258 - protein, 15 aa, synthetic construct
AREIAVDGTDYYMDV

SEQ ID NO: 259 - protein, 6 aa, synthetic construct
QGIRSG

SEQ ID NO: 260 - protein, 8 aa, synthetic construct
ISSSGLTI

SEQ ID NO: 261 - protein, 18 aa, synthetic construct
AREGYSGFDDHYYYYTDV

SEQ ID NO: 262 - protein, 6 aa, synthetic construct
QSVSSN

SEQ ID NO: 263 - skipped

SEQ ID NO: 264 - protein, 8 aa, synthetic construct
ISSSGSTI

SEQ ID NO: 265 - protein, 18 aa, synthetic construct
AREDYSNYEDYYYYYTGV

SEQ ID NO: 266 - protein, 6 aa, synthetic construct
QSVSSK

SEQ ID NO: 267 - skipped

SEQ ID NO: 268 - protein, 8 aa, synthetic construct
ISSSGITI

SEQ ID NO: 269 - protein, 18 aa, synthetic construct
AREDYSNYEDHYYYYTGV

SEQ ID NO: 270 - protein, 6 aa, synthetic construct
QSVSNK

SEQ ID NO: 271 - protein, 8 aa, synthetic construct
ISSSGKTI

SEQ ID NO: 272 - protein, 8 aa, synthetic construct
ISISGQTI

SEQ ID NO: 273 - protein, 18 aa, synthetic construct
AREGYSNYGVKYYYYMDV

SEQ ID NO: 274 - protein, 6 aa, synthetic construct
QSISSN

SEQ ID NO: 275 - protein, 8 aa, synthetic construct
GFTFSDHY

SEQ ID NO: 276 - protein, 8 aa, synthetic construct
ISISGNTI

SEQ ID NO: 277 - protein, 19 aa, synthetic construct
VREGYSSSSRGDYSYYTDV

SEQ ID NO: 278 - protein, 8 aa, synthetic construct
ISISGRTI

SEQ ID NO: 279 - protein, 6 aa, synthetic construct
QGVSSN

SEQ ID NO: 280 - protein, 8 aa, synthetic construct
GFTVSSYG

SEQ ID NO: 281 - protein, 8 aa, synthetic construct
IWFDGSNK

SEQ ID NO: 282 - protein, 15 aa, synthetic construct
AREVALEGYYYYMDV

SEQ ID NO: 283 - protein, 6 aa, synthetic construct
QDIRNG

SEQ ID NO: 284 - skipped

SEQ ID NO: 285 - protein, 8 aa, synthetic construct
LWFDGSNK

SEQ ID NO: 286 - protein, 15 aa, synthetic construct
ARELALEGYYYYMDV

SEQ ID NO: 287 - protein, 8 aa, synthetic construct
LWFDGSNQ

SEQ ID NO: 288 - protein, 15 aa, synthetic construct
AREIALEGYYYYMDV

SEQ ID NO: 289 - protein, 8 aa, synthetic construct
GFTFSSYG

SEQ ID NO: 290 - protein, 8 aa, synthetic construct
IWYDGSNK

SEQ ID NO: 291 - protein, 8 aa, synthetic construct
IWYDGNNK

SEQ ID NO: 292 - protein, 15 aa, synthetic construct
TREVALEGYYYYMDV

SEQ ID NO: 293 - protein, 6 aa, synthetic construct
QGIRNG

SEQ ID NO: 294 - protein, 8 aa, synthetic construct
GFTFSSYA

SEQ ID NO: 295 - protein, 8 aa, synthetic construct
ISGSVGST

SEQ ID NO: 296 - protein, 12 aa, synthetic construct
AKEGYDWSGMDV

SEQ ID NO: 297 - protein, 6 aa, synthetic construct
QGIRND

SEQ ID NO: 298 - protein, 8 aa, synthetic construct
IWFDGTTR

SEQ ID NO: 299 - protein, 15 aa, synthetic construct
AREVAIQGRDYYIDV

SEQ ID NO: 300 - protein, 8 aa, synthetic construct
IWFDGTNK

SEQ ID NO: 301 - protein, 15 aa, synthetic construct
AREVAIQGYYYYMDV

SEQ ID NO: 302 - protein, 8 aa, synthetic construct
GYTFTNYG

SEQ ID NO: 303 - protein, 8 aa, synthetic construct
INTNTGEP

SEQ ID NO: 304 - protein, 16 aa, synthetic construct
ARLGIYYDYGYYAMDY

SEQ ID NO: 305 - protein, 10 aa, synthetic construct
ESVDSYGNSF

SEQ ID NO: 306 - protein, 8 aa, synthetic construct
EIIFSTYA

SEQ ID NO: 307 - protein, 8 aa, synthetic construct
ISGSGDNT

SEQ ID NO: 308 - skipped

SEQ ID NO: 309 - protein, 8 aa, synthetic construct
GLTFSTFA

SEQ ID NO: 310 - protein, 8 aa, synthetic construct
ISGSGENT

SEQ ID NO: 311 - protein, 12 aa, synthetic construct
AKEGYNWNYMDV

SEQ ID NO: 312 - protein, 6 aa, synthetic construct
HGIRND

SEQ ID NO: 313 - skipped

SEQ ID NO: 314 - protein, 8 aa, synthetic construct
GFTFSTYA

SEQ ID NO: 315 - protein, 8 aa, synthetic construct
ISDNIGNT

SEQ ID NO: 316 - protein, 11 aa, synthetic construct
TKDNEDYYMDV

SEQ ID NO: 317 - protein, 8 aa, synthetic construct
GFTFSTNA

SEQ ID NO: 318 - protein, 8 aa, synthetic construct
ISGSNGNT

SEQ ID NO: 319 - protein, 12 aa, synthetic construct
AKERDNWNGFDY

SEQ ID NO: 320 - protein, 6 aa, synthetic construct
HVIRND

SEQ ID NO: 321 - protein, 8 aa, synthetic construct
FDPRDGQI

SEQ ID NO: 322 - protein, 15 aa, synthetic construct
ATGGNEDYYFYYMDV

SEQ ID NO: 323 - protein, 7 aa, synthetic construct
GFSFSNY

SEQ ID NO: 324 - skipped

SEQ ID NO: 325 - protein, 9 aa, synthetic construct
YGGSYDPMD

SEQ ID NO: 326 - protein, 7 aa, synthetic construct
SQDINKY

SEQ ID NO: 327 - protein, 6 aa, synthetic construct
  VARIANT 6: note = F, Q, Y
YDNPLX

SEQ ID NO: 328 - skipped

SEQ ID NO: 329 - protein, 6 aa, synthetic construct
YDNPLY

SEQ ID NO: 330-333 - skipped (4 entries)

SEQ ID NO: 334 - protein, 6 aa, synthetic construct
YDNPLQ

SEQ ID NO: 335 - protein, 6 aa, synthetic construct
YDNPLF

SEQ ID NO: 336-338 - skipped (3 entries)

SEQ ID NO: 339 - protein, 7 aa, synthetic construct
GFTFDDY

SEQ ID NO: 340 - protein, 4 aa, synthetic construct
WNGD

SEQ ID NO: 341 - protein, 7 aa, synthetic construct
GAIGGMD

SEQ ID NO: 342 - protein, 7 aa, synthetic construct
SQDIRDD

SEQ ID NO: 343 - protein, 7 aa, synthetic construct
GHTLTEL

SEQ ID NO: 344 - protein, 7 aa, synthetic construct
GHTLTEL

SEQ ID NO: 345 - protein, 4 aa, synthetic construct
PRGG

SEQ ID NO: 346 - protein, 11 aa, synthetic construct
GDRDYYYYYMD

SEQ ID NO: 347 - protein, 7 aa, synthetic construct
SQDIDDD

SEQ ID NO: 348 - protein, 6 aa, synthetic construct
HDSFPY

SEQ ID NO: 349 - protein, 7 aa, synthetic construct
GFTLTKL

SEQ ID NO: 350 - protein, 4 aa, synthetic construct
PRDG

SEQ ID NO: 351 - protein, 11 aa, synthetic construct
GDYDYYYYYMG

SEQ ID NO: 352 - protein, 6 aa, synthetic construct
HDNFPY

SEQ ID NO: 353 - protein, 7 aa, synthetic construct
GHTVTEL

SEQ ID NO: 354 - protein, 4 aa, synthetic construct
PRDD

SEQ ID NO: 355 - protein, 11 aa, synthetic construct
GNPDYYFYYMD

SEQ ID NO: 356 - protein, 6 aa, synthetic construct
HDNFPL

SEQ ID NO: 357 - protein, 11 aa, synthetic construct
GNKDYYFYYMD

SEQ ID NO: 358 - protein, 7 aa, synthetic construct
GYTLTAL

SEQ ID NO: 359 - protein, 11 aa, synthetic construct
GDSDYYYYYMD

SEQ ID NO: 360 - protein, 4 aa, synthetic construct
PKYG

SEQ ID NO: 361 - protein, 11 aa, synthetic construct
GNYEYFYYYMD

SEQ ID NO: 362 - protein, 11 aa, synthetic construct
GSPDYFYYYMD

SEQ ID NO: 363 - protein, 4 aa, synthetic construct
PRNG

SEQ ID NO: 364 - protein, 11 aa, synthetic construct
GSPDYYYWYMD

SEQ ID NO: 365 - protein, 11 aa, synthetic construct
GSPDYYFWYMD

SEQ ID NO: 366 - protein, 7 aa, synthetic construct
GYTFTSY

SEQ ID NO: 367 - protein, 4 aa, synthetic construct
PNSG

SEQ ID NO: 368 - protein, 10 aa, synthetic construct
VLKGDYYYMD

SEQ ID NO: 369 - protein, 6 aa, synthetic construct
DDHLPL

SEQ ID NO: 370 - protein, 8 aa, synthetic construct
SQSISSTY

SEQ ID NO: 371 - protein, 8 aa, synthetic construct
GGSISGDY

SEQ ID NO: 372 - protein, 4 aa, synthetic construct
GSGG

SEQ ID NO: 373 - protein, 9 aa, synthetic construct
ILKYLHWLL

SEQ ID NO: 374 - protein, 8 aa, synthetic construct
GDNVGRKS

SEQ ID NO: 375 - protein, 8 aa, synthetic construct
WDSTADHW

SEQ ID NO: 376 - protein, 7 aa, synthetic construct
GYTFTDY

SEQ ID NO: 377 - protein, 4 aa, synthetic construct
PGSG

SEQ ID NO: 378 - protein, 7 aa, synthetic construct
GNGTGFA

SEQ ID NO: 379 - protein, 11 aa, synthetic construct
GQSVDYDGDSY

SEQ ID NO: 380 - protein, 6 aa, synthetic construct
SYKDPL

SEQ ID NO: 381 - protein, 4 aa, synthetic construct
PYND

SEQ ID NO: 382 - protein, 11 aa, synthetic construct
KDNYATGAWFA

SEQ ID NO: 383 - protein, 13 aa, synthetic construct
SQSLLYSTNQKNY

SEQ ID NO: 384 - protein, 5 aa, synthetic construct
YYSYR

SEQ ID NO: 385 - protein, 7 aa, synthetic construct
GFSFSDC

SEQ ID NO: 386 - protein, 6 aa, synthetic construct
VKSENY

SEQ ID NO: 387 - protein, 11 aa, synthetic construct
YYRYDVGAWFA

SEQ ID NO: 388 - protein, 11 aa, synthetic construct
SKSVSTSGYSY

SEQ ID NO: 389 - protein, 6 aa, synthetic construct
SRELPW

SEQ ID NO: 390 - protein, 7 aa, synthetic construct
GYTLTEL

SEQ ID NO: 391 - protein, 4 aa, synthetic construct
PEDG

SEQ ID NO: 392 - protein, 14 aa, synthetic construct
HNWNDGYYFYYYMD

SEQ ID NO: 393 - protein, 12 aa, synthetic construct
SQSLLHTDGKTY

SEQ ID NO: 394 - protein, 6 aa, synthetic construct
SLQLPL

SEQ ID NO: 395 - protein, 7 aa, synthetic construct
GFTFSDY

SEQ ID NO: 396 - protein, 4 aa, synthetic construct
SSGN

SEQ ID NO: 397 - protein, 14 aa, synthetic construct
GYNNYNYSYYYFMD

SEQ ID NO: 398 - protein, 7 aa, synthetic construct
SQSVSRK

SEQ ID NO: 399 - protein, 6 aa, synthetic construct
YNNWPY

SEQ ID NO: 400 - protein, 7 aa, synthetic construct
GFTVSSF

SEQ ID NO: 401 - protein, 4 aa, synthetic construct
YDGT

SEQ ID NO: 402 - protein, 11 aa, synthetic construct
IAVDGTDYYMD

SEQ ID NO: 403 - protein, 7 aa, synthetic construct
SQGIRSG

SEQ ID NO: 404 - protein, 6 aa, synthetic construct
DYNYPY

SEQ ID NO: 405 - protein, 4 aa, synthetic construct
SSGL

SEQ ID NO: 406 - protein, 14 aa, synthetic construct
GYSGFDDHYYYYTD

SEQ ID NO: 407 - protein, 7 aa, synthetic construct
SQSVSSN

SEQ ID NO: 408 - protein, 6 aa, synthetic construct
YKNWPY

SEQ ID NO: 409 - protein, 4 aa, synthetic construct
SSGS

SEQ ID NO: 410 - protein, 14 aa, synthetic construct
DYSNYEDYYYYYTG

SEQ ID NO: 411 - protein, 7 aa, synthetic construct
SQSVSSK

SEQ ID NO: 412 - protein, 4 aa, synthetic construct
SSGI

SEQ ID NO: 413 - protein, 14 aa, synthetic construct
DYSNYEDHYYYYTG

SEQ ID NO: 414 - protein, 7 aa, synthetic construct
SQSVSNK

SEQ ID NO: 415 - protein, 4 aa, synthetic construct
SSGK

SEQ ID NO: 416 - protein, 4 aa, synthetic construct
ISGQ

SEQ ID NO: 417 - protein, 14 aa, synthetic construct
GYSNYGVKYYYYMD

SEQ ID NO: 418 - protein, 7 aa, synthetic construct
SQSISSN

SEQ ID NO: 419 - protein, 6 aa, synthetic construct
YNAWTY

SEQ ID NO: 420 - protein, 7 aa, synthetic construct
GFTFSDH

SEQ ID NO: 421 - protein, 4 aa, synthetic construct
ISGN

SEQ ID NO: 422 - protein, 15 aa, synthetic construct
GYSSSSRGDYSYYTD

SEQ ID NO: 423 - protein, 4 aa, synthetic construct
ISGR

SEQ ID NO: 424 - protein, 7 aa, synthetic construct
SQGVSSN

SEQ ID NO: 425 - protein, 7 aa, synthetic construct
GFTVSSY

SEQ ID NO: 426 - protein, 4 aa, synthetic construct
FDGS

SEQ ID NO: 427 - protein, 11 aa, synthetic construct
VALEGYYYYMD

SEQ ID NO: 428 - protein, 7 aa, synthetic construct
SQDIRNG

SEQ ID NO: 429 - protein, 11 aa, synthetic construct
LALEGYYYYMD

SEQ ID NO: 430 - protein, 11 aa, synthetic construct
IALEGYYYYMD

SEQ ID NO: 431 - protein, 7 aa, synthetic construct
GFTFSSY

SEQ ID NO: 432 - protein, 4 aa, synthetic construct
YDGS

SEQ ID NO: 433 - protein, 4 aa, synthetic construct
YDGN

SEQ ID NO: 434 - protein, 7 aa, synthetic construct
SQGIRNG

SEQ ID NO: 435 - protein, 4 aa, synthetic construct
GSVG

SEQ ID NO: 436 - protein, 8 aa, synthetic construct
GYDWSGMD

SEQ ID NO: 437 - protein, 7 aa, synthetic construct
SQGIRND

SEQ ID NO: 438 - protein, 4 aa, synthetic construct
FDGT

SEQ ID NO: 439 - protein, 11 aa, synthetic construct
VAIQGRDYYID

SEQ ID NO: 440 - protein, 11 aa, synthetic construct
VAIQGYYYYMD

SEQ ID NO: 441 - protein, 7 aa, synthetic construct
GYTFTNY

SEQ ID NO: 442 - protein, 4 aa, synthetic construct
TNTG

SEQ ID NO: 443 - protein, 12 aa, synthetic construct
GIYYDYGYYAMD

SEQ ID NO: 444 - protein, 11 aa, synthetic construct
SESVDSYGNSF

SEQ ID NO: 445 - protein, 6 aa, synthetic construct
NNEDPY

SEQ ID NO: 446 - protein, 7 aa, synthetic construct
EIIFSTY

SEQ ID NO: 447 - protein, 4 aa, synthetic construct
GSGD

SEQ ID NO: 448 - protein, 8 aa, synthetic construct
GYNWNYMD

SEQ ID NO: 449 - protein, 6 aa, synthetic construct
DYTYPY

SEQ ID NO: 450 - protein, 7 aa, synthetic construct
GLTFSTF

SEQ ID NO: 451 - protein, 4 aa, synthetic construct
GSGE

SEQ ID NO: 452 - protein, 7 aa, synthetic construct
SHGIRND

SEQ ID NO: 453 - protein, 7 aa, synthetic construct
GFTFSTY

SEQ ID NO: 454 - protein, 4 aa, synthetic construct
DNIG

SEQ ID NO: 455 - protein, 7 aa, synthetic construct
NEDYYMD

SEQ ID NO: 456 - protein, 6 aa, synthetic construct
DYNYPR

SEQ ID NO: 457 - protein, 7 aa, synthetic construct
GFTFSTN

SEQ ID NO: 458 - protein, 4 aa, synthetic construct
GSNG

SEQ ID NO: 459 - protein, 8 aa, synthetic construct
RDNWNGFD

SEQ ID NO: 460 - protein, 7 aa, synthetic construct
SHVIRND

SEQ ID NO: 461 - protein, 11 aa, synthetic construct
GNEDYYFYYMD

SEQ ID NO: 462 - protein, 6 aa, synthetic construct
HDHFPY

SEQ ID NO: 463 - protein, 10 aa, synthetic construct
ASGFSFSNYA

SEQ ID NO: 464 - protein, 15 aa, synthetic construct
  VARIANT 3: note = A, D, E
  VARIANT 4: note = A, G, W, Y
  VARIANT 10: note = G, S
  VARIANT 15: note = A, P
INXXGSTFYXDSVKX

SEQ ID NO: 465 - protein, 10 aa, synthetic construct
HYGGSYDPMD

SEQ ID NO: 466 - protein, 8 aa, synthetic construct
ASQDINKY

SEQ ID NO: 467 - protein, 12 aa, synthetic construct
  VARIANT 4: note = I, T
YTSXLHPGVPSR

SEQ ID NO: 468 - protein, 15 aa, synthetic construct
  VARIANT 3: note = A, D, E
  VARIANT 4: note = A, G
  VARIANT 10: note = G, S
  VARIANT 15: note = A, P
INXXGSTFYXDSVKX

SEQ ID NO: 469 - protein, 12 aa, synthetic construct
YTSTLHPGVPSR

SEQ ID NO: 470 - protein, 14 aa, synthetic construct
  VARIANT 4: note = W, Y
INEXGSTFYADSVK

SEQ ID NO: 471 - protein, 16 aa, synthetic construct
INEGGSTFYADSVKSR

SEQ ID NO: 472 - protein, 16 aa, synthetic construct
INEWGSTFYADSVKSR

SEQ ID NO: 473 - protein, 16 aa, synthetic construct
INEYGSTFYADSVKSR

SEQ ID NO: 474 - protein, 12 aa, synthetic construct
YTSILHPGVPSR

SEQ ID NO: 475 - protein, 16 aa, synthetic construct
INEGGSTFYADSVKGR

SEQ ID NO: 476 - protein, 16 aa, synthetic construct
INAGGSTFYADSVKGR

SEQ ID NO: 477 - protein, 16 aa, synthetic construct
INDAGSTFYADSVKGR

SEQ ID NO: 478 - protein, 16 aa, synthetic construct
INDGGSTFYADSVKGR

SEQ ID NO: 479 - protein, 10 aa, synthetic construct
PSGFTFDDYG

SEQ ID NO: 480 - protein, 17 aa, synthetic construct
INWNGDSTGYADSVRGR

SEQ ID NO: 481 - protein, 8 aa, synthetic construct
DGAIGGMD

SEQ ID NO: 482 - protein, 8 aa, synthetic construct
ASQDIRDD

SEQ ID NO: 483 - protein, 12 aa, synthetic construct
SASTLQSGVPSR

SEQ ID NO: 484 - protein, 10 aa, synthetic construct
VSGHTLTELS

SEQ ID NO: 485 - protein, 17 aa, synthetic construct
FDPRGGETIYAQKFQGR

SEQ ID NO: 486 - protein, 12 aa, synthetic construct
GGDRDYYYYYMD

SEQ ID NO: 487 - protein, 8 aa, synthetic construct
ASQDIDDD

SEQ ID NO: 488 - protein, 12 aa, synthetic construct
EATTLVPGVPPR

SEQ ID NO: 489 - protein, 10 aa, synthetic construct
VSGFTLTKLS

SEQ ID NO: 490 - protein, 17 aa, synthetic construct
FDPRDGERIYAQKFQGR

SEQ ID NO: 491 - protein, 12 aa, synthetic construct
GGDYDYYYYYMG

SEQ ID NO: 492 - protein, 12 aa, synthetic construct
EATTLVPGIPPR

SEQ ID NO: 493 - protein, 10 aa, synthetic construct
VSGHTVTELS

SEQ ID NO: 494 - protein, 17 aa, synthetic construct
FHPRDDEIIYAQKFQGR

SEQ ID NO: 495 - protein, 12 aa, synthetic construct
GGNPDYYFYYMD

SEQ ID NO: 496 - protein, 17 aa, synthetic construct
FHPRDGETIYAQKFQGR

SEQ ID NO: 497 - protein, 12 aa, synthetic construct
GGNKDYYFYYMD

SEQ ID NO: 498 - protein, 10 aa, synthetic construct
VSGYTLTALS

SEQ ID NO: 499 - protein, 17 aa, synthetic construct
FNPRDGETIYAQKFQGR

SEQ ID NO: 500 - protein, 12 aa, synthetic construct
GGDSDYYYYYMD

SEQ ID NO: 501 - protein, 17 aa, synthetic construct
FNPRDDETIYTQKFQGR

SEQ ID NO: 502 - protein, 17 aa, synthetic construct
FDPKYGETTYAQKFQDR

SEQ ID NO: 503 - protein, 12 aa, synthetic construct
GGNYEYFYYYMD

SEQ ID NO: 504 - protein, 17 aa, synthetic construct
FDPKYGETMYAQKFQGR

SEQ ID NO: 505 - protein, 12 aa, synthetic construct
GGSPDYFYYYMD

SEQ ID NO: 506 - protein, 12 aa, synthetic construct
EATTLVPGIPRR

SEQ ID NO: 507 - protein, 17 aa, synthetic construct
FDPRNGETIYAQKFQGR

SEQ ID NO: 508 - protein, 12 aa, synthetic construct
GGSPDYYYWYMD

SEQ ID NO: 509 - protein, 12 aa, synthetic construct
GGSPDYYFWYMD

SEQ ID NO: 510 - protein, 10 aa, synthetic construct
ASGYTFTSYD

SEQ ID NO: 511 - protein, 17 aa, synthetic construct
MNPNSGNTGSTQKFQGR

SEQ ID NO: 512 - protein, 11 aa, synthetic construct
GVLKGDYYYMD

SEQ ID NO: 513 - protein, 9 aa, synthetic construct
ASQSISSSY

SEQ ID NO: 514 - protein, 12 aa, synthetic construct
GSSTRATGIPAR

SEQ ID NO: 515 - protein, 17 aa, synthetic construct
VNPNSGNTGSTQKFQGR

SEQ ID NO: 516 - protein, 9 aa, synthetic construct
ASQSISSTY

SEQ ID NO: 517 - protein, 11 aa, synthetic construct
VSGGSISGDYY

SEQ ID NO: 518 - protein, 17 aa, synthetic construct
IYGSGGGTNYNPSLNNR

SEQ ID NO: 519 - protein, 10 aa, synthetic construct
NILKYLHWLL

SEQ ID NO: 520 - protein, 12 aa, synthetic construct
ADSERPSGIPAR

SEQ ID NO: 521 - protein, 10 aa, synthetic construct
ASGYTFTDYV

SEQ ID NO: 522 - protein, 17 aa, synthetic construct
IYPGSGSAYSNAKFKDR

SEQ ID NO: 523 - protein, 8 aa, synthetic construct
RGNGTGFA

SEQ ID NO: 524 - protein, 12 aa, synthetic construct
AGQSVDYDGDSY

SEQ ID NO: 525 - protein, 12 aa, synthetic construct
VASNLESGIPAR

SEQ ID NO: 526 - protein, 10 aa, synthetic construct
ASGYTFTSYV

SEQ ID NO: 527 - protein, 17 aa, synthetic construct
INPYNDGTDYDEKFKGK

SEQ ID NO: 528 - protein, 12 aa, synthetic construct
EKDNYATGAWFA

SEQ ID NO: 529 - protein, 14 aa, synthetic construct
SSQSLLYSTNQKNY

SEQ ID NO: 530 - protein, 12 aa, synthetic construct
WASTRESGVPDR

SEQ ID NO: 531 - protein, 10 aa, synthetic construct
ASGFSFSDCR

SEQ ID NO: 532 - protein, 19 aa, synthetic construct
ISVKSENYGANYAESVRGR

SEQ ID NO: 533 - protein, 12 aa, synthetic construct
SYYRYDVGAWFA

SEQ ID NO: 534 - protein, 12 aa, synthetic construct
ASKSVSTSGYSY

SEQ ID NO: 535 - protein, 12 aa, synthetic construct
LASILESGVPDR

SEQ ID NO: 536 - protein, 10 aa, synthetic construct
VSGYTLTELS

SEQ ID NO: 537 - protein, 17 aa, synthetic construct
FDPEDGKTIYAPKFQGR

SEQ ID NO: 538 - protein, 15 aa, synthetic construct
GHNWNDGYYFYYYMD

SEQ ID NO: 539 - protein, 13 aa, synthetic construct
SSQSLLHTDGKTY

SEQ ID NO: 540 - protein, 12 aa, synthetic construct
EVSNRFSGVPDR

SEQ ID NO: 541 - protein, 10 aa, synthetic construct
ASGFTFSDYY

SEQ ID NO: 542 - protein, 17 aa, synthetic construct
ISSSGNTIFYVDSVEGR

SEQ ID NO: 543 - protein, 15 aa, synthetic construct
EGYNNYNYSYYYFMD

SEQ ID NO: 544 - protein, 8 aa, synthetic construct
ASQSVSRK

SEQ ID NO: 545 - protein, 12 aa, synthetic construct
AASTRATGIPAR

SEQ ID NO: 546 - protein, 10 aa, synthetic construct
ASGFTVSSFG

SEQ ID NO: 547 - protein, 17 aa, synthetic construct
IWYDGTNKYYADSVKGR

SEQ ID NO: 548 - protein, 12 aa, synthetic construct
EIAVDGTDYYMD

SEQ ID NO: 549 - protein, 8 aa, synthetic construct
ASQGIRSG

SEQ ID NO: 550 - protein, 12 aa, synthetic construct
AASTLQSGVPSR

SEQ ID NO: 551 - protein, 17 aa, synthetic construct
ISSSGLTIFYVDSVKGR

SEQ ID NO: 552 - protein, 15 aa, synthetic construct
EGYSGFDDHYYYYTD

SEQ ID NO: 553 - protein, 8 aa, synthetic construct
ASQSVSSN

SEQ ID NO: 554 - protein, 12 aa, synthetic construct
GASIRATGIPAR

SEQ ID NO: 555 - protein, 17 aa, synthetic construct
ISSSGSTIFYTDSVKGR

SEQ ID NO: 556 - protein, 15 aa, synthetic construct
EDYSNYEDYYYYYTG

SEQ ID NO: 557 - protein, 8 aa, synthetic construct
ASQSVSSK

SEQ ID NO: 558 - protein, 12 aa, synthetic construct
NASTRATGIPAR

SEQ ID NO: 559 - protein, 17 aa, synthetic construct
ISSSGITIFYTDSVKGR

SEQ ID NO: 560 - protein, 15 aa, synthetic construct
EDYSNYEDHYYYYTG

SEQ ID NO: 561 - protein, 8 aa, synthetic construct
ASQSVSNK

SEQ ID NO: 562 - protein, 12 aa, synthetic construct
NASTRATSIPAR

SEQ ID NO: 563 - protein, 12 aa, synthetic construct
GASTRATSIPAR

SEQ ID NO: 564 - protein, 17 aa, synthetic construct
ISSSGKTIFYTDSVKGR

SEQ ID NO: 565 - protein, 12 aa, synthetic construct
GASTRATGIPAR

SEQ ID NO: 566 - protein, 17 aa, synthetic construct
ISISGQTIYYGDSVKGR

SEQ ID NO: 567 - protein, 15 aa, synthetic construct
EGYSNYGVKYYYYMD

SEQ ID NO: 568 - protein, 8 aa, synthetic construct
ASQSISSN

SEQ ID NO: 569 - protein, 12 aa, synthetic construct
NASTRATDIPAR

SEQ ID NO: 570 - protein, 10 aa, synthetic construct
ASGFTFSDHY

SEQ ID NO: 571 - protein, 17 aa, synthetic construct
ISISGNTIYYTDSVKGR

SEQ ID NO: 572 - protein, 16 aa, synthetic construct
EGYSSSSRGDYSYYTD

SEQ ID NO: 573 - protein, 17 aa, synthetic construct
ISISGRTIYYIDSVKGR

SEQ ID NO: 574 - protein, 8 aa, synthetic construct
ASQGVSSN

SEQ ID NO: 575 - protein, 12 aa, synthetic construct
GASTRATDIPAR

SEQ ID NO: 576 - protein, 10 aa, synthetic construct
ASGFTVSSYG

SEQ ID NO: 577 - protein, 17 aa, synthetic construct
IWFDGSNKYYADSLTGL

SEQ ID NO: 578 - protein, 12 aa, synthetic construct
EVALEGYYYYMD

SEQ ID NO: 579 - protein, 8 aa, synthetic construct
ASQDIRNG

SEQ ID NO: 580 - protein, 12 aa, synthetic construct
TTSSLQTGVPSR

SEQ ID NO: 581 - protein, 17 aa, synthetic construct
LWFDGSNKFYADSVKGR

SEQ ID NO: 582 - protein, 12 aa, synthetic construct
ELALEGYYYYMD

SEQ ID NO: 583 - protein, 12 aa, synthetic construct
TTSSLQSGVPSR

SEQ ID NO: 584 - protein, 17 aa, synthetic construct
LWFDGSNQFYADSVKGR

SEQ ID NO: 585 - protein, 12 aa, synthetic construct
EIALEGYYYYMD

SEQ ID NO: 586 - protein, 8 aa, synthetic construct
SSQDIRNG

SEQ ID NO: 587 - protein, 10 aa, synthetic construct
ASGFTFSSYG

SEQ ID NO: 588 - protein, 17 aa, synthetic construct
IWYDGSNKYYADSVKGR

SEQ ID NO: 589 - protein, 12 aa, synthetic construct
TTSNLQSGVPSR

SEQ ID NO: 590 - protein, 17 aa, synthetic construct
IWYDGNNKYYADSVKGR

SEQ ID NO: 591 - protein, 8 aa, synthetic construct
ASQGIRNG

SEQ ID NO: 592 - protein, 12 aa, synthetic construct
AASSLQSGVPSS

SEQ ID NO: 593 - protein, 10 aa, synthetic construct
ASGFTFSSYA

SEQ ID NO: 594 - protein, 17 aa, synthetic construct
ISGSVGSTYYADSVKGR

SEQ ID NO: 595 - protein, 9 aa, synthetic construct
EGYDWSGMD

SEQ ID NO: 596 - protein, 8 aa, synthetic construct
ASQGIRND

SEQ ID NO: 597 - protein, 12 aa, synthetic construct
AASSLQSGVPSR

SEQ ID NO: 598 - protein, 17 aa, synthetic construct
IWFDGTTRFYADSVKGR

SEQ ID NO: 599 - protein, 12 aa, synthetic construct
EVAIQGRDYYID

SEQ ID NO: 600 - protein, 17 aa, synthetic construct
IWFDGTNKFYADSVKGR

SEQ ID NO: 601 - protein, 12 aa, synthetic construct
EVAIQGYYYYMD

SEQ ID NO: 602 - protein, 12 aa, synthetic construct
TTSNLQNGVPSR

SEQ ID NO: 603 - protein, 10 aa, synthetic construct
ASGYTFTNYG

SEQ ID NO: 604 - protein, 17 aa, synthetic construct
INTNTGEPTYAEEFKGR

SEQ ID NO: 605 - protein, 13 aa, synthetic construct
LGIYYDYGYYAMD

SEQ ID NO: 606 - protein, 12 aa, synthetic construct
ASESVDSYGNSF

SEQ ID NO: 607 - protein, 12 aa, synthetic construct
LASNLESGVPAR

SEQ ID NO: 608 - protein, 10 aa, synthetic construct
ASEIIFSTYA

SEQ ID NO: 609 - protein, 17 aa, synthetic construct
ISGSGDNTYYADSVKGR

SEQ ID NO: 610 - protein, 9 aa, synthetic construct
EGYNWNYMD

SEQ ID NO: 611 - protein, 12 aa, synthetic construct
EASSLQSGVPSR

SEQ ID NO: 612 - protein, 10 aa, synthetic construct
ASGLTFSTFA

SEQ ID NO: 613 - protein, 17 aa, synthetic construct
ISGSGENTYYADSVKGR

SEQ ID NO: 614 - protein, 8 aa, synthetic construct
ASHGIRND

SEQ ID NO: 615 - protein, 12 aa, synthetic construct
ETSSLQSGVPSR

SEQ ID NO: 616 - protein, 10 aa, synthetic construct
ASGFTFSTYA

SEQ ID NO: 617 - protein, 17 aa, synthetic construct
ISDNIGNTYYADSVKGR

SEQ ID NO: 618 - protein, 8 aa, synthetic construct
DNEDYYMD

SEQ ID NO: 619 - protein, 10 aa, synthetic construct
ASGFTFSTNA

SEQ ID NO: 620 - protein, 17 aa, synthetic construct
ISGSNGNTYYADSVKGR

SEQ ID NO: 621 - protein, 9 aa, synthetic construct
ERDNWNGFD

SEQ ID NO: 622 - protein, 8 aa, synthetic construct
ASHVIRND

SEQ ID NO: 623 - protein, 17 aa, synthetic construct
FDPRDGQIIYAEKFQGR

SEQ ID NO: 624 - protein, 12 aa, synthetic construct
GGNEDYYFYYMD

SEQ ID NO: 625 - protein, 119 aa, synthetic construct
  VARIANT 53: note = A, D, E
  VARIANT 54: note = A, G, W, Y
  VARIANT 60: note = A, P
  VARIANT 65: note = G, S
EVQLLESGGGLVQPGGSLRLSCAASGFSFSNYAMSWVRQAPGKGLEWVASINXXGSTFYXDSVKXRFTISRDNSKNTLYLEMNSLRAEDTAVFYCSRHYGGSYDPMDYWGQGTTVTVSS

SEQ ID NO: 626 - protein, 107 aa, synthetic construct
  VARIANT 24: note = K, R
  VARIANT 53: note = I, T
  VARIANT 96: note = F, Q, Y
DIQMTQSPSSLSASVGDRVTITCXASQDINKYIAWYQQKPGKGPKLLIHYTSXLHPGVPSRFSGSGSGTDYTLTISSLQPEDFATYYCLQYDNPLXTFGQGTKLEIK

SEQ ID NO: 627 - protein, 119 aa, synthetic construct; identical to SEQ ID NO: 625
  VARIANT 53: note = A, D, E
  VARIANT 54: note = A, G
  VARIANT 60: note = A, P
  VARIANT 65: note = G, S

SEQ ID NO: 628 - protein, 107 aa, synthetic construct
  VARIANT 24: note = K, R
DIQMTQSPSSLSASVGDRVTITCXASQDINKYIAWYQQKPGKGPKLLIHYTSTLHPGVPSRFSGSGSGTDYTLTISSLQPEDFATYYCLQYDNPLYTFGQGTKLEIK

SEQ ID NO: 629 - protein, 119 aa, synthetic construct
  VARIANT 54: note = W, Y
EVQLLESGGGLVQPGGSLRLSCAASGFSFSNYAMSWVRQAPGKGLEWVASINEXGSTFYADSVKSRFTISRDNSKNTLYLEMNSLRAEDTAVFYCSRHYGGSYDPMDYWGQGTTVTVSS

SEQ ID NO: 630 - protein, 107 aa, synthetic construct
  VARIANT 53: note = I, T
  VARIANT 96: note = F, Q, Y
DIQMTQSPSSLSASVGDRVTITCRASQDINKYIAWYQQKPGKGPKLLIHYTSXLHPGVPSRFSGSGSGTDYTLTISSLQPEDFATYYCLQYDNPLXTFGQGTKLEIK

SEQ ID NO: 631 - protein, 107 aa, synthetic construct
  VARIANT 96: note = F, Q, Y
DIQMTQSPSSLSASVGDRVTITCRASQDINKYIAWYQQKPGKGPKLLIHYTSILHPGVPSRFSGSGSGTDYTLTISSLQPEDFATYYCLQYDNPLXTFGQGTKLEIK

SEQ ID NO: 632 - protein, 107 aa, synthetic construct
DIQMTQSPSSLSASVGDRVTITCRASQDINKYIAWYQQKPGKGPKLLIHYTSTLHPGVPSRFSGSGSGTDYTLTISSLQPEDFATYYCLQYDNPLYTFGQGTKLEIK

SEQ ID NO: 633 - protein, 119 aa, synthetic construct
  VARIANT 65: note = G, S
EVQLLESGGGLVQPGGSLRLSCAASGFSFSNYAMSWVRQAPGKGLEWVASINEGGSTFYADSVKXRFTISRDNSKNTLYLEMNSLRAEDTAVFYCSRHYGGSYDPMDYWGQGTTVTVSS

SEQ ID NO: 634 - protein, 119 aa, synthetic construct
EVQLLESGGGLVQPGGSLRLSCAASGFSFSNYAMSWVRQAPGKGLEWVASINEGGSTFYADSVKSRFTISRDNSKNTLYLEMNSLRAEDTAVFYCSRHYGGSYDPMDYWGQGTTVTVSS

SEQ ID NO: 635 - protein, 119 aa, synthetic construct
EVQLLESGGGLVQPGGSLRLSCAASGFSFSNYAMSWVRQAPGKGLEWVASINEWGSTFYADSVKSRFTISRDNSKNTLYLEMNSLRAEDTAVFYCSRHYGGSYDPMDYWGQGTTVTVSS

SEQ ID NO: 636 - protein, 119 aa, synthetic construct
EVQLLESGGGLVQPGGSLRLSCAASGFSFSNYAMSWVRQAPGKGLEWVASINEYGSTFYADSVKSRFTISRDNSKNTLYLEMNSLRAEDTAVFYCSRHYGGSYDPMDYWGQGTTVTVSS

SEQ ID NO: 637 - protein, 107 aa, synthetic construct
DIQMTQSPSSLSASVGDRVTITCRASQDINKYIAWYQQKPGKGPKLLIHYTSILHPGVPSRFSGSGSGTDYTLTISSLQPEDFATYYCLQYDNPLQTFGQGTKLEIK

SEQ ID NO: 638 - protein, 107 aa, synthetic construct
DIQMTQSPSSLSASVGDRVTITCRASQDINKYIAWYQQKPGKGPKLLIHYTSILHPGVPSRFSGSGSGTDYTLTISSLQPEDFATYYCLQYDNPLFTFGQGTKLEIK

SEQ ID NO: 639 - protein, 119 aa, synthetic construct
EVQLLESGGGLVQPGGSLRLSCAASGFSFSNYAMSWVRQAPGKGLEWVASINEGGSTFYADSVKGRFTISRDNSKNTLYLQMNSLRAEDTAVFYCSRHYGGSYDPMDYWGQGTTVTVSS

SEQ ID NO: 640 - protein, 119 aa, synthetic construct
EVQLLESGGGLVQPGGSLRLSCAASGFSFSNYAMSWVRQAPGKGLEWVASINDGGSTFYADSVKGRFTISRDNSKNTLYLQMNSLRAEDTAVFYCSRHYGGSYDPMDYWGQGTTVTVSS

SEQ ID NO: 641 - protein, 119 aa, synthetic construct
EVQLLESGGGLVQPGGSLRLSCAASGFSFSNYAMSWVRQAPGKGLEWVASINDGGSTFYADSVKGRFTISRDNSKNTLYLQMNSLRAEDTAVYYCSRHYGGSYDPMDYWGQGTTVTVSS

SEQ ID NO: 642 - protein, 119 aa, synthetic construct
EVQLLESGGGLVQPGGSLRLSCAASGFSFSNYAMSWVRQAPGKGLEWVASINAGGSTFYADSVKGRFTISRDNSKNTLYLQMNSLRAEDTAVFYCSRHYGGSYDPMDYWGQGTTVTVSS

SEQ ID NO: 643 - protein, 119 aa, synthetic construct
EVQLLESGGGLVQPGGSLRLSCAASGFSFSNYAMSWVRQAPGKGLEWVASINDAGSTFYADSVKGRFTISRDNSKNTLYLQMNSLRAEDTAVFYCSRHYGGSYDPMDYWGQGTTVTVSS

SEQ ID NO: 644 - protein, 107 aa, synthetic construct
DIQMTQSPSSLSASVGDRVTITCRASQDINKYIAWYQQKPGKAPKLLIHYTSTLHPGVPSRFSGSGSGTDYTLTISSLQPEDFATYYCLQYDNPLYTFGQGTKLEIK

SEQ ID NO: 645 - protein, 107 aa, synthetic construct
DIQMTQSPSSLSASVGDRVTITCRASQDINKYIAWYQHKPGKAPKLLIHYTSTLHPGVPSRFSGSGSGTDYTLTISSLQPEDFATYYCLQYDNPLYTFGQGTKLEIK

SEQ ID NO: 646 - protein, 107 aa, synthetic construct
DIQMTQSPSSLSASVGDRVTITCRASQDINKYIAWYQHKPGKGPKLLIHYTSTLHPGVPSRFSGSGSGTDYTLTISSLQPEDFATYYCLQYDNPLYTFGQGTKLEIK

SEQ ID NO: 647 - protein, 118 aa, synthetic construct
EVQLVESGGGVVRPGGSLRLSCAPSGFTFDDYGMSWVRQAPGKGLEWVSGINWNGDSTGYADSVRGRFTISRDNAKNSLYLQMNSLRAEDTALYHCARDGAIGGMDVWGKGTTVTVSS

SEQ ID NO: 648 - protein, 107 aa, synthetic construct
AIQMTQSPSSLSASVGDRVTITCRASQDIRDDLGWYQQKPGKAPKLLIYSASTLQSGVPSRFSGSESGTDFTLTISSLQPEDFAAYYCLQDYNYPWTFGQGTRVEIK

SEQ ID NO: 649 - protein, 122 aa, synthetic construct
QVQLVQSGAEVRKPGASVKVSCKVSGHTLTELSMHWIRQAPGEGLEWMGYFDPRGGETIYAQKFQGRVTLTEDTSTDTAYMELTSLTSEDTAVYYCATGGDRDYYYYYMDVWGKGTTVTVSS

SEQ ID NO: 650 - protein, 107 aa, synthetic construct
ETTLTQSPAFMSATPGDKVNISCKASQDIDDDMNWFQRKPGEAAIFIIQEATTLVPGVPPRFSGSGYGTDFTLTISNIESEDAAYYFCLQHDSFPYTFGQGTKLEIK

SEQ ID NO: 651 - protein, 122 aa, synthetic construct
QVQLLQSGAEVKRPGASVRVSCKVSGFTLTKLSMHWVRQAPGKGLEWMGFFDPRDGERIYAQKFQGRVTMTEDTSTDTAYMELSSLRSEDTAVYYCATGGDYDYYYYYMGVWGKGTTVTVSS

SEQ ID NO: 652 - protein, 107 aa, synthetic construct
ETTLTQSPAFMSATPGDKVNISCKASQDIDDDLNWYQQKPGEAAIFIVQEATTLVPGIPPRFSGSGYGTDFTLTINNIESEDAAYYFCLQHDNFPYTFGQGTKLEIK

SEQ ID NO: 653 - protein, 122 aa, synthetic construct
QVQLVQSGAEVKKPGASVKVSCKVSGHTVTELSMHWVRQAPGKGLEWMGSFHPRDDEIIYAQKFQGRVTMTEDSFTDTAYMELSSLTSEDTAVYYCATGGNPDYYFYYMDVWGKGTPVTVSS

SEQ ID NO: 654 - protein, 107 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTFGQGTKVEIK

SEQ ID NO: 655 - protein, 122 aa, synthetic construct
QVQLLQSGAEVKKPGASVKVSCKVSGHTLTELSMHWVRQAPGKGLEWMGSFHPRDGETIYAQKFQGRVTLTEDTSKDTAFMELSRLRSEDTAVYYCATGGNKDYYFYYMDVWGKGTTVTVSS

SEQ ID NO: 656 - protein, 107 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTFGGGTKVEIK

SEQ ID NO: 657 - protein, 122 aa, synthetic construct
QVQLVQSGAEVKKPGASVKVSCKVSGYTLTALSMHWVRQAPGKGLEWMGYFNPRDGETIYAQKFQGRVTMTEDTFTDTAYMELSSLRSEDTAIYYCATGGDSDYYYYYMDVWGKGTTVTVSS

SEQ ID NO: 658 - protein, 107 aa, synthetic construct
ETTLTQSPAFMSATPGDKVNISCKASQDIDDDMNWYQQKPGEAAIFIIQEATTLVPGIPPRFSGSGYGTDFTLIINNIESEDAAYYFCLQHDNFPYTFGQGTKLEIK

SEQ ID NO: 659 - protein, 122 aa, synthetic construct
QVQLVQSGAEVKKPGASVKVSCKVSGHTLTELSMHWVRQAPGTGLEWMGYFNPRDDETIYTQKFQGRVTMTEDTSTDTAYMELRSLRSEDTAVYYCATGGNKDYYFYYMDVWGKGTTVTVSS

SEQ ID NO: 660 - protein, 107 aa, synthetic construct
ETTLTQSPAFMSATPGDKVTISCKASQDIDDDMNWYQQKPGEAAIFIIQEATTLVPGIPPRFSGSGYGTDFTLTINNIESEDAAYYFCLQHDNFPLTFGQGTKVEIK

SEQ ID NO: 661 - protein, 122 aa, synthetic construct
QVQLVQSGAEVKKPGASVKVSCKVSGHTLTELSMHWVRQAPGKGLEWMGVFDPKYGETTYAQKFQDRVTMTEDTSTDTAYMELSSLRSEDTAVYYCATGGNYEYFYYYMDVWGKGTTVTVSS

SEQ ID NO: 662 - protein, 107 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPYTFGQGTKLEIK

SEQ ID NO: 663 - protein, 122 aa, synthetic construct
QVQLIQSGAEVKKPGASVKVSCKVSGHTLTELSMHWVRQAPGKGLEWMGIFDPKYGETMYAQKFQGRVTLTEDTSTDTAYMELSSLRSEDTAVYYCATGGSPDYFYYYMDVWGKGTTVTVSS

SEQ ID NO: 664 - protein, 107 aa, synthetic construct
ETTLTQSPAFMSATPGDKVNISCKASQDIDDDMNWYQQKPGEAAIFVIQEATTLVPGIPRRFSGSGYGTDFTLTINNIESEDAAYYFCLQHDNFPYTFGQGTKLEIK

SEQ ID NO: 665 - protein, 122 aa, synthetic construct
QVQLLQSGAEVKKPGASVKVSCKVSGHTLTELSMHWVRQAPGKGLEWMGIFDPRNGETIYAQKFQGRVTMTEDTSTDTAYMELSSLRSEDTAVYYCATGGSPDYYYWYMDVWGKGTTVTVSS

SEQ ID NO: 666 - protein, 107 aa, synthetic construct
ETTLTQSPAFMSATPGDKVNISCKASQDIDDDMNWYQQKPGEPAIFIIQEATTLVPGIPPRFSGSGYGTDFTLTINNIESEDAAYYFCLQHDNFPYTFGQGTKLEIK

SEQ ID NO: 667 - protein, 122 aa, synthetic construct
QVQLLQSGAEVKKPGASVKVSCKVSGHTLTELSMHWVRQAPGKGLEWMGIFDPRNGETIYAQKFQGRVTMTEDTSTDTAYMELSSLSSEDTAVYYCATGGSPDYYFWYMDVWGKGTTVTVSS

SEQ ID NO: 668 - protein, 121 aa, synthetic construct
QVQLVQSGAEVKKPGASVKVSCKASGYTFTSYDINWVRQATGQGLEWMGWMNPNSGNTGSTQKFQGRVTMTRNTSITTAYMELSSLRSEDTAVYYCARGVLKGDYYYMDVWGKGTTVTVSS

SEQ ID NO: 669 - protein, 108 aa, synthetic construct
EIVMTQSPATLSLSPGERATLSCRASQSISSSYLSWYQQKPGQAPRLLIYGSSTRATGIPARFSGSGSGTDFTLTISSLQPEDFAVYYCQQDDHLPLTFGGGTKVEIK

SEQ ID NO: 670 - protein, 121 aa, synthetic construct
QVQLVHSGAEVKKPGASVKVSCKASGYTFTSYDINWVRQATGQGLEWMGWVNPNSGNTGSTQKFQGRVTMTRNTSISTAYMELSSLRSEDTAVYYCARGVLKGDYYYMDVWGKGTTVTVSS

SEQ ID NO: 671 - protein, 108 aa, synthetic construct
EIVMTQSPATLSLSPGERATLSCRASQSISSTYLSWYQQKPGQAPRLLIYGSSTRATGIPARFSGSGSGTDFTLTISSLQPEDFAVYYCQQDDHLPLTFGGGTKVEIK

SEQ ID NO: 672 - protein, 121 aa, synthetic construct
QVQLVQSGAEVKKPGASVKVSCKASGYTFTSYDINWVRQATGQGLEWMGWVNPNSGNTGSTQKFQGRVTMTRNTSISTAYMELSSLRSEDTAVYYCARGVLKGDYYYMDVWGKGTTVTVSS

SEQ ID NO: 673 - protein, 108 aa, synthetic construct
EIVMTQSPATLSLSPGERATLSCRASQSISSTYLSWYQQKPGQAPRLLIYGSSTRATGIPARFRGSGSGTDFTLTISSLQPEDFAVYYCQQDDHLPLTFGGGTKVEIK

SEQ ID NO: 674 - protein, 139 aa, synthetic construct
MKHLWFFLLLVAAPRWVLSQVQLQEAGPGLVKPSETLSLTCSVSGGSISGDYYWFWIRQSPGKGLEWIGYIYGSGGGTNYNPSLNNRVSISIDTSKNLFSLKLRSVTAADTAVYYCASNILKYLHWLLYWGQGVLVTVS

SEQ ID NO: 675 - protein, 128 aa, synthetic construct
MAWALLLLGLLAHFTDSAASYELSQPRSVSVSPGQTAGFTCGGDNVGRKSVQWYQQKPPQAPVLVIYADSERPSGIPARFSGSNSGNTATLTISGVEAGDEADYYCQVWDSTADHWVFGGGTRLTVLG

SEQ ID NO: 676 - protein, 118 aa, synthetic construct
QVQLVQSGPELKKPGASVKVSCKASGYTFTDYVIHWVKQATGQGLEWIGEIYPGSGSAYSNAKFKDRVTMTADKSSNTAYMELSSLTSDDTAVYFCARRGNGTGFAYWGQGTLVTVSS

SEQ ID NO: 677 - protein, 111 aa, synthetic construct
DIVLTQSPASLAVSLGQRATITCKAGQSVDYDGDSYMNWYQQKPGQPPKLLIYVASNLESGIPARFSGSGSGTDFTLNIHPVEENDAATYYCQQSYKDPLTFGQGTKLEIK

SEQ ID NO: 678 - protein, 122 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSS

SEQ ID NO: 679 - protein, 112 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIK

SEQ ID NO: 680 - protein, 124 aa, synthetic construct
EEQLVESGGGLVKPGGSLRLSCAASGFSFSDCRMYWLRQAPGKGLEWIGVISVKSENYGANYAESVRGRFTISRDDSKNTVYLQMNSLKTEDTAVYYCSASYYRYDVGAWFAYWGQGTLVTVSS

SEQ ID NO: 681 - protein, 111 aa, synthetic construct
DIVMTQSPDSLAVSLGERATINCRASKSVSTSGYSYIYWYQQKPGQPPKLLIYLASILESGVPDRFSGSGSGTDFTLTISSLQAEDVAVYYCQHSRELPWTFGQGTKVEIK

SEQ ID NO: 682 - protein, 125 aa, synthetic construct
QVQLVQSGAEVKKPGASVKVSCKVSGYTLTELSMHWVRQAPEKGLEWMGGFDPEDGKTIYAPKFQGRVTMTEDTSTDTAYMDLSSLRSEDTAVYYCATGHNWNDGYYFYYYMDLWGKGTTVTVSS

SEQ ID NO: 683 - protein, 112 aa, synthetic construct
DIVMTQTPLSLSVTPGQPASISCKSSQSLLHTDGKTYLYWYLQKPGQPPHLLIYEVSNRFSGVPDRLSGSGSGTDFTLKISRVEAEDVGVYYCMQSLQLPLTFGGGTMVEIK

SEQ ID NO: 684 - protein, 125 aa, synthetic construct
QVQLVESGGDSVKPGGSLRLSCAASGFTFSDYYMNWIRQAPGKGLEWISYISSSGNTIFYVDSVEGRFTVSRDNAKNSLYLQMNSLRAEDTAVYYCAREGYNNYNYSYYYFMDVWGKGTTVTVSS

SEQ ID NO: 685 - protein, 107 aa, synthetic construct
EIVMTQSPATLSVSPGERATLSCRASQSVSRKLAWYQQKPGQAPRLLIYAASTRATGIPARFSGSGSGTEFTLTISSLQSEDFAVYFCQQYNNWPYTFGRGTKLEIK

SEQ ID NO: 686 - protein, 122 aa, synthetic construct
QVQLVESGGGVVQPGRSLRLSCAASGFTVSSFGMHWVRQAPGKGLEWVAIIWYDGTNKYYADSVKGRFTISRDNSKNTLYLQLNSLRGEDTAVYYCAREIAVDGTDYYMDVWGRGTTVTVSS

SEQ ID NO: 687 - protein, 107 aa, synthetic construct
AIQMTQSPSSLSASVGDRVTFTCRASQGIRSGLGWYQQKPGKAPNLLIYAASTLQSGVPSRFSGSGSGTDFTLTITSLQPEDFATYYCLQDYNYPYTFGQGTKLEIK

SEQ ID NO: 688 - protein, 125 aa, synthetic construct
QVQLVESGGGLVTPGGSLRLSCAASGFTFSDYYMNWIRQAPGKGLEWLSYISSSGLTIFYVDSVKGRFTVSRDNAKNSLYLQMNSLRAEDTAVYYCAREGYSGFDDHYYYYTDVWGKGTTVTVSS

SEQ ID NO: 689 - protein, 107 aa, synthetic construct
EIVMTQSPATLSVSPGERATLSCRASQSVSSNLAWYQQKPGQAPRLLIYGASIRATGIPARFSGSGSGTEFTLTISSLQSEDFAVYYCQHYKNWPYTFGQGTRLEIK

SEQ ID NO: 690 - protein, 125 aa, synthetic construct
QVQLVESGGGLVKPGGSLRLSCAASGFTFSDYYMNWIRQAPGKGLEWVSYISSSGSTIFYTDSVKGRFTISRDNAKNSLYLQMNSLRAEDTAVYYCAREDYSNYEDYYYYYTGVWGKGTTVTVSS

SEQ ID NO: 691 - protein, 107 aa, synthetic construct
EIVMTQSPAALSVSPGERATLSCRASQSVSSKFAWYQQKPGQAPRLLIYNASTRATGIPARFSGSGSGTEFTLTISSLQSEDFAVYYCQQYNNWPYTFGQGTKLEIK

SEQ ID NO: 692 - protein, 125 aa, synthetic construct
QVQLVESGGGLVKPGGSLRLSCSASGFTFSDYYMNWIRQAPGKGLEWVSYISSSGITIFYTDSVKGRFTISRDNAKNSLYLQMNSLRAEDTAVYYCAREDYSNYEDHYYYYTGVWGKGTTVTVSS

SEQ ID NO: 693 - protein, 107 aa, synthetic construct
EIVMTQSPAALSVSPGERATLSCRASQSVSNKFAWYQQKPGQAPRLLIYNASTRATSIPARFSGSGSGTEFTLTISSLQSEDFAVYYCQHYNNWPYTFGQGTKLEIK

SEQ ID NO: 694 - protein, 107 aa, synthetic construct
EIVMTQSPAALSVSPGERATLSCRASQSVSNKFAWYQQKPGQAPRLLIYGASTRATSIPARFSGSGSGTEFTLTISSLQSEDFAVYYCQHYNNWPYTFGQGTKLEIK

SEQ ID NO: 695 - protein, 125 aa, synthetic construct
QVQLVESGGGLVKPGGSLRLSCAASGFTFSDYYMNWIRQAPGKGLEWVSYISSSGKTIFYTDSVKGRFTISRDNAKNSLFLQMNSLRAEDTAVYYCAREDYSNYEDYYYYYTGVWGKGTTVTVSS

SEQ ID NO: 696 - protein, 107 aa, synthetic construct
EIVMTQSPAALSVSPGERATLSCRASQSVSSKFAWYQQKPGQAPRLLIYNASTRATGIPARFSGSGSGTEFTLTISSLQSEDFAVYYCHQYKNWPYTFGQGTKLEIK

SEQ ID NO: 697 - protein, 107 aa, synthetic construct
EIVMTQSPAALSVSPGERATLSCRASQSVSSKFAWYQQKPGQAPRLLIYGASTRATGIPARFSGSGSGTEFTLTISSLQSEDFAVYYCHQYKNWPYTFGQGTKLEIK

SEQ ID NO: 698 - protein, 125 aa, synthetic construct
QVQLVESGGGLVKPGGSLRLSCAASGFTFSDYYMNWIRQAPGKGLEWISYISISGQTIYYGDSVKGRFTVSRDNAKNSLFLEMNSLRAEDSAVYFCAREGYSNYGVKYYYYMDVWGKGTTVTVSS

SEQ ID NO: 699 - protein, 107 aa, synthetic construct
EILMTQSPATLSVSPGERTTLSCRASQSISSNLAWYQQNPGQAPRLLIYNASTRATDIPARFSGGGSGTEFTLTISSLQSEDFAVYYCQQYNAWTYTFGQGTKLEIK

SEQ ID NO: 700 - protein, 126 aa, synthetic construct
QVQLVESGGGLVKPGGSLRLSCAASGFTFSDHYMNWVRQVPGKGLEWISYISISGNTIYYTDSVKGRFTVSRDNAKNSLYLQMTSLRAEDTAVYYCVREGYSSSSRGDYSYYTDVWGKGTTVTVSS

SEQ ID NO: 701 - protein, 107 aa, synthetic construct
EIMMTQSPATLSVSPGERATLSCRASQSVSSNLAWYQQKPGQAPRLLIYGASTRATGIPARFSGSGSGTEFTLTISSLQSEDFAVYYCQQYKNWPYTFGQGTKLEIK

SEQ ID NO: 702 - protein, 126 aa, synthetic construct
QVQLVESGGGLVKPGGSLRLSCAASGFTFSDYYMNWVRQVPGKGLEWISYISISGRTIYYIDSVKGRFTVSRDNAKNSLYLQMTNLRAEDTAVYYCVREGYSSSSRGDYSYYTDVWGKGTTVTVSS

SEQ ID NO: 703 - protein, 107 aa, synthetic construct
EIVMTQSPATLSASPGERVNLSCRASQGVSSNLAWYQQKVGQAPRLLIYGASTRATDIPARFSGSGSGSEFTLTISSLQSEDFAVYYCQHYKNWPYTFGQGTKLEIK

SEQ ID NO: 704 - protein, 122 aa, synthetic construct
QVQLVESGGGVVQPGRSLRLSCAASGFTVSSYGMHWVRQAPGKGLEWVAIIWFDGSNKYYADSLTGLFTISRDSSKNTLFLQMNSLSLEDTAVYYCAREVALEGYYYYMDVWGKGTTVTVSS

SEQ ID NO: 705 - protein, 107 aa, synthetic construct
AIQMTQSPSSLSASVGDRVTITCRASQDIRNGLGWYQQKPGKAPKLLIYTTSSLQTGVPSRFSGSGSGTDFTLTISSLQPEDFATYYCLQDYNYPYTFGQGTKLEIK

SEQ ID NO: 706 - protein, 122 aa, synthetic construct
QVQLVESGGGVVQPGRSLSLSCAASGFTVSSYGMHWVRQAPGKGLEWVAILWFDGSNKFYADSVKGRFTISRDNSKNTLYLQMNSLRVEDTAVYYCARELALEGYYYYMDVWGKGTTVTVSS

SEQ ID NO: 707 - protein, 107 aa, synthetic construct
AIQMTQSPSSLSASVGDRVTITCRASQDIRNGLGWYQQNPGKAPKLLIYTTSSLQSGVPSRFSGSGSGTDFTLTISSLQPEDFATYYCLQDYNYPYTFGQGTKLEIK

SEQ ID NO: 708 - protein, 122 aa, synthetic construct
QVQLVESGGGVVQPGRSLRLSCAASGFTVSSYGMHWVRQAPGKGLEWVAILWFDGSNQFYADSVKGRFTISRDNSKNTLYLQMNSLRVEDTAVYYCAREIALEGYYYYMDVWGKGTTVTVSS

SEQ ID NO: 709 - protein, 107 aa, synthetic construct
AIQMTQSPSSLSASVGDRVTITCRSSQDIRNGLGWYQQKPGKAPKLLIYTTSSLQSGVPSRFSGSGSGTDFTLTISSLQPEDFATYYCLQDYNYPYTFGQGTKLEIK

SEQ ID NO: 710 - protein, 122 aa, synthetic construct
QVQLVESGGGVVQPGRALRLSCAASGFTFSSYGMHWVRQAPGKGLEWVAIIWYDGSNKYYADSVKGRFTISRDNSKNTLYLQMNSLRAEDTAVYYCAREVALEGYYYYMDVWGKGTTVTVSS

SEQ ID NO: 711 - protein, 107 aa, synthetic construct
DIQMTQSPSSLSASVGDRVTITCRASQDIRNGLGWYQQKPGKAPKLLIYTTSNLQSGVPSRFSGSGSGTDFTLTISSLQPEDFATYYCLQDYNYPYTFGQGTKLEIK

SEQ ID NO: 712 - protein, 122 aa, synthetic construct
QVQLVESGGGVVQPGRSLRLSCAASGFTFSSYGMHWVRQAPGKGLEWVAIIWYDGNNKYYADSVKGRFTISRDNSKNTLYLQMNSLRAEDTAVYYCTREVALEGYYYYMDVWGKGTTVTVSS

SEQ ID NO: 713 - protein, 107 aa, synthetic construct
AIQMTQSPSSLSASVGDRVTITCRASQGIRNGLGWYQLKPGKAPKLLIYAASSLQSGVPSSFSGSGSGTDFTLTISSLQPEDFATYYCLQDYNYPYTFGQGTKLEIK

SEQ ID NO: 714 - protein, 119 aa, synthetic construct
EVQLLESGGGLVQPGGSLRLSCAASGFTFSSYAMSWVRQAPGKGLEWVSGISGSVGSTYYADSVKGRFTISRDKSKNTLYLQMNSLRAEDTAVYYCAKEGYDWSGMDVWGKGTTVTVSS

SEQ ID NO: 715 - protein, 107 aa, synthetic construct
AIQMTQSPSSLSAFVGDRVTITCRASQGIRNDLGWYQQKPGKAPKLLIYAASSLQSGVPSRFSGSGSGTDFTLTISSLQPEDFATYYCLQDYNYPYTFGQGTKLEIK

SEQ ID NO: 716 - protein, 122 aa, synthetic construct
QVLLVESGGGVVQPGRSLRLSCAASGFTFSSYGMHWVRQAPGKGLEWVTLIWFDGTTRFYADSVKGRFTVSRDNSKKTLYLQMNSLRAEDTAVYYCAREVAIQGRDYYIDVWGKGTTVTVSS

SEQ ID NO: 717 - protein, 107 aa, synthetic construct
AIQMTQSPSSLSASVGDRVTITCRASQGIRNGLGWYQQKPGKAPKLLIYAASSLQSGVPSRFSGSGSGTDFTLTISSLQPADFATYYCLQDYNYPYTFGQGTKLEIR

SEQ ID NO: 718 - protein, 122 aa, synthetic construct
QVQLVESGGGVVQPGRSLRLSCAASGFTFSSYGIHWFRQAPGKGLEWVAIIWFDGTNKFYADSVKGRFTISRDNSKNTVNLQMNSLRVEDTAIYYCAREVAIQGYYYYMDVWGNGTTVTVSS

SEQ ID NO: 719 - protein, 107 aa, synthetic construct
AIQMTQSPSSLSASVGDRVTITCRASQDIRNGLGWYQQKPGKAPMLLIYTTSNLQNGVPSRFSGSRSGTDFTLTISGLQPEDFAAYFCLQDYNYPYTFGQGTKLDVK

SEQ ID NO: 720 - protein, 123 aa, synthetic construct
QIQLVQSGPELKKPGETVKISCKASGYTFTNYGMNWVKQAPGKGLKCMGWINTNTGEPTYAEEFKGRFAFSLETSATTAFLQINNLKDEDTATYFCARLGIYYDYGYYAMDYWGQGASVTVSS

SEQ ID NO: 721 - protein, 111 aa, synthetic construct
NIVLTQSPASLAVSLGQRATISCRASESVDSYGNSFMHWYQQKPGQPPKLFIYLASNLESGVPARFSGSGSRTDFTLTIDPVEADDAATYYCQQNNEDPYTFGGGTKLEIK

SEQ ID NO: 722 - protein, 119 aa, synthetic construct
EVQLLESGGGLVQPGGSLRLSCAASEIIFSTYAMSWVRQAPGKGLEWVSGISGSGDNTYYADSVKGRFTISSDNSKNTLYLQMNSLRAEDTAVYYCVKEGYNWNYMDVWGKGTTVTVSS

SEQ ID NO: 723 - protein, 107 aa, synthetic construct
AIQMTQSPYSLSASVGDRVTITCRASQGIRNDLGWYQQKPGKAPKVLIYEASSLQSGVPSRFSGSRSGTDFTLTISSLQPEDFATYYCLQDYTYPYTFGQGTKLEIK

SEQ ID NO: 724 - protein, 119 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSS

SEQ ID NO: 725 - protein, 107 aa, synthetic construct
AIQMTQSPSSLSASVGDRVTITCRASHGIRNDLGWYQQKPGKAPKVLISETSSLQSGVPSRFSGSRSGTDFTLTISSLQPEDFATYYCLQDYTYPYTFGQGTKLEIK

SEQ ID NO: 726 - protein, 107 aa, synthetic construct
AIQMTQSPSSLSASVGDRVTITCRASHGIRNDLGWYQQKPGKAPKVLIYEASSLQSGVPSRFSGSRSGTDFTLTISSLQPEDFATYYCLQDYTYPYTFGQGTKLEIK

SEQ ID NO: 727 - protein, 118 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSS

SEQ ID NO: 728 - protein, 107 aa, synthetic construct
AIQMTQSPSSLSASVGDRVTITCRASQGIRNDLGWCQQKPGKAPKFLIYAASTLQSGVPSRFSGSGYGTDFTLTISSLQPEDFATYYCLQDYNYPRTFGQGTKVEIK

SEQ ID NO: 729 - protein, 107 aa, synthetic construct
AIQMTQSPSSLSASVGDRVTITCRASQGIRNDLGWYQQKPGKAPKFLIYAASTLQSGVPSRFSGSGYGTDFTLTISSLQPEDFATYYCLQDYNYPRTFGQGTKVEIK

SEQ ID NO: 730 - protein, 119 aa, synthetic construct
EVQLLESGGGLVQPGGSLRLSCAASGFTFSTNAMSWVRQAPGKGLEWVSGISGSNGNTYYADSVKGRFIISRDISKNTLYLEMNSLRAEDTAVYYCAKERDNWNGFDYWGQGTLVTVSS

SEQ ID NO: 731 - protein, 107 aa, synthetic construct
AIQMTQSPSSLSASVGDRVTITCRASHVIRNDLGWYQQKPGKAPKLLIYAASSLQSGVPSRFSGSGSGTDFTLTISSLQPEDFATYYCLQDYNYPYTFGQGTKLEIK

SEQ ID NO: 732 - protein, 122 aa, synthetic construct
QVQLVQSGAEVKKPGASVKVSCKVSGHTLTELSMHWVRQAPGKGLEWMGIFDPRDGQIIYAEKFQGRVTVTEDTSTDTAYMELSSLRFDDTAVYYCATGGNEDYYFYYMDVWGKGTTVTVSS

SEQ ID NO: 733 - protein, 107 aa, synthetic construct
ETTLTQSPAFMSATPGDKVNISCKASQDIDDDVNWYQQKPGEAAIFIIQEATTLVPGIPPRFSGSGYGTDFTLTINNIESEDAAYYFCLQHDHFPYTFGQGTKLEIK

SEQ ID NO: 734 - protein, 59 aa, synthetic construct
  VARIANT 59: note = K, R
MSVEHADIWVKSYSLYSRERYICNSGFKRKAGTSSLTECVLNKATNVAHWTTPSLKCIX

SEQ ID NO: 735 - protein, 65 aa, synthetic construct
  VARIANT 65: note = K, R
ITCPPPMSVEHADIWVKSYSLYSRERYICNSGFKRKAGTSSLTECVLNKATNVAHWTTPSLKCIX

SEQ ID NO: 736 - protein, 59 aa, synthetic construct
MSVEHADIWVKSYSLYSRERYICNSGFKRKAGTSSLTECVLNKATNVAHWTTPSLKCIR

SEQ ID NO: 737 - protein, 59 aa, synthetic construct
MSVEHADIWVKSYSLYSRERYICNSGFKRKAGTSSLTECVLNKATNVAHWTTPSLKCIK

SEQ ID NO: 738 - protein, 65 aa, synthetic construct
ITCPPPMSVEHADIWVKSYSLYSRERYICNSGFKRKAGTSSLTECVLNKATNVAHWTTPSLKCIR

SEQ ID NO: 739 - protein, 65 aa, synthetic construct
ITCPPPMSVEHADIWVKSYSLYSRERYICNSGFKRKAGTSSLTECVLNKATNVAHWTTPSLKCIK

SEQ ID NO: 740 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 741 - protein, 114 aa, synthetic construct
DWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 742 - protein, 114 aa, synthetic construct
NWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 743 - protein, 114 aa, synthetic construct
NWVNVIGDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 744 - protein, 114 aa, synthetic construct
NWVNVISGLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 745 - protein, 114 aa, synthetic construct
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 746 - protein, 114 aa, synthetic construct
NWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 747 - protein, 114 aa, synthetic construct
NWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 748 - protein, 114 aa, synthetic construct
NWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 749 - protein, 114 aa, synthetic construct
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 750 - protein, 114 aa, synthetic construct
NWVNVISDLKKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTS

SEQ ID NO: 751 - protein, 114 aa, synthetic construct
NWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFINTS

SEQ ID NO: 752 - protein, 114 aa, synthetic construct
NWVNVISDLKKIEDLIQSMHIDATLYTESDVHPSCKVTAMKCFLLELQVISLESGDASIHDTVENLIILAENSLSSNGNVTESGCKECEELEEKNIKEFLQSFVHIVQMFINTS

SEQ ID NO: 753 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 754 - protein, 114 aa, synthetic construct
NWVNVISDLKKIEDLIQSMHIDATLYTESDVHPSCKVTAMKCFLLELQVISLESGDASIHDTVENLIILANNSLSSLGNVTESGCKECEELEEKNIKEFLQSFVHIVQMFINTS

SEQ ID NO: 755 - protein, 114 aa, synthetic construct
NWVNVISDLKKIEDLIQSMHIDATLYTESDVHPSCKVTAMKCFLLELQVISLESGDASIHDTVENLIILANNSLSSNGGVTESGCKECEELEEKNIKEFLQSFVHIVQMFINTS

SEQ ID NO: 756 - protein, 114 aa, synthetic construct
NWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFINTS

SEQ ID NO: 757 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 758 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIKTS

SEQ ID NO: 759 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIDTS

SEQ ID NO: 760 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFISTS

SEQ ID NO: 761 - protein, 114 aa, synthetic construct
DWVDVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 762 - protein, 114 aa, synthetic construct
DWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 763 - protein, 114 aa, synthetic construct
DWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 764 - protein, 114 aa, synthetic construct
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 765 - protein, 114 aa, synthetic construct
NWVDVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 766 - protein, 114 aa, synthetic construct
NWVDVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 767 - protein, 114 aa, synthetic construct
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 768 - protein, 114 aa, synthetic construct
NWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 769 - protein, 114 aa, synthetic construct
NWVNVISGLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 770 - protein, 114 aa, synthetic construct
NWVNVISGLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 771 - protein, 114 aa, synthetic construct
NWVNVISGLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 772 - protein, 114 aa, synthetic construct
NWVNVISNLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 773 - protein, 114 aa, synthetic construct
NWVNVISNLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 774 - protein, 114 aa, synthetic construct
NWVNVISNLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 775 - protein, 114 aa, synthetic construct
NWVNVISDLKKIEDLIQSMHIDATLYTESNVHPSCKVTAMKCFLLELQVISLESGDASIHDTVEDLIILANNSLSSNGNVTESGCKECEELEEKNIKEFLQSFVHIVQMFINTS

SEQ ID NO: 776 - protein, 114 aa, synthetic construct
NWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVQMFINTS

SEQ ID NO: 777 - protein, 114 aa, synthetic construct
NWVNVISDLKKIEDLIQSMHIDATLYTESDVHPSCKVTAMKCFLLELQVISLESGDASIHNTVEDLIILANNSLSSNGNVTESGCKECEELEEKNIKEFLQSFVHIVQMFINTS

SEQ ID NO: 778 - protein, 114 aa, synthetic construct
NWVNVISDLKKIEDLIQSMHIDATLYTESDVHPSCKVTAMKCFLLELQVISLESGDASIHDTVQDLIILANNSLSSNGNVTESGCKECEELEEKNIKEFLQSFVHIVQMFINTS

SEQ ID NO: 779 - protein, 114 aa, synthetic construct
NWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 780 - protein, 114 aa, synthetic construct
NWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVEMFINTS

SEQ ID NO: 781 - protein, 114 aa, synthetic construct
NWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 782 - protein, 114 aa, synthetic construct
NWVNVISDLKKIEDLIQSMHIDATLYTESDVHPSCKVTAMKCFLLELQVISLESGDASIHNTVQDLIILANNSLSSNGNVTESGCKECEELEEKNIKEFLQSFVHIVQMFINTS

SEQ ID NO: 783 - protein, 114 aa, synthetic construct
NWVNVISDLKKIEDLIQSMHIDATLYTESDVHPSCKVTAMKCFLLELQVISLESGDASIHNTVEDLIALANNSLSSNGNVTESGCKECEELEEKNIKEFLQSFVHIVQMFINTS

SEQ ID NO: 784 - protein, 114 aa, synthetic construct
NWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFINTS

SEQ ID NO: 785 - protein, 114 aa, synthetic construct
NWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFIDTS

SEQ ID NO: 786 - protein, 114 aa, synthetic construct
NWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFIDTS

SEQ ID NO: 787 - protein, 114 aa, synthetic construct
NWVNVIGDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 788 - protein, 114 aa, synthetic construct
NWVNVIGDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 789 - protein, 114 aa, synthetic construct
NWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTS

SEQ ID NO: 790 - protein, 114 aa, synthetic construct
NWVNVISDLKKIEDLIQSMHIDATLYTESDVHPSCKVTAMKCFLLELQVISLESGDASIHDTVENLIILAANSLSSNGAVTESGCKECEELEEKNIKEFLQSFVHIVQMFIATS

SEQ ID NO: 791 - protein, 114 aa, synthetic construct
NWVNVISDLKKIEDLIQSMHIDATLYTESDVHPSCKVTAMKCFLLELQVISLESGDASIHDTVENLIILAGNSLSSNGGVTESGCKECEELEEKNIKEFLQSFVHIVQMFIGTS

SEQ ID NO: 792 - protein, 114 aa, synthetic construct
NWVNVISDLKKIEDLIQSMHIDATLYTESDVHPSCKVTAMKCFLLELQVISLESGDASIHDTVENLIILASNSLSSNGLVTESGCKECEELEEKNIKEFLQSFVHIVQMFIETS

SEQ ID NO: 793 - protein, 114 aa, synthetic construct
NWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIKTS

SEQ ID NO: 794 - protein, 114 aa, synthetic construct
NWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIKTS

SEQ ID NO: 795 - protein, 114 aa, synthetic construct
NWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIDTS

SEQ ID NO: 796 - protein, 114 aa, synthetic construct
NWVNVISDLKKIEDLIQSMHIDATLYTESDVHPSCKVTAMKCFLLELQVISLESGDASIHDTVENLIILAKNSLSSNGPVTESGCKECEELEEKNIKEFLQSFVHIVQMFIDTS

SEQ ID NO: 797 - protein, 114 aa, synthetic construct
NWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFISTS

SEQ ID NO: 798 - protein, 114 aa, synthetic construct
NWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIDTS

SEQ ID NO: 799 - protein, 114 aa, synthetic construct
DWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 800 - protein, 114 aa, synthetic construct
NWVDVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 801 - protein, 114 aa, synthetic construct
NWVNVISDLKKIEDLIQSMHIDATLYTESDVHPSCKVTAMKCFLLELQVISLESGDASIHDTVENLIILAQNSLSSLGQVTESGCKECEELEEKNIKEFLQSFVHIVQMFIQTS

SEQ ID NO: 802 - protein, 114 aa, synthetic construct
NWVNVISDLKKIEDLIQSMHIDATLYTESDVHPSCKVTAMKCFLLELQVISLESGDASIHDTVENLIILAANSLSSLGAVTESGCKECEELEEKNIKEFLQSFVHIVQMFIATS

SEQ ID NO: 803 - protein, 114 aa, synthetic construct
NWVNVISDLKKIEDLIQSMHIDATLYTESDVHPSCKVTAMKCFLLELQVISLESGDASIHDTVENLIILAGNSLSSLGGVTESGCKECEELEEKNIKEFLQSFVHIVQMFIGTS

SEQ ID NO: 804 - protein, 114 aa, synthetic construct
NWVNVISDLKKIEDLIQSMHIDATLYTESDVHPSCKVTAMKCFLLELQVISLESGDASIHDTVENLIILASNSLSSLGLVTESGCKECEELEEKNIKEFLQSFVHIVQMFIETS

SEQ ID NO: 805 - protein, 114 aa, synthetic construct
NWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIKTS

SEQ ID NO: 806 - protein, 114 aa, synthetic construct
NWVNVISDLKKIEDLIQSMHIDATLYTESDVHPSCKVTAMKCFLLELQVISLESGDASIHDTVENLIILALNSLSSLGPVTESGCKECEELEEKNIKEFLQSFVHIVQMFIKTS

SEQ ID NO: 807 - protein, 114 aa, synthetic construct
NWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFISTS

SEQ ID NO: 808 - protein, 114 aa, synthetic construct
NWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIDTS

SEQ ID NO: 809 - protein, 114 aa, synthetic construct
NWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFIDTS

SEQ ID NO: 810 - protein, 114 aa, synthetic construct
NWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIDTS

SEQ ID NO: 811 - protein, 114 aa, synthetic construct
NWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFISTS

SEQ ID NO: 812 - protein, 114 aa, synthetic construct
NWVNVIGDLKKIEDLIQSMHIDATLYTESDVHPSCKVTAMKCFLLELQVISLESGDASIHDTVEDLIILALNSLSSLGPVTESGCKECEELEEKNIKEFLQSFVHIVQMFIKTS

SEQ ID NO: 813 - protein, 114 aa, synthetic construct
NWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIKTS

SEQ ID NO: 814 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 815 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 816 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 817 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 818 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISGLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 819 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 820 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 821 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 822 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 823 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 824 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTS

SEQ ID NO: 825 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFINTS

SEQ ID NO: 826 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSNGNVTESGCKECEELEEKNIKEFLQSFVHIVQMFINTS

SEQ ID NO: 827 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 828 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSLGNVTESGCKECEELEEKNIKEFLQSFVHIVQMFINTS

SEQ ID NO: 829 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGGVTESGCKECEELEEKNIKEFLQSFVHIVQMFINTS

SEQ ID NO: 830 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFINTS

SEQ ID NO: 831 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 832 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIKTS

SEQ ID NO: 833 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIDTS

SEQ ID NO: 834 - protein, 198 aa, synthetic construct
  VARIANT 59: note = K or R
  VARIANT 62..83: note = XX is G or GG
  VARIANT 60..84: note = 1 to 5 units GGXXS; XS is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFISTS

SEQ ID NO: 835 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVDVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 836 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 837 - protein, 198 aa, synthetic construct
  VARIANT 59: note = K or R
  VARIANT 62..83: note = XX is G or GG
  VARIANT 60..83: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 838 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 839 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVDVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 840 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVDVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 841 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 842 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 843 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISGLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 844 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISGLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 845 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISGLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 846 - protein, 198 aa, synthetic construct
  VARIANT 59: note = K or R
  VARIANT 62..83: note = G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISNLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 847 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISNLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 848 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISNLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 849 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESNVHPSCKVTAMKCFLLELQVISLESGDASIHDTVEDLIILANNSLSSNGNVTESGCKECEELEEKNIKEFLQSFVHIVQMFINTS

SEQ ID NO: 850 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVQMFINTS

SEQ ID NO: 851 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHNTVEDLIILANNSLSSNGNVTESGCKECEELEEKNIKEFLQSFVHIVQMFINTS

SEQ ID NO: 852 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVQDLIILANNSLSSNGNVTESGCKECEELEEKNIKEFLQSFVHIVQMFINTS

SEQ ID NO: 853 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 854 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVEMFINTS

SEQ ID NO: 855 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 856 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHNTVQDLIILANNSLSSNGNVTESGCKECEELEEKNIKEFLQSFVHIVQMFINTS

SEQ ID NO: 857 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHNTVEDLIALANNSLSSNGNVTESGCKECEELEEKNIKEFLQSFVHIVQMFINTS

SEQ ID NO: 858 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFINTS

SEQ ID NO: 859 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFIDTS

SEQ ID NO: 860 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFIDTS

SEQ ID NO: 861 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 862 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 863 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTS

SEQ ID NO: 864 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAANSLSSNGAVTESGCKECEELEEKNIKEFLQSFVHIVQMFIATS

SEQ ID NO: 865 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAGNSLSSNGGVTESGCKECEELEEKNIKEFLQSFVHIVQMFIGTS

SEQ ID NO: 866 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILASNSLSSNGLVTESGCKECEELEEKNIKEFLQSFVHIVQMFIETS

SEQ ID NO: 867 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIKTS

SEQ ID NO: 868 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIKTS

SEQ ID NO: 869 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIDTS

SEQ ID NO: 870 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAKNSLSSNGPVTESGCKECEELEEKNIKEFLQSFVHIVQMFIDTS

SEQ ID NO: 871 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFISTS

SEQ ID NO: 872 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIDTS

SEQ ID NO: 873 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSDWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 874 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVDVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 875 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAQNSLSSLGQVTESGCKECEELEEKNIKEFLQSFVHIVQMFIQTS

SEQ ID NO: 876 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAANSLSSLGAVTESGCKECEELEEKNIKEFLQSFVHIVQMFIATS

SEQ ID NO: 877 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAGNSLSSLGGVTESGCKECEELEEKNIKEFLQSFVHIVQMFIGTS

SEQ ID NO: 878 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILASNSLSSLGLVTESGCKECEELEEKNIKEFLQSFVHIVQMFIETS

SEQ ID NO: 879 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIKTS

SEQ ID NO: 880 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILALNSLSSLGPVTESGCKECEELEEKNIKEFLQSFVHIVQMFIKTS

SEQ ID NO: 881 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFISTS

SEQ ID NO: 882 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIDTS

SEQ ID NO: 883 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFIDTS

SEQ ID NO: 884 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIDTS

SEQ ID NO: 885 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFISTS

SEQ ID NO: 886 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILALNSLSSLGPVTESGCKECEELEEKNIKEFLQSFVHIVQMFIKTS

SEQ ID NO: 887 - protein, 198 aa, synthetic construct
  VARIANT 59: note = K or R
  VARIANT 62..83: note = XX is G or GG
  VARIANT 60..84: note = 1 to 5 units GGXXS; XX is G or GG
MSVEHADIWVKSYSLYSRERYICNSGFKRKAGTSSLTECVLNKATNVAHWTTPSLKCIXGGXXSGGXXSGGXXSGGXXSGGXXSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIKTS

SEQ ID NO: 888 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 889 - protein, 227 aa, synthetic construct
KYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 890 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 891 - protein, 227 aa, synthetic construct
KYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 892 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 893 - protein, 228 aa, synthetic construct
ESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 894 - protein, 227 aa, synthetic construct
KYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 895 - protein, 228 aa, synthetic construct
ESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 896 - protein, 227 aa, synthetic construct
KYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 897 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVLHEALHSHYTQKSLSLSLG

SEQ ID NO: 898 - protein, 227 aa, synthetic construct
KYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVLHEALHSRFTQKSLSLSLGK

SEQ ID NO: 899 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVLHEALHSRFTQKSLSLSLG

SEQ ID NO: 900 - protein, 227 aa, synthetic construct
KYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVLHEALHSHYTQKSLSLSLGK

SEQ ID NO: 901 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 902 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 903 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 904 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 905 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 906 - protein, 231 aa, synthetic construct
EPKSSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 907 - protein, 227 aa, synthetic construct
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 908 - protein, 231 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 909 - protein, 227 aa, synthetic construct
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 910 - protein, 231 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 911 - protein, 227 aa, synthetic construct
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 912 - protein, 231 aa, synthetic construct
EPKSSDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 913 - protein, 227 aa, synthetic construct
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 914 - protein, 231 aa, synthetic construct
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPG

SEQ ID NO: 915 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSRFTQKSLSLSPGK

SEQ ID NO: 916 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVLHEALHSRFTQKSLSLSPG

SEQ ID NO: 917 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 918 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 919 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 920 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 921 - protein, 449 aa, synthetic construct
EVQLLESGGGLVQPGGSLRLSCAASGFSFSNYAMSWVRQAPGKGLEWVASINEGGSTFYADSVKSRFTISRDNSKNTLYLEMNSLRAEDTAVFYCSRHYGGSYDPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 922 - protein, 214 aa, synthetic construct
DIQMTQSPSSLSASVGDRVTITCRASQDINKYIAWYQQKPGKGPKLLIHYTSTLHPGVPSRFSGSGSGTDYTLTISSLQPEDFATYYCLQYDNPLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 923 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 924 - protein, 446 aa, synthetic construct
EVQLLESGGGLVQPGGSLRLSCAASGFSFSNYAMSWVRQAPGKGLEWVASINEGGSTFYADSVKSRFTISRDNSKNTLYLEMNSLRAEDTAVFYCSRHYGGSYDPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 925 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 926 - protein, 446 aa, synthetic construct
EVQLLESGGGLVQPGGSLRLSCAASGFSFSNYAMSWVRQAPGKGLEWVASINEWGSTFYADSVKSRFTISRDNSKNTLYLEMNSLRAEDTAVFYCSRHYGGSYDPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 927 - protein, 446 aa, synthetic construct
EVQLLESGGGLVQPGGSLRLSCAASGFSFSNYAMSWVRQAPGKGLEWVASINEYGSTFYADSVKSRFTISRDNSKNTLYLEMNSLRAEDTAVFYCSRHYGGSYDPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 928 - protein, 214 aa, synthetic construct
DIQMTQSPSSLSASVGDRVTITCRASQDINKYIAWYQQKPGKGPKLLIHYTSILHPGVPSRFSGSGSGTDYTLTISSLQPEDFATYYCLQYDNPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 929 - protein, 214 aa, synthetic construct
DIQMTQSPSSLSASVGDRVTITCRASQDINKYIAWYQQKPGKGPKLLIHYTSILHPGVPSRFSGSGSGTDYTLTISSLQPEDFATYYCLQYDNPLQTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 930 - protein, 449 aa, synthetic construct
EVQLLESGGGLVQPGGSLRLSCAASGFSFSNYAMSWVRQAPGKGLEWVASINEGGSTFYADSVKGRFTISRDNSKNTLYLQMNSLRAEDTAVFYCSRHYGGSYDPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 931 - protein, 446 aa, synthetic construct
EVQLLESGGGLVQPGGSLRLSCAASGFSFSNYAMSWVRQAPGKGLEWVASINEGGSTFYADSVKGRFTISRDNSKNTLYLQMNSLRAEDTAVFYCSRHYGGSYDPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 932 - protein, 449 aa, synthetic construct
EVQLLESGGGLVQPGGSLRLSCAASGFSFSNYAMSWVRQAPGKGLEWVASINEGGSTFYADSVKGRFTISRDNSKNTLYLQMNSLRAEDTAVFYCSRHYGGSYDPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 933 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 934 - protein, 446 aa, synthetic construct
EVQLLESGGGLVQPGGSLRLSCAASGFSFSNYAMSWVRQAPGKGLEWVASINEGGSTFYADSVKGRFTISRDNSKNTLYLQMNSLRAEDTAVFYCSRHYGGSYDPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 935 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 936 - protein, 448 aa, synthetic construct
EVQLVESGGGVVRPGGSLRLSCAPSGFTFDDYGMSWVRQAPGKGLEWVSGINWNGDSTGYADSVRGRFTISRDNAKNSLYLQMNSLRAEDTALYHCARDGAIGGMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 937 - protein, 214 aa, synthetic construct
AIQMTQSPSSLSASVGDRVTITCRASQDIRDDLGWYQQKPGKAPKLLIYSASTLQSGVPSRFSGSESGTDFTLTISSLQPEDFAAYYCLQDYNYPWTFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 938 - protein, 452 aa, synthetic construct
QVQLVQSGAEVRKPGASVKVSCKVSGHTLTELSMHWIRQAPGEGLEWMGYFDPRGGETIYAQKFQGRVTLTEDTSTDTAYMELTSLTSEDTAVYYCATGGDRDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 939 - protein, 214 aa, synthetic construct
ETTLTQSPAFMSATPGDKVNISCKASQDIDDDMNWFQRKPGEAAIFIIQEATTLVPGVPPRFSGSGYGTDFTLTISNIESEDAAYYFCLQHDSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 940 - protein, 452 aa, synthetic construct
QVQLLQSGAEVKRPGASVRVSCKVSGFTLTKLSMHWVRQAPGKGLEWMGFFDPRDGERIYAQKFQGRVTMTEDTSTDTAYMELSSLRSEDTAVYYCATGGDYDYYYYYMGVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 941 - protein, 214 aa, synthetic construct
ETTLTQSPAFMSATPGDKVNISCKASQDIDDDLNWYQQKPGEAAIFIVQEATTLVPGIPPRFSGSGYGTDFTLTINNIESEDAAYYFCLQHDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 942 - protein, 452 aa, synthetic construct
QVQLVQSGAEVKKPGASVKVSCKVSGHTVTELSMHWVRQAPGKGLEWMGSFHPRDDEIIYAQKFQGRVTMTEDSFTDTAYMELSSLTSEDTAVYYCATGGNPDYYFYYMDVWGKGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 943 - protein, 214 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 944 - protein, 452 aa, synthetic construct
QVQLLQSGAEVKKPGASVKVSCKVSGHTLTELSMHWVRQAPGKGLEWMGSFHPRDGETIYAQKFQGRVTLTEDTSKDTAFMELSRLRSEDTAVYYCATGGNKDYYFYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 945 - protein, 214 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 946 - protein, 452 aa, synthetic construct
QVQLVQSGAEVKKPGASVKVSCKVSGYTLTALSMHWVRQAPGKGLEWMGYFNPRDGETIYAQKFQGRVTMTEDTFTDTAYMELSSLRSEDTAIYYCATGGDSDYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 947 - protein, 214 aa, synthetic construct
ETTLTQSPAFMSATPGDKVNISCKASQDIDDDMNWYQQKPGEAAIFIIQEATTLVPGIPPRFSGSGYGTDFTLIINNIESEDAAYYFCLQHDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 948 - protein, 452 aa, synthetic construct
QVQLVQSGAEVKKPGASVKVSCKVSGHTLTELSMHWVRQAPGTGLEWMGYFNPRDDETIYTQKFQGRVTMTEDTSTDTAYMELRSLRSEDTAVYYCATGGNKDYYFYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 949 - protein, 214 aa, synthetic construct
ETTLTQSPAFMSATPGDKVTISCKASQDIDDDMNWYQQKPGEAAIFIIQEATTLVPGIPPRFSGSGYGTDFTLTINNIESEDAAYYFCLQHDNFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 950 - protein, 452 aa, synthetic construct
QVQLVQSGAEVKKPGASVKVSCKVSGHTLTELSMHWVRQAPGKGLEWMGVFDPKYGETTYAQKFQDRVTMTEDTSTDTAYMELSSLRSEDTAVYYCATGGNYEYFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 951 - protein, 214 aa, synthetic construct
ETTLTQSPAFMSATPGDKVNISCKASQDIDDDMNWYQQKPGEAAIFIIQEATTLVPGIPPRFSGSGYGTDFTLTINNIESEDAAYYFCLQHDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 952 - protein, 452 aa, synthetic construct
QVQLIQSGAEVKKPGASVKVSCKVSGHTLTELSMHWVRQAPGKGLEWMGIFDPKYGETMYAQKFQGRVTLTEDTSTDTAYMELSSLRSEDTAVYYCATGGSPDYFYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 953 - protein, 214 aa, synthetic construct
ETTLTQSPAFMSATPGDKVNISCKASQDIDDDMNWYQQKPGEAAIFVIQEATTLVPGIPRRFSGSGYGTDFTLTINNIESEDAAYYFCLQHDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 954 - protein, 452 aa, synthetic construct
QVQLLQSGAEVKKPGASVKVSCKVSGHTLTELSMHWVRQAPGKGLEWMGIFDPRNGETIYAQKFQGRVTMTEDTSTDTAYMELSSLRSEDTAVYYCATGGSPDYYYWYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 955 - protein, 214 aa, synthetic construct
ETTLTQSPAFMSATPGDKVNISCKASQDIDDDMNWYQQKPGEPAIFIIQEATTLVPGIPPRFSGSGYGTDFTLTINNIESEDAAYYFCLQHDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 956 - protein, 452 aa, synthetic construct
QVQLLQSGAEVKKPGASVKVSCKVSGHTLTELSMHWVRQAPGKGLEWMGIFDPRNGETIYAQKFQGRVTMTEDTSTDTAYMELSSLSSEDTAVYYCATGGSPDYYFWYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 957 - protein, 451 aa, synthetic construct
QVQLVQSGAEVKKPGASVKVSCKASGYTFTSYDINWVRQATGQGLEWMGWMNPNSGNTGSTQKFQGRVTMTRNTSITTAYMELSSLRSEDTAVYYCARGVLKGDYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 958 - protein, 215 aa, synthetic construct
EIVMTQSPATLSLSPGERATLSCRASQSISSSYLSWYQQKPGQAPRLLIYGSSTRATGIPARFSGSGSGTDFTLTISSLQPEDFAVYYCQQDDHLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 959 - protein, 451 aa, synthetic construct
QVQLVHSGAEVKKPGASVKVSCKASGYTFTSYDINWVRQATGQGLEWMGWVNPNSGNTGSTQKFQGRVTMTRNTSISTAYMELSSLRSEDTAVYYCARGVLKGDYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 960 - protein, 215 aa, synthetic construct
EIVMTQSPATLSLSPGERATLSCRASQSISSTYLSWYQQKPGQAPRLLIYGSSTRATGIPARFSGSGSGTDFTLTISSLQPEDFAVYYCQQDDHLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 961 - protein, 451 aa, synthetic construct
QVQLVQSGAEVKKPGASVKVSCKASGYTFTSYDINWVRQATGQGLEWMGWVNPNSGNTGSTQKFQGRVTMTRNTSISTAYMELSSLRSEDTAVYYCARGVLKGDYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 962 - protein, 215 aa, synthetic construct
EIVMTQSPATLSLSPGERATLSCRASQSISSTYLSWYQQKPGQAPRLLIYGSSTRATGIPARFRGSGSGTDFTLTISSLQPEDFAVYYCQQDDHLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 963 - protein, 455 aa, synthetic construct
QVQLVQSGAEVKKPGASVKVSCKVSGYTLTELSMHWVRQAPEKGLEWMGGFDPEDGKTIYAPKFQGRVTMTEDTSTDTAYMDLSSLRSEDTAVYYCATGHNWNDGYYFYYYMDLWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 964 - protein, 219 aa, synthetic construct
DIVMTQTPLSLSVTPGQPASISCKSSQSLLHTDGKTYLYWYLQKPGQPPHLLIYEVSNRFSGVPDRLSGSGSGTDFTLKISRVEAEDVGVYYCMQSLQLPLTFGGGTMVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 965 - protein, 455 aa, synthetic construct
QVQLVESGGDSVKPGGSLRLSCAASGFTFSDYYMNWIRQAPGKGLEWISYISSSGNTIFYVDSVEGRFTVSRDNAKNSLYLQMNSLRAEDTAVYYCAREGYNNYNYSYYYFMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 966 - protein, 214 aa, synthetic construct
EIVMTQSPATLSVSPGERATLSCRASQSVSRKLAWYQQKPGQAPRLLIYAASTRATGIPARFSGSGSGTEFTLTISSLQSEDFAVYFCQQYNNWPYTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 967 - protein, 452 aa, synthetic construct
QVQLVESGGGVVQPGRSLRLSCAASGFTVSSFGMHWVRQAPGKGLEWVAIIWYDGTNKYYADSVKGRFTISRDNSKNTLYLQLNSLRGEDTAVYYCAREIAVDGTDYYMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 968 - protein, 214 aa, synthetic construct
AIQMTQSPSSLSASVGDRVTFTCRASQGIRSGLGWYQQKPGKAPNLLIYAASTLQSGVPSRFSGSGSGTDFTLTITSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 969 - protein, 455 aa, synthetic construct
QVQLVESGGGLVTPGGSLRLSCAASGFTFSDYYMNWIRQAPGKGLEWLSYISSSGLTIFYVDSVKGRFTVSRDNAKNSLYLQMNSLRAEDTAVYYCAREGYSGFDDHYYYYTDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 970 - protein, 214 aa, synthetic construct
EIVMTQSPATLSVSPGERATLSCRASQSVSSNLAWYQQKPGQAPRLLIYGASIRATGIPARFSGSGSGTEFTLTISSLQSEDFAVYYCQHYKNWPYTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 971 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 972 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 973 - protein, 452 aa, synthetic construct
QVQLVESGGGLVTPGGSLRLSCAASGFTFSDYYMNWIRQAPGKGLEWLSYISSSGLTIFYVDSVKGRFTVSRDNAKNSLYLQMNSLRAEDTAVYYCAREGYSGFDDHYYYYTDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 974 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 975 - protein, 455 aa, synthetic construct
QVQLVESGGGLVKPGGSLRLSCAASGFTFSDYYMNWIRQAPGKGLEWVSYISSSGSTIFYTDSVKGRFTISRDNAKNSLYLQMNSLRAEDTAVYYCAREDYSNYEDYYYYYTGVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 976 - protein, 214 aa, synthetic construct
EIVMTQSPAALSVSPGERATLSCRASQSVSSKFAWYQQKPGQAPRLLIYNASTRATGIPARFSGSGSGTEFTLTISSLQSEDFAVYYCQQYNNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 977 - protein, 455 aa, synthetic construct
QVQLVESGGGLVKPGGSLRLSCSASGFTFSDYYMNWIRQAPGKGLEWVSYISSSGITIFYTDSVKGRFTISRDNAKNSLYLQMNSLRAEDTAVYYCAREDYSNYEDHYYYYTGVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 978 - protein, 214 aa, synthetic construct
EIVMTQSPAALSVSPGERATLSCRASQSVSNKFAWYQQKPGQAPRLLIYNASTRATSIPARFSGSGSGTEFTLTISSLQSEDFAVYYCQHYNNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 979 - protein, 214 aa, synthetic construct
EIVMTQSPAALSVSPGERATLSCRASQSVSNKFAWYQQKPGQAPRLLIYGASTRATSIPARFSGSGSGTEFTLTISSLQSEDFAVYYCQHYNNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 980 - protein, 452 aa, synthetic construct
QVQLVESGGGLVKPGGSLRLSCSASGFTFSDYYMNWIRQAPGKGLEWVSYISSSGITIFYTDSVKGRFTISRDNAKNSLYLQMNSLRAEDTAVYYCAREDYSNYEDHYYYYTGVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 981 - protein, 455 aa, synthetic construct
QVQLVESGGGLVKPGGSLRLSCAASGFTFSDYYMNWIRQAPGKGLEWVSYISSSGKTIFYTDSVKGRFTISRDNAKNSLFLQMNSLRAEDTAVYYCAREDYSNYEDYYYYYTGVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 982 - protein, 214 aa, synthetic construct
EIVMTQSPAALSVSPGERATLSCRASQSVSSKFAWYQQKPGQAPRLLIYNASTRATGIPARFSGSGSGTEFTLTISSLQSEDFAVYYCHQYKNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 983 - protein, 214 aa, synthetic construct
EIVMTQSPAALSVSPGERATLSCRASQSVSSKFAWYQQKPGQAPRLLIYGASTRATGIPARFSGSGSGTEFTLTISSLQSEDFAVYYCHQYKNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 984 - protein, 452 aa, synthetic construct
QVQLVESGGGLVKPGGSLRLSCAASGFTFSDYYMNWIRQAPGKGLEWVSYISSSGKTIFYTDSVKGRFTISRDNAKNSLFLQMNSLRAEDTAVYYCAREDYSNYEDYYYYYTGVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 985 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 986 - protein, 455 aa, synthetic construct
QVQLVESGGGLVKPGGSLRLSCAASGFTFSDYYMNWIRQAPGKGLEWISYISISGQTIYYGDSVKGRFTVSRDNAKNSLFLEMNSLRAEDSAVYFCAREGYSNYGVKYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 987 - protein, 214 aa, synthetic construct
EILMTQSPATLSVSPGERTTLSCRASQSISSNLAWYQQNPGQAPRLLIYNASTRATDIPARFSGGGSGTEFTLTISSLQSEDFAVYYCQQYNAWTYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 988 - protein, 456 aa, synthetic construct
QVQLVESGGGLVKPGGSLRLSCAASGFTFSDHYMNWVRQVPGKGLEWISYISISGNTIYYTDSVKGRFTVSRDNAKNSLYLQMTSLRAEDTAVYYCVREGYSSSSRGDYSYYTDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 989 - protein, 214 aa, synthetic construct
EIMMTQSPATLSVSPGERATLSCRASQSVSSNLAWYQQKPGQAPRLLIYGASTRATGIPARFSGSGSGTEFTLTISSLQSEDFAVYYCQQYKNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 990 - protein, 456 aa, synthetic construct
QVQLVESGGGLVKPGGSLRLSCAASGFTFSDYYMNWVRQVPGKGLEWISYISISGRTIYYIDSVKGRFTVSRDNAKNSLYLQMTNLRAEDTAVYYCVREGYSSSSRGDYSYYTDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 991 - protein, 214 aa, synthetic construct
EIVMTQSPATLSASPGERVNLSCRASQGVSSNLAWYQQKVGQAPRLLIYGASTRATDIPARFSGSGSGSEFTLTISSLQSEDFAVYYCQHYKNWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 992 - protein, 453 aa, synthetic construct
QVQLVESGGGLVKPGGSLRLSCAASGFTFSDYYMNWVRQVPGKGLEWISYISISGRTIYYIDSVKGRFTVSRDNAKNSLYLQMTNLRAEDTAVYYCVREGYSSSSRGDYSYYTDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 993 - protein, 452 aa, synthetic construct
QVQLVESGGGVVQPGRSLRLSCAASGFTVSSYGMHWVRQAPGKGLEWVAIIWFDGSNKYYADSLTGLFTISRDSSKNTLFLQMNSLSLEDTAVYYCAREVALEGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 994 - protein, 214 aa, synthetic construct
AIQMTQSPSSLSASVGDRVTITCRASQDIRNGLGWYQQKPGKAPKLLIYTTSSLQTGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 995 - protein, 452 aa, synthetic construct
QVQLVESGGGVVQPGRSLSLSCAASGFTVSSYGMHWVRQAPGKGLEWVAILWFDGSNKFYADSVKGRFTISRDNSKNTLYLQMNSLRVEDTAVYYCARELALEGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 996 - protein, 214 aa, synthetic construct
AIQMTQSPSSLSASVGDRVTITCRASQDIRNGLGWYQQNPGKAPKLLIYTTSSLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 997 - protein, 452 aa, synthetic construct
QVQLVESGGGVVQPGRSLRLSCAASGFTVSSYGMHWVRQAPGKGLEWVAILWFDGSNQFYADSVKGRFTISRDNSKNTLYLQMNSLRVEDTAVYYCAREIALEGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 998 - protein, 214 aa, synthetic construct
AIQMTQSPSSLSASVGDRVTITCRSSQDIRNGLGWYQQKPGKAPKLLIYTTSSLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 999 - protein, 452 aa, synthetic construct
QVQLVESGGGVVQPGRALRLSCAASGFTFSSYGMHWVRQAPGKGLEWVAIIWYDGSNKYYADSVKGRFTISRDNSKNTLYLQMNSLRAEDTAVYYCAREVALEGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1000 - protein, 214 aa, synthetic construct
DIQMTQSPSSLSASVGDRVTITCRASQDIRNGLGWYQQKPGKAPKLLIYTTSNLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1001 - protein, 452 aa, synthetic construct
QVQLVESGGGVVQPGRSLRLSCAASGFTFSSYGMHWVRQAPGKGLEWVAIIWYDGNNKYYADSVKGRFTISRDNSKNTLYLQMNSLRAEDTAVYYCTREVALEGYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1002 - protein, 214 aa, synthetic construct
AIQMTQSPSSLSASVGDRVTITCRASQGIRNGLGWYQLKPGKAPKLLIYAASSLQSGVPSSFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1003 - protein, 449 aa, synthetic construct
EVQLLESGGGLVQPGGSLRLSCAASGFTFSSYAMSWVRQAPGKGLEWVSGISGSVGSTYYADSVKGRFTISRDKSKNTLYLQMNSLRAEDTAVYYCAKEGYDWSGMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1004 - protein, 214 aa, synthetic construct
AIQMTQSPSSLSAFVGDRVTITCRASQGIRNDLGWYQQKPGKAPKLLIYAASSLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1005 - protein, 452 aa, synthetic construct
QVLLVESGGGVVQPGRSLRLSCAASGFTFSSYGMHWVRQAPGKGLEWVTLIWFDGTTRFYADSVKGRFTVSRDNSKKTLYLQMNSLRAEDTAVYYCAREVAIQGRDYYIDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1006 - protein, 214 aa, synthetic construct
AIQMTQSPSSLSASVGDRVTITCRASQGIRNGLGWYQQKPGKAPKLLIYAASSLQSGVPSRFSGSGSGTDFTLTISSLQPADFATYYCLQDYNYPYTFGQGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1007 - protein, 452 aa, synthetic construct
QVQLVESGGGVVQPGRSLRLSCAASGFTFSSYGIHWFRQAPGKGLEWVAIIWFDGTNKFYADSVKGRFTISRDNSKNTVNLQMNSLRVEDTAIYYCAREVAIQGYYYYMDVWGNGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1008 - protein, 214 aa, synthetic construct
AIQMTQSPSSLSASVGDRVTITCRASQDIRNGLGWYQQKPGKAPMLLIYTTSNLQNGVPSRFSGSRSGTDFTLTISGLQPEDFAAYFCLQDYNYPYTFGQGTKLDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1009 - protein, 449 aa, synthetic construct
EVQLLESGGGLVQPGGSLRLSCAASEIIFSTYAMSWVRQAPGKGLEWVSGISGSGDNTYYADSVKGRFTISSDNSKNTLYLQMNSLRAEDTAVYYCVKEGYNWNYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1010 - protein, 214 aa, synthetic construct
AIQMTQSPYSLSASVGDRVTITCRASQGIRNDLGWYQQKPGKAPKVLIYEASSLQSGVPSRFSGSRSGTDFTLTISSLQPEDFATYYCLQDYTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1011 - protein, 449 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1012 - protein, 214 aa, synthetic construct
AIQMTQSPSSLSASVGDRVTITCRASHGIRNDLGWYQQKPGKAPKVLISETSSLQSGVPSRFSGSRSGTDFTLTISSLQPEDFATYYCLQDYTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1013 - protein, 446 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 1014 - protein, 214 aa, synthetic construct
AIQMTQSPSSLSASVGDRVTITCRASHGIRNDLGWYQQKPGKAPKVLIYEASSLQSGVPSRFSGSRSGTDFTLTISSLQPEDFATYYCLQDYTYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1015 - protein, 448 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1016 - protein, 214 aa, synthetic construct
AIQMTQSPSSLSASVGDRVTITCRASQGIRNDLGWCQQKPGKAPKFLIYAASTLQSGVPSRFSGSGYGTDFTLTISSLQPEDFATYYCLQDYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1017 - protein, 214 aa, synthetic construct
AIQMTQSPSSLSASVGDRVTITCRASQGIRNDLGWYQQKPGKAPKFLIYAASTLQSGVPSRFSGSGYGTDFTLTISSLQPEDFATYYCLQDYNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1018 - protein, 445 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 1019 - protein, 448 aa, synthetic construct
EVQLLESGGGLVQPGGSLRLSCSASGFTFSDYVMSWVRQAPGKGLDWVSSISGLTGNTYHADSVKGRFTISRDNSKNTLYLQMSSLRDEDTAVYYCAKDNEDYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1020 - protein, 214 aa, synthetic construct
AIQMTQSPSSLSASVGDRVTITCRASQGIRNDLGWYQQKPGKAPKFLISAASSLQSGVPSRFSGSGYGTDFTLTISSLQPEDFATYYCLQDYTYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1021 - protein, 445 aa, synthetic construct
EVQLLESGGGLVQPGGSLRLSCSASGFTFSDYVMSWVRQAPGKGLDWVSSISGLTGNTYHADSVKGRFTISRDNSKNTLYLQMSSLRDEDTAVYYCAKDNEDYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSLGK

SEQ ID NO: 1022 - protein, 449 aa, synthetic construct
EVQLLESGGGLVQPGGSLRLSCAASGFTFSTNAMSWVRQAPGKGLEWVSGISGSNGNTYYADSVKGRFIISRDISKNTLYLEMNSLRAEDTAVYYCAKERDNWNGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1023 - protein, 214 aa, synthetic construct
AIQMTQSPSSLSASVGDRVTITCRASHVIRNDLGWYQQKPGKAPKLLIYAASSLQSGVPSRFSGSGSGTDFTLTISSLQPEDFATYYCLQDYNYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1024 - protein, 452 aa, synthetic construct
QVQLVQSGAEVKKPGASVKVSCKVSGHTLTELSMHWVRQAPGKGLEWMGIFDPRDGQIIYAEKFQGRVTVTEDTSTDTAYMELSSLRFDDTAVYYCATGGNEDYYFYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1025 - protein, 214 aa, synthetic construct
ETTLTQSPAFMSATPGDKVNISCKASQDIDDDVNWYQQKPGEAAIFIIQEATTLVPGIPPRFSGSGYGTDFTLTINNIESEDAAYYFCLQHDHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1026 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSLGPVTESGCKECEELEEKNIKEFLQSFVHIVQMFIKTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1027 - protein, 449 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1028 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSTSGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1029 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSLGPVTESGCKECEELEEKNIKEFLQSFVHIVQMFIKTSTSGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1030 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1031 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1032 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSLGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1033 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGPVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1034 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFIKTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1035 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1036 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFISTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1037 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1038 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGNVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1039 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGPVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1040 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAQNSLSSNGQVTESGCKECEELEEKNIKEFLQSFVHIVQMFIQTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1041 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAANSLSSNGAVTESGCKECEELEEKNIKEFLQSFVHIVQMFIATSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1042 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAGNSLSSNGGVTESGCKECEELEEKNIKEFLQSFVHIVQMFIGTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1043 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILASNSLSSNGLVTESGCKECEELEEKNIKEFLQSFVHIVQMFIETSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1044 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSNGPVTESGCKECEELEEKNIKEFLQSFVHIVQMFIKTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1045 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFIKTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1046 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1047 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAKNSLSSNGPVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1048 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSNGPVTESGCKECEELEEKNIKEFLQSFVHIVQMFISTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1049 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSNGPVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1050 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAQNSLSSLGQVTESGCKECEELEEKNIKEFLQSFVHIVQMFIQTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1051 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAANSLSSLGAVTESGCKECEELEEKNIKEFLQSFVHIVQMFIATSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1052 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAGNSLSSLGGVTESGCKECEELEEKNIKEFLQSFVHIVQMFIGTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1053 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILASNSLSSLGLVTESGCKECEELEEKNIKEFLQSFVHIVQMFIETSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1054 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIKTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1055 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFISTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1056 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIDTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1057 - protein, 436 aa, synthetic construct
  VARIANT 97: note = G, S
  VARIANT 155: note = D, N
  VARIANT 161: note = A, E, G, K, L, N, Q, S
  VARIANT 167: note = N, L
  VARIANT 169: note = A, G, L, N, P, Q, S
  VARIANT 202: note = A, D, E, G, K, Q, S
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIXDLKKIEDLIQSMHIDATLYTESDVHPSCKVTAMKCFLLELQVISLESGDASIHDTVEXLIILAXNSLSSXGXVTESGCKECEELEEKNIKEFLQSFVHIVQMFIXTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1058 - protein, 452 aa, synthetic construct
EVQLVESGGGLVQPGGSLRLSCVASGLTFSTFAMSWVRQAPGKGLEWVSGISGSGENTYYADSVKGRFTISSDNAKNTLYLQMNSLRAEDTAVYYCAKEGYNWNYMDVWGKGTTVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPASIQKTISKDKGQPREPQVYTLPPSREELTKNQVSLSCAVKGFYPSDIVVEWESSGQPENTYKTTPPVLDSDGSYFLVSKLTVDKSRWQQGNVFSCSVMHEALHNRYTQKSLSVSPGK

SEQ ID NO: 1059 - protein, 214 aa, synthetic construct
DIQMTQSPSSLSASVGDRVTITCRASHGIRNDLGWYQQKPGKAPKVLIYEASSLQSGVPSRFSGSRSGTDFTLTISSLQPEDFATYYCLQDYTYPYTFGQGTKLEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 1060 - protein, 436 aa, synthetic construct
ITCPPPVSVEHADIRVKSYSLYSRERYICNSGFKRKAGTSSLTECVLNKATNIAHWTTPSLKCIRGGGGSGGGGSGGGGSGGGGSGGGGSNWVNVISDLKKIEDLIQSMHIDATLYTESDVHPSCKVTAMKCFLLELQVISHESGDTDAHDTVEDLIILANNILSSNGNITESGCKECEELEEKNIKEFLQSFVHIVQMFINTSTSGGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPASIQKTISKDKGQPREPQVYTLPPSREELTKNQVSLWCLVKGFYPSDIVVEWESSGQPENTYKTTPPVLDSDGSYFLYSKLTVDKSRWQQGNVFSCSVMHEALHNHYTQKSLSVSPGK

SEQ ID NO: 1061 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1062 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1063 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1064 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1065 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1066 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1067 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 1068 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVEDLIALANNSLSSNGNVTESGCKECEELEEKNIKEFLQSFVHIVQMFINTS

SEQ ID NO: 1069 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1070 - protein, 669 aa, synthetic construct
EVELLESGGGLVQPGGSLRLSCAASGLTFSTFAMSWVRQAPGKGLEWVSGISGSGENTYYADSVKGRFTITSDNSKNTLYLQMNSLRAEDTAVYYCAKEGYNWNYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVEMFINTS

SEQ ID NO: 1071 - protein, 443 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRYTQKSLSLSL

SEQ ID NO: 1072 - protein, 668 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1073 - protein, 668 aa, synthetic construct
EVQLLESGGGLVQPGGSLRLSCAASGFTFSTYAMSWVRQAPGKGLEWVSTISDNIGNTYYADSVKGRFTISRDNSKNTLYLQMNSLRAEDTAVYYCTKDNEDYYMDVWGKGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1074 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1075 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1076 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1077 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGGVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1078 - protein, 16 aa, synthetic construct
SINEGGSTFYADSVKG

SEQ ID NO: 1079 - protein, 16 aa, synthetic construct
SINDGGSTFYADSVKG

SEQ ID NO: 1080 - protein, 16 aa, synthetic construct
SINAGGSTFYADSVKG

SEQ ID NO: 1081 - protein, 16 aa, synthetic construct
SINDAGSTFYADSVKG

SEQ ID NO: 1082 - protein, 17 aa, synthetic construct
SFHPRDGETIYAQKFQG

SEQ ID NO: 1083 - protein, 12 aa, synthetic construct
VKEGYNWNYMDV

SEQ ID NO: 1084 - protein, 8 aa, synthetic construct
SQSISSSY

SEQ ID NO: 1085 - protein, 25 aa, synthetic construct
GGGGSGGGGSGGGGSGGGGSGGGGS

SEQ ID NO: 1086 - protein, 452 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1087 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1088 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSTSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1089 - protein, 453 aa, synthetic construct
QIQLVQSGPELKKPGETVKISCKASGYTFTNYGMNWVKQAPGKGLKCMGWINTNTGEPTYAEEFKGRFAFSLETSATTAFLQINNLKDEDTATYFCARLGIYYDYGYYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 1090 - protein, 218 aa, synthetic construct
NIVLTQSPASLAVSLGQRATISCRASESVDSYGNSFMHWYQQKPGQPPKLFIYLASNLESGVPARFSGSGSRTDFTLTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1091 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1092 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVEDLIALANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1093 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVGVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1094 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1095 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1096 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1097 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVGVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1098 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVADLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1099 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHATVEDLIILANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1100 - protein, 436 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQDLIALANNSLSSNGNVTESGCKECEELEEKNIKEFLQSFVHIVQMFINTSGGGGGKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1101 - protein, 452 aa, synthetic construct
EVQLVESGGGLAKPGGSLRLSCAASGFSFSNYAMSWVRQAPGKGLEWVASINDGGSTFYADSVKGRFTISRENAKNTLYLQMNSLRAEDTAVYYCSRHYGGSYDPMDYWGQGVLVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPASIQKTISKDKGQPREPQVYTLPPSREELTKNQVSLSCAVKGFYPSDIVVEWESSGQPENTYKTTPPVLDSDGSYFLVSKLTVDKSRWQQGNVFSCSVMHEALHNRYTQKSLSVSPGK

SEQ ID NO: 1102 - protein, 214 aa, synthetic construct
DIQMTQSPSSLSASVGDRVTITCRASQDINKYIAWYQQKPGKGPKLLIHYTSTLHPGVPSRFSGSGSGTDYTLTISSLQPEDFATYYCLQYDNPLYTFGGGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 1103 - protein, 436 aa, synthetic construct
ITCPPPVSVEHADIRVKSYSLYSRERYICNSGFKRKAGTSSLTECVLNKATNIAHWTTPSLKCIRGGGGSGGGGSGGGGSGGGGSGGGGSNWVNVISDLKKIEDLIQSMHIDATLYTESDVHPSCKVTAMKCFLLELQVISHESGDTDIHDTVEDLIILANNILSSNGNITESGCKECEELEEKNIKEFLQSFVHIVQMFINTSTSGGGSKPPTCPPCPAPEAAGGPSVFLFPPKPKDTLMISRTPEVTCVVVDVSQEDPDVKFNWYVNGAEVHHAQTKPRETQYNSTYRVVSVLTVTHQDWLNGKEYTCKVSNKALPASIQKTISKDKGQPREPQVYTLPPSREELTKNQVSLWCLVKGFYPSDIVVEWESSGQPENTYKTTPPVLDSDGSYFLYSKLTVDKSRWQQGNVFSCSVMHEALHNHYTQKSLSVSPGK

SEQ ID NO: 1104 - DNA, 1347 bp, synthetic construct
  source 1..1347: mol_type = other DNA
gaagtgcagctgctggaatctggcggaggactggttcaacctggcggctctctgagactgtcttgtgccgccagcggcttcagcttcagcaactacgccatgagctgggtccgacaggcccctggaaaaggccttgaatgggtcgccagcatcaacgaaggcggcagcaccttttacgccgacagcgtgaagtcccggttcaccatcagccgggacaacagcaagaacaccctgtacctggaaatgaacagcctgagagccgaggacaccgccgtgttctactgcagcagacactacggcggcagctacgaccccatggattattggggccagggcaccaccgtgacagtgtctagcgcctctacaaagggccccagcgttttcccactggctcctagcagcaagagcacaagcggaggaacagccgctctgggctgtctggtcaaggactactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaagctgcaggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgccagcatcgagaaaaccatcagcaaggccaagggccagcctagggaaccccaggtttacacactgcctccaagccgggaagagatgaccaagaaccaggtgtccctgagctgtgccgtgaagggcttctacccttccgatatcgccgtggaatgggagagcaatggccagccagagaacaactacaagacaacccctcctgtgctggacagcgacggctcattcttcctggtgtccaagctgacagtggacaagtccagatggcagcagggcaacgtgttcagctgcagcgtgatgcacgaggccctgcacaaccggtacacccagaagtctctgtctctgagccccggcaaa

SEQ ID NO: 1105 - DNA, 642 bp, synthetic construct
  source 1..642: mol_type = other DNA
gacatccagatgacccagagccccagctccctgagcgccagcgtgggcgacagggtgaccatcacctgcagagccagccaggacatcaacaagtacatcgcctggtatcagcagaagcccggcaagggccccaaacttttgatccactacactagcaccctgcaccccggagtgcccagcaggttcagcggcagtggcagcggcaccgactacaccctcaccattagcagcctgcaacccgaggacttcgccacctactactgcctgcagtacgacaaccccctgtacaccttcggccagggcaccaagttggagatcaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgc

SEQ ID NO: 1106 - DNA, 1308 bp, synthetic construct
  source 1..1308: mol_type = other DNA
atcacatgccctccacctatgagcgtggaacacgccgacatctgggtcaagagctacagcctgtacagcagagagcggtacatctgcaacagcggcttcaagagaaaggccggcaccagcagcctgaccgagtgtgtgctgaacaaggccaccaatgtggcccactggaccacacctagcctgaagtgtattagaggcggcggaggatctggcggaggtggaagcggaggcggaggaagcggtggcggcggtagtggcggtggtggatctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgataccgtggaagatctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcaccagcggcggcagcgacaagacacatacctgtcctccatgtcctgctccagaagctgctggcggcccttccgtgtttctgttccctccaaagcctaaggacaccctgatgatctccagaactcccgaagtgacctgcgtggtggtggacgtgtcccacgaagatcctgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgccagcatcgagaaaaccatcagcaaggccaagggccagcctagggaaccccaggtttacacactgcctccaagccgggaagagatgaccaagaaccaggtgtccctgtggtgcctggtcaagggcttctacccttccgatatcgccgtggaatgggagagcaatggccagcctgagaacaactacaagacaacccctcctgtgctggacagcgacggctcattcttcctgtactccaagctgacagtggacaagagcagatggcagcagggcaacgtgttcagctgcagcgtgatgcatgaggccctgcacaaccactacacccagaagtccctgagcctgtctcctggcaaa

SEQ ID NO: 1107 - DNA, 1338 bp, synthetic construct
  source 1..1338: mol_type = other DNA
gaagtgcagctgctggaatctggcggaggactggttcaacctggcggctctctgagactgtcttgtgccgccagcggcttcagcttcagcaactacgccatgagctgggtccgacaggcccctggaaaaggccttgaatgggtcgccagcatcaacgaaggcggcagcaccttttacgccgacagcgtgaagtcccggttcaccatcagccgggacaacagcaagaacaccctgtacctggaaatgaacagcctgagagccgaggacaccgccgtgttctactgcagcagacactacggcggcagctacgaccccatggattattggggccagggcaccaccgtgacagtgtctagcgcctctacaaagggccccagcgttttcccactggctccctgtagcagaagcaccagcgaatctacagccgctctgggctgcctggtcaaggactactttcctgagcctgtgaccgtgtcctggaacagcggagcactgacatctggcgtgcacacctttccagccgtgctgcaaagcagcggcctgtactctctgagcagcgtggtcacagtgcctagctctagcctgggcaccaagacctacacctgtaatgtggaccacaagcctagcaacaccaaggtggacaagcgcgtggaatctaagtacggccctccttgtcctccatgtcctgctccagaagctgcaggcggcccttccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccggtacacccagaagtctctgtccctgtctctgggcaaa

SEQ ID NO: 1108 - DNA, 1308 bp, synthetic construct
  source 1..1308: mol_type = other DNA
atcacatgccctccacctatgagcgtggaacacgccgacatctgggtcaagagctacagcctgtacagcagagagcggtacatctgcaacagcggcttcaagagaaaggccggcaccagcagcctgaccgagtgtgtgctgaacaaggccaccaatgtggcccactggaccacacctagcctgaagtgtattagaggcggcggaggatctggcggaggtggaagcggaggcggaggaagcggtggcggcggtagtggcggtggtggatctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgataccgtggaagatctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcggcggcggaggtggaaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgtggtgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtctcttggcaag

SEQ ID NO: 1109 - DNA, 1338 bp, synthetic construct
  source 1..1338: mol_type = other DNA
gaggtgcagctgctggaatctggcggaggattggttcagcctggcggctctctgagactgtcttgtgccgcctctggcttctccttctccaactacgccatgtcctgggtccgacaggctcctggcaaaggactggaatgggtcgcctccatcaacgaaggcggctctaccttctacgccgactccgtgaagtcccggttcaccatctccagagacaactccaagaacaccctgtacctggaaatgaactccctgagagccgaggacaccgccgtgttctactgctctagacactacggcggcagctacgaccccatggattattggggccagggcaccacagtgaccgtgtcctctgcttctaccaagggacccagcgtgttccctctggctccttgctccagatccacctccgagtctacagctgctctgggctgcctggtcaaggactactttcctgagcctgtgacagtgtcctggaactccggcgctctgacatctggcgtgcacacctttccagctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtccagctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatcagcaaggccaagggccagcctagggaaccccaggtttacaccctgcctccaagccaagaggaaatgaccaagaaccaggtgtccctgtcctgcgccgtgaagggcttctacccttctgatatcgccgtggaatgggagagcaacggccagcctgagaacaactacaagacaacccctcctgtgctggactccgacggctctttctttctggtgtcccgcctgaccgtggacaagtctagatggcaagagggcaacgtgttctcctgcagcgtgatgcacgaggccctgcacaacagatacacccagaagtccctgtctctgtccctgggcaaa

SEQ ID NO: 1110 - DNA, 642 bp, synthetic construct
  source 1..642: mol_type = other DNA
gacatccagatgacccagtctccatcctctctgtccgcctctgtgggcgacagagtgaccatcacctgtagagccagccaggacatcaacaagtatatcgcctggtatcagcagaagcccggcaagggacctaagctgctgatccactacacctccacactgcaccctggcgtgccctctagattttccggctctggctctggcaccgactataccctgacaatctccagcctgcagcctgaggacttcgccacctactactgcctgcagtacgacaaccctctgtacacctttggccagggcaccaagctggaaatcaagcggacagtggccgctccttccgtgttcatcttcccaccttccgacgagcagctgaagtccggcacagcttctgtcgtgtgcctgctgaacaacttctaccctcgggaagctaaggtgcagtggaaggtggacaacgccctgcagtccggcaactcccaagagtctgtgaccgagcaggactccaaggacagcacctacagcctgtccagcactctgaccctgtccaaggccgactacgagaagcacaaggtgtacgcctgcgaagtgacccatcagggcctgtctagccctgtgaccaagtctttcaaccggggcgagtgt

SEQ ID NO: 1111 - DNA, 1308 bp, synthetic construct
  source 1..1308: mol_type = other DNA
atcacctgtcctccacctatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgtattagaggcggcggaggatctggcggaggtggaagtggcggtggtggttcaggcggtggcggaagcggaggcggtggatctaattgggtcaacgtgatctccgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaagatctgatcatcctggccaacaactccctgtccagcaacggcaacgtgaccgagtctggctgcaaagagtgcgaggaactggaagagaagaacatcaaagagttcctccagtccttcgtgcacatcgtgcagatgttcatcaacacctccggcggaggcggagggaagtatggacctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgtggtgcctggtcaagggcttctacccttccgatatcgccgtggaatgggagagcaatggccagcctgagaacaactacaagacaacccctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggcaaa

SEQ ID NO: 1112 - DNA, 1347 bp, synthetic construct
  source 1..1347: mol_type = other DNA
gaggtcgagctcctggagagcggcgggggcctcgtccagcccggcggcagcctcagactgagctgcgcggctagtggactgaccttttccacctttgccatgtcatgggtgcggcaggcgccaggcaaaggactggagtgggtatctggaattagcggctccggggagaacacatattacgctgactctgtaaaaggaaggtttacgatcactagtgataactccaagaatactctttatctgcagatgaatagcctgagggcagaggatacagccgtgtactattgcgccaaggaaggctacaattggaactacatggacgtgtgggggaagggcaccacagtcaccgtatcttccgccagcaccaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgccagcatcgagaaaaccatcagcaaggccaagggccagcctagggaaccccaggtttacacactgcctccaagccgggaagagatgaccaagaaccaggtgtccctgagctgtgccgtgaagggcttctacccttccgatatcgccgtggaatgggagagcaatggccagcctgagaacaactacaagacaacccctcctgtgctggacagcgacggctcattcttcctggtgtccaagctgacagtggacaagtccagatggcagcagggcaacgtgttcagctgcagcgtgatgcacgaggccctgcacaaccggtacacccagaagtccctgtctctgagccccggcaaa

SEQ ID NO: 1113 - DNA, 642 bp, synthetic construct
  source 1..642: mol_type = other DNA
gcgatccagatgacccaatccccctcctctctgagcgcaagtgttggcgaccgagtgacgatcacttgtagggcctcacatggcatacggaacgacctgggctggtatcagcagaagccaggaaaggcacccaaagtgctgatttatgaagcttcatcacttcagtctggggtgcccagtcgattttcaggttcaagatccgggaccgacttcactctcaccatcagctccttgcagcccgaggacttcgcgacctactattgtcttcaggactacacatatccatatactttcggtcaggggacgaaacttgagataaaaagaacagtggccgctccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 1114 - DNA, 1308 bp, synthetic construct
  source 1..1308: mol_type = other DNA
atcacatgccctccacctatgagcgtggaacacgccgacatctgggtcaagagctacagcctgtacagcagagagcggtacatctgcaacagcggcttcaagagaaaggccggcaccagcagcctgaccgagtgtgtgctgaacaaggccaccaatgtggcccactggaccacacctagcctgaagtgtattagaggcggcggaggatctggcggaggtggaagcggaggcggaggaagcggtggcggcggtagtggcggtggtggatctaattgggtcaacgtgatcggcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgataccgtggaagatctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacctctacctctggtggttctgacaagacccacacctgtcctccatgtcctgctccagaagctgctggcggcccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgccagcatcgagaaaaccatcagcaaggccaagggccagcctagggaaccccaggtttacacactgcctccaagccgggaagagatgaccaagaaccaggtgtccctgtggtgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgcagcgtgatgcacgaggccctgcacaaccactacacccagaagtccctgagcctgtctcctggcaaa

SEQ ID NO: 1115 - DNA, 1347 bp, synthetic construct
  source 1..1347: mol_type = other DNA
gaggtggaactgctggaatctggcggaggattggttcagcctggcggctctctgagactgtcttgtgctgcttctggcctgaccttctccaccttcgccatgtcctgggtccgacaggctcctggcaaaggactggaatgggtgtccggcatctctggctctggcgagaatacctactacgccgactccgtgaagggcagattcaccatcacctccgacaactccaagaacaccctgtacctgcagatgaactccctgagagccgaggacaccgccgtgtactactgtgccaaagagggctacaactggaactacatggacgtgtggggcaagggcaccaccgtgacagtttcttccgcctccaccaagggacccagcgttttccctctggctccatcctccaagtctacctctggcggaacagctgctctgggctgcctggtcaaggactactttcctgagcctgtgaccgtgtcctggaactccggtgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtccagctccagaagctgctggcggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcctccatcgaaaagaccatctccaaggccaagggccagcctagggaaccccaggtttacaccctgccacctagccgggaagagatgaccaagaaccaggtgtccctgtcctgcgctgtgaagggcttctacccttccgatatcgccgtggaatgggagagcaatggccagcctgagaacaactacaagacaacccctcctgtgctggactccgacggctcattcttcctggtgtccaagctgacagtggacaagtccagatggcagcagggcaacgtgttctcctgctccgtgatgcacgaggccctgcacaacagatacacccagaagtccctgtctctgtcccctggcaaa

SEQ ID NO: 1116 - DNA, 642 bp, synthetic construct
  source 1..642: mol_type = other DNA
gccatccagatgacccagtctccatcctctctgtccgcctctgtgggcgacagagtgaccatcacctgtagagccagccacggcatcagaaacgacctcggctggtatcagcagaagcctggcaaggctcccaaggtgctgatctacgaggcttccagtctgcagtctggcgtgccctctaggttctccggctctaggtctggcaccgactttaccctgacaatctccagcctgcagcctgaggacttcgccacctactactgcctgcaagactacacctatccttacaccttcggccagggcaccaagctggaaatcaagagaaccgtggccgctccttccgtgttcatcttcccaccttccgacgagcagctgaagtccggcacagcttctgtcgtgtgcctgctgaacaacttctaccctcgggaagccaaggtgcagtggaaggtggacaatgctctgcagtccggcaactcccaagagtccgtgaccgagcaggactccaaggactctacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 1117 - DNA, 1308 bp, synthetic construct
  source 1..1308: mol_type = other DNA
atcacctgtcctccacctatgtccgtggaacacgccgacatctgggtcaagtcctacagcctgtactccagagagcggtacatctgcaactccggcttcaagagaaaggccggcacctctagcctgaccgagtgcgtgctgaacaaggccaccaatgtggcccactggaccacacctagcctgaagtgtattagaggcggcggaggatctggcggaggtggaagcggaggcggaggaagcggtggcggcggtagtggcggtggtggatctaattgggtcaacgtgatcggcgacctgaagaagatcgaggacctgatccagtccatgcacatcgacgctaccctgtacaccgagtccgacgtgcacccttcctgtaaagtgaccgccatgaagtgctttctgctggaactgcaagtgatctccctggaatccggcgacgcctctatccacgacaccgtggaagatctgatcatcctggccaacaactccctgtccagcaacggcaacgtgaccgagtctggctgcaaagagtgcgaggaactggaagagaagaacatcaaagagttcctccagtccttcgtgcacatcgtgcagatgttcatcaacacctccaccagcggcggctccgacaagacacatacctgtccaccttgtcctgctccagaggctgctggtggcccttccgtgtttctgttccctccaaagcctaaggacaccctgatgatctctcggacccctgaagtgacctgcgtggtggtcgatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcctccatcgaaaagaccatctccaaggccaagggccagcctagggaaccccaggtttacaccctgccacctagccgggaagagatgaccaagaaccaggtgtccctgtggtgcctggtcaagggcttctacccttccgatatcgccgtggaatgggagagcaatggccagcctgagaacaactacaagacaacccctcctgtgctggactccgacggctcattcttcctgtacagcaagctgacagtggacaagtccagatggcagcagggcaacgtgttctcctgctccgtgatgcacgaggccctgcacaatcactacacccagaagtccctgtctctgtcccctggcaaa

SEQ ID NO: 1118 - DNA, 1338 bp, synthetic construct
  source 1..1338: mol_type = other DNA
gaggtcgagctcctggagagcggcgggggcctcgtccagcccggcggcagcctcagactgagctgcgcggctagtggactgaccttttccacctttgccatgtcatgggtgcggcaggcgccaggcaaaggactggagtgggtatctggaattagcggctccggggagaacacatattacgctgactctgtaaaaggaaggtttacgatcactagtgataactccaagaatactctttatctgcagatgaatagcctgagggcagaggatacagccgtgtactattgcgccaaggaaggctacaattggaactacatggacgtgtgggggaagggcaccacagtcaccgtatcttccgcctctacaaagggccccagcgttttcccactggctccctgtagcagaagcaccagcgaatctacagccgctctgggctgcctggtcaaggactactttcctgagcctgtgaccgtgtcctggaacagcggagcactgacatctggcgtgcacacctttccagccgtgctgcaaagcagcggcctgtactctctgagcagcgtggtcacagtgcctagctctagcctgggcaccaagacctacacctgtaatgtggaccacaagcctagcaacaccaaggtggacaagcgcgtggaatctaagtacggccctccttgtcctccatgtcctgctccagaagctgcaggcggcccttccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgacagtgctgcaccaggattggctgaacggcaaagagtacaagtgcaaggtgtccaacaagggcctgccaagcagcatcgagaaaaccatcagcaaggccaagggccagcctagggaaccccaggtttacacactgcctccaagccaagaggaaatgaccaagaaccaggtgtccctgagctgcgccgtgaagggcttttacccttccgatatcgccgtggaatgggagagcaatggccagccagagaacaactacaagacaacccctcctgtgctggacagcgacggctcattcttcctggtgtccagactgaccgtggacaagagcagatggcaagagggcaacgtgttcagctgcagcgtgatgcacgaggccctgcacaaccggtacacccagaagtctctgtccctgtctctgggcaaa

SEQ ID NO: 1119 - DNA, 1308 bp, synthetic construct
  source 1..1308: mol_type = other DNA
atcacatgccctccacctatgagcgtggaacacgccgacatctgggtcaagagctacagcctgtacagcagagagcggtacatctgcaacagcggcttcaagagaaaggccggcaccagcagcctgaccgagtgtgtgctgaacaaggccaccaatgtggcccactggaccacacctagcctgaagtgtattagaggcggcggaggatctggcggaggtggaagcggaggcggaggaagcggtggcggcggtagtggcggtggtggatctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgataccgtggaagatctgatcgccctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacctctggtggtggcggagggaagtacggacctccttgtcctccatgtcctgctccagaagctgctggcggcccttccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagctccatcgagaaaaccatcagcaaggccaagggccagccaagagaaccccaggtgtacacactgcctccaagccaagaggaaatgaccaagaaccaggtgtccctgtggtgcctggtcaagggcttctacccttccgatatcgccgtggaatgggagagcaatggccagcctgagaacaactacaagacaacccctcctgtgctggacagcgacggctcattcttcctgtactccagactgaccgtggacaagagcagatggcaagagggcaacgtgttcagctgcagcgtgatgcatgaggccctgcacaaccactacacccagaagtccctgagcctgtctctgggcaaa

SEQ ID NO: 1120 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 1121 - protein, 12 aa, synthetic construct
ESKYGPPCPPCP

SEQ ID NO: 1122 - protein, 15 aa, synthetic construct
EPKSCDKTHTCPPCP

SEQ ID NO: 1123 - protein, 20 aa, synthetic construct
EPKSCDKTHTCPPCPAPELL

SEQ ID NO: 1124 - protein, 115 aa, synthetic construct
DNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1125 - protein, 114 aa, synthetic construct
NWVNVISDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1126 - protein, 178 aa, synthetic construct
  VARIANT 59: note = K or R
  VARIANT 62: note = G or GG
  VARIANT 60..63: note = 1 to 5 units GGXS; X is G or GG
MSVEHADIWVKSYSLYSRERYICNSGFKRKAGTSSLTECVLNKATNVAHWTTPSLKCIXGGXSDNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1127 - protein, 177 aa, synthetic construct
  VARIANT 59: note = K or R
  VARIANT 62: note = G or GG
  VARIANT 60..63: note = 1 to 5 units GGXS; X is G or GG
MSVEHADIWVKSYSLYSRERYICNSGFKRKAGTSSLTECVLNKATNVAHWTTPSLKCIXGGXSNWVNVISDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1128 - protein, 4 aa, synthetic construct
GGGS

SEQ ID NO: 1129 - protein, 5 aa, synthetic construct
GGGGS

SEQ ID NO: 1130 - protein, 9 aa, synthetic construct
SLYNTVATL

SEQ ID NO: 1131 - protein, 6 aa, synthetic construct
DYNYPW

SEQ ID NO: 1132 - protein, 16 aa, synthetic construct
  VARIANT 15: note = G, S
INEGGSTFYADSVKXR

SEQ ID NO: 1133 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1134 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1135 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1136 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1137 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1138 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1139 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 1140 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 1141 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1142 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1143 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTS

SEQ ID NO: 1144 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFINTS

SEQ ID NO: 1145 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSNGNVTESGCKECEELEEKNIKEFLQSFVHIVQMFINTS

SEQ ID NO: 1146 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1147 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSLGNVTESGCKECEELEEKNIKEFLQSFVHIVQMFINTS

SEQ ID NO: 1148 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGGVTESGCKECEELEEKNIKEFLQSFVHIVQMFINTS

SEQ ID NO: 1149 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFINTS

SEQ ID NO: 1150 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 1151 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIKTS

SEQ ID NO: 1152 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIDTS

SEQ ID NO: 1153 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFISTS

SEQ ID NO: 1154 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVDVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 1155 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 1156 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1157 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1158 - protein, 199 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 1159 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 1160 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1161 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1162 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1163 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 1164 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1165 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISGLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1166 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISNLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 1167 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISNLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 1168 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISNLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 1169 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVEDLIILANNSLSSNGNVTESGCKECEELEEKNIKEFLQSFVHIVQMFINTS

SEQ ID NO: 1170 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVQMFINTS

SEQ ID NO: 1171 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVEDLIILANNSLSSNGNVTESGCKECEELEEKNIKEFLQSFVHIVQMFINTS

SEQ ID NO: 1172 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQDLIILANNSLSSNGNVTESGCKECEELEEKNIKEFLQSFVHIVQMFINTS

SEQ ID NO: 1173 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1174 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1175 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVEMFINTS

SEQ ID NO: 1176 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 1177 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVQDLIILANNSLSSNGNVTESGCKECEELEEKNIKEFLQSFVHIVQMFINTS

SEQ ID NO: 1178 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHNTVEDLIALANNSLSSNGNVTESGCKECEELEEKNIKEFLQSFVHIVQMFINTS

SEQ ID NO: 1179 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFINTS

SEQ ID NO: 1180 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSNGNVTESGCKECEELEEKNIKEFLQSFVHIVQMFIDTS

SEQ ID NO: 1181 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGPVTESGCKECEELEEKNIKEFLQSFVHIVQMFIDTS

SEQ ID NO: 1182 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEALIALANNSLSSNGNVTESGCKECEELEEKNIKEFLQSFVHIVQMFINTS

SEQ ID NO: 1183 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIALANNSLSSNGNVTESGCKECEELEEKNIKEFLQSFVHIVQMFINTS

SEQ ID NO: 1184 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTS

SEQ ID NO: 1185 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAANSLSSNGAVTESGCKECEELEEKNIKEFLQSFVHIVQMFIATS

SEQ ID NO: 1186 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAGNSLSSNGGVTESGCKECEELEEKNIKEFLQSFVHIVQMFIGTS

SEQ ID NO: 1187 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILASNSLSSNGLVTESGCKECEELEEKNIKEFLQSFVHIVQMFIETS

SEQ ID NO: 1188 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIKTS

SEQ ID NO: 1189 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIKTS

SEQ ID NO: 1190 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSNGPVTESGCKECEELEEKNIKEFLQSFVHIVQMFIDTS

SEQ ID NO: 1191 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAKNSLSSNGPVTESGCKECEELEEKNIKEFLQSFVHIVQMFIDTS

SEQ ID NO: 1192 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFISTS

SEQ ID NO: 1193 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSNGPVTESGCKECEELEEKNIKEFLQSFVHIVQMFIDTS

SEQ ID NO: 1194 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSDWVNVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 1195 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHNTVQNLIILANNSLSSNGNVTESGCKECEELEEKNIKEFLQSFVHIVEMFINTS

SEQ ID NO: 1196 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAQNSLSSLGQVTESGCKECEELEEKNIKEFLQSFVHIVQMFIQTS

SEQ ID NO: 1197 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAANSLSSLGAVTESGCKECEELEEKNIKEFLQSFVHIVQMFIATS

SEQ ID NO: 1198 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAGNSLSSLGGVTESGCKECEELEEKNIKEFLQSFVHIVQMFIGTS

SEQ ID NO: 1199 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILASNSLSSLGLVTESGCKECEELEEKNIKEFLQSFVHIVQMFIETS

SEQ ID NO: 1200 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIKTS

SEQ ID NO: 1201 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILALNSLSSLGPVTESGCKECEELEEKNIKEFLQSFVHIVQMFIKTS

SEQ ID NO: 1202 - protein, 198 aa, synthetic construct; identical to SEQ ID NO: 1201

SEQ ID NO: 1203 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILAENSLSSLGPVTESGCKECEELEEKNIKEFLQSFVHIVQMFIDTS

SEQ ID NO: 1204 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSNGPVTESGCKECEELEEKNIKEFLQSFVHIVQMFIDTS

SEQ ID NO: 1205 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIDTS

SEQ ID NO: 1206 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFISTS

SEQ ID NO: 1207 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILALNSLSSLGPVTESGCKECEELEEKNIKEFLQSFVHIVQMFIKTS

SEQ ID NO: 1208 - protein, 198 aa, synthetic construct
MSVEHADIWVKSYSLYSRERYICNSGFKRKAGTSSLTECVLNKATNVAHWTTPSLKCIRGGGGSGGGGSGGGGSGGGGSGGGGSNWVNVIGDLKKIEDLIQSMHIDATLYTESDVHPSCKVTAMKCFLLELQVISLESGDASIHDTVEDLIILAENSLSSLGPVTESGCKECEELEEKNIKEFLQSFVHIVQMFIKTS

SEQ ID NO: 1209 - protein, 378 aa, synthetic construct
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWGGGGSHHHHHHHH

SEQ ID NO: 1210 - protein, 378 aa, synthetic construct
KKVVLGKKGDTVELTCNASQKKNTQFHWKNSNQIKILGIQGSFLTKGPSKLSDRADSRKSLWDQGCFSMIIKNLKIEDSDTYICEVENKKEEVELLVFGLTANSDTHLLEGQSLTLTLESPPGSSPSVKCRSPGGKNIQGGRTLSVPQLERQDSGTWTCTVSQDQKTVEFKIDIVVLAFQKASSTVYKKEGEQVEFSFPLAFTLEKLTGSGELWWQAERASSSKSWITFDLKNKEVSVKRVTQDPKLQMGKKLPLHLTLPQALPQYAGSGNLTLALEAKTGKLHQEVNLVVMRATQFQENLTCEVWGPTSPKLTLSLKLENKGTTVSKQAKAVWVLNPEAGMWQCLLSDSGQVLLESNIKVVPTWGGGGSHHHHHHHH

SEQ ID NO: 1211 - protein, 445 aa, synthetic construct
EQKLISEEDLAAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 1212 - protein, 443 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1213 - protein, 445 aa, synthetic construct
EQKLISEEDLAAKKVVLGKKGDTVELTCNASQKKNTQFHWKNSNQIKILGIQGLFLTKGPSKLSDRADSRKSLWDQGCFSMIIKNLKIEDSDTYICEVENKKEEVELLVFGLTANSDTHLLEGQSLTLTLESPPGSSPSVKCRSPGGKNIQGGRTISVPQLERQDSGTWTCTVSQDQKTVEFKIDIVVLAFQKASSTVYKKEGEQVEFSFPLAFTLEKLTGSGELWWQAERASSSKSWITFDLKNKEVSVKRVTQDPKLQMGKKLPLHLTLPQALPQYAGSGNLTLALEAKTGKLHQEVNLVVMRATQFQENLTCEVWGPTSPKLTLSLKLENKGATVSKQAKAVWVLNPEAGMWQCLLSDSGQVLLESNIKVVPTWPTPVQPMALIVLGGVAGLLLFTGLGIFFCVRCRHRRRQAERMSQIKRLLSEKKTCQCPHRFQKTCSPI

SEQ ID NO: 1214 - protein, 441 aa, synthetic construct
EQKLISEEDLAAKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1215 - protein, 442 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTANSDTHLLQGQSLTLTLESPPGSSPSVQCRSPRGKNIQGGKTLSVSQLELQDSGTWTCTVLQNQKKVEFKIDIVVLAFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1216 - protein, 447 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1217 - protein, 443 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMRATQLQKNLTCEVWGPTSPKLMLSLKLENKEAKVSKREKAVWVLNPEAGMWQCLLSDGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1218 - protein, 447 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 1219 - protein, 447 aa, synthetic construct
EQKLISEEDLAAKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 1220 - protein, 365 aa, synthetic construct
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKWVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTW

SEQ ID NO: 1221 - protein, 452 aa, synthetic construct
EVKLVESGGGLVKPGGSLKLSCAASGFSFSNYAMSWVRQTPEKRLEWVASINDGGSTFYPDSVKGRFTISRDNAGNILYLQMSSLRSEDTAMFYCSRHYGGSYDPMDYWGQGTSVTVSSASTKGPSVFPLAPSSRSTSESTAALGCLVKDYFPEPVTVSWNSGSLTSGVHTFPAVLQSSGLYSLSSVVTVPSSSLGTQTYVCNVNHKPSNTKVDKRVEIKTCGGGSKPPTCPPCPAPEAAGGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREQLTKNQVKLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 1222 - protein, 214 aa, synthetic construct
DIQMTQSPSSLSASLGGKVTITCKASQDINKYIAWYQHKPGKGPRLLIHYTSTLHPGIPSRFSGSGSGRDYSFSISNLEPEDIATYYCLQYDNPLYTFGGGTKLEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 1223 - protein, 436 aa, synthetic construct
ITCPPPVSVEHADIRVKSYSLYSRERYICNSGFKRKAGTSSLTECVLNKATNIAHWTTPSLKCIRGGGGSGGGGSGGGGSGGGGSGGGGSNWVDVISDLKKIEDLIQSMHIDATLYTESDVHPSCKVTAMKCFLLELQVISHESGDTDIHDTVEDLIILANNILSSNGNITESGCKECEELEEKNIKEFLQSFVHIVQMFINTSTSGGGSKPPTCPPCPAPEAAGGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCDVSGFYPSDIVVEWESSGQPENTYKTTPPVLDSDGSYFLYSKLTVDKSRWQQGNVFSCSVMHEALHNHYTQKSLSVSPGK

SEQ ID NO: 1224 - protein, 353 aa, synthetic construct
NWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTSGGGGSEIKTSGGGSKPPTCPPCPAPPVAGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCDVSGFYPSDIVVEWESSGQPENTYKTTPPVLDSDGSYFLYSKLTVDKSRWQQGNVFSCSVMHEALHNHYTQKSLSVSPGK

SEQ ID NO: 1225 - protein, 304 aa, synthetic construct
ITCPPPVSVEHADIRVKSYSLYSRERYICNSGFKRKAGTSSLTECVLNKATNIAHWTTPSLKCIRGGGGSEIKTSGGGSKPPTCPPCPAPPVAGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREQLTKNQVKLTCLVKGFYPSDIVVEWESSGQPENTYKTTPPVLDSDGSYFLYSKLTVDKSRWQQGNVFSCSVMHEALHNHYTQKSLSVSPGK

SEQ ID NO: 1226 - protein, 353 aa, synthetic construct
NWVNVISDLKKIEDLIQSMHIDATLYTESNVHPSCKVTAMKCFLLELQVISHESGDTDIHDTVQDLIILANNILSSNGNITESGCKECEELEEKNIKEFLQSFVHIVQMFINTSGGGGSEIKTSGGGSKPPTCPPCPAPPVAGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREELTKNQVSLTCDVSGFYPSDIVVEWESSGQPENTYKTTPPVLDSDGSYFLYSKLTVDKSRWQQGNVFSCSVLHEALHSHYTQKSLSVSPGK

SEQ ID NO: 1227 - protein, 304 aa, synthetic construct
ITCPPPVSVEHADIRVKSYSLYSRERYICNSGFKRKAGTSSLTECVLNKATNIAHWTTPSLKCIRGGGGSEIKTSGGGSKPPTCPPCPAPPVAGPSVFLFPPKPKDTLMISRTPEVTCVVVDVKQEDPDVKFNWYVNGAEVHHAQTKPRETQYNSTYRVVSVLTVTHQDWLNGKEYTCKVSNKALPAPIQKTISKDKGQPREPQVYTLPPSREQLTKNQVKLTCLVKGFYPSDIVVEWESSGQPENTYKTTPPVLDSDGSYFLYSKLTVDKSRWQQGNVFSCSVLHEALHSHYTQKSLSVSPGK

SEQ ID NO: 1228 - DNA, 1347 bp, synthetic construct; identical to SEQ ID NO: 1104
  source 1..1347: mol_type = other DNA

SEQ ID NO: 1229 - DNA, 642 bp, synthetic construct; identical to SEQ ID NO: 1105
  source 1..642: mol_type = other DNA

SEQ ID NO: 1230 - DNA, 1308 bp, synthetic construct; identical to SEQ ID NO: 1106
  source 1..1308: mol_type = other DNA